(19) United States
(12) Patent Application Publication
(10) Pub. No.: US 2015/0010529 A1
(43) Pub. Date: Jan. 8, 2015

(54) THERMALLY STABLE PH20 HYALURONIDASE VARIANTS AND USES THEREOF

(71) Applicant: Ge Wei, San Diego, CA (US)

(72) Inventor: Ge Wei, San Diego, CA (US)

(21) Appl. No.: 14/323,932

(22) Filed: Jul. 3, 2014

Related U.S. Application Data
(60) Provisional application No. 61/957,567, filed on Jul. 3, 2013.

(51) Int. Cl. C12N 9/26; A61K 38/47; C12Q 1/34; A61K 45/06

(52) U.S. Cl. CPC C12N 9/2474; A61K 45/06; A61K 38/47; C12Q 1/34; C12Y 302/01035
USPC 424/94.62; 536/23.2; 435/201; 435/188; 435/320.1; 435/358; 435/18

(57) ABSTRACT

Modified PH20 hyaluronidase polypeptides that exhibit stability and activity under thermal stress conditions are provided. Also provided are compositions and formulations and uses thereof.

### RELATED APPLICATIONS

Benefit of priority is claimed to U.S. Provisional Application Ser. No. 61/957,567, to Ge Wei, entitled “Thermally Stable PH20 Hyaluronidase Variants And Uses Thereof,” filed Jul. 3, 2013.

This application is related to International PCT Application Serial No. (Attorney Docket No. 33320.03115.WO02/3115PC), filed the same day herewith, entitled “Thermally Stable PH20 Hyaluronidase Variants and Uses Thereof,” which claims priority to U.S. Provisional Application Ser. No. 61/957,567. This application also is related to Taiwanese Patent Application Serial No. 103122815 (Attorney Docket No. 33320.03115.TW02/3115TW), filed Jul. 2, 2014, entitled “Thermally Stable PH20 Hyaluronidase Variants and Uses Thereof,” which claims priority to U.S. Provisional Application Ser. No. 61/957,567.

The subject matter of each of the above-noted applications are incorporated by reference in its entirety.

### INCORPORATION BY REFERENCE OF SEQUENCE LISTING PROVIDED ELECTRONICALLY

An electronic version of the Sequence Listing is filed herewith, the contents of which are incorporated by reference in their entirety. The electronic file was created on Jul. 3, 2014 is 1.75 megabytes in size, and titled 3115seq001.txt.

### FIELD OF THE INVENTION

Modified PH20 hyaluronidase polypeptides that exhibit stability and activity under thermal stress conditions are provided. Also provided are compositions and formulations and uses thereof

### BACKGROUND

Hyaluronan (hyaluronic acid; HA) is a polypeptide that occurs in the extracellular matrix of many cells, especially in soft connective tissues. HA also occurs predominantly in skin, cartilage and in synovial fluid in mammals. Hyaluronan is the main constituent of the vitreous of the eye. HA has a role in various physiological processes, such as in water and plasma protein homeostasis (Laurent T C et al. (1992) FASEB J 6: 2397-2404). Certain diseases are associated with expression and/or production and/or accumulation of hyaluronan.

Hyaluronan-degrading enzymes, such as hyaluronidases, are enzymes that degrade hyaluronan. By catalyzing HA degradation, hyaluronan-degrading enzymes (e.g., hyaluronidases) can be used to treat diseases or disorders associated with accumulation of HA or other glycosaminoglycans. HA is a major component of the interstitial barrier, hyaluronan-degrading enzymes (e.g., hyaluronidase) increase tissue permeability and therefore can be used to increase the dispersion and delivery of therapeutic agents. Various hyaluronidases have been used therapeutically (e.g., Hydase™, Vitrase™ and Wydase™ hyaluronidases), typically as dispersing and spreading agents in combination with other therapeutic agents. Improved hyaluronan-degrading enzymes, such as hyaluronidases, and compositions containing such enzymes that can be used for treatment are needed.

### SUMMARY

Provided herein are modified PH20 polypeptide designated uber-thermophiles that exhibit thermal stability. The modified PH20 polypeptides provided herein contain an amino acid replacement in an unmodified PH20 polypeptide, whereby the polypeptide retains at least 50% of its hyaluronidase activity after incubation at 52° C. for 10 minutes compared to its hyaluronidase activity after incubation at 4° C. for 10 minutes. For example, the modified PH20 polypeptide contains an amino acid replacement(s) in an unmodified PH20 polypeptide that consists of the sequence of amino acids set forth in SEQ ID NO: 7 or is a C-terminal truncated fragment thereof that is a soluble PH20 polypeptide or a sequence of amino acids that has at least 85% sequence identity to SEQ ID NO:7 or a C-terminal truncated fragment thereof that is soluble. Included among the modified PH20 polypeptides provided herein are those that retain at least 60%, 65%, 70%, 75%, 80%, 85%, 90%, 95% or more of its hyaluronidase activity after incubation at 52° C. for 10 minutes compared to its hyaluronidase activity after incubation at 4° C. for 10 minutes.

For example, any of the modified PH20 polypeptides provided herein contain at least one amino acid replacement at an amino acid position corresponding to a position selected from among 10, 11, 13, 15, 26, 27, 28, 29, 30, 31, 32, 33, 34, 36, 37, 38, 39, 41, 46, 47, 48, 49, 50, 58, 60, 67, 69, 72, 73, 83, 84, 86, 87, 90, 92, 93, 94, 97, 98, 99, 102, 105, 114, 118, 120, 131, 132, 135, 138, 139, 141, 142, 143, 144, 146, 147, 148, 150, 151, 152, 154, 155, 156, 158, 159, 160, 161, 162, 163, 165, 170, 174, 195, 196, 197, 198, 202, 204, 205, 206, 208, 213, 215, 219, 220, 222, 234, 235, 237, 240, 247, 251, 255, 259, 260, 261, 263, 265, 271, 276, 277, 278, 282, 284, 285, 290, 292, 305, 306, 309, 310, 311, 315, 317, 318, 320, 321, 328, 342, 343, 349, 359, 368, 369, 371, 373, 374, 375, 376, 377, 379, 380, 388, 389, 393, 399, 401, 403, 406, 407, 410, 412, 413, 415, 417, 419, 421, 428, 431, 433, 434, 435, 438, 439, 440, 441, 442, 443, 445, 446 and 447, with reference to amino acid positions of the sequence set forth in SEQ ID NO:3, wherein corresponding amino acid positions are identified by alignment of the PH20 polypeptide with the polypeptide set forth in SEQ ID NO:3.

In particular examples, any of the modified PH20 polypeptides provided herein contain an amino acid replacement that is:

at a position corresponding to position 10, replacement with G or N;

at a position corresponding to position 11, replacement with G;

at a position corresponding to position 13, replacement with H;

at a position corresponding to position 15, replacement with A or V;

at a position corresponding to position 26, replacement with P, R, S, V, W or Y;

at a position corresponding to position 27, replacement with E or H;

at a position corresponding to position 28, replacement with L;

at a position corresponding to position 29, replacement with E, H, L, S or W;

at a position corresponding to position 30, replacement with A, P or R;

at a position corresponding to position 31, replacement with C, G or L;

at a position corresponding to position 32, replacement with Q, S, V or W;

at a position corresponding to position 33, replacement with G, M, R or W;

at a position corresponding to position 34, replacement with E, H or W;

at a position corresponding to position 36, replacement with G;

at a position corresponding to position 37, replacement with I or K;

at a position corresponding to position 38, replacement with Y;

at a position corresponding to position 39, replacement with Q, R or T;

at a position corresponding to position 41, replacement with D, T or W;

at a position corresponding to position 46, replacement with H;

at a position corresponding to position 47, replacement with G or R;

at a position corresponding to position 48, replacement with G or Y;

at a position corresponding to position 49, replacement with I;

at a position corresponding to position 50, replacement with C or D;

at a position corresponding to position 58, replacement with K or R;

at a position corresponding to position 60, replacement with K;

at a position corresponding to position 67, replacement with F;

at a position corresponding to position 69, replacement with A or Y;

at a position corresponding to position 72, replacement with D;

at a position corresponding to position 73, replacement with T;

at a position corresponding to position 83, replacement with G, Q or V;

at a position corresponding to position 84, replacement with D;

at a position corresponding to position 86, replacement with D, E, N or R;

at a position corresponding to position 87, replacement with M, P or V;

at a position corresponding to position 90, replacement with E, T or W;

at a position corresponding to position 92, replacement with V;

at a position corresponding to position 93, replacement with E or S;

at a position corresponding to position 94, replacement with N;

at a position corresponding to position 97, replacement with E or F;

at a position corresponding to position 98, replacement with M;

at a position corresponding to position 99, replacement with S;

at a position corresponding to position 102, replacement with H or N;

at a position corresponding to position 105, replacement with I, R or W;

at a position corresponding to position 114, replacement with G;

at a position corresponding to position 118, replacement with M;

at a position corresponding to position 120, replacement with S;

at a position corresponding to position 131, replacement with C or L;

at a position corresponding to position 132, replacement with A or C;

at a position corresponding to position 135, replacement with Q;

at a position corresponding to position 138, replacement with W;

at a position corresponding to position 139, replacement with R or V;

at a position corresponding to position 141, replacement with M, Q, W or Y;

at a position corresponding to position 142, replacement with Q;

at a position corresponding to position 143, replacement with K;

at a position corresponding to position 144, replacement with G;

at a position corresponding to position 146, replacement with V;

at a position corresponding to position 147, replacement with G, I or M;

at a position corresponding to position 148, replacement with C, H or K;

at a position corresponding to position 150, replacement with L;

at a position corresponding to position 151, replacement with Q;

at a position corresponding to position 152, replacement with A, I, M or T;

at a position corresponding to position 154, replacement with R;

at a position corresponding to position 155, replacement with A, D, F, H, L, R, S or V;

at a position corresponding to position 156, replacement with A, C or Q;

at a position corresponding to position 158, replacement with H;

at a position corresponding to position 159, replacement with A, H, N, Q or S;

at a position corresponding to position 160, replacement with Y;

at a position corresponding to position 161, replacement with A or D;

at a position corresponding to position 162, replacement with L;

at a position corresponding to position 163, replacement with K, R or S;

at a position corresponding to position 165, replacement with F;

at a position corresponding to position 170, replacement with R;

at a position corresponding to position 174, replacement with W;

at a position corresponding to position 195, replacement with H, L or N;

at a position corresponding to position 196, replacement with T;

at a position corresponding to position 197, replacement with F;

at a position corresponding to position 198, replacement with L;

at a position corresponding to position 202, replacement with M;

at a position corresponding to position 204, replacement with P;

at a position corresponding to position 205, replacement with A, E, K, L, P, S or T;

at a position corresponding to position 206, replacement with I;

at a position corresponding to position 208, replacement with L, Q or R;

at a position corresponding to position 213, replacement with E or N;

at a position corresponding to position 215, replacement with A, D, E, H, T, V or W;

at a position corresponding to position 219, replacement with A, R, S or T;

at a position corresponding to position 220, replacement with V;

at a position corresponding to position 222, replacement with N;

at a position corresponding to position 234, replacement with M;

at a position corresponding to position 235, replacement with T;

at a position corresponding to position 237, replacement with Q;

at a position corresponding to position 240, replacement with Q;

at a position corresponding to position 247, replacement with I;

at a position corresponding to position 251, replacement with L or M;

at a position corresponding to position 255, replacement with R;

at a position corresponding to position 259, replacement with K or P;

at a position corresponding to position 260, replacement with G or M;

at a position corresponding to position 261, replacement with A or F;

at a position corresponding to position 263, replacement with T;

at a position corresponding to position 265, replacement with I;

at a position corresponding to position 271, replacement with V;

at a position corresponding to position 276, replacement with E;

at a position corresponding to position 277, replacement with A, C, E or H;

at a position corresponding to position 278, replacement with G, H, K or N;

at a position corresponding to position 282, replacement with G or Q;

at a position corresponding to position 284, replacement with A, Q or S;

at a position corresponding to position 285, replacement with M or Y;

at a position corresponding to position 290, replacement with M;

at a position corresponding to position 292, replacement with V;

at a position corresponding to position 305, replacement with D or N;

at a position corresponding to position 306, replacement with D;

at a position corresponding to position 309, replacement with E, H or L;

at a position corresponding to position 310, replacement with Q or R;

at a position corresponding to position 311, replacement with G or K;

at a position corresponding to position 315, replacement with T;

at a position corresponding to position 317, replacement with N;

at a position corresponding to position 318, replacement with K, M, N or Q;

at a position corresponding to position 320, replacement with R;

at a position corresponding to position 321, replacement with A, H or R;

at a position corresponding to position 328, replacement with L or R;

at a position corresponding to position 342, replacement with A;

at a position corresponding to position 343, replacement with V;

at a position corresponding to position 349, replacement with A or E;

at a position corresponding to position 359, replacement with E;

at a position corresponding to position 368, replacement with H or K;

at a position corresponding to position 369, replacement with H;

at a position corresponding to position 371, replacement with E, F, M or T;

at a position corresponding to position 373, replacement with S;

at a position corresponding to position 374, replacement with A or V;

at a position corresponding to position 375, replacement with T;

at a position corresponding to position 376, replacement with Y;

at a position corresponding to position 377, replacement with T;

at a position corresponding to position 379, replacement with H, S or T;

at a position corresponding to position 380, replacement with I, L, P, T or V;

at a position corresponding to position 388, replacement with H;

at a position corresponding to position 389, replacement with K;

at a position corresponding to position 393, replacement with L;

at a position corresponding to position 399, replacement with R or W;

at a position corresponding to position 401, replacement with G;

at a position corresponding to position 403, replacement with F;

at a position corresponding to position 406, replacement with N;

at a position corresponding to position 407, replacement with F, H, M, P or Q;

at a position corresponding to position 410, replacement with S;

at a position corresponding to position 412, replacement with G, P or S;

at a position corresponding to position 413, replacement with Q or T;

at a position corresponding to position 415, replacement with W;

at a position corresponding to position 417, replacement with L;

at a position corresponding to position 419, replacement with L;

at a position corresponding to position 421, replacement with I or M;

at a position corresponding to position 428, replacement with P;

at a position corresponding to position 431, replacement with A or G;

at a position corresponding to position 433, replacement with L or T;

at a position corresponding to position 434, replacement with I or M;

at a position corresponding to position 435, replacement with H;

at a position corresponding to position 438, replacement with A;

at a position corresponding to position 439, replacement with C or T;

at a position corresponding to position 440, replacement with M;

at a position corresponding to position 441, replacement with T or V;

at a position corresponding to position 442, replacement with P;

at a position corresponding to position 443, replacement with M;

at a position corresponding to position 445, replacement with Y;

at a position corresponding to position 446, replacement with C, D, E or G; or

at a position corresponding to position 447, replacement with D, E or G, each with reference to amino acid positions of the sequence set forth in SEQ ID NO:3.

For example, any of the modified PH20 polypeptides provided herein contain at least one amino acid replacement that is replacement with: glycine (G) at a position corresponding to position 11; A at a position corresponding to position 15; V at a position corresponding to position 15; R at a position corresponding to position 26; S at a position corresponding to position 26; E at a position corresponding to position 27; H at a position corresponding to position 27; H at a position corresponding to position 29; S at a position corresponding to position 29; A at a position corresponding to position 30; P at a position corresponding to position 30; G at a position corresponding to position 31; L at a position corresponding to position 31; Q at a position corresponding to position 32; W at a position corresponding to position 32; G at a position corresponding to position 33; M at a position corresponding to position 33; R at a position corresponding to position 33; W at a position corresponding to position 33; E at a position corresponding to position 34; H at a position corresponding to position 34; W at a position corresponding to position 34; K at a position corresponding to position 37; Y at a position corresponding to position 38; Q at a position corresponding to position 39; R at a position corresponding to position 39; T at a position corresponding to position 39; D at a position corresponding to position 41; T at a position corresponding to position 41; W at a position corresponding to position 41; G at a position corresponding to position 48; C at a position corresponding to position 50; D at a position corresponding to position 50; K at a position corresponding to position 58; R at a position corresponding to position 58; K at a position corresponding to position 60; F at a position corresponding to position 67; A at a position corresponding to position 69; Y at a position corresponding to position 69; Q at a position corresponding to position 83; D at a position corresponding to position 84; D at a position corresponding to position 86; E at a position corresponding to position 86; R at a position corresponding to position 86; P at a position corresponding to position 87; W at a position corresponding to position 90; V at a position corresponding to position 92; E at a position corresponding to position 93; S at a position corresponding to position 93; N at a position corresponding to position 94; F at a position corresponding to position 97; M at a position corresponding to position 98; S at a position corresponding to position 99; H at a position corresponding to position 102; G at a position corresponding to position 114; M at a position corresponding to position 118; S at a position corresponding to position 120; C at a position corresponding to position 131; L at a position corresponding to position 131; A at a position corresponding to position 132; W at a position corresponding to position 138; R at a position corresponding to position 139; V at a position corresponding to position 139; M at a position corresponding to position 141; Y at a position corresponding to position 141; K at a position corresponding to position 143; G at a position corresponding to position 144; V at a position corresponding to position 146; I at a position corresponding to position 147; M at a position corresponding to position 147; C at a position corresponding to position 148; H at a position corresponding to position 148; K at a position corresponding to position 148; L at a position corresponding to position 150; Q at a position corresponding to position 151; I at a position corresponding to position 152; M at a position corresponding to position 152; T at a position corresponding to position 152; R at a position corresponding to position 154; A at a position corresponding to position 155; D at a position corresponding to position 155; F at a position corresponding to position 155; H at a position corresponding to position 155; L at a position corresponding to position 155; R at a position corresponding to position 155; S at a position corresponding to position 155; H at a position corresponding to position 158; A at a position corresponding to position 159; H at a position corresponding to position 159; N at a position corresponding to position 159; Q at a position corresponding to position 159; S at a position corresponding to position 159; Y at a position corresponding to position 160; A at a position corresponding to position 161; L at a position corresponding to position 162; K at a position corresponding to position 163; R at a position corresponding to position 163; S at a position corresponding to position 163; F at a position corresponding to position 165; W at a position corresponding to position 174; H at a position corresponding to position 195; L at a position corresponding to position 195; T at a position corresponding to position 196; F at a position corresponding to position 197; L at a position corresponding to position 198; P at a position corresponding to position 204; A at a position corresponding to position 205; E at a position corresponding to position 205; K at a position corresponding to position 205; L at a position corresponding to position 205; T at a position corresponding to position 205; I at a position corresponding to position 206; Q at a position corresponding to position 208; R at a position corresponding to position 208; E at a position corresponding to position 213; N at a position corresponding to position 213; E at a position corresponding to position 215; H at a position corresponding to position 215; T at a position corresponding to position 215; N at a position corresponding to position 222; T at a position corresponding to position 235; Q at a position corresponding to position 237; Q at a position corresponding to position 240; I at a position corresponding to position 247; L at a position corresponding to position 251; M at a position corresponding to position 251; K at a position corresponding to position 259; P at a position corresponding to position 259; M at a position corresponding to position 260; A at a position corresponding to position 261; F at a position corresponding to position 261; T at a position corresponding to position 263; V at a position corresponding to position 271; E at a position corresponding to position 276; A at a position corresponding to position 277; C at a position corresponding to position 277; N at a position corresponding to position 278; Q at a position corresponding to position 282; A at a position corresponding to position 284; Q at a position corresponding to position 284; S at a position corresponding to position 284; M at a position corresponding to position 285; V at a position corresponding to position 292; N at a position corresponding to position 305; D at a position corresponding to position 306; R at a position corresponding to position 310; G at a position corresponding to position 311; T at a position corresponding to position 315; N at a position corresponding to position 317; A at a position corresponding to position 321; R at a position corresponding to position 321; L at a position corresponding to position 328; R at a position corresponding to position 328; A at a position corresponding to position 342; H at a position corresponding to position 368; K at a position corresponding to position 368; H at a position corresponding to position 369; F at a position corresponding to position 371; S at a position corresponding to position 373; T at a position corresponding to position 377; H at a position corresponding to position 379; S at a position corresponding to position 379; T at a position corresponding to position 379; I at a position corresponding to position 380; L at a position corresponding to position 380; P at a position corresponding to position 380; T at a position corresponding to position 380; H at a position corresponding to position 388; N at a position corresponding to position 406; F at a position corresponding to position 407; Q at a position corresponding to position 407; S at a position corresponding to position 410; G at a position corresponding to position 412; P at a position corresponding to position 412; S at a position corresponding to position 412; Q at a position corresponding to position 413; M at a position corresponding to position 421; P at a position corresponding to position 428; A at a position corresponding to position 431; L at a position corresponding to position 433; T at a position corresponding to position 433; A at a position corresponding to position 438; C at a position corresponding to position 439; T at a position corresponding to position 441; M at a position corresponding to position 443; Y at a position corresponding to position 445; C at a position corresponding to position 446; D at a position corresponding to position 446; E at a position corresponding to position 446; G at a position corresponding to position 446; E at a position corresponding to position 447; or G at a position corresponding to position 447, with reference to amino acid residue positions of the sequence set forth in SEQ ID NO:3.

In examples of any of the modified PH20 polypeptides provided herein, the modified PH20 polypeptide contains only one amino acid replacement compared to the unmodified PH20 polypeptide. In other examples of any of the modified PH20 polypeptides provided herein, the modified PH20 polypeptide contains at least 2, 3, 4, 5, 6, 7, 8, 9, 10, 11, 12, 13, 14, 15, 16, 17, 18, 19 or 20 amino acid replacements compared to the unmodified PH20 polypeptide or contains 2, 3, 4, 5, 6, 7, 8, 9, 10, 11, 12, 13, 14, 15, 16, 17, 18, 19 or 20 amino acid replacements compared to the unmodified PH20 polypeptide.

For example, included among modified PH20 polypeptides provided herein are any that contain at least 2 amino acid replacements, such as at least 3, 4, 5, 6, 7, 8, 9, 10, 11, 12, 13, 14, 15, 16, 17, 18, 19 or 20 amino acid replacements, where the amino acid replacements are two or more of replacement with: glycine (G) at a position corresponding to position 11; A at a position corresponding to position 15; V at a position corresponding to position 15; R at a position corresponding to position 26; S at a position corresponding to position 26; E at a position corresponding to position 27; H at a position corresponding to position 27; H at a position corresponding to position 29; S at a position corresponding to position 29; A at a position corresponding to position 30; P at a position corresponding to position 30; G at a position corresponding to position 31; L at a position corresponding to position 31; Q at a position corresponding to position 32; W at a position corresponding to position 32; G at a position corresponding to position 33; M at a position corresponding to position 33; R at a position corresponding to position 33; W at a position corresponding to position 33; E at a position corresponding to position 34; H at a position corresponding to position 34; W at a position corresponding to position 34; K at a position corresponding to position 37; Y at a position corresponding to position 38; Q at a position corresponding to position 39; R at a position corresponding to position 39; T at a position corresponding to position 39; D at a position corresponding to position 41; T at a position corresponding to position 41; W at a position corresponding to position 41; G at a position corresponding to position 48; C at a position corresponding to position 50; D at a position corresponding to position 50; K at a position corresponding to position 58; R at a position corresponding to position 58; K at a position corresponding to position 60; F at a position corresponding to position 67; A at a position corresponding to position 69; Y at a position corresponding to position 69; Q at a position corresponding to position 83; D at a position corresponding to position 84; D at a position corresponding to position 86; E at a position corresponding to position 86; R at a position corresponding to position 86; P at a position corresponding to position 87; W at a position corresponding to position 90; V at a position corresponding to position 92; E at a position corresponding to position 93; S at a position corresponding to position 93; N at a position corresponding to position 94; F at a position corresponding to position 97; M at a position corresponding to position 98; S at a position corresponding to position 99; H at a position corresponding to position 102; G at a position corresponding to position 114; M at a position corresponding to position 118; S at a position corresponding to position 120; C at a position corresponding to position 131; L at a position corresponding to position 131; A at a position corresponding to position 132; W at a position corresponding to position 138; R at a position corresponding to position 139; V at a position corresponding to position 139; M at a position corresponding to position 141; Y at a position corresponding to position 141; K at a position corresponding to position 143; G at a position corresponding to position 144; V at a position corresponding to position 146; I at a position corresponding to position 147; M at a position corresponding to position 147; C at a position corresponding to position 148; H at a position corresponding to position 148; K at a position corresponding to position 148; L at a position corresponding to position 150; Q at a position corresponding to position 151; I at a position corresponding to position 152; M at a position corresponding to position 152; T at a position corresponding to position 152; R at a position corresponding to position 154; A at a position corresponding to position 155; D at a position corresponding to position 155; F at a position corresponding to position 155; H at a position corresponding to position 155; L at a position corresponding to position 155; R at a position corresponding to position 155; S at a position corresponding to position 155; H at a position corresponding to position 158; A at a position corresponding to position 159; H at a position corresponding to position 159; N at a position corresponding to position 159; Q at a position corresponding to position 159; S at a position corresponding to position 159; Y at a position corresponding to position 160; A at a position corresponding to position 161; L at a position corresponding to position 162; K at a position corresponding to position 163; R at a position corresponding to position 163; S at a position corresponding to position 163; F at a position corresponding to position 165; W at a position corresponding to position 174; H at a position corresponding to position 195; L at a position corresponding to position 195; T at a position corresponding to position 196; F at a position corresponding to position 197; L at a position corresponding to position 198; P at a position corresponding to position 204; A at a position corresponding to position 205; E at a position corresponding to position 205; K at a position corresponding to position 205; L at a position corresponding to position 205; T at a position corresponding to position 205; I at a position corresponding to position 206; Q at a position corresponding to position 208; R at a position corresponding to position 208; E at a position corresponding to position 213; N at a position corresponding to position 213; E at a position corresponding to position 215; H at a position corresponding to position 215; T at a position corresponding to position 215; N at a position corresponding to position 222; T at a position corresponding to position 235; Q at a position corresponding to position 237; Q at a position corresponding to position 240; I at a position corresponding to position 247; L at a position corresponding to position 251; M at a position corresponding to position 251; K at a position corresponding to position 259; P at a position corresponding to position 259; M at a position corresponding to position 260; A at a position corresponding to position 261; F at a position corresponding to position 261; T at a position corresponding to position 263; Vat a position corresponding to position 271; E at a position corresponding to position 276; A at a position corresponding to position 277; C at a position corresponding to position 277; N at a position corresponding to position 278; Q at a position corresponding to position 282; A at a position corresponding to position 284; Q at a position corresponding to position 284; S at a position corresponding to position 284; M at a position corresponding to position 285; V at a position corresponding to position 292; N at a position corresponding to position 305; D at a position corresponding to position 306; R at a position corresponding to position 310; G at a position corresponding to position 311; T at a position corresponding to position 315; N at a position corresponding to position 317; A at a position corresponding to position 321; R at a position corresponding to position 321; L at a position corresponding to position 328; R at a position corresponding to position 328; A at a position corresponding to position 342; H at a position corresponding to position 368; K at a position corresponding to position 368; H at a position corresponding to position 369; F at a position corresponding to position 371; S at a position corresponding to position 373; T at a position corresponding to position 377; H at a position corresponding to position 379; S at a position corresponding to position 379; T at a position corresponding to position 379; I at a position corresponding to position 380; L at a position corresponding to position 380; P at a position corresponding to position 380; T at a position corresponding to position 380; H at a position corresponding to position 388; N at a position corresponding to position 406; F at a position corresponding to position 407; Q at a position corresponding to position 407; S at a position corresponding to position 410; G at a position corresponding to position 412; P at a position corresponding to position 412; S at a position corresponding to position 412; Q at a position corresponding to position 413; M at a position corresponding to position 421; P at a position corresponding to position 428; A at a position corresponding to position 431; L at a position corresponding to position 433; T at a position corresponding to position 433; A at a position corresponding to position 438; C at a position corresponding to position 439; T at a position corresponding to position 441; M at a position corresponding to position 443; Y at a position corresponding to position 445; C at a position corresponding to position 446; D at a position corresponding to position 446; E at a position corresponding to position 446; G at a position corresponding to position 446; E at a position corresponding to position 447; or G at a position corresponding to position 447, with reference to amino acid residue positions of the sequence set forth in SEQ ID NO:3.

In any of the above examples of a modified PH20 polypeptide provided herein, the amino acid replacement or amino acid replacements include replacement with: alanine (A) at a position corresponding to position 15; V at a position corresponding to position 15; R at a position corresponding to position 26; E at a position corresponding to position 27; S at a position corresponding to position 29; G at a position corresponding to position 31; L at a position corresponding to position 31; Q at a position corresponding to position 32; G at a position corresponding to position 33; M at a position corresponding to position 33; R at a position corresponding to position 33; W at a position corresponding to position 33; E at a position corresponding to position 34; H at a position corresponding to position 34; Y at a position corresponding to position 38; R at a position corresponding to position 39; W at a position corresponding to position 41; G at a position corresponding to position 48; C at a position corresponding to position 50; R at a position corresponding to position 58; A at a position corresponding to position 69; D at a position corresponding to position 86; E at a position corresponding to position 86; R at a position corresponding to position 86; W at a position corresponding to position 90; E at a position corresponding to position 93; S at a position corresponding to position 93; F at a position corresponding to position 97; S at a position corresponding to position 99; S at a position corresponding to position 120; L at a position corresponding to position 131; A at a position corresponding to position 132; W at a position corresponding to position 138; R at a position corresponding to position 139; V at a position corresponding to position 139; M at a position corresponding to position 141; Y at a position corresponding to position 141; K at a position corresponding to position 143; V at a position corresponding to position 146; I at a position corresponding to position 147; M at a position corresponding to position 147; C at a position corresponding to position 148; H at a position corresponding to position 148; K at a position corresponding to position 148; L at a position corresponding to position 150; Q at a position corresponding to position 151; I at a position corresponding to position 152; M at a position corresponding to position 152; T at a position corresponding to position 152; R at a position corresponding to position 154; A at a position corresponding to position 155; F at a position corresponding to position 155; L at a position corresponding to position 155; R at a position corresponding to position 155; H at a position corresponding to position 158; H at a position corresponding to position 159; N at a position corresponding to position 159; Q at a position corresponding to position 159; S at a position corresponding to position 159; Y at a position corresponding to position 160; R at a position corresponding to position 163; F at a position corresponding to position 165; W at a position corresponding to position 174; L at a position corresponding to position 198; P at a position corresponding to position 204; A at a position corresponding to position 205; L at a position corresponding to position 205; T at a position corresponding to position 205; I at a position corresponding to position 206; Q at a position corresponding to position 208; R at a position corresponding to position 208; N at a position corresponding to position 213; E at a position corresponding to position 215; T at a position corresponding to position 215; T at a position corresponding to position 235; Q at a position corresponding to position 237; Q at a position corresponding to position 240; L at a position corresponding to position 251; K at a position corresponding to position 259; M at a position corresponding to position 260; A at a position corresponding to position 261; F at a position corresponding to position 261; T at a position corresponding to position 263; Vat a position corresponding to position 271; E at a position corresponding to position 276; A at a position corresponding to position 277; C at a position corresponding to position 277; A at a position corresponding to position 284; Q at a position corresponding to position 284; S at a position corresponding to position 284; V at a position corresponding to position 292; N at a position corresponding to position 305; D at a position corresponding to position 306; R at a position corresponding to position 310; T at a position corresponding to position 315; R at a position corresponding to position 328; A at a position corresponding to position 342; K at a position corresponding to position 368; H at a position corresponding to position 369; S at a position corresponding to position 373; H at a position corresponding to position 379; S at a position corresponding to position 379; T at a position corresponding to position 379; I at a position corresponding to position 380; L at a position corresponding to position 380; P at a position corresponding to position 380; T at a position corresponding to position 380; H at a position corresponding to position 388; G at a position corresponding to position 412; P at a position corresponding to position 412; S at a position corresponding to position 412; Q at a position corresponding to position 413; T at a position corresponding to position 433; A at a position corresponding to position 438; T at a position corresponding to position 441; M at a position corresponding to position 443; Y at a position corresponding to position 445; C at a position corresponding to position 446; E at a position corresponding to position 447; or G at a position corresponding to position 447, with reference to positions in SEQ ID NO: 3.

For example, in any of the above examples of a modified PH20 polypeptide provided herein, the amino acid replacement or amino acid replacements include replacement with: alanine (A) at a position corresponding to position 15; V at a position corresponding to position 15; R at a position corresponding to position 26; E at a position corresponding to position 27; S at a position corresponding to position 29; G at a position corresponding to position 31; G at a position corresponding to position 33; M at a position corresponding to position 33; R at a position corresponding to position 33; W at a position corresponding to position 33; E at a position corresponding to position 34; H at a position corresponding to position 34; Y at a position corresponding to position 38; R at a position corresponding to position 39; G at a position corresponding to position 48; R at a position corresponding to position 86; W at a position corresponding to position 90; E at a position corresponding to position 93; S at a position corresponding to position 93; F at a position corresponding to position 97; S at a position corresponding to position 120; L at a position corresponding to position 131; A at a position corresponding to position 132; R at a position corresponding to position 139; M at a position corresponding to position 141; Y at a position corresponding to position 141; K at a position corresponding to position 143; I at a position corresponding to position 147; M at a position corresponding to position 147; C at a position corresponding to position 148; H at a position corresponding to position 148; K at a position corresponding to position 148; M at a position corresponding to position 152; T at a position corresponding to position 152; R at a position corresponding to position 154; A at a position corresponding to position 155; F at a position corresponding to position 155; L at a position corresponding to position 155; N at a position corresponding to position 159; S at a position corresponding to position 159; Y at a position corresponding to position 160; R at a position corresponding to position 163; F at a position corresponding to position 165; W at a position corresponding to position 174; L at a position corresponding to position 198; P at a position corresponding to position 204; A at a position corresponding to position 205; L at a position corresponding to position 205; T at a position corresponding to position 205; I at a position corresponding to position 206; R at a position corresponding to position 208; N at a position corresponding to position 213; E at a position corresponding to position 215; T at a position corresponding to position 215; Q at a position corresponding to position 240; L at a position corresponding to position 251; K at a position corresponding to position 259; M at a position corresponding to position 260; A at a position corresponding to position 261; F at a position corresponding to position 261; T at a position corresponding to position 263; V at a position corresponding to position 271; A at a position corresponding to position 277; C at a position corresponding to position 277; A at a position corresponding to position 284; Q at a position corresponding to position 284; S at a position corresponding to position 284; V at a position corresponding to position 292; T at a position corresponding to position 315; A at a position corresponding to position 342; H at a position corresponding to position 369; H at a position corresponding to position 379; S at a position corresponding to position 379; T at a position corresponding to position 379; L at a position corresponding to position 380; P at a position corresponding to position 380; T at a position corresponding to position 380; H at a position corresponding to position 388; G at a position corresponding to position 412; P at a position corresponding to position 412; S at a position corresponding to position 412; T at a position corresponding to position 433; A at a position corresponding to position 438; T at a position corresponding to position 441; M at a position corresponding to position 443; Y at a position corresponding to position 445; C at a position corresponding to position 446; E at a position corresponding to position 447; or G at a position corresponding to position 447, with reference to positions in SEQ ID NO: 3.

In another example, in any of the above examples of a modified PH20 polypeptide provided herein, the amino acid replacement or amino acid replacements include replacement with: glutamic acid (E) at a position corresponding to position 27; A at a position corresponding to position 132; K at a position corresponding to position 143; M at a position corresponding to position 147; C at a position corresponding to position 148; H at a position corresponding to position 148; Y at a position corresponding to position 160; P at a position corresponding to position 204; A at a position corresponding to position 205; I at a position corresponding to position 206; T at a position corresponding to position 215; M at a position corresponding to position 260; A at a position corresponding to position 261; F at a position corresponding to position 261; T at a position corresponding to position 263; A at a position corresponding to position 284; T at a position corresponding to position 315; and S at a position corresponding to position 379, with reference to positions in SEQ ID NO: 3.

In a further example, in any of the above examples of a modified PH20 polypeptide provided herein, the amino acid replacement or amino acid replacements include replacement with: P at a position corresponding to position 30; R at a position corresponding to position 58; K at a position corresponding to position 60; K at a position corresponding to position 143; I at a position corresponding to position 147; P at a position corresponding to position 204; T at a position corresponding to position 215; T at a position corresponding to position 235; A at a position corresponding to position 261; G at a position corresponding to position 311; T at a position corresponding to position 315; or H at a position corresponding to position 369, with reference to positions in SEQ ID NO: 3.

In examples herein, in any of the above examples of a modified PH20 polypeptide provided herein, the amino acid replacement or amino acid replacements include replacement with: P at a position corresponding to position 30; K at a position corresponding to position 60; I at a position corresponding to position 147; T at a position corresponding to position 215; T at a position corresponding to position 235; G at a position corresponding to position 311; T at a position corresponding to position 315; or H at a position corresponding to position 369, with reference to positions in SEQ ID NO: 3.

In any of the above examples, the modified PH20 polypeptide contain an amino acid replacement to amino acid replacements in an unmodified PH20 polypeptide that has the sequence of amino acids set forth in any of SEQ ID NOS: 3, 7, 10, 12, 14, 24, 32-66, 69, 72, 388, 390, 392, or 400 or a sequence of amino acids that is at least 80%, 85%, 86%, 87%, 88%, 89%, 90%, 91%, 92%, 93%, 94%, 95%, 96%, 97%, 98%, 99% identical to any of SEQ ID NOS: 3, 7, 10, 12, 14, 24, 32-66, 69, 72, 388, 390, 392, or 400. For example, the amino acid replacement or replacements is/are in an unmodified PH20 polypeptide that has the sequence of amino acids set forth in SEQ ID NOS: 3, 7, 32-66, 69 or 72, or a sequence of amino acids that exhibits at least 91% sequence identity to any of SEQ ID NOS: 3, 7, 32-66, 69 or 72. The unmodified polypeptide can be a human polypeptide.

In any of the examples of a modified PH20 polypeptide provided herein, the modified PH20 polypeptide exhibits at least 68% amino acid sequence identity to the sequence of amino acids set forth in SEQ ID NO:3, such as at least 70%, 75%, 80%, 85%, 86%, 87%, 88%, 89%, 90%, 91%, 92%, 93%, 94%, 95%, 96%, 97%, 98% or 99% amino acid sequence identity to the sequence of amino acids set forth in SEQ ID NO:3. Included among any of the modified PH20 polypeptides provided herein are modified PH20 polypeptides that are a mature PH20 polypeptide lacking the signal sequence. In examples herein, the modified PH20 polypeptide does not contain or consist of the sequence of amino acids set forth in any of SEQ ID NOS: 8-31, 69, 72, 387-392, 399 or 400.

For example, provided herein are a modified PH20 polypeptide containing the sequence of amino acids set forth in any of SEQ ID NOS: 73-386 or a sequence of amino acids that exhibits at least 85%, 90%, 91%, 92%, 93%, 94%, 95%, 96%, 97%, 98%, 99% or more sequence identity to a sequence of amino acids set forth in any of SEQ ID NOS: 73-386.

In any of the examples of a modified PH20 polypeptides provided herein, the modified PH20 polypeptide is substantially purified or isolated. Any of the modified PH20 polypeptides provided herein exhibit hyaluronidase activity at neutral pH. Any of the modified PH20 polypeptides provided herein include those that are capable of being secreted upon expression from cells and that are soluble in the supernatant. For example, the cell can be a mammalian cell, such as BHK, CHO, Balb/3T3, HeLa, MT2, mouse NS0 (nonsecreting) and other myeloma cell lines, hybridoma and heterohybridoma cell lines, lymphocytes, fibroblasts, Sp2/0, COS, NIH3T3, HEK293, 293S, 2B8, or HKB cells.

Included among any of the modified PH20 polypeptides provided herein are any that are modified by or contain one or more of glycosylation, sialation, albumination, farnysylation, carboxylation, hydroxylation or phosphorylation. For example, the modified PH20 polypeptide is glycosylated, whereby the polypeptide has at least an N-acetylglucosamine moiety linked to each of at least three asparagine (N) residues, such as asparagine residues that correspond to amino acid residues 200, 333 and 358 of SEQ ID NO:3.

Also included among any of the modified PH20 polypeptides provided herein are any that are conjugated to a polymer, such as a dextran or PEG or to a moiety that is a multimerization domain, toxin, detectable label or drug. For example, the modified PH20 polypeptide is conjugated to an Fc domain. Also provided herein are conjugates containing any of the modified PH20 polypeptides provided herein linked directly or indirectly via a linker to a targeting agent.

Provided herein are nucleic acid molecules encoding any of the modified PH20 polypeptide provided herein. Also provided are vectors containing any of the nucleic acids provided herein. The vector can be eukaryotic or a prokaryotic vector, such as a mammalian vector or a viral vector. For example, the vector is an adenovirus vector, a retrovirus vector or a vaccinia virus vector. Also provided herein are cells containing any of the vectors provided herein. The cell can be a mammalian cell or non-mammalian cell. For example, the cell is a mammalian cell, such as a Chinese Hamster Ovary (CHO) cell.

Provided herein is a method of producing a modified PH20 polypeptide that is an uber-thermophile by introducing any of the nucleic acids or vectors provided herein into a cell capable of incorporating N-linked sugar moieties into the polypeptide, culturing the cell under conditions whereby an encoded modified PH20 polypeptide is produced and secreted by the cell; and recovering the expressed PH20 polypeptide. In such a method, the nucleic acid is operably linked to a promoter. The cell can be a eukaryotic cell or a prokaryotic cell. Typically, the cell is a cell capable of glycosylation. For example, the cell is a mammalian cell, such as a Chinese hamster ovary (CHO) cell. Also provided herein are modified PH20 polypeptides produced by the above method.

Provided herein are pharmaceutical compositions containing any of the modified PH20 polypeptides provided herein. The pharmaceutical composition can contain a pharmaceutically acceptable excipient. The modified PH20 polypeptide in the any of the pharmaceutical compositions provided herein exhibits greater than 75%, 80%, 85%, 90%, 95% or more of its hyaluronidase when stored without refrigeration for greater than 48 hours compared to when it stored with refrigeration for the same time period. For example, the activity is exhibited when stored without refrigeration for greater than 72 hours, 96 hours, one week, two weeks, three weeks, one month, two months, three months, four months, five months or six months compared to when it is stored with refrigeration for the same time period. In such examples, storing the composition without refrigeration exposes the composition to an ambient temperature that is between 18° C. to 45° C., 25° C. to 42° C. or 30° C. to 37° C. for the time period, including time periods that are continuous, intermittent or variable.

In any of the examples of a pharmaceutical composition provided herein, the pharmaceutical composition is formulated in the absence of a stabilizer that is an amino acid, an amino acid derivative, an amine, a sugar, a polyol, a surfactant, a preservative, a hyaluronidase inhibitor or an albumin protein. For example, the pharmaceutical composition is formulated in the absence of salt or is formulated with a concentration of salt that is less than 130 mM.

In any of the examples of a pharmaceutical composition provided herein, the pharmaceutical composition is formulated for single dose administration or multiple dose administration. The pharmaceutical composition can be formulated for direct administration. Included among any of the pharmaceutical compositions provided herein are liquid compositions.

In examples of any of the pharmaceutical compositions provided herein, the concentration of modified PH20 is from or from about 0.1 μg/mL to 100 μg/mL, 1 μg/mL to 50 μg/mL or 1 μg/mL to 20 μg/mL. For example, the amount of a modified PH20 in any of the pharmaceutical compositions provided herein is between or about between 10 U/mL to 5000 U/mL, 50 U/mL to 4000 U/mL, 100 U/mL to 2000 U/mL, 300 U/mL to 2000 U/mL, 600 U/mL to 2000 U/mL, or 100 U/mL to 1000 U/mL. The volume of any of the pharmaceutical compositions provided herein is from or from about 0.5 mL to 50 mL, 1 mL to 10 mL, or 1 mL to 5 mL, for example at least 0.5 mL, 1 mL, 2 mL, 3 mL, 4 mL, 5 mL, 6 mL, 7 mL, 8 mL, 9 mL, 10 mL, 15 mL, 20 mL, 25 mL, 30 mL, 35 mL, 40 mL, 45 mL, 50 mL or more.

Included among any of the pharmaceutical compositions provided herein are any that contain any of the modified PH20 polypeptides provided herein and a therapeutically active agent. Also provided herein are combinations containing a first composition that is any of the above compositions provided herein or is any composition that contains a modified PH20 polypeptide provided herein, and a second composition that contains a therapeutic agent or therapeutically active agent. In any of the above compositions or combinations, the therapeutic agent is a polypeptide, a protein, a nucleic acid, a drug, a small molecule or an organic molecule. For example, the therapeutically active agent is a chemotherapeutic agent, an analgesic agent, an anti-inflammatory agent, an antimicrobial agent, an amoebicidal agent, a trichomonocidal agent, an anti-Parkinson agent, an anti-malarial agent, an anticonvulsant agent, an anti-depressant agent, and antiarthritics agent, an anti-fungal agent, an antihypertensive agent, an antipyretic agent, an anti-parasite agent, an antihistamine agent, an alpha-adrenargic agonist agent, an alpha blocker agent, an anesthetic agent, a bronchial dilator agent, a biocide agent, a bactericide agent, a bacteriostat agent, a beta adrenergic blocker agent, a calcium channel blocker agent, a cardiovascular drug agent, a contraceptive agent, a decongestant agent, a diuretic agent, a depressant agent, a diagnostic agent, an electrolyte agent, a hypnotic agent, a hormone agent, a hyperglycemic agent, a muscle relaxant agent, a muscle contractant agent, an ophthalmic agent, a parasympathomimetic agent, a psychic energizer agent, a sedative agent, a sympathomimetic agent, a tranquilizer agent, a urinary agent, a vaginal agent, a viricide agent, a vitamin agent, a non-steroidal anti-inflammatory agent, an angiotensin converting enzyme inhibitor agent, or a sleep inducer.

For example, included in any of the above compositions or combinations, the therapeutic agent is an antibody, an Immune Globulin, a bisphosphonate, a cytokine, a chemotherapeutic agent, a coagulation factor or an insulin, such as a fast-acting insulin. Also included in any of the above compositions or combinations, the therapeutic agent is Adalimumabs, Agalsidase Betas, Alefacepts, Ampicillins, Anakinras, Antipoliomyelitic Vaccines, Anti-Thymocytes, Azithromycins, Becaplermins, Caspofungins, Cefazolins, Cefepimes, Cefotetans, Ceftazidimes, Ceftriaxones, Cetuximabs, Cilastatins, Clavulanic Acids, Clindamycins, Darbepoetin Alfas, Daclizumabs, Diphtheria, Diphtheria antitoxins, Diphtheria Toxoids, Efalizumabs, Epinephrines, Erythropoietin Alphas, Etanercepts, Filgrastims, Fluconazoles, Follicle-Stimulating Hormones, Follitropin Alphas, Follitropin Betas, Fosphenytoins, Gadodiamides, Gadopentetates, Gatifloxacins, Glatiramers, Granulocyte macrophage colony-stimulating factors (GM-CSFs), Goserelin acetates, Granisetrons, Haemophilus Influenza Bs, Haloperidols, Hepatitis vaccines, Hepatitis A Vaccines, Hepatitis B Vaccines, Ibritumomab Tiuxetans, Ibritumomabs, Tiuxetans, Immunoglobulins, Hemophilus influenza vaccines, Influenza Virus Vaccines, Infliximabs, Insulin lispro, 75% neutral protamine lispro (NPL)/25% insulin lispro, 50% neutral protamine Hagedorn (NPH)/50% regular insulin, 70% NPH/30% regular insulin, Regular insulin, NPH insulin, Ultra insulin, Ultralente insulin, Insulin Glargines, Interferons, Interferon alphas, Interferon betas, Interferon gammas, Interferon alpha-2a, Interferon alpha-2b, Interferon Alphacon, Interferon alpha-n, Interferon Betas, Interferon Beta-1as, Interferon Gammas, Interferon alpha-con, Iodixanols, Iohexols, Iopamidols, Ioversols, Ketorolacs, Laronidases, Levofloxacins, Lidocaines, Linezolids, Lorazepams, Measles Vaccines, Measles virus, Mumps viruses, Measles-Mumps-Rubella Virus Vaccines, Rubella vaccines, Medroxyprogesterones, Meropenems, Methylprednisolones, Midazolams, Morphines, Octreotides, Omalizumabs, Ondansetrons, Palivizumabs, Pantoprazoles, Pegaspargases, Pegfilgrastims, Peg-Interferon Alpha-2as, Peg-Interferon Alpha-2bs, Pegvisomants, Pertussis vaccines, Piperacillins, Pneumococcal Vaccines Pneumococcal Conjugate Vaccines, Promethazines, Reteplases, Somatropins, Sulbactams, Sumatriptans, Tazobactams, Tenecteplases, Tetanus Purified Toxoids, Ticarcillins, Tositumomabs, Triamcinolones, Triamcinolone Acetonides, Triamcinolone hexacetonides, Vancomycins, Varicella Zoster immunoglobulins, Varicella vaccines, other vaccines, Alemtuzumabs, Alitretinoins, Allopurinols, Altretamines, Amifostines, Anastrozoles, Arsenics, Arsenic Trioxides, Asparaginases, Bacillus Calmette-Guerin (BCG) vaccines, BCG Live, Bexarotenes, Bleomycins, Busulfans, Busulfan intravenous, Busulfan orals, Calusterones, Capecitabines, Carboplatins, Carmustines, Carmustines with Polifeprosans, Celecoxibs, Chlorambucils, Cisplatins, Cladribines, Cyclophosphamides, Cytarabines, Cytarabine liposomals, Dacarbazines, Dactinomycins, Daunorubicin liposomals, Daunorubicins, Denileukin Diftitoxes, Dexrazoxanes, Docetaxels, Doxorubicins, Doxorubicin liposomals, Dromostanolone propionates, Elliotts B Solutions, Epirubicins, Epoetin alfas, Estramustines, Etoposide phosphates, Exemestanes, Floxuridines, Fludarabines, Fluorouracils, Fulvestrants, Gemcitabines, Gemtuzumabs, Ozogamicins, Gemtuzumab ozogamicins, Hydroxyureas, Idarubicins, Ifosfamides, Imatinib mesylates, Irinotecans, Letrozoles, Leucovorins, Levamisoles, Lomustines, Mechlorethamines, Nitrogen mustards, Megestrols, Megestrol acetates, Melphalans, Mercaptopurines, Mesnas, Methotrexates, Methoxsalens, Mitomycins, Mitomycin Cs, Mitotanes, Mitoxantrones, Nandrolones, Nandrolone Phenpropionates, Nofetumomabs, Oprelvekins, Oxaliplatins, Paclitaxels, Pamidronates, Pegademases, Pentostatins, Pipobromans, Plicamycins, Porfimer sodiums, Procarbazines, Quinacrines, Rasburicases, Rituximabs, Sargramostims, Streptozocins, Talcs, Tamoxifens, Temozolomides, Teniposides, Testolactones, Thioguanines, Triethylenethiophosphoramides (Thiotepas), Topotecans, Toremifenes, Trastuzumabs, Tretinoins, Uracil Mustards, Valrubicins, Vinblastines, Vincristines, Vinorelbines, Zoledronates, Acivicins, Aclarubicins, Acodazoles, Acronines, Adozelesins, Retinoic Acids, 9-Cis-Retinoic Acids, Alvocidibs, Ambazones, Ambomycins, Ametantrones, Aminoglutethimides, Amsacrines, Anaxirones, Ancitabines, Anthramycins, Apaziquones, Argimesnas, Asperlins, Atrimustines, Azacitidines, Azetepas, Azotomycins, Banoxantrones, Batabulins, Batimastats, Benaxibines, Bendamustines, Benzodepas, Bicalutamides, Bietaserpines, Biricodars, Bisantrenes, Bisnafide Dimesylates, Bizelesins, Bortezomibs, Brequinars, Bropirimines, Budotitanes, Cactinomycins, Canertinibs, Caracemides, Carbetimers, Carboquones, Carmofurs, Carubicins, Carzelesins, Cedefingols, Cemadotins, Cioteronels, Cirolemycins, Clanfenurs, Clofarabines, Crisnatols, Decitabines, Dexniguldipines, Dexormaplatins, Dezaguanines, Diaziquones, Dibrospidiums, Dienogests, Dinalins, Disermolides, Dofequidars, Doxifluridines, Droloxifenes, Duazomycins, Ecomustines, Edatrexates, Edotecarins, Eflomithines, Elacridars, Elinafides, Elsamitrucins, Emitefurs, Enloplatins, Enpromates, Enzastaurins, Epipropidines, Eptaloprosts, Erbulozoles, Esorubicins, Etanidazoles, Etoglucids, Etoprines, Exisulinds, Fadrozoles, Fazarabines, Fenretinides, Fluoxymesterones, Flurocitabines, Fosquidones, Fostriecins, Fotretamines, Galarubicins, Galocitabines, Geroquinols, Gimatecans, Gimeracils, Gloxazones, Glufosfamides, Ilmofosines, Ilomastats, Imexons, Improsulfans, Indisulams, Inproquones, Interleukins, Interleukin-2s, recombinant Interleukins, Intoplicines, Iobenguanes, Iproplatins, Irsogladines, Ixabepilones, Ketotrexates, L-Alanosines, Lanreotides, Lapatinibs, Ledoxantrones, Leuprolides, Lexacalcitols, Liarozoles, Lobaplatins, Lometrexols, Lonafarnibs, Losoxantrones, Lurtotecans, Mafosfamides, Mannosulfans, Marimastats, Masoprocols, Maytansines, Melengestrols, Menogarils, Mepitiostanes, Metesinds, Metomidates, Metoprines, Meturedepas, Miboplatins, Miproxifenes, Misonidazoles, Mitindomides, Mitocarcins, Mitocromins, Mitoflaxones, Mitogillins, Mitoguazones, Mitomalcins, Mitonafides, Mitoquidones, Mitospers, Mitozolomides, Mivobulins, Mizoribines, Mofarotenes, Mopidamols, Mubritinibs, Mycophenolic Acids, Nedaplatins, Nelarabines, Nemorubicins, Nitracrines, Nocodazoles, Nogalamycins, Nolatrexeds, Nortopixantrones, Ormaplatins, Ortataxels, Oteracils, Oxisurans, Oxophenarsines, Patupilones, Peldesines, Peliomycins, Pelitrexols, Pemetrexeds, Pentamustines, Peplomycins, Perfosfamides, Perifosines, Picoplatins, Pinafides, Piposulfans, Pirfenidones, Piroxantrones, Pixantrones, Plevitrexeds, Plomestanes, Porfiromycins, Prednimustines, Propamidines, Prospidiums, Pumitepas, Puromycins, Pyrazofurins, Ranimustines, Riboprines, Ritrosulfans, Rogletimides, Roquinimexs, Sabarubicins, Safingols, Satraplatins, Sebriplatins, Semustines, Simtrazenes, Sizofirans, Sobuzoxanes, Sorafenibs, Sparfosates, Sparfosic Acids, Sparsomycins, Spirogermaniums, Spiromustines, Spiroplatins, Squalamines, Streptonigrins, Streptovarycins, Sufosfamides, Sulofenurs, Tacedinalines, Talisomycins, Tallimustines, Tariquidars, Tauromustines, Tecogalans, Tegafurs, Teloxantrones, Temoporfins, Teroxirones, Thiamiprines, Tiamiprines, Tiazofurins, Tilomisoles, Tilorones, Timcodars, Timonacics, Tirapazamines, Topixantrones, Trabectedins, Trestolones, Triciribines, Trilostanes, Trimetrexates, Triplatin Tetranitrates, Triptorelins, Trofosfamides, Tubulozoles, Ubenimexs, Uredepas, Valspodars, Vapreotides, Verteporfins, Vindesines, Vinepidines, Vinflunines, Vinformides, Vinglycinates, Vinleucinols, Vinleurosines, Vinrosidines, Vintriptols, Vinzolidines, Vorozoles, Xanthomycin As, Guamecyclines, Zeniplatins, Zilascorbs [2-H], Zinostatins, Zorubicins, Zosuquidars, Acetazolamides, Acyclovirs, Adipiodones, Alatrofloxacins, Alfentanils, Allergenic extracts, Alpha 1-proteinase inhibitors, Alprostadils, Amikacins, Amino acids, Aminocaproic acids, Aminophyllines, Amitriptylines, Amobarbitals, Amrinones, Analgesics, Anti-poliomyelitis vaccines, Anti-rabic serums, Anti-tetanus immunoglobulins, tetanus vaccines, Antithrombin IIIs, Antivenom serums, Argatrobans, Arginines, Ascorbic acids, Atenolols, Atracuriums, Atropines, Aurothioglucoses, Azathioprines, Aztreonams, Bacitracins, Baclofens, Basiliximabs, Benzoic acids, Benztropines, Betamethasones, Biotins, Bivalirudins, Botulism antitoxins, Bretyliums, Bumetanides, Bupivacaines, Buprenorphines, Butorphanols, Calcitonins, Calcitriols, Calciums, Capreomycins, Carboprosts, Carnitines, Cefamandoles, Cefoperazones, Cefotaximes, Cefoxitins, Ceftizoximes, Cefuroximes, Chloramphenicols, Chloroprocaines, Chloroquines, Chlorothiazides, Chlorpromazines, Chondroitinsulfuric acids, Choriogonadotropin alfas, Chromiums, Cidofovirs, Cimetidines, Ciprofloxacins, Cisatracuriums, Clonidines, Codeines, Colchicines, Colistins, Collagens, Corticorelin ovine triflutates, Corticotrophins, Cosyntropins, Cyanocobalamins, Cyclosporines, Cysteines, Dacliximabs, Dalfopristins, Dalteparins, Danaparoids, Dantrolenes, Deferoxamines, Desmopressins, Dexamethasones, Dexmedetomidines, Dexpanthenols, Dextrans, Iron dextrans, Diatrizoic acids, Diazepams, Diazoxides, Dicyclomines, Digibinds, Digoxins, Dihydroergotamines, Diltiazems, Diphenhydramines, Dipyridamoles, Dobutamines, Dopamines, Doxacuriums, Doxaprams, Doxercalciferols, Doxycyclines, Droperidols, Dyphyllines, Edetic acids, Edrophoniums, Enalaprilats, Ephedrines, Epoprostenols, Ergocalciferols, Ergonovines, Ertapenems, Erythromycins, Esmolols, Estradiols, Estrogenics, Ethacrynic acids, Ethanolamines, Ethanols, Ethiodized oils, Etidronic acids, Etomidates, Famotidines, Fenoldopams, Fentanyls, Flumazenils, Fluoresceins, Fluphenazines, Folic acids, Fomepizoles, Fomivirsens, Fondaparinuxs, Foscarnets, Fosphenytoins, Furosemides, Gadoteridols, Gadoversetamides, Ganciclovirs, Gentamicins, Glucagons, Glucoses, Glycines, Glycopyrrolates, Gonadorelins, Gonadotropin chorionics, Haemophilus B polysaccharides, Hemins, Herbals, Histamines, Hydralazines, Hydrocortisones, Hydromorphones, Hydroxocobalamins, Hydroxyzines, Hyoscyamines, Ibutilides, Imiglucerases, Indigo carmines, Indomethacins, Iodides, Iopromides, Iothalamic acids, Ioxaglic acids, Ioxilans, Isoniazids, Isoproterenols, Japanese encephalitis vaccines, Kanamycins, Ketamines, Labetalols, Lepirudins, Levobupivacaines, Levothyroxines, Lincomycins, Liothyronines, Luteinizing hormones, Lyme disease vaccines, Mangafodipirs, Manthtols, Meningococcal polysaccharide vaccines, Meperidines, Mepivacaines, Mesoridazines, Metaraminols, Methadones, Methocarbamols, Methohexitals, Methyldopates, Methylergonovines, Metoclopramides, Metoprolols, Metronidazoles, Minocyclines, Mivacuriums, Morrhuic acids, Moxifloxacins, Muromonab-CD3s, Mycophenolate mofetils, Nafcillins, Nalbuphines, Nalmefenes, Naloxones, Neostigmines, Niacinamides, Nicardipines, Nitroglycerins, Nitroprussides, Norepinephrines, Orphenadrines, Oxacillins, Oxymorphones, Oxytetracyclines, Oxytocins, Pancuroniums, Panthenols, Pantothenic acids, Papaverines, Peginterferon alpha 2As, Penicillin Gs, Pentamidines, Pentazocines, Pentobarbitals, Perflutrens, Perphenazines, Phenobarbitals, Phentolamines, Phenylephrines, Phenytoins, Physostigmines, Phytonadiones, Polymyxin, Pralidoximes, Prilocaines, Procainamides, Procaines, Prochlorperazines, Progesterones, Propranolols, Pyridostigmine hydroxides, Pyridoxines, Quinidines, Quinupristins, Rabies immunoglobulins, Rabies vaccines, Ranitidines, Remifentanils, Riboflavins, Rifampins, Ropivacaines, Samariums, Scopolamines, Seleniums, Sermorelins, Sincalides, Somatrems, Spectinomycins, Streptokinases, Streptomycins, Succinylcholines, Sufentanils, Sulfamethoxazoles, Tacrolimuses, Terbutalines, Teriparatides, Testosterones, Tetanus antitoxins, Tetracaines, Tetradecyl sulfates, Theophyllines, Thiamines, Thiethylperazines, Thiopentals, Thyroid stimulating hormones, Tinzaparins, Tirofibans, Tobramycins, Tolazolines, Tolbutamides, Torsemides, Tranexamic acids, Treprostinils, Trifluoperazines, Trimethobenzamides, Trimethoprims, Tromethamines, Tuberculins, Typhoid vaccines, Urofollitropins, Urokinases, Valproic acids, Vasopressins, Vecuroniums, Verapamils, Voriconazoles, Warfarins, Yellow fever vaccines, Zidovudines, Zincs, Ziprasidone hydrochlorides, Aclacinomycins, Actinomycins, Adriamycins, Azaserines, 6-Azauridines, Carzinophilins, Chromomycins, Denopterins, 6 Diazo 5 Oxo-L-Norleucines, Enocitabines, Floxuridines, Olivomycins, Pirarubicins, Piritrexims, Pteropterins, Tegafurs, Tubercidins, Alteplases, Arcitumomabs, bevacizumabs, Botulinum Toxin Type As, Botulinum Toxin Type Bs, Capromab Pendetides, Daclizumabs, Dornase alphas, Drotrecogin alphas, Imciromab Pentetates, Iodine-131s, an antibiotic agent, an angiogenesis inhibitor, anti-cataract and anti-diabetic retinopathy substances, carbonic anhydrase inhibitors, mydriatics, photodynamic therapy agents, prostaglandin analogs, growth factor, anti-neoplastics, anti-metabolites, anti-viral, amebicides, anti-protozoals, anti-tuberculosis agents, anti-leprotics, antitoxins and antivenins, antihemophilic factor, anti-inhibitor coagulant complex, antithrombin III, coagulations Factor V, coagulation Factor IX, plasma protein fraction, von Willebrand factor, an antiplatelet agent, a colony stimulating factor (CSF), an erythropoiesis stimulator, hemostatics, albumins, Immune Globulins, thrombin inhibitors, anticoagulants, a steroidal anti-inflammatory drug selected from among alclometasones, algestones, beclomethasones, betamethasones, budesonides, clobetasols, clobetasones, clocortolones, cloprednols, corticosterones, cortisones, cortivazols, deflazacorts, desonides, desoximetasones, dexamethasones, diflorasones, diflucortolones, difluprednates, enoxolones, fluazacorts, flucloronides, flumethasones, flunisolides, fluocinolones, fluocinonides, fluocortins, fluocortolones, fluorometholones, fluperolones, fluprednidenes, fluprednisolones, flurandrenolides, fluticasones, formocortals, halcinonides, halobetasols, halometasones, halopredones, hydrocortamates, hydrocortisones, loteprednol etabonate, mazipredones, medrysones, meprednisones, methylprednisolones, mometasone furoate, paramethasones, prednicarbates, prednisolones, prednisones, prednivals, prednylidenes, rimexolones, tixocortols and triamcinolones, Docosenoid, prostaglandins, prostaglandin analogs, antiprostaglandins, prostaglandin precursors, miotics, cholinergics, anti-cholinesterase, or anti-allergenics.

Provided herein is a system for the non-refrigerated storage of a stable PH20 hyaluronidase formulation that contains any of the modified PH20 polypeptides provided herein or any of the pharmaceutical compositions provided herein and a container suitable for storage without refrigeration. Typically the modified PH20 polypeptide or the pharmaceutical composition containing the modified PH20 polypeptide is provided as a liquid. The container can be a vial, syringe, tube or bag or other container. The container can be glass or plastic.

Provided herein is a method of preparing a pharmaceutical composition containing a PH20 hyaluronidase that can be stored for direct administration without refrigeration that includes providing any of the modified PH20 polypeptides provided herein, and formulating the polypeptide as a liquid with a pharmaceutically acceptable buffering agent for parenteral administration, such as for intravenous or subcutaneous administration. In examples of the method, the amount of buffering agent is an amount sufficient to maintain a pH range of between or about between 6.0 to 7.8, inclusive, for example, a pH range of between or about between 6.5 to 7.5, inclusive. The buffering agent can be Tris, histidine, phosphate or citrate, such as sodium phosphate. In examples of the above method, the amount of buffering agent is between 1 mM to 100 mM. In any of the above examples of the method, the PH20 polypeptide is formulated in the absence of a stabilizer that is an amino acid, an amino acid derivative, an amine, a sugar, a polyol, a surfactant, a preservative, a hyaluronidase inhibitor or an albumin protein. In other examples of the above method, the PH20 polypeptide is formulated in the absence of salt or is formulated with a concentration of salt that is less than 130 mM. Also provided herein is a pharmaceutical composition that is prepared by any of the above methods.

Provided herein is a method for treating a hyaluronan-associated disease or condition, by administering to a subject any of the pharmaceutical compositions provided herein. The hyaluronan-associated disease or condition is an inflammatory disease or a tumor or cancer. For example, the hyaluronan-associated disease or condition is an edema, cardiovascular disease, tumor or cancer or other disease or condition as described herein caused by or associated with accumulated or excess hyaluronan. For example, the hyaluronan-associated disease or condition is a tumor or cancer, such as one where the tumor is a solid tumor. The hyaluronan-associated disease or condition can be late-stage cancers, metastatic cancers or an undifferentiated cancers. In particular examples, the hyaluronan-associated disease or condition is an ovarian cancer, in situ carcinoma (ISC), squamous cell carcinoma (SCC), prostate cancer, pancreatic cancer, non-small cell lung cancer, breast cancer or colon cancer.

Also provided herein is a method for increasing delivery of a therapeutic agent to a subject, by administering a subject any of the pharmaceutical compositions and a therapeutic agent. In some examples of the method herein, any of the combinations provided herein containing a therapeutic agent is administered to the subject. In examples of such methods, the administration is subcutaneous. The composition containing a modified PH20 polypeptide can be administered prior to, simultaneously, intermittently or subsequent to administration of the therapeutic agent.

In any of the examples of the above method for increasing delivery of a therapeutic agent, the therapeutic agent is a polypeptide, a protein, a nucleic acid, a drug, a small molecule or an organic molecule. For example, the therapeutic agent is a chemotherapeutic agent, an analgesic agent, an anti-inflammatory agent, an antimicrobial agent, an amoebicidal agent, a trichomonocidal agent, an anti-Parkinson agent, an anti-malarial agent, an anticonvulsant agent, an anti-depressant agent, and antiarthritics agent, an anti-fungal agent, an antihypertensive agent, an antipyretic agent, an anti-parasite agent, an antihistamine agent, an alpha-adrenargic agonist agent, an alpha blocker agent, an anesthetic agent, a bronchial dilator agent, a biocide agent, a bactericide agent, a bacteriostat agent, a beta adrenergic blocker agent, a calcium channel blocker agent, a cardiovascular drug agent, a contraceptive agent, a decongestant agent, a diuretic agent, a depressant agent, a diagnostic agent, an electrolyte agent, a hypnotic agent, a hormone agent, a hyperglycemic agent, a muscle relaxant agent, a muscle contractant agent, an ophthalmic agent, a parasympathomimetic agent, a psychic energizer agent, a sedative agent, a sympathomimetic agent, a tranquilizer agent, a urinary agent, a vaginal agent, a viricide agent, a vitamin agent, a non-steroidal anti-inflammatory agent, an angiotensin converting enzyme inhibitor agent, or a sleep inducer. In particular examples, the therapeutic agent is selected from among an antibody, an Immune Globulin, a bisphosphonate, a cytokine, a chemotherapeutic agent, a coagulation factor and an insulin, such as a fast-acting insulin. In other examples, the therapeutic agent is selected from among Adalimumabs, Agalsidase Betas, Alefacepts, Ampicillins, Anakinras, Antipoliomyelitic Vaccines, Anti-Thymocytes, Azithromycins, Becaplermins, Caspofungins, Cefazolins, Cefepimes, Cefotetans, Ceftazidimes, Ceftriaxones, Cetuximabs, Cilastatins, Clavulanic Acids, Clindamycins, Darbepoetin Alfas, Daclizumabs, Diphtheria, Diphtheria antitoxins, Diphtheria Toxoids, Efalizumabs, Epinephrines, Erythropoietin Alphas, Etanercepts, Filgrastims, Fluconazoles, Follicle-Stimulating Hormones, Follitropin Alphas, Follitropin Betas, Fosphenytoins, Gadodiamides, Gadopentetates, Gatifloxacins, Glatiramers, Granulocyte macrophage colony-stimulating factors (GM-CSFs), Goserelin acetates, Granisetrons, Haemophilus Influenza Bs, Haloperidols, Hepatitis vaccines, Hepatitis A Vaccines, Hepatitis B Vaccines, Ibritumomab Tiuxetans, Ibritumomabs, Tiuxetans, Immunoglobulins, Hemophilus influenza vaccines, Influenza Virus Vaccines, Infliximabs, Insulin lispro, 75% neutral protamine lispro (NPL)/25% insulin lispro, 50% neutral protamine Hagedorn (NPH)/50% regular insulin, 70% NPH/30% regular insulin, Regular insulin, NPH insulin, Ultra insulin, Ultralente insulin, Insulin Glargines, Interferons, Interferon alphas, Interferon betas, Interferon gammas, Interferon alpha-2a, Interferon alpha-2b, Interferon Alphacon, Interferon alpha-n, Interferon Betas, Interferon Beta-1as, Interferon Gammas, Interferon alpha-con, Iodixanols, Iohexols, Iopamidols, Ioversols, Ketorolacs, Laronidases, Levofloxacins, Lidocaines, Linezolids, Lorazepams, Measles Vaccines, Measles virus, Mumps viruses, Measles-Mumps-Rubella Virus Vaccines, Rubella vaccines, Medroxyprogesterones, Meropenems, Methylprednisolones, Midazolams, Morphines, Octreotides, Omalizumabs, Ondansetrons, Palivizumabs, Pantoprazoles, Pegaspargases, Pegfilgrastims, Peg-Interferon Alpha-2as, Peg-Interferon Alpha-2bs, Pegvisomants, Pertussis vaccines, Piperacillins, Pneumococcal Vaccines Pneumococcal Conjugate Vaccines, Promethazines, Reteplases, Somatropins, Sulbactams, Sumatriptans, Tazobactams, Tenecteplases, Tetanus Purified Toxoids, Ticarcillins, Tositumomabs, Triamcinolones, Triamcinolone Acetonides, Triamcinolone hexacetonides, Vancomycins, Varicella Zoster immunoglobulins, Varicella vaccines, other vaccines, Alemtuzumabs, Alitretinoins, Allopurinols, Altretamines, Amifostines, Anastrozoles, Arsenics, Arsenic Trioxides, Asparaginases, Bacillus Calmette-Guerin (BCG) vaccines, BCG Live, Bexarotenes, Bleomycins, Busulfans, Busulfan intravenous, Busulfan orals, Calusterones, Capecitabines, Carboplatins, Carmustines, Carmustines with Polifeprosans, Celecoxibs, Chlorambucils, Cisplatins, Cladribines, Cyclophosphamides, Cytarabines, Cytarabine liposomals, Dacarbazines, Dactinomycins, Daunorubicin liposomals, Daunorubicins, Denileukin Diftitoxes, Dexrazoxanes, Docetaxels, Doxorubicins, Doxorubicin liposomals, Dromostanolone propionates, Elliotts B Solutions, Epirubicins, Epoetin alfas, Estramustines, Etoposide phosphates, Exemestanes, Floxuridines, Fludarabines, Fluorouracils, Fulvestrants, Gemcitabines, Gemtuzumabs, Ozogamicins, Gemtuzumab ozogamicins, Hydroxyureas, Idarubicins, Ifosfamides, Imatinib mesylates, Irinotecans, Letrozoles, Leucovorins, Levamisoles, Lomustines, Mechlorethamines, Nitrogen mustards, Megestrols, Megestrol acetates, Melphalans, Mercaptopurines, Mesnas, Methotrexates, Methoxsalens, Mitomycins, Mitomycin Cs, Mitotanes, Mitoxantrones, Nandrolones, Nandrolone Phenpropionates, Nofetumomabs, Oprelvekins, Oxaliplatins, Paclitaxels, Pamidronates, Pegademases, Pentostatins, Pipobromans, Plicamycins, Porfimer sodiums, Procarbazines, Quinacrines, Rasburicases, Rituximabs, Sargramostims, Streptozocins, Talcs, Tamoxifens, Temozolomides, Teniposides, Testolactones, Thioguanines, Triethylenethiophosphoramides (Thiotepas), Topotecans, Toremifenes, Trastuzumabs, Tretinoins, Uracil Mustards, Valrubicins, Vinblastines, Vincristines, Vinorelbines, Zoledronates, Acivicins, Aclarubicins, Acodazoles, Acronines, Adozelesins, Retinoic Acids, 9-Cis-Retinoic Acids, Alvocidibs, Ambazones, Ambomycins, Ametantrones, Aminoglutethimides, Amsacrines, Anaxirones, Ancitabines, Anthramycins, Apaziquones, Argimesnas, Asperlins, Atrimustines, Azacitidines, Azetepas, Azotomycins, Banoxantrones, Batabulins, Batimastats, Benaxibines, Bendamustines, Benzodepas, Bicalutamides, Bietaserpines, Biricodars, Bisantrenes, Bisnafide Dimesylates, Bizelesins, Bortezomibs, Brequinars, Bropirimines, Budotitanes, Cactinomycins, Canertinibs, Caracemides, Carbetimers, Carboquones, Carmofurs, Carubicins, Carzelesins, Cedefingols, Cemadotins, Cioteronels, Cirolemycins, Clanfenurs, Clofarabines, Crisnatols, Decitabines, Dexniguldipines, Dexormaplatins, Dezaguanines, Diaziquones, Dibrospidiums, Dienogests, Dinalins, Disermolides, Dofequidars, Doxifluridines, Droloxifenes, Duazomycins, Ecomustines, Edatrexates, Edotecarins, Eflomithines, Elacridars, Elinafides, Elsamitrucins, Emitefurs, Enloplatins, Enpromates, Enzastaurins, Epipropidines, Eptaloprosts, Erbulozoles, Esorubicins, Etanidazoles, Etoglucids, Etoprines, Exisulinds, Fadrozoles, Fazarabines, Fenretinides, Fluoxymesterones, Flurocitabines, Fosquidones, Fostriecins, Fotretamines, Galarubicins, Galocitabines, Geroquinols, Gimatecans, Gimeracils, Gloxazones, Glufosfamides, Ilmofosines, Ilomastats, Imexons, Improsulfans, Indisulams, Inproquones, Interleukins, Interleukin-2s, recombinant Interleukins, Intoplicines, Iobenguanes, Iproplatins, Irsogladines, Ixabepilones, Ketotrexates, L-Alanosines, Lanreotides, Lapatinibs, Ledoxantrones, Leuprolides, Lexacalcitols, Liarozoles, Lobaplatins, Lometrexols, Lonafarnibs, Losoxantrones, Lurtotecans, Mafosfamides, Mannosulfans, Marimastats, Masoprocols, Maytansines, Melengestrols, Menogarils, Mepitiostanes, Metesinds, Metomidates, Metoprines, Meturedepas, Miboplatins, Miproxifenes, Misonidazoles, Mitindomides, Mitocarcins, Mitocromins, Mitoflaxones, Mitogillins, Mitoguazones, Mitomalcins, Mitonafides, Mitoquidones, Mitospers, Mitozolomides, Mivobulins, Mizoribines, Mofarotenes, Mopidamols, Mubritinibs, Mycophenolic Acids, Nedaplatins, Nelarabines, Nemorubicins, Nitracrines, Nocodazoles, Nogalamycins, Nolatrexeds, Nortopixantrones, Ormaplatins, Ortataxels, Oteracils, Oxisurans, Oxophenarsines, Patupilones, Peldesines, Peliomycins, Pelitrexols, Pemetrexeds, Pentamustines, Peplomycins, Perfosfamides, Perifosines, Picoplatins, Pinafides, Piposulfans, Pirfenidones, Piroxantrones, Pixantrones, Plevitrexeds, Plomestanes, Porfiromycins, Prednimustines, Propamidines, Prospidiums, Pumitepas, Puromycins, Pyrazofurins, Ranimustines, Riboprines, Ritrosulfans, Rogletimides, Roquinimexs, Sabarubicins, Safingols, Satraplatins, Sebriplatins, Semustines, Simtrazenes, Sizofirans, Sobuzoxanes, Sorafenibs, Sparfosates, Sparfosic Acids, Sparsomycins, Spirogermaniums, Spiromustines, Spiroplatins, Squalamines, Streptonigrins, Streptovarycins, Sufosfamides, Sulofenurs, Tacedinalines, Talisomycins, Tallimustines, Tariquidars, Tauromustines, Tecogalans, Tegafurs, Teloxantrones, Temoporfins, Teroxirones, Thiamiprines, Tiamiprines, Tiazofurins, Tilomisoles, Tilorones, Timcodars, Timonacics, Tirapazamines, Topixantrones, Trabectedins, Trestolones, Triciribines, Trilostanes, Trimetrexates, Triplatin Tetranitrates, Triptorelins, Trofosfamides, Tubulozoles, Ubenimexs, Uredepas, Valspodars, Vapreotides, Verteporfins, Vindesines, Vinepidines, Vinflunines, Vinformides, Vinglycinates, Vinleucinols, Vinleurosines, Vinrosidines, Vintriptols, Vinzolidines, Vorozoles, Xanthomycin As, Guamecyclines, Zeniplatins, Zilascorbs [2-H], Zinostatins, Zorubicins, Zosuquidars, Acetazolamides, Acyclovirs, Adipiodones, Alatrofloxacins, Alfentanils, Allergenic extracts, Alpha 1-proteinase inhibitors, Alprostadils, Amikacins, Amino acids, Aminocaproic acids, Aminophyllines, Amitriptylines, Amobarbitals, Amrinones, Analgesics, Anti-poliomyelitis vaccines, Anti-rabic serums, Anti-tetanus immunoglobulins, tetanus vaccines, Antithrombin IIIs, Antivenom serums, Argatrobans, Arginines, Ascorbic acids, Atenolols, Atracuriums, Atropines, Aurothioglucoses, Azathioprines, Aztreonams, Bacitracins, Baclofens, Basiliximabs, Benzoic acids, Benztropines, Betamethasones, Biotins, Bivalirudins, Botulism antitoxins, Bretyliums, Bumetanides, Bupivacaines, Buprenorphines, Butorphanols, Calcitonins, Calcitriols, Calciums, Capreomycins, Carboprosts, Carnitines, Cefamandoles, Cefoperazones, Cefotaximes, Cefoxitins, Ceftizoximes, Cefuroximes, Chloramphenicols, Chloroprocaines, Chloroquines, Chlorothiazides, Chlorpromazines, Chondroitinsulfuric acids, Choriogonadotropin alfas, Chromiums, Cidofovirs, Cimetidines, Ciprofloxacins, Cisatracuriums, Clonidines, Codeines, Colchicines, Colistins, Collagens, Corticorelin ovine triflutates, Corticotrophins, Cosyntropins, Cyanocobalamins, Cyclosporines, Cysteines, Dacliximabs, Dalfopristins, Dalteparins, Danaparoids, Dantrolenes, Deferoxamines, Desmopressins, Dexamethasones, Dexmedetomidines, Dexpanthenols, Dextrans, Iron dextrans, Diatrizoic acids, Diazepams, Diazoxides, Dicyclomines, Digibinds, Digoxins, Dihydroergotamines, Diltiazems, Diphenhydramines, Dipyridamoles, Dobutamines, Dopamines, Doxacuriums, Doxaprams, Doxercalciferols, Doxycyclines, Droperidols, Dyphyllines, Edetic acids, Edrophoniums, Enalaprilats, Ephedrines, Epoprostenols, Ergocalciferols, Ergonovines, Ertapenems, Erythromycins, Esmolols, Estradiols, Estrogenics, Ethacrynic acids, Ethanolamines, Ethanols, Ethiodized oils, Etidronic acids, Etomidates, Famotidines, Fenoldopams, Fentanyls, Flumazenils, Fluoresceins, Fluphenazines, Folic acids, Fomepizoles, Fomivirsens, Fondaparinuxs, Foscarnets, Fosphenytoins, Furosemides, Gadoteridols, Gadoversetamides, Ganciclovirs, Gentamicins, Glucagons, Glucoses, Glycines, Glycopyrrolates, Gonadorelins, Gonadotropin chorionics, Haemophilus B polysaccharides, Hemins, Herbals, Histamines, Hydralazines, Hydrocortisones, Hydromorphones, Hydroxocobalamins, Hydroxyzines, Hyoscyamines, Ibutilides, Imiglucerases, Indigo carmines, Indomethacins, Iodides, Iopromides, Iothalamic acids, Ioxaglic acids, Ioxilans, Isoniazids, Isoproterenols, Japanese encephalitis vaccines, Kanamycins, Ketamines, Labetalols, Lepirudins, Levobupivacaines, Levothyroxines, Lincomycins, Liothyronines, Luteinizing hormones, Lyme disease vaccines, Mangafodipirs, Manthtols, Meningococcal polysaccharide vaccines, Meperidines, Mepivacaines, Mesoridazines, Metaraminols, Methadones, Methocarbamols, Methohexitals, Methyldopates, Methylergonovines, Metoclopramides, Metoprolols, Metronidazoles, Minocyclines, Mivacuriums, Morrhuic acids, Moxifloxacins, Muromonab-CD3s, Mycophenolate mofetils, Nafcillins, Nalbuphines, Nalmefenes, Naloxones, Neostigmines, Niacinamides, Nicardipines, Nitroglycerins, Nitroprussides, Norepinephrines, Orphenadrines, Oxacillins, Oxymorphones, Oxytetracyclines, Oxytocins, Pancuroniums, Panthenols, Pantothenic acids, Papaverines, Peginterferon alpha 2As, Penicillin Gs, Pentamidines, Pentazocines, Pentobarbitals, Perflutrens, Perphenazines, Phenobarbitals, Phentolamines, Phenylephrines, Phenytoins, Physostigmines, Phytonadiones, Polymyxin, Pralidoximes, Prilocaines, Procainamides, Procaines, Prochlorperazines, Progesterones, Propranolols, Pyridostigmine hydroxides, Pyridoxines, Quinidines, Quinupristins, Rabies immunoglobulins, Rabies vaccines, Ranitidines, Remifentanils, Riboflavins, Rifampins, Ropivacaines, Samariums, Scopolamines, Seleniums, Sermorelins, Sincalides, Somatrems, Spectinomycins, Streptokinases, Streptomycins, Succinylcholines, Sufentanils, Sulfamethoxazoles, Tacrolimuses, Terbutalines, Teriparatides, Testosterones, Tetanus antitoxins, Tetracaines, Tetradecyl sulfates, Theophyllines, Thiamines, Thiethylperazines, Thiopentals, Thyroid stimulating hormones, Tinzaparins, Tirofibans, Tobramycins, Tolazolines, Tolbutamides, Torsemides, Tranexamic acids, Treprostinils, Trifluoperazines, Trimethobenzamides, Trimethoprims, Tromethamines, Tuberculins, Typhoid vaccines, Urofollitropins, Urokinases, Valproic acids, Vasopressins, Vecuroniums, Verapamils, Voriconazoles, Warfarins, Yellow fever vaccines, Zidovudines, Zincs, Ziprasidone hydrochlorides, Aclacinomycins, Actinomycins, Adriamycins, Azaserines, 6-Azauridines, Carzinophilins, Chromomycins, Denopterins, 6 Diazo 5 Oxo-L-Norleucines, Enocitabines, Floxuridines, Olivomycins, Pirarubicins, Piritrexims, Pteropterins, Tegafurs, Tubercidins, Alteplases, Arcitumomabs, bevacizumabs, Botulinum Toxin Type As, Botulinum Toxin Type Bs, Capromab Pendetides, Daclizumabs, Dornase alphas, Drotrecogin alphas, Imciromab Pentetates, Iodine-131s, an antibiotic agent, an angiogenesis inhibitor, anti-cataract and anti-diabetic retinopathy substances, carbonic anhydrase inhibitors, mydriatics, photodynamic therapy agents, prostaglandin analogs, growth factor, anti-neoplastics, anti-metabolites, anti-viral, amebicides, anti-protozoals, anti-tuberculosis agents, anti-leprotics, antitoxins and antivenins, antihemophilic factor, anti-inhibitor coagulant complex, antithrombin III, coagulations Factor V, coagulation Factor IX, plasma protein fraction, von Willebrand factor, an antiplatelet agent, a colony stimulating factor (CSF), an erythropoiesis stimulator, hemostatics, albumins, Immune Globulins, thrombin inhibitors, anticoagulants, a steroidal anti-inflammatory drug selected from among alclometasones, algestones, beclomethasones, betamethasones, budesonides, clobetasols, clobetasones, clocortolones, cloprednols, corticosterones, cortisones, cortivazols, deflazacorts, desonides, desoximetasones, dexamethasones, diflorasones, diflucortolones, difluprednates, enoxolones, fluazacorts, flucloronides, flumethasones, flunisolides, fluocinolones, fluocinonides, fluocortins, fluocortolones, fluorometholones, fluperolones, fluprednidenes, fluprednisolones, flurandrenolides, fluticasones, formocortals, halcinonides, halobetasols, halometasones, halopredones, hydrocortamates, hydrocortisones, loteprednol etabonate, mazipredones, medrysones, meprednisones, methylprednisolones, mometasone furoate, paramethasones, prednicarbates, prednisolones, prednisones, prednivals, prednylidenes, rimexolones, tixocortols and triamcinolones, Docosenoid, prostaglandins, prostaglandin analogs, antiprostaglandins, prostaglandin precursors, miotics, cholinergics, anti-cholinesterase, or anti-allergenics.

In any of the above method of treating a subject, the composition that is administered is one that has been or is stored without refrigeration prior to administration to the subject. In any of the methods herein, the method can include storing the composition without refrigeration prior to administration to the subject. In such examples, storing the composition without refrigeration exposes the composition to an ambient temperature that is between 18° C. to 45° C., 25° C. to 42° C. or 30° C. to 37° C., for example, to an ambient temperature greater than 25° C. The storage of the composition without refrigeration can be for greater than 48 hours, 72 hours, 96 hours, one week, two weeks, three weeks, one month, two months, three months, four months, five months or six months.

Also provided herein are medical uses of any of the pharmaceutical compositions or combinations provided herein for treating a hyaluronan-disease or disorder or for increasing the delivery of a therapeutic agent for treating a disease or condition treatable by the therapeutic agent. For example, provided herein are any of the pharmaceutical compositions provided herein or combinations provided herein for use in treating a hyaluronan-associated disease or disorder, such as an edema, cardiovascular disease, tumor or cancer or other hyaluronan-associated disease or disorder described herein or known to a skilled artisan. Also provided herein are any of the pharmaceutical compositions provided herein or combinations provided herein for use in delivering a therapeutic agent to a subject. The therapeutic agent can be any therapeutic agent that is known to treat a disease or condition, such as any described herein above or elsewhere herein. In any of the above examples of medical uses, including pharmaceutical compositions or combinations for use, the composition containing a modified PH20 is a non-refrigerated composition. Hence, provided herein are medical uses of a non-refrigerated PH20 pharmaceutical composition for treating a hyaluronan-associated disease or condition. Also provided herein are medical uses of a non-refrigerated PH20 for use in increasing delivery of a therapeutic agent, for example, for treating a disease or condition that is treated or treatable by the therapeutic agent.

Provided herein is a method for identifying or selecting a modified hyaluronan-degrading enzyme that exhibits thermal stability that contains the steps of a) testing the activity of a modified hyaluronan-degrading enzyme or a member of a collection of modified hyaluronan-degrading enzymes after incubation at a temperature for a predetermined time that provides a thermal stress condition to the unmodified hyaluronan-degrading enzyme not containing a modification; b) testing the activity of the modified hyaluronan-degrading enzyme or a member of a collection of modified hyaluronan-degrading enzymes after incubation at 2° C. to 8° C., wherein in the activity is tested under the same conditions as a) except for the difference in temperature; and c) selecting or identifying a modified hyaluronan-degrading enzyme that exhibits activity in a) that is at least 50% of the activity in b). In aspects of the method, in step c) a modified hyaluronan-degrading enzyme is selected or identified if the activity in a) is at least 55%, 60%, 65%, 70%, 75%, 80%, 85%, 90%, 95% or more of the activity in b). In any of the examples of a method of identifying or selecting a modified hyaluronan-degrading enzyme, the activity is hyaluronidase activity.

In examples of the above method of identifying or selecting a modified hyaluronan-degrading enzyme, the method can further include the steps of d) comparing the activity of the selected or identified modified hyaluronan-degrading enzyme in b) to the activity of the unmodified hyaluronan-degrading enzyme tested under the same conditions; and e) identifying or selecting a modified hyaluronan-degrading enzyme that exhibits at least 40%, 50%, 60%, 70%, 80%, 90%, 100% or more of the activity compared to the unmodified hyaluronan-degrading enzyme.

In any of the examples of a method of a method of identifying or selecting a modified hyalurnan-degrading enzyme, the thermal stress condition is a temperature that is or is greater than the T₅₀ of the unmodified hyaluronan-degrading enzyme not containing a modification as determined in a thermal challenge assay at the predetermined time. For example, the activity in a) is tested at a temperature that is at least 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., 20° C. or more greater than the T₅₀ of the unmodified hyaluronan-degrading enzyme as determined in a thermal challenge assay at the predetermined time. In aspects of the method, prior to step a), the method can include a step of determining the T₅₀ of the unmodified hyaluronan-degrading enzyme as determined in a thermal challenge assay at the predetermined time.

In other examples of any of the methods of identifying or selecting a modified hyaluronan-degrading enzyme provided, herein, the thermal stress condition is a temperature that is or is greater than the melting temperature (Tm) of the unmodified hyaluronan-degrading enzyme not containing a modification. For example, the activity in a) is tested at a temperature that is at least 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., 20° C. or more greater than the melting temperature (Tm) of the hyaluronan-degrading enzyme. In aspects of the method, prior to step a), the method can include a step of determining the melting temperature (Tm) of the hyaluronan-degrading enzyme. For example, the melting temperature (Tm) can be determined by dynamic light scattering, circular dichroism (CD) spectroscopy, fluorescence emission spectroscopy or nuclear magnetic resonance (NMR) spectroscopy.

In any of the examples of a method of identifying or selecting a modified hyaluronan-degrading enzyme, the activity in a) is tested at a temperature that is greater than 44° C., for example, greater than 45° C., 46° C., 47° C., 48° C., 49° C., 50° C., 51° C., 52° C., 53° C., 54° C., 55° C., 56° C., 57° C., 58° C., 59° C., 60° C. or greater. In particular examples of the method herein, the activity in a) is tested at a temperature that is greater than or is or is about 52° C. In these and any other examples of the methods herein, the hyaluronan-degrading enzyme, such as a modified hyaluronan-degrading enzyme, is incubated in step a) and step b) for a predetermined time that is at least 5 minutes, 10 minutes, 20 minutes, 30 minutes, 1 hour, 2 hours, 3 hours, 4 hours, 5 hours, 6 hours or more. For example, in examples of the method herein, the thermal stress condition in a) is incubation at a temperature that is greater than or is 52° C. for 10 minutes. Therefore, the condition in b) is incubation at a temperature that is 2° C. to 8° C., such as or about 4° C., for 10 minutes.

In any of the examples of a method of identifying or selecting a modified hyaluronan-degrading enzyme, the modified hyaluronan-degrading enzyme contains an amino acid replacement, insertion or deletion of amino acids compared to an unmodified hyaluronan-degrading enzyme. In particular examples, the modified hyaluronan-degrading enzyme contains an amino acid replacement or amino acid replacements. For example, the modified hyaluronan-degrading enzyme contains a single amino acid replacement or two, three, four, five, six, seven, eight, nine or more amino acid replacements compared to an unmodified form of the hyaluronan-degrading enzyme.

In any of the examples of a method of identifying or selecting a modified hyaluronan-degrading enzyme, a member of a collection of modified hyaluronan-degrading enzyme are tested in a) and/or b); and a plurality of modified hyaluronan-degrading enzymes are separately tested in a) and/or b). In such examples, the plurality of modified hyaluronan-degrading enzymes are modified compared to the corresponding unmodified hyaluronan-degrading enzyme to generate a collection of modified hyaluronan-degrading enzymes, whereby each modified protein in the collection is tested in each of a) and/or b), wherein each modified hyaluronan-degrading enzyme in the collection contains a single amino acid replacement compared to the unmodified form of the hyaluronan-degrading enzyme. For example, in the collection, the amino acid at each modified position is replaced by up to 1-19 other amino acids other than the original amino acid at the position, whereby each modified hyaluronan-degrading enzyme contains a different amino acid replacement. In particular examples of the generated collection, every amino acid along the length of the hyaluronan-degrading enzyme, or a selected portion thereof, is replaced.

In any of the examples of the method of identifying or selecting a modified hyaluronan-degrading enzyme, the hyaluronan-degrading enzyme that is tested is modified, for example by amino acid replacement or replacements, compared to an unmodified hyaluronan-degrading enzyme. The unmodified hyaluronan-degrading enzyme can be a chondroitinase or a hyaluronidase. For example, the unmodified hyaluronan-degrading enzyme is a hyaluronidase that is a PH20 hyaluronidase or truncated form thereof lacking a C-terminal glycosylphosphatidylinositol (GPI) anchor attachment site or a portion of the GPI anchor attachment site, whereby the truncated form exhibits hyaluronidase activity. The PH20 can be a human, monkey, bovine, ovine, rat, fox, mouse or guinea pig PH20. For example, the unmodified hyaluronan-degrading enzyme has the sequence of amino acids set forth in any of SEQ ID NOS: 3, 7, 10, 12, 14, 24, 32-66, 69, 72, 388, 390, 392, or 400 or a sequence of amino acids that is at least 80% sequence identity to any of SEQ ID NOS: 3, 7, 10, 12, 14, 24, 32-66, 69, 72, 388, 390, 392, or 400, such as at least 85%, 86%, 87%, 88%, 89%, 90%, 91%, 92%, 93%, 94%, 95%, 96%, 97%, 98%, 99% sequence identity to any of SEQ ID NOS: 3, 7, 10, 12, 14, 24, 32-66, 69, 72, 388, 390, 392, or 400. In particular, the PH20 is a human PH20 or a C-terminal truncated form thereof that is soluble. For example, the unmodified hyaluronan-degrading enzyme is a PH20 hyaluronidase having the sequence of amino acids set forth in any of SEQ ID NOS: 3, 7, 32-66, 69 or 72, or a sequence of amino acids that exhibits at least 85% sequence identity to any of SEQ ID NOS: 3, 7, 32-66, 69 or 72, such as at least 86%, 87%, 88%, 89%, 90%, 91%, 92%, 93%, 94%, 95%, 96%, 97%, 98%, 99% or more sequence identity to any of SEQ ID NOS: 3, 7, 32-66, 69 or 72.

In any of the examples of a method herein of identifying or selecting a modified hyaluronan-degrading enzyme that exhibits thermal stability, the method is performed in vitro. The method also can be performed by repeating any of the above steps a plurality of times, wherein in each repetition, further modified hyaluronan-degrading enzymes of a selected modified hyaluronan-degrading enzyme are generated and tested, whereby the modified hyaluronan-degrading enzyme is evolved to exhibit increased stability under a denaturation condition.

Also provided herein is a modified hyaluronan-degrading enzyme identified or selected by any of the above methods of identifying or selecting a modified hyaluronan-degrading enzyme that exhibits thermal stability.

### BRIEF DESCRIPTION OF THE FIGURES

FIG. 1 depicts the amino acid sequence of full-length human PH20 (set forth in SEQ ID NO:7) and soluble C-terminal truncated variants thereof. The C-terminal amino acid residue of exemplary C-terminal truncated variants of full-length PH20 are indicated by bold font. The complete amino acid sequences of exemplary C-terminal truncated variants of full-length PH20 also are provided in SEQ ID NOS: 3 and 32-66. The C-terminal amino acid residue of an exemplary soluble PH20, whose complete sequence is set forth in SEQ ID NO:3, also is indicated by underline. Exemplary, non-limiting, positions for amino acid replacements are indicated by highlighting. Corresponding positions of these and other amino acid replacements described herein can be identified by alignment of a sequence of interest with any of SEQ ID NOS: 3, 7 or 32-66, and in particular with SEQ ID NO:3.

FIG. 2 (A-L) depicts exemplary alignments of human soluble PH20 set forth in SEQ ID NO:3 with other PH20 polypeptides. A “*” means that the aligned residues are identical, a “:” means that aligned residues are not identical, but are similar and contain conservative amino acids residues at the aligned position, and a “.” means that the aligned residues are similar and contain semi-conservative amino acid residues at the aligned position. Exemplary, non-limiting, corresponding positions for amino acid replacements are indicated by highlighting. For example, FIG. 2A depicts the alignment of a human soluble PH20 set forth in SEQ ID NO:3 with chimpanzee PH20 set forth in SEQ ID NO:10. FIG. 2B depicts the alignment of a human soluble PH20 set forth in SEQ ID NO:3 with Rhesus monkey PH20 set forth in SEQ ID NO:12. FIG. 2C depicts the alignment of a human soluble PH20 set forth in SEQ ID NO:3 with Cynomolgus monkey PH20 set forth in SEQ ID NO:14. FIG. 2D depicts the alignment of human soluble PH20 set forth in SEQ ID NO:3 with bovine PH20 set forth in SEQ ID NO:16. FIG. 2E depicts the alignment of a human soluble PH20 set forth in SEQ ID NO:3 with mouse PH20 set forth in SEQ ID NO:20. FIG. 2F depicts the alignment of a human soluble PH20 set forth in SEQ ID NO:3 with rat PH20 set forth in SEQ ID NO:22. FIG. 2G depicts the alignment of a human soluble PH20 set forth in SEQ ID NO:3 with rabbit PH20 set forth in SEQ ID NO:24. FIG. 2H depicts the alignment of a human soluble PH20 set forth in SEQ ID NO:3 with guinea pig PH20 set forth in SEQ ID NO:29. FIG. 2I depicts the alignment of a human soluble PH20 set forth in SEQ ID NO:3 with Fox PH20 set forth in SEQ ID NO:31. FIG. 2J depicts the alignment of a human soluble PH20 set forth in SEQ ID NO:3 with Gibbon PH20 set forth in SEQ ID NO:387. FIG. 2K depicts the alignment of a human soluble PH20 set forth in SEQ ID NO:3 with Marmoset PH20 set forth in SEQ ID NO:389. FIG. 2L depicts the alignment of a human soluble PH20 set forth in SEQ ID NO:3 with Orangutan PH20 set forth in SEQ ID NO:391.

### DETAILED DESCRIPTION

### Outline

A. Definitions
B. PH20 Hyaluronidase and Thermal Stability
   1. Structure
      Soluble PH20 Polypeptides
   2. Function
   3. Thermal Stability of PH20 Hyaluronidases
C. Modified PH20 Polypeptides: Uber-Thermophiles
   1. Exemplary Amino Acid Replacements
   2. Nucleic Acid Molecules
   3. Additional Modifications and Conjugates
      a. Decreased Immunogenicity
      b. Conjugation to Polymers
D. Methods for Identifying Modified Thermally Stable Hyaluronan-Degrading Enzymes
   1. Hyaluronan-Degrading Enzymes and Libraries of Modified Hyaluronan-Degrading Enzymes
   2. Screening or Testing for Activity Under Thermal Stress Conditions
   3. Selection or Identification
   4. Iterative Methods
E. Production of Modified Polypeptides and Encoding Nucleic Acid Molecules
   1. Isolation or Preparation of Nucleic Acids Encoding PH20 Polypeptides
   2. Generation of Mutant or Modified Nucleic Acid and Encoding Polypeptides
   3. Vectors and Cells
   4. Expression
      a. Prokaryotic Cells
      b. Yeast Cells
      c. Insects and Insect Cells
      d. Mammalian expression
      e. Plants and plant cells
   5. Purification
   6. Modification of Polypeptides by PEGylation
F. Pharmaceutical Compositions and Formulations, Dosages and Administration
   1. Formulations (liquids, injectables, solutions and emulsions)
   a. Lyophilized
   b. Exemplary Formulations
      i. pH and Buffer
      ii. Salt (e.g. NaCl)
      iii. Preservatives
      iv. Stabilizers
   2. Compositions for Other Routes of Administration
   3. Dosages and Administration
   4. Combinations and Co-Formulations with Therapeutic Agents
   5. Packaging, Articles of Manufacture and Kits
G. Methods of Assessing Hyaluronidase Activity
   1. Hyaluronidase Activity
   2. Thermal Stability
   Solubility
   3. Other Assays to Assess Stability
   4. Solubility
   5. Pharmacodynamics/Pharmacokinetics
H. Methods of Treatment and Combination Therapy
   1. Methods of Delivering Therapeutic Agents
   2. Methods of Treating Hyaluronan-Associated Disease and Conditions
   3. Other Uses
I. Examples

### A. DEFINITIONS

Unless defined otherwise, all technical and scientific terms used herein have the same meaning as is commonly understood by one of skill in the art to which the invention(s) belong. All patents, patent applications, published applications and publications, Genbank sequences, databases, websites and other published materials referred to throughout the entire disclosure herein, unless noted otherwise, are incorporated by reference in their entirety. In the event that there are a plurality of definitions for terms herein, those in this section prevail. Where reference is made to a URL or other such identifier or address, it understood that such identifiers can change and particular information on the internet can come and go, but equivalent information can be found by searching the internet. Reference thereto evidences the availability and public dissemination of such information.

As used herein, a hyaluronan-degrading enzyme refers to an enzyme that catalyzes the cleavage of a hyaluronan polymer (also including hyaluronic acid; (HA)) into smaller molecular weight fragments. Exemplary hyaluronan-degrading enzymes are hyaluronidases, and also include particular chondroitinases and lyases that have the ability to depolymerize a hyaluronan polymer. Exemplary chondroitinases that are hyaluronan-degrading enzymes include, but are not limited to, chondroitin ABC lyase (also known as chondroitinase ABC), chondroitin AC lyase (also known as chondroitin sulfate lyase or chondroitin sulfate eliminase) and chondroitin C lyase. Chondroitin ABC lyase contains two enzymes, chondroitin-sulfate-ABC endolyase (EC 4.2.2.20) and chondroitin-sulfate-ABC exolyase (EC 4.2.2.21). An exemplary chondroitin-sulfate-ABC endolyases and chondroitin-sulfate-ABC exolyases include, but are not limited to, those from Proteus vulgaris and Pedobacter heparinus (the Proteus vulgaris chondroitin-sulfate-ABC endolyase is set forth in SEQ ID NO:452; Sato et al. (1994) Appl. Microbiol. Biotechnol. 41(1):39-46). Exemplary chondroitinase AC enzymes from bacteria include, but are not limited to, those from Pedobacter heparinus, set forth in SEQ ID NO: 453, Victivallis vadensis, set forth in SEQ ID NO:454, and Arthrobacter aurescens (Tkalec et al. (2000) Applied and Environmental Microbiology 66(1):29-35; Ernst et al. (1995) Critical Reviews in Biochemistry and Molecular Biology 30(5):387-444). Exemplary chondroitinase C enzymes from bacteria include, but are not limited to, those from Streptococcus and Flavobacterium (Hibi et al. (1989) FEMS-Microbiol-Lett. 48(2):121-4; Michelacci et al. (1976) J. Biol. Chem. 251:1154-8; Tsuda et al. (1999) Eur. J. Biochem. 262:127-133).

As used herein, hyaluronidase area hyaluronan degrading enzymes and refers to a class of enzymes hyaluronan degrading enzymes that degrade hyaluronan. Hyaluronidases include, but are not limited to, bacterial hyaluronidases (EC 4.2.2.1 or EC 4.2.99.1), hyaluronidases from leeches, other parasites and crustaceans (EC 3.2.1.36), and mammalian-type hyaluronidases (EC 3.2.1.35). Hyaluronidases include any of non-human origin including, but not limited to, murine, canine, feline, leporine, avian, bovine, ovine, porcine, equine, piscine, ranine, bacterial, and any from leeches, other parasites, and crustaceans. Exemplary human hyaluronidases include HYAL1, HYAL2, HYAL3, HYAL4, and PH20. Also included amongst hyaluronidases are soluble hyaluronidases, including, ovine and bovine PH20, and soluble PH20. Exemplary hyaluronidases include any set forth in SEQ ID NOS: 6, 7-31, 69, 70, 71, 72, 387-392, 399-451, mature forms thereof (lacking the signal sequence), or allelic or species variants thereof. Hyaluronidases also include truncated forms thereof that exhibit hyaluronidase activity, including C-terminal truncated variants that are soluble.

As used herein, PH20 refers to a type of hyaluronidase that occurs in sperm and is neutral-active. PH-20 occurs on the sperm surface, and in the lysosome-derived acrosome, where it is bound to the inner acrosomal membrane. PH20 includes those of any origin including, but not limited to, human, chimpanzee, Cynomolgus monkey, Rhesus monkey, murine, bovine, ovine, guinea pig, rabbit and rat origin. Exemplary PH20 polypeptides, including precursor and mature forms, include those from human (SEQ ID NOS:6 and 7), chimpanzee (SEQ ID NOS:8, 9, 10, 399 and 400), Rhesus monkey (SEQ ID NOS:11 and 12), Cynomolgus monkey (SEQ ID NOS:13 and 14), cow (e.g., SEQ ID NOS:15-18); mouse (SEQ ID NOS:19 and 20); rat (SEQ ID NOS:21 and 22); rabbit (SEQ ID NOS:23 and 24); sheep (SEQ ID NOS:25-27), guinea pig (SEQ ID NOS:28 and 29); fox (SEQ ID NOS: 30 and 31); Gibbon (SEQ ID NOS:387 and 388), Marmoset (SEQ ID NOS:389 and 390) and orangutan (SEQ ID NOS:391 and 392). Reference to PH20 includes precursor PH20 polypeptides and mature PH20 polypeptides (such as those in which a signal sequence has been removed), truncated forms thereof that have activity, and includes allelic variants and species variants, variants encoded by splice variants, and other variants, including polypeptides that have at least 40%, 45%, 50%, 55%, 65%, 70%, 75%, 80%, 85%, 90%, 95%, 96%, 97%, 98%, 99% or more sequence identity to the precursor polypeptides set forth in SEQ ID NO:7, or the mature forms thereof. PH20 polypeptides also include those that contain chemical or posttranslational modifications and those that do not contain chemical or posttranslational modifications. Such modifications include, but are not limited to, PEGylation, albumination, glycosylation, farnysylation, carboxylation, hydroxylation, phosphorylation, and other polypeptide modifications known in the art. Examples of commercially available bovine or ovine soluble hyaluronidases are Vitrase® hyaluronidase (ovine hyaluronidase) and Amphadase® hyaluronidase (bovine hyaluronidase).

As used herein, a soluble PH20 refers to a polypeptide characterized by its solubility under physiological conditions. Generally, a soluble PH20 lacks all or a portion of a glycophosphatidyl anchor (GPI) attachment sequence, or does not otherwise sufficiently anchor to the cell membrane. For example, a soluble PH20 can be a C-terminally truncated variant of a PH20 lacking a contiguous sequence of amino acids that corresponds to all or a portion of a glycophosphatidyl anchor (GPI) attachment sequence. Upon expression in a cell, a soluble PH20 does not become membrane anchored and is secreted into the medium. Soluble PH20 proteins can be distinguished, for example, by its partitioning into the aqueous phase of a Triton X-114 solution warmed to 37° C. (Bordier et al., (1981) J. Biol. Chem., 256:1604-7). Membrane-anchored, such as lipid anchored hyaluronidases, will partition into the detergent rich phase, but will partition into the detergent-poor or aqueous phase following treatment with Phospholipase-C. Included among soluble PH20 hyaluronidases are membrane anchored hyaluronidases in which one or more regions associated with anchoring of the hyaluronidase to the membrane has been removed or modified, where the soluble form retains hyaluronidase activity. Soluble hyaluronidases include recombinant soluble hyaluronidases and those contained in or purified from natural sources, such as, for example, testes extracts from sheep or cows. Exemplary of such soluble hyaluronidases are soluble human PH20 (SEQ ID NO: 3 or 32-66). Other soluble hyaluronidases include ovine (SEQ ID NO:25-27) and bovine (SEQ ID NO:16 or 18) PH20.

As used herein, a soluble human PH20 (sHuPH20) includes human PH20 polypeptides that lack a contiguous sequence of amino acids from the C-terminus of a human PH20 such that all or a portion of the glycosylphosphatidylinositol (GPI) anchor sequence (C-terminally truncated PH20 polypeptides) is missing whereby, if expressed in a cell, the polypeptides are secreted, and/or are soluble under physiological conditions. For example, soluble human PH20 polypeptides include C-terminally truncated polypeptides of the human PH20 set forth as SEQ ID NO:6 in its precursor form or in SEQ ID NO:7 in its mature form lacking the signal sequence, or allelic variants thereof (e.g. set forth in any of SEQ ID NOS: 68-72). Solubility can be assessed by any suitable method that demonstrates solubility under physiologic conditions. Exemplary of such methods is the Triton® X-114 assay, that assesses partitioning into the aqueous phase and that is described above. In addition, a soluble human PH20 polypeptide is, if produced in CHO cells, such as CHO-S cells, a polypeptide that is expressed and is secreted into the cell culture medium. Soluble human PH20 polypeptides, however, are not limited to those produced in CHO cells, but can be produced in any cell or by any method, including recombinant expression and polypeptide synthesis. Reference to secretion by CHO cells is definitional. Hence, if a polypeptide could be expressed and secreted by CHO cells and is soluble in the media, i.e., partitions into the aqueous phase when extracted with Triton® X-114, it is a soluble PH20 polypeptide whether or not it is so-produced. The precursor polypeptides for sHuPH20 polypeptides can include a signal sequence, such as a heterologous or non-heterologous (i.e., native) signal sequence. Exemplary of the precursors are those that include a signal sequence, such as the native 35 amino acid signal sequence at amino acid positions 1-35 (see, e.g., amino acids 1-35 of SEQ ID NO:6).

As used herein, “native” or “wildtype” with reference to a PH20 polypeptide refers to a PH20 polypeptide encoded by a native or naturally occurring PH20 gene, including allelic variants, that is present in an organism, including a human and other animals, in nature. Reference to wild-type PH20 without reference to a species is intended to encompass any species of a wild-type PH20. Included among wild-type PH20 polypeptides are the encoded precursor polypeptide, fragments thereof, and processed forms thereof, such as a mature form lacking the signal peptide as well as any pre- or post-translationally processed or modified forms thereof. Also included among native PH20 polypeptides are those that are post-translationally modified, including, but not limited to, those that are modified by glycosylation, carboxylation and/or hydroxylation. The amino acid sequences of exemplary wild-type human PH20 are set forth in SEQ ID NOS: 6 and 7 and those of allelic variants, including mature forms thereof, are set forth in SEQ ID NOS:68-72. Other animals produce native PH20, including, but not limited to, native or wildtype sequences set forth in any of SEQ ID NOS: 8-31, 387-392, 399 or 400.

As used herein, modification refers to modification of a sequence of amino acid residues of a polypeptide or a sequence of nucleotides in a nucleic acid molecule and includes deletions, insertions, and replacements of amino acids and nucleotides, respectively. Modifications also can include post-translational modifications or other changes to the molecule that can occur due to conjugation or linkage, directly or indirectly, to another moiety. Methods of modifying a polypeptide are routine to those of skill in the art, such as by using recombinant DNA methodologies.

As used herein, “deletion,” when referring to modification of a nucleic acid or polypeptide sequence, refers to the deletion of one or more nucleotides or amino acids compared to a sequence, such as a target polynucleotide or polypeptide or a native or wild-type sequence.

As used herein, “insertion” when referring to modification of a nucleic acid or amino acid sequence, describes the inclusion of one or more additional nucleotides or amino acids, within a target, native, wild-type or other related sequence. Thus, a nucleic acid molecule that contains one or more insertions compared to a wild-type sequence, contains one or more additional nucleotides within the linear length of the sequence. As used herein, “additions,” to nucleic acid and amino acid sequences describe addition of nucleotides or amino acids onto either termini compared to another sequence.

As used herein, “substitution” or “replacement” with respect to a modification refers to the replacing of one or more nucleotides or amino acids in a native, target, wild-type or other nucleic acid or polypeptide sequence with an alternative nucleotide or amino acid, without changing the length (as described in numbers of residues) of the molecule. Thus, one or more substitutions in a molecule does not change the number of amino acid residues or nucleotides of the molecule Amino acid replacements compared to a particular polypeptide can be expressed in terms of the number of the amino acid residue along the length of the polypeptide sequence or a reference polypeptide sequence. For example, a modified polypeptide having a modification in the amino acid at the 19^{th} position of the amino acid sequence that is a substitution of Isoleucine (Ile; I) for cysteine (Cys; C) can be expressed as “replacement with Cys or C at a position corresponding to position 19,” I19C, Ile19Cys, or simply C19, to indicate that the amino acid at the modified 19^{th} position is a cysteine. In this example, the molecule having the substitution has a modification at Ile 19 of the unmodified polypeptide.

As used herein, a “modified hyaluronan-degrading enzyme” refers to a hyaluronan-degrading enzyme that contains a modification compared to a reference or unmodified hyaluronan-degrading enzyme. The modification can be an amino acid replacement (substitution), insertion (addition) or deletion of one or more amino acid residues. The amino acid residue can be a natural or non-natural amino acid. In some cases, the modification can be a post-translational modification. A modified hyaluronan-degrading enzyme can have up to 150 amino acid differences compared to a reference or unmodified hyaluronan-degrading enzyme, so long as the resulting modified hyaluronan-degrading enzyme exhibits hyaluronidase activity. Typically, a modified hyaluronan-degrading enzyme contains 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acid modifications.

As used herein, an unmodified hyaluronan-degrading enzyme refers to a starting polypeptide that is selected for modification as provided herein. The starting polypeptide can be a naturally-occurring, wild-type form of a polypeptide. In addition, the starting polypeptide can be altered or mutated, such that it differs from a native wild type isoform but is nonetheless referred to herein as a starting unmodified polypeptide relative to the subsequently modified polypeptides produced herein to have the properties described herein. Thus, existing proteins known in the art that have been modified to have a desired increase or decrease in a particular activity or property compared to an unmodified reference protein can be selected and used as the starting unmodified polypeptide. For example, a protein that has been modified from its native form by one or more single amino acid changes and possesses either an increase or decrease in a desired property, such as a change in an amino acid residue or residues to alter glycosylation, can be selected for modification, and hence referred to herein as unmodified, for further modification. An unmodified hyaluronan-degrading enzyme includes human and non-human hyaluronan-degrading enzymes, including hyaluronan-degrading enzymes from non-human mammals and bacteria. Exemplary unmodified hyaluronan-degrading enzyme are any set forth in SEQ ID NOS: 2, 3, 6, 7-66, 68-72, 387-392, 399-454 or mature, C-terminally truncated forms thereof that exhibit hyaluronidase activity, or a hyaluronan-degrading enzyme that exhibits at least 75%, 80%, 85%, 86%, 87%, 88%, 89%, 90%, 91%, 92%, 93%, 94%, 95%, 96%, 97%, 98%, or 99% sequence identity to any of SEQ ID NOS: 2, 3, 6, 7-66, 68-72, 387-392, 399-454. It is understood that an unmodified hyaluronan-degrading enzyme generally is one that does not contain the modification(s), such as amino acid replacement(s) of a modified hyaluronan-degrading enzyme.

As used herein, “modified PH20 polypeptide” or “variant PH20 polypeptide” refers to a PH20 polypeptide that contains at least one amino acid modification, such as at least one amino acid replacement as described herein, in its sequence of amino acids compared to a reference unmodified PH20 polypeptide. A modified PH20 polypeptide can have up to 150 amino acid replacements, so long as the resulting modified PH20 polypeptide exhibits hyaluronidase activity. Typically, a modified PH20 polypeptide contains 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acid replacements. It is understood that a modified PH20 polypeptide also can include any one or more other modifications, in addition to at least one amino acid replacement as described herein.

As used herein, an unmodified PH20 polypeptide refers to a starting PH20 polypeptide that is selected for modification as provided herein. The starting polypeptide can be a naturally-occurring, wild-type form of a polypeptide. In addition, the starting polypeptide can be altered or mutated, such that it differs from a native wild type isoform but is nonetheless referred to herein as a starting unmodified polypeptide relative to the subsequently modified polypeptides produced herein. Thus, existing proteins known in the art that have been modified to have a desired increase or decrease in a particular activity or property compared to an unmodified reference protein can be selected and used as the starting unmodified polypeptide. For example, a protein that has been modified from its native form by one or more single amino acid changes and possesses either an increase or decrease in a desired property, such as a change in an amino acid residue or residues to alter glycosylation, can be selected for modification, and hence referred to herein as unmodified, for further modification. Exemplary unmodified PH20 polypeptides is a human PH20 polypeptide or allelic or species variants thereof or other variants, including mature and precursor polypeptides. For example, exemplary reference PH20 polypeptides is a mature full length PH20 polypeptide set forth in SEQ ID NOS: 7, 69 or 72, or in C-terminally truncated forms thereof such as set forth in any of SEQ ID NOS: 3 and 32-66, or in a PH20 polypeptide that exhibits at least 68%, 69%, 70%, 75%, 80%, 85%, 86%, 87%, 88%, 89%, 90%, 91%, 92%, 93%, 94%, 95%, 96%, 97%, 98%, or 99% sequence identity to any of SEQ ID NOS: 3, 7, 32-66, 69 or 72. A reference PH20 polypeptide also can include the corresponding precursor form such as set forth in any of SEQ ID NOS: 2, 6, 68, 70 or 71 or other precursor forms, or in a PH20 polypeptide that exhibits at least 68%, 69%, 70%, 75%, 80%, 85%, 86%, 87%, 88%, 89%, 90%, 91%, 92%, 93%, 94%, 95%, 96%, 97%, 98%, or 99% sequence identity to any of SEQ ID NOS: 2, 6, 68, 70 or 71. It is understood that an unmodified hyaluronan-degrading enzyme generally is one that does not contain the modification(s), such as amino acid replacement(s) of a modified hyaluronan-degrading enzyme.

As used herein, an N-linked moiety refers to an asparagine (N) amino acid residue of a polypeptide that is capable of being glycosylated by post-translational modification of a polypeptide. Exemplary N-linked moieties of human PH20 include amino acids N47, N131, N200, N219, N333 and N358 of the sequence of amino acids set forth in SEQ ID NO: 3 or 7 (corresponding to amino acid residues N82, N166, N235, N254, N368 and N393 of human PH20 set forth in SEQ ID NO: 6).

As used herein, an N-glycosylated polypeptide refers to a PH20 polypeptide containing oligosaccharide linkage of at least three N-linked amino acid residues, for example, N-linked moieties corresponding to amino acid residues N200, N333 and N358 of SEQ ID NO:3 or 7. An N-glycosylated polypeptide can include a polypeptide where three, four, five and up to all of the N-linked moieties are linked to an oligosaccharide. The N-linked oligosaccharides can include oligomannose, complex, hybrid or sulfated oligosaccharides, or other oligosaccharides and monosaccharides.

As used herein, an N-partially glycosylated polypeptide refers to a polypeptide that minimally contains an N-acetylglucosamine glycan linked to at least three N-linked moieties. A partially glycosylated polypeptide can include various glycan forms, including monosaccharides, oligosaccharides, and branched sugar forms, including those formed by treatment of a polypeptide with EndoH, EndoF1, EndoF2 and/or EndoF3.

As used herein, uber-thermophile with reference to a PH20 polypeptide refers to a PH20 polypeptide variant that exhibits at least 50% of its hyaluronidase activity at 52° C. for 10 minutes compared to its activity 4° C. For example, an uber-thermophile refers to a PH20 polypeptide variant that that has a T₅₀ at 10 minutes as determined in a thermal challenge assay of at least or about at least or 52° C. For example, an uber-thermophile can exhibit at least 60%, 65%, 70%, 75%, 80%, 85%, 90%, 95% or more of the activity at 52° C. for 10 minutes compared to its activity at 4° C. An uber-thermophile also generally exhibits at least 40% of the hyaluronidase activity of the corresponding enzyme without the modification(s) or wildype PH20 (e.g. a human PH20 or soluble C-terminal truncated fragment thereof set forth in any of SEQ ID NOS: 3, 7 or 32-66) at 4° C., and greater or increased activity at 52° C. than the same enzyme without the modification(s) and/or wildype PH20 (e.g. a human PH20 or soluble C-terminal truncated fragment thereof set forth in any of SEQ ID NOS: 3, 7 or 32-66). An uber-thermophile also includes PH20 polypeptides that exhibit at least 50% hyaluronidase activity at temperatures greater than 52° C. Thus, the T₅₀ of an uber-thermophile as determined in a thermal challenge assay at 10 minutes can be 52° C., or greater than 52° C., such as greater than 53° C., 54° C., 55° C., 56° C., 57° C., 58° C., 59° C., 60° C., 61° C., 62° C., 63° C., 64° C., 65° C. or greater.

As used herein, property refers to a physical or structural property, such as the three-dimensional structure, pI, half-life, conformation and other such physical characteristics. For example, a change in a property can be manifested as the solubility, aggregation or crystallization of a protein.

As used herein, “protein stability” refers to a measure of the maintenance of one or more physical properties of a protein in response to an environmental condition (e.g. an elevated temperature). In one embodiment, the physical property is the maintenance of the covalent structure of the protein (e.g. the absence of proteolytic cleavage, unwanted oxidation or deamidation). In another embodiment, the physical property is the presence of the protein in a properly folded state (e.g. the absence of soluble or insoluble aggregates or precipitates). In one embodiment, stability of a protein is measured by assaying a biophysical property of the protein, for example thermal stability, pH unfolding profile, stable removal of glycosylation, solubility, biochemical function (e.g., ability to bind to a protein (e.g., a ligand, a receptor, an antigen, etc.) or chemical moiety, etc.), and/or combinations thereof. In another embodiment, biochemical function is demonstrated by the binding affinity of an interaction. Stability can be measured using methods known in the art and/or described herein.

As used herein, an elevated temperature is a temperature that is or is greater than room temperature (e.g. generally greater than 25° C.). Generally, an elevated temperature is a temperature that is at least, greater than, or about 30° C., such as 30° C. to 42° C., and generally 32° C. to 37° C. or 35° C. to 37° C., inclusive.

As used herein, “stability” or “stable” with reference to a modified PH20 polypeptide or modified hyaluronan-degrading enzyme means that it retains some activity in the presence of an elevated temperature, such as at least 20%, 30%, 40%, 50%, 60%, 70%, 80%, 90% or more of the original or initial hyaluronidase activity prior to exposure to the elevated temperature. Generally, a modified PH20 hyaluronidase is stable if it retains at least 50% or more of the hyaluronidase activity after incubation at an elevated temperature or exposure to an elevated temperature compared to incubation or exposure to a permissive temperature such as a refrigerated temperature (e.g. 2° C.-8° C.). Assays to assess hyaluronidase activity are known to one of skill in the art and described herein. It is understood that the stability of the enzyme need not be permanent or long term, but is manifested for a duration of time in which activity is desired. For example, a modified PH20 hyaluronidase is stable if it exhibits an activity for at least 2 hours, 3 hours, 4 hours, 6 hours, 12 hours, 24 hours, one day, two days, three days, four days, five days, six days, one week, one month, six months or one year upon exposure, or during exposure, to an elevated temperature.

As used herein, thermal stability refers to the measure of the resistance to denaturation of polypeptide that occurs upon exposure to high or elevated temperatures, and hence is the ability of a protein to function at a particular temperature. A polypeptide is thermally stable at a temperature if it retains at least 50%, 55%, 60%, 65%, 70%, 75%, 80%, 85%, 90%, 95% or more of an activity or a property of the polypeptide at the temperature. Thermal stability can be measured either by known procedures or by the methods described herein. In certain embodiments, thermal stability is evaluated by measuring the melting temperature (Tm) of a protein or by a thermal challenge assay (Tc).

As used herein, the melting temperature (Tm; also called transition temperature) is the temperature at the midpoint of a thermal transition curve where 50% of molecules of a composition are in a folded state. Hence, it is the temperature at which 50% of a macromolecule becomes denatured, and is a standard parameter for describing the thermal stability of a protein. Methods to determine Tm are well-known to a skilled artisan and include, for example, analytical spectroscopy methods such as, but are not limited to, differential scanning calorimetry (DSC), circular dicroism (CD) spectroscopy), fluorescence emission spectroscopy or nuclear magnetic resonance (NMR) spectroscopy.

As used herein, a “thermal challenge” assay (or a “thermal gradient” assay) refers to an assay performed by incubation of a protein at a range of temperatures for a set period of time and testing for an activity (e.g. hyaluronidase activity). A thermal challenge assay can be used to determine the temperature for a tested time period at which 50% activity is retained, which is the T₅₀ value (also called the Tc value) for the tested time period. A thermal challenge assay can be performed at any desired time period, and is user determined. As used herein, a thermal stress condition refers to a temperature condition in which an unmodified hyaluronan-degrading enzyme or other reference hyaluronan-degrading enzyme (e.g. wildtype or native) is susceptible to denaturation or degradation, and thus is not stable. For purposes herein, a thermal stress condition is typically a temperature that is or is greater than the melting temperature (Tm) or the T₅₀ value as determined in a thermal challenge assay of an unmodified hyaluronan-degrading enzyme or other reference hyaluronan-degrading enzyme (e.g. wildtype or native). For example, the thermal stress condition can be a temperature that is or is more than 0.5° C., 1° C., 2° C., 3° C., 4° C., 5° C., 6° C., 7° C., 8° C., 9° C., 10° C., 15° C., 20° C. or higher.

As used herein, “solubility” with reference to a protein refers to a protein that is homogenous in an aqueous solution, whereby protein molecules diffuse and do not sediment spontaneously. Hence a soluble protein solution is one in which there is an absence of a visible or discrete particle in a solution containing the protein, such that the particles cannot be easily filtered. Generally, a protein is soluble if there are no visible or discrete particles in the solution. For example, a protein is soluble if it contains no or few particles that can be removed by a filter with a pore size of 0.22 μm.

As used herein, aggregation or crystallization with reference to a protein refers to the presence of visible or discrete particles in a solution containing the protein. Typically, the particles are greater than 10 μm in size, such as greater than 15 μm, 20 μm, 25 μm, 30 μm, 40 μm, 50 μm or greater. Aggregation or crystallization can arise due to reduced solubility, increased denaturation of a protein or the formation of covalent bonds.

As used herein, “increased temperature resistance” or “increased temperature stability” refers to any amount of increased resistance to denaturation caused by elevated temperature of a modified hyalruonan-degrading enzyme (e.g. modified PH20) compared to a corresponding hyaluronan-degrading enzyme not containing the modification. For example, increased temperature resistance can be manifested as an increased thermal stability, such as an increased (i.e. higher) Tm or T₅₀, of the modified hyaluronan-degrading enzyme (e.g. modified PH20) compared to the corresponding hyaluronan-degrading enzyme not containing the modification. In other examples, denaturation is associated with or causes increased crystallization or aggregation, reduced solubility or decreased activity. Hence, resistance to denaturation means that the protein exhibits decreased aggregation or crystallization, increased solubility or increased or greater activity (e.g., hyaluronidase activity) when exposed to a denaturing condition compared to a reference protein (e.g. unmodified enzyme or a protein without the modification(s) that confers the increased resistance/stability). The increased temperature resistance need not be absolute or permanent, but can be achieved because the denaturation of the modified hyaluronan-degrading enzyme occurs more slowly than the unmodified enzyme at the elevated temperature such that an activity or property of the modified hyaluronan-degrading enzyme is achieved for longer. For example, a modified hyaluronan-degrading enzyme, such as a modified PH20 hyaluronidase, exhibits increased temperature resistance if it exhibits, for example, at least or about at least 1%, 2%, 3%, 4%, 5%, 6%, 7%, 8%, 9%, 10%, . . . 20%, . . . 30%, . . . 40%, . . . 50%, . . . 60%, . . . , 70%, . . . 80%, . . . 90%, 91%, 92%, 93%, 94%, 95%, 96%, 97%, 98%, 99%, or 100% more resistance to an elevated temperature than the corresponding unmodified polypeptide to the same temperature. In some instances, a modified polypeptide exhibits 105%, 110%, 120%, 130%, 140%, 150%, 200%, 300%, 400%, 500%, or more increased temperature resistance compared to an unmodified polypeptide. Hence, a modified PH20 hyaluronidase exhibits increased temperature stability if it exhibits at least or about at least 110%, 120%, 130%, 140%, 150%, 160%, 170%, 180%, 190%, 200%, 250%, 300%, 400%, 500%, 600%, 700%, 800%, 900%, 1000% or more of the activity of the unmodified or reference PH20 hyaluronidase when exposed to an elevated temperature for a period of time.

As used herein, activity refers to a functional activity or activities of a polypeptide or portion thereof associated with a full-length (complete) protein. Functional activities include, but are not limited to, biological activity, catalytic or enzymatic activity, antigenicity (ability to bind or compete with a polypeptide for binding to an anti-polypeptide antibody), immunogenicity, ability to form multimers, and the ability to specifically bind to a receptor or ligand for the polypeptide.

As used herein, hyaluronidase activity refers to the ability to enzymatically catalyze the cleavage of hyaluronic acid (also named hyaluronan). For example, for a human hyaluronan-degrading enzyme, such as a human PH20, hyaluronidase activity refers to the ability to enzymatically catalyze the cleavage of human hyaluronic acid. The United States Pharmacopeia (USP) XXII assay for hyaluronidase determines hyaluronidase activity indirectly by measuring the amount of higher molecular weight hyaluronic acid, or hyaluronan (HA), substrate remaining after the enzyme is allowed to react with the HA for 30 min at 37° C. (USP XXII-NF XVII (1990) 644-645 United States Pharmacopeia Convention, Inc, Rockville, Md.). A Reference Standard solution can be used in an assay to ascertain the relative activity, in units, of any hyaluronidase. In vitro assays to determine the hyaluronidase activity of hyaluronidases, such as PH20, including modified PH20 polypeptides, are known in the art and described herein. Exemplary assays include the microturbidity assay described herein that measures cleavage of hyaluronic acid by hyaluronidase indirectly by detecting the insoluble precipitate formed when the uncleaved hyaluronic acid binds with serum albumin. Reference Standards can be used, for example, to generate a standard curve to determine the activity in Units of the hyaluronidase being tested.

As used herein, neutral active refers to the ability of a PH20 polypeptide to enzymatically catalyze the cleavage of hyaluronic acid at neutral pH, such as at a pH between or about between pH 6.0 to pH 7.8.

As used herein, “refrigeration” with reference to a protein composition refers to storage at a temperature that is 3 to 5° C. (37 to 41° F.).

As used herein “without refrigeration” or “non-refrigerated” with reference to a protein composition refers to storage at room temperature or ambient temperature. The particular conditions and temperatures are not necessarily constant, and can change or are in flux depending on the locale or setting. For example, temperatures can fluctuate during shipping, handling or other use that can occur without refrigeration. Thus, temperatures achieved without refrigeration include continuous, variable or intermittent temperatures. For example, the temperatures in tropical climates can range from 15-42° C. Generally, without refrigeration, a protein composition can be exposed to elevated temperatures at or greater than 25° C. for some period of time, including temperatures that are at least, greater than, or about 30° C., such as 30° C. to 42° C., and generally 32° C. to 37° C. or 35° C. to 37° C., inclusive.

As used herein, “room temperature” refers to a range generally from about or at 18° C. to about or at 32° C., and typically in the range of 20° C. to 25° C. It generally is a temperature that exists in a temperature-controlled building. Those of skill in the art appreciate that room temperature varies by location and prevailing conditions. For example, room temperatures can be higher in warmer climates such as Italy or Texas. Also, room temperatures can vary with season, such that a standard room temperature in summer (e.g. 23° C. to 26° C.) can differ from winter (e.g. 19° C. to 21° C.).

As used herein, “ambient temperature” refers to the temperature of the surroundings, such as occurs during shipping, handling, and other storage of a protein composition. Hence, the ambient temperature can vary within a range from below 0° C. to 42° C. For indoor climates, an ambient temperature can be the same as the room temperature. For outdoor climates, an ambient temperature can be cooler or warmer than the room temperature. Those of skill in the art will appreciate that the ambient temperature varies by location and prevailing conditions. In tropical climates, the ambient temperatures is generally warmer than other climates. The summer ambient temperature is generally warmer than the winter ambient temperature.

As used herein, a summer ambient temperature reflects temperature extremes that can be encountered during the summer months (e.g. May to September or August to July) such as can occur between the latitudes of 59.9° north and 37.8° south. For example, such temperatures can range from 23° C. to 39° C.

As used herein, “tropical climate” refers to the climate in the tropic regions near the equator (e.g. such as can occur between the latitudes 23.5° south and 23.5° north) where the mean temperature for all twelve months is typically greater than 18° C., and can be much higher in some cases. For example, in tropical regions like Thar Desert in India, prevailing heat conditions in May and June can be in the range of 46° C. to 50° C. for 5-6 hours per day. Hence, reference to a tropical climate refers to temperatures in the range of 22° C. to 50° C., and generally a daytime temperature of 30° C. to 42° C.

As used herein, recitation that proteins are “compared under the same conditions” means that different proteins are treated identically or substantially identically such that any one or more conditions that can influence the activity or properties of a protein or agent are not varied or not substantially varied between the test agents. For example, when the hyaluronidase activity of a modified PH20 polypeptide is compared to an unmodified PH20 polypeptide any one or more conditions such as the amount or concentration of the polypeptide; presence, including amount, of excipients, carriers or other components in a formulation other than the active agent (e.g., modified PH20 hyaluronidase); temperature; time of storage; storage vessel; properties of storage (e.g., agitation) and/or other conditions associated with exposure or use are identical or substantially identical between and among the compared polypeptides. Generally, for purposes herein, when comparing proteins only the temperature is varied or different.

As used herein, “predetermined time” refers to a time that is established or decided in advance. For example, the predetermined time can be a time chosen in advance that is associated with the desired duration of activity of a hyaluronan-degrading enzyme depending on the desired application or use of the protein. A predetermined time can be hours, days, months or years. For example, a predetermined time can be at least about or about 2 hours, 3 hours, 4 hours, 5 hours, 6 hours, 12 hours, 24 hours, 2 days, 3 days, 4 days, 5 days, 6 days, one week, two weeks, three weeks, one month, six months, one year or more.

As used herein, “storage” means that a formulation is not immediately administered to a subject once prepared, but is kept for a period of time under particular conditions (e.g., particular temperature, time, and/or form (e.g., liquid or lyophilized form)) prior to use. For example, a liquid formulation can be kept or exposed for a period of time (e.g. days or months) prior to administration to a subject to varied temperatures such as refrigerated (0° C. to 10° C., such as 2° C. to 8° C.), room temperature (e.g., temperature up to 32° C., such as 18° C. to about or at 32° C.), or other ambient temperatures that are elevated (e.g., 30° C. to 42° C., such as 32° C. to 37° C. or 35° C. to 37° C.).

As used herein, an “excipient” refers to a compound in a formulation of an active agent that does not provide the biological effect of the active agent when administered in the absence of the active agent. Exemplary excipients include, but are not limited to, salts, buffers, stabilizers, tonicity modifiers, metals, polymers, surfactants, preservatives, amino acids and sugars.

As used herein, a stabilizing agent or stabilizer refers to compound added to the formulation to protect the modified PH20 polypeptide or other active agent from degradation, if necessary, such as due to denaturation conditions to which a formulation herein is exposed when handled, stored or used. Thus, included are agents that prevent proteins from degradation from other components in the compositions. Exemplary of such agents are amino acids, amino acid derivatives, amines, sugars, polyols, salts and buffers, surfactants, inhibitors or substrates, proteins (e.g. albumin) and other agents as described herein.

As used herein, a “buffer” or “buffering agent” refers to a substance, generally a solution, that can keep its pH constant, despite the addition of strong acids or strong bases and external influences of temperature, pressure, volume or redox potential. A buffer prevents change in the concentration of another chemical substance, e.g., proton donor and acceptor systems that prevent marked changes in hydrogen ion concentration (pH). The pH values of all buffers are temperature and concentration dependent. The choice of buffer to maintain a pH value or range can be empirically determined by one of skill in the art based on the known buffering capacity of known buffers. Exemplary buffers include but are not limited to, bicarbonate buffer, cacodylate buffer, phosphate buffer or Tris buffer. For example, Tris buffer (tromethamine) is an amine based buffer that has a pKa of 8.06 and has an effective pH range between 7.9 and 9.2. For Tris buffers, pH increases about 0.03 unit per ° C. temperature decrease, and decreases 0.03 to 0.05 unit per ten-fold dilution.

As used herein, the residues of naturally occurring α-amino acids are the residues of those 20 α-amino acids found in nature which are incorporated into protein by the specific recognition of the charged tRNA molecule with its cognate mRNA codon in humans.

As used herein, nucleic acids include DNA, RNA and analogs thereof, including peptide nucleic acids (PNA) and mixtures thereof. Nucleic acids can be single or double-stranded. When referring to probes or primers, which are optionally labeled, such as with a detectable label, such as a fluorescent or radiolabel, single-stranded molecules are contemplated. Such molecules are typically of a length such that their target is statistically unique or of low copy number (typically less than 5, generally less than 3) for probing or priming a library. Generally a probe or primer contains at least 14, 16 or 30 contiguous nucleotides of sequence complementary to or identical to a gene of interest. Probes and primers can be 10, 20, 30, 50, 100 or more nucleic acids long.

As used herein, a peptide refers to a polypeptide that is from 2 to 40 amino acids in length.

As used herein, the amino acids which occur in the various sequences of amino acids provided herein are identified according to their known, three-letter or one-letter abbreviations (Table 1). The nucleotides which occur in the various nucleic acid fragments are designated with the standard single-letter designations used routinely in the art.

As used herein, an “amino acid” is an organic compound containing an amino group and a carboxylic acid group. A polypeptide contains two or more amino acids. For purposes herein, amino acids include the twenty naturally-occurring amino acids, non-natural amino acids and amino acid analogs (i.e., amino acids wherein the α-carbon has a side chain).

As used herein, “amino acid residue” refers to an amino acid formed upon chemical digestion (hydrolysis) of a polypeptide at its peptide linkages. The amino acid residues described herein are presumed to be in the “L” isomeric form. Residues in the “D” isomeric form, which are so designated, can be substituted for any L-amino acid residue as long as the desired functional property is retained by the polypeptide. NH₂ refers to the free amino group present at the amino terminus of a polypeptide. COOH refers to the free carboxy group present at the carboxyl terminus of a polypeptide. In keeping with standard polypeptide nomenclature described in J. Biol. Chem., 243: 3557-3559 (1968), and adopted 37 C.F.R. §§1.821-1.822, abbreviations for amino acid residues are shown in Table 1:

TABLE 1
Table of Correspondence

SYMBOL

| 1-Letter | 3-Letter | AMINO ACID |
|---|---|---|
| Y | Tyr | Tyrosine |
| G | Gly | Glycine |
| F | Phe | Phenylalanine |
| M | Met | Methionine |
| A | Ala | Alanine |
| S | Ser | Serine |
| I | Ile | Isoleucine |
| L | Leu | Leucine |
| T | Thr | Threonine |
| V | Val | Valine |
| P | Pro | Proline |
| K | Lys | Lysine |
| H | His | Histidine |
| Q | Gln | Glutamine |
| E | Glu | Glutamic Acid |
| Z | Glx | Glu and/or Gln |
| W | Trp | Tryptophan |
| R | Arg | Arginine |
| D | Asp | Aspartic Acid |
| N | Asn | Asparagine |
| B | Asx | Asn and/or Asp |
| C | Cys | Cysteine |
| X | Xaa | Unknown or Other |

It should be noted that all amino acid residue sequences represented herein by formulae have a left to right orientation in the conventional direction of amino-terminus to carboxyl-terminus. In addition, the phrase “amino acid residue” is broadly defined to include the amino acids listed in the Table of Correspondence (Table 1) and modified and unusual amino acids, such as those referred to in 37 C.F.R. §§1.821-1.822, and incorporated herein by reference. Furthermore, it should be noted that a dash at the beginning or end of an amino acid residue sequence indicates a peptide bond to a further sequence of one or more amino acid residues, to an amino-terminal group such as NH₂ or to a carboxyl-terminal group such as COON.

As used herein, “naturally occurring amino acids” refer to the 20 L-amino acids that occur in polypeptides.

As used herein, “non-natural amino acid” refers to an organic compound that has a structure similar to a natural amino acid but has been modified structurally to mimic the structure and reactivity of a natural amino acid. Non-naturally occurring amino acids thus include, for example, amino acids or analogs of amino acids other than the 20 naturally-occurring amino acids and include, but are not limited to, the D-stereoisomers of amino acids. Exemplary non-natural amino acids are described herein and are known to those of skill in the art.

As used herein, an isokinetic mixture is one in which the molar ratios of amino acids has been adjusted based on their reported reaction rates (see, e.g., Ostresh et al., (1994) Biopolymers 34:1681).

As used herein, suitable conservative substitutions of amino acids are known to those of skill in the art and can be made generally without altering the biological activity of the resulting molecule. Those of skill in the art recognize that, in general, single amino acid substitutions in non-essential regions of a polypeptide do not substantially alter biological activity (see, e.g., Watson et al. Molecular Biology of the Gene, 4th Edition, 1987, The Benjamin/Cummings Pub. co., p. 224). Such substitutions can be made in accordance with those set forth in TABLE 2 as follows:

TABLE 2

| | Exemplary conservative |
|---|---|
| Original residue | substitution |
| Ala (A) | Gly; Ser |
| Arg (R) | Lys |
| Asn (N) | Gln; His |
| Cys (C) | Ser |
| Gln (Q) | Asn |
| Glu (E) | Asp |
| Gly (G) | Ala; Pro |
| His (H) | Asn; Gln |
| Ile (I) | Leu; Val |
| Leu (L) | Ile; Val |
| Lys (K) | Arg; Gln; Glu |
| Met (M) | Leu; Tyr; Ile |
| Phe (F) | Met; Leu; Tyr |
| Ser (S) | Thr |
| Thr (T) | Ser |
| Trp (W) | Tyr |
| Tyr (Y) | Trp; Phe |
| Val (V) | Ile; Leu |

Other substitutions also are permissible and can be determined empirically or in accord with known conservative substitutions.

As used herein, a DNA construct is a single or double stranded, linear or circular DNA molecule that contains segments of DNA combined and juxtaposed in a manner not found in nature. DNA constructs exist as a result of human manipulation, and include clones and other copies of manipulated molecules.

As used herein, a DNA segment is a portion of a larger DNA molecule having specified attributes. For example, a DNA segment encoding a specified polypeptide is a portion of a longer DNA molecule, such as a plasmid or plasmid fragment, which, when read from the 5′ to 3′ direction, encodes the sequence of amino acids of the specified polypeptide.

As used herein, the term polynucleotide means a single- or double-stranded polymer of deoxyribonucleotides or ribonucleotide bases read from the 5′ to the 3′ end. Polynucleotides include RNA and DNA, and can be isolated from natural sources, synthesized in vitro, or prepared from a combination of natural and synthetic molecules. The length of a polynucleotide molecule is given herein in terms of nucleotides (abbreviated “nt”) or base pairs (abbreviated “bp”). The term nucleotides is used for single- and double-stranded molecules where the context permits. When the term is applied to double-stranded molecules it is used to denote overall length and will be understood to be equivalent to the term base pairs. It will be recognized by those skilled in the art that the two strands of a double-stranded polynucleotide can differ slightly in length and that the ends thereof can be staggered; thus all nucleotides within a double-stranded polynucleotide molecule cannot be paired. Such unpaired ends will, in general, not exceed 20 nucleotides in length.

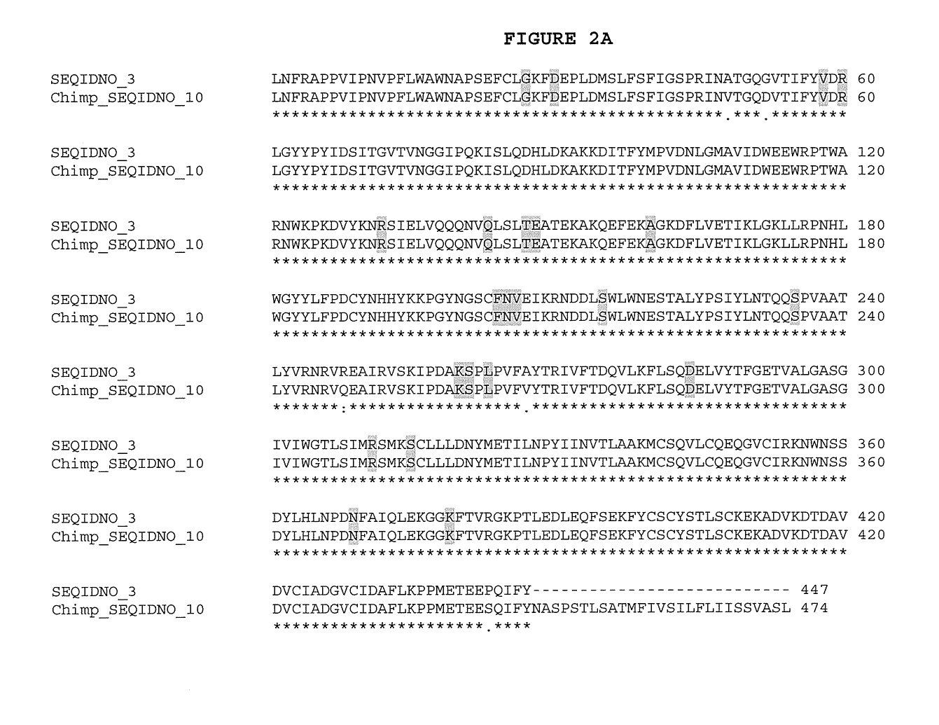

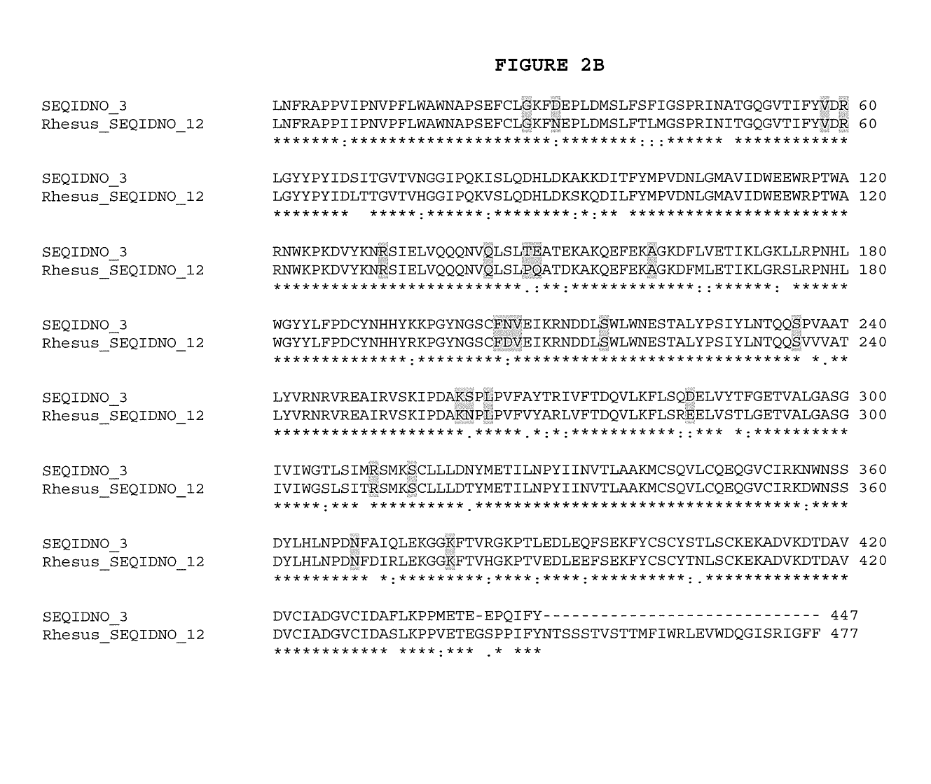

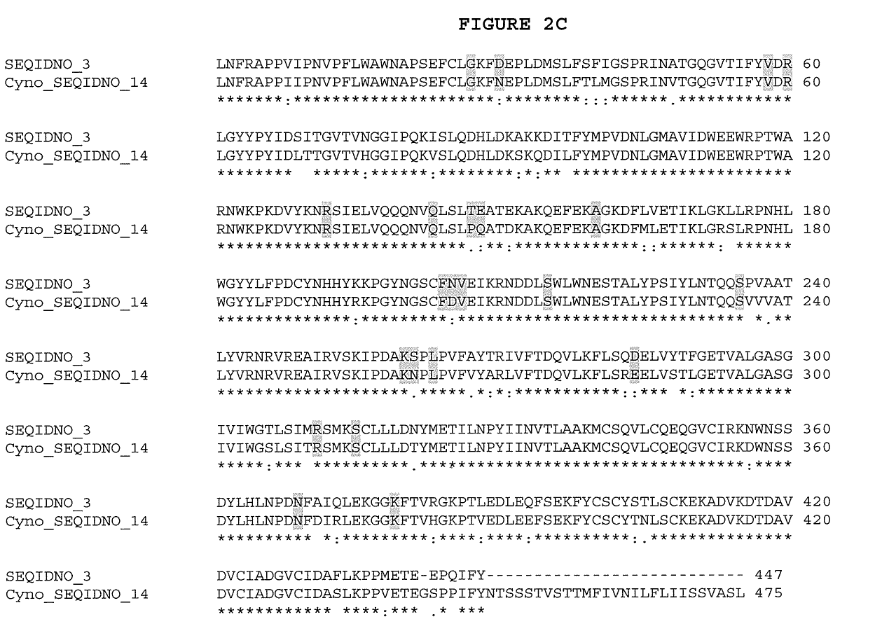

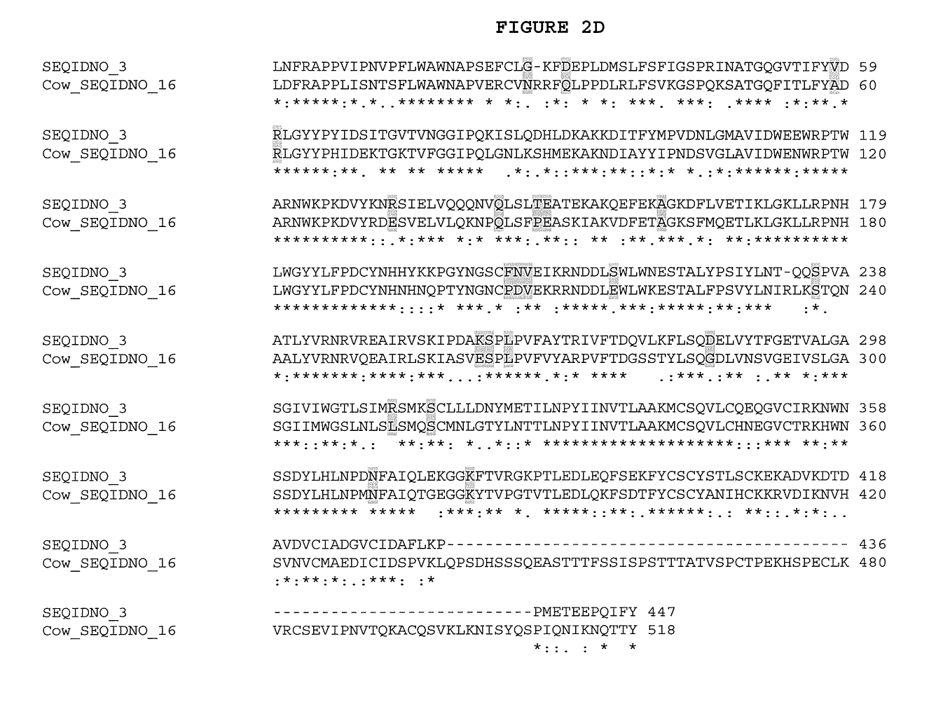

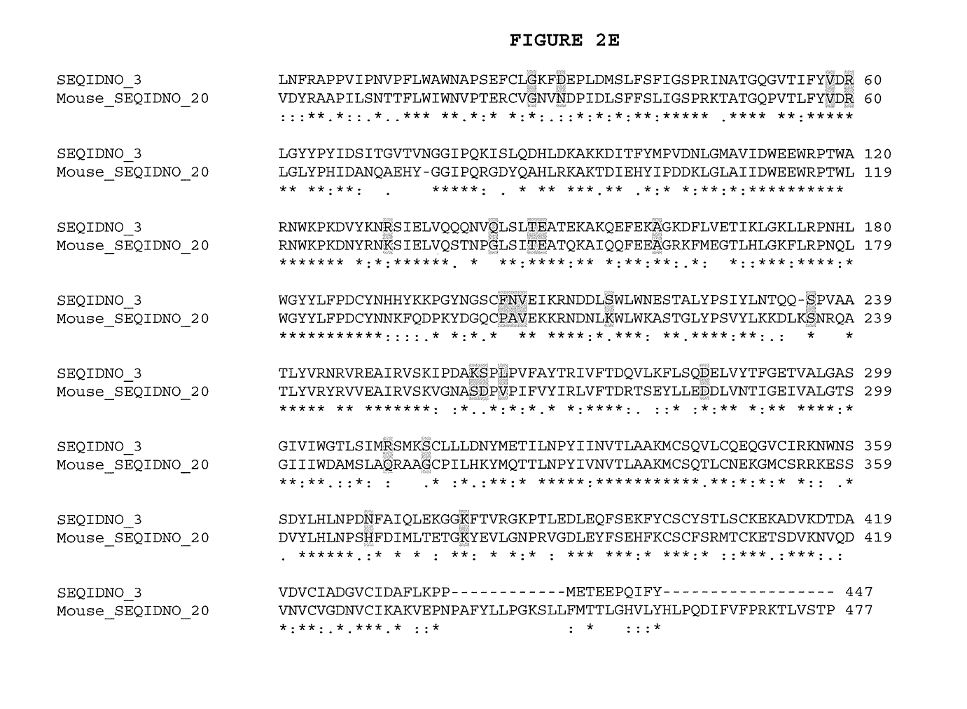

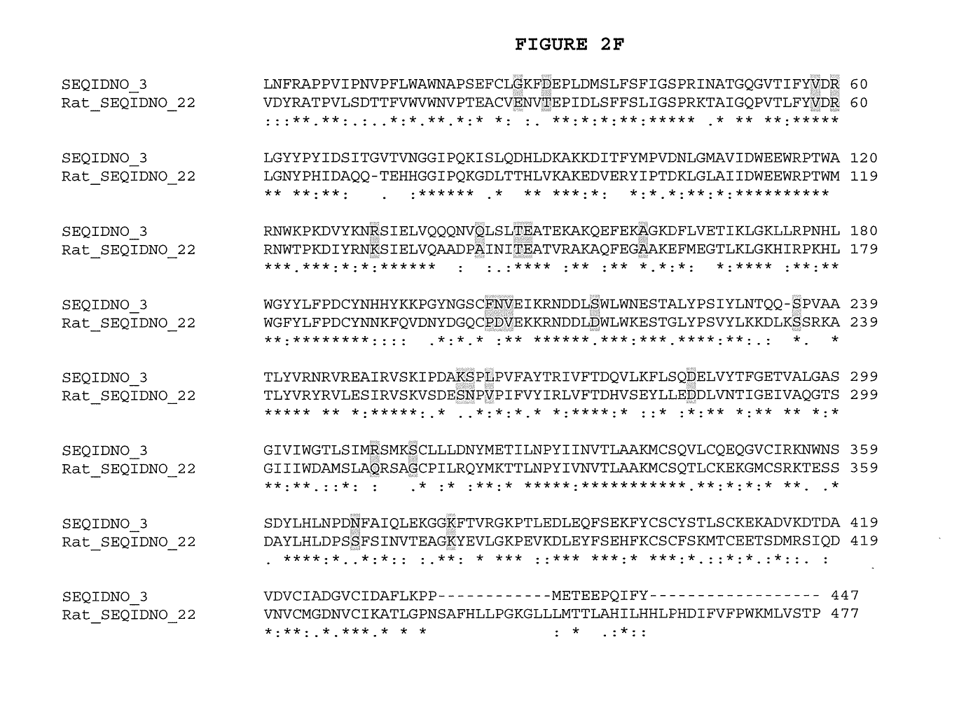

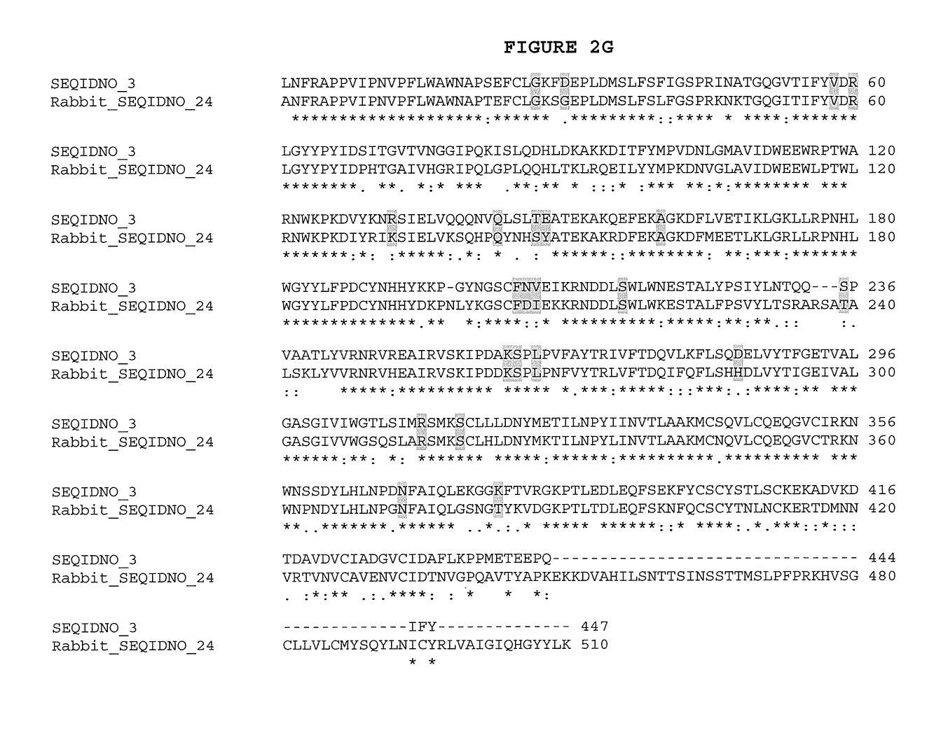

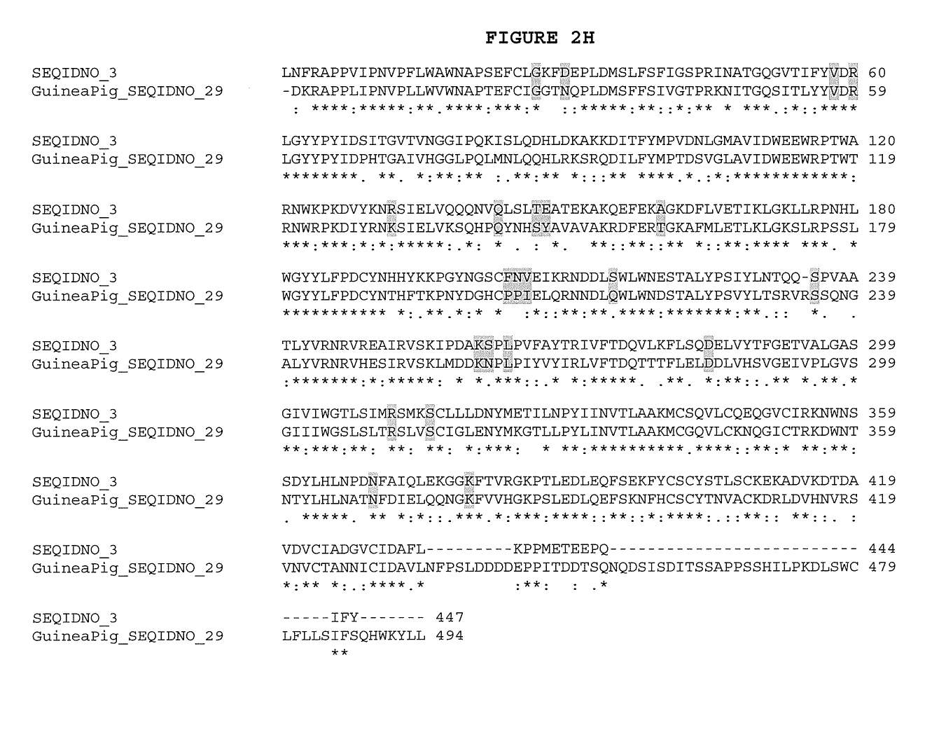

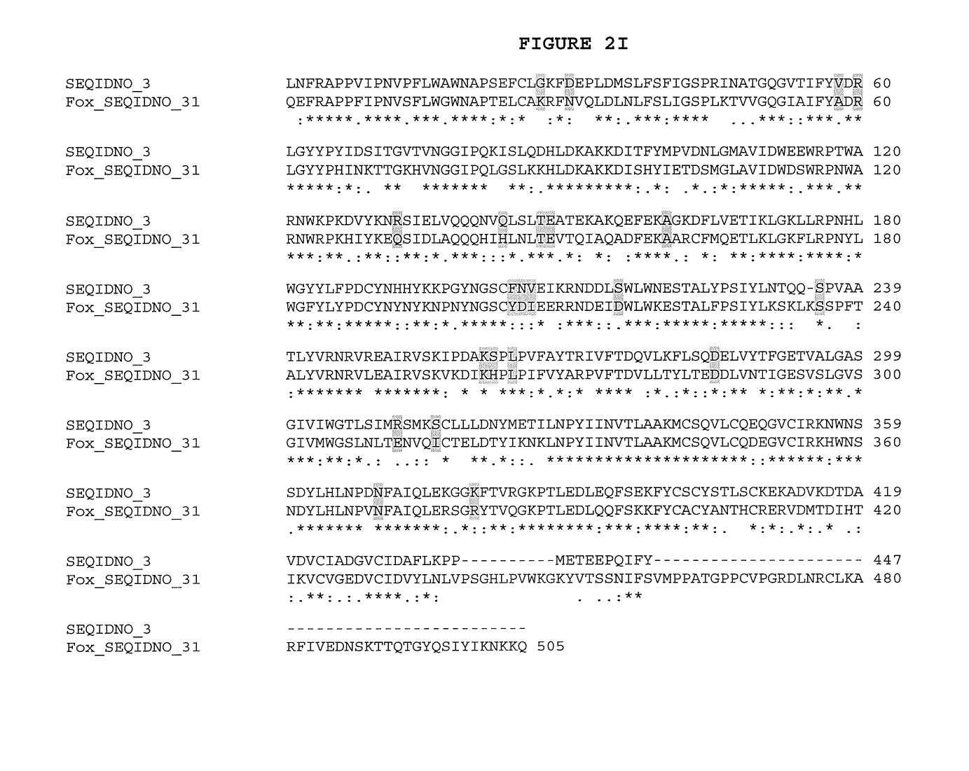

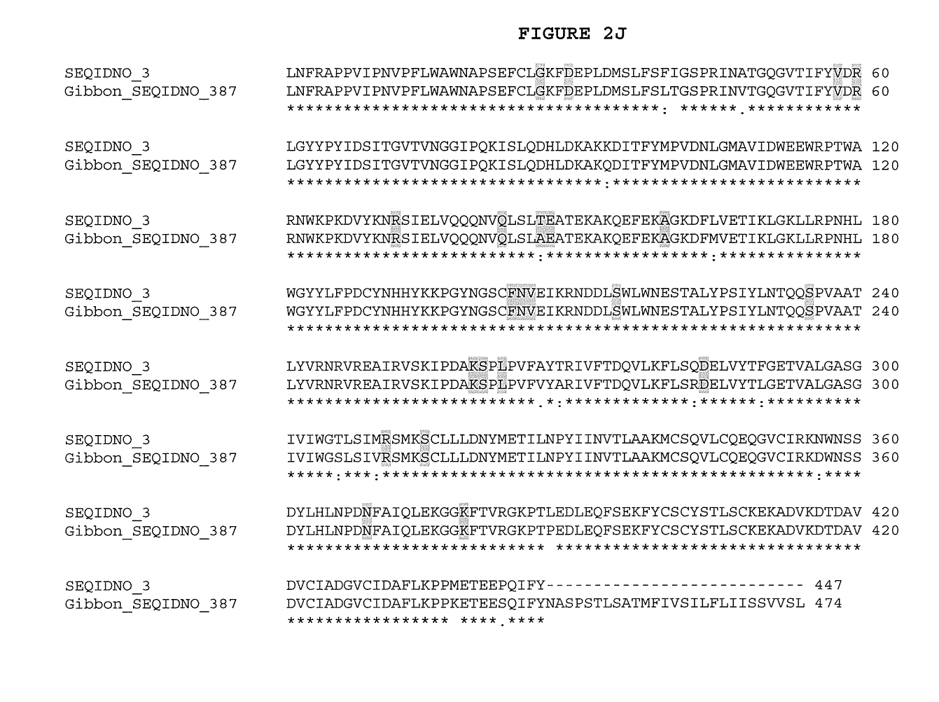

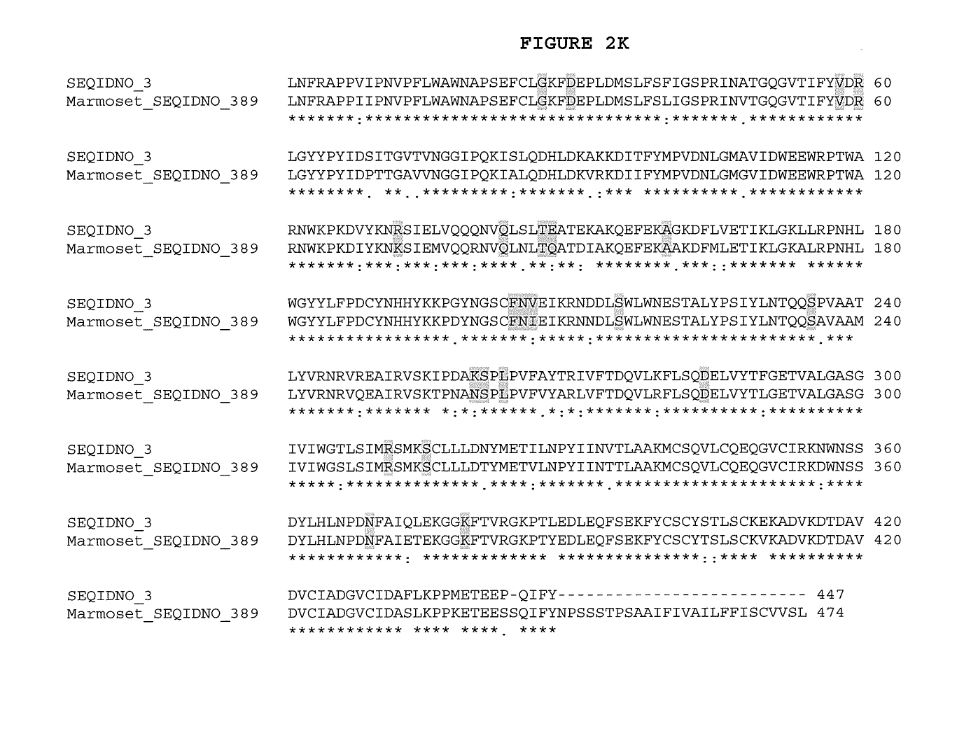

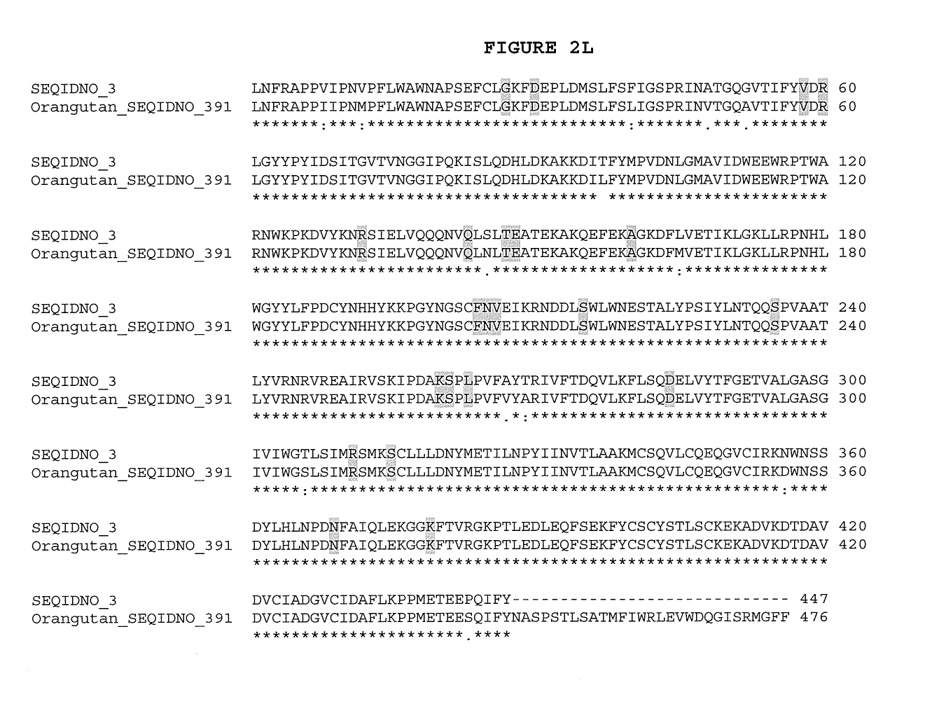

As used herein, “at a position corresponding to” or recitation that nucleotides or amino acid positions “correspond to” nucleotides or amino acid positions in a disclosed or reference sequence, such as set forth in the Sequence listing, refers to nucleotides or amino acid positions identified upon alignment with the disclosed sequence to maximize identity using a standard alignment algorithm, such as the GAP algorithm. SEQ ID NO:3 is an exemplary reference sequence herein. Reference herein that a position or amino acid replacement corresponds to positions with reference to SEQ ID NO:3 also means that the position or amino acid replacement corresponds to positions with reference to any of SEQ ID NOS: 7 or 32-66, since the sequences therein are identical to the corresponding residues as set forth in SEQ ID NO:3. Thus, for purposes herein, alignment of a PH20 sequence is to the amino acid sequence set forth in any of SEQ ID NOS: 3, 7 or 32-66, and in particular SEQ ID NO:3. By aligning the sequences, one skilled in the art can identify corresponding residues, for example, using conserved and identical amino acid residues as guides. In general, to identify corresponding positions, the sequences of amino acids are aligned so that the highest order match is obtained (see, e.g.: Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, N.J., 1994; Sequence Analysis in Molecular Biology, von Heinje, G., Academic Press, 1987; and Sequence Analysis Primer, Gribskov, M. and Devereux, J., eds., M Stockton Press, New York, 1991; Carrillo et al. (1988) SIAM J Applied Math 48:1073). FIG. 2 (A-L) exemplifies exemplary alignments and identification of exemplary corresponding residues for replacement.

As used herein, “sequence identity” refers to the number of identical or similar amino acids or nucleotide bases in a comparison between a test and a reference polypeptide or polynucleotide. Sequence identity can be determined by sequence alignment of nucleic acid or protein sequences to identify regions of similarity or identity. For purposes herein, sequence identity is generally determined by alignment to identify identical residues. Alignment can be local or global, but for purposes herein alignment is generally a global alignment where the full-length of each sequence is compared. Matches, mismatches and gaps can be identified between compared sequences. Gaps are null amino acids or nucleotides inserted between the residues of aligned sequences so that identical or similar characters are aligned. Generally, there can be internal and terminal gaps. Sequence identity can be determined by taking into account gaps as the number of identical residues/length of the shortest sequence×100. When using gap penalties, sequence identity can be determined with no penalty for end gaps (e.g., terminal gaps are not penalized). Alternatively, sequence identity can be determined without taking into account gaps as the number of identical positions/length of the total aligned sequence×100.

As used herein, a “global alignment” is an alignment that aligns two sequences from beginning to end, aligning each letter in each sequence only once. An alignment is produced, regardless of whether or not there is similarity or identity between the sequences. For example, 50% sequence identity based on “global alignment” means that in an alignment of the full sequence of two compared sequences each of 100 nucleotides in length, 50% of the residues are the same. It is understood that global alignment also can be used in determining sequence identity even when the length of the aligned sequences is not the same. The differences in the terminal ends of the sequences will be taken into account in determining sequence identity, unless the “no penalty for end gaps” is selected. Generally, a global alignment is used on sequences that share significant similarity over most of their length. Exemplary algorithms for performing global alignment include the Needleman-Wunsch algorithm (Needleman et al. J. Mol. Biol. 48: 443 (1970)). Exemplary programs for performing global alignment are publicly available and include the Global Sequence Alignment Tool available at the National Center for Biotechnology Information (NCBI) website (ncbi.nlm.nih.gov/), and the program available at deepc2.psi.iastate.edu/aat/align/align.html.

As used herein, a “local alignment” is an alignment that aligns two sequence, but only aligns those portions of the sequences that share similarity or identity. Hence, a local alignment determines if sub-segments of one sequence are present in another sequence. If there is no similarity, no alignment will be returned. Local alignment algorithms include BLAST or Smith-Waterman algorithm (Adv. Appl. Math. 2: 482 (1981)). For example, 50% sequence identity based on “local alignment” means that in an alignment of the full sequence of two compared sequences of any length, a region of similarity or identity of 100 nucleotides in length has 50% of the residues that are the same in the region of similarity or identity.

For purposes herein, sequence identity can be determined by standard alignment algorithm programs used with default gap penalties established by each supplier. Default parameters for the GAP program can include: (1) a unary comparison matrix (containing a value of 1 for identities and 0 for non identities) and the weighted comparison matrix of Gribskov et al. Nucl. Acids Res. 14: 6745 (1986), as described by Schwartz and Dayhoff, eds., Atlas of Protein Sequence and Structure, National Biomedical Research Foundation, pp. 353-358 (1979); (2) a penalty of 3.0 for each gap and an additional 0.10 penalty for each symbol in each gap; and (3) no penalty for end gaps. Whether any two nucleic acid molecules have nucleotide sequences or any two polypeptides have amino acid sequences that are at least 80%, 85%, 90%, 95%, 96%, 97%, 98% or 99% “identical,” or other similar variations reciting a percent identity, can be determined using known computer algorithms based on local or global alignment (see e.g., wikipedia.org/wiki/Sequence_alignment_software, providing links to dozens of known and publicly available alignment databases and programs). Generally, for purposes herein sequence identity is determined using computer algorithms based on global alignment, such as the Needleman-Wunsch Global Sequence Alignment tool available from NCBI/BLAST (blast.ncbi.nlm.nih.gov/Blast.cgi?CMD=Web&Page_TYPE=BlastHome); LAlign (William Pearson implementing the Huang and Miller algorithm (Adv. Appl. Math. (1991) 12:337-357)); and program from Xiaoqui Huang available at deepc2.psi.iastate.edu/aat/align/align.html. Generally, when comparing nucleotide sequences herein, an alignment with penalty for end gaps is used. Local alignment also can be used when the sequences being compared are substantially the same length.

Therefore, as used herein, the term “identity” represents a comparison or alignment between a test and a reference polypeptide or polynucleotide. In one non-limiting example, “at least 90% identical to” refers to percent identities from 90 to 100% relative to the reference polypeptide or polynucleotide. Identity at a level of 90% or more is indicative of the fact that, assuming for exemplification purposes a test and reference polypeptide or polynucleotide length of 100 amino acids or nucleotides are compared, no more than 10% (i.e., 10 out of 100) of amino acids or nucleotides in the test polypeptide or polynucleotide differs from that of the reference polypeptides. Similar comparisons can be made between a test and reference polynucleotides. Such differences can be represented as point mutations randomly distributed over the entire length of an amino acid sequence or they can be clustered in one or more locations of varying length up to the maximum allowable, e.g., 10/100 amino acid difference (approximately 90% identity). Differences also can be due to deletions or truncations of amino acid residues. Differences are defined as nucleic acid or amino acid substitutions, insertions or deletions. Depending on the length of the compared sequences, at the level of homologies or identities above about 85-90%, the result can be independent of the program and gap parameters set; such high levels of identity can be assessed readily, often without relying on software.

As used herein, an allelic variant or allelic variation references any of two or more alternative forms of a gene occupying the same chromosomal locus. Allelic variation arises naturally through mutation, and can result in phenotypic polymorphism within populations. Gene mutations can be silent (no change in the encoded polypeptide) or can encode polypeptides having altered amino acid sequence. The term “allelic variant” also is used herein to denote a protein encoded by an allelic variant of a gene. Typically the reference form of the gene encodes a wildtype form and/or predominant form of a polypeptide from a population or single reference member of a species. Typically, allelic variants, which include variants between and among species typically have at least 80%, 90% or greater amino acid identity with a wildtype and/or predominant form from the same species; the degree of identity depends upon the gene and whether comparison is interspecies or intraspecies. Generally, intraspecies allelic variants have at least about 80%, 85%, 90% or 95% or greater identity with a wildtype and/or predominant form, including 96%, 97%, 98%, 99% or greater identity with a wildtype and/or predominant form of a polypeptide. Reference to an allelic variant herein generally refers to variations in proteins among members of the same species.

As used herein, “allele,” which is used interchangeably herein with “allelic variant” refers to alternative forms of a gene or portions thereof. Alleles occupy the same locus or position on homologous chromosomes. When a subject has two identical alleles of a gene, the subject is said to be homozygous for that gene or allele. When a subject has two different alleles of a gene, the subject is said to be heterozygous for the gene. Alleles of a specific gene can differ from each other in a single nucleotide or several nucleotides, and can include modifications such as substitutions, deletions and insertions of nucleotides. An allele of a gene also can be a form of a gene containing a mutation.

As used herein, species variants refer to variants in polypeptides among different species, including different mammalian species, such as mouse and human. Exemplary of species variants provided herein are primate PH20, such as, but not limited to, human, chimpanzee, macaque, cynomolgus monkey, gibbon, orangutan, or marmoset. Generally, species variants have 70%, 75%, 80%, 85%, 90%, 91%, 92%, 93%, 94%, 95%, 96%, 97%, or 98% sequence identity. Corresponding residues between and among species variants can be determined by comparing and aligning sequences to maximize the number of matching nucleotides or residues, for example, such that identity between the sequences is equal to or greater than 95%, equal to or greater than 96%, equal to or greater than 97%, equal to or greater than 98% or equal to greater than 99%. The position of interest is then given the number assigned in the reference nucleic acid molecule. Alignment can be effected manually or by eye, particularly where sequence identity is greater than 80%.

As used herein, substantially pure means sufficiently homogeneous to appear free of readily detectable impurities, as determined by standard methods of analysis, such as thin layer chromatography (TLC), gel electrophoresis and high performance liquid chromatography (HPLC), used by those of skill in the art to assess such purity, or sufficiently pure such that further purification would not detectably alter the physical and chemical properties, such as enzymatic and biological activities, of the substance. Methods for purification of the compounds to produce substantially chemically pure compounds are known to those of skill in the art. A substantially chemically pure compound can, however, be a mixture of stereoisomers or isomers. In such instances, further purification might increase the specific activity of the compound.

As used herein, isolated or purified polypeptide or protein or biologically-active portion thereof is substantially free of cellular material or other contaminating proteins from the cell or tissue from which the protein is derived, or substantially free from chemical precursors or other chemicals when chemically synthesized. Preparations can be determined to be substantially free if they appear free of readily detectable impurities as determined by standard methods of analysis, such as thin layer chromatography (TLC), gel electrophoresis and high performance liquid chromatography (HPLC), used by those of skill in the art to assess such purity, or sufficiently pure such that further purification would not detectably alter the physical and chemical properties, such as enzymatic and biological activities, of the substance. Methods for purification of the compounds to produce substantially chemically pure compounds are known to those of skill in the art. A substantially chemically pure compound, however, can be a mixture of stereoisomers. In such instances, further purification might increase the specific activity of the compound.

Hence, reference to a substantially purified polypeptide, such as a substantially purified PH20 polypeptide refers to preparations of PH20 proteins that are substantially free of cellular material, includes preparations of proteins in which the protein is separated from cellular components of the cells from which it is isolated or recombinantly-produced. In one embodiment, the term substantially free of cellular material includes preparations of enzyme proteins having less than about 30% (by dry weight) of non-enzyme proteins (also referred to herein as contaminating proteins), generally less than about 20% of non-enzyme proteins or 10% of non-enzyme proteins or less than about 5% of non-enzyme proteins. When the enzyme protein is recombinantly produced, it also is substantially free of culture medium, i.e., culture medium represents less than about or at 20%, 10% or 5% of the volume of the enzyme protein preparation.

As used herein, the term substantially free of chemical precursors or other chemicals includes preparations of enzyme proteins in which the protein is separated from chemical precursors or other chemicals that are involved in the synthesis of the protein. The term includes preparations of enzyme proteins having less than about 30% (by dry weight), 20%, 10%, 5% or less of chemical precursors or non-enzyme chemicals or components.

As used herein, synthetic, with reference to, for example, a synthetic nucleic acid molecule or a synthetic gene or a synthetic peptide refers to a nucleic acid molecule or polypeptide molecule that is produced by recombinant methods and/or by chemical synthesis methods.

As used herein, production by recombinant means or using recombinant DNA methods means the use of the well known methods of molecular biology for expressing proteins encoded by cloned DNA.

As used herein, vector (or plasmid) refers to discrete elements that are used to introduce a heterologous nucleic acid into cells for either expression or replication thereof. The vectors typically remain episomal, but can be designed to effect integration of a gene or portion thereof into a chromosome of the genome. Also contemplated are vectors that are artificial chromosomes, such as yeast artificial chromosomes and mammalian artificial chromosomes. Selection and use of such vehicles are well known to those of skill in the art.

As used herein, an expression vector includes vectors capable of expressing DNA that is operatively linked with regulatory sequences, such as promoter regions, that are capable of effecting expression of such DNA fragments. Such additional segments can include promoter and terminator sequences, and optionally can include one or more origins of replication, one or more selectable markers, an enhancer, a polyadenylation signal, and the like. Expression vectors are generally derived from plasmid or viral DNA, or can contain elements of both. Thus, an expression vector refers to a recombinant DNA or RNA construct, such as a plasmid, a phage, recombinant virus or other vector that, upon introduction into an appropriate host cell, results in expression of the cloned DNA. Appropriate expression vectors are well known to those of skill in the art and include those that are replicable in eukaryotic cells and/or prokaryotic cells and those that remain episomal or those which integrate into the host cell genome.

As used herein, vector also includes “virus vectors” or “viral vectors.” Viral vectors are engineered viruses that are operatively linked to exogenous genes to transfer (as vehicles or shuttles) the exogenous genes into cells. Viral vectors include, but are not limited to, adenoviral vectors, retroviral vectors and vaccinia virus vectors.

As used herein, “operably” or “operatively linked” when referring to DNA segments means that the segments are arranged so that they function in concert for their intended purposes, e.g., transcription initiates downstream of the promoter and upstream of any transcribed sequences. The promoter is usually the domain to which the transcriptional machinery binds to initiate transcription and proceeds through the coding segment to the terminator.

As used herein, a conjugate refers to a modified PH20 polypeptide linked directly or indirectly to one or more other polypeptides or chemical moieties. Such conjugates include fusion proteins, those produced by chemical conjugates and those produced by any other method whereby at least one modified PH20 polypeptide is linked, directly or indirectly to another polypeptide or chemical moiety so long as the conjugate retains hyaluronidase activity. Exemplary of conjugates provided herein include PH20 polypeptides linked directly or indirectly to a multimerization domain (e.g. an Fc moiety), a toxin, a label or a drug.

As used herein, a fusion protein refers to a polypeptide encoded by a nucleic acid sequence containing a coding sequence from one nucleic acid molecule and the coding sequence from another nucleic acid molecule in which the coding sequences are in the same reading frame such that when the fusion construct is transcribed and translated in a host cell, the protein is produced containing the two proteins. The two molecules can be adjacent in the construct or separated by a linker polypeptide that contains, 1, 2, 3, or more, but typically fewer than 10, 9, 8, 7, or 6 amino acids. The protein product encoded by a fusion construct is referred to as a fusion polypeptide. Examples of fusion polypeptides include Fc fusions.

As used herein, a polymer that is conjugated to a modified PH20 polypeptide refers to any polymer that is covalently or otherwise stably linked, directly or via a linker, to such polypeptide. Such polymers, typically increase serum half-life, and include, but are not limited to, sialic moieties, polyethylene glycol (PEG) moieties, dextran, and sugar and other moieties, such as for glycosylation.

As used herein, the term assessing or determining is intended to include quantitative and qualitative determination in the sense of obtaining an absolute value for the activity of a product, and also of obtaining an index, ratio, percentage, visual or other value indicative of the level of the activity. Assessment can be direct or indirect.

As used herein, a “composition” refers to any mixture of two or more products or compounds. It can be a solution, a suspension, liquid, powder, a paste, aqueous, non-aqueous, or any combination thereof.

As used herein, a formulation refers to a composition containing at least one active pharmaceutical or therapeutic agent and one or more excipients.

As used herein, a co-formulation refers to a composition containing two or more active or pharmaceutical or therapeutic agents and one or more excipients. For example, a co-formulation of a fast-acting insulin and a hyaluronan degrading enzyme contains a fast-acting insulin, a hyaluronan degrading enzyme, and one or more excipients.

As used herein, “a combination” refers to any association between two or among more items or elements, for example, two or more items that can be used together. Exemplary combinations include, but are not limited to, two or more pharmaceutical compositions, a composition containing two or more active ingredients, such as two modified PH20 polypeptides; a modified PH20 polypeptide and an anticancer agent, such as a chemotherapeutic compound; a modified PH20 polypeptide and a therapeutic agent (e.g. an insulin); a modified PH20 polypeptide and a plurality therapeutic and/or imaging agents, or any association thereof. Such combinations can be packaged as kits.

As used herein, a kit is a packaged combination, optionally, including instructions for use of the combination and/or other reactions and components for such use.

As used herein, a pharmaceutically effective agent or therapeutic agent includes any bioactive agent that can exhibit a therapeutic effect to treat a disease or disorder. Exemplary therapeutic agents are described herein. Therapeutic agents include, but are not limited to, anesthetics, vasoconstrictors, dispersing agents, conventional therapeutic drugs, including small molecule drugs, including, but not limited to, bisphosphonates, and therapeutic proteins, including, but not limited to, insulin, IgG molecules, antibodies, cytokines and coagulation factors.

As used herein, “insulin” refers to a hormone, precursor or a synthetic or recombinant analog thereof that acts to increase glucose uptake and storage and/or decrease endogenous glucose production. Insulin and analogs thereof are well known to one of skill in the art, including in human and allelic and species variants thereof. Insulin is translated as a precursor polypeptide designated preproinsulin (110 amino acid for human insulin), containing a signal peptide that directs the protein to the endoplasmic reticulum (ER) wherein the signal sequence is cleaved, resulting in proinsulin. Proinsulin is processed further to release a C- or connecting chain peptide (a 31 amino acid C-chain in human insulin). The resulting insulin contains an A-chain (21 amino acid in length in human insulin; set forth in SEQ ID NO:393) and a B-chain (30 amino acid in length in human insulin; set forth in SEQ ID NO:394) which are cross-linked by disulfide bonds. A fully cross-linked human insulin contains three disulfide bridges: one between position 7 of the A-chain and position 7 of the B-chain, a second between position 20 of the A-chain and position 19 of the B-chain, and a third between positions 6 and 11 of the A-chain. Reference to an insulin includes monomeric and multimeric insulins, including hexameric insulins, as well as humanized insulins. Exemplary insulin polypeptides are those of mammalian, including human, origin. Reference to insulin includes preproinsulin, proinsulin and insulin polypeptides in single-chain or two-chain forms, truncated forms thereof that have activity, and includes allelic variants and species variants of human insulin, variants encoded by splice variants, and other variants, such as insulin analogs. An exemplary insulin is human insulin having a sequence of amino acids of the A- and B-chains of human insulin are set forth in SEQ ID NOS: 393 and 394, respectively, and variants or analogs thereof that exhibit at least 80%, 85%, 90%, 91%, 92%, 93%, 94%, 95%, 96%, 97%, 98% or 99% sequence identity thereto to one or both of the A-chain or B-chain and that acts to increase glucose uptake and storage and/or decrease endogenous glucose production.

As used herein, “fast-acting insulin” refers to any insulin that exhibits peak insulin levels at or about not more than four hours following subcutaneous administration to a subject. Fast-acting insulins include any insulin or any fast-acting insulin composition for acute administration to a diabetic subject in response to an actual, perceived, or anticipated hyperglycemic condition in the subject arising at the time of, or within about four hours following, administration of the fast-acting insulin (such as a prandial hyperglycemic condition resulting or anticipated to result from, consumption of a meal), whereby the fast-acting insulin is able to prevent, control or ameliorate the acute hyperglycemic condition. Fast-acting insulins include recombinant insulins and isolated insulins (also referred to as “regular” insulins) such as the insulin sold as human insulin, porcine insulins and bovine insulins, as well as rapid acting insulin analogs (also termed fast-acting insulin analogs herein) designed to be rapid acting by virtue of amino acid changes. Exemplary regular insulin preparations include, but are not limited to, human regular insulins, such as those sold under the trademarks Humulin® R, Novolin® R and Velosulin®, Insulin Human, USP and Insulin Human Injection, USP, as well as acid formulations of insulin, such as, for example, Toronto Insulin, Old Insulin, and Clear Insulin, and regular pig insulins, such as Iletin II® (porcine insulin) Regular insulins typically have an onset of action of between 30 minutes to an hour, and a peak insulin level of 2-5 hours post administration.

As used herein, rapid acting insulin analogs (also called fast-acting insulin analogs) are insulins that have a rapid onset of action. Rapid insulins typically are insulin analogs that have been engineered, such as by the introduction of one or more amino acid substitutions, to be more rapid acting than regular insulins. Rapid acting insulin analogs typically have an onset of action of 10-30 minutes post injection, with peak insulin levels observed 30-90 minutes post injection. Exemplary rapid acting insulin analogs are analogs of human insulin containing one or more amino acid changes in the A-chain and/or B-chain of human insulin set forth in SEQ ID NO:393 or 394, respectively, and that exhibit an onset of action 10-30 minutes post injection with peak insulin levels observed 30-90 minutes post injection. Exemplary rapid acting insulin analogs include, but are not limited to, for example, insulin lispro (e.g., Humalog® insulin), insulin aspart (e.g., NovoLog® insulin), and insulin glulisine (e.g., Apidra® insulin) the fast-acting insulin composition sold as VIAject® and VIAtab® (see, e.g., U.S. Pat. No. 7,279,457). The amino acid sequence of exemplary rapid acting insulin analogs have an A chain with a sequence of amino acids set forth in SEQ ID NO:393 and a B chain having a sequence of amino acids set forth in any of SEQ ID NOS:395-397. Also included are any other insulins that have an onset of action of 30 minutes or less and a peak level before 90 minutes, typically 30-90 minutes, post injection.

As used herein, a human insulin refers to an insulin that is synthetic or recombinantly produced based upon the human polypeptide, including allelic variants and analogs thereof

As used herein, fast-acting human insulins or human fast-acting insulin compositions include any human insulin or composition of a human insulin that is fast-acting, but excludes non-human insulins, such as regular pig insulin.

As used herein, “disease or disorder” refers to a pathological condition in an organism resulting from cause or condition including, but not limited to, infections, acquired conditions, genetic conditions, and characterized by identifiable symptoms.

As used herein, a hyaluronan-associated disease, disorder or condition refers to any disease or condition in which hyaluronan levels are elevated as cause, consequence or otherwise observed in the disease or condition. Hyaluronan-associated diseases and conditions are associated with elevated hyaluronan expression in a tissue or cell, increased interstitial fluid pressure, decreased vascular volume, and/or increased water content in a tissue. For example, such diseases and conditions include, but are not limited to, including cancers, disc pressure and edema. Exemplary diseases and conditions, include, but are not limited to, hyaluronan-rich cancers, for example, tumors, including solid tumors such as late-stage cancers, metastatic cancers, undifferentiated cancers, ovarian cancer, in situ carcinoma (ISC), squamous cell carcinoma (SCC), prostate cancer, pancreatic cancer, non-small cell lung cancer, breast cancer, colon cancer and other cancers. Exemplary hyaluronan-associated diseases and conditions also are diseases that are associated with elevated interstitial fluid pressure, such as diseases associated with disc pressure, and edema, for example, edema caused by organ transplant, stroke, brain trauma or other injury. Hyaluronan-associated diseases, disorders or conditions can be treated by administration of a composition containing a hyaluronan degrading enzyme, such as a hyaluronidase, for example, a soluble hyaluronidase, either alone or in combination with or in addition to another treatment and/or agent. In one example, treatment of the hyaluronan-associated condition, disease or disorder includes amelioration, reduction, or other beneficial effect on one or more of increased interstitial fluid pressure (IFP), decreased vascular volume, and increased water content in a tissue.

As used herein, “treating” a subject with a disease or condition means that the subject's symptoms are partially or totally alleviated, or remain static following treatment. Hence treatment encompasses prophylaxis, therapy and/or cure. Prophylaxis refers to prevention of a potential disease and/or a prevention of worsening of symptoms or progression of a disease. Treatment also encompasses any pharmaceutical use of a modified interferon and compositions provided herein.

As used herein, treatment means any manner in which the symptoms of a condition, disorder or disease or other indication, are ameliorated or otherwise beneficially altered.

As used herein, therapeutic effect means an effect resulting from treatment of a subject that alters, typically improves or ameliorates the symptoms of a disease or condition or that cures a disease or condition. A therapeutically effective amount refers to the amount of a composition, molecule or compound which results in a therapeutic effect following administration to a subject.

As used herein, the term “subject” refers to an animal, including a mammal, such as a human being.

As used herein, a patient refers to a human subject exhibiting symptoms of a disease or disorder.

As used herein, amelioration of the symptoms of a particular disease or disorder by a treatment, such as by administration of a pharmaceutical composition or other therapeutic, refers to any lessening, whether permanent or temporary, lasting or transient, of the symptoms that can be attributed to or associated with administration of the composition or therapeutic.

As used herein, prevention or prophylaxis refers to methods in which the risk of developing a disease or condition is reduced.

As used herein, a “therapeutically effective amount” or a “therapeutically effective dose” refers to the quantity of an agent, compound, material, or composition containing a compound that is at least sufficient to produce a therapeutic effect. Hence, it is the quantity necessary for preventing, curing, ameliorating, arresting or partially arresting a symptom of a disease or disorder.

As used herein, unit dose form refers to physically discrete units suitable for human and animal subjects and packaged individually as is known in the art.

As used herein, a single dosage formulation refers to a formulation containing a single dose of therapeutic agent for direct administration. Single dosage formulations generally do not contain any preservatives.

As used herein, “direct administration” refers to formulation of a composition for administration without dilution.

As used herein, a multi-dose formulation refers to a formulation that contains multiple doses of a therapeutic agent and that can be directly administered to provide several single doses of the therapeutic agent. The doses can be administered over the course of minutes, hours, weeks, days or months. Multidose formulations can allow dose adjustment, dose-pooling and/or dose-splitting. Because multi-dose formulations are used over time, they generally contain one or more preservatives to prevent microbial growth.

As used herein, “parenteral administration” refers to administration routes that achieve systemic administration. Exemplary parenteral routes of administration include, for example, intravenous, subcutaneous or intramuscular administration.

As used herein, a “collection” refers to a collection containing at least 10 different proteins and/or active portions thereof, and generally containing at least 50, 100, 500, 1000, 10⁴, 10⁵ or more members. The collections typically contain proteins to be screened for activity. Included in the collections are naturally occurring proteins (or active portions thereof) and/or modified proteins. The modifications include random mutations along the length of the protein and/or modifications in targeted or selected regions (i.e., focused mutations). The modifications can be combinatorial and can include all permutations, by substitution of all amino acids at a particular locus or at all loci or subsets thereof. The collections can include proteins of full length or shorter. The size of the collection and particular collection is determined by the user. The term collection herein is used interchangeably with the term “library” and mean the same thing.

As used herein, an “article of manufacture” is a product that is made and sold. As used throughout this application, the term is intended to encompass a therapeutic agent with a soluble PH20, such as esPH20, or an esPH20 alone, contained in the same or separate articles of packaging.

As used herein, fluid refers to any composition that can flow. Fluids thus encompass compositions that are in the form of semi-solids, pastes, solutions, aqueous mixtures, gels, lotions, creams and other such compositions.

As used herein, a “control” or “standard” refers to a sample that is substantially identical to the test sample, except that it is not treated with a test parameter, or, if it is a plasma sample, it can be from a normal volunteer not affected with the condition of interest. A control also can be an internal control. For example, a control can be a sample, such as a virus, that has a known property or activity.

As used herein, the singular forms “a,” “an” and “the” include plural referents unless the context clearly dictates otherwise. Thus, for example, reference to “an” agent includes one or more agents.

As used herein, the term “or” is used to mean “and/or” unless explicitly indicated to refer to alternatives only or the alternatives are mutually exclusive.

As used herein, ranges and amounts can be expressed as “about” a particular value or range. About also includes the exact amount. Hence “about 5 bases” means “about 5 bases” and also “5 bases.”

As used herein, “optional” or “optionally” means that the subsequently described event or circumstance does or does not occur, and that the description includes instances where said event or circumstance occurs and instances where it does not. For example, an optionally substituted group means that the group is unsubstituted or is substituted.

As used herein, the abbreviations for any protective groups, amino acids and other compounds, are, unless indicated otherwise, in accord with their common usage, recognized abbreviations, or the IUPAC-IUB Commission on Biochemical Nomenclature (see, (1972) Biochem. 11:1726).

For clarity of disclosure, and not by way of limitation, the detailed description is divided into the subsections that follow.

### B. PH20 HYALURONIDASE AND THERMAL STABILITY

PH20 hyaluronidase (also known as sperm surface protein, sperm adhesion molecule 1 or SPAM1) is a therapeutic protein that acts as a spreading agent to increase subcutaneous delivery of other co-administered agents. PH20 hyaluronidase also exhibits therapeutic activity itself to treat a number of diseases and conditions associated with accumulated hyaluronan (HA) levels, such as a variety of tumors and cancers.

PH20 exhibits its therapeutic activity by virtue of its ability to hydrolyze hyaluronan (also called hyaluronic acid, hyaluronate or HA), which is found in connective tissues such as the extracellular matrix and is a major constituent of the interstial barrier. Hyaluronan is a non-sulfated glycosaminoglycan that is widely distributed throughout connective, epithelial, and neural tissues. Hyaluronan polymers are composed of repeating disaccharide units, D-glucuronic acid (GlcA) and N-acetyl-D-glucosamine (GlcNAc), linked together via alternating β-1→4 and β-1→3 glycosidic bonds. Hyaluronan chains can reach about 25,000 disaccharide repeats or more in length, and polymers of hyaluronan can range in size from about 5,000 to 20,000,000 Da in vivo. PH20 is an endo-β-N-acetyl-hexosaminidase that hydrolyzes the β1→4 glycosidic bond of hyaluronic acid into various oligosaccharide lengths such as tetrasaccharides and hexasaccharides. PH20 has both hydrolytic and transglycosidase activities. In addition to degrading hyaluronic acid, PH20 also can degrade chondroitin sulfates, such as C4-S and C6-S. PH20 can exhibit hyaluronidase activity at acidic pH and neutral pH.

PH20 hyaluronidase, however, is susceptible to degradation and denaturation at elevated temperatures. Provided herein are modified PH20 hyaluronidase polypeptides that exhibit stability under thermal stress conditions of about or at least or greater than 52° C. for 10 minutes, and hence are designated uber-thermophiles. By virtue of the thermal stability, the modified PH20 polypeptides provided herein are tolerant to heat and exhibit improved protein thermodynamic stability to extend product shelf life. In addition, the modified PH20 polypeptides permit storage and use in a wider range of temperature conditions. For example, the modified PH20 polypeptides can be employed or stored under conditions in varied climates without refrigeration.

1. Structure

PH20 cDNA has been cloned from numerous mammalian species. Exemplary PH20 precursor polypeptides include, but are not limited to, human (SEQ ID NO:6), bovine (SEQ ID NOS:15 or 17), rabbit (SEQ ID NO:23), Cynomolgus monkey (SEQ ID NO:13), guinea pig (SEQ ID NO:28), rat (SEQ ID NO:21), mouse (SEQ ID NO:19), chimpanzee (SEQ ID NO:8, SEQ ID NO:9 or SEQ ID NO:399), Rhesus monkey (SEQ ID NO:11), fox (SEQ ID NO:30), gibbon (SEQ ID NO:387), marmoset (SEQ ID NO:389) or orangutan (SEQ ID NO:391) PH20 polypeptides. The mRNA transcript is typically translated to generate a precursor protein containing a 35 amino acid signal sequence at the N-terminus. Following transport to the ER, the signal peptide is removed to yield a mature PH20 polypeptide. Exemplary mature PH20 polypeptides include, but are not limited to, human (SEQ ID NO:7), bovine (SEQ ID NOS:16 or 18), rabbit (SEQ ID NO:24), Cynomolgus monkey (SEQ ID NO:14), guinea pig (SEQ ID NO:29), rat (SEQ ID NO:22), mouse (SEQ ID NO:20), chimpanzee (SEQ ID NO:10 or SEQ ID NO:400), Rhesus monkey (SEQ ID NO:12), fox (SEQ ID NO:31), gibbon (SEQ ID NO:388), marmoset (SEQ ID NO:390) or orangutan (SEQ ID NO:392) PH20 polypeptides. For example, the human PH20 mRNA transcript is normally translated to generate a 509 amino acid precursor protein (SEQ ID NO:6) containing a 35 amino acid signal sequence at the N-terminus (amino acid residue positions 1-35 of SEQ ID NO:6). Thus, following transport to the ER and removal of the signal peptide, a 474 amino acid mature polypeptide with an amino acid sequence set forth in SEQ ID NO:7 is produced. Sequences of PH20 from ovine are also known (see e.g., SEQ ID NOS: 25-27).

In particular, human PH20 has the sequence of amino acids set forth in SEQ ID NO:6. The mature human PH20 lacking a signal sequence is set forth in SEQ ID NO:7. Allelic variants and other variants of PH20 are known. Other sequences of PH20 have been reported. For example, a PH20 variant is known as set forth in the precursor sequence set forth in SEQ ID NO:68 that contains an Ala at position 48 and a Trp at position 499, or the mature sequence thereof set forth in SEQ ID NO:69 containing the corresponding differences at positions 13 and 464, respectively, compared to the sequence set forth in SEQ ID NO:7 (see e.g., Gmachl et al. (1993) FEBS Lett., 336:545-548; GenBank Accession No. AAC60607). Further, a natural variant of PH20 has been identified containing a Glutamine (Gln; Q) at position 5 as compared to the precursor sequence of amino acids set forth in SEQ ID NO:6 (see e.g., SEQ ID NO:70, see also Varela et al. (2011) Nature, 469:539-542). Another natural variant contains an Alanine (Ala; A) at position 47 compared to the sequence of amino acids set forth in SEQ ID NO:6 (as set forth in SEQ ID NO: 71) and corresponding to position 12 compared to the sequence of amino acids set forth in SEQ ID NO: 3 or 7 (as set forth in SEQ ID NO:72).

The sequence and structure of PH20 polypeptides are highly conserved. Sequence identity between and among PH20 proteins from various species is about 50% to 90%. The hydrophobic N-terminal signal sequence of 35 amino acids in length is generally conserved among PH20 hyaluronidase polypeptides. PH20 hyaluronidases contain a common core hyaluronidase domain region of about 340 amino acids in length that corresponds to amino acid residues 38-374 of the precursor human PH20 sequence set forth in SEQ ID NO:6. A mature PH20 polypeptide lacking the signal sequence and containing a contiguous sequence of amino acids having a C-terminal amino acid residue corresponding to amino acid residue 464 of SEQ ID NO:6 (e.g., amino acid residues corresponding to positions 36-464 of the amino acid sequence set forth in SEQ ID NO:6) is the minimal sequence required for hyaluronidase activity (see e.g., U.S. patent application Ser. No. 10/795,095, which is issued as U.S. Pat. No. 7,767,429; see also U.S. Publication No. US20100143457).

Within the common hyaluronidase domain region, at least 57 amino acids are conserved between and among species (see e.g., Arming et al. (1997) Eur. J. Biochem., 247:810-814; ten Have et al. (1998) Reprod. Fertil. Dev., 10:165-72; Chowpongpang et al. (2004) Biotechnology Letters, 26:1247-1252). For example, PH20 hyaluronidases contain 12 conserved cysteine residues corresponding to amino acid residue 25, 189, 203, 316, 341, 346, 352, 400, 402, 408, 423 and 429 of the sequence of amino acids of a mature PH20 lacking the signal sequence such as set forth in SEQ ID NO: 7 or set forth in SEQ ID NO:3 or other soluble C-terminal truncated polypeptides (corresponding to amino acid residues 60, 224, 238, 351, 376, 381, 387, 435, 437, 443, 458 and 464 of full-length human PH20 set forth in SEQ ID NO:6). Cysteine residues corresponding to 25 and 316 and cysteine residues corresponding to 189 and 203 form disulfide bridges. The other cysteine residues also form disulfide bridges, are involved in posttranslational protein maturation and/or in activity modulation. For example, further four disulfide bonds are formed between the cysteine residues C376 and C387; between C381 and C435; between C437 and C443; and between C458 and C464 of the polypeptide exemplified in SEQ ID NO:6 (corresponding to positions C341 and C352; between C346 and C400; between C402 and C408; and between C423 and C429, respectively, of the mature polypeptide set forth in SEQ ID NO:3 or 7).

Amino acid residues corresponding to amino acid residue D111, E113 and E249 of the sequence of amino acids set forth in SEQ ID NO: 3 or 7 (or other mature soluble C-terminally truncated PH20 polypeptides) are acidic residues in the enzyme active site and are conserved between and among PH20 species Amino acid residues corresponding to amino acid residues R176, 8246, 8252 of the sequence of amino acids set forth in SEQ ID NO: 3 or 7 (or other mature soluble C-terminally truncated PH20 polypeptides) are also conserved between and among species and contribute to substrate binding and/or hyaluronidase activity. Amino acid mutations D111N, E113Q, R176G, E249N and R252T result in enzymes that have no detectable enzymatic activity or residual enzymatic activity (see e.g., Arming et al. (1997) Eur. J. Biochem., 247:810-814).

The Examples herein confirm the requirement of PH20 amino acid residues corresponding to positions 25, 111, 113, 176, 189, 203, 246, 249, 252, 316, 341, 346, 352, 400, 402, 408, 423 and 429 of the sequence of amino acids set forth in a mature PH20 lacking the signal sequence such as set forth in SEQ ID NO: 3 or 7 for hyaluronidase activity, since mutagenesis of these residues results in an enzyme that is not active (e.g., it is not expressed or is inactive when expressed, see e.g., Table 8). The exception is that amino acid replacement corresponding to R176K and C316D resulted in mutants that generated some residual hyaluronidase activity.

Glycosylation also is required for PH20 hyaluronidase activity based on the recognition motif NxS or NxT. There are six N-linked oligosaccharides at amino acid residues corresponding to positions N47, N131, N200, N219, N333 and N358 of the mature sequence of amino acids set forth in SEQ ID NO: 7 or SEQ ID NO:3 or other soluble C-terminally truncated polypeptide (corresponding to amino acid residues N82, N166, N235, N254, N368 and N393 of human PH20 set forth in SEQ ID NO: 6). In particular, at least N-linked glycosylation sites corresponding to amino acid residues N200, N333 and N358 are required for secretion and/or activity of the enzyme (see e.g., U.S. Publication No. US20100143457). For example, a PH20 polypeptide containing amino acid mutations N200A, N333A, N358A or N333A/N393A result in inactive proteins. Single mutations of glycosylation sites N47A, N131A, N219A, and double mutations of glycosylation sites N47A/N131A, N47A/N219A, N131A/N291A retain activity. The N-linked glycosylation site corresponding to amino acid residue N368 of human PH20 set forth in SEQ ID NO:6 is conserved between and among species (see e.g., Chowpongpang et al. (2004) Biotechnology Letters, 26:1247-1252). PH20 hyaluronidases also contains O-linked glycosylation sites. For example, human PH20 has one O-linked oligosaccharide at the amino acid residue corresponding to amino acid T440 of the sequence of amino acids set forth in SEQ ID NO:3 or 7 (corresponding to amino acid residue T475 in SEQ ID NO:6).

In addition to the catalytic sites, PH20 also contains a hyaluronan-binding site. This site is located in the Peptide 2 region, which corresponds to amino acid positions 205-235 of the precursor polypeptide set forth in SEQ ID NO:6 and positions 170-200 of the mature polypeptide set forth in SEQ ID NO:3 or 7. This region is highly conserved among hyaluronidases and is similar to the heparin binding motif Mutation of the arginine residue at position 176 (corresponding to the mature PH20 polypeptide set forth in SEQ ID NO:3 or 7) to a glycine results in a polypeptide with only about 1% of the hyaluronidase activity of the wild type polypeptide (Arming et al., (1997) Eur. J. Biochem. 247:810-814).

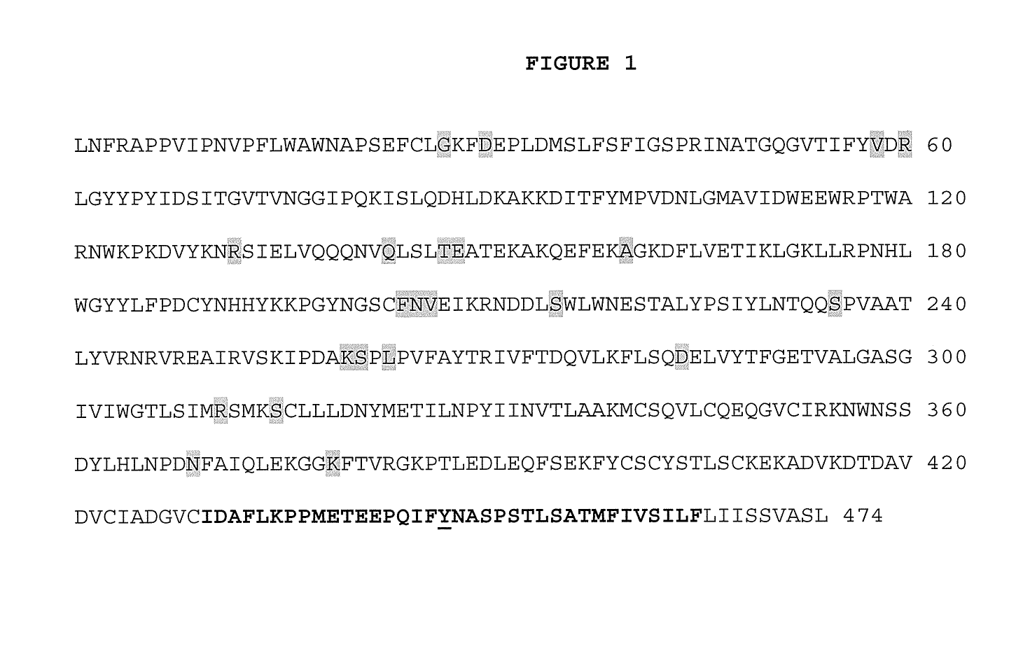

PH20 polypeptides contain a glycosyl phosphatidylinositol (GPI) anchor attached to the C-terminus of the protein that anchors the protein to the extracellular leaflet of the plasma membrane of cells. At least human, monkey, mouse and guinea pig PH20 are strongly attached to the plasma membrane via the GPI anchor, which can be released by treating with phosphatidylinositol-specific phospholipase C (PI-PLC; see e.g., Lin et al. (1994) Journal of Cell Biology, 125:1157-1163; Lin et al. (1993) Proc. Natl. Acad. Sci., 90:10071-10075). Other PH20 enzymes, such as bovine PH20, are loosely attached to the plasma membrane and are not anchored via a phospholipase sensitive anchor. As discussed below, soluble active forms that, when expressed, are not attached to the membrane but are secreted can be generated by removal of all of a portion of the GPI anchor attachment signal site (see also U.S. Pat. No. 7,767,429; U.S. Publication No. US20100143457). These include, for example, soluble PH20 polypeptides set forth in any of SEQ ID NOS: 3 or 32-66, or precursor forms thereof containing a signal sequence. It is understood herein that reference to positions herein above in a mature PH20 polypeptide set forth in SEQ ID NO:3 or 7 are the same positions found in the C-terminally truncated polypeptides set forth in SEQ ID NOS:32-66 (see FIG. 1).

GPI-anchored proteins, for example human PH20, are translated with a cleavable N-terminal signal peptide that directs the protein to the endoplasmic reticulum (ER). At the C-terminus of these proteins is another signal sequence that directs addition of a preformed GPI-anchor to the polypeptide within the lumen of the ER. Addition of the GPI anchor occurs following cleavage of the C-terminal portion at a specific amino acid position, called the ω-site (typically located approximately 20-30 amino acids from the C-terminus). Although there appears to be no consensus sequence to identify the location of the ω-site, GPI anchored proteins contain a C-terminal GPI-anchor attachment signal sequence or domain that typically contains a predominantly hydrophobic region of 8-20 amino acids, preceded by a hydrophilic spacer region of 8-12 amino acids immediately downstream of the ω-site. This hydrophilic spacer region often is rich in charged amino acids and proline (White et al. (2000) J. Cell Sci. 113(Pt.4):721-727). There is generally a region of approximately 11 amino acids before the ω−1 position that is characterized by a low amount of predicted secondary structure, a region around the cleavage site (ω-site), from ω−1 to ω+2 that is characterized by the presence of small side chain residues, the spacer region between positions ω+3 and ω+9, and a hydrophobic tail from ω+10 to the C-terminal end (Pierleoni et al., (2008) BMC Bioinformatics 9:392).

Although there is no GPI-anchor attachment signal consensus sequence, various in silico methods and algorithms have been developed that can be used to identify such sequences in polypeptides (see, e.g., Udenfriend et al. (1995) Methods Enzymol. 250:571-582; Eisenhaber et al. (1999) J. Mol. Chem. 292: 741-758; Kronegg and Buloz, (1999), “Detection/prediction of GPI cleavage site (GPI-anchor) in a protein (DGPI),” 129.194.185.165/dgpi/; Fankhauser et al. (2005) Bioinformatics 21:1846-1852; Omaetxebarria et al. (2007) Proteomics 7:1951-1960; Pierleoni et al. (2008) BMC Bioinformatics 9:392), including those that are readily available on bioinformatic websites, such as the ExPASy Proteomics tools site (expasy.ch/tools/). Thus, one of skill in the art can determine whether a PH20 polypeptide likely contains a GPI-anchor attachment signal sequence, and, therefore, whether the PH20 polypeptide is a GPI-anchored protein.

The covalent attachment of a GPI-anchor to the C-terminus of human PH20 and, therefore, the membrane-bound nature of PH20, has been confirmed using phosphatidylinositol-specific phospholipase C (PI-PLC) hydrolysis studies (see e.g., Lin et al., (1994) J. Biol. Chem. 125:1157-1163). Phosphatidylinositol-specific phospholipase C (PI-PLC) and D (PI-PLD) hydrolyze the GPI anchor, releasing the PH20 polypeptide from the cell membrane. The prior art literature reports that a ω-site cleavage site of human PH20 is identified between Ser490 and Ala491 and for monkey PH20 is identified between Ser491 and Thr492 (Lin et al. (1993) Proc. Natl. Acad. Sci, (1993) 90:10071-10075). Thus, the literature reports that a GPI-anchor attachment signal sequence of human PH20 is located at amino acid positions 491-509 of the precursor polypeptide set forth in SEQ ID NO:6, and the w-site is amino acid position 490. Thus, in this modeling of human PH20, amino acids 491-509 are cleaved following transport to the ER and a GPI anchor is covalently attached to the serine residue at position 490.

Soluble PH20 Polypeptides

PH20 can exist in membrane-bound or membrane-associated form, or can be secreted into the media when expressed from cells, and thereby can exist in soluble form. Soluble PH20 can be detected and discriminated from insoluble, membrane-bound PH20 using methods well known in the art, including, but not limited to, those using a Triton® X-114 assay. In this assay, soluble PH20 hyaluronidases partition into the aqueous phase of a Triton® X-114 solution warmed to 37° C. (Bordier et al., (1981) J. Biol. Chem., 256:1604-7) while membrane-anchored PH20 hyaluronidases partition into the detergent rich phase. Thus, in addition to using algorithms to assess whether a PH20 polypeptide is naturally GPI-anchored and hence membrane-bound, solubility experiments also can be performed.

Soluble PH20 enzymes include hyaluronidases that contain a GPI-anchor attachment signal sequence, but that are loosely attached to the membrane such that they do not contain a phospholipase sensitive anchor. For example, soluble PH20 polypeptides include ovine or bovine PH20. Various forms of such soluble PH20 hyaluronidases have been prepared and approved for therapeutic use in subjects, including humans. For example, animal-derived hyaluronidase preparations include Vitrase® (ISTA Pharmaceuticals), a purified ovine testicular hyaluronidase, and Amphadase® (Amphastar Pharmaceuticals), a bovine testicular hyaluronidase. Soluble PH20 enzymes also include C-terminal truncated forms of non-human or human membrane-associated PH20 hyaluronidases that lack one or more amino acid residues of a glycosylphosphatidylinositol (GPI) anchor attachment signal sequence and that retain hyaluronidase activity (see e.g., U.S. Pat. No. 7,767,429; U.S. Publication No. US20100143457). Thus, instead of having a GPI-anchor covalently attached to the C-terminus of the protein in the ER and being anchored to the extracellular leaflet of the plasma membrane, these polypeptides are secreted when expressed from cells and are soluble. In instances where the soluble PH20 retains a portion of the GPI anchor attachment signal sequence, 1, 2, 3, 4, 5, 6, 7, 8, 9, 10 or more amino acid residues in the GPI-anchor attachment signal sequence can be retained, provided the polypeptide is soluble (i.e., secreted when expressed from cells) and active.

Exemplary soluble hyaluronidases that are C-terminally truncated and lack all or a portion of the GPI anchor attachment signal sequence include, but are not limited to, PH20 polypeptides of primate origin, such as, for example, human and chimpanzee PH20 polypeptides. For example, soluble PH20 polypeptides can be made by C-terminal truncation of a polypeptide set forth in SEQ ID NOS:7, 10, 12, 14, 69, 72, 388, 390, 392 or 400 or variants thereof that exhibit at least 80%, 85%, 90%, 95% or more sequence identity to any of SEQ ID NO: 7, 10, 12, 14, 69, 72, 388, 390, 392 or 400, wherein the resulting polypeptide is active, soluble and lacks all or a portion of amino acid residues from the GPI-anchor attachment signal sequence.

Exemplary soluble PH20 polypeptides are C-terminal truncated human PH20 polypeptides that are mature (lacking a signal sequence), soluble and exhibit neutral activity, and that contain a contiguous sequence of amino acids set forth in SEQ ID NO:6 or SEQ ID NO:7 that minimally has a C-terminal truncated amino acid residue at or after amino acid residue 464 of the sequence of amino acids set forth in SEQ ID NO:6. For example, soluble PH20 polypeptides include C-terminal truncated polypeptides that minimally contain a contiguous sequence of amino acids 36-464 of SEQ ID NO:6, or includes a sequence of amino acids that has at least 85%, for example at least 86%, 87%, 88%, 89%, 90%, 91%, 92%, 93%, 94%, 95%, 96%, 97%, 98% sequence identity to a contiguous sequence of amino acids that has a C-terminal amino acid residue after amino acid 464 of SEQ ID NO:6 and retains hyaluronidase activity.

Exemplary C-terminally truncated human PH20 polypeptides are mature polypeptides (lacking a signal sequence) that include a contiguous sequence of amino acids set forth in SEQ ID NO:6 with a C-terminal residue after 464 such as after amino acid position 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499 or 500 of the sequence of amino acids set forth in SEQ ID NO:6, or a variant thereof that exhibits at least 85% sequence identity, such as at least 86%, 87%, 88%, 89%, 90%, 91%, 92%, 93%, 94%, 95%, 96%, 97%, 98% sequence identity thereto and retains hyaluronidase activity. For example, exemplary C-terminal PH20 polypeptides have a sequence of amino acids 36 to 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499 or 500 of the sequence of amino acids set forth in SEQ ID NO:6, or a variant thereof that exhibits at least 85% sequence identity, such as at least 86%, 87%, 88%, 89%, 90%, 91%, 92%, 93%, 94%, 95%, 96%, 97%, 98% sequence identity thereto and retains hyaluronidase activity. Soluble PH20 polypeptides include any that has the sequence of amino acids set forth in SEQ ID NOS: 3 or 32-66 or a sequence of amino acids that exhibits at least 85% sequence identity, such as at least 86%, 87%, 88%, 89%, 90%, 91%, 92%, 93%, 94%, 95%, 96%, 97%, 98% sequence identity to the sequence of amino acids set forth in any of SEQ ID NOS: 3 or 32-66.

In particular, a soluble human PH20 polypeptide is a polypeptide that is truncated after amino acid 482 of the sequence set forth in SEQ ID NO:6. Such a polypeptide can be generated from a nucleic acid molecule containing a signal sequence and encoding amino acids 36-482, for example, as set forth in SEQ ID NO:1 (containing an IgG kappa signal sequence) or SEQ ID NO:67 (containing the native signal sequence). Post translational processing removes the signal sequence, leaving a 447 amino acid soluble recombinant human PH20 (SEQ ID NO:3). A product produced upon expression of a vector set forth in SEQ ID NO:4 or 5, and containing a nucleic acid molecule set forth in SEQ ID NO:67, results in a secreted product, designated rHuPH20, in the culture medium that exhibits heterogeneity at the C-terminus such that the product includes a mixture of species that can include any one or more of SEQ ID NOS: 3 and 44-48 in various abundance. Typically, rHuPH20 is produced in cells that facilitate correct N-glycosylation to retain activity, such as mammalian cells, for example CHO cells (e.g., DG44 CHO cells). Hylenex® (Halozyme) is a human recombinant hyaluronidase produced by genetically engineered Chinese Hamster Ovary (CHO) cells containing nucleic acid encoding a truncated human PH20 polypeptide (designated rHuPH20).

2. Function

PH20 is normally expressed in sperm from a single testis-specific gene. PH20 is a sperm-associated protein involved in fertilization. PH20 is normally localized on the sperm surface, and in the lysosome-derived acrosome, where it is bound to the inner acrosomal membrane. PH20 is multifunctional and exhibits hyaluronidase activity, hyaluronan (HA)-mediated cell-signaling activity, and acts as a sperm receptor for the zona pellucida surrounding the oocyte when present on acrosome reacted (AR) sperm. For example, PH20 is naturally involved in sperm-egg adhesion and aids penetration by sperm of the layer of cumulus cells by digesting hyaluronic acid. In addition to being a hyaluronidase, PH20 also appears to be a receptor for HA-induced cell signaling, and a receptor for the zona pellucida surrounding the oocyte. Due to the role of PH20 in fertilization, PH20 can be used as an antigen for immunocontraception.

PH20 is a neutral active hyaluronidase, although it can exhibit acid-active activity in some cases. The hyaluronidase activity of PH20 is exhibited by the plasma membrane- and inner acrosomal membrane-associated PH20. The plasma membrane PH20 exhibits hyaluronidase activity only at neutral pH, while the inner acrosomal membrane-associated PH20 exhibits acid-active enzyme activity. The structural basis for these differences is due to the presence of two catalytic sites in PH20. A first catalytic site is designated the Peptide 1 region, corresponding to amino acid residues 142-172 of SEQ ID NO:6, which is involved in enzyme activity of PH20 at neutral pH. A second catalytic site is designated the peptide 3 region, corresponding to amino acid residues 277-297 of SEQ ID NO:6, which is involved in enzyme activity at lower pH. A change in the structure of the inner acrosomal membrane-associated PH20 occurs after the acrosome reaction, whereby PH20 is endoproteolytically cleaved but held together by disulfide bonds. The result of the endoproteolysis is that the peptide 3 region is activated and can thus effect neutral and acid-activity to PH20 (see e.g., Cherr et al. (2001) Matrix Biology, 20:515-525. Also, after the acrosome reaction, lower molecular weight forms are generated by release from the inner acrosomal membrane (e.g., a 53 kDa soluble form of PH20 is generated in monkey). The lower molecular weight form(s) also is acid active.

The hyaluronidase activity of PH20 accounts for the spreading activity observed in animal testes extracts that have been used clinically for decades to increase the dispersion and absorption of drugs (see e.g., Bookbinder et al. (2006) J Controlled Release, 114:230-241). For example, pharmaceutical preparations containing hyaluronidase were developed as fractionated extracts from bovine testes for therapeutic use as spreading agents and in other applications (Schwartzman (1951) J. Pediat., 39:491-502). Original bovine testicular extract preparations included, for example, extracts sold under the trademarks Wydase®, Hylase®, “Dessau,” Neopermease®, Alidase® and Hyazyme®. It is now known that the spreading activity of testicular extract preparations are due to PH20 hyaluronidase activity. For example, in 2001 a sperm hyaluronidase in bull was identified as the hyaluronidase PH20 (Lalancette et al. (2001) Biol. Reprod., 65:628-36). By catalyzing the hydrolysis of hyaluronic acid, PH20 hyaluronidase lowers the viscosity of hyaluronic acid, thereby increasing tissue permeability. Hence, soluble forms of PH20 are used as a spreading or dispersing agent in conjunction with other agents, drug and proteins to enhance their dispersion and delivery, and to improve the pharmacokinetic and pharmacodynamic profile of the coadministered agent, drug or protein (see e.g., U.S. Pat. No. 7,767,429; Bookbinder et al. (2006) J Controlled Release, 114:230-241).

3. Thermal Stability of PH20 Hyaluronidases

PH20 hyaluronidase is not stable at elevated temperatures. As shown in the Examples herein, the Tₘ, of the exemplary soluble PH20 designated rHuPH20 is about 44° C. (see e.g. Example 5). Also, hyaluronidase activity is reduced by about 50% or more upon exposure to temperatures greater than 49° C. for only 10 minutes, with less than 20% activity retained upon exposure to temperatures of 55° C. or higher for only 10 minutes (see Example 6). The temperature profile of PH20 hyaluronidase demonstrates that it is susceptible to denaturation by small increases in temperature.

The thermal instability of PH20 hyaluronidase can be a problem in developing formulations of PH20 that require storage at high temperatures and/or are otherwise exposed to high temperatures during storage or use (e.g. greater than room temperature or ambient temperatures, such as greater than 30° C., 35° C., 40° C., 45° C. or greater). In particular, temperatures can fluctuate under field conditions in which the therapeutic protein is exposed, such as conditions associated with storage, transport, handling and delivery. These environmental changes are generally not possible to control. For example, refrigeration or temperature control is not always available to the end user of the therapeutic protein, thereby requiring the protein to be stored without refrigeration for prolonged periods of time. This is particularly a concern in areas that experience tropical climates. In addition, routes of administration and certain administration devices also can expose a protein to high temperatures, including fluctuating temperatures. For example, pumps, implantable devices, depot injections and other sustained delivery of proteins can require that a formulation is stable at elevated temperatures of 37° C. or higher over the operational life of the device.

Although stability of formulations containing PH20 hyaluronidase, or other hyaluronan-degrading enzymes, can be achieved by a variety of stabilizing substances, such substances can adversely affect downstream use of the stored protein or other co-formulated proteins. For example, stabilizing agents (e.g. surfactants and other stabilizing agents) can decrease long term hyaluronidase activity, increase aggregation, increase denaturation and/or promote oxidation. In formulations of PH20 hyaluronidase that are co-formulated with other agents, stabilizing agents also can similarly destabilize the activity, absorption or aggregation of the other agent. These effects can be exacerbated at elevated or fluctuating temperatures. This means that in some cases PH20 hyaluronidase formulations cannot be stored for long term or under high or fluctuating temperature conditions even with a stabilizing agent. In some cases, storage of PH20 hyaluronidase with a stabilizer can necessitate the removal of one or more stabilizing substances before the protein can be used in a downstream process or co-formulated with other agents.

As a therapeutic agent, however, it is desirable to generate formulations of PH20 hyaluronidase to store for later use or for sustained delivery. It is important that the protein is stored under conditions that preserve the stability of the protein under various conditions including temperature. The modified PH20 polypeptides provided herein are uber-thermophiles that are tolerant to temperatures in which the unmodified PH20 polypeptide is not stable. The following sections describe in further detail uber-thermophile PH20 polypeptides provided herein. Also described below are compositions, combinations, methods and applications of the PH20 uber-thermophile polypeptides.

### C. MODIFIED PH20 POLYPEPTIDES: UBER-THERMOPHILES

Provided herein are modified or variant PH20 polypeptides that are uber-thermophiles. These uber thermophiles exhibit increased thermostability compared to the unmodified PH20 polypeptide not containing the modification (e.g. a wildtype PH20, such as a full-length mature PH20 or soluble C-terminal truncated fragment thereof). The modified PH20 polypeptides provided herein that are uber thermophiles retain at least 50% of their hyaluronidase activity after incubation at 52 C for 10 minutes compared to the hyaluronidase activity after incubation at 4° C. for 10 minutes. Activity is assessed on a substrate for the unmodified hyaluronidase. For example, among the modified PH20 polypeptides provided herein are polypeptides that retain at least 55%, 60%, 65%, 70%, 75%, 80%, 85%, 90%, 95% or greater of its hyaluronidase activity after incubation at 52° C. for 10 minutes compared to its hyaluronidase activity after incubation at 4° C. for 10 minutes.

Hence, the modified PH20 polypeptides can be used under conditions that require storage at high temperatures and/or are otherwise exposed to high temperatures during storage or use (e.g. greater than room temperature or ambient temperature, such as greater than 25° C., 30° C., 35° C., 37° C., 40° C., 45° C. or greater). For example, any of the modified PH20 polypeptides provided herein can be stored without refrigeration, including under ambient conditions where temperatures fluctuate (e.g. during transport, delivery or handling) or under tropical climate conditions. In another example, any of the modified PH20 polypeptides provided herein are suitable for use in sustained delivery methods requiring exposure to elevated temperatures greater than 25° C., and typically greater than 30° C., 35° C., 37° C. or higher over the course of use. In any such examples, any of the modified PH20 polypeptides provided herein can exhibit stability (e.g. retain greater than 50% hyaluronidase activity) achieved by exposure to non-refrigerated or ambient temperatures (e.g. greater than 25° C., such as in a range that is 30° C. to 42° C., inclusive, such as at least 30° C. or 37° C. or higher) for at least 72 hours, 96 hours, days, one week, two weeks, three weeks, one month, two months, three months, four months, five months, six months or more.

The modified PH20 uber-thermophile polypeptides provided herein contain one or more than one modification in an unmodified PH20 polypeptide not containing the modification (e.g. a wildtype PH20, such as a full-length mature PH20 or soluble C-terminal truncated fragment thereof). The modifications can be a single amino acid modification, such as single amino acid replacements (substitutions), insertions or deletions, or multiple amino acid modifications, such as multiple amino acid replacements, insertions or deletions. Exemplary modifications are amino acid replacements, including single or multiple amino acid replacements. The amino acid replacement can be a conservative substitution, such as set forth in Table 2, or a non-conservative substitution, such as any described herein. Modified PH20 polypeptides provided herein can contain at least or 1, 2, 3, 4, 5, 6, 7, 8, 9, 10, 11, 12, 13, 14, 15, 16, 17, 18, 19, 20, or more modified positions compared to the PH20 polypeptide not containing the modification(s). It is understood that in any of such examples, the modified PH20 polypeptide is one that retains at least 50% of its hyaluronidase activity after incubation at 52° C. for 10 minutes compared to its hyaluronidase activity after incubation at 4° C. for 10 minutes.

The modifications described herein can be in any PH20 polypeptide (i e unmodified PH20), including precursor, mature, or C-terminal truncated forms, so long as the modified form exhibits hyaluronidase activity and retains at least 50% of its hyaluronidase activity after incubation at 52° C. for 10 minutes compared to its hyaluronidase activity after incubation at 4° C. for 10 minutes.

For example, the PH20 polypeptides contain modifications compared to a wildtype, native or reference PH20 polypeptide set forth in any of SEQ ID NOS: 2, 3, 6-66, 68-72, 387-392, 399 or 400, or in a polypeptide that has a sequence of amino acids that is at least 65%, 70%, 75%, 80%, 85%, 86%, 87%, 88%, 89%, 90%, 91%, 92%, 93%, 94%, 95%, 96%, 97%, 98%, 99% identical to any of SEQ ID NOS: 3, 6-66, 68-72, 387-392, 399 or 400. For example, the modifications are made in a human PH20 polypeptide having the sequence of amino acids including or set forth in SEQ ID NO:7, SEQ ID NO:69 or SEQ ID NO:72; a bovine PH20 polypeptide having a sequence of amino acids including or set forth in SEQ ID NOS:16 or 18; a rabbit PH20 polypeptide having a sequence of amino acids including or set forth in SEQ ID NO:24; a Cynomolgus monkey PH20 polypeptide having a sequence of amino acids including or set forth in SEQ ID NO:14; a guinea pig PH20 polypeptide having a sequence of amino acids including or set forth in SEQ ID NO:29; a rat PH20 polypeptide having a sequence of amino acids including or set forth in SEQ ID NO:22; a mouse PH20 polypeptide having a sequence of amino acids including or set forth in SEQ ID NO:20; a chimpanzee PH20 polypeptide having a sequence of amino acids including or set forth in SEQ ID NO:10 or 400; a Rhesus monkey PH20 polypeptide having a sequence of amino acids including or set forth in SEQ ID NO:12; a Fox PH20 polypeptide having a sequence of amino acids including or set forth in SEQ ID NO:31; a Gibbon PH20 polypeptide having a sequence of amino acids including or set forth in SEQ ID NO:388; a Marmoset PH20 polypeptide having a sequence of amino acids including or set forth in SEQ ID NO: 390; an Orangutan PH20 polypeptide having a sequence of amino acids including or set forth in SEQ ID NO:392; or a sheep PH20 polypeptide having a sequence of amino acids including or set forth in any of SEQ ID NOS: 25-27; or in sequence variants or truncated variants that exhibit at least 65%, 70%, 75%, 80%, 85%, 86%, 87%, 88%, 89%, 90%, 91%, 92%, 93%, 94%, 95%, 96%, 97%, 98%, 99% or more sequence identity to any of SEQ ID NOS: 7, 10, 12, 14, 16, 18, 20, 22, 24-27, 29, 31, 69, 72, 388, 390, 392 or 400.

In particular, provided herein are modified soluble PH20 polypeptides that are PH20 polypeptides containing a modification (e.g. amino acid replacement) provided herein, and that when expressed from cells are secreted into the media as a soluble protein. For example, the modifications are made in a soluble PH20 polypeptide that is C-terminally truncated within or near the C-terminus portion containing the GPI-anchor signal sequence of a PH20 polypeptide that contains a GPI-anchor signal sequence. The C-terminal truncation can be a truncation or deletion of 8 contiguous amino acids at the C-terminus, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more amino acids at the C-terminus, so long as the resulting C-terminally truncated polypeptide exhibits hyaluronidase activity and is secreted from cells (e.g., into the media) when expressed. In some examples, the modifications provided herein are made in a soluble PH20 polypeptide that is a C-terminally truncated polypeptide of SEQ ID NO:7, 10, 12, 14, 69, 72, 388, 390, 392 or 400 or a variant thereof that exhibits at least 70%, 75%, 80%, 85%, 86%, 87%, 88%, 89%, 90%, 91%, 92%, 93%, 94%, 95%, 96%, 97%, 98%, 99% or more sequence identity to any of SEQ ID NOS: 7, 10, 12, 14, 69, 72, 388, 390, 392 or 400.

In particular, provided herein are PH20 polypeptides that contain modifications (e.g. amino acid replacements) in a human PH20 polypeptide set forth in SEQ ID NO:7, or soluble C-terminal fragment thereof, or a polypeptide that has a sequence of amino acids that is at least 68%, 70%, 75%, 80%, 85%, 86%, 87%, 88%, 89%, 90%, 91%, 92%, 93%, 94%, 95%, 96%, 97%, 98%, 99% identical to any of SEQ ID NO:7 or a soluble C-terminal fragment thereof. For example, the modifications provided herein are made in a soluble or C-terminally truncated human PH20 polypeptide having the sequence of amino acids set forth in SEQ ID NOS: 3 or 32-66 or a sequence of amino acids that exhibits at least 70%, 75%, 80%, 85%, 86%, 87%, 88%, 89%, 90%, 91%, 92%, 93%, 94%, 95%, 96%, 97%, 98% sequence identity to the sequence of amino acids set forth in any of SEQ ID NOS: 3 or 32-66. For example, modified PH20 polypeptides provided herein contain amino acid replacements or substitutions, additions or deletions, truncations or combinations thereof with reference to the PH20 polypeptide set forth in SEQ ID NO:3. Modifications also can be made in the corresponding precursor form containing a signal peptide of any of SEQ ID NOS: 3, 7, 10, 12, 14, 16, 18, 20, 22, 24-27, 29, 31, 32-66, 69, 72, 388, 390, 392 or 400. For example, modifications provided herein can be made in a precursor form set forth in any of SEQ ID NOS: 2, 6, 8, 9, 11, 13, 15, 17, 19, 21, 23, 28, 30, 387, 389, 391 or 399 or in a variant thereof that exhibits at least 70%, 75%, 80%, 85%, 86%, 87%, 88%, 89%, 90%, 91%, 92%, 93%, 94%, 95%, 96%, 97%, 98%, 99% or more sequence identity to any of SEQ ID NOS: 2, 6, 8, 9, 11, 13, 15, 17, 19, 21, 23, 28, 30, 387, 389, 391 or 399.

The modified PH20 polypeptides provided herein exhibits at least 85%, 86%, 87%, 88%, 89%, 90%, 91%, 92%, 93%, 94%, 95%, 96%, 97%, 98%, 99% or more sequence identity compared to the unmodified PH20 polypeptide not containing the modification(s), such as an unmodified PH20 polypeptide set forth in any of SEQ ID NOS: 3-66, 68-72, 387-392, 399 or 400. In particular, modified PH20 polypeptides provided herein exhibit at least 85%, 86%, 87%, 88%, 89%, 90%, 91%, 92%, 93%, 94%, 95%, 96%, 97%, 98%, 99% or more sequence identity to a soluble C-terminal truncated human PH20 polypeptide set forth in any of SEQ ID NOS: 3 or 32-66. In examples of modified PH20 polypeptides provided herein, the modified PH20 polypeptide does not contain the sequence of amino acids set forth in any of SEQ ID NOS: 3-66, 68-72, 387-392, 399 or 400. Typically, the modified PH20 polypeptide is modified compared to a human PH20 polypeptide, and does not contain the sequence of amino acids set forth in any of SEQ ID NOS: 8-31, 69, 72, 387-392, 399 or 400.

Generally, any modification, such as amino acid replacement, deletion or substitution, can be made in a PH20 polypeptide, with the proviso that the modification is not an amino acid replacement where the only modification is a single amino acid replacement that is V12A, N47A, D111N, E113Q, N131A, R176G, N200A, N219A, E249Q, R252T, N333A or N358A. Also, where the modified PH20 polypeptide contains only two amino acid replacements, the amino acid replacements are not P13A/L464W, N47A/N131A, N47A/N219A, N131A/N219A or N333A/N358A. In a further example, where the modified PH20 polypeptide contains only three amino acid replacements, the amino acid replacements are not N47A/N131A/N219A. Exemplary modifications provided herein are described in detail below.

Typically, the modified PH20 polypeptide exhibits at least 40%, 50%, 60%, 65%, 70%, 75%, 80%, 85%, 90%, 95% or more of the hyaluronidase activity of the unmodified PH20 polypeptide not containing the modification (e.g. a wildtype PH20, such as a full-length mature PH20 or soluble C-terminal truncated fragment thereof) as assessed in a standard hyaluronidase activity assay. Such assays are described in Section G (see also Example 3 and Example 4). It is understood that a standard hyaluronidase assay is performed under conditions and temperatures in which the unmodified PH20 polypeptide is tolerant, such that the polypeptide is not incubated under conditions that result in thermal instability of the polypeptide (e.g. incubation at 52° C. for 10 minutes).

To retain hyaluronidase activity, modifications typically are not made at those positions that are less tolerant to change or required for hyaluronidase activity. For example, generally modifications are not made at a position corresponding to position 7, 16, 17, 18, 19, 21, 25, 53, 55, 56, 57, 62, 64, 76, 78, 80, 88, 95, 100, 101, 109, 111, 112, 113, 115, 116, 121, 123, 126, 129, 185, 187, 188, 189, 190, 191, 194, 199, 201, 203, 207, 210, 223, 225, 227, 228, 229, 241, 243, 244, 246, 249, 250, 252, 254, 262, 268, 295, 296, 299, 303, 319, 322, 329, 330, 332, 333, 336, 337, 340, 341, 344, 345, 346, 350, 352, 354, 355, 362, 363, 364, 365, 366, 370, 372, 382, 384, 386, 390, 400, 402, 408, 423, 424, 429 or 430, with reference to amino acid positions of the sequence set forth in SEQ ID NO:7 or 3 or other soluble C-terminal truncated fragment. Also, in examples where modifications are made at any of positions 2, 3, 4, 5, 6, 8, 9, 10, 11, 12, 13, 14, 15, 20, 22, 23, 27, 33, 34, 35, 36, 37, 38, 39, 40, 41, 42, 43, 44, 45, 46, 47, 48, 49, 50, 51, 52, 54, 58, 59, 60, 61, 63, 65, 66, 67, 68, 69, 70, 71, 72, 73, 74, 75, 77, 79, 81, 82, 83, 84, 85, 86, 87, 89, 90, 91, 92, 94, 96, 98, 99, 102, 103, 104, 105, 106, 107, 108, 110, 114, 117, 118, 119, 122, 124, 125, 127, 128, 130, 131, 132, 133, 134, 135, 136, 137, 138, 139, 143, 144, 145, 149, 150, 152, 153, 154, 155, 156, 157, 158, 159, 161, 163, 164, 165, 166, 167, 168, 169, 170, 171, 172, 173, 174, 175, 176, 177, 178, 179, 180, 181, 182, 183, 184, 186, 192, 193, 195, 197, 198, 200, 202, 204, 206, 208, 209, 211, 212, 213, 214, 215, 216, 217, 218, 219, 220, 221, 222, 224, 226, 230, 231, 232, 233, 234, 235, 236, 238, 239, 240, 242, 245, 247, 248, 251, 253, 255, 256, 257, 258, 260, 261, 263, 264, 265, 266, 267, 269, 270, 271, 272, 273, 274, 275, 276, 278, 279, 280, 282, 283, 284, 285, 286, 287, 288, 289, 290, 291, 292, 293, 294, 297, 298, 300, 301, 302, 304, 305, 306, 307, 308, 310, 311, 312, 313, 314, 315, 316, 317, 318, 320, 321, 323, 324, 325, 326, 327, 331, 334, 335, 338, 339, 342, 343, 347, 348, 349, 351, 353, 356, 357, 358, 359, 360, 361, 367, 368, 369, 371, 373, 374, 375, 376, 377, 378, 379, 380, 381, 383, 385, 387, 388, 389, 391, 392, 393, 394, 395, 396, 397, 398, 399, 401, 403, 404, 405, 406, 410, 411, 412, 413, 414, 415, 416, 417, 419, 420, 422, 425, 426, 427, 428, 431, 432, 434, 437, 438, 439, 440, 441, 442, 443, 444, or 447 with reference to amino acid positions of the sequence set forth in SEQ ID NO:3, the modification(s) is/are not the corresponding amino acid replacement(s) set forth in Table 8 herein, which are amino acid replacements that result in an inactive polypeptide. For example, if the modification is a modification at a position corresponding to position 2 with reference to SEQ ID NO:3, the modification is not replacement to a histidine (H), lysine (K), tryptophan (W) or tyrosine (Y).

For purposes herein, reference to positions and amino acids for modification herein, including amino acid replacement or replacements, are with reference to the PH20 polypeptide set forth in SEQ ID NO:3. It is within the level of one of skill in the art to make any of the modifications provided herein in another PH20 polypeptide by identifying the corresponding amino acid residue in another PH20 polypeptide, such as any set forth in SEQ ID NOS: 6, 7, 8, 9, 10, 11, 12, 13, 14, 15, 16, 17, 18, 19, 20, 21, 22, 23, 24-27, 28, 29, 30, 31, 32-66, 68-72, 387-392, 399 or 400 or a variant thereof that exhibits at least 70%, 75%, 80%, 85%, 86%, 87%, 88%, 89%, 90%, 91%, 92%, 93%, 94%, 95%, 96%, 97%, 98%, 99% or more sequence identity to any of SEQ ID NOS: 6, 7, 8, 9, 10, 11, 12, 13, 14, 15, 16, 17, 18, 19, 20, 21, 22, 23, 24-27, 28, 29, 30, 31, 32-66, 68-72, 387-392, 399 or 400. Corresponding positions in another PH20 polypeptide can be identified by alignment of the PH20 polypeptide with the reference to the PH20 polypeptide set forth in SEQ ID NO:3. For example, FIG. 2 (A-L) depicts alignment of exemplary PH20 polypeptides with SEQ ID NO:3, and identification of exemplary corresponding positions. Also, since SEQ ID NOS: 3, 7, 32-66, 69 and 72 are all forms of a mature human PH20 with a different C-terminal amino acid residue, the numbering of amino acid residues in any of SEQ ID NOS: 7, 32-66, 69 and 72 is the same as SEQ ID NO:3, and hence the corresponding residues of each are identical to that set forth in SEQ ID NO:3 (see e.g., FIG. 1). Further, SEQ ID NOS set forth in any of SEQ ID NOS: 2, 6, 70 or 71 are precursor forms thereof that differ by only the presence of a signal sequence.

For purposes of modification (e.g., amino acid replacement), the corresponding amino acid residue that is replaced can be any amino acid residue, and need not be identical to the residue set forth in SEQ ID NO:3. Typically, the corresponding amino acid residue identified by alignment with residues in SEQ ID NO:3 is an amino acid residue that is identical to SEQ ID NO:3, or is a conservative or semi-conservative amino acid residue thereto (see e.g., FIGS. 2A-2L). It is also understood that the exemplary replacements provided herein can be made at the corresponding residue in a PH20 polypeptide, so long as the replacement is different than exists in the unmodified form of the PH20 polypeptide. Based on this description and the description elsewhere herein, it is within the level of one of skill in the art to generate a modified PH20 polypeptide containing any one or more of the described mutation, and test each for a property or activity as described herein.

Modifications in a PH20 polypeptide also can be made to a PH20 polypeptide that also contains other modifications, including modifications of the primary sequence and modifications not in the primary sequence of the polypeptide. For example, modifications described herein can be in a PH20 polypeptide that is a fusion polypeptide or chimeric polypeptide. The modified PH20 polypeptides provided herein also include polypeptides that are conjugated to a polymer, such as a PEG reagent.

In the subsections below, exemplary modified PH20 uber-thermophile polypeptides exhibiting increased thermal stability, and encoding nucleic acid molecules, provided herein are described.

1. Exemplary Amino Acid Replacements

The uber-thermophile PH20 polypeptides provided herein can contain any amino acid replacement or amino acid replacements in an unmodified PH20 polypeptide as set forth in Table 3. For example, the uber-thermophile PH20 polypeptide can contain only a single amino acid replacement in an unmodified PH20 polypeptide as set forth in Table 3. In other examples, the uber-thermophile PH20 polypeptide can contain any two or more, such as three or more, for example at least 4, 5, 6, 7, 8, 9, 10, 11, 12, 13, 14, 15, 16, 17, 18, 19, 20 or more amino acid replacements in an unmodified PH20 polypeptide as set forth in Table 3. The unmodified PH20 polypeptide can be a full-length PH20 or a soluble C-terminal truncated fragment thereof set forth in any of SEQ ID NOS: 3-66, 68-72, 387-392, 399 or 400, or a polypeptide that exhibits at least 85%, 86%, 87%, 88%, 89%, 90%, 91%, 92%, 93%, 94%, 95%, 96%, 97%, 98%, 99% to any of SEQ ID NOS: 3-66, 68-72, 387-392, 399 or 400. In particular the modified PH20 polypeptide is a soluble C-terminal truncated PH20 polypeptide set forth in any of SEQ ID NOS: 3 or 32-66 or exhibits at least 85%, 86%, 87%, 88%, 89%, 90%, 91%, 92%, 93%, 94%, 95%, 96%, 97%, 98%, 99% to any of SEQ ID NOS: 3 or 32-66. Such modified PH20 polypeptides include those that retain at least 50%, 55%, 60%, 65%, 70%, 75%, 80%, 85%, 90%, 95% or greater of its hyaluronidase activity after incubation at 52° C. for 10 minutes compared to its hyaluronidase activity after incubation at 4° C. for 10 minutes (see e.g. Example 7 and Tables 10 and 11).

TABLE 3
Active Mutants

| Corresponding | | Corresponding | | Corresponding | |
|---|---|---|---|---|---|
| Position | Replacement | Position | Replacement | Position | Replacement |
| 10 | G N | 11 | G | 13 | H |
| 15 | A V | 26 | P R S V W Y | 27 | E H |
| 28 | L | 29 | E H L S W | 30 | A P R |
| 31 | C G L | 32 | Q S V W | 33 | G M R W |
| 34 | E H W | 36 | G | 37 | I K |
| 38 | Y | 39 | Q R T | 41 | D T W |
| 46 | H | 47 | G R | 48 | G Y |
| 49 | I | 50 | C D | 58 | K R |
| 60 | K | 67 | F | 69 | A Y |
| 72 | D | 73 | T | 83 | G Q V |
| 84 | D | 86 | D E N R | 87 | M P V |
| 90 | E T W | 92 | V | 93 | E S |
| 94 | N | 97 | E F | 98 | M |
| 99 | S | 102 | H N | 105 | I R W |
| 114 | G | 118 | M | 120 | S |
| 131 | C L | 132 | A C | 135 | Q |
| 138 | W | 139 | R V | 141 | M Q W Y |
| 142 | Q | 143 | K | 144 | G |
| 146 | V | 147 | G I M | 148 | C H K |
| 150 | L | 151 | Q | 152 | A I M T |
| 154 | R | 155 | A D F H L R S V | 156 | A C Q |
| 158 | H | 159 | A H N Q S | 160 | Y |
| 161 | A D | 162 | L | 163 | K R S |
| 165 | F | 170 | R | 174 | W |
| 195 | H L N | 196 | T | 197 | F |
| 198 | L | 202 | M | 204 | P |
| 205 | A E K L P S T | 206 | I | 208 | L Q R |
| 213 | E N | 215 | A D E H T V W | 219 | A R S T |
| 220 | V | 222 | N | 234 | M |
| 235 | T | 237 | Q | 240 | Q |
| 247 | I | 251 | L M | 255 | R |
| 259 | K P | 260 | G M | 261 | A F |
| 263 | T | 265 | I | 271 | V |
| 276 | E | 277 | A C E H | 278 | G H K N |
| 282 | G Q | 284 | A Q S | 285 | M Y |
| 290 | M | 292 | V | 305 | D N |
| 306 | D | 309 | E H L | 310 | Q R |
| 311 | G K | 315 | T | 317 | N |
| 318 | K M N Q | 320 | R | 321 | A H R |
| 328 | L R | 342 | A | 343 | V |
| 349 | A E | 359 | E | 368 | H K |
| 369 | H | 371 | E F M T | 373 | S |
| 374 | A V | 375 | T | 376 | Y |
| 377 | T | 379 | H S T | 380 | I L P T V |
| 388 | H | 389 | K | 393 | L |
| 399 | R W | 401 | G | 403 | F |
| 406 | N | 407 | F H M P Q | 410 | S |
| 412 | G P S | 413 | Q T | 415 | W |
| 417 | L | 419 | L | 421 | I M |
| 428 | P | 431 | A G | 433 | L T |
| 434 | I M | 435 | H | 438 | A |
| 439 | C T | 440 | M | 441 | T V |
| 442 | P | 443 | M | 445 | Y |
| 446 | C D E G | 447 | D E G | | |

In examples herein, a modified PH20 polypeptide contains an amino acid replacement that is one or more of replacement with: H at a position corresponding to position 27; H at a position corresponding to position 29; W at a position corresponding to position 34; K at a position corresponding to position 37; G at a position corresponding to position 48; K at a position corresponding to position 58; R at a position corresponding to position 58; H at a position corresponding to position 102; Y at a position corresponding to position 141; K at a position corresponding to position 143; G at a position corresponding to position 144; I at a position corresponding to position 147; D at a position corresponding to position 155; N at a position corresponding to position 159; F at a position corresponding to position 165; W at a position corresponding to position 174; P at a position corresponding to position 204; E at a position corresponding to position 213; T at a position corresponding to position 215; A at a position corresponding to position 205; I at a position corresponding to position 206; T at a position corresponding to position 235; A at a position corresponding to position 261; F at a position corresponding to position 261; A at a position corresponding to position 277; C at a position corresponding to position 277; A at a position corresponding to position 284; S at a position corresponding to position 284; D at a position corresponding to position 306; G at a position corresponding to position 311; T at a position corresponding to position 315; H at a position corresponding to position 369; L at a position corresponding to position 380; or S at a position corresponding to position 412, with reference to positions in any of SEQ ID NOS: 3, 7 or 32-66.

For example, in examples herein, exemplary amino acid replacements in the modified PH20 polypeptides provided herein include, but are not limited to, replacement with: glycine (G) at a position corresponding to position 11; A at a position corresponding to position 15; V at a position corresponding to position 15; R at a position corresponding to position 26; S at a position corresponding to position 26; E at a position corresponding to position 27; H at a position corresponding to position 27; H at a position corresponding to position 29; S at a position corresponding to position 29; A at a position corresponding to position 30; P at a position corresponding to position 30; G at a position corresponding to position 31; L at a position corresponding to position 31; Q at a position corresponding to position 32; W at a position corresponding to position 32; G at a position corresponding to position 33; M at a position corresponding to position 33; R at a position corresponding to position 33; W at a position corresponding to position 33; E at a position corresponding to position 34; H at a position corresponding to position 34; W at a position corresponding to position 34; K at a position corresponding to position 37; Y at a position corresponding to position 38; Q at a position corresponding to position 39; R at a position corresponding to position 39; T at a position corresponding to position 39; D at a position corresponding to position 41; T at a position corresponding to position 41; W at a position corresponding to position 41; G at a position corresponding to position 48; C at a position corresponding to position 50; D at a position corresponding to position 50; K at a position corresponding to position 58; R at a position corresponding to position 58; K at a position corresponding to position 60; F at a position corresponding to position 67; A at a position corresponding to position 69; Y at a position corresponding to position 69; Q at a position corresponding to position 83; D at a position corresponding to position 84; D at a position corresponding to position 86; E at a position corresponding to position 86; R at a position corresponding to position 86; P at a position corresponding to position 87; W at a position corresponding to position 90; V at a position corresponding to position 92; E at a position corresponding to position 93; S at a position corresponding to position 93; N at a position corresponding to position 94; F at a position corresponding to position 97; M at a position corresponding to position 98; S at a position corresponding to position 99; H at a position corresponding to position 102; G at a position corresponding to position 114; M at a position corresponding to position 118; S at a position corresponding to position 120; C at a position corresponding to position 131; L at a position corresponding to position 131; A at a position corresponding to position 132; W at a position corresponding to position 138; R at a position corresponding to position 139; V at a position corresponding to position 139; M at a position corresponding to position 141; Y at a position corresponding to position 141; K at a position corresponding to position 143; G at a position corresponding to position 144; V at a position corresponding to position 146; I at a position corresponding to position 147; M at a position corresponding to position 147; C at a position corresponding to position 148; H at a position corresponding to position 148; K at a position corresponding to position 148; L at a position corresponding to position 150; Q at a position corresponding to position 151; I at a position corresponding to position 152; M at a position corresponding to position 152; T at a position corresponding to position 152; R at a position corresponding to position 154; A at a position corresponding to position 155; D at a position corresponding to position 155; F at a position corresponding to position 155; H at a position corresponding to position 155; L at a position corresponding to position 155; R at a position corresponding to position 155; S at a position corresponding to position 155; H at a position corresponding to position 158; A at a position corresponding to position 159; H at a position corresponding to position 159; N at a position corresponding to position 159; Q at a position corresponding to position 159; S at a position corresponding to position 159; Y at a position corresponding to position 160; A at a position corresponding to position 161; L at a position corresponding to position 162; K at a position corresponding to position 163; R at a position corresponding to position 163; S at a position corresponding to position 163; F at a position corresponding to position 165; W at a position corresponding to position 174; H at a position corresponding to position 195; L at a position corresponding to position 195; T at a position corresponding to position 196; F at a position corresponding to position 197; L at a position corresponding to position 198; P at a position corresponding to position 204; A at a position corresponding to position 205; E at a position corresponding to position 205; K at a position corresponding to position 205; L at a position corresponding to position 205; T at a position corresponding to position 205; I at a position corresponding to position 206; Q at a position corresponding to position 208; R at a position corresponding to position 208; E at a position corresponding to position 213; N at a position corresponding to position 213; E at a position corresponding to position 215; H at a position corresponding to position 215; T at a position corresponding to position 215; N at a position corresponding to position 222; T at a position corresponding to position 235; Q at a position corresponding to position 237; Q at a position corresponding to position 240; I at a position corresponding to position 247; L at a position corresponding to position 251; M at a position corresponding to position 251; K at a position corresponding to position 259; P at a position corresponding to position 259; M at a position corresponding to position 260; A at a position corresponding to position 261; F at a position corresponding to position 261; T at a position corresponding to position 263; V at a position corresponding to position 271; E at a position corresponding to position 276; A at a position corresponding to position 277; C at a position corresponding to position 277; N at a position corresponding to position 278; Q at a position corresponding to position 282; A at a position corresponding to position 284; Q at a position corresponding to position 284; S at a position corresponding to position 284; M at a position corresponding to position 285; V at a position corresponding to position 292; N at a position corresponding to position 305; D at a position corresponding to position 306; R at a position corresponding to position 310; G at a position corresponding to position 311; T at a position corresponding to position 315; N at a position corresponding to position 317; A at a position corresponding to position 321; R at a position corresponding to position 321; L at a position corresponding to position 328; R at a position corresponding to position 328; A at a position corresponding to position 342; H at a position corresponding to position 368; K at a position corresponding to position 368; H at a position corresponding to position 369; F at a position corresponding to position 371; S at a position corresponding to position 373; T at a position corresponding to position 377; H at a position corresponding to position 379; S at a position corresponding to position 379; T at a position corresponding to position 379; I at a position corresponding to position 380; L at a position corresponding to position 380; P at a position corresponding to position 380; T at a position corresponding to position 380; H at a position corresponding to position 388; N at a position corresponding to position 406; F at a position corresponding to position 407; Q at a position corresponding to position 407; S at a position corresponding to position 410; G at a position corresponding to position 412; P at a position corresponding to position 412; S at a position corresponding to position 412; Q at a position corresponding to position 413; M at a position corresponding to position 421; P at a position corresponding to position 428; A at a position corresponding to position 431; L at a position corresponding to position 433; T at a position corresponding to position 433; A at a position corresponding to position 438; C at a position corresponding to position 439; T at a position corresponding to position 441; M at a position corresponding to position 443; Y at a position corresponding to position 445; C at a position corresponding to position 446; D at a position corresponding to position 446; E at a position corresponding to position 446; G at a position corresponding to position 446; E at a position corresponding to position 447; and G at a position corresponding to position 447, with reference to positions in any of SEQ ID NOS: 3, 7 or 32-66.

For example, in examples herein, a modified PH20 polypeptide contains an amino acid replacement that is one or more of replacement with: H at a position corresponding to position 29; K at a position corresponding to position 37; G at a position corresponding to position 48; R at a position corresponding to position 58; K at a position corresponding to position 143; I at a position corresponding to position 147; N at a position corresponding to position 159; P at a position corresponding to position 204; I at a position corresponding to position 206; T at a position corresponding to position 235; A at a position corresponding to position 261; F at a position corresponding to position 261; A at a position corresponding to position 284; D at a position corresponding to position 306; G at a position corresponding to position 311; T at a position corresponding to position 315; H at a position corresponding to position 369; or S at a position corresponding to position 412, with reference to positions in any of SEQ ID NOS: 3, 7 or 32-66.

Exemplary amino acid replacements in the modified PH20 polypeptides provided herein include, but are not limited to, replacement with: alanine (A) at a position corresponding to position 15; V at a position corresponding to position 15; R at a position corresponding to position 26; E at a position corresponding to position 27; S at a position corresponding to position 29; G at a position corresponding to position 31; L at a position corresponding to position 31; Q at a position corresponding to position 32; G at a position corresponding to position 33; M at a position corresponding to position 33; R at a position corresponding to position 33; W at a position corresponding to position 33; E at a position corresponding to position 34; H at a position corresponding to position 34; Y at a position corresponding to position 38; R at a position corresponding to position 39; W at a position corresponding to position 41; G at a position corresponding to position 48; C at a position corresponding to position 50; R at a position corresponding to position 58; A at a position corresponding to position 69; D at a position corresponding to position 86; E at a position corresponding to position 86; R at a position corresponding to position 86; W at a position corresponding to position 90; E at a position corresponding to position 93; S at a position corresponding to position 93; F at a position corresponding to position 97; S at a position corresponding to position 99; S at a position corresponding to position 120; L at a position corresponding to position 131; A at a position corresponding to position 132; W at a position corresponding to position 138; R at a position corresponding to position 139; V at a position corresponding to position 139; M at a position corresponding to position 141; Y at a position corresponding to position 141; K at a position corresponding to position 143; V at a position corresponding to position 146; I at a position corresponding to position 147; M at a position corresponding to position 147; C at a position corresponding to position 148; H at a position corresponding to position 148; K at a position corresponding to position 148; L at a position corresponding to position 150; Q at a position corresponding to position 151; I at a position corresponding to position 152; M at a position corresponding to position 152; T at a position corresponding to position 152; R at a position corresponding to position 154; A at a position corresponding to position 155; F at a position corresponding to position 155; L at a position corresponding to position 155; R at a position corresponding to position 155; H at a position corresponding to position 158; H at a position corresponding to position 159; N at a position corresponding to position 159; Q at a position corresponding to position 159; S at a position corresponding to position 159; Y at a position corresponding to position 160; R at a position corresponding to position 163; F at a position corresponding to position 165; W at a position corresponding to position 174; L at a position corresponding to position 198; P at a position corresponding to position 204; A at a position corresponding to position 205; L at a position corresponding to position 205; T at a position corresponding to position 205; I at a position corresponding to position 206; Q at a position corresponding to position 208; R at a position corresponding to position 208; N at a position corresponding to position 213; E at a position corresponding to position 215; T at a position corresponding to position 215; T at a position corresponding to position 235; Q at a position corresponding to position 237; Q at a position corresponding to position 240; L at a position corresponding to position 251; K at a position corresponding to position 259; M at a position corresponding to position 260; A at a position corresponding to position 261; F at a position corresponding to position 261; T at a position corresponding to position 263; V at a position corresponding to position 271; E at a position corresponding to position 276; A at a position corresponding to position 277; C at a position corresponding to position 277; A at a position corresponding to position 284; Q at a position corresponding to position 284; S at a position corresponding to position 284; V at a position corresponding to position 292; N at a position corresponding to position 305; D at a position corresponding to position 306; R at a position corresponding to position 310; T at a position corresponding to position 315; R at a position corresponding to position 328; A at a position corresponding to position 342; K at a position corresponding to position 368; H at a position corresponding to position 369; S at a position corresponding to position 373; H at a position corresponding to position 379; S at a position corresponding to position 379; T at a position corresponding to position 379; I at a position corresponding to position 380; L at a position corresponding to position 380; P at a position corresponding to position 380; T at a position corresponding to position 380; H at a position corresponding to position 388; G at a position corresponding to position 412; P at a position corresponding to position 412; S at a position corresponding to position 412; Q at a position corresponding to position 413; T at a position corresponding to position 433; A at a position corresponding to position 438; T at a position corresponding to position 441; M at a position corresponding to position 443; Y at a position corresponding to position 445; C at a position corresponding to position 446; E at a position corresponding to position 447; and G at a position corresponding to position 447, with reference to positions in any of SEQ ID NOS: 3, 7 or 32-66.

For example, in examples herein, a modified PH20 polypeptide contains an amino acid replacement that is one or more of replacement with: K at a position corresponding to position 143; I at a position corresponding to position 147; P at a position corresponding to position 204; T at a position corresponding to position 235; A at a position corresponding to position 261; A at a position corresponding to position 284; D at a position corresponding to position 306; T at a position corresponding to position 315; or H at a position corresponding to position 369, with reference to positions in any of SEQ ID NOS: 3, 7 or 32-66.

In examples herein, exemplary amino acid replacements in the modified PH20 polypeptides provided herein include, but are not limited to, replacement with: alanine (A) at a position corresponding to position 15; V at a position corresponding to position 15; R at a position corresponding to position 26; E at a position corresponding to position 27; S at a position corresponding to position 29; G at a position corresponding to position 31; G at a position corresponding to position 33; M at a position corresponding to position 33; R at a position corresponding to position 33; W at a position corresponding to position 33; E at a position corresponding to position 34; H at a position corresponding to position 34; Y at a position corresponding to position 38; R at a position corresponding to position 39; G at a position corresponding to position 48; R at a position corresponding to position 86; W at a position corresponding to position 90; E at a position corresponding to position 93; S at a position corresponding to position 93; F at a position corresponding to position 97; S at a position corresponding to position 120; L at a position corresponding to position 131; A at a position corresponding to position 132; R at a position corresponding to position 139; M at a position corresponding to position 141; Y at a position corresponding to position 141; K at a position corresponding to position 143; I at a position corresponding to position 147; M at a position corresponding to position 147; C at a position corresponding to position 148; H at a position corresponding to position 148; K at a position corresponding to position 148; M at a position corresponding to position 152; T at a position corresponding to position 152; R at a position corresponding to position 154; A at a position corresponding to position 155; F at a position corresponding to position 155; L at a position corresponding to position 155; N at a position corresponding to position 159; S at a position corresponding to position 159; Y at a position corresponding to position 160; R at a position corresponding to position 163; F at a position corresponding to position 165; W at a position corresponding to position 174; L at a position corresponding to position 198; P at a position corresponding to position 204; A at a position corresponding to position 205; L at a position corresponding to position 205; T at a position corresponding to position 205; I at a position corresponding to position 206; R at a position corresponding to position 208; N at a position corresponding to position 213; E at a position corresponding to position 215; T at a position corresponding to position 215; Q at a position corresponding to position 240; L at a position corresponding to position 251; K at a position corresponding to position 259; M at a position corresponding to position 260; A at a position corresponding to position 261; F at a position corresponding to position 261; T at a position corresponding to position 263; Vat a position corresponding to position 271; A at a position corresponding to position 277; C at a position corresponding to position 277; A at a position corresponding to position 284; Q at a position corresponding to position 284; S at a position corresponding to position 284; V at a position corresponding to position 292; T at a position corresponding to position 315; A at a position corresponding to position 342; H at a position corresponding to position 369; H at a position corresponding to position 379; S at a position corresponding to position 379; T at a position corresponding to position 379; L at a position corresponding to position 380; P at a position corresponding to position 380; T at a position corresponding to position 380; H at a position corresponding to position 388; G at a position corresponding to position 412; P at a position corresponding to position 412; S at a position corresponding to position 412; T at a position corresponding to position 433; A at a position corresponding to position 438; T at a position corresponding to position 441; M at a position corresponding to position 443; Y at a position corresponding to position 445; C at a position corresponding to position 446; E at a position corresponding to position 447; and G at a position corresponding to position 447, with reference to positions in any of SEQ ID NOS: 3, 7 or 32-66.

In examples herein, exemplary amino acid replacements in the modified PH20 polypeptides provided herein include, but are not limited to, replacement with: glutamic acid (E) at a position corresponding to position 27; A at a position corresponding to position 132; K at a position corresponding to position 143; M at a position corresponding to position 147; C at a position corresponding to position 148; H at a position corresponding to position 148; Y at a position corresponding to position 160; P at a position corresponding to position 204; A at a position corresponding to position 205; I at a position corresponding to position 206; T at a position corresponding to position 215; M at a position corresponding to position 260; A at a position corresponding to position 261; F at a position corresponding to position 261; T at a position corresponding to position 263; A at a position corresponding to position 284; T at a position corresponding to position 315; and S at a position corresponding to position 379, with reference to positions in any of SEQ ID NOS: 3, 7 or 32-66.

In examples herein, a modified PH20 polypeptide contains an amino acid replacement that is one or more of replacement with: P at a position corresponding to position 30; R at a position corresponding to position 58; K at a position corresponding to position 60; K at a position corresponding to position 143; I at a position corresponding to position 147; P at a position corresponding to position 204; T at a position corresponding to position 215; T at a position corresponding to position 235; A at a position corresponding to position 261; G at a position corresponding to position 311; T at a position corresponding to position 315; and H at a position corresponding to position 369, with reference to positions in any of SEQ ID NOS: 3, 7 or 32-66.

In particular, in examples herein, a modified PH20 polypeptide contains an amino acid replacement that is one or more of replacement with: P at a position corresponding to position 204; A at a position corresponding to position 284; or T at a position corresponding to position 315, with reference to positions in any of SEQ ID NOS: 3, 7 or 32-66.

Provided herein are modified PH20 polypeptides set forth in any of SEQ ID NOS: 73-386, or a polypeptide that exhibits at least 85%, 86%, 87%, 88%, 89%, 90%, 91%, 92%, 93%, 94%, 95%, 96%, 97%, 98%, 99% or more sequence identity to any of SEQ ID NOS: 73-386.

2. Nucleic Acid Molecules

Also provided herein are nucleic acid molecules that encode any of the modified PH20 polypeptides provided herein. For example, provided herein are nucleic acid molecules that encode any of the modified PH20 polypeptides set forth in any of SEQ ID NOS: 73-386, or that encodes a polypeptide that exhibits at least 85%, 86%, 87%, 88%, 89%, 90%, 91%, 92%, 93%, 94%, 95%, 96%, 97%, 98%, 99% or more sequence identity to any of SEQ ID NOS: 73-386.

In particular examples, the nucleic acid sequence can be codon optimized, for example, to increase expression levels of the encoded sequence. The particular codon usage is dependent on the host organism in which the modified polypeptide is expressed. One of skill in the art is familiar with optimal codons for expression in mammalian or human cells, bacteria or yeast, including for example E. coli or Saccharomyces cerevisiae. For example, codon usage information is available from the Codon Usage Database available at kazusa.or.jp.codon (see Richmond (2000) Genome Biology, 1:reports241 for a description of the database. See also, Forsburg (1994) Yeast, 10:1045-1047; Brown et al. (1991) Nucleic Acids Research, 19:4298; Sharp et al. (1988) Nucleic Acids Res., 12:8207-8211; Sharp et al. (1991) Yeast, 657-78). In some examples, the encoding nucleic acid molecules also can be modified to contain a heterologous signal sequence to alter (e.g., increased) expression and secretion of the polypeptide. Exemplary of a heterologous signal sequence is a nucleic acid encoding the IgG kappa signal sequence (set forth in SEQ ID NO:398).

The modified polypeptides and encoding nucleic acid molecules provided herein can be produced by standard recombinant DNA techniques known to one of skill in the art. Any method known in the art to effect mutation of any one or more amino acids in a target protein can be employed. Methods include standard site-directed or random mutagenesis of encoding nucleic acid molecules, or solid phase polypeptide synthesis methods. For example, nucleic acid molecules encoding a PH20 polypeptide can be subjected to mutagenesis, such as random mutagenesis of the encoding nucleic acid, error-prone PCR, site-directed mutagenesis, overlap PCR, gene shuffling, or other recombinant methods. The nucleic acid encoding the polypeptides can then be introduced into a host cell to be expressed heterologously. Hence, also provided herein are nucleic acid molecules encoding any of the modified polypeptides provided herein. In some examples, the modified PH20 polypeptides are produced synthetically, such as using solid phase or solutions phase peptide synthesis.

3. Additional Modifications and Conjugates

The modified PH20 polypeptides include those that contain chemical or posttranslational modifications. In some examples, modified PH20 polypeptides provided herein do not contain chemical or posttranslational modifications. Chemical and post-translational modifications include, but are not limited to, PEGylation, sialation, albumination, glycosylation, farnysylation, carboxylation, hydroxylation, phosphorylation, and other polypeptide modifications known in the art.

Also, in addition to any one or more amino acid modifications, such as amino acid replacements, provided herein, modified PH20 polypeptides provided herein can be conjugated or fused to any moiety using any method known in the art, including chemical and recombinant methods, provided the resulting polypeptide retains hyaluronidase activity. For example, in addition to any one or more amino acid modifications, such as amino acid replacements, provided herein, modified PH20 polypeptides provided herein also can contain other modifications that are or are not in the primary sequence of the polypeptide, including, but not limited to, modification with a carbohydrate moiety, a polyethylene glycol (PEG) moiety, a sialic acid moiety, an Fc domain from immunoglobulin G, or any other domain or moiety. For example, such additional modifications can be made to increase the stability or serum half-life of the protein.

In some instances, the domain or other moiety is a targeted agent, including any agent that targets the conjugate to one or more cell types by selectively binding to a cell surface receptor or other cell surface moiety. For example, the domain or other moiety is a targeted agent that targets the conjugate to tumor cells. In such examples, a modified PH20 polypeptide, such as any provided herein, is linked directly or indirectly to a targeted agent. Such targeting agents include, but are not limited to, growth factors, cytokines, chemokines, antibodies, and hormones, or allelic variants, muteins, or fragments thereof so long as the targeting agent is internalized by a cell surface receptor. Exemplary, non-limiting, additional modifications are described below.

a. Decreased immunogenicity

The modified PH20 polypeptides provided herein can be made to have decreased immunogenicity. Decreased immunogenicity can be effected by sequence changes that elimiminate antigenic epitopes from the polypeptide or by altering post-translational modifications. One of skill in the art is familiar with methods of identifiying antigenic epitopes in a polypeptide (see e.g., Liang et al. (2009) BMC Bioinformatics, 10:302; Yang et al. (2009) Rev. Med. Virol., 19:77-96). In some examples, one or more amino acids can be modified in order to remove or alter an antigenic epitope.

In another example, altering the glycosylation of a protein also can effect immunogenicity. For example, altering the glycosylation of the peptide is contemplated, so long as the polypeptides minimally contain at least N-acetylglucosamine at amino acid residues corresponding to amino acid residues set forth as N200, N333 and N358 of SEQ ID NO:3 or 7.

For example, the PH20 polypeptides can be modified such that they lack fucose, particularly bifucosylation. In particular, the PH20 polypeptides provided herein are not bifucosylated. This can be achieved by expressing and producing the PH20 polypeptide in host cells that do not effect bifucosylation. Fucose is a deoxyhexose that is present in a wide variety of organisms, including mammals, insects and plants. Fucosylated glycans are synthesized by fucosyl-transferases; see, e.g., Ma et al., Glycobiology, 16(12):158R-184R, (2006); Nakayama et al., J. Biol. Chem., 276:16100-16106 (2001); and Sturla et al., Glycobiology, 15(10):924-935 (2005). In humans, fucose frequently exists as a terminal modification to glycan structures, and the presence of fucose α1,6-linked to N-acetylglucosamine has been shown to be important in glycoprotein processing and recognition. In insects, N-glycan core structures exhibit bifucosylation with α1,6- and α1,3-linkages. Insect cell core fucosylation with α1,3-linkages generates a carbohydrate epitope that is immunogenic in humans (see, e.g., US Publication No. 20070067855). For example, PH20 polypeptides provided herein can be generated in host cells that are incapable of bifucosylating the polypeptide. Thus, while insect cells or other cells that bifucosylate can be used for expression of the polypeptides, typically mammalian cells, such as CHO cells, are used.

In some examples, defucosylated, or fucose-deficient PH20 polypeptides can be generated in insect cells with modified glycosylation pathways, through the use of baculovirus expression vectors containing eukaryotic oligosaccharide processing genes, thereby creating “mammalianized” insect cell expression systems (see, e.g., U.S. Pat. No. 6,461,863). Alternatively, antigenicity can be eliminated by expression of PH20 polypeptides in insect cells lacking α1,3-fucosylatransferase (FT3) (see, e.g., US Publication No. 20070067855). In other examples, defucosylated or fucose-deficient PH20 polypeptides can be generated, for example, in cell lines that produce defucosylated proteins, including Lec13 CHO cells deficient in protein fucosylation (Ripka et al. Arch. Biochem. Biophys. 249:533-545 (1986); U.S. Pat. Pub. No. 2003/0157108; and WO 2004/056312), and knockout cell lines, such as alpha-1,6-fucosyltransferase gene, FUT8, knockout CHO cells (Yamane-Ohnuki et al. Biotech. Bioeng. 87: 614 (2004)).

B. Conjugation to Polymers

In some examples, the modified PH20 polypeptides provided herein are conjugated to polymers. Exemplary polymers that can be conjugated to the PH20 polypeptides, include natural and synthetic homopolymers, such as polyols (i.e., poly-OH), polyamines (i.e., poly-NH₂) and polycarboxylic acids (i.e., poly-COOH), and further heteropolymers, i.e., polymers containing one or more different coupling groups, e.g., hydroxyl groups and amine groups. Examples of suitable polymeric molecules include polymeric molecules selected from among polyalkylene oxides (PAO), such as polyalkylene glycols (PAG), including polyethylene glycols (PEG), methoxypolyethylene glycols (mPEG) and polypropylene glycols, PEG-glycidyl ethers (Epox-PEG), PEG-oxycarbonylimidazole (CDI-PEG), branched polyethylene glycols (PEGs), polyvinyl alcohol (PVA), polycarboxylates, polyvinylpyrrolidone, poly-D,L-amino acids, polyethylene-co-maleic acid anhydride, polystyrene-co-maleic acid anhydride, dextrans including carboxymethyl-dextrans, heparin, homologous albumin, celluloses, including methylcellulose, carboxymethylcellulose, ethylcellulose, hydroxyethylcellulose, carboxyethylcellulose and hydroxypropylcellulose, hydrolysates of chitosan, starches such as hydroxyethyl-starches and hydroxypropyl-starches, glycogen, agaroses and derivatives thereof, guar gum, pullulan, inulin, xanthan gum, carrageenan, pectin, alginic acid hydrolysates and bio-polymers.

Typically, the polymers are polyalkylene oxides (PAO), such as polyethylene oxides, such as PEG, typically mPEG, which have few reactive groups capable of cross-linking. Typically, the polymers are non-toxic polymeric molecules such as (methoxy)polyethylene glycol (mPEG) which can be covalently conjugated to the PH20 polypeptides (e.g., to attachment groups on the protein surface) using a relatively simple chemistry.

Suitable polymeric molecules for attachment to the PH20 polypeptides include, but are not limited to, polyethylene glycol (PEG) and PEG derivatives such as methoxy-polyethylene glycols (mPEG), PEG-glycidyl ethers (Epox-PEG), PEG-oxycarbonylimidazole (CDI-PEG), branched PEGs, and polyethylene oxide (PEO) (see e.g., Roberts et al., Advanced Drug Delivery Review 2002, 54:459-476; Harris and Zalipsky (eds.) “Poly(ethylene glycol), Chemistry and Biological Applications” ACS Symposium Series 680, 1997; Mehvar et al., J. Pharm. Pharmaceut. Sci., 3(1):125-136, 2000; Harris and Chess (2003) Nat Rev Drug Discov. 2(3):214-21; and Tsubery, J Biol. Chem 279(37):38118-24, 2004). The polymeric molecule can be of a molecular weight typically ranging from about 3 kDa to about 60 kDa. In some embodiments the polymeric molecule that is conjugated to a PH20 polypeptide provided herein has a molecular weight of 5, 10, 15, 20, 25, 30, 35, 40, 45, 50, 55, 60 or more than 60 kDa.

Various methods of modifying polypeptides by covalently attaching (conjugating) a PEG or PEG derivative (i.e., “PEGylation”) are known in the art (see e.g., U.S. 2006/0104968; U.S. Pat. No. 5,672,662; U.S. Pat. No. 6,737,505; and U.S. 2004/0235734). Techniques for PEGylation include, but are not limited to, specialized linkers and coupling chemistries (see e.g., Roberts, Adv. Drug Deliv. Rev. 54:459-476, 2002), attachment of multiple PEG moieties to a single conjugation site (such as via use of branched PEGs; see e.g., Guiotto et al., Bioorg. Med. Chem. Lett. 12:177-180, 2002), site-specific PEGylation and/or mono-PEGylation (see e.g., Chapman et al., Nature Biotech. 17:780-783, 1999), and site-directed enzymatic PEGylation (see e.g., Sato, Adv. Drug Deliv. Rev., 54:487-504, 2002) (see, also, for example, Lu and Felix (1994) Int. J. Peptide Protein Res. 43:127-138; Lu and Felix (1993) Peptide Res. 6:140-6, 1993; Felix et al. (1995) Int. J. Peptide Res. 46:253-64; Benhar et al. (1994) J. Biol. Chem. 269:13398-404; Brumeanu et al. (1995) J Immunol. 154:3088-95; see also, Caliceti et al. (2003) Adv. Drug Deliv. Rev. 55(10):1261-77 and Molineux (2003) Pharmacotherapy 23 (8 Pt 2):3S-8S). Methods and techniques described in the art can produce proteins having 1, 2, 3, 4, 5, 6, 7, 8, 9, 10 or more than 10 PEG or PEG derivatives attached to a single protein molecule (see e.g., U.S. 2006/0104968).

Numerous reagents for PEGylation have been described in the art. Such reagents include, but are not limited to, N-hydroxysuccinimidyl (NHS) activated PEG, succinimidyl mPEG, mPEG2-N-hydroxysuccinimide, mPEG succinimidyl alpha-methylbutanoate, mPEG succinimidyl propionate, mPEG succinimidyl butanoate, mPEG carboxymethyl 3-hydroxybutanoic acid succinimidyl ester, homobifunctional PEG-succinimidyl propionate, homobifunctional PEG propionaldehyde, homobifunctional PEG butyraldehyde, PEG maleimide, PEG hydrazide, p-nitrophenyl-carbonate PEG, mPEG-benzotriazole carbonate, propionaldehyde PEG, mPEG butryaldehyde, branched mPEG2 butyraldehyde, mPEG acetyl, mPEG piperidone, mPEG methylketone, mPEG “linkerless” maleimide, mPEG vinyl sulfone, mPEG thiol, mPEG orthopyridylthioester, mPEG orthopyridyl disulfide, Fmoc-PEG-NHS, Boc-PEG-NHS, vinylsulfone PEG-NHS, acrylate PEG-NHS, fluorescein PEG-NHS, and biotin PEG-NHS (see e.g., Monfardini et al., Bioconjugate Chem. 6:62-69, 1995; Veronese et al., J. Bioactive Compatible Polymers 12:197-207, 1997; U.S. Pat. No. 5,672,662; U.S. Pat. No. 5,932,462; U.S. Pat. No. 6,495,659; U.S. Pat. No. 6,737,505; U.S. Pat. No. 4,002,531; U.S. Pat. No. 4,179,337; U.S. Pat. No. 5,122,614; U.S. Pat. No. 5,324,844; U.S. Pat. No. 5,446,090; U.S. Pat. No. 5,612,460; U.S. Pat. No. 5,643,575; U.S. Pat. No. 5,766,581; U.S. Pat. No. 5,795,569; U.S. Pat. No. 5,808,096; U.S. Pat. No. 5,900,461; U.S. Pat. No. 5,919,455; U.S. Pat. No. 5,985,263; U.S. Pat. No. 5,990,237; U.S. Pat. No. 6,113,906; U.S. Pat. No. 6,214,966; U.S. Pat. No. 6,258,351; U.S. Pat. No. 6,340,742; U.S. Pat. No. 6,413,507; U.S. Pat. No. 6,420,339; U.S. Pat. No. 6,437,025; U.S. Pat. No. 6,448,369; U.S. Pat. No. 6,461,802; U.S. Pat. No. 6,828,401; U.S. Pat. No. 6,858,736; U.S. 2001/0021763; U.S. 2001/0044526; U.S. 2001/0046481; U.S. 2002/0052430; U.S. 2002/0072573; U.S. 2002/0156047; U.S. 2003/0114647; U.S. 2003/0143596; U.S. 2003/0158333; U.S. 2003/0220447; U.S. 2004/0013637; U.S. 2004/0235734; U.S. 2005/0114037; U.S. 2005/0171328; U.S. 2005/0209416; EP 1064951; EP 0822199; WO 01076640; WO 0002017; WO 0249673; WO 9428024; WO 0187925; and WO 2005000360).

### D. METHODS FOR IDENTIFYING THERMALLY STABLE HYALURONAN-DEGRADING ENZYMES

Provided herein are methods for identifying a modified or variant hyaluronan-degrading enzyme, such as a modified hyaluronidase or modified PH20 polypeptide, that exhibits thermal resistance compared to an unmodified hyaluronan-degrading enzyme, and is thermally stable. In the method, a modified hyaluronan-degrading enzyme or enzymes is/are tested or screened for hyaluronidase activity under a thermal stress condition (known to be destabilizing to a reference or unmodified hyalruonan-degrading enzyme) and are tested or screened for activity under a thermal neutral condition (known to be tolerated by a reference or unmodified hyaluronan-degrading enzyme).

In the method, one or more modified hyaluronan-degrading enzymes are provided. In some examples, a library of modified molecules is prepared. Methods of mutagenesis and generation of libraries or collections of variant molecules is described herein and is known to one of skill in the art using standard recombinant DNA techniques. In one example, the enzymes that are tested can be pooled and screened, whereby the method permits selection of only those enzymes that exhibit thermal resistance. In another example, the tested enzymes can be physically separated and screened individually, such as by formatting in arrays, such as addressable arrays.

Modified hyaluronan-degrading enzymes are identified that retain or exhibit at least 50% of the activity after incubation under the thermal stress condition compared to under the thermal neutral condition, such as generally at least 55%, 60%, 65%, 70%, 75%, 80%, 85%, 90%, 95% or more of the activity.

The method can be repeated a plurality of times. For example, the steps of the method can be repeated 1, 2, 3, 4, or 5 times. The method also can be performed iteratively, where an identified modified polypeptide is used as a reference polypeptide to generate a new collection of modified enzymes for screening. In one example, after the method is performed, any identified modified hyaluronan-degrading enzyme can be modified or further modified to increase or optimize the activity. By practice of the method, a thermally stable hyaluronan-degrading enzyme is identified.

A description of the steps of the method and components of the method are provided in the subsections that follow.

1. Hyaluronan-Degrading Enzymes and Libraries of Modified Hyaluronan-Degrading Enzymes

In the methods, one or more modified hyaluronan-degrading enzymes, such as a hyaluronidase or a PH20 polypeptide, are tested or screened. The modified hyaluronan-degrading enzyme can be modified compared to an unmodified hyaluronan-degrading enzyme, such as any hyaluronan-degrading enzyme known in the art. Hyaluronan-degrading enzymes are a family of enzymes that degrade hyaluronic acid, which is an essential component of the extracellular matrix and a major constituent of the interstitial barrier. Hyaluronan-degrading enzymes act to degrade hyaluronan by cleaving hyaluronan polymers, which are composed of repeating disaccharides units: D-glucuronic acid (GlcA) and N-acetyl-D-glucosamine (GlcNAc), linked together via alternating β-1→4 and β-1→3 glycosidic bonds. By catalyzing the hydrolysis of hyaluronic acid, a major constituent of the interstitial barrier, hyaluronan-degrading enzymes lower the viscosity of hyaluronic acid, thereby increasing tissue permeability. Accordingly, hyaluronan-degrading enzymes for modification in the methods provided herein include any enzyme having the ability to catalyze the cleavage of a hyaluronan disaccharide chain or polymer. In some examples, the hyaluronan-degrading enzyme cleaves the β-1→4 glycosidic bond in the hyaluronan chain or polymer. In other examples, the hyaluronan-degrading enzyme catalyzes the cleavage of the β-1→3 glycosidic bond in the hyaluronan chain or polymer.

Hyaluronan-degrading enzymes include enzymes that are membrane-bound or that are soluble forms that are secreted from cells. Thus, where hyaluronan-degrading enzymes include a glycosylphosphatidylinositol (GPI) anchor signal sequence and/or are otherwise membrane-anchored or insoluble, such hyaluronan-degrading enzymes can be provided in soluble form by C-terminal truncation or deletion of all or a portion of the GPI anchor signal sequence to render the enzyme secreted and soluble. Thus, hyaluronan-degrading enzymes include C-terminally truncated variants, e.g., truncated to remove all or a portion of a GPI anchor signal sequence. Examples of such soluble hyaluronidases are soluble PH20 hyaluronides, such as any set forth in U.S. Pat. No. 7,767,429; U.S. Publication Nos. US 2004/0268425 and US 2010/0143457.

Exemplary hyaluronan-degrading enzymes are non-human animal or human hyaluronidases, bacterial hyaluronidases, hyaluronidases from leeches or chondroitinases that exhibit hyaluronan-degrading activity, including soluble or truncated forms thereof that are active. Exemplary non-human animal hyaluronidases are any set forth in any of SEQ ID NOS: 8-31, 387-392, 399, 400, 401-416, or mature, C-terminally truncated variants that are soluble and active, or active forms thereof. Exemplary human hyaluronidases are any set forth in any of SEQ ID NOS: 2, 3, 6, 7, 32-66, 68-72 or 417-420, or mature, C-terminally truncated variants that are soluble and active, or active forms thereof, and in particular any of SEQ ID NOS: 3, 7, 32-66, 69 or 72. Exemplary bacterial hyaluronidases are any set forth in any of SEQ ID NOS: 421-451 or mature, C-terminally truncated variants that are soluble and active, or active forms thereof. Exemplary chondroitinases that have hyaluronan-degrading enzyme activity are set forth in SEQ ID NOS:452-454, or mature, C-terminally truncated variants that are soluble and active, or active forms thereof.

Any of such hyaluronan-degrading enzymes can be modified and screened in the methods herein to identify a modified hyaluronan-degrading enzyme that exhibits stability under thermal stress conditions. For example, the modified PH20 polypeptide can be modified compared to an unmodified PH20 polypeptide, such as any known PH20 polypeptide native, wildtype or reference polypeptide. For example, the modified PH20 polypeptide is modified compared to a full-length, soluble or active form of a PH20 polypeptide, such as any set forth in any of SEQ ID NOS: 3, 7, 32-66, 69 or 72, or a polypeptide that exhibits at least 85%, such as at least 86%, 87%, 88%, 89%, 90%, 91%, 92%, 93%, 94%, 95%, 96%, 97%, 98%, 99% or more sequence identity to any of SEQ ID NOS: 3, 7, 32-66, 69 or 72. In particular examples of the method herein, the starting or unmodified PH20 polypeptide has the sequence of amino acids set forth in SEQ ID NO:3.

Libraries or collections of modified hyaluronan-degrading enzymes can be screened. Hyaluronan-degrading enzymes can be modified by any process known to one of skill in the art that can alter the structure of a protein. Examples of modifications include replacement, addition, and deletion of one or more amino acids of the protein to form libraries or collections of modified hyaluronan-degrading enzymes. It is within the level of one of skill in the art to generate modified or variant proteins for use in the methods herein. Methods of mutagenesis are well known in the art and include, for example, site-directed mutagenesis such as for example QuikChange (Stratagene) or saturation mutagenesis. Mutagenesis methods include, but are not limited to, site-mediated mutagenesis, PCR mutagenesis, cassette mutagenesis, site-directed mutagenesis, random point mutagenesis, mutagenesis using uracil containing templates, oligonucleotide-directed mutagenesis, phosphorothioate-modified DNA mutagenesis, mutagenesis using gapped duplex DNA, point mismatch repair, mutagenesis using repair-deficient host strains, restriction-selection and restriction-purification, deletion mutagenesis, mutagenesis by total gene synthesis, double-strand break repair, and many others known to persons of skill. In the methods herein, mutagenesis can be effected across the full length of a protein or within a region of a protein. The mutations can be made rationally or randomly.

In some examples, the methods provided herein are performed such that the identity of each mutant protein is known a priori before the protein is tested. For example, the methods provided herein can be conducive to mutagenesis and screening or testing methods that are addressable. This can permit the ease of comparisons between the activities of tested proteins without the need for sequencing of identified proteins. For example, site-directed mutagenesis methods can be used to individually generate mutant proteins. Mutagenesis can be performed by the replacement of single amino acid residues at specific target positions one-by-one, such that each individual mutant generated is the single product of each single mutagenesis reaction. Mutant DNA molecules can be designed, generated by mutagenesis and cloned individually, such as in addressable arrays, such that they are physically separated from each other and each one is the single product of an independent mutagenesis reaction. The amino acids selected to replace the target positions on the particular protein being optimized can be either all of the remaining 19 amino acids, or a more restricted group containing only selected amino acids. In some methods provided herein, each amino acid that is replaced is independently replaced by 19 of the remaining amino acids or by less than 19 of the remaining amino acids, such as 10, 11, 12, 13, 14, 15, 16, 17 or 18 of the remaining amino acids.

2. Screening or Testing for Activity Under Thermal Stress Conditions

In the method, a modified hyaluronan-degrading enzyme or enzymes is/are tested or screened for hyaluronidase activity under a thermal stress condition. The thermal stress condition need not be a condition or agent that is completely deadly to the enzyme, but generally is a thermal condition that destabilizes enzyme activity over time. The thermal stress condition is one that is chosen because it effects instability or denaturation of the unmodified hyaluronan-degrading enzyme not containing the modification(s). For example, a thermal stress condition is a temperature and incubation time at which the starting or reference hyaluronan-degrading enzyme (i e unmodified hyaluronan-degrading enzyme) loses 50% or more of its activity, 50% or more of its solubility or 50% or more of its secondary or tertiary structure, such as 60%, 70%, 80%, 90%, or more of an activity or property. Such a condition can be empirically determined by a skilled artisan for any starting or reference hyaluronan-degrading enzyme (i e unmodified hyaluronan-degrading enzyme), for example, based on a T₅₀ as determined in a thermal challenge assay or based on the melting temperature (Tm) of the enzyme. For example, the thermal stress condition is a temperature and incubation time at which the starting or reference hyaluronan-degrading enzyme (i e unmodified hyaluronan-degrading enzyme) loses more than 60%, 70%, 80%, 90% or more of its activity, solubility or secondary or tertiary structure.

For example, a thermal challenge assay can be used to assess activity of a hyaluronan-degrading enzyme (i e unmodified hyaluronan-degrading enzyme) across a range of temperatures over a defined time period in order to determine the thermal stress condition. It is understood that the thermal stress condition is a function of time, and that the temperature causing thermal stress is inversely proportional to time. For example, the higher the temperature, the shorter the amount of time that thermal instability is achieved, and the lower the temperature, the longer the amount of time that thermal instability is achieved. The time period chosen can be user selected. The temperature at which 50% of the hyaluronidase activity is retained can be determined and is the T₅₀ or Tc value for the time period, which is an indicator of the stability of the particular protein when incubated at the temperature for the time period. In order to identify variant polypeptides with increased thermal stability or resistance, the T₅₀ value of the unmodified hyaluronan-degrading enzyme can be used as the reference point of thermal stability, whereby modified hyaluronan-degrading enzymes are incubated for the time period at temperatures that are equal to or greater than the T₅₀ value for the time.

In another example, the thermal stress condition can be based on the melting temperature (Tm) of a reference hyaluronan-degrading enzyme (i.e. unmodified hyaluronan-degrading enzyme) using any method that can extrapolate or assess the folded state of the molecule. For example, analytical spectroscopy techniques, such as dynamic light scattering methods, can be used. The temperature at which 50% of molecules are in a folded state can be determined and is the Tm of the particular enzyme, which is an indicator of the stability of the particular protein. In order to identify variant polypeptides with increased thermal stability or resistance, the Tm value of the unmodified hyaluronan-degrading enzyme can be used as the reference point of thermal stability, whereby modified hyaluronan-degrading enzymes are incubated for a predetermined time at temperatures that are equal to or greater than the Tm value for the time.

Thus, in aspects of the method herein, modified hyaluronan-degrading enzyme or enzymes is/are tested or screened for hyaluronidase activity under a thermal stress condition by incubation at a temperature that is equal to or is greater than the T₅₀ or the Tm of the corresponding reference hyaluronan-degrading enzyme (i.e. unmodified hyaluronan-degrading enzyme) for a predetermined time. For example, the modified hyaluronan-degrading enzyme or enzymes is/are tested or screened for hyaluronidase activity under a thermal stress condition that is a temperature that is greater than 1° C., 2° C., 3° C., 4° C., 5° C., 6° C., 7° C., 8° C., 9° C., 10° C., 20° C., 21° C., 22° C., 24° C., 25° C. or more than the T₅₀ or the Tm of the corresponding reference hyaluronan-degrading enzyme (i e unmodified hyaluronan-degrading enzyme) for a predetermined time. The predetermined time can be any time as selected by the end user of the method as described herein below. For example, as shown in the Examples herein, for the exemplary PH20 hyaluronidase designated rHuPH20, which is a soluble C-terminally truncated fragment of human PH20, the Tm is about 44° C. The T₅₀ for 10 minutes is about or less than 49° C. to 52° C.

For example, in practice of the method herein, the thermal stress condition can be one in which the modified hyaluronan-degrading enzyme is incubated at a temperature that is greater than 45° C., and generally greater than 50° C., such as greater than 51° C., 52° C., 53° C., 54° C., 55° C., 56° C., 57° C., 58° C., 59° C., 60° C., 61° C., 62° C., 63° C., 64° C., 65° C. or higher.

The predetermined time of incubation can be user selected. The incubation or exposure can be for any desired length of time, and can be empirically determined by one of skill in the art. As an example, where a T₅₀ value based on a thermal challenge assay of a reference or modified hyaluronan-degrading enzyme is used as the baseline of thermal stability to improve thermal stability, the time period correlating to the T₅₀ value is used (i.e. the time period at which the thermal challenge was performed). For example, the modified hyaluronan-degrading enzyme can be incubated at a desired temperature for or about for 1 minute to 1 month, such as 1 minute to 3 weeks, 1 minute to 2 weeks, 1 minute to 1 week, 1 minute to 24 hours, 1 minute to 12 hours, such as 5 minutes to 30 minutes, 5 minutes to 15 minutes, 30 minutes to 6 hours or 1 hour to 4 hours, and generally at least or about at least 30 minutes, 1 hour, 2 hours, 3 hours, 4 hours, 5 hours, 6 hours, 7 hours, 8 hours, 9 hours, 10 hours, 11 hours or 12 hours. For purposes of performing a high-throughput assay or otherwise rapidly screening candidates, the predetermined time is selected that is less than 2 hours, and generally less than 1 hour, 30 minutes, 20 minutes, 10 minutes of less. For example, screening is performed after incubation at the temperature for 10 minutes. After the incubation for the predetermined time, the sample is returned to a thermal neutral condition in order to remove the polypeptide from further destabilizing conditions.

In the methods, the modified hyaluronan degrading enzyme also is tested or screened for hyaluronidase activity under a thermal neutral condition at which the starting or reference hyaluronan-degrading enzyme (i e unmodified hyaluronan-degrading enzyme) retains or maintains activity. For example, the modified hyaluronan-degrading enzyme is incubated at a temperature of 2° C. to 8° C., such as 4° C., for the predetermined time and then hyaluronidase activity determined. For comparison, the predetermined time is the same as tested in the thermal stress condition.

Hence, each member of a library or collection of modified hyaluronan-degrading enzymes is incubated under or exposed to a thermal stress condition, such as any described above. The same modified enzyme also is incubated or exposed to a thermal neutral condition, such as any described above. The incubation or exposure can occur in vivo or in vitro. Typically, the assay is performed in vitro. The activities under both conditions are compared in order to identify a modified hyaluronan-degrading enzymes that exhibit stability upon exposure to the thermal stress condition. Further, in screening or identifying the activity of the enzyme under the two different sets of conditions, generally the only conditions that are varied in the assay relate to the temperature. The other conditions of the assay, including but not limited to, time and/or other incubation conditions, can be the same for both sets of conditions. In any examples where a modified hyaluronan-degrading enzyme is assessed, it is understood that an unmodified hyaluronan-degrading enzyme not containing the modifications(s) also can be assessed under similar assay conditions for comparison.

For example, in aspects of the method herein, a modified hyaluronan-degrading enzyme or enzymes is/are tested or screened for hyaluronidase activity after incubation at 52° C. for 10 minutes, and also tested or screened for hyaluronidase activity after incubation at 4° C. for 10 minutes. Each hyaluronan-degrading enzyme can be a member of a collection of modified hyaluronan-degrading enzymes. Each hyaluronan-degrading enzyme can be tested separately under each condition from the other hyaluronan-degrading enzymes (e.g. modified hyaluronan-degrading enzymes, such as modified PH20 polypeptides) in the collection.

After the time of incubation or exposure, the sample or composition containing the modified hyaluronan-degrading enzyme (or control unmodified enzyme) is assessed for hyaluronidase assay. Assays to assess hyaluronidase activity are well known in the art. Examples of such assays are described in Section G. In one example, hyaluronidase activity can be assessed in a microturbidity assay, wherein the amount of undegraded HA is measured by the addition of a reagent that precipitates HA (e.g., Cetylpyridinium chloride (CPC) or acidified serum) after the enzyme is allowed to react with HA. In another example, hyaluronidase activity can be assessed using a microtiter assay in which residual biotinylated hyaluronic acid is measured following incubation with hyaluronidase (see e.g., Frost and Stern (1997) Anal. Biochem. 251:263-269, U.S. Pat. Publication No. 20050260186). The resulting activities under each of the tested conditions is determined and compared.

3. Selection or Identification

After testing, the hyaluronidase activity is assessed in order to identify modified hyaluronan-degrading enzymes that, after incubation at the thermal stress condition (e.g. incubation at 52° C. for 10 minutes), exhibit greater than or at least 50% of the activity achieved after incubation at the thermal neutral condition (e.g. incubation 4° C. for 10 minutes). The desired level or amount of activity selected as a cut-off in the methods can be empirically determined by the user, and is dependent on factors such as the particular hyaluronan-degrading enzyme, the desired application or use of the hyaluronan-degrading enzyme, the particular temperature condition and other similar factors. Typically, a modified hyaluronan-degrading enzyme is identified that exhibits at least 55%, 60%, 65%, 70%, 75%, 80%, 85%, 90%, 95% or more of the activity after incubation under a thermal stress condition compared to after incubation under a thermal neutral condition.

Additionally or alternatively, the activity of the modified hyaluronan-degrading enzyme exposed to a thermal stress condition is compared to the activity of the corresponding unmodified hyaluronan-degrading enzyme that is exposed to the same thermal stress condition. In such examples, it is understood that the activity of the modified and unmodified enzyme are tested under the same conditions (e.g., time, temperature, composition), except for the difference in the particular enzyme tested (unmodified versus modified). A modified hyaluronan-degrading enzyme is identified that exhibits greater activity, such as at least 110%, 120%, 130%, 140%, 150%, 160%, 170%, 180%, 190%, 200%, 250%, 300%, 400%, 500% or more of the activity of the unmodified hyaluronan-degrading enzyme.

4. Iterative Methods

The method provided herein also is iterative. In one example, after the method is performed, any modified hyaluronan-degrading enzymes identified as exhibiting thermal stability, such as increased thermal resistance, is modified or further modified to increase or optimize the stability. A secondary library can be created by introducing additional modifications in a first identified modified hyaluronan-degrading enzyme. For example, modifications that were identified as conferring stability, such as increasing stability, can be combined to generate a combinatorial library. The secondary library can be tested using the assays and methods described herein.

In another example of an iterative aspect of the method, modified hyaluronan-degrading enzymes that are identified as not exhibiting stability such as increased stability (e.g., such that they are not active or do not have increased activity under the a thermal stress condition), can be further modified and retested for stability under a thermal stress condition. The further modifications can be targeted near particular regions (e.g., particular amino acid residues) associated with activity and/or stability of the molecule. For example, residues that are associated with activity and/or stability of the molecule generally are critical residues that are involved in the structural folding or other activities of the molecule. Hence, such residues are required for activity, generally under any condition. Critical residues can be identified because, when mutated, a normal activity of the protein is ablated or reduced. For example, critical residues can be identified that, when mutated in a hyaluronan-degrading enzyme, exhibit reduced or ablated hyaluronidase activity under a normal or control assay condition. A further library of modified proteins can be generated with amino acid mutations targeted at or near to the identified critical amino acid residues, such as adjacent to the identified critical amino acid residues. In some examples, the mutations can be amino acid replacement to any other of up to 19 other amino acid residues. The secondary library can be tested using the assays and methods described herein.

### E. PRODUCTION OF MODIFIED PH20 POLYPEPTIDES AND ENCODING NUCLEIC ACID MOLECULES

Polypeptides of a modified PH20 polypeptide set forth herein can be obtained by methods well known in the art for protein purification and recombinant protein expression. Polypeptides also can be synthesized chemically. Modified or variant, including truncated, forms can be engineered from a wildtype polypeptide using standard recombinant DNA methods. For example, modified PH20 polypeptides can be engineered from a wildtype polypeptide, such as by site-directed mutagenesis.

1. Isolation or Preparation of Nucleic Acids Encoding PH20 Polypeptides

Polypeptides can be cloned or isolated using any available methods known in the art for cloning and isolating nucleic acid molecules. Such methods include PCR amplification of nucleic acids and screening of libraries, including nucleic acid hybridization screening, antibody-based screening and activity-based screening.

For example, when the polypeptides are produced by recombinant means, any method known to those of skill in the art for identification of nucleic acids that encode desired genes can be used. Any method available in the art can be used to obtain a full length or partial (i.e., encompassing the entire coding region) cDNA or genomic DNA clone encoding a PH20, such as from a cell or tissue source.

Methods for amplification of nucleic acids can be used to isolate nucleic acid molecules encoding a desired polypeptide, including for example, polymerase chain reaction (PCR) methods. Examples of such methods include use of a Perkin-Elmer Cetus thermal cycler and Taq polymerase (Gene Amp). A nucleic acid containing material can be used as a starting material from which a desired polypeptide-encoding nucleic acid molecule can be isolated. For example, DNA and mRNA preparations, cell extracts, tissue extracts, fluid samples (e.g., blood, serum, saliva), samples from healthy and/or diseased subjects can be used in amplification methods. The source can be from any eukaryotic species including, but not limited to, vertebrate, mammalian, human, porcine, bovine, feline, avian, equine, canine, and other primate sources. Nucleic acid libraries also can be used as a source of starting material. Primers can be designed to amplify a desired polypeptide. For example, primers can be designed based on expressed sequences from which a desired polypeptide is generated. Primers can be designed based on back-translation of a polypeptide amino acid sequence. If desired, degenerate primers can be used for amplification. Oligonucleotide primers that hybridize to sequences at the 3′ and 5′ termini of the desired sequence can be uses as primers to amplify by PCR sequences from a nucleic acid sample. Primers can be used to amplify the entire full-length PH20, or a truncated sequence thereof, such as a nucleic acid encoding any of the soluble PH20 polypeptides provided herein. Nucleic acid molecules generated by amplification can be sequenced and confirmed to encode a desired polypeptide.

Additional nucleotide sequences can be joined to a polypeptide-encoding nucleic acid molecule, including linker sequences containing restriction endonuclease sites for the purpose of cloning the synthetic gene into a vector, for example, a protein expression vector or a vector designed for the amplification of the core protein coding DNA sequences. Furthermore, additional nucleotide sequences specifying functional DNA elements can be operatively linked to a polypeptide-encoding nucleic acid molecule. Examples of such sequences include, but are not limited to, promoter sequences designed to facilitate intracellular protein expression, and secretion sequences, for example heterologous signal sequences, designed to facilitate protein secretion. Such sequences are known to those of skill in the art. For example, exemplary heterologous signal sequences include, but are not limited to, human and mouse kappa IgG heterologous signal sequences set forth in SEQ ID NO: 398. Additional nucleotide residue sequences such as sequences of bases specifying protein binding regions also can be linked to enzyme-encoding nucleic acid molecules. Such regions include, but are not limited to, sequences of residues that facilitate or encode proteins that facilitate uptake of an enzyme into specific target cells, or otherwise alter pharmacokinetics of a product of a synthetic gene.

In addition, tags or other moieties can be added, for example, to aid in detection or affinity purification of the polypeptide. For example, additional nucleotide residue sequences such as sequences of bases specifying an epitope tag or other detectable marker also can be linked to enzyme-encoding nucleic acid molecules. Examples of such sequences include nucleic acid sequences encoding a His tag or Flag Tag.

The identified and isolated nucleic acids can then be inserted into an appropriate cloning vector. A large number of vector-host systems known in the art can be used. Possible vectors include, but are not limited to, plasmids or modified viruses, but the vector system must be compatible with the host cell used. Such vectors include, but are not limited to, bacteriophages such as lambda derivatives, or plasmids such as pCMV4, pBR322 or pUC plasmid derivatives or the Bluescript vector (Stratagene, La Jolla, Calif.). Other expression vectors include the HZ24 expression vector exemplified herein (see e.g., SEQ ID NOS:4 and 5). The insertion into a cloning vector can, for example, be accomplished by ligating the DNA fragment into a cloning vector which has complementary cohesive termini. Insertion can be effected using TOPO cloning vectors (Invitrogen, Carlsbad, Calif.).

If the complementary restriction sites used to fragment the DNA are not present in the cloning vector, the ends of the DNA molecules can be enzymatically modified. Alternatively, any site desired can be produced by ligating nucleotide sequences (linkers) onto the DNA termini; these ligated linkers can contain specific chemically synthesized oligonucleotides encoding restriction endonuclease recognition sequences. In an alternative method, the cleaved vector and protein gene can be modified by homopolymeric tailing.

Recombinant molecules can be introduced into host cells via, for example, transformation, transfection, infection, electroporation and sonoporation, so that many copies of the gene sequence are generated. In specific embodiments, transformation of host cells with recombinant DNA molecules that incorporate the isolated protein gene, cDNA, or synthesized DNA sequence enables generation of multiple copies of the gene. Thus, the gene can be obtained in large quantities by growing transformants, isolating the recombinant DNA molecules from the transformants and, when necessary, retrieving the inserted gene from the isolated recombinant DNA.

In addition to recombinant production, modified PH20 polypeptides provided herein can be produced by direct peptide synthesis using solid-phase techniques (see e.g., Stewart et al. (1969) Solid-Phase Peptide Synthesis, WH Freeman Co., San Francisco; Merrifield J (1963) J Am Chem Soc., 85:2149-2154). In vitro protein synthesis can be performed using manual techniques or by automation. Automated synthesis can be achieved, for example, using Applied Biosystems 431A Peptide Synthesizer (Perkin Elmer, Foster City Calif.) in accordance with the instructions provided by the manufacturer. Various fragments of a polypeptide can be chemically synthesized separately and combined using chemical methods.

2. Generation of Mutant or Modified Nucleic Acid and Encoding Polypeptides

The modifications provided herein can be made by standard recombinant DNA techniques such as are routine to one of skill in the art. Any method known in the art to effect mutation of any one or more amino acids in a target protein can be employed. Methods include standard site-directed mutagenesis (using e.g., a kit, such as QuikChange available from Stratagene) of encoding nucleic acid molecules, or by solid phase polypeptide synthesis methods.

3. Vectors and Cells

For recombinant expression of one or more of the desired proteins, such as any modified PH20 polypeptide described herein, the nucleic acid containing all or a portion of the nucleotide sequence encoding the protein can be inserted into an appropriate expression vector, i.e., a vector that contains the necessary elements for the transcription and translation of the inserted protein coding sequence. The necessary transcriptional and translational signals also can be supplied by the native promoter for enzyme genes, and/or their flanking regions.

Also provided are vectors that contain a nucleic acid encoding the enzyme. Cells containing the vectors also are provided. The cells include eukaryotic and prokaryotic cells, and the vectors are any suitable for use therein. Generally, the cell is a cell that is capable of effecting glyosylation of the encoded protein.

Prokaryotic and eukaryotic cells containing the vectors are provided. Such cells include bacterial cells, yeast cells, fungal cells, Archea, plant cells, insect cells and animal cells. The cells are used to produce a protein thereof by growing the above-described cells under conditions whereby the encoded protein is expressed by the cell, and recovering the expressed protein. For purposes herein, for example, the enzyme can be secreted into the medium.

A host cell strain can be chosen for its ability to modulate the expression of the inserted sequences or to process the expressed protein in the desired fashion. Such modifications of the polypeptide include, but are not limited to, acetylation, carboxylation, glycosylation, phosphorylation, lipidation and acylation. Post-translational processing can impact the folding and/or function of the polypeptide. Different host cells, such as, but not limited to, CHO (DG44, DXB11, CHO-K1), HeLa, MCDK, 293 and WI38 have specific cellular machinery and characteristic mechanisms for such post-translational activities and can be chosen to ensure the correct modification and processing of the introduced protein. Generally, the choice of cell is one that is capable of introducing N-linked glycosylation into the expressed polypeptide. Hence, eukaryotic cells containing the vectors are provided. Exemplary eukaryotic cells are mammalian Chinese Hamster Ovary (CHO) cells. For example, CHO cells deficient in dihydrofolate reductase (e.g., DG44 cells) are used to produce polypeptides provided herein. Note that bacterial expression of an PH20 polypeptide provided herein will not result in a catalytically active polypeptide, but when combined with proper glycosylation machinery, the PH20 can be artificially glycosylated.

Provided are vectors that contain a sequence of nucleotides that encodes the modified PH20 polypeptide, coupled to the native or heterologous signal sequence, as well as multiple copies thereof. The vectors can be selected for expression of the enzyme protein in the cell or such that the enzyme protein is expressed as a secreted protein.

A variety of host-vector systems can be used to express the protein encoding sequence. These include but are not limited to mammalian cell systems infected with virus (e.g., vaccinia virus, adenovirus and other viruses); insect cell systems infected with virus (e.g., baculovirus); microorganisms such as yeast containing yeast vectors; or bacteria transformed with bacteriophage, DNA, plasmid DNA, or cosmid DNA. The expression elements of vectors vary in their strengths and specificities. Depending on the host-vector system used, any one of a number of suitable transcription and translation elements can be used.

Any methods known to those of skill in the art for the insertion of DNA fragments into a vector can be used to construct expression vectors containing a chimeric gene containing appropriate transcriptional/translational control signals and protein coding sequences. These methods can include in vitro recombinant DNA and synthetic techniques and in vivo recombinants (genetic recombination). Expression of nucleic acid sequences encoding protein, or domains, derivatives, fragments or homologs thereof, can be regulated by a second nucleic acid sequence so that the genes or fragments thereof are expressed in a host transformed with the recombinant DNA molecule(s). For example, expression of the proteins can be controlled by any promoter/enhancer known in the art. In a specific embodiment, the promoter is not native to the genes for a desired protein. Promoters which can be used include, but are not limited to, the SV40 early promoter (Bernoist and Chambon, Nature 290:304-310 (1981)), the promoter contained in the 3′ long terminal repeat of Rous sarcoma virus (Yamamoto et al. Cell 22:787-797 (1980)), the herpes thymidine kinase promoter (Wagner et al., Proc. Natl. Acad. Sci. USA 78:1441-1445 (1981)), the regulatory sequences of the metallothionein gene (Brinster et al., Nature 296:39-42 (1982)); prokaryotic expression vector promoters, such as the β-lactamase promoter (Jay et al., (1981) Proc. Natl. Acad. Sci. USA 78:5543) or the tac promoter (DeBoer et al., Proc. Natl. Acad. Sci. USA 80:21-25 (1983); see also Gilbert and Villa-Komaroff, “Useful Proteins from Recombinant Bacteria,” Scientific American 242:74-94 (1980)); plant expression vector promoters, such as the nopaline synthetase promoter (Herrera-Estrella et al., Nature 303:209-213 (1983)) or the cauliflower mosaic virus 35S RNA promoter (Gardner et al., Nucleic Acids Res. 9:2871 (1981)), and the promoter of the photosynthetic enzyme ribulose bisphosphate carboxylase (Herrera-Estrella et al., Nature 310:115-120 (1984)); promoter elements from yeast and other fungi such as the Gal4 promoter, the alcohol dehydrogenase promoter, the phosphoglycerol kinase promoter, the alkaline phosphatase promoter, and the following animal transcriptional control regions that exhibit tissue specificity and have been used in transgenic animals: elastase I gene control region which is active in pancreatic acinar cells (Swift et al., Cell 38:639-646 (1984); Ornitz et al., Cold Spring Harbor Symp. Quant. Biol. 50:399-409 (1986); MacDonald, Hepatology 7:425-515 (1987)); insulin gene control region which is active in pancreatic beta cells (Hanahan et al., Nature 315:115-122 (1985)), immunoglobulin gene control region which is active in lymphoid cells (Grosschedl et al., Cell 38:647-658 (1984); Adams et al., Nature 318:533-538 (1985); Alexander et al., Mol. Cell Biol. 7:1436-1444 (1987)), mouse mammary tumor virus control region which is active in testicular, breast, lymphoid and mast cells (Leder et al., Cell 45:485-495 (1986)), albumin gene control region which is active in liver (Pinkert et al., Genes and Devel. 1:268-276 (1987)), alpha-fetoprotein gene control region which is active in liver (Krumlauf et al., Mol. Cell. Biol. 5:1639-1648 (1985); Hammer et al., Science 235:53-58 1987)), alpha-1 antitrypsin gene control region which is active in liver (Kelsey et al., Genes and Devel. 1:161-171 (1987)), beta globin gene control region which is active in myeloid cells (Magram et al., Nature 315:338-340 (1985); Kollias et al., Cell 46:89-94 (1986)), myelin basic protein gene control region which is active in oligodendrocyte cells of the brain (Readhead et al., Cell 48:703-712 (1987)), myosin light chain-2 gene control region which is active in skeletal muscle (Shani, Nature 314:283-286 (1985)), and gonadotrophic releasing hormone gene control region which is active in gonadotrophs of the hypothalamus (Mason et al., Science 234:1372-1378 (1986)).

In a specific embodiment, a vector is used that contains a promoter operably linked to nucleic acids encoding a desired protein, or a domain, fragment, derivative or homolog thereof, one or more origins of replication, and optionally, one or more selectable markers (e.g., an antibiotic resistance gene). Depending on the expression system, specific initiation signals also are required for efficient translation of a PH20 sequence. These signals include the ATG initiation codon and adjacent sequences. In cases where the initiation codon and upstream sequences of PH20 or soluble forms thereof are inserted into the appropriate expression vector, no additional translational control signals are needed. In cases where only a coding sequence, or a portion thereof, is inserted, exogenous transcriptional control signals including the ATG initiation codon must be provided. Furthermore, the initiation codon must be in the correct reading frame to ensure transcription of the entire insert. Exogenous transcriptional elements and initiation codons can be of various origins, both natural and synthetic. The efficiency of expression can be enhanced by the inclusion of enhancers appropriate to the cell system in use (Scharf et al. (1994) Results Probl Cell Differ 20:125-62; Bittner et al. (1987) Methods in Enzymol, 153:516-544).

Exemplary plasmid vectors for transformation of E. coli cells include, for example, the pQE expression vectors (available from Qiagen, Valencia, Calif.; see also literature published by Qiagen describing the system). pQE vectors have a phage T5 promoter (recognized by E. coli RNA polymerase) and a double lac operator repression module to provide tightly regulated, high-level expression of recombinant proteins in E. coli, a synthetic ribosomal binding site (RBS II) for efficient translation, a 6×His tag coding sequence, t₀ and T1 transcriptional terminators, ColE1 origin of replication, and a beta-lactamase gene for conferring ampicillin resistance. The pQE vectors enable placement of a 6×His tag at either the N- or C-terminus of the recombinant protein. Such plasmids include pQE 32, pQE 30, and pQE 31 which provide multiple cloning sites for all three reading frames and provide for the expression of N-terminally 6×His-tagged proteins. Other exemplary plasmid vectors for transformation of E. coli cells, include, for example, the pET expression vectors (see, U.S. Pat. No. 4,952,496; available from Novagen, Madison, Wis.; see, also literature published by Novagen describing the system). Such plasmids include pET 11a, which contains the T7lac promoter, T7 terminator, the inducible E. coli lac operator, and the lac repressor gene; pET 12a-c, which contains the T7 promoter, T7 terminator, and the E. coli ompT secretion signal; and pET 15b and pET19b (Novagen, Madison, Wis.), which contain a His-Tag™ leader sequence for use in purification with a His column and a thrombin cleavage site that permits cleavage following purification over the column, the T7-lac promoter region and the T7 terminator.

Typically, vectors can be plasmids, viral vectors, or others known in the art, used for expression of the modified PH20 polypeptide in vivo or in vitro. For example, the modified PH20 polypeptide is expressed in mammalian cells, including, for example, Chinese Hamster Ovary (CHO) cells. An exemplary vector for mammalian cell expression is the HZ24 expression vector. The HZ24 expression vector was derived from the pCI vector backbone (Promega). It contains DNA encoding the Beta-lactamase resistance gene (AmpR), an F1 origin of replication, a Cytomegalovirus immediate-early enhancer/promoter region (CMV), and an SV40 late polyadenylation signal (SV40). The expression vector also has an internal ribosome entry site (IRES) from the ECMV virus (Clontech) and the mouse dihydrofolate reductase (DHFR) gene.

Viral vectors, such as adenovirus, retrovirus or vaccinia virus vectors, can be employed. In some examples, the vector is a defective or attenuated retroviral or other viral vector (see U.S. Pat. No. 4,980,286). For example, a retroviral vector can be used (see Miller et al., Meth. Enzymol. 217: 581-599 (1993)). These retroviral vectors have been modified to delete retroviral sequences that are not necessary for packaging of the viral genome and integration into host cell DNA.

In some examples, viruses armed with a nucleic acid encoding a modified PH20 polypeptide can facilitate their replication and spread within a target tissue for example. The target tissue can be a cancerous tissue whereby the virus is capable of selective replication within the tumor. The virus can also be a non-lytic virus wherein the virus selectively replicates under a tissue specific promoter. As the viruses replicate, the coexpression of the PH20 polypeptide with viral genes will facilitate the spread of the virus in vivo.

4. Expression

Modified PH20 polypeptides can be produced by any method known to those of skill in the art including in vivo and in vitro methods. Desired proteins can be expressed in any organism suitable to produce the required amounts and forms of the proteins, such as for example, those needed for administration and treatment. Expression hosts include prokaryotic and eukaryotic organisms such as E. coli, yeast, plants, insect cells, mammalian cells, including human cell lines and transgenic animals. Expression hosts can differ in their protein production levels as well as the types of post-translational modifications that are present on the expressed proteins. The choice of expression host can be made based on these and other factors, such as regulatory and safety considerations, production costs and the need and methods for purification.

Many expression vectors are available and known to those of skill in the art and can be used for expression of proteins. The choice of expression vector will be influenced by the choice of host expression system. In general, expression vectors can include transcriptional promoters and optionally enhancers, translational signals, and transcriptional and translational termination signals. Expression vectors that are used for stable transformation typically have a selectable marker which allows selection and maintenance of the transformed cells. In some cases, an origin of replication can be used to amplify the copy number of the vector.

Modified PH20 polypeptides also can be utilized or expressed as protein fusions. For example, an enzyme fusion can be generated to add additional functionality to an enzyme. Examples of enzyme fusion proteins include, but are not limited to, fusions of a signal sequence, a tag such as for localization, e.g., a 6×His or His₆ tag or a myc tag, or a tag for purification, for example, a GST fusion, and a sequence for directing protein secretion and/or membrane association.

For long-term, high-yield production of recombinant proteins, stable expression is desired. For example, cell lines that stably express a modified PH20 polypeptide can be transformed using expression vectors that contain viral origins of replication or endogenous expression elements and a selectable marker gene. Following the introduction of the vector, cells can be allowed to grow for 1-2 days in an enriched medium before they are switched to selective media. The purpose of the selectable marker is to confer resistance to selection, and its presence allows growth and recovery of cells that successfully express the introduced sequences. Resistant cells of stably transformed cells can be proliferated using tissue culture techniques appropriate to the cell types.

Any number of selection systems can be used to recover transformed cell lines. These include, but are not limited to, the herpes simplex virus thymidine kinase (Wigler, M et al. (1977) Cell, 11:223-32) and adenine phosphoribosyltransferase (Lowy, I et al. (1980) Cell, 22:817-23) genes, which can be employed in TK- or APRT-cells, respectively. Also, antimetabolite, antibiotic or herbicide resistance can be used as the basis for selection. For example, DHFR, which confers resistance to methotrexate (Wigler, M et al. (1980) Proc. Natl. Acad. Sci, 77:3567-70); npt, which confers resistance to the aminoglycosides neomycin and G-418 (Colbere-Garapin, F et al. (1981) J. Mol. Biol., 150:1-14); and als or pat, which confer resistance to chlorsulfuron and phosphinotricin acetyltransferase, respectively, can be used. Additional selectable genes have been described, for example, trpB, which allows cells to utilize indole in place of typtophan or hisD, which allows cells to utilize histinol in place of histidine (Hartman S C and R C Mulligan (1988) Proc. Natl. Acad. Sci, 85:8047-51). Visible markers, such as but not limited to, anthocyanins, beta glucuronidase and its substrate, GUS, and luciferase and its substrate luciferin, also can be used to identify transformants and also to quantify the amount of transient or stable protein expression attributable to a particular vector system (Rhodes C A et al. (1995) Methods Mol. Biol. 55:121-131).

The presence and expression of PH20 polypeptides can be monitored. For example, detection of a functional polypeptide can be determined by testing the conditioned media for hyaluronidase enzyme activity under appropriate conditions. Exemplary assays to assess the solubility and activity of expressed proteins are provided herein.

a. Prokaryotic Cells

Prokaryotes, especially E. coli, provide a system for producing large amounts of proteins. Transformation of E. coli is a simple and rapid technique well known to those of skill in the art. Expression vectors for E. coli can contain inducible promoters. Such promoters are useful for inducing high levels of protein expression and for expressing proteins that exhibit some toxicity to the host cells. Examples of inducible promoters include the lac promoter, the trp promoter, the hybrid tac promoter, the T7 and SP6 RNA promoters and the temperature regulated λPL promoter.

Proteins, such as any provided herein, can be expressed in the cytoplasmic environment of E. coli. The cytoplasm is a reducing environment, and for some molecules, this can result in the formation of insoluble inclusion bodies. Reducing agents such as dithiothreotol and β-mercaptoethanol and denaturants, such as guanidine-HCl and urea can be used to resolubilize the proteins. An alternative approach effects protein expression in the periplasmic space of bacteria which provides an oxidizing environment and chaperonin-like and disulfide isomerases, which can aid in the production of soluble protein. Typically, a leader sequence is fused to the protein to be expressed which directs the protein to the periplasm. The leader is then removed by signal peptidases inside the periplasm. Examples of periplasmic-targeting leader sequences include the pelB leader from the pectate lyase gene and the leader derived from the alkaline phosphatase gene. In some cases, periplasmic expression allows leakage of the expressed protein into the culture medium. The secretion of proteins allows quick and simple purification from the culture supernatant. Proteins that are not secreted can be obtained from the periplasm by osmotic lysis. Similar to cytoplasmic expression, in some cases proteins can become insoluble and denaturants and reducing agents can be used to facilitate solubilization and refolding. Temperature of induction and growth also can influence expression levels and solubility, typically temperatures between 25° C. and 37° C. are used. Typically, bacteria produce aglycosylated proteins. Thus, if proteins require glycosylation for function, glycosylation can be added in vitro after purification from host cells.

b. Yeast Cells

Yeasts such as Saccharomyces cerevisae, Schizosaccharomyces pombe, Yarrowia lipolytica, Kluyveromyces lactis and Pichia pastoris are well known yeast expression hosts that can be used for production of proteins, such as any described herein. Yeast can be transformed with episomal replicating vectors or by stable chromosomal integration by homologous recombination. Typically, inducible promoters are used to regulate gene expression. Examples of such promoters include GAL1, GAL7 and GAL5 and metallothionein promoters, such as CUP1, AOX1 or other Pichia or other yeast promoters. Expression vectors often include a selectable marker such as LEU2, TRP1, HIS3 and URA3 for selection and maintenance of the transformed DNA. Proteins expressed in yeast are often soluble. Co-expression with chaperonins such as Bip and protein disulfide isomerase can improve expression levels and solubility. Additionally, proteins expressed in yeast can be directed for secretion using secretion signal peptide fusions such as the yeast mating type alpha-factor secretion signal from Saccharomyces cerevisae and fusions with yeast cell surface proteins such as the Aga2p mating adhesion receptor or the Arxula adeninivorans glucoamylase. A protease cleavage site such as for the Kex-2 protease, can be engineered to remove the fused sequences from the expressed polypeptides as they exit the secretion pathway. Yeast also is capable of glycosylation at Asn-X-Ser/Thr motifs.

c. Insects and Insect Cells

Insect cells, particularly using baculovirus expression, are useful for expressing polypeptides such as PH20 polypeptides. Insect cells express high levels of protein and are capable of most of the post-translational modifications used by higher eukaryotes. Baculoviruses have a restrictive host range which improves the safety and reduces regulatory concerns of eukaryotic expression. Typical expression vectors use a promoter for high level expression such as the polyhedrin promoter of baculovirus. Commonly used baculovirus systems include a baculovirus, such as the Autographa californica nuclear polyhedrosis virus (AcNPV) or the Bombyx mori nuclear polyhedrosis virus (BmNPV), and an insect cell line, such as Sf9 derived from Spodoptera frugiperda, Pseudaletia unipuncta (A7S) and Danaus plexippus (DpN1). For high-level expression, the nucleotide sequence of the molecule to be expressed is fused immediately downstream of the polyhedrin initiation codon of the virus. Mammalian secretion signals are accurately processed in insect cells and can be used to secrete the expressed protein into the culture medium. In addition, the cell lines Pseudaletia unipuncta (A7S) and Danaus plexippus (DpN1) produce proteins with glycosylation patterns similar to mammalian cell systems. Exemplary insect cells are those that have been altered to reduce immunogenicity, including those with “mammalianized” baculovirus expression vectors and those lacking the enzyme FT3.

An alternative expression system in insect cells employs stably transformed cells. Cell lines such as the Schnieder 2 (S2) and Kc cells (Drosophila melanogaster) and C7 cells (Aedes albopictus) can be used for expression. The Drosophila metallothionein promoter can be used to induce high levels of expression in the presence of heavy metal induction with cadmium or copper. Expression vectors are typically maintained by the use of selectable markers such as neomycin and hygromycin.

D. Mammalian Expression

Mammalian expression systems can be used to express proteins including PH20 polypeptides. Expression constructs can be transferred to mammalian cells by viral infection such as by adenovirus or by direct DNA transfer such as liposomes, calcium phosphate, DEAE-dextran and by physical means such as electroporation and microinjection. Expression vectors for mammalian cells typically include an mRNA cap site, a TATA box, a translational initiation sequence (Kozak consensus sequence) and polyadenylation elements. IRES elements also can be added to permit bicistronic expression with another gene, such as a selectable marker. Such vectors often include transcriptional promoter-enhancers for high-level expression, for example the SV40 promoter-enhancer, the human cytomegalovirus (CMV) promoter and the long terminal repeat of Rous sarcoma virus (RSV). These promoter-enhancers are active in many cell types. Tissue and cell-type promoters and enhancer regions also can be used for expression. Exemplary promoter/enhancer regions include, but are not limited to, those from genes such as elastase I, insulin, immunoglobulin, mouse mammary tumor virus, albumin, alpha fetoprotein, alpha 1 antitrypsin, beta globin, myelin basic protein, myosin light chain 2, and gonadotropic releasing hormone gene control. Selectable markers can be used to select for and maintain cells with the expression construct. Examples of selectable marker genes include, but are not limited to, hygromycin B phosphotransferase, adenosine deaminase, xanthine-guanine phosphoribosyl transferase, aminoglycoside phosphotransferase, dihydrofolate reductase (DHFR) and thymidine kinase. For example, expression can be performed in the presence of methotrexate to select for only those cells expressing the DHFR gene. Fusion with cell surface signaling molecules such as TCR-ζ and Fc_{ε}RI-γ can direct expression of the proteins in an active state on the cell surface.

Many cell lines are available for mammalian expression including mouse, rat human, monkey, chicken and hamster cells. Exemplary cell lines include but are not limited to CHO, Balb/3T3, HeLa, MT2, mouse NS0 (nonsecreting) and other myeloma cell lines, hybridoma and heterohybridoma cell lines, lymphocytes, fibroblasts, Sp2/0, COS, NIH3T3, HEK293, 293S, 2B8, and HKB cells. Cell lines also are available adapted to serum-free media which facilitates purification of secreted proteins from the cell culture media. Examples include CHO-S cells (Invitrogen, Carlsbad, Calif., cat #11619-012) and the serum free EBNA-1 cell line (Pham et al., (2003) Biotechnol. Bioeng. 84:332-42.). Cell lines also are available that are adapted to grow in special mediums optimized for maximal expression. For example, DG44 CHO cells are adapted to grow in suspension culture in a chemically defined, animal product-free medium.

e. Plants

Transgenic plant cells and plants can be used to express proteins such as any described herein. Expression constructs are typically transferred to plants using direct DNA transfer such as microprojectile bombardment and PEG-mediated transfer into protoplasts, and with agrobacterium-mediated transformation. Expression vectors can include promoter and enhancer sequences, transcriptional termination elements and translational control elements. Expression vectors and transformation techniques are usually divided between dicot hosts, such as Arabidopsis and tobacco, and monocot hosts, such as corn and rice. Examples of plant promoters used for expression include the cauliflower mosaic virus promoter, the nopaline syntase promoter, the ribose bisphosphate carboxylase promoter and the ubiquitin and UBQ3 promoters. Selectable markers such as hygromycin, phosphomannose isomerase and neomycin phosphotransferase are often used to facilitate selection and maintenance of transformed cells. Transformed plant cells can be maintained in culture as cells, aggregates (callus tissue) or regenerated into whole plants. Transgenic plant cells also can include algae engineered to produce hyaluronidase polypeptides. Because plants have different glycosylation patterns than mammalian cells, this can influence the choice of protein produced in these hosts.

5. Purification

Host cells transformed with a nucleic acid sequence encoding a modified PH20 polypeptide can be cultured under conditions suitable for the expression and recovery of the encoded protein from cell culture. The protein produced by a recombinant cell is generally secreted, but may be contained intracellularly depending on the sequence and/or the vector used. As will be understood by those of skill in the art, expression vectors containing nucleic acid encoding PH20 can be designed with signal sequences that facilitate direct secretion of PH20 through prokaryotic or eukaryotic cell membranes.

Thus, methods for purification of polypeptides from host cells will depend on the chosen host cells and expression systems. For secreted molecules, proteins are generally purified from the culture media after removing the cells. For intracellular expression, cells can be lysed and the proteins purified from the extract. When transgenic organisms such as transgenic plants and animals are used for expression, tissues or organs can be used as starting material to make a lysed cell extract. Additionally, transgenic animal production can include the production of polypeptides in milk or eggs, which can be collected, and if necessary, the proteins can be extracted and further purified using standard methods in the art.

Proteins, such as modified PH20 polypeptides, can be purified using standard protein purification techniques known in the art including but not limited to, SDS-PAGE, size fractionation and size exclusion chromatography, ammonium sulfate precipitation and ionic exchange chromatography, such as anion exchange chromatography. Affinity purification techniques also can be utilized to improve the efficiency and purity of the preparations. For example, antibodies, receptors and other molecules that bind PH20 hyaluronidase enzymes can be used in affinity purification. For example, soluble PH20 can be purified from conditioned media.

Expression constructs also can be engineered to add an affinity tag to a protein such as a myc epitope, GST fusion or His₆ and affinity purified with myc antibody, glutathione resin or Ni-resin, respectively. Such tags can be joined to the nucleotide sequence encoding a soluble PH20 as described elsewhere herein, which can facilitate purification of soluble proteins. For example, a modified PH20 polypeptide can be expressed as a recombinant protein with one or more additional polypeptide domains added to facilitate protein purification. Such purification facilitating domains include, but are not limited to, metal chelating peptides such as histidine-tryptophan modules that allow purification on immobilized metals, protein A domains that allow purification on immobilized immunoglobulin and the domain utilized in the FLAGS extension/affinity purification system (Immunex Corp., Seattle Wash.). The inclusion of a cleavable linker sequence such as Factor XA or enterokinase (Invitrogen, San Diego, Calif.) between the purification domain and the expressed PH20 polypeptide is useful to facilitate purification. One such expression vector provides for expression of a fusion protein containing a PH20 polypeptide in and an enterokinase cleavage site. The histidine residues facilitate purification on IMIAC (immobilized metal ion affinity chromatography), while the enterokinase cleavage site provides a means for purifying the polypeptide from the fusion protein.

Purity can be assessed by any method known in the art including gel electrophoresis, orthogonal HPLC methods, staining and spectrophotometric techniques. The expressed and purified protein can be analyzed using any assay or method known to one of skill in the art, for example, any described in Section G. These include assays based on the physical and/or functional properties of the protein, including, but not limited to, analysis by gel electrophoresis, immunoassay and assays of hyaluronidase activity.

Depending on the expression system and host cells used, the resulting polypeptide can be heterogeneous due to peptidases present in the culture medium upon production and purification. For example, culture of soluble PH20 in CHO cells can result in a mixture of heterogeneous polypeptides.

6. Modification of Polypeptides by PEGylation

Polyethylene glycol (PEG) has been widely used in biomaterials, biotechnology and medicine primarily because PEG is a biocompatible, nontoxic, water-soluble polymer that is typically nonimmunogenic (Zhao and Harris, ACS Symposium Series 680: 458-72, 1997). In the area of drug delivery, PEG derivatives have been widely used in covalent attachment (i.e., “PEGylation”) to proteins to reduce immunogenicity, proteolysis and kidney clearance and to enhance solubility (Zalipsky, Adv. Drug Del. Rev. 16:157-82, 1995). Similarly, PEG has been attached to low molecular weight, relatively hydrophobic drugs to enhance solubility, reduce toxicity and alter biodistribution. Typically, PEGylated drugs are injected as solutions.

A closely related application is synthesis of crosslinked degradable PEG networks or formulations for use in drug delivery since much of the same chemistry used in design of degradable, soluble drug carriers can also be used in design of degradable gels (Sawhney et al., Macromolecules 26: 581-87, 1993). It also is known that intermacromolecular complexes can be formed by mixing solutions of two complementary polymers. Such complexes are generally stabilized by electrostatic interactions (polyanion-polycation) and/or hydrogen bonds (polyacid-polybase) between the polymers involved, and/or by hydrophobic interactions between the polymers in an aqueous surrounding (Krupers et al., Eur. Polym J. 32:785-790, 1996). For example, mixing solutions of polyacrylic acid (PAAc) and polyethylene oxide (PEO) under the proper conditions results in the formation of complexes based mostly on hydrogen bonding. Dissociation of these complexes at physiologic conditions has been used for delivery of free drugs (i.e., non-PEGylated). In addition, complexes of complementary polymers have been formed from both homopolymers and copolymers.

Numerous reagents for PEGylation have been described in the art. Such reagents include, but are not limited to, reaction of the polypeptide with N-hydroxysuccinimidyl (NHS) activated PEG, succinimidyl mPEG, mPEG₂-N-hydroxysuccinimide, mPEG succinimidyl alpha-methylbutanoate, mPEG succinimidyl propionate, mPEG succinimidyl butanoate, mPEG carboxymethyl 3-hydroxybutanoic acid succinimidyl ester, homobifunctional PEG-succinimidyl propionate, homobifunctional PEG propionaldehyde, homobifunctional PEG butyraldehyde, PEG maleimide, PEG hydrazide, p-nitrophenyl-carbonate PEG, mPEG-benzotriazole carbonate, propionaldehyde PEG, mPEG butryaldehyde, branched mPEG₂ butyraldehyde, mPEG acetyl, mPEG piperidone, mPEG methylketone, mPEG “linkerless” maleimide, mPEG vinyl sulfone, mPEG thiol, mPEG orthopyridylthioester, mPEG orthopyridyl disulfide, Fmoc-PEG-NHS, Boc-PEG-NHS, vinylsulfone PEG-NHS, acrylate PEG-NHS, fluorescein PEG-NHS, and biotin PEG-NHS (see e.g., Monfardini et al., Bioconjugate Chem. 6:62-69, 1995; Veronese et al., J. Bioactive Compatible Polymers 12:197-207, 1997; U.S. Pat. No. 5,672,662; U.S. Pat. No. 5,932,462; U.S. Pat. No. 6,495,659; U.S. Pat. No. 6,737,505; U.S. Pat. No. 4,002,531; U.S. Pat. No. 4,179,337; U.S. Pat. No. 5,122,614; U.S. Pat. No. 5,324,844; U.S. Pat. No. 5,446,090; U.S. Pat. No. 5,612,460; U.S. Pat. No. 5,643,575; U.S. Pat. No. 5,766,581; U.S. 5,795, 569; U.S. Pat. No. 5,808,096; U.S. Pat. No. 5,900,461; U.S. Pat. No. 5,919,455; U.S. Pat. No. 5,985,263; U.S. Pat. No. 5,990,237; U.S. Pat. No. 6,113,906; U.S. Pat. No. 6,214,966; U.S. Pat. No. 6,258,351; U.S. Pat. No. 6,340,742; U.S. Pat. No. 6,413,507; U.S. Pat. No. 6,420,339; U.S. Pat. No. 6,437,025; U.S. Pat. No. 6,448,369; U.S. Pat. No. 6,461,802; U.S. Pat. No. 6,828,401; U.S. Pat. No. 6,858,736; U.S. 2001/0021763; U.S. 2001/0044526; U.S. 2001/0046481; U.S. 2002/0052430; U.S. 2002/0072573; U.S. 2002/0156047; U.S. 2003/0114647; U.S. 2003/0143596; U.S. 2003/0158333; U.S. 2003/0220447; U.S. 2004/0013637; U.S. 2004/0235734; WO05000360; U.S. 2005/0114037; U.S. 2005/0171328; U.S. 2005/0209416; EP 1064951; EP 0822199; WO 01076640; WO 0002017; WO 0249673; WO 9428024; and WO 0187925).

In one example, the polyethylene glycol has a molecular weight ranging from about 3 kD to about 50 kD, and typically from about 5 kD to about 30 kD. Covalent attachment of the PEG to the drug (known as “PEGylation”) can be accomplished by known chemical synthesis techniques. For example, the PEGylation of protein can be accomplished by reacting NHS-activated PEG with the protein under suitable reaction conditions.

While numerous reactions have been described for PEGylation, those that are most generally applicable confer directionality, utilize mild reaction conditions, and do not necessitate extensive downstream processing to remove toxic catalysts or bi-products. For instance, monomethoxy PEG (mPEG) has only one reactive terminal hydroxyl, and thus its use limits some of the heterogeneity of the resulting PEG-protein product mixture. Activation of the hydroxyl group at the end of the polymer opposite to the terminal methoxy group is generally necessary to accomplish efficient protein PEGylation, with the aim being to make the derivatised PEG more susceptible to nucleophilic attack. The attacking nucleophile is usually the epsilon-amino group of a lysyl residue, but other amines also can react (e.g., the N-terminal alpha-amine or the ring amines of histidine) if local conditions are favorable. A more directed attachment is possible in proteins containing a single lysine or cysteine. The latter residue can be targeted by PEG-maleimide for thiol-specific modification. Alternatively, PEG hydrazide can be reacted with a periodate oxidized hyaluronan-degrading enzyme and reduced in the presence of NaCNBH₃. More specifically, PEGylated CMP sugars can be reacted with a hyaluronan-degrading enzyme in the presence of appropriate glycosyl-transferases. One technique is the “PEGylation” technique where a number of polymeric molecules are coupled to the polypeptide in question. When using this technique, the immune system has difficulties in recognizing the epitopes on the polypeptide's surface responsible for the formation of antibodies, thereby reducing the immune response. For polypeptides introduced directly into the circulatory system of the human body to give a particular physiological effect (i.e., pharmaceuticals) the typical potential immune response is an IgG and/or IgM response, while polypeptides which are inhaled through the respiratory system (i.e., industrial polypeptide) potentially can cause an IgE response (i.e., allergic response). One of the theories explaining the reduced immune response is that the polymeric molecule(s) shield(s) epitope(s) on the surface of the polypeptide responsible for the immune response leading to antibody formation. Another theory or at least a partial factor is that the heavier the conjugate is, the more reduced the resulting immune response is.

Typically, to make the PEGylated PH20 polypeptide provided herein, PEG moieties are conjugated, via covalent attachment, to the polypeptides. Techniques for PEGylation include, but are not limited to, specialized linkers and coupling chemistries (see e.g., Roberts, Adv. Drug Deliv. Rev. 54:459-476, 2002), attachment of multiple PEG moieties to a single conjugation site (such as via use of branched PEGs; see e.g., Guiotto et al., Bioorg. Med. Chem. Lett. 12:177-180, 2002), site-specific PEGylation and/or mono-PEGylation (see e.g., Chapman et al., Nature Biotech. 17:780-783, 1999), and site-directed enzymatic PEGylation (see e.g., Sato, Adv. Drug Deliv. Rev., 54:487-504, 2002). Methods and techniques described in the art can produce proteins having 1, 2, 3, 4, 5, 6, 7, 8, 9, 10 or more than 10 PEG or PEG derivatives attached to a single protein molecule (see e.g., U.S. 2006/0104968).

As an exemplary illustrative method for making a PEGylated PH20 polypeptide, PEG aldehydes, succinimides and carbonates have each been applied to conjugate PEG moieties, typically succinimidyl PEGs, to rHuPH20. For example, rHuPH20 has been conjugated with exemplary succinimidyl methoxyPEG (mPEG) reagents including mPEG-Succinimidyl Propionates (mPEG-SPA), mPEG-Succinimidyl Butanoates (mPEG-SBA), and (for attaching “branched” PEGs) mPEG2-N-Hydroxylsuccinimide. These PEGylated succinimidyl esters contain different length carbon backbones between the PEG group and the activated cross-linker, and either a single or branched PEG group. These differences can be used, for example, to provide for different reaction kinetics and to potentially restrict sites available for PEG attachment to rHuPH20 during the conjugation process.

Succinimidyl PEGs (as above) containing either linear or branched PEGs can be conjugated to PH20. PEGs can used to generate PH20s reproducibly containing molecules having, on the average, between about three to six or three to six PEG molecules per hyaluronidase. Such PEGylated rHuPH20 compositions can be readily purified to yield compositions having specific activities of approximately 25,000 or 30,000 Unit/mg protein hyaluronidase activity, and being substantially free of non-PEGylated PH20 (less than 5% non-PEGylated).

Using various PEG reagents, exemplary versions of a PEGylated PH20 polypeptide can be prepared, for example, using mPEG-SBA (30 kD), mPEG-SMB (30 kD), and branched versions based on mPEG2-NHS (40 kD) and mPEG2-NHS (60 kD). PEGylated versions of PH20 can be generated using NHS chemistries, as well as carbonates, and aldehydes, using each of the following reagents: mPEG2-NHS-40K branched, mPEG-NHS-10K branched, mPEG-NHS-20K branched, mPEG2-NHS-60K branched; mPEG-SBA-5K, mPEG-SBA-20K, mPEG-SBA-30K; mPEG-SMB-20K, mPEG-SMB-30K; mPEG-butyrldehyde; mPEG-SPA-20K, mPEG-SPA-30K; and PEG-NHS-5K-biotin. PEGylated PH20 also can be prepared using PEG reagents available from Dowpharma, a division of Dow Chemical Corporation; including PH20 polypeptides PEGylated with Dowpharma's p-nitrophenyl-carbonate PEG (30 kDa) and with propionaldehyde PEG (30 kDa).

In one example, the PEGylation includes conjugation of mPEG-SBA, for example, mPEG-SBA-30K (having a molecular weight of about 30 kDa) or another succinimidyl ester of a PEG butanoic acid derivative, to a PH20 polypeptide. Succinimidyl esters of PEG butanoic acid derivatives, such as mPEG-SBA-30K readily couple to amino groups of proteins. For example, covalent conjugation of m-PEG-SBA-30K and rHuPH20 (which is approximately 60 KDa in size) provides stable amide bonds between rHuPH20 and mPEG, as shown in Scheme 1, below.

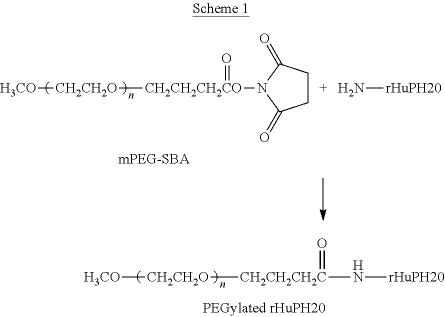

Typically, the mPEG-SBA-30K or other PEG is added to the PH20 polypeptide at a PEG:polypeptide molar ratio of 10:1 in a suitable buffer, e.g., 130 mM NaCl/10 mM HEPES at pH 6.8 or 70 mM phosphate buffer, pH 7, followed by sterilization, e.g., sterile filtration, and continued conjugation, for example, with stirring, overnight at 4° C. in a cold room. In one example, the conjugated PEG-PH20 is concentrated and buffer-exchanged.

Other methods of coupling succinimidyl esters of PEG butanoic acid derivatives, such as mPEG-SBA-30K are known in the art (see e.g., U.S. Pat. No. 5,672,662; U.S. Pat. No. 6,737,505; and U.S. 2004/0235734). For example, a polypeptide, such as a PH20 polypeptide, can be coupled to an NHS activated PEG derivative by reaction in a borate buffer (0.1 M, pH 8.0) for one hour at 4° C. The resulting PEGylated protein can be purified by ultrafiltration. Another method reacts polypeptide with mPEG-SBA in deionized water to which triethylamine is added to raise the pH to 7.2-9. The resulting mixture is stirred at room temperature for several hours to complete the PEGylation.

Methods for PEGylation of PH20 polypeptides, including, for example, animal-derived hyaluronidases and bacterial hyaluronan-degrading enzymes, are known to one of skill in the art. See, for example, European Patent No. EP 0400472, which describes the PEGylation of bovine testes hyaluronidase and chondroitin ABC lyase. Also, U.S. Publication No. 2006014968 describes PEGylation of a human hyaluronidase derived from human PH20. For example, the PEGylated hyaluronan-degrading enzyme generally contains at least 3 PEG moieties per molecule. In some examples, the PH20 polypeptide contains three to six PEG molecules. In other examples, the enzyme can have a PEG to protein molar ratio between 5:1 and 9:1, for example, 7:1.

### F. PHARMACEUTICAL COMPOSITIONS AND FORMULATIONS, DOSAGES AND ADMINISTRATION

Pharmaceutical compositions of any of the modified PH20 polypeptides set forth in Section C above are provided herein for administration. Pharmaceutical compositions, in particular liquid formulations, can be limited by the stability of the active agent, which can be susceptible to effects of storage conditions (time or length of storage, temperature and/or agitation) and/or formulation components contained in the composition. In particular, many pharmaceutical compositions require refrigeration for storage, or are stable without refrigeration for a limited time. For example, a commercial preparation of a recombinant soluble PH20 hyaluronidase (Hylenex®) is recommended for storage at room temperatures less than or equal to 25° C. for a time period not to exceed 48 hours. This can limit the applications of PH20 hyaluronidase containing pharmaceutical compositions. In particular, shipping and handling practices often require or otherwise expose a pharmaceutical composition to ambient temperatures of 18° C. to 25° C. or greater than 25° C. for more than 48 hours. Also, sustained delivery devices, such as implantable devices, also require exposure of the enzyme to elevated temperatures (e.g. 30° C. to 37° C.) for periods of time that can be destabilizing to the protein. Finally, refrigeration is not always a convenient option in many regions or countries, which can further expose the pharmaceutical composition to elevated ambient temperatures greater than 25° C. that can be destabilizing to the protein. This is particularly a concern in tropical climates.

The pharmaceutical compositions herein that contain any of the modified PH20 uber-thermophiles provided herein, are stable as a liquid formulation for prolonged periods of time greater than 48 hours under non-refrigerated conditions. Hence, the pharmaceutical compositions exhibit thermal stability (i.e. active agent retains at least 50% of the hyaluronidase activity) for at least 72 hours, 96 hours, 120 hours, 144 hours, one week, two weeks, three weeks, one month, two months, three months, four months, five months, six months or more under non-refrigerated conditions. For example, the pharmaceutical compositions exhibit the thermal stability under room temperature or elevated ambient temperature conditions, such as temperature conditions that exist in tropical climates. Such activity can be retained upon fluctuating temperature conditions that exist in non-refrigerated environments. For example, the modified PH20 uber-thermophiles provided herein are stable (i.e. active agent retains at least 50% of the hyaluronidase activity) as a liquid formulation at temperatures in the range of 18° C. to 25° C. for at least 72 hours, for at least 72 hours, 96 hours, 120 hours, 144 hours, one week, two weeks, three weeks, one month, two months, three months, four months, five months, six months or more. In other examples, the modified PH20 uber-thermophiles provided herein are stable (i.e. active agent retains at least 50% of the hyaluronidase activity) as a liquid formulation at temperatures greater than 25° C. for at least 72 hours, for at least 72 hours, 96 hours, 120 hours, 144 hours, one week, two weeks, three weeks, one month, two months, three months, four months, five months, six months or more.

In particular, the thermal stability of the pharmaceutical compositions provided herein is achieved without refrigeration in the presence of continuous, variable or intermittent temperatures greater than 25° C. In one example, the pharmaceutical compositions provided herein exhibit thermal stability under non-refrigerated conditions that expose the composition to continuous, variable or intermittent temperatures of greater than 25° C. for at least 72 hours, 96 hours, 120 hours, 144 hours, one week, two weeks, three weeks, one month, two months, three months, four months, five months, six months or more. For example, the pharmaceutical compositions provided herein exhibit thermal stability under non-refrigerated conditions that expose the composition to continuous, variable or intermittent temperatures of between 28° C. to 42° C. or 30° C. to 37° C., each exclusive, for at least 72 hours, 96 hours, 120 hours, 144 hours, one week, two weeks, three weeks, one month, two months, three months, four months, five months, six months or more.

The increased stability is characterized by improved storage time, decreased fragmentation, and/or decreased aggregate formation, while still retaining the activity of the active agent(s), e.g., the PH20 hyaluronidase. Such formulations can be provided as “ready-to use” liquid formulations without further reconstitution and/or without any requirement for further dilution. In some examples, the formulations also can be prepared in a lyophilized or concentrated form.

1. Formulations—Liquids, Injectables, Emulsions

Pharmaceutically acceptable compositions are prepared in view of approvals for a regulatory agency or other agency prepared in accordance with generally recognized pharmacopeia for use in animals and in humans. Typically, the compounds are formulated into pharmaceutical compositions using techniques and procedures well known in the art (see e.g., Ansel Introduction to Pharmaceutical Dosage Forms, Fourth Edition, 1985, 126).

The formulation generally is made to suit the route of administration. Compositions can take the form of solutions, suspensions, emulsions, tablets, pills, capsules, powders, and sustained release formulations. A composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, and other such agents. Topical formulations also are contemplated. The formulation should suit the mode of administration.

Parenteral administration, generally characterized by injection or infusion, either subcutaneously, intramuscularly, intravenously or intradermally is contemplated herein. Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. Injectables can be prepared in conventional forms, either as liquid solutions or suspensions, solid forms suitable for solution or suspension in liquid prior to injection, or as emulsions. For example, the compositions containing a modified PH20 polypeptide, formulated separately or co-formulated with another therapeutic agent, can be provided as a pharmaceutical preparation in liquid form as a solution, syrup or suspension. In liquid form, the pharmaceutical preparations can be provided as a concentrated preparation to be diluted to a therapeutically effective concentration before use. Generally, the preparations are provided in a dosage form that does not require dilution for use. In another example, pharmaceutical preparations can be presented in lyophilized form for reconstitution with water or other suitable vehicle before use.

Injectables are designed for local and systemic administration. For purposes herein, local administration is desired for direct administration to the affected interstitium. The solutions can be either aqueous or nonaqueous. If administered intravenously, suitable carriers include physiological saline or phosphate buffered saline (PBS), and solutions containing thickening and solubilizing agents, such as glucose, polyethylene glycol, and polypropylene glycol and mixtures thereof.

The concentration of the pharmaceutically active compound is adjusted so that an injection or infusion provides an effective amount to produce the desired pharmacological effect. The exact dose depends on the age, weight and condition of the patient or animal as is known in the art. The unit-dose parenteral preparations can be packaged in, for example, an ampoule, a cartridge, a vial or a syringe with a needle. The volume of liquid solution or reconstituted powder preparation, containing the pharmaceutically active compound, is a function of the disease to be treated and the particular article of manufacture chosen for package. All preparations for parenteral administration must be sterile, as is known and practiced in the art. The percentage of active compound contained in such parenteral compositions is highly dependent on the specific nature thereof, as well as the activity of the compound and the needs of the subject.

Pharmaceutical compositions can include carriers or other excipients. For example, pharmaceutical compositions provided herein can contain any one or more of a diluents(s), adjuvant(s), antiadherent(s), binder(s), coating(s), filler(s), flavor(s), color(s), lubricant(s), glidant(s), preservative(s), detergent(s), sorbent(s) or sweetener(s) and a combination thereof or vehicle with which a modified PH20 polypeptide is administered. For example, pharmaceutically acceptable carriers or excipients used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances. Formulations, including liquid preparations, can be prepared by conventional means with pharmaceutically acceptable additives or excipients.

Examples of suitable pharmaceutical carriers are described in “Remington's Pharmaceutical Sciences” by E. W. Martin. Such compositions will contain a therapeutically effective amount of the compound, generally in purified form, together with a suitable amount of carrier so as to provide the form for proper administration to the patient. Such pharmaceutical carriers can be sterile liquids, such as water or oils, including those of petroleum, animal, vegetable or synthetic origin, such as peanut oil, soybean oil, mineral oil, and sesame oil. Water is a typical carrier when the pharmaceutical composition is administered intravenously. Saline solutions and aqueous dextrose and glycerol solutions also can be employed as liquid carriers, particularly for injectable solutions. Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Suspending and dispersing agents include, but are not limited to, sorbitol syrup, cellulose derivatives or hydrogenated edible fats, sodium carboxymethylcellulose, hydroxypropyl methylcellulose and polyvinylpyrrolidone. Emulsifying agents include, but are not limited to, lecithin or acacia. Detergents include, but are not limited to, Polysorbate 80 (TWEEN 80). Non-aqueous vehicles include, but are not limited to, almond oil, oily esters, or fractionated vegetable oils. Anti-microbial agents or preservatives include, but are not limited to, methyl or propyl-p-hydroxybenzoates or sorbic acid, m-cresol, phenol. A diluent includes, but is not limited to, lactose, sucrose, dicalcium phosphate, or carboxymethylcellulose. A lubricant includes, but is not limited to, magnesium stearate, calcium stearate or talc. A binder includes, but is not limited to, starch, natural gums, such as gum acacia, gelatin, glucose, molasses, polyvinylpyrrolidine, celluloses and derivatives thereof, povidone, crospovidones and other such binders known to those of skill in the art. Isotonic agents include, but are not limited to, sodium chloride and dextrose. Buffers include, but are not limited to, phosphate and citrate. Antioxidants include sodium bisulfate. Local anesthetics include procaine hydrochloride. A sequestering or chelating agent of metal ions includes EDTA. Other suitable pharmaceutical excipients include, but are not limited to, starch, glucose, lactose, dextrose, sucrose, gelatin, malt, rice, flour, chalk, silica gel, sodium stearate, glycerol monostearate, talc, sodium chloride, dried skim milk, glycerol, propylene, glycol, saline, water, and ethanol. Pharmaceutical carriers also include ethyl alcohol, polyethylene glycol and propylene glycol for water miscible vehicles and sodium hydroxide, hydrochloric acid, citric acid or lactic acid for pH adjustment. A composition, if desired, also can contain minor amounts of non-toxic auxiliary substances such as wetting or emulsifying agents, or pH buffering agents, for example, acetate, sodium citrate, cyclodextrin derivatives, sorbitan monolaurate, triethanolamine sodium acetate, triethanolamine oleate, stabilizers, solubility enhancers, and other such agents such as for example, sodium acetate, sodium phosphate, sorbitan monolaurate, triethanolamine oleate and cyclodextrins.

In particular, antimicrobial agents (e.g., preservatives) in bacteriostatic or fungistatic concentrations (e.g., an anti-microbial effective amount) can be added to parenteral preparations packaged in multiple-dose containers, which include phenols or cresols, mercurials, benzyl alcohol, chlorobutanol, methyl and propyl p-hydroxybenzoic acid esters, thimerosal, benzalkonium chloride and benzethonium chloride.

The volume of the formulations, including the separately formulated or co-formulated PH20-containing formulations provided herein, can be any volume suitable for the container in which it is provided. In some examples, the formulations are provided in a vial, syringe, or any other suitable container. For example, the formulations provided herein are between or about between 0.1 mL to 500 mL, such as 0.1 mL to 100 mL, 1 mL to 100 mL, 0.1 mL to 50 mL, such as at least or about at least or about or is 0.1 mL, 1 mL, 2 mL, 3 mL, 4 mL, 5 mL, 10 mL, 15 mL, 20 mL, 30 mL, 40 mL, 50 mL or more.

a. Lyophilized Powders

Of interest herein are lyophilized powders, which can be reconstituted for administration as solutions, emulsions and other mixtures. They may also be reconstituted and formulated as solids or gels.

The sterile, lyophilized powder is prepared by dissolving a compound of enzyme in a buffer solution. The buffer solution may contain an excipient which improves the stability or other pharmacological component of the powder or reconstituted solution, prepared from the powder. Subsequent sterile filtration of the solution followed by lyophilization under standard conditions known to those of skill in the art provides the desired formulation. A liquid formulation as described herein above can be prepared. The resulting mixture is sterile filtered or treated to remove particulates and to insure sterility, and apportioned into vials for lyophilization. For example, the lyophilized powder can be prepared by dissolving an excipient, such as dextrose, sorbitol, fructose, corn syrup, xylitol, glycerin, glucose, sucrose or other suitable agent, in a suitable buffer, such as citrate, sodium or potassium phosphate or other such buffer known to those of skill in the art. Then, a selected enzyme is added to the resulting mixture, and stirred until it dissolves.

Each vial is made to contain a single dosage or multiple dosages of the compound. The lyophilized powder can be stored under appropriate conditions, such as at about 4° C. to room temperature. Reconstitution of this lyophilized powder with an appropriate buffer solution provides a formulation for use in parenteral administration.

b. Exemplary Formulations

Single dose formulations of PH20 are known in the art. For example, Hylenex® recombinant (hyaluronidase human injection) contains, per mL, 8.5 mg NaCl (145 mM), 1.4 mg dibasic sodium phosphate (9.9 mM), 1.0 mg human albumin, 0.9 mg edetate disodium (2.4 mM), 0.3 mg CaCl₂ (2.7 mM) and NaOH to adjust the pH to 7.4. Other formulations of human soluble hyaluronidase, such as the rHuPH20 formulations described in U.S. Pat. Pub. No. US2011/0053247, include 130 mM NaCl, 10 mM HEPES, pH 7.0; or 10 mM histidine, 130 mM NaCl, pH 6.0. Any of the modified PH20 polypeptides provided herein can be similarly formulated.

In addition to a therapeutically effective amount of a modified PH20 polypeptide and/or other therapeutic agent, exemplary pharmaceutical compositions provided herein, including separately formulated- and co-formulated-PH20 containing formulations are prepared at a requisite pH to maintain the stability of the active agent(s) (e.g., PH20 hyaluronidase and/or other co-formulated therapeutic agent). Such formulations also can contain a concentration of salt, such as NaCl.

For multi-dose formulations and other formulations stored for a prolonged time, the compositions generally also contain one or more preservatives. Generally, because the PH20 hyaluronidases are thermally stable, further stabilizing agents are not required. Depending on the application and purpose of the composition, however, further stabilizing agents and other excipients also can be included. Such inclusion is within the level of a skilled artisan to empirically determine Exemplary components are described below.

i. pH and Buffer

In examples herein, the pharmaceutical compositions provided herein are prepared at a pH of between or about between 6.5 to 7.8 such as between or about between 6.5 to 7.2, 7.0 to 7.8, 7.0 to 7.6 or 7.2 to 7.4. Reference to pH herein is based on measurement of pH at room temperature. It is understood that the pH can change during storage over time, but typically will remain between or between about pH 6.5 to or to about 7.8. For example, the pH can vary by ±0.1, 0.2, 0.3, 0.4, 0.5, 0.6, 0.7, 0.8, 0.9, 1.0, 1.2, 1.3, 1.4, 1.5 or more. Exemplary co-formulations provided herein have a pH of or of about 7.0±0.2, 7.1±0.2, 7.2±0.2, 7.3±0.2, 7.4±0.2, 7.5±0.2 or 7.6±0.2 when prepared. If necessary, pH can be adjusted using acidifying agents to lower the pH or alkalizing agents to increase the pH. Exemplary acidifying agents include, but are not limited to, acetic acid, citric acid, sulfuric acid, hydrochloric acid, monobasic sodium phosphate solution, and phosphoric acid. Exemplary alkalizing agents include, but are not limited to, dibasic sodium phosphate solution, sodium carbonate, or sodium hydroxide.

The compositions are generally prepared using a buffering agent that maintains the pH range. Any buffer can be used in formulations provided herein so long as it does not adversely affect the stability of the active agent(s) (e.g., PH20 hyaluronidase), and supports the requisite pH range required. Examples of particularly suitable buffers include Tris, succinate, acetate, phosphate buffers, citrate, aconitate, malate and carbonate. Those of skill in the art, however, will recognize that formulations provided herein are not limited to a particular buffer, so long as the buffer provides an acceptable degree of pH stability, or “buffer capacity” in the range indicated. Generally, a buffer has an adequate buffer capacity within about 1 pH unit of its pK (Lachman et al. In: The Theory and Practice of Industrial Pharmacy 3rd Edn. (Lachman, L., Lieberman, H A. and Kanig, J. L., Eds.), Lea and Febiger, Philadelphia, p. 458-460, 1986). Buffer suitability can be estimated based on published pK tabulations or can be determined empirically by methods well known in the art. The pH of the solution can be adjusted to the desired endpoint within the range as described above, for example, using any acceptable acid or base.

Buffers that can be included in the co-formulations provided herein include, but are not limited to, Tris (Tromethamine), histidine, phosphate buffers, such as dibasic sodium phosphate, and citrate buffers. Such buffering agents can be present in the compositions at concentrations between or about between 1 mM to 100 mM, such as 10 mM to 50 mM or 20 mM to 40 mM, such as at or about 30 mM. For example, such buffering agents can be present in the compositions in a concentration of or about 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 25 mM, 30 mM, 35 mM, 40 mM, 45 mM, 50 mM, 55 mM, 60 mM, 65 mM, 70 mM, 75 mM, or more.

ii. Salt (e.g. NaCl)

In examples herein, the pharmaceutical compositions provided herein contain a concentration of salt, such as sodium chloride (NaCl), which can be required for activity of the PH20 hyaluronidase). In particular examples herein, the pharmaceutical compositions, including the separately formulated or co-formulated PH20-containing formulations provided herein, contain salt, such as NaCl, at a concentration of between or about between 10 mM to 200 mM, 50 mM to 200 mM, 80 mM to 160 mM, 100 mM to 140 mM, 120 mM to 180 mM, 140 mM to 180 mM, 120 mM to 160 mM, 130 mM to 150 mM, 80 mM to 140 mM.

Low salt concentrations of generally less than 120 mM can have deleterious effects on PH20 activity over time and depending on temperature conditions. Hence, the absence of salt (e.g. NaCl) or a low concentration of salt (e.g. NaCl) can result in instability of the protein. In the pharmaceutical compositions provided herein, due to the thermal stability achieved by the modified PH20 polypeptides provided herein, lower concentrations of salt (e.g. NaCl) can be suitable to maintain and preserve hyaluronidase activity. Hence, in examples herein, pharmaceutical compositions can contain an amount of salt (e.g. NaCl) that is less than 130 mM, such as such as 10 mM to 120 mM, 50 mM to 120 mM, 80 mM to 120 mM, 50 mM to 100 mM, 50 mM to 90 mM, 80 mM to 100 mM or 10 mM to 50 mM, each inclusive.

iii. Preservative(s)

In examples herein, multi-dose formulations or formulations stored for prolonged periods contain an anti-microbially effective amount of preservative or mixture of preservatives in an amount to have a bacteriostatic or fungistatic effect. The amount of preservative(s) is an amount that maintains the activity of the active agent(s) (e.g. PH20 hyaluronidase).

Non-limiting examples of preservatives that can be included in the compositions or co-formulations provided herein include, but are not limited to, phenol, meta-cresol (m-cresol), methylparaben, benzyl alcohol, thimerosal, benzalkonium chloride, 4-chloro-1-butanol, chlorhexidine dihydrochloride, chlorhexidine digluconate, L-phenylalanine, EDTA, bronopol (2-bromo-2-nitropropane-1,3-diol), phenylmercuric acetate, glycerol (glycerin), imidurea, chlorhexidine, sodium dehydroacetate, ortho-cresol (o-cresol), para-cresol (p-cresol), chlorocresol, cetrimide, benzethonium chloride, ethylparaben, propylparaben or butylparaben and any combination thereof. For example, formulations provided herein can contain a single preservative. In other examples, the formulations contain at least two different preservatives or at least three different preservatives. For example, formulations provided herein can contain two preservatives such as L-phenylalanine and m-cresol, L-phenylalanine and methylparaben, L-phenylalanine and phenol, m-cresol and methylparaben, phenol and methylparaben, m-cresol and phenol or other similar combinations.

In the formulations provided herein, the total amount of the one or more preservative agents as a percentage (%) of mass concentration (w/v) in the formulation can be, for example, between from or between about from 0.1% to 0.4%, such as 0.1% to 0.3%, 0.15% to 0.325%, 0.15% to 0.25%, 0.1% to 0.2%, 0.2% to 0.3%, or 0.3% to 0.4%. Generally, the formulations contain less than 0.4% (w/v) preservative. For example, the co-formulations provided herein contain at least or about at least 0.1%, 0.12%, 0.125%, 0.13%, 0.14%, 0.15%, 0.16% 0.17%, 0.175%, 0.18%, 0.19%, 0.2%, 0.25%, 0.3%, 0.325%, 0.35% but less than 0.4% total preservative.

iv. Stabilizers

In examples herein, the pharmaceutical compositions provided herein optionally can contain one or more other stabilizing agent to maintain the stability of the PH20 hyaluronidase. In some examples provided herein, pharmaceutical compositions do not contain a stabilizing agent that is an amino acids, amino acid derivatives, amines, sugars, polyols, surfactants, a hyaluronidase inhibitor or other substrate or an albumin protein (e.g. human albumin) In other examples provided herein, the pharmaceutical compositions contain one or more stabilizing agents from among a stabilizing agent that is an amino acids, amino acid derivatives, amines, sugars, polyols, surfactants, a hyaluronidase inhibitor or other substrate or an albumin protein (e.g. human albumin)

Included among the types of stabilizers that can optionally be contained in the formulations provided herein are amino acids, amino acid derivatives, amines, sugars, polyols, salts and buffers, surfactants, and other agents. For example, the formulations herein contain at least contain a surfactant and an appropriate buffer. Optionally, the formulations provided herein can contain other additional stabilizers. Other components include, for example, one or more tonicity modifiers, one or more anti-oxidation agents, or other stabilizer.

Exemplary amino acid stabilizers, amino acid derivatives or amines include, but are not limited to, L-Arginine, Glutamine, Glycine, Lysine, Methionine, Proline, Lys-Lys, Gly-Gly, Trimethylamine oxide (TMAO) or betaine. Exemplary sugars and polyols include, but are not limited to, glycerol, sorbitol, mannitol, inositol, sucrose or trehalose. Exemplary salts and buffers include, but are not limited to, magnesium chloride, sodium sulfate, Tris such as Tris (100 mM), or sodium Benzoate. Exemplary surfactants include, but are not limited to, poloxamer 188 (e.g., Pluronic® F68), polysorbate 80 (PS80), polysorbate 20 (PS20). Other stabilizers include, but are not limited to, hyaluronic acid (HA), human serum albumin (HSA), phenyl butyric acid, taurocholic acid, polyvinylpyrolidone (PVP) or zinc.

For example, surfactants can inhibit aggregation of PH20 and minimize absorptive loss. The surfactants generally are non-ionic surfactants. Surfactants that can be included in the formulations herein include, but are not limited to, partial and fatty acid esters and ethers of polyhydric alcohols such as of glycerol, or sorbitol, poloxamers and polysorbates. For example, exemplary surfactants in the -formulations herein include any one or more of poloxamer 188 (PLURONICS® such as PLURONIC® F68), TETRONICS®, polysorbate 20, polysorbate 80, PEG 400, PEG 3000, Tween® (e.g., Tween® 20 or Tween® 80), Triton® X-100, SPAN®, MYRJ®, BRIJ®, CREMOPHOR®, polypropylene glycols or polyethylene glycols. In some examples, the formulations herein contain poloxamer 188, polysorbate 20, polysorbate 80, generally poloxamer 188 (Pluronic® F68).

In the formulations provided herein, the total amount of the one or more surfactants as a percentage (%) of mass concentration (w/v) in the formulation can be, for example, between from or between about from 0.005% to 1.0%, such as between from or between about from 0.01% to 0.5%, such as 0.01% to 0.1% or 0.01% to 0.02%. Generally, the formulations contain at least 0.01% surfactant and contain less than 1.0%, such as less than 0.5% or less than 0.1% surfactant. For example, the formulations provided herein can contain at or about 0.001%, 0.005%, 0.01%, 0.015%, 0.02%, 0.025%, 0.03%, 0.035%, 0.04%, 0.045%, 0.05%, 0.055%, 0.06%, 0.065%, 0.07%, 0.08%, or 0.09% surfactant. In particular examples, the formulations provided herein contain or contain about 0.01% to or to about 0.05% surfactant.

Tonicity modifiers can be included in the formulation provided herein to produce a solution with the desired osmolality. The formulations provided herein have an osmolality of between or about between 245 mOsm/kg to 305 mOsm/kg. For example, the osmolality is or is about 245 mOsm/kg, 250 mOsm/kg, 255 mOsm/kg, 260 mOsm/kg, 265 mOsm/kg, 270 mOsm/kg, 275 mOsm/kg, 280 mOsm/kg, 285 mOsm/kg, 290 mOsm/kg, 295 mOsm/kg, 300 mOsm/kg or 305 mOsm/kg. In some examples, the formulations have an osmolality of or of about 275 mOsm/kg. Tonicity modifiers include, but are not limited to, glycerin, NaCl, amino acids, polyalcohols, trehalose, and other salts and/or sugars. The particular amount can be empirically determined in order to retain enzyme activity, and/or tonicity.

In other instances, glycerin (glycerol) is included in the formulations. For example, formulations provided herein typically contain less than 60 mM glycerin, such as less than 55 mM, less than 50 mM, less than 45 mM, less than 40 mM, less than 35 mM, less than 30 mM, less than 25 mM, less than 20 mM, less than 15 mM, 10 mM or less. The amount of glycerin typically depends on the amount of NaCl present: the more NaCl present in the formulation, the less glycerin is required to achieve the desired osmolality or osmolarity. Thus, for example, in formulations containing higher NaCl concentrations, little or no glycerin need be included in the formulation. In contrast, in formulations containing slightly lower NaCl concentrations, glycerin can be included. For example, formulations provided herein can contain glycerin at a concentration of 40 mM to 60 mM, such as less than 50 mM, such as 20 mM to 50 mM, for example at or about 50 mM.

The formulations provided herein also can contain antioxidants to reduce or prevent oxidation, in particular oxidation of the PH20 polypeptide. For example, oxidation can be effected by high concentrations of surfactant or hyaluronan oligomers. Exemplary antioxidants include, but are not limited to, cysteine, tryptophan and methionine. In particular examples, the anti-oxidant is methionine. The formulations provided herein can include an antioxidant at a concentration from between or from about between 5 mM to or to about 50 mM, such as 5 mM to 40 mM, 5 mM to 20 mM or 10 mM to 20 mM. For example, methionine can be provided in the formulations herein at a concentration from between or from about between 5 mM to or to about 50 mM, such as 5 mM to 40 mM, 5 mM to 20 mM or 10 mM to 20 mM. For example, an antioxidant, for example methionine, can be included at a concentration that is or is about 5 mM, 10 mM, 11 mM, 12 mM, 13 mM, 14 mM, 15 mM, 16 mM, 17 mM, 18 mM, 19 mM, 20 mM, 21 mM, 22 mM, 23 mM, 24 mM, 25 mM, 26 mM, 27 mM, 28 mM, 29 mM, 30 mM, 35 mM, 40 mM, 45 mM or 50 mM. In some examples, the formulations contain 10 mM to 20 mM methionine, such as or about 10 mM or 20 mM methionine.

The formulations provided herein also can contain an amino acid stabilizer, which contributes to the stability of the preparation. The stabilizer can be a non-polar or basic amino acid. Exemplary non-polar and basic amino acids include, but are not limited to, alanine, histidine, arginine, lysine, ornithine, isoleucine, valine, methionine, glycine and proline. For example, the amino acid stabilizer is glycine or proline, typically glycine. The stabilizer can be a single amino acid or it can be a combination of 2 or more such amino acids. The amino acid stabilizers can be natural amino acids, amino acid analogues, modified amino acids or amino acid equivalents. Generally, the amino acid is an L-amino acid. For example, when proline is used as the stabilizer, it is generally L-proline. It is also possible to use amino acid equivalents, for example, proline analogues. The concentration of amino acid stabilizer, for example glycine, included in the formulation ranges from 0.1 M to 1 M amino acid, typically 0.1 M to 0.75 M, generally 0.2 M to 0.5 M, for example, at least at or about 0.1 M, 0.15 M, 0.2 M, 0.25 M, 0.3 M, 0.35 M, 0.4 M, 0.45 M, 0.5 M, 0.6 M, 0.7 M, 0.75 M or more amino acid. The amino acid, for example glycine, can be used in a form of a pharmaceutically acceptable salt, such as hydrochloride, hydrobromide, sulfate, acetate, etc. The purity of the amino acid, for example glycine, should be at least 98%, at least 99%, or at least 99.5% or more.

In examples herein, if necessary, hyaluronidase inhibitors are included in a formulation to stabilize PH20, in particular to reduce the effects of otherwise destabilizing agents and conditions, such as, for example, low salt, high pH, the presence of preservatives and elevated temperatures, present in the formulation. Such a component generally is not required for pharmaceutical compositions containing a modified PH20 polypeptide as provided herein that exhibits increased stability under such conditions. When provided, the hyaluronidase inhibitor is provided at least at its equilibrium concentration. One of skill in the art is familiar with various classes of hyaluronidase inhibitors (see e.g., Girish et al. (2009) Current Medicinal Chemistry, 16:2261-2288, and references cited therein). One of skill in the art knows or can determine by standard methods in the art the equilibrium concentration of a hyaluronidase inhibitor in a reaction or stable composition herein.

An exemplary hyaluronidase inhibitor for use in the compositions herein is hyaluronan (HA). Hyaluronic acid (HA, also known as hyaluronan and hyaluronate) is the natural substrate for PH20. HA is a non-sulfated glycosaminoglycan that is widely distributed throughout connective, epithelial, and neural tissues. It is a polymer of up to 25,000 disaccharide units, themselves composed of D-glucuronic acid and D-N-acetylglucosamine. The molecular weight of HA ranges from about 5 kDa to 200,000 kDa. Any size HA can be used in the compositions as a stabilizer. In some examples, the HA is a disaccharide, composed of D-glucuronic acid and D-N-acetylglucosamine. In other examples, the HA is an oligosaccharide, such as a tetrasaccharide, containing 2 repeating disaccharide units, or alternatively, the HA can contain multiple repeating disaccharide units, such as 3, 4, 5, 6, 7, 8, 9, 10, 11, 12, 13, 14, 15, 16, 17, 18, 19, 20, 25, 30 or more disaccharide units. In another example, the HA used in the formulations provided herein has a molecular weight that is from or from about 5 kDa to or to about 5,000 kDa; from or from about 5 kDa to or to about 1,000 kDa; from or from about 5 kDa to or to about 500 kDa; or from or from about 5 kDa to or to about 200 kDa. Exemplary HA oligosaccharides for use in the formulations herein have a molecular weight of or of about 6.4 kDa, 74.0 kDa. or 234.4 kDa. The formulations can contain 1 mg/mL to 20 mg/mL HA, 8 mg/mL to 12 mg/mL, such as at least or about 1 mg/mL, 2 mg/mL, 3 mg/mL, 4 mg/mL, 5 mg/mL, 6 mg/mL, 7 mg/mL, 8 mg/mL, 9 mg/mL, 10 mg/mL, 11 mg/mL, 12 mg/mL, 13 mg/mL, 14 mg/mL, 15 mg/mL, 16 mg/mL, 17 mg/mL, 18 mg/mL, 19 mg/mL or 20 mg/mL or more HA. In some examples, the molar ratio of HA to PH20 is or is about 100,000:1, 95,000:1, 90,000:1, 85,000:1, 80,000:1, 75,000:1, 70,000:1, 65,000:1, 60,000:1, 55,000:1, 50,000:1, 45,000:1, 40,000:1, 35,000:1, 30,000:1, 25,000:1, 20,000:1, 15,000:1, 10,000:1, 5,000:1, 1,000:1, 900:1, 800:1, 700:1, 600:1, 500:1, 400:1, 300:1, 200:1, or 100:1 or less.

In some examples, a nicotinic compound is used as a stabilizing agent. Nicotinic compounds include, but are not limited to, nicotinamide, nicotinic acid, niacin, niacinamide, vitamin B3 and/or salts thereof and/or any combination thereof. In particular applications, the stabilizing agent can include a nicotinic compound an amino acid or amino acids (see e.g., International Publication No. WO2010149772). For example, the amino acid can be arginine, glutamic acid and/or salts thereof or combinations thereof

2. Compositions for Additional Routes of Administration

Depending upon the condition treated other routes of administration, such as topical application, transdermal patches, oral and rectal administration are also contemplated herein.

For example, pharmaceutical dosage forms for rectal administration are rectal suppositories, capsules and tablets for systemic effect. Rectal suppositories include solid bodies for insertion into the rectum which melt or soften at body temperature releasing one or more pharmacologically or therapeutically active ingredients. Pharmaceutically acceptable substances utilized in rectal suppositories are bases or vehicles and agents to raise the melting point. Examples of bases include cocoa butter (theobroma oil), glycerin-gelatin, carbowax (polyoxyethylene glycol) and appropriate mixtures of mono-, di- and triglycerides of fatty acids. Combinations of the various bases may be used. Agents to raise the melting point of suppositories include spermaceti and wax. Rectal suppositories may be prepared either by the compressed method or by molding. The typical weight of a rectal suppository is about 2 to 3 gm. Tablets and capsules for rectal administration are manufactured using the same pharmaceutically acceptable substance and by the same methods as for formulations for oral administration. Formulations suitable for rectal administration can be provided as unit dose suppositories. These can be prepared by admixing the active compound with one or more conventional solid carriers, for example, cocoa butter, and then shaping the resulting mixture.

For oral administration, pharmaceutical compositions can take the form of, for example, tablets or capsules prepared by conventional means with pharmaceutically acceptable excipients such as binding agents (e.g., pregelatinized maize starch, polyvinyl pyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulfate). The tablets can be coated by methods well-known in the art.

Formulations suitable for buccal (sublingual) administration include, for example, lozenges containing the active compound in a flavored base, usually sucrose and acacia or tragacanth; and pastilles containing the compound in an inert base such as gelatin and glycerin or sucrose and acacia.

Topical mixtures are prepared as described for the local and systemic administration. The resulting mixtures can be solutions, suspensions, emulsion or the like and are formulated as creams, gels, ointments, emulsions, solutions, elixirs, lotions, suspensions, tinctures, pastes, foams, aerosols, irrigations, sprays, suppositories, bandages, dermal patches or any other formulations suitable for topical administration.

The compounds or pharmaceutically acceptable derivatives thereof may be formulated as aerosols for topical application, such as by inhalation (see, e.g., U.S. Pat. Nos. 4,044,126, 4,414,209, and 4,364,923, which describe aerosols for delivery of a steroid useful for treatment of inflammatory diseases, particularly asthma). These formulations, for administration to the respiratory tract, can be in the form of an aerosol or solution for a nebulizer, or as a microfine powder for insufflation, alone or in combination with an inert carrier such as lactose. In such a case, the particles of the formulation will typically have diameters of less than 50 microns, or less than 10 microns.

The compounds can be formulated for local or topical application, such as for topical application to the skin and mucous membranes, such as in the eye, in the form of gels, creams, and lotions and for application to the eye or for intracisternal or intraspinal application. Topical administration is contemplated for transdermal delivery and also for administration to the eyes or mucosa, or for inhalation therapies. Nasal solutions of the active compound alone or in combination with other pharmaceutically acceptable excipients also can be administered.

Formulations suitable for transdermal administration are provided. They can be provided in any suitable format, such as discrete patches adapted to remain in intimate contact with the epidermis of the recipient for a prolonged period of time. Such patches contain the active compound in an optionally buffered aqueous solution of, for example, 0.1 to 0.2 M concentration with respect to the active compound. Formulations suitable for transdermal administration also can be delivered by iontophoresis (see, e.g., Tyle, P, Pharmaceutical Research 3(6):318-326 (1986)) and typically take the form of an optionally buffered aqueous solution of the active compound.

Pharmaceutical compositions also can be administered by controlled release formulations and/or delivery devices (see e.g., in U.S. Pat. Nos. 3,536,809; 3,598,123; 3,630,200; 3,845,770; 3,916,899; 4,008,719; 4,769,027; 5,059,595; 5,073,543; 5,120,548; 5,591,767; 5,639,476; 5,674,533 and 5,733,566).

3. Dosages and Administration

The modified PH20 polypeptides provided herein can be formulated as pharmaceutical compositions for single dosage or multiple dosage administration. The PH20 polypeptide is included in an amount sufficient to exert a therapeutically useful effect in the absence of undesirable side effects on the patient treated. The therapeutically effective concentration can be determined empirically by testing the polypeptides in known in vitro and in vivo systems such as by using the assays provided herein or known in the art (see e.g., Taliani et al. (1996) Anal. Biochem., 240: 60-67; Filocamo et al. (1997) J Virology, 71: 1417-1427; Sudo et al. (1996) Antiviral Res. 32: 9-18; Bouffard et al. (1995) Virology, 209:52-59; Bianchi et al. (1996) Anal. Biochem., 237: 239-244; Hamatake et al. (1996) Intervirology 39:249-258; Steinkuhler et al. (1998) Biochem., 37:8899-8905; D'Souza et al. (1995) J Gen. Virol., 76:1729-1736; Takeshita et al. (1997) Anal. Biochem., 247:242-246; see also e.g., Shimizu et al. (1994) J. Virol. 68:8406-8408; Mizutani et al. (1996) J. Virol. 70:7219-7223; Mizutani et al. (1996) Biochem. Biophys. Res. Commun., 227:822-826; Lu et al. (1996) Proc. Natl. Acad. Sci (USA), 93:1412-1417; Hahm et al., (1996) Virology, 226:318-326; Ito et al. (1996) J. Gen. Virol., 77:1043-1054; Mizutani et al. (1995) Biochem. Biophys. Res. Commun., 212:906-911; Cho et al. (1997) J. Virol. Meth. 65:201-207 and then extrapolated therefrom for dosages for humans.

The amount of a modified PH20 to be administered for the treatment of a disease or condition can be determined by standard clinical techniques. In addition, in vitro assays and animal models can be employed to help identify optimal dosage ranges. The precise dosage, which can be determined empirically, can depend on the particular enzyme, the route of administration, the type of disease to be treated and the seriousness of the disease.

Hence, it is understood that the precise dosage and duration of treatment is a function of the disease being treated and can be determined empirically using known testing protocols or by extrapolation from in vivo or in vitro test data. It is to be noted that concentrations and dosage values also can vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or use of compositions and combinations containing them. The compositions can be administered hourly, daily, weekly, monthly, yearly or once. Generally, dosage regimens are chosen to limit toxicity. It should be noted that the attending physician would know how to and when to terminate, interrupt or adjust therapy to lower dosage due to toxicity, or bone marrow, liver or kidney or other tissue dysfunctions. Conversely, the attending physician would also know how to and when to adjust treatment to higher levels if the clinical response is not adequate (precluding toxic side effects).

Typically, a therapeutically effective dose of a modified PH20 enzyme is at or about 10 Unit (U) to 500,000 Units, 100 Units to 100,000 Units, 500 Units to 50,000 Units, 1000 Units to 10,000 Units, 5000 Units to 7500 Units, 5000 Units to 50,000 Units, or 1,000 Units to 10,000 Units, generally 1,000 to 50,000 Units, in a stabilized solution or suspension or a lyophilized form. For example, a PH20 polypeptide, can be administered at a dose of at least or about at least or 10 U, 20 U, 30 U, 40 U, 50 U, 100 U, 150 U, 200 U, 250 U, 300 U, 350 U, 400 U, 450 U, 500 U, 600 U, 700 U, 800 U, 900 U, 1000 U, 2,000 U, 3,000 U, 4,000 U, 5,000 U or more. The formulations can be provided in unit-dose forms such as, but not limited to, ampoules, syringes and individually packaged tablets or capsules.

The PH20 enzyme can be administered alone, or with other pharmacologically effective agent(s) or therapeutic agent(s), in a total volume of 0.1-100 mL, 1-50 mL, 10-50 mL, 10-30 mL, 1-20 mL, or 1-10 mL, typically 10-50 mL. Typically, volumes of injections or infusions of a PH20-containing composition are at least or at least about 0.01 mL, 0.05 mL, 0.1 mL, 0.2 mL, 0.3 mL, 0.4 mL, 0.5 mL, 1 mL, 2 mL, 3 mL, 4 mL, 5 mL, 6 mL, 7 mL, 8 mL, 9 mL, 10 mL, 20 mL, 30 mL, 40 mL, 50 mL or more. The formulations provided herein contain a modified PH20 polypeptide in an amount between or about between 30 Units (U)/mL to 3000 U/mL, 300 U/mL to 2000 U/mL or 600 U/mL to 2000 U/mL or 600 U/mL to 1000 U/mL, such as at least or about at least 30 U/mL, 35 U/mL, 40 U/mL, 50 U/mL, 100 U/mL, 200 U/mL, 300 U/mL, 400 U/mL, 500 U/mL, 600 U/mL, 700 U/mL, 800 U/mL, 900 U/mL, 1000 U/mL, 2000 U/mL or 3000 U/mL. For example, the formulations provided herein contain a PH20 that is in an amount that is at least 100 U/mL to 1000 U/mL, for example at least or about at least or about or is 600 U/mL.

The PH20 polypeptide can be provided as a solution in an amount that is at least or about or is 100 U/mL, 150 U/mL, 200 U/mL, 300 U/mL, 400 U/mL, 500 U/mL, 600 U/mL, 800 U/mL or 1000 U/mL, or can be provided in a more concentrated form, for example in an amount that is at least or about or is 2000 U/mL, 3000 U/mL, 4000 U/mL, 5000 U/mL, 8000 U/mL, 10,000 U/mL or 20,000 U/mL for use directly or for dilution to the effective concentration prior to use. The PH20 polypeptide compositions can be provided as a liquid or lyophilized formulation.

When the PH20 is co-formulated with a therapeutic agent, dosages can be provided as a ratio of the amount of a PH20 polypeptide to the amount of therapeutic agent administered. For example, a PH20 polypeptide can be administered at 1 hyaluronidase U/therapeutic agent U (1:1) to 50:1 or more, for example, at or about 1:1, 2:1, 3:1, 4:1, 5:1, 6:1, 7:1, 8:1, 9:1, 10:1, 11:1, 12:1, 13:1, 14:1, 15:1, 20:1, 25:1, 30:1, 35:1, 40:1, 45:1, 50:1 or more.

The formulations provided herein, including co-formulations and/or stable formulations, can be prepared for single dose administration, multiple dose administration or continuous infusion administrations. Implantation of a slow-release or sustained-release system, such that a constant level of dosage is maintained (see e.g., U.S. Pat. No. 3,710,795), is also contemplated herein.

For example, formulations of pharmaceutically and therapeutically active compounds and derivatives thereof are provided for administration to humans and animals in unit dosage forms or multiple dosage forms. For example, compounds can be formulated as tablets, capsules, pills, powders, granules, sterile parenteral solutions or suspensions, oral solutions or suspensions, or oil-water emulsions containing suitable quantities of the compounds or pharmaceutically acceptable derivatives thereof. Each unit dose contains a predetermined quantity of therapeutically active compound(s) sufficient to produce the desired therapeutic effect, in association with the required pharmaceutical carrier, vehicle or diluent. Examples of unit dose forms include ampoules and syringes and individually packaged tablets or capsules. Unit dose forms can be administered in fractions or multiples thereof. A multiple dose form is a plurality of identical unit dosage forms packaged in a single container to be administered in segregated unit dose forms. Examples of multiple dose forms include vials, bottles of tablets or capsules or bottles of pints or gallons. Hence, multiple dose form is a multiple of unit doses that are not segregated in packaging. Generally, dosage forms or compositions containing active ingredient in the range of 0.005% to 100% with the balance made up from non-toxic carrier can be prepared.

Compositions provided herein typically are formulated for administration by subcutaneous route, although other routes of administration are contemplated, such as any route known to those of skill in the art including intramuscular, intraperitoneal, intravenous, intradermal, intralesional, intraperitoneal injection, epidural, vaginal, rectal, local, otic, transdermal administration or any route of administration. Formulations suited for such routes are known to one of skill in the art. Administration can be local, topical or systemic depending upon the locus of treatment. Local administration to an area in need of treatment can be achieved by, for example, but not limited to, local infusion during surgery, topical application, e.g., in conjunction with a wound dressing after surgery, by injection, by means of a catheter, by means of a suppository, or by means of an implant. Compositions also can be administered with other biologically active agents, either sequentially, intermittently or in the same composition.

The most suitable route in any given case depends on a variety of factors, such as the nature of the disease, the tolerance of the subject to a particular administration route, the severity of the disease, and the particular composition that is used. Typically, the compositions provided herein are administered parenterally. In some examples, modified PH20 polypeptide compositions are administered so that they reach the interstitium of skin or tissues, thereby degrading the interstitial space for subsequent delivery of a therapeutic agent. Thus, in some examples, direct administration under the skin, such as by subcutaneous administration methods, is contemplated. Thus, in one example, local administration can be achieved by injection, such as from a syringe or other article of manufacture containing an injection device such as a needle. In another example, local administration can be achieved by infusion, which can be facilitated by the use of a pump or other similar device. Other modes of administration also are contemplated. For example, modified PH20 polypeptides, included conjugated forms with increased half-life such as PEGylated forms thereof, can be administered intravenously. Pharmaceutical compositions can be formulated in dosage forms appropriate for each route of administration.

Administration methods can be employed to decrease the exposure of selected modified PH20 polypeptides to degradative processes, such as proteolytic degradation and immunological intervention via antigenic and immunogenic responses. Examples of such methods include local administration at the site of treatment. PEGylation of therapeutics increases resistance to proteolysis, increases plasma half-life, and decreases antigenicity and immunogenicity. Examples of PEGylation methodologies are known in the art (see for example, Lu and Felix, Int. J. Peptide Protein Res., 43: 127-138, 1994; Lu and Felix, Peptide Res., 6: 140-6, 1993; Felix et al., Int. J. Peptide Res., 46: 253-64, 1995; Benhar et al., J. Biol. Chem., 269: 13398-404, 1994; Brumeanu et al., J Immunol, 154: 3088-95, 1995; see also, Caliceti et al. (2003) Adv. Drug Deliv. Rev. 55(10):1261-77 and Molineux (2003) Pharmacotherapy 23 (8 Pt 2):3S-8S). PEGylation also can be used in the delivery of nucleic acid molecules in vivo. For example, PEGylation of adenovirus can increase stability and gene transfer (see, e.g., Cheng et al. (2003) Pharm. Res. 20(9): 1444-51).

Various other delivery systems are known and can be used to administer selected PH20 polypeptides, such as but not limited to, encapsulation in liposomes, microparticles, microcapsules, recombinant cells capable of expressing the compound, receptor mediated endocytosis, and delivery of nucleic acid molecules encoding selected PH20 polypeptides such as retrovirus delivery systems.

Hence, in certain embodiments, liposomes and/or nanoparticles also can be employed with administration of soluble PH20 polypeptides. Liposomes are formed from phospholipids that are dispersed in an aqueous medium and spontaneously form multilamellar concentric bilayer vesicles (also termed multilamellar vesicles (MLVs)). MLVs generally have diameters of from 25 nm to 4 μm. Sonication of MLVs results in the formation of small unilamellar vesicles (SUVs) with diameters in the range of 200 to 500 angstroms containing an aqueous solution in the core.

Phospholipids can form a variety of structures other than liposomes when dispersed in water, depending on the molar ratio of lipid to water. At low ratios of lipid to water, liposomes form. Physical characteristics of liposomes depend on the pH, ionic strength and the presence of divalent cations. Liposomes can show low permeability to ionic and polar substances, but at elevated temperatures undergo a phase transition which markedly alters their permeability. The phase transition involves a change from a closely packed, ordered structure, known as the gel state, to a loosely packed, less-ordered structure, known as the fluid state. This occurs at a characteristic phase-transition temperature and results in an increase in permeability to ions, sugars and drugs.

Liposomes interact with cells via different mechanisms: endocytosis by phagocytic cells of the reticuloendothelial system such as macrophages and neutrophils; adsorption to the cell surface, either by nonspecific weak hydrophobic or electrostatic forces, or by specific interactions with cell-surface components; fusion with the plasma cell membrane by insertion of the lipid bilayer of the liposome into the plasma membrane, with simultaneous release of liposomal contents into the cytoplasm; and by transfer of liposomal lipids to cellular or subcellular membranes, or vice versa, without any association of the liposome contents. Varying the liposome formulation can alter which mechanism is operative, although more than one can operate at the same time. Nanocapsules can generally entrap compounds in a stable and reproducible way. To avoid side effects due to intracellular polymeric overloading, such ultrafine particles (sized around 0.1 μm) should be designed using polymers able to be degraded in vivo. Biodegradable polyalkyl-cyanoacrylate nanoparticles that meet these requirements are contemplated for use herein, and such particles can be easily made.

4. Combinations and Co-Formulations with Therapeutic Agents

Pharmaceutical compositions containing a modified PH20 polypeptide can be co-administered with another therapeutic agent. In such examples, the modified PH20 polypeptides can be formulated separately as a pharmaceutical composition and administered prior to, simultaneously with, intermittently with, or subsequent to a second composition containing an active therapeutic agent. In other examples, modified PH20 polypeptides can be co-formulated with pharmaceutical formulations of other therapeutic agents.

In particular, provided herein are co-formulations containing a modified PH20 polypeptide as described herein and a therapeutic agent that is a chemotherapeutic agent, an analgesic agent, an anti-inflammatory agent, an antimicrobial agent, an amoebicidal agent, a trichomonacidal agent, an anti-Parkinson agent, an anti-malarial agent, an anticonvulsant agent, an anti-depressant agent, and antiarthritics agent, an anti-fungal agent, an antihypertensive agent, an antipyretic agent, an anti-parasite agent, an antihistamine agent, an alpha-adrenergic agonist agent, an alpha blocker agent, an anesthetic agent, a bronchial dilator agent, a biocide agent, a bactericide agent, a bacteriostat agent, a beta adrenergic blocker agent, a calcium channel blocker agent, a cardiovascular drug agent, a contraceptive agent, a decongestant agent, a diuretic agent, a depressant agent, a diagnostic agent, an electrolyte agent, a hypnotic agent, a hormone agent, a hyperglycemic agent, a muscle relaxant agent, a muscle contractant agent, an ophthalmic agent, a parasympathomimetic agent, a psychic energizer agent, a sedative agent, a sympathomimetic agent, a tranquilizer agent, a urinary agent, a vaginal agent, a viricide agent, a vitamin agent, a non-steroidal anti-inflammatory agent, an angiotensin converting enzyme inhibitor agent, a polypeptide, a protein, a nucleic acid, a drug, an organic molecule or a sleep inducer. For example, modified PH20 polypeptides provided herein can be co-formulated with an antibody such as a monoclonal antibody, an Immune Globulin, an antibiotic, a bisphosphonate, a cytokine, a chemotherapeutic agent, a coagulation factor or an insulin. Exemplary therapeutic agents that can be co-formulated with a modified PH20 polypeptide are described in described in Section H.

5. Packaging, Articles of Manufacture and Kits

Pharmaceutical compounds of modified PH20 polypeptides, or nucleic acids encoding such polypeptides, or derivatives or variants thereof can be packaged as articles of manufacture containing packaging material, a pharmaceutical composition which is effective for treating a disease or disorder, and a label that indicates that the pharmaceutical composition or therapeutic molecule is to be used for treating the disease or disorder. Combinations of a selected modified PH20 polypeptide, or a derivative or variant thereof and an therapeutic agent also can be packaged in an article of manufacture. Typically, the modified PH20 polypeptides are packaged as systems for the non-refrigerated storage of the pharmaceutical compositions.

The articles of manufacture provided herein contain packaging materials. Packaging materials for use in packaging pharmaceutical products are well known to those of skill in the art. See, for example, U.S. Pat. Nos. 5,323,907, 5,052,558 and 5,033,252, each of which is incorporated herein in its entirety. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment. In particular, the container or other material is generally one that is suitable for storage without refrigeration, for example, is a syringe, tube, bottle, bag or vial. The articles of manufacture can include a needle or other injection device so as to facilitate administration (e.g., sub-epidermal administration) for local injection purposes. The choice of package depends on the PH20 and/or therapeutic agent, and whether such compositions will be packaged together or separately. In one example, the PH20 can be packaged as a mixture with the therapeutic agent. In another example, the components can be packaged as separate compositions

Modified PH20 polypeptides, therapeutic agents and/or articles of manufacture thereof also can be provided as kits. Kits can include a pharmaceutical composition described herein and an item for administration provided as an article of manufacture. For example a PH20 polypeptide can be supplied with a device for administration, such as a syringe, an inhaler, a dosage cup, a dropper, or an applicator. The compositions can be contained in the item for administration or can be provided separately to be added later. The kit can, optionally, include instructions for application including dosages, dosing regimens and instructions for modes of administration. Kits also can include a pharmaceutical composition described herein and an item for diagnosis. For example, such kits can include an item for measuring the concentration, amount or activity of the selected protease in a subject.

### G. METHODS OF ASSESSING PH20 ACTIVITY AND STABILITY

Assays can be used to assess the stability and activity of the PH20 polypeptides provided herein. The assays can be used to assess the hyaluronidase activity of the PH20 polypeptide under thermal stress conditions, including under various temperatures and/or over time. Other assays to assess stability also can be employed, such as assays to assess solubility, formation of aggregates, crystallization, oxidation and others within the knowledge of a skilled person.

1. Hyaluronidase Activity

The activity of a modified PH20 polypeptide can be assessed using methods well known in the art. For example, the USP XXII assay for hyaluronidase determines activity indirectly by measuring the amount of undegraded hyaluronic acid, or hyaluronan, (HA) substrate remaining after the enzyme is allowed to react with the HA for 30 min at 37° C. (USP XXII-NF XVII (1990) 644-645 United States Pharmacopeia Convention, Inc, Rockville, Md.). A Hyaluronidase Reference Standard (USP) or National Formulary (NF) Standard Hyaluronidase solution can be used in an assay to ascertain the activity, in units, of any hyaluronidase. In one example, activity is measured using a microturbidity assay. This is based on the formation of an insoluble precipitate when hyaluronic acid binds with a reagent that precipitates it, such as acidified serum or cetylpyridinium chloride (CPC). The activity is measured by incubating hyaluronidase with sodium hyaluronate (hyaluronic acid) for a set period of time (e.g., 10 minutes) and then precipitating the undigested sodium hyaluronate with the addition of acidified serum or CPC. The turbidity of the resulting sample is measured at 640 nm after an additional development period. The decrease in turbidity resulting from hyaluronidase activity on the sodium hyaluronate substrate is a measure of hyaluronidase enzymatic activity.

In another example, hyaluronidase activity is measured using a microtiter assay in which residual biotinylated hyaluronic acid is measured following incubation with hyaluronidase (see e.g., Frost and Stern (1997) Anal. Biochem. 251:263-269, U.S. Pat. Publication No. 20050260186). The free carboxyl groups on the glucuronic acid residues of hyaluronic acid are biotinylated, and the biotinylated hyaluronic acid substrate is covalently coupled to a microtiter plate. Following incubation with hyaluronidase, the residual biotinylated hyaluronic acid substrate is detected using an avidin-peroxidase reaction, and compared to that obtained following reaction with hyaluronidase standards of known activity.

Other assays to measure hyaluronidase activity also are known in the art and can be used in the methods herein (see e.g., Delpech et al., (1995) Anal. Biochem. 229:35-41; Takahashi et al., (2003) Anal. Biochem. 322:257-263).

Many hyaluronidase assays have been based upon the measurement of the generation of new reducing N-acetylamino groups (Bonner and Cantey, Clin. Chim Acta 13:746-752, 1966), or loss of viscosity (De Salegui et al., Arch. Biochem. Biophys. 121:548-554, 1967) or turbidity (Dorfman and Ott, J. Biol. Chem. 172:367, 1948). With purified substrates all of these methods suffice for determination of the presence or absence of endoglycosidase activity.

Substantially purified glycosaminoglycan substrates can also be used in a Gel Shift Assay. Glycosaminoglycans are mixed with recombinant PH20, such as a soluble PH20, to test for endoglycosidase activity that results in a shift in substrate mobility within the gel. Examples of such substrates include, but are not limited to, chondroitin-4 and 6 sulfate, dermatan sulfate, heparan-sulfate, which can be obtained from Sigma Chemical. Human umbilical cord Hyaluronan can be obtained from ICN. For example, each test substrate can be diluted to at or about 0.1 mg/mL in a buffer range from pH 3.5-7.5. In such an exemplary assay, at or about 10 μl samples of purified soluble PH20 or conditioned media from PH20 expressing cells can be mixed with at or about 90 μl of test substrate in desired buffer and incubated for 3 hours at 37° C. Following incubation, samples are neutralized with sample buffer (Tris EDTA pH 8.0, Bromophenol Blue and glycerol) followed by electrophoresis. Glycosaminoglycans can be detected using any method known in the art, for example, glycosaminoglycans can be detected by staining the gels using 0.5% Alcian Blue in 3% Glacial Acetic Acid overnight followed by destaining in 7% Glacial Acetic Acid. Degradation is determined by comparison of substrate mobility in the presence and absence of enzyme.

Hyaluronidase activity can also be detected by substrate gel zymography (Guentenhoner et al. (1992) Matrix 12:388-396). In this assay, a sample is applied to an SDS-PAGE gel containing hyaluronic acid and the proteins in the sample separated by electrophoresis. The gel is then incubated in an enzyme assay buffer and subsequently stained to detect the hyaluronic acid in the gel. Hyaluronidase activity is visualized as a cleared zone in the substrate gel.

The ability of a PH20 polypeptide, including a modified PH20 polypeptide provided herein, to act as a spreading or diffusing agent also can be assessed. For example, trypan blue dye can be injected subcutaneously with or without a PH20 polypeptide into the lateral skin on each side of nude mice. The dye area is then measured, such as with a microcaliper, to determine the ability of the PH20 polypeptide to act as a spreading agent (U.S. Pat. Pub. No. 20060104968).

The functional activity of a PH20 polypeptide can be compared and/or normalized to a reference standard using any of these assays. This can be done to determine what a functionally equivalent amount of a PH20 polypeptide is. For example, the ability of a PH20 polypeptide to act as a spreading or diffusing agent can be assessed by injecting it into the lateral skin of mice with trypan blue, and the amount required to achieve the same amount of diffusion as, for example, 100 units of a Hyaluronidase Reference Standard, can be determined. The amount of PH20 polypeptide required is, therefore, functionally equivalent to 100 hyaluronidase units.

2. Thermal Stability

The stability of a protein can be determined by measuring the activity of the protein as a function of time. The unfolding temperature (Tm) of the protein can be used as a marker of solution stability and in vivo stability for proteins. The unfolding temperature of a particular protein refers to that temperature at which the protein loses its secondary structure and typically, its activity and can be determined using methods known to those of skill in the art, such as differential scanning calorimetry.

For example, thermal stability can be analyzed using a number of non-limiting biophysical or biochemical techniques known in the art. In particular examples, thermal stability is evaluated by analytical spectroscopy. An exemplary analytical spectroscopy method is Differential Scanning calorimetry (DSC). DSC employs a calorimeter which is sensitive to the heat absorbances that accompany the unfolding of most proteins or protein domains (see, e.g. Sanchez-Ruiz, et al., Biochemistry, 27: 1648-52, 1988). To determine the thermal stability of a protein, a sample of the protein is inserted into the calorimeter and the temperature is raised until the protein unfolds. The temperature at which the protein unfolds is indicative of overall protein stability.

Another exemplary analytical spectroscopy method is Circular Dichroism (CD) spectroscopy. CD spectrometry measures the optical activity of a composition as a function of increasing temperature. Circular dichroism (CD) spectroscopy measures differences in the absorption of left-handed polarized light versus right-handed polarized light which arise due to structural asymmetry. A disordered or unfolded structure results in a CD spectrum very different from that of an ordered or folded structure. The CD spectrum reflects the sensitivity of the proteins to the denaturing effects of increasing temperature and is therefore indicative of a protein's thermal stability (see van Mierlo and Steemsma, J. Biotechnol., 79(3):281-98, 2000).

Another exemplary analytical spectroscopy method for measuring thermal stability is Fluorescence Emission Spectroscopy (see van Mierlo and Steemsma, supra). Yet another exemplary analytical spectroscopy method for measuring thermal stability is Nuclear Magnetic Resonance (NMR) spectroscopy (see, e.g. van Mierlo and Steemsma, supra).

In certain embodiments, thermal stability is evaluated by measuring the melting temperature (Tm) of a polypeptide composition using any of the above techniques (e.g. analytical spectroscopy techniques). The melting temperature is the temperature at the midpoint of a thermal transition curve wherein 50% of molecules of a composition are in a folded state. In one example, the melting temperature of a PH20 polypeptide, such as a modified PH20 polypeptide, can be determined by measuring the hydrodynamic radius of particles by dynamic light scattering under various conditions (e.g., various temperatures over time). An increase in particle size and a decrease in the melting temperature indicates denaturation and subsequent aggregation of the hyaluronidase.

In other embodiments, the thermal stability can be measured biochemically. An exemplary biochemical method for assessing thermal stability is a thermal challenge assay. In a “thermal challenge assay,” a polypeptide is subjected to a range of elevated temperatures for a set period of time. For example, in one embodiment, test polypeptides are subject to a range of increasing temperatures, e.g., for 10 minutes. The activity of the protein is then assayed by a relevant biochemical assay (e.g. hyaluronidase assay). The thermal challenge assay can be used to determine the temperature at which 50% hyaluronidase activity is retained (i.e. the T_{C} value or T₅₀). The Tc or T₅₀ values are not necessarily equivalent to the biophysically derived Tₘ values. Such an assay can be done in a high-throughput format. For example, a library of modified hyaluronan-degrading enzymes (e.g. modified PH20 polypeptides) can be created using methods known in the art. The modified polypeptide(s) can be subjected to thermal challenge. The challenged test samples can be assayed for hyaluronidase activity and those that are stable can be scaled up and further characterized.

3. Other Assays to Assess Stability

The stability of a PH20 polypeptide provided herein also can be assessed using other methods and assays known in the art, such as assays that assess purity, recovery, crystallization or aggregation. For example, in addition to assessing stability based on hyaluronidase activity, stability can be assessed by visual inspection, percent recovery, protein purity and apparent melting temperature.

For example, protein purity can be measured by reversed phase high performance liquid chromatography (RP-HPLC). Protein purity, as determined by RP-HPLC, is the percent of the main PH20 protein peak present, as compared to all of the protein species present. Thus, RP-HPLC, and similar methods known to one of skill in the art, can assess degradation of the enzyme. Protein purity can be assessed over time. Percent recovery also can be determined as the relative percentage of the polypeptide under the thermal stress condition (e.g. 52° C. for 10 minutes) as compared to a reference sample or the same polypeptide under thermal neutral conditions (e.g. 4° C. for 10 minutes). PH20 polypeptide stability also can be determined by measuring the oxidation of the hyaluronidase by RP-HPLC. Percent oxidation is a measure of sum of the peak areas of the major (ox-1) and minor (ox-2) peaks.

Other methods known to one of skill in the art that can be used to determine the stability of the hyaluronidase in the co-formulations provided herein, include polyacrylamide gel electrophoresis (PAGE), immunoblotting, nuclear magnetic resonance (NMR) spectroscopy, mass spectrometry, circular dichroism (CD) and dye-based fluorescence assays.

4. Solubility

The solubility of a PH20 polypeptide can be determined by any method known to one of the skill in the art. One method for determining solubility is detergent partitioning. For example, a soluble PH20 polypeptide can be distinguished, for example, by its partitioning into the aqueous phase of a Triton® X-114 solution at 37° C. (Bordier et al., (1981) J. Biol. Chem., 256:1604-1607). Membrane-anchored polypeptides, such as lipid-anchored hyaluronidases, including GPI-anchored hyaluronidases, will partition into the detergent-rich phase, but will partition into the detergent-poor or aqueous phase following treatment with Phospholipase C. Phospholipase C is an enzyme that cleaves the phospho-glycerol bond found in GPI-anchored proteins. Treatment with PLC will cause release of GPI-linked proteins from the outer cell membrane.

5. Pharmacodynamics/Pharmacokinetics

The pharmacokinetic and pharmacodynamic properties of a PH20 polypeptide, such as a modified PH20 polypeptide, alone or in combination with another therapeutic agent, also can be assessed in vivo using animal models and/or human subjects, such as in the setting of a clinical trial. Pharmacokinetic or pharmacodynamic studies can be performed using animal models or can be performed during studies with patients administered with a PH20 polypeptide or modified PH20 polypeptide.

Animal models include, but are not limited to, mice, rats, rabbits, dogs, guinea pigs and non-human primate models, such as cynomolgus monkeys or rhesus macaques. In some instances, pharmacokinetic or pharmacodynamic studies are performed using healthy animals. In other examples, the studies are performed using animal models of a disease for which therapy with hyaluronan is considered, such as animal models of any hyaluronan-associated disease or disorder, for example a tumor model.

The pharmacokinetic properties of a PH20 polypeptide, such as a modified PH20 polypeptide, can be assessed by measuring such parameters as the maximum (peak) concentration (Cₘₐₓ), the peak time (i.e., when maximum concentration occurs; Tₘₐₓ), the minimum concentration (i.e., the minimum concentration between doses; Cₘᵢₙ), the elimination half-life (T_{1/2}) and area under the curve (i.e., the area under the curve generated by plotting time versus concentration; AUC), following administration. The absolute bioavailability of the hyaluronidase can be determined by comparing the area under the curve of hyaluronidase following subcutaneous delivery (AUC_{sc}) with the AUC of hyaluronidase following intravenous delivery (AUCᵢᵥ). Absolute bioavailability (F), can be calculated using the formula: F=([AUC]_{sc}×dose_{sc})/([AUC]ᵢᵥ×doseᵢᵥ). A range of doses and different dosing frequency of dosing can be administered in the pharmacokinetic studies to assess the effect of increasing or decreasing concentrations enzyme, such as modified PH20 polypeptide, in the dose.

### H. METHODS OF TREATMENT AND COMBINATION THERAPY

Provided herein are methods and uses of any of the modified PH20 polypeptides provided herein that exhibit hyaluronidase activity based on its ability to degrade glycosaminoglycan(s) such as hyaluronan. Due to such activity, the modified PH20 polypeptides can be used as a spreading factor to increase the delivery and/or bioavailability of subcutaneously administered therapeutic agents. Delivery of any therapeutic agent, including but not limited to, peptides, proteins, small molecule drugs, nucleic acids, or viruses can be facilitated or enhanced by co-administration with a modified PH20 polypeptide provided herein. For example, modified PH20 polypeptides can be used to increase the delivery of therapeutic agents such as antibodies (e.g., monoclonal antibodies), cytokines, Immune Globulin, an Insulin, or coagulation factors, to a desired locus, such as by increasing penetration of chemotherapeutic agents into solid tumors. The modified PH20 polypeptides also can be used to treat a hyaluronan-disease or disorder that is characterized by an excess or accumulation of hyaluronan. For example, modified PH20 polypeptides provided herein can be used to for treating a tumor; for treating glycosaminoglycan accumulation in the brain; for treating a cardiovascular disorder; for treating an ophthalmic disorder; for treating pulmonary disease; for treating cellulite; and/or for treating a proliferative disorder.

Other methods and uses of a modified PH20 polypeptide include any that are known to one of skill in the art. For example, various forms of PH20 hyaluronidases have been prepared and approved for therapeutic use in humans. For example, animal-derived hyaluronidase preparations include Vitrase® (ISTA Pharmaceuticals), a purified ovine testicular hyaluronidase, and Amphadase® (Amphastar Pharmaceuticals), a bovine testicular hyaluronidase. Hylenex® (Halozyme Therapeutics) is a human recombinant hyaluronidase produced by genetically engineered Chinese Hamster Ovary (CHO) cells containing nucleic acid encoding for soluble rHuPH20 (see e.g., U.S. Pat. No. 7,767,429). Approved therapeutic uses for hyaluronidases include use as an adjuvant to increase the absorption and dispersion of other therapeutic agents for hypodermoclysis (subcutaneous fluid administration), and as an adjunct in subcutaneous urography for improving resorption of radiopaque agents. In addition to these indications, hyaluronidases can be used as a therapeutic or cosmetic agent for the treatment of additional diseases and conditions. For example, hyaluronidase is commonly used, for example, for peribulbar block in local anesthesia prior ophthalmic surgery. The presence of the enzyme prevents the need for additional blocks and reduces the time to the onset of akinesia (loss of eye movement). Peribulbar and sub-Tenon's block are the most common applications of hyaluronidase for ophthalmic procedures. Hyaluronidase also can promote akinesia in cosmetic surgery, such as blepharoplasties and face lifts. It is understood that modified PH20 hyaluronidases provided herein can be used in any method of treatment or combination therapy for which a PH20 hyaluronidase is used (see e.g., U.S. Publication Nos. US20040268425; US20050260186; US20060104968; and U.S. application Ser. No. 12/381,844, published as U.S. Publication No. US20100074885; Ser. No. 12/386,249, published as U.S. Publication No. US20090311237; Ser. No. 12/387,225, published as U.S. Publication No. US20090304665; and Ser. No. 12/386,222, published as U.S. Publication No. US2010003238, each incorporated by reference in their entirety).

Exemplary, non-limiting, methods and uses are described in the following subsections.

1. Methods of Delivering Therapeutic Agents

As noted above, hyaluronidase is a spreading or diffusing substance that modifies the permeability of connective tissue through the hydrolysis of hyaluronic acid, a polysaccharide found in the intercellular ground substance of connective tissue, and of certain specialized tissues, such as the umbilical cord and vitreous humor. When no spreading factor is present, materials injected subcutaneously, such as drugs, proteins, peptides and nucleic acid, spread very slowly. Co-injection with hyaluronidase, however, can cause rapid spreading. The rate of diffusion is proportional to the amount of enzyme, and the extent of diffusion is proportional to the volume of solution.

Any modified PH20 polypeptides provided herein can be used to promote or enhance the delivery agents and molecules to any of a variety of mammalian tissues in vivo. It can be used to facilitate the diffusion and, therefore, promote the delivery, of small molecule pharmacologic agents as well as larger molecule pharmacologic agents, such as proteins, nucleic acids and ribonucleic acids, and macromolecular compositions than can contain a combination of components including, but not limited to, nucleic acids, proteins, carbohydrates, lipids, lipid-based molecules and drugs (see e.g., U.S. Publication Nos. US20040268425; US20050260186; and US20060104968). Any of the modified PH20 polypeptides can be co-administered and/or co-formulated with a therapeutic agent to improve the bioavailability as well as pharmacokinetic (PK) and/or pharmacodynamic (PD) characteristics of the co-formulated or co-administered agent. PK/PD parameters that can be improved by using co-administering a therapeutic agent with a soluble PH20, such as a modified PH20 provided herein, include such measures as Cₘₐₓ (the maximal concentration of agent achieved following absorption in, e.g., the bloodstream), Tₘₐₓ (the time required to achieve maximal concentration), T_{1/2} (the time required for the concentration to fall by half), Cₘᵢₙ (the minimal concentration of agent following metabolism and excretion), AUC (area under the curve of concentration versus time, a measure of the overall amount of bioavailability), concentrations in various tissues of interest (including, e.g., the rate of achieving desired concentrations, the overall levels, and the duration of maintaining desired levels), and Eₘₐₓ (the maximal effect achieved).

Thus, the methods of treatment provided herein include combination therapies in which any of the modified PH20 polypeptides are co-administered with a therapeutic agent for the treatment of a disease or disorder for which the therapeutic agent treats. Any therapeutic agent that ameliorates and or otherwise lessens the severity of a disease or condition can be combined with a modified PH20 polypeptide provided herein in order to increase the bioavailability of such therapeutic agent. In particular, modified PH20 polypeptides provided herein can be used in each and all of the combinations described in applications see e.g., U.S. Publication Nos. US20040268425; US20050260186; US20060104968 and U.S. application Ser. No. 12/381,844, published as U.S. Publication No. US20100074885; Ser. No. 12/386,249, published as U.S. Publication No. US20090311237; Ser. No. 12/387,225, published as U.S. Publication No. US20090304665; and Ser. No. 12/386,222, published as U.S. Publication No. US2010003238 in place of the disclosed hyaluronidase or hyaluronidase degrading enzyme.

Modified PH20 polypeptides can be administered prior to, subsequent to, intermittently with or simultaneously with the therapeutic agent preparation. Generally, the modified PH20 polypeptide is administered prior to or simultaneously with administration of the therapeutic agent preparation to permit the PH20 to degrade the hyaluronic acid in the interstitial space. The PH20 can be administered at a site different from the site of administration of the therapeutic molecule or the soluble PH20 can be administered at a site the same as the site of administration of the therapeutic molecule.

Examples of pharmaceutical, therapeutic and cosmetic agents and molecules that can be administered with hyaluronidase include, but are not limited to, a chemotherapeutic or anticancer agent, an analgesic agent, an antibiotic agent, an anti-inflammatory agent, an antimicrobial agent, an amoebicidal agent, a trichomonacidal agent, an anti-Parkinson agent, an anti-malarial agent, an anticonvulsant agent, an anti-depressant agent, an anti-arthritic agent, an anti-fungal agent, an antihypertensive agent, an antipyretic agent, an anti-parasitic agent, an antihistamine agent, an alpha-adrenergic agonist agent, an alpha blocker agent, an anesthetic agent, a bronchial dilator agent, a biocide agent, a bactericide agent, a bacteriostatic agent, a beta adrenergic blocker agent, a calcium channel blocker agent, a cardiovascular drug agent, a contraceptive agent, a cosmetic or esthetic agent, a decongestant agent, a diuretic agent, a depressant agent, a diagnostic agent, an electrolyte agent, a hypnotic agent, a hormone agent, a hyperglycemic agent, a muscle relaxant agent, a muscle contractant agent, an ophthalmic agent, a parasympathomimetic agent, a psychic energizer agent, a sedative agent, a sleep inducer, a sympathomimetic agent, a tranquilizer agent, a urinary agent, a vaginal agent, a viricide agent, a vitamin agent, a non-steroidal anti-inflammatory agent, or an angiotensin converting enzyme inhibitor agent, and any combination thereof. In particular, therapeutic agents include antibodies, including monoclonal antibodies, bisphosphonates, insulins, coagulation factors, cytokines and Immune Globulins.

For example, modified PH20 polypeptides provided herein can be used to increase the delivery of chemotherapeutic agents. Hyaluronidases have also been used to enhance the activity of chemotherapeutics and/or the accessibility of tumors to chemotherapeutics (Schuller et al., 1991, Proc. Amer. Assoc. Cancer Res. 32:173, abstract no. 1034; Czejka et al., 1990, Pharmazie 45:H.9; Baumgartner et al. (1988) Reg. Cancer Treat. 1:55-58; Zanker et al. (1986) Proc. Amer. Assoc. Cancer Res. 27:390). Combination chemotherapy with hyaluronidase is effective in the treatment of a variety of cancers including urinary bladder cancer (Horn et al., 1985, J Surg. Oncol. 28:304-307), squamous cell carcinoma (Kohno et al., 94, J. Cancer Res. Oncol. 120:293-297), breast cancer (Beckenlehner et al., 1992, J. Cancer Res. Oncol. 118:591-596), and gastrointestinal cancer (Scheithauer et al., 1988, Anticancer Res. 8:391-396), prostate cancer, pancreatic cancer and other cancers. In this example, the modified PH20 hyaluronidase enhances penetration of chemotherapeutic or other anti-cancer agents into solid tumors, thereby treating the disease.

Compositions containing soluble PH20 can be injected intratumorally with anti-cancer agents or intravenously for disseminated cancers or hard to reach tumors. The anticancer agent can be a chemotherapeutic, an antibody, a peptide, or a gene therapy vector, virus or DNA. Additionally, hyaluronidase can be used to recruit tumor cells into the cycling pool for sensitization in previously chemorefractory tumors that have acquired multiple drug resistance (St Croix et al., (1998) Cancer Lett September 131(1): 35-44).

Exemplary anti-cancer agents that can be administered after, coincident with or before administration of a modified PH20 polypeptide provided herein, include, but are not limited to Acivicins; Aclarubicins; Acodazoles; Acronines; Adozelesins; Aldesleukins; Alemtuzumabs; Alitretinoins (9-Cis-Retinoic Acids); Allopurinols; Altretamines; Alvocidibs; Ambazones; Ambomycins; Ametantrones; Amifostines; Aminoglutethimides; Amsacrines; Anastrozoles; Anaxirones; Ancitabines; Anthramycins; Apaziquones; Argimesnas; Arsenic Trioxides; Asparaginases; Asperlins; Atrimustines; Azacitidines; Azetepas; Azotomycins; Banoxantrones; Batabulins; Batimastats; BCG Live; Benaxibines; Bendamustines; Benzodepas; Bexarotenes; Bevacizumab; Bicalutamides; Bietaserpines; Biricodars; Bisantrenes; Bisnafide Dimesylates; Bizelesins; Bleomycins; Bortezomibs; Brequinars; Bropirimines; Budotitanes; Busulfans; Cactinomycins; Calusterones; Canertinibs; Capecitabines; Caracemides; Carbetimers; Carboplatins; Carboquones; Carmofurs; Carmustines with Polifeprosans; Carmustines; Carubicins; Carzelesins; Cedefingols; Celecoxibs; Cemadotins; Chlorambucils; Cioteronels; Ciplactin; Cirolemycins; Cisplatins; Cladribines; Clanfenurs; Clofarabines; Crisnatols; Cyclophosphamides; Cytarabine liposomals; Cytarabines; Dacarbazines; Dactinomycins; Darbepoetin Alfas; Daunorubicin liposomals; Daunorubicins/Daunomycins; Daunorubicins; Decitabines; Denileukin Diftitoxes; Dexniguldipines; Dexonas; Dexrazoxanes; Dezaguanines; Diaziquones; Dibrospidiums; Dienogests; Dinalins; Disermolides; Docetaxels; Dofequidars; Doxifluridines; Doxorubicin liposomals; Doxorubicin HCl; Doxorubicin HCl liposome injection; Doxorubicins; Droloxifenes; Dromostanolone Propionates; Duazomycins; Ecomustines; Edatrexates; Edotecarins; Eflornithines; Elacridars; Elinafides; Elliott's B Solutions; Elsamitrucins; Emitefurs; Enloplatins; Enpromates; Enzastaurins; Epipropidines; Epirubicins; Epoetin alfas; Eptaloprosts; Erbulozoles; Esorubicins; Estramustines; Etanidazoles; Etoglucids; Etoposide phosphates; Etoposide VP-16s; Etoposides; Etoprines; Exemestanes; Exisulinds; Fadrozoles; Fazarabines; Fenretinides; Filgrastims; Floxuridines; Fludarabines; Fluorouracils; 5-fluorouracils; Fluoxymesterones; Flurocitabines; Fosquidones; Fostriecins; Fostriecins; Fotretamines; Fulvestrants; Galarubicins; Galocitabines; Gemcitabines; Gemtuzumabs/Ozogamicins; Geroquinols; Gimatecans; Gimeracils; Gloxazones; Glufosfamides; Goserelin acetates; Hydroxyureas; Ibritumomabs/Tiuxetans; Idarubicins; Ifosfamides; Ilmofosines; Ilomastats; Imatinib mesylates; Imexons; Improsulfans; Indisulams; Inproquones; Interferon alfa-2as; Interferon alfa-2bs; Interferon Alfas; Interferon Betas; Interferon Gammas; Interferons; Interleukin-2s and other Interleukins (including recombinant Interleukins); Intoplicines; Iobenguanes [131-I]; Iproplatins; Irinotecans; Irsogladines; Ixabepilones; Ketotrexates; L-Alanosines; Lanreotides; Lapatinibs; Ledoxantrones; Letrozoles; Leucovorins; Leuprolides; Leuprorelins (Leuprolides); Levamisoles; Lexacalcitols; Liarozoles; Lobaplatins; Lometrexols; Lomustines/CCNUs; Lomustines; Lonafarnibs; Losoxantrones; Lurtotecans; Mafosfamides; Mannosulfans; Marimastats; Masoprocols; Maytansines; Mechlorethamines; Mechlorethamines/Nitrogen mustards; Megestrol acetates; Megestrols; Melengestrols; Melphalans; Melphalan L-PAMs; Menogarils; Mepitiostanes; Mercaptopurines; 6-Mecaptopurine; Mesnas; Metesinds; Methotrexates; Methoxsalens; Metomidates; Metoprines; Meturedepas; Miboplatins; Miproxifenes; Misonidazoles; Mitindomides; Mitocarcins; Mitocromins; Mitoflaxones; Mitogillins; Mitoguazones; Mitomalcins; Mitomycin Cs; Mitomycins; Mitonafides; Mitoquidones; Mitospers; Mitotanes; Mitoxantrones; Mitozolomides; Mivobulins; Mizoribines; Mofarotenes; Mopidamols; Mubritinibs; Mycophenolic Acids; Nandrolone Phenpropionates; Nedaplatins; Nelarabines; Nemorubicins; Nitracrines; Nocodazoles; Nofetumomabs; Nogalamycins; Nolatrexeds; Nortopixantrones; Octreotides; Oprelvekins; Ormaplatins; Ortataxels; Oteracils; Oxaliplatins; Oxisurans; Oxophenarsines; Paclitaxels; Pamidronates; Patupilones; Pegademases; Pegaspargases; Pegfilgrastims; Peldesines; Peliomycins; Pelitrexols; Pemetrexeds; Pentamustines; Pentostatins; Peplomycins; Perfosfamides; Perifosines; Picoplatins; Pinafides; Pipobromans; Piposulfans; Pirfenidones; Piroxantrones; Pixantrones; Plevitrexeds; Plicamycin Mithramycins; Plicamycins; Plomestanes; Plomestanes; Porfimer sodiums; Porfimers; Porfiromycins; Prednimustines; Procarbazines; Propamidines; Prospidiums; Pumitepas; Puromycins; Pyrazofurins; Quinacrines; Ranimustines; Rasburicases; Riboprines; Ritrosulfans; Rituximabs; Rogletimides; Roquinimexs; Rufocromomycins; Sabarubicins; Safingols; Sargramostims; Satraplatins; Sebriplatins; Semustines; Simtrazenes; Sizofirans; Sobuzoxanes; Sorafenibs; Sparfosates; Sparfosic Acids; Sparsomycins; Spirogermaniums; Spiromustines; Spiroplatins; Spiroplatins; Squalamines; Streptonigrins; Streptovarycins; Streptozocins; Sufosfamides; Sulofenurs; Sunitinib Malate; 6-TG; Tacedinalines; Talcs; Talisomycins; Tallimustines; Tamoxifens; Tariquidars; Tauromustines; Tecogalans; Tegafurs; Teloxantrones; Temoporfins; Temozolomides; Teniposides/VM-26s; Teniposides; Teroxirones; Testolactones; Thiamiprines; Thioguanines; Thiotepas; Tiamiprines; Tiazofurins; Tilomisoles; Tilorones; Timcodars; Timonacics; Tirapazamines; Topixantrones; Topotecans; Toremifenes; Tositumomabs; Trabectedins (Ecteinascidin 743); Trastuzumabs; Trestolones; Tretinoins/ATRA; Triciribines; Trilostanes; Trimetrexates; Triplatin Tetranitrates; Triptorelins; Trofosfamides; Tubulozoles; Ubenimexs; Uracil Mustards; Uredepas; Valrubicins; Valspodars; Vapreotides; Verteporfins; Vinblastines; Vincristines; Vindesines; Vinepidines; Vinflunines; Vinformides; Vinglycinates; Vinleucinols; Vinleurosines; Vinorelbines; Vinrosidines; Vintriptols; Vinzolidines; Vorozoles; Xanthomycin A's (Guamecyclines); Zeniplatins; Zilascorbs [2-H]; Zinostatins; Zoledronate; Zorubicins; and Zosuquidars, for example:

Aldesleukins (e.g., PROLEUKIN®); Alemtuzumabs (e.g., CAMPATH®); Alitretinoins (e.g., PANRETIN®); Allopurinols (e.g., ZYLOPRIM®); Altretamines (e.g., HEXALEN®); Amifostines (e.g., ETHYOL®); Anastrozoles (e.g., ARIMIDEX®); Arsenic Trioxides (e.g., TRISENOX®); Asparaginases (e.g., ELSPAR®); BCG Live (e.g., TICE® BCG); Bexarotenes (e.g., TARGRETIN®); Bevacizumab (AVASTIN®); Bleomycins (e.g., BLENOXANE®); Busulfan intravenous (e.g., BUSULFEX®); Busulfan orals (e.g., MYLERAN™); Calusterones (e.g., METHOSARB®); Capecitabines (e.g., XELODA®); Carboplatins (e.g., PARAPLATIN®); Carmustines (e.g., BCNUO, BiCNU®); Carmustines with Polifeprosans (e.g., GLIADEL® Wafer); Celecoxibs (e.g., CELEBREX®); Chlorambucils (e.g., LEUKERAN®); Cisplatins (e.g., PLATINOL®); Cladribines (e.g., LEUSTATIN®, 2-CdA®); Cyclophosphamides (e.g., CYTOXAN®, NEOSAR®); Cytarabines (e.g., CYTOSAR-U®); Cytarabine liposomals (e.g., DepoCyt®); Dacarbazines (e.g., DTIC-Domeυ): Dactinomycins (e.g., COSMEGEN®); Darbepoetin Alfas (e.g., ARANESP®); Daunorubicin liposomals (e.g. DAUNOXOME®); Daunorubicins/Daunomycins (e.g., CERUBIDINE®); Denileukin Diftitoxes (e.g., ONTAK®); Dexrazoxanes (e.g., ZINECARD®); Docetaxels (e.g., TAXOTERE®); Doxorubicins (e.g., ADRIAMYCINO, RUBEX®); Doxorubicin liposomals, including Doxorubicin HCl liposome injections (e.g., DOXIL®); Dromostanolone propionates (e.g., DROMOSTANOLONE® and MASTERONE® Injection); Elliott's B Solutions (e.g., Elliott's B Solution®); Epirubicins (e.g., ELLENCE®); Epoetin alfas (e.g., EPOGEN®); Estramustines (e.g., EMCYT®); Etoposide phosphates (e.g., ETOPOPHOS®); Etoposide VP-16s (e.g., VEPESID®); Exemestanes (e.g., AROMASIN®); Filgrastims (e.g., NEUPOGEN®); Floxuridines (e.g., FUDR®); Fludarabines (e.g., FLUDARA®); Fluorouracils incl. 5-FUs (e.g., ADRUCIL®); Fulvestrants (e.g., FASLODEX®); Gemcitabines (e.g., GEMZAR®); Gemtuzumabs/Ozogamicins (e.g., MYLOTARG®); Goserelin acetates (e.g., ZOLADEX®); Hydroxyureas (e.g., HYDREA®); Ibritumomabs/Tiuxetans (e.g., ZEVALIN®); Idarubicins (e.g., IDAMYCIN®); Ifosfamides (e.g., IFEX®); Imatinib mesylates (e.g., GLEEVEC®); Interferon alfa-2as (e.g., ROFERON-A®); Interferon alfa-2bs (e.g., INTRON A®); Irinotecans (e.g., CAMPTOSAR®); Letrozoles (e.g., FEMARA®); Leucovorins (e.g., WELLCOVORIN®, LEUCOVORIN®); Levamisoles (e.g., ERGAMISOL®); Lomustines/CCNUs (e.g., CeeNU®); Mechlorethamines/Nitrogen mustards (e.g., MUSTARGEN®); Megestrol acetates (e.g., MEGACE®); Melphalans/L-PAMs (e.g., ALKERAN®); Mercaptopurine incl. 6-MPs (e.g., PURINETHOL®); Mesnas (e.g., MESNEX®); Methotrexates; Methoxsalens (e.g., UVADEX®); Mitomycin Cs (e.g., MUTAMYCIN®, MITOZYTREX®); Mitotanes (e.g., LYSODREN®); Mitoxantrones (e.g., NOVANTRONE®); Nandrolone Phenpropionates (e.g., DURABOLIN-50®); Nofetumomabs (e.g., VERLUMA®); Oprelvekins (e.g., NEUMEGA®); Oxaliplatins (e.g., ELOXATIN®); Paclitaxels (e.g., PAXENE®, TAXOL®); Pamidronates (e.g., AREDIA®); Pegademases (e.g., ADAGEN®); Pegaspargases (e.g., ONCASPAR®); Pegfilgrastims (e.g., NEULASTA®); Pentostatins (e.g., NIPENT®); Pipobromans (e.g., VERCYTE®); Plicamycin/Mithramycins (e.g., MITHRACIN®); Porfimer sodiums (e.g., PHOTOFRIN®); Procarbazines (e.g., MATULANE®); Quinacrines (e.g., ATABRINE®); Rasburicases (e.g., ELITEK®); Rituximabs (e.g., RITUXAN®); Sargramostims (e.g., PROKINE®); Streptozocins (e.g., ZANOSAR®); Sunitinib Malates (e.g., SUTENT®); Talcs (e.g., SCLEROSOL®); Tamoxifens (e.g., NOLVADEX®); Temozolomides (e.g., TEMODAR®); Teniposides/VM-26s (e.g., VUMON®); Testolactones (e.g., TESLAC®); Thioguanines incl. 6-TG; Thiotepas (e.g., THIOPLEX®); Topotecans (e.g., HYCAMTIN®); Toremifenes (e.g., FARESTON®); Tositumomabs (e.g., BEXXAR®); Trastuzumabs (e.g., HERCEPTIN®); Tretinoins/ATRA (e.g., VESANOID®); Uracil Mustards; Valrubicins (e.g., VALSTAR®); Vinblastines (e.g., VELBAN®); Vincristines (e.g., ONCOVIN®); Vinorelbines (e.g., NAVELBINE®); and Zoledronates (e.g., ZOMETA®).

For example, exemplary antibiotic agents include, but are not limited to, Aminoglycosides; Amphenicols; Ansamycins; Carbacephems; Carbapenems; Cephalosporins or Cephems; Cephamycins; Clavams; Cyclic lipopeptides; Diaminopyrimidines; Ketolides; Lincosamides; Macrolides; Monobactams; Nitrofurans; Oxacephems; Oxazolidinones; Penems, thienamycins and miscellaneous beta-lactams; Penicillins; Polypeptides antibiotics; Quinolones; Sulfonamides; Sulfones; Tetracyclines; and other antibiotics (such as Clofoctols, Fusidic acids, Hexedines, Methenamines, Nitrofurantoins Nitroxolines, Ritipenems, Taurolidines, Xibomols).

Also included among exemplary therapeutic agents are coagulation factors or other blood modifiers such as antihemophilic factors, anti-inhibitor coagulant complexes, antithrombin III, coagulation Factor V, coagulation Factor VIII, coagulation Factor IX, plasma protein fractions, von Willebrand factors; antiplatelet agents (including, for example, abciximabs, anagrelides, cilostazols, clopidogrel bisulfates, dipyridamoles, epoprostenols, eptifibatides, tirofibans; colony stimulating factors (CSFs) (including, for example, Granulocyte CSFs and Granulocyte Macrophage CSFs); erythropoiesis stimulators (including, for example, erythropoietins such as darbepoetin alfas) and epoetin alfas; hemostatics and albumins (including, for example, aprotinins, combinations of antihemophilic factors and plasma, Desmopressin Acetates, and albumins); immune globulins, as well as hepatitis B immune globulins; thrombin inhibitors (including for example direct thrombin inhibitors and lepirudin), and drotrecogin alfas; anticoagulants (including, for example, dalteparins, enoxaparins and other heparins, and warfarins).

Exemplary antibodies or other therapeutic agents include, but are not limited to, Cetuximab (IMC-C225; Erbitux®); Trastuzumab (Herceptin®); Rituximab (Rituxan®; MabThera®); Bevacizumab (Avastin®); Alemtuzumab (Campath®; Campath-1H®; Mabcampath®); Panitumumab (ABX-EGF; Vectibix®); Ranibizumab (Lucentis®); Ibritumomab; Ibritumomab tiuxetan (Zevalin®); Tositumomab; Iodine I 131 Tositumomab (BEXXAR®); Catumaxomab (Removab®); Gemtuzumab; Gemtuzumab ozogamicin (Mylotarg®); Abatacept (CTLA4-Ig; Orencia®); Belatacept (L104EA29YIg; LEA29Y; LEA); Ipilimumab (MDX-010; MDX-101); Tremelimumab (ticilimumab; CP-675,206); PRS-010 (see e.g., US20090042785); PRS-050 (U.S. Pat. No. 7,585,940; US20090305982); Aflibercept (VEGF Trap, AVE005; Holash et al., (2002) PNAS 99:11393-11398); Volociximab (M200); F200 (Chimeric (human/murine) IgG4 Fab fragment of Volociximab (M200)); MORAb-009 Mouse/human chimeric IgG1 (US20050054048); Soluble fusion protein:Anti-mesothelin Fv linked to a truncated Pseudomonas exotoxin A (SS1P (CAT-5001); US20070189962); Cixutumumab (IMC-A12); Nimotuzumab (h-R3) (Spicer (2005) Curr Opin Mol Ther 7:182-191); Zalutumumab (HuMax-EGFR; Lammerts van Bueren et al. (2008) PNAS 105:6109-14); Necitumumab IMC-11F8 (Li et al. (2008) Structure 16:216-227); Sym004 (Pedersen et al. 2010 Cancer Res 70:588-597); and mAb-425.

In particular, therapeutic agents include, but are not limited to, immunoglobulins, Interferon beta, Interferon alpha-2as, Interferon alpha-1s, Interferon alpha-n3s, Interferon beta-1, Interferon beta-1 as, Interferon gamma-1bs, Peg-interferon alpha-2 and Peginterferon alpha-2bs, insulin, a bisphosphate (e.g., Pamidronates or Zoledronates), Docetaxels, Doxorubicins, Doxorubicin liposomals and bevacizumabs.

Other exemplary therapeutic agents that can be combined by co-administration and/or co-formulation with a modified PH20 polypeptide provided herein, include, but are not limited to, Adalimumabs, Agalsidase Betas, Alefacepts, Ampicillins, Anakinras, Antipoliomyelitic Vaccines, Anti-Thymocytes, Azithromycins, Becaplermins, Caspofungins, Cefazolins, Cefepimes, Cefotetans, Ceftazidimes, Ceftriaxones, Cetuximabs, Cilastatins, Clavulanic Acids, Clindamycins, Darbepoetin Alfas, Daclizumabs, Diphtheria, Diphtheria antitoxins, Diphtheria Toxoids, Efalizumabs, Epinephrines, Erythropoietin Alphas, Etanercepts, Filgrastims, Fluconazoles, Follicle-Stimulating Hormones, Follitropin Alphas, Follitropin Betas, Fosphenytoins, Gadodiamides, Gadopentetates, Gatifloxacins, Glatiramers, Granulocyte macrophage colony-stimulating factors (GM-CSFs), Goserelins, Goserelin acetates, Granisetrons, Haemophilus Influenza Bs, Haloperidols, Hepatitis vaccines, Hepatitis A Vaccines, Hepatitis B Vaccines, Ibritumomab Tiuxetans, Ibritumomabs, Tiuxetans, Immunoglobulins, Hemophilus influenza vaccines, Influenza Virus Vaccines, Infliximabs, Insulins, Insulin Glargines, Interferons, Interferon alphas, Interferon Betas, Interferon Gammas, Interferon alpha-2as, Interferon alpha-2bs, Interferon alpha-1 s, Interferon alpha-n3s, Interferon Betas, Interferon Beta-1as, Interferon Gammas, Interferon alpha-consensus, Iodixanols, Iohexols, Iopamidols, Ioversols, Ketorolacs, Laronidases, Levofloxacins, Lidocaines, Linezolids, Lorazepams, Measles Vaccines, Measles virus, Mumps viruses, Measles-Mumps-Rubella Virus Vaccines, Rubella vaccines, Medroxyprogesterones, Meropenems, Methylprednisolones, Midazolams, Morphines, Octreotides, Omalizumabs, Ondansetrons, Palivizumabs, Pantoprazoles, Pegaspargases, Pegfilgrastims, Peg-Interferon Alfa-2as, Peg-Interferon Alfa-2bs, Pegvisomants, Pertussis vaccines, Piperacillins, Pneumococcal Vaccines and Pneumococcal Conjugate Vaccines, Promethazines, Reteplases, Somatropins, Sulbactams, Sumatriptans, Tazobactams, Tenecteplases, Tetanus Purified Toxoids, Ticarcillins, Tositumomabs, Triamcinolones, Triamcinolone Acetonides, Triamcinolone hexacetonides, Vancomycins, Varicella Zoster immunoglobulins, Varicella vaccines, other vaccines, Alemtuzumabs, Alitretinoins, Allopurinols, Altretamines, Amifostines, Anastrozoles, Arsenics, Arsenic Trioxides, Asparaginases, Bacillus Calmette-Guerin (BCG) vaccines, BCG Live, Bexarotenes, Bleomycins, Busulfans, Busulfan intravenous, Busulfan orals, Calusterones, Capecitabines, Carboplatins, Carmustines, Carmustines with Polifeprosans, Celecoxibs, Chlorambucils, Cisplatins, Cladribines, Cyclophosphamides, Cytarabines, Cytarabine liposomals, Dacarbazines, Dactinomycins, Daunorubicin liposomals, Daunorubicins, Daunomycins, Denileukin Diftitoxes, Dexrazoxanes, Docetaxels, Doxorubicins, Doxorubicin liposomals, Dromostanolone propionates, Elliotts B Solutions, Epirubicins, Epoetin alfas, Estramustines, Etoposides, Etoposide phosphates, Etoposide VP-16s, Exemestanes, Floxuridines, Fludarabines, Fluorouracils, 5-Fluorouracils, Fulvestrants, Gemcitabines, Gemtuzumabs, Ozogamicins, Gemtuzumab ozogamicins, Hydroxyureas, Idarubicins, Ifosfamides, Imatinib mesylates, Irinotecans, Letrozoles, Leucovorins, Levamisoles, Lomustines, CCNUs, Mechlorethamines, Nitrogen mustards, Megestrols, Megestrol acetates, Melphalans, L-PAMs, Mercaptopurines, 6-Mercaptopurines, Mesnas, Methotrexates, Methoxsalens, Mitomycins, Mitomycin Cs, Mitotanes, Mitoxantrones, Nandrolones, Nandrolone Phenpropionates, Nofetumomabs, Oprelvekins, Oxaliplatins, Paclitaxels, Pamidronates, Pegademases, Pentostatins, Pipobromans, Plicamycins, Mithramycins, Porfimers, Porfimer sodiums, Procarbazines, Quinacrines, Rasburicases, Rituximabs, Sargramostims, Streptozocins, Talcs, Tamoxifens, Temozolomides, Teniposides, Testolactones, Thioguanines, 6-Thioguanines, Triethylenethiophosphoramides (Thiotepas), Topotecans, Toremifenes, Trastuzumabs, Tretinoins, Uracil Mustards, Valrubicins, Vinblastines, Vincristines, Vinorelbines, Zoledronates, Acivicins, Aclarubicins, Acodazoles, Acronines, Adozelesins, Aldesleukins, Retinoic Acids, Alitretinoins, 9-Cis-Retinoic Acids, Alvocidibs, Ambazones, Ambomycins, Ametantrones, Aminoglutethimides, Amsacrines, Anaxirones, Ancitabines, Anthramycins, Apaziquones, Argimesnas, Asperlins, Atrimustines, Azacitidines, Azetepas, Azotomycins, Banoxantrones, Batabulins, Batimastats, Benaxibines, Bendamustines, Benzodepas, Bicalutamides, Bietaserpines, Biricodars, Bisantrenes, Bisnafide Dimesylates, Bizelesins, Bortezomibs, Brequinars, Bropirimines, Budotitanes, Cactinomycins, Canertinibs, Caracemides, Carbetimers, Carboquones, Carmofurs, Carubicins, Carzelesins, Cedefingols, Cemadotins, Chlorambucils, Cioteronels, Cirolemycins, Clanfenurs, Clofarabines, Crisnatols, Decitabines, Dexniguldipines, Dexormaplatins, Dezaguanines, Diaziquones, Dibrospidiums, Dienogests, Dinalins, Disermolides, Dofequidars, Doxifluridines, Droloxifenes, Duazomycins, Ecomustines, Edatrexates, Edotecarins, Eflomithines, Elacridars, Elinafides, Elsamitrucins, Emitefurs, Enloplatins, Enpromates, Enzastaurins, Epipropidines, Eptaloprosts, Erbulozoles, Esorubicins, Etanidazoles, Etoglucids, Etoprines, Exisulinds, Fadrozoles, Fazarabines, Fenretinides, Fluoxymesterones, Flurocitabines, Fosquidones, Fostriecins, Fotretamines, Galarubicins, Galocitabines, Geroquinols, Gimatecans, Gimeracils, Gloxazones, Glufosfamides, Ilmofosines, Ilomastats, Imexons, Improsulfans, Indisulams, Inproquones, Interleukins, Interleukin-2s, recombinant Interleukins, Intoplicines, Iobenguanes, Iproplatins, Irsogladines, Ixabepilones, Ketotrexates, L-Alanosines, Lanreotides, Lapatinibs, Ledoxantrones, Leuprolides, Leuprorelins, Lexacalcitols, Liarozoles, Lobaplatins, Lometrexols, Lonafarnibs, Losoxantrones, Lurtotecans, Mafosfamides, Mannosulfans, Marimastats, Masoprocols, Maytansines, Mechlorethamines, Melengestrols, Melphalans, Menogarils, Mepitiostanes, Metesinds, Metomidates, Metoprines, Meturedepas, Miboplatins, Miproxifenes, Misonidazoles, Mitindomides, Mitocarcins, Mitocromins, Mitoflaxones, Mitogillins, Mitoguazones, Mitomalcins, Mitonafides, Mitoquidones, Mitospers, Mitozolomides, Mivobulins, Mizoribines, Mofarotenes, Mopidamols, Mubritinibs, Mycophenolic Acids, Nedaplatins, Neizarabines, Nemorubicins, Nitracrines, Nocodazoles, Nogalamycins, Nolatrexeds, Nortopixantrones, Ormaplatins, Ortataxels, Oteracils, Oxisurans, Oxophenarsines, Patupilones, Peldesines, Peliomycins, Pelitrexols, Pemetrexeds, Pentamustines, Peplomycins, Perfosfamides, Perifosines, Picoplatins, Pinafides, Piposulfans, Pirfenidones, Piroxantrones, Pixantrones, Plevitrexeds, Plomestanes, Porfiromycins, Prednimustines, Propamidines, Prospidiums, Pumitepas, Puromycins, Pyrazofurins, Ranimustines, Riboprines, Ritrosulfans, Rogletimides, Roquinimexs, Rufocromomycins, Sabarubicins, Safingols, Satraplatins, Sebriplatins, Semustines, Simtrazenes, Sizofirans, Sobuzoxanes, Sorafenibs, Sparfosates, Sparfosic Acids, Sparsomycins, Spirogermaniums, Spiromustines, Spiroplatins, Squalamines, Streptonigrins, Streptovarycins, Sufosfamides, Sulofenurs, Tacedinalines, Talisomycins, Tallimustines, Tariquidars, Tauromustines, Tecogalans, Tegafurs, Teloxantrones, Temoporfins, Teroxirones, Thiamiprines, Tiamiprines, Tiazofurins, Tilomisoles, Tilorones, Timcodars, Timonacics, Tirapazamines, Topixantrones, Trabectedins, Ecteinascidin 743, Trestolones, Triciribines, Trilostanes, Trimetrexates, Triplatin Tetranitrates, Triptorelins, Trofosfamides, Tubulozoles, Ubenimexs, Uredepas, Valspodars, Vapreotides, Verteporfins, Vinblastines, Vindesines, Vinepidines, Vinflunines, Vinformides, Vinglycinates, Vinleucinols, Vinleurosines, Vinrosidines, Vintriptols, Vinzolidines, Vorozoles, Xanthomycin As, Guamecyclines, Zeniplatins, Zilascorbs [2-H], Zinostatins, Zorubicins, Zosuquidars, Acetazolamides, Acyclovirs, Adipiodones, Alatrofloxacins, Alfentanils, Allergenic extracts, Alpha 1-proteinase inhibitors, Alprostadils, Amikacins, Amino acids, Aminocaproic acids, Aminophyllines, Amitriptylines, Amobarbitals, Amrinones, Analgesics, Anti-poliomyelitis vaccines, Anti-rabic serums, Anti-tetanus immunoglobulins, tetanus vaccines, Antithrombin IIIs, Antivenom serums, Argatrobans, Arginines, Ascorbic acids, Atenolols, Atracuriums, Atropines, Aurothioglucoses, Azathioprines, Aztreonams, Bacitracins, Baclofens, Basiliximabs, Benzoic acids, Benztropines, Betamethasones, Biotins, Bivalirudins, Botulism antitoxins, Bretyliums, Bumetanides, Bupivacaines, Buprenorphines, Butorphanols, Calcitonins, Calcitriols, Calciums, Capreomycins, Carboprosts, Carnitines, Cefamandoles, Cefoperazones, Cefotaximes, Cefoxitins, Ceftizoximes, Cefuroximes, Chloramphenicols, Chloroprocaines, Chloroquines, Chlorothiazides, Chlorpromazines, Chondroitinsulfuric acids, Choriogonadotropin alfas, Chromiums, Cidofovirs, Cimetidines, Ciprofloxacins, Cisatracuriums, Clonidines, Codeines, Colchicines, Colistins, Collagens, Corticorelin ovine triflutates, Corticotrophins, Cosyntropins, Cyanocobalamins, Cyclosporines, Cysteines, Dacliximabs, Dalfopristins, Dalteparins, Danaparoids, Dantrolenes, Deferoxamines, Desmopressins, Dexamethasones, Dexmedetomidines, Dexpanthenols, Dextrans, Iron dextrans, Diatrizoic acids, Diazepams, Diazoxides, Dicyclomines, Digibinds, Digoxins, Dihydroergotamines, Diltiazems, Diphenhydramines, Dipyridamoles, Dobutamines, Dopamines, Doxacuriums, Doxaprams, Doxercalciferols, Doxycyclines, Droperidols, Dyphyllines, Edetic acids, Edrophoniums, Enalaprilats, Ephedrines, Epoprostenols, Ergocalciferols, Ergonovines, Ertapenems, Erythromycins, Esmolols, Estradiols, Estrogenics, Ethacrynic acids, Ethanolamines, Ethanols, Ethiodized oils, Etidronic acids, Etomidates, Factor VIIIs, Famotidines, Fenoldopams, Fentanyls, Flumazenils, Fluoresceins, Fluphenazines, Folic acids, Fomepizoles, Fomivirsens, Fondaparinuxs, Foscarnets, Fosphenytoins, Furosemides, Gadoteridols, Gadoversetamides, Ganciclovirs, Gentamicins, Glucagons, Glucoses, Glycines, Glycopyrrolates, Gonadorelins, Gonadotropin chorionics, Haemophilus B polysaccharides, Hemins, Herbals, Histamines, Hydralazines, Hydrocortisones, Hydromorphones, Hydroxocobalamins, Hydroxyzines, Hyoscyamines, Ibutilides, Imiglucerases, Indigo carmines, Indomethacins, Iodides, Iopromides, Iothalamic acids, Ioxaglic acids, Ioxilans, Isoniazids, Isoproterenols, Japanese encephalitis vaccines, Kanamycins, Ketamines, Labetalols, Lepirudins, Levobupivacaines, Levothyroxines, Lincomycins, Liothyronines, Luteinizing hormones, Lyme disease vaccines, Mangafodipirs, Manthtols, Meningococcal polysaccharide vaccines, Meperidines, Mepivacaines, Mesoridazines, Metaraminols, Methadones, Methocarbamols, Methohexitals, Methyldopates, Methylergonovines, Metoclopramides, Metoprolols, Metronidazoles, Minocyclines, Mivacuriums, Morrhuic acids, Moxifloxacins, Muromonab-CD3s, Mycophenolate mofetils, Nafcillins, Nalbuphines, Nalmefenes, Naloxones, Neostigmines, Niacinamides, Nicardipines, Nitroglycerins, Nitroprussides, Norepinephrines, Orphenadrines, Oxacillins, Oxymorphones, Oxytetracyclines, Oxytocins, Pancuroniums, Panthenols, Pantothenic acids, Papaverines, Peginterferon-alpha (e.g., interferon alpha 2a or 2b), Penicillin Gs, Pentamidines, Pentazocines, Pentobarbitals, Perflutrens, Perphenazines, Phenobarbitals, Phentolamines, Phenylephrines, Phenytoins, Physostigmines, Phytonadiones, Polymyxin bs, Pralidoximes, Prilocaines, Procainamides, Procaines, Prochlorperazines, Progesterones, Propranolols, Pyridostigmine hydroxides, Pyridoxines, Quinidines, Quinupristins, Rabies immunoglobulins, Rabies vaccines, Ranitidines, Remifentanils, Riboflavins, Rifampins, Ropivacaines, Samariums, Scopolamines, Seleniums, Sermorelins, Sincalides, Somatrems, Spectinomycins, Streptokinases, Streptomycins, Succinylcholines, Sufentanils, Sulfamethoxazoles, Tacrolimuses, Terbutalines, Teriparatides, Testosterones, Tetanus antitoxins, Tetracaines, Tetradecyl sulfates, Theophyllines, Thiamines, Thiethylperazines, Thiopentals, Thyroid stimulating hormones, Tinzaparins, Tirofibans, Tobramycins, Tolazolines, Tolbutamides, Torsemides, Tranexamic acids, Treprostinils, Trifluoperazines, Trimethobenzamides, Trimethoprims, Tromethamines, Tuberculins, Typhoid vaccines, Urofollitropins, Urokinases, Valproic acids, Vasopressins, Vecuroniums, Verapamils, Voriconazoles, Warfarins, Yellow fever vaccines, Zidovudines, Zincs, Ziprasidone hydrochlorides, Aclacinomycins, Actinomycins, Adriamycins, Azaserines, 6-Azauridines, Carzinophilins, Chromomycins, Denopterins, 6-Diazo-5-Oxo-L-Norleucines, Enocitabines, Floxuridines, Olivomycins, Pirarubicins, Piritrexims, Pteropterins, Tegafurs, Tubercidins, Alteplases, Arcitumomabs, bevacizumabs, Botulinum Toxin Type As, Botulinum Toxin Type Bs, Capromab Pendetides, Daclizumabs, Dornase alfas, Drotrecogin alfas, Imciromab Pentetates, and Iodine-131s.

For example, a modified PH20 polypeptide provided herein can be used in combination therapy methods with an insulin (e.g. fast-acting insulin) to increase subcutaneous delivery of the insulin (see e.g., U.S. Pat. No. 7,767,429; U.S. Pat. No. 7,846,431; U.S. Publication No. US20090304665; and U.S. application Ser. Nos. 13/507,263; 13/507,262 and Ser. No. 13/507,261). Such methods include methods of direct administration, and pump and continuous infusion methods, including open and closed pump systems. For example, exemplary insulins that can be administered with a modified PH20 hyaluronidase provided herein are fast-acting insulins or insulin analogs. For example, a co-administered insulin includes a regular insulin, insulin aspart, insulin lispro, insulin glulisine or other similar analog variants. Exemplary insulins are insulins that contain an A chain set forth in SEQ ID NO:393 and a B chain set forth in SEQ ID NO:394 or variants that contain one or more amino acid modifications compared to a human insulin set forth in SEQ ID NO: 393 and 394 (A and B chains). For example, exemplary insulin analogs are known to one of skill in the art, and include, but are not limited to, those set forth in SEQ ID NOS:393 (A-chain) and having a B-chain set forth in any of SEQ ID NOS: 395-397. The modified PH20 can be co-administered or co-formulated with insulin to treat any condition that is amenable to treatment with insulin. Therapeutic uses include, but are not limited to, treatment for type 1 diabetes mellitus, type 2 diabetes mellitus, gestational diabetes, and for glycemic control in critically ill patients.

2. Methods of Treating Hyaluronan-Associated Diseases and Conditions (e.g., Tumors)

In particular, any of the modified PH20 hyaluronidase can be used to treat hyaluronan-associated diseases or conditions. Typically, hyaluronan-associated diseases and conditions are associated with elevated hyaluronan (HA) expression in a tissue, cell, or body fluid (e.g., tumor tissue or tumor-associated tissue, blood, or interstitial space).

A subject with a hyaluronan-associated disease or condition can be identified to assess if the level of hyaluronan is elevated by comparison of the levels of hyaluronan in a sample (e.g. tissue, cell or body fluid) to a control sample, e.g., another tissue, cell or body fluid. The elevated hyaluronan expression can be elevated compared to a normal tissue, cell or body fluid, for example, a tissue, cell or body fluid that is analogous to the sample being tested, but isolated from a different subject, such as a subject that is normal (i.e., does not have a disease or condition, or does not have the type of disease or condition that the subject being tested has), for example, a subject that does not have a hyaluronan-associated disease or condition. The elevated hyaluronan expression can be elevated compared to an analogous tissue from another subject that has a similar disease or condition, but whose disease is not as severe and/or is not hyaluronan-associated or expresses relatively less hyaluronan and thus is hyaluronan-associated to a lesser degree. For example, the subject being tested can be a subject with a hyaluronan-associated cancer, where the HA amounts in the tissue, cell or fluid are relatively elevated compared to a subject having a less severe cancer, such as an early stage, differentiated or other type of cancer. In another example, the cell, tissue or fluid contains elevated levels of hyaluronan compared to a control sample, such as a fluid, tissue, extract (e.g., cellular or nuclear extract), nucleic acid or peptide preparation, cell line, biopsy, standard or other sample, with a known amount or relative amount of HA, such as a sample, for example a tumor cell line, known to express relatively low levels of HA, such as exemplary tumor cell lines described herein that express low levels of HA, for example, the HCT 116 cell line, the HT29 cell line, the NCI H460 cell line, the DU145 cell line, the Capan-1 cell line, and tumors from tumor models generated using such cell lines.

Hyaluronan-associated diseases and conditions include those associated with high interstitial fluid pressure, such as disc pressure, proliferative disorders, such as cancer and benign prostatic hyperplasia, and edema. Edema can result from or be manifested in, for example, organ transplant, stroke or brain trauma. Proliferative disorders include, but are not limited to, cancer, smooth muscle cell proliferation, systemic sclerosis, cirrhosis of the liver, adult respiratory distress syndrome, idiopathic cardiomyopathy, lupus erythematosus, retinopathy, e.g., diabetic retinopathy or other retinopathies, cardiac hyperplasia, reproductive system associated disorders, such as benign prostatic hyperplasia (BPH) and ovarian cysts, pulmonary fibrosis, endometriosis, fibromatosis, hamartomas, lymphangiomatosis, sarcoidosis, desmoid tumors. Cancers include solid and lymphatic/blood tumors and metastatic disease, and undifferentiated tumors. The tumors amenable to treatment typically exhibit cellular and/or stromal expression of a hyaluronan, compared to a non-cancerous tissue of the same tissue type or compared to a non-metastatic tumor of the same tumor-type. Cancers include any one or more of ovarian cancer, in situ carcinoma (ISC), squamous cell carcinoma (SCC), prostate cancer, pancreatic cancer, other gastric cancers, non-small cell lung cancer, breast cancer, brain cancer and colon cancer.

Modified PH20 polypeptides provided herein, such as PEGylated forms thereof, can be used to treat tumors. Thus, in addition to its indirect anticancer effects, hyaluronidases also have direct anticarcinogenic effects. Hyaluronidase prevents growth of tumors transplanted into mice (De Maeyer et al., 1992, Int. J. Cancer 51:657-660) and inhibits tumor formation upon exposure to carcinogens (Pawlowski et al., 1979, Int. J. Cancer 23:105-109; Haberman et al., 1981, Proceedings of the 17th Annual Meeting of the American Society of Clinical Oncology, Washington, D.C., 22:105, abstract no. 415). PH20 hyaluronidase has been shown to treat various tumors (see e.g., U.S. Publication No. US2010/0003238 and U.S. application Ser. No. 13/135,817, published as U.S. Publication No. US20120020951).

The hyaluronan-rich cancer can be a cancer in which the cancer cells produce HALOs, cancers that have elevated expression of hyaluronan (as determined by immunostaining, e.g., histological staining of sections from the tumor), cancers that have elevated HAS2 (Hyaluronan synthase 2), cancers that do not produce hyaluronidase (HYAL1) in vitro. Hyaluronan-rich cancers can be identified by any method for assessing hyaluronan expression, and other known methods for assaying protein/mRNA expression.

Several hyaluronan-rich cancers have been identified. In some cases, hyaluronan expression correlates with poor prognosis, for example, decreased survival rate and/or recurrence-free survival rate, metastases, angiogenesis, cancer cell invasion into other tissues/areas, and other indicators of poor prognosis. Such correlation has been observed, for example, in hyaluronan-rich tumors including ovarian cancer, SCC, ISC, prostate cancer, lung cancer, including non-small-cell lung cancer (NSCLC), breast cancer, colon cancer and pancreatic cancer (see, for example, Anttila et al., Cancer Research, 60:150-155 (2000); Karvinen et al., British Journal of Dermatology, 148:86-94 (2003); Lipponen et al., Eur. Journal of Cancer, 849-856 (2001); Pirinen et al., Int. J. Cancer: 95: 12-17 (2001); Auvinen et al., American Journal of Pathology, 156(2):529-536 (2000); Ropponen et al., Cancer Research, 58: 342-347 (1998)). Thus, hyaluronan-rich cancers can be treated by administration of a hyaluronidase, such as a modified PH20 hyaluronidase provided herein, to treat one or more symptoms of the cancer. Hyaluronan-rich tumors include, but are not limited to those of the prostate, breast, colon, ovarian, stomach, head and neck and other tumors and cancers.

Other hyaluronan-associated diseases or conditions that are associated with excess glycosaminoglycans and that can be treated with a modified PH20 polypeptide provided herein include, but are not limited to, cardiovascular disease (e.g., following ischemia reperfusion; in arteriosclerosis); vitrectomy and ophthalmic disorders and conditions (e.g., in methods to liquefy the vitreous humor of the eye; reduce postoperative pressure; other ocular surgical procedures such as glaucoma, vitreous and retina surgery and in corneal transplantation); in hypodermoclysis (i.e., infusion of fluids and electrolytes into the hypodermis of the skin); cosmetic applications (e.g., in the treatment of cellulite, “pigskin” edema or “orange peel” edema); organ transplantation (e.g., associated with interstitial edemas in connection with grafting of an organ); and pulmonary disease.

3. Other Uses

In further examples of its therapeutic use, modified PH20 polypeptides provided herein, can be used for such purposes as an antidote to local necrosis from paravenous injection of necrotic substances such as vinca alkaloids (Few et al. (1987) Amer. J. Matern. Child Nurs. 12, 23-26), treatment of ganglion cysts (Paul et al. (1997) J Hand Surg. 22 (2): 219-21) and treatment of tissue necrosis due to venous insufficiency (Elder et al. (1980) Lancet 648-649). Modified PH20 polypeptides also can be used to treat ganglion cysts (also known as a wrist cyst, Bible cyst, or dorsal tendon cyst), which are the most common soft tissue mass of the hand and are fluid filled sacs that can be felt below the skin.

Modified PH20 polypeptides can be used in the treatment of spinal cord injury by degrading chondroitin sulfate proteoglycans (CSPGs). Following spinal cord injury, glial scars containing CSPGs are produced by astrocytes. CSPGs play a crucial role in the inhibition of axon growth. In addition, the expression of CSPG has been shown to increase following injury of the central nervous system (CNS). Soluble PH20 also can be utilized for the treatment of herniated disks in a process known as chemonucleolysis. Chondroitinase ABC, an enzyme cleaving similar substrates as hyaluronidase, can induce the reduction of intradiscal pressure in the lumbar spine. There are three types of disk injuries. A protruded disk is one that is intact but bulging. In an extruded disk, the fibrous wrapper has torn and the NP has oozed out, but is still connected to the disk. In a sequestered disk, a fragment of the NP has broken loose from the disk and is free in the spinal canal. Chemonucleolysis is typically effective on protruded and extruded disks, but not on sequestered disk injuries.

### I. EXAMPLES

The following examples are included for illustrative purposes only and are not intended to limit the scope of the invention.

### Example 1

### Preparation of Recombinant Human PH20 Hyaluronidase (rHuPH20)

A. Generation of a Soluble rHuPH20-Expressing Cell Line

A recombinant human PH20 hyaluronidase designated rHuPH20 was generated as described in published U.S. Publication No. US20110053247, now U.S. Pat. No. 8,187,855. Briefly, Chinese Hamster Ovary (CHO cells) were transfected with the plasmid designated pCI-PH20-IRES-DHFR-SV40pa (HZ24) plasmid, whose sequence is set forth in SEQ ID NO:5 (see e.g., U.S. Pat. Nos. 7,767,429 and 7,781,607 and U.S. Publication No. 2006-0104968). The HZ24 plasmid vector for expression of rHuPH20 contains a pCI vector backbone (Promega), DNA encoding amino acids 1-482 of human PH20 hyaluronidase set forth in SEQ ID NO:6, an internal ribosomal entry site (IRES) from the ECMV virus (Clontech), and the mouse dihydrofolate reductase (DHFR) gene. The vector encodes soluble hyaluronidase and the product is designated rHuPH20. The pCI vector backbone also includes DNA encoding the Beta-lactamase resistance gene (AmpR), an fl origin of replication, a Cytomegalovirus immediate-early enhancer/promoter region (CMV), a chimeric intron, and an SV40 late polyadenylation signal (SV40). The DNA encoding the soluble rHuPH20 construct contains an NheI site and a Kozak consensus sequence prior to the DNA encoding the methionine at amino acid position 1 of the native 35 amino acid signal sequence of human PH20, and a stop codon following the DNA encoding the tyrosine corresponding to amino acid position 482 of the human PH20 hyaluronidase set forth in SEQ ID NO:6, followed by a BamHI restriction site.

Non-transfected DG44 CHO cells growing in GIBCO Modified CD-CHO media for DHFR(−) cells, supplemented with 4 mM Glutamine and 18 mL/L Pluronic F68/L (Gibco), were seeded at 0.5×10⁶ cells/mL in a shaker flask in preparation for transfection. Cells were grown at 37° C. in 5% CO₂ in a humidified incubator, shaking at 120 rpm. Exponentially growing non-transfected DG44 CHO cells were tested for viability prior to transfection.

Sixty million viable cells of the non-transfected DG44 CHO cell culture were pelleted and resuspended to a density of 2×10⁷ cells in 0.7 mL of 2× transfection buffer (2× HeBS: 40 mM HEPES, pH 7.0, 274 mM NaCl, 10 mM KCl, 1.4 mM Na₂HPO₄, 12 mM dextrose). To each aliquot of resuspended cells, 0.09 mL (250 μg) of the linear HZ24 plasmid (linearized by overnight digestion with Cla I (New England Biolabs) was added, and the cell/DNA solutions were transferred into 0.4 cm gap BTX (Gentronics) electroporation cuvettes at room temperature. A negative control electroporation was performed with no plasmid DNA mixed with the cells. The cell/plasmid mixes were electroporated with a capacitor discharge of 330 V and 960 μF or at 350 V and 960 μF.

The cells were removed from the cuvettes after electroporation and transferred into 5 mL of Modified CD-CHO media for DHFR(−) cells, supplemented with 4 mM Glutamine and 18 mL/L Pluronic F68/L (Gibco), and allowed to grow in a well of a 6-well tissue culture plate without selection for 2 days at 37° C. in 5% CO₂ in a humidified incubator.

Two days post-electroporation, 0.5 mL of tissue culture media was removed from each well and tested for the presence of hyaluronidase activity, using the microturbidity assay described in Example 3. The results are set forth in Table 4.

TABLE 4
Initial Hyaluronidase Activity of HZ24 Transfected DG44 CHO
cells at 40 hours post-transfection

| | Dilution | Activity (Units/mL) |
|---|---|---|
| Transfection 1 330 V | 1 to 10 | 0.25 |
| Transfection 2 350 V | 1 to 10 | 0.52 |
| Negative Control | 1 to 10 | 0.015 |

Cells from Transfection 2 (350V) were collected from the tissue culture well, counted and diluted to 1×10⁴ to 2×10⁴ viable cells per mL. A 0.1 mL aliquot of the cell suspension was transferred to each well of five, 96 well round bottom tissue culture plates. One hundred microliters of CD-CHO media (GIBCO) containing 4 mM GlutaMAX™-1 supplement (GIBCO™, Invitrogen Corporation) and without hypoxanthine and thymidine supplements were added to the wells containing cells (final volume 0.2 mL). Ten clones were identified from the 5 plates grown without methotrexate (Table 5).

TABLE 5
Hyaluronidase activity of identified clones

| Plate/Well ID | Relative Hyaluronidase |
|---|---|
| 1C3 | 261 |
| 2C2 | 261 |
| 3D3 | 261 |
| 3E5 | 243 |
| 3C6 | 174 |
| 2G8 | 103 |
| 1B9 | 304 |
| 2D9 | 273 |
| 4D10 | 302 |

Six HZ24 clones were expanded in culture and transferred into shaker flasks as single cell suspensions. Clones 3D3, 3E5, 2G8, 2D9, 1E11, and 4D10 were plated into 96-well round bottom tissue culture plates using a two-dimensional infinite dilution strategy in which cells were diluted 1:2 down the plate, and 1:3 across the plate, starting at 5000 cells in the top left hand well. Diluted clones were grown in a background of 500 non-transfected DG44 CHO cells per well, to provide necessary growth factors for the initial days in culture. Ten plates were made per subclone, with 5 plates containing 50 nM methotrexate and 5 plates without methotrexate.

Clone 3D3 produced 24 visual subclones (13 from the no methotrexate treatment, and 11 from the 50 nM methotrexate treatment). Significant hyaluronidase activity was measured in the supernatants from 8 of the 24 subclones (>50 Units/mL), and these 8 subclones were expanded into T-25 tissue culture flasks. Clones isolated from the methotrexate treatment protocol were expanded in the presence of 50 nM methotrexate. Clone 3D35M was further expanded in 500 nM methotrexate giving rise to clones producing hyaluronidase activity in excess of 1,000 Units/mL in shaker flasks (clone 3D35M; or Gen1 3D35M). A master cell bank (MCB) of the 3D35M cells was then prepared.

B. Production of Gen2 Cells Containing Soluble Human PH20 (rHuPH20)

The Gen1 3D35M cell line described in Example 1.A was adapted to higher methotrexate levels to produce generation 2 (Gen2) clones. 3D35M cells were seeded from established methotrexate-containing cultures into CD CHO medium containing 4 mM GlutaMAX-1™ and 1.0 μM methotrexate. The cells were adapted to a higher methotrexate level by growing and passaging them 9 times over a period of 46 days in a 37° C., 7% CO₂ humidified incubator. The amplified population of cells was cloned out by limiting dilution in 96-well tissue culture plates containing medium with 2.0 μM methotrexate. After approximately 4 weeks, clones were identified and clone 3E10B was selected for expansion. 3E10B cells were grown in CD CHO medium containing 4 mM GlutaMAX-1™ and 2.0 μM methotrexate for 20 passages. A master cell bank (MCB) of the 3E10B cell line was created and frozen and used for subsequent studies.

Amplification of the cell line continued by culturing 3E10B cells in CD CHO medium containing 4 mM GlutaMAX-1™ and 4.0 μM methotrexate. After the 12^{th} passage, cells were frozen in vials as a research cell bank (RCB). One vial of the RCB was thawed and cultured in medium containing 8.0 μM methotrexate. After 5 days, the methotrexate concentration in the medium was increased to 16.0 μM, then 20.0 μM 18 days later. Cells from the 8^{th} passage in medium containing 20.0 μM methotrexate were cloned out by limiting dilution in 96-well tissue culture plates containing CD CHO medium containing 4 mM GlutaMAX-1™ and 20.0 μM methotrexate. Clones were identified 5-6 weeks later and clone 2B2 was selected for expansion in medium containing 20.0 μM methotrexate. After the 11^{th} passage, 2B2 cells were frozen in vials as a research cell bank (RCB).

The resulting 2B2 cells are dihydrofolate reductase deficient (dhfr−) DG44 CHO cells that express soluble recombinant human PH20 (rHuPH20). The soluble PH20 is present in 2B2 cells at a copy number of approximately 206 copies/cell. Southern blot analysis of SpeI-, XbaI- and BamHI/HindIII-digested genomic 2B2 cell DNA using a rHuPH20-specific probe revealed the following restriction digest profile: one major hybridizing band of ˜7.7 kb and four minor hybridizing bands (˜13.9, ˜6.6, ˜5.7 and ˜4.6 kb) with DNA digested with SpeI; one major hybridizing band of ˜5.0 kb and two minor hybridizing bands (˜13.9 and ˜6.5 kb) with DNA digested with XbaI; and one single hybridizing band of ˜1.4 kb observed using 2B2 DNA digested with BamHI/HindIII.

C. Production of Gen2 Soluble rHuPH20 in 300 L Bioreactor Cell Culture

A vial of HZ24-2B2 was thawed and expanded from shaker flasks through 36 L spinner flasks in CD-CHO media (Invitrogen, Carlsbad, Calif.) supplemented with 20 μM methotrexate and GlutaMAX-1™ (Invitrogen). Briefly, the vial of cells was thawed in a 37° C. water bath, medium was added and the cells were centrifuged. The cells were re-suspended in a 125 mL shake flask with 20 mL of fresh medium and placed in a 37° C., 7% CO₂ incubator. The cells were expanded up to 40 mL in the 125 mL shake flask. When the cell density reached greater than 1.5×10⁶ cells/mL, the culture was expanded into a 125 mL spinner flask in a 100 mL culture volume. The flask was incubated at 37° C., 7% CO₂. When the cell density reached greater than 1.5×10⁶ cells/mL, the culture was expanded into a 250 mL spinner flask in 200 mL culture volume, and the flask was incubated at 37° C., 7% CO₂. When the cell density reached greater than 1.5×10⁶ cells/mL, the culture was expanded into a 1 L spinner flask in 800 mL culture volume and incubated at 37° C., 7% CO₂. When the cell density reached greater than 1.5×10⁶ cells/mL the culture was expanded into a 6 L spinner flask in 5000 mL culture volume and incubated at 37° C., 7% CO₂. When the cell density reached greater than 1.5×10⁶ cells/mL the culture was expanded into a 36 L spinner flask in 32 L culture volume and incubated at 37° C., 7% CO₂.

A 400 L reactor was sterilized and 230 mL of CD-CHO media were added. Before use, the reactor was checked for contamination. Approximately 30 L cells were transferred from the 36 L spinner flasks to the 400 L bioreactor (Braun) at an inoculation density of 4.0×10⁵ viable cells per mL and a total volume of 260 L. Parameters were: temperature setpoint, 37° C.; Impeller Speed 40-55 RPM; Vessel Pressure: 3 psi; Air Sparge 0.5-1.5 L/Min.; Air Overlay: 3 L/min. The reactor was sampled daily for cell counts, pH verification, media analysis, protein production and retention. Also, during the run nutrient feeds were added. At 120 hrs (day 5), 10.4 L of Feed #1 Medium (4× CD-CHO+33 g/L Glucose+160 mL/L Glutamax-1™+83 mL/L Yeastolate+33 mg/L rHuInsulin) was added. At 168 hours (day 7), 10.8 L of Feed #2 (2× CD-CHO+33 g/L Glucose+80 mL/L Glutamax-1™+167 mL/L Yeastolate+0.92 g/L Sodium Butyrate) was added, and culture temperature was changed to 36.5° C. At 216 hours (day 9), 10.8 L of Feed #3 (1× CD-CHO+50 g/L Glucose+50 mL/L Glutamax-1™+250 mL/L Yeastolate+1.80 g/L Sodium Butyrate) was added, and culture temperature was changed to 36° C. At 264 hours (day 11), 10.8 L of Feed #4 (1×CD-CHO+33 g/L Glucose+33 mL/L Glutamax-1™+250 mL/L Yeastolate+0.92 g/L Sodium Butyrate) was added, and culture temperature was changed to 35.5° C. The addition of the feed media was observed to dramatically enhance the production of soluble rHuPH20 in the final stages of production. The reactor was harvested at 14 or 15 days or when the viability of the cells dropped below 40%. The process resulted in a final productivity of 17,000 Units per mL with a maximal cell density of 12 million cells/mL. At harvest, the culture was sampled for mycoplasma, bioburden, endotoxin and virus in vitro and in vivo, by Transmission Electron Microscopy (TEM) and enzyme activity.

The culture was pumped by a peristaltic pump through four Millistak filtration system modules (Millipore) in parallel, each containing a layer of diatomaceous earth graded to 4-8 μm and a layer of diatomaceous earth graded to 1.4-1.1 μm, followed by a cellulose membrane, then through a second single Millistak filtration system (Millipore) containing a layer of diatomaceous earth graded to 0.4-0.11 μm and a layer of diatomaceous earth graded to <0.1 μm, followed by a cellulose membrane, and then through a 0.22 μm final filter into a sterile single use flexible bag with a 350 L capacity. The harvested cell culture fluid was supplemented with 10 mM EDTA and 10 mM Tris to a pH of 7.5. The culture was concentrated 10× with a tangential flow filtration (TFF) apparatus using four Sartoslice TFF 30 kDa molecular weight cut-off (MWCO) polyether sulfone (PES) filter (Sartorious), followed by a 10× buffer exchange with 10 mM Tris, 20 mM Na₂SO₄, pH 7.5 into a 0.22 μm final filter into a 50 L sterile storage bag.

The concentrated, diafiltered harvest was inactivated for virus. Prior to viral inactivation, a solution of 10% Triton® X-100, 3% tri (n-butyl) phosphate (TNBP) was prepared. The concentrated, diafiltered harvest was exposed to 1% Triton® X-100, 0.3% TNBP for 1 hour in a 36 L glass reaction vessel immediately prior to purification on the Q column.

D. Purification of Gen2 Soluble rHuPH20

A Q Sepharose (Pharmacia) ion exchange column (9 L resin, H=29 cm, D=20 cm) was prepared. Wash samples were collected for a determination of pH, conductivity and endotoxin (LAL assay). The column was equilibrated with 5 column volumes of 10 mM Tris, 20 mM Na₂SO₄, pH 7.5. Following viral inactivation, the concentrated, diafiltered harvest was loaded onto the Q column at a flow rate of 100 cm/hr. The column was washed with 5 column volumes of 10 mM Tris, 20 mM Na₂SO₄, pH 7.5 and 10 mM HEPES, 50 mM NaCl, pH7.0. The protein was eluted with 10 mM HEPES, 400 mM NaCl, pH 7.0 into a 0.22 μm final filter into sterile bag. The eluate sample was tested for bioburden, protein concentration and hyaluronidase activity. A₂₈₀ absorbance readings were taken at the beginning and end of the exchange.

Phenyl-Sepharose (Pharmacia) hydrophobic interaction chromatography was next performed. A Phenyl-Sepharose (PS) column (19-21 L resin, H=29 cm, D=30 cm) was prepared. The wash was collected and sampled for pH, conductivity and endotoxin (LAL assay). The column was equilibrated with 5 column volumes of 5 mM potassium phosphate, 0.5 M ammonium sulfate, 0.1 mM CaCl₂, pH 7.0. The protein eluate from the Q sepharose column was supplemented with 2M ammonium sulfate, 1 M potassium phosphate and 1 M CaCl₂ stock solutions to yield final concentrations of 5 mM, 0.5 M and 0.1 mM, respectively. The protein was loaded onto the PS column at a flow rate of 100 cm/hr and the column flow thru collected. The column was washed with 5 mM potassium phosphate, 0.5 M ammonium sulfate and 0.1 mM CaCl₂ pH 7.0 at 100 cm/hr and the wash was added to the collected flow thru. Combined with the column wash, the flow through was passed through a 0.22 μm final filter into a sterile bag. The flow through was sampled for bioburden, protein concentration and enzyme activity.

An aminophenyl boronate column (Prometics) was prepared. The wash was collected and sampled for pH, conductivity and endotoxin (LAL assay). The column was equilibrated with 5 column volumes of 5 mM potassium phosphate, 0.5 M ammonium sulfate. The PS flow through containing purified protein was loaded onto the aminophenyl boronate column at a flow rate of 100 cm/hr. The column was washed with 5 mM potassium phosphate, 0.5 M ammonium sulfate, pH 7.0. The column was washed with 20 mM bicine, 0.5 M ammonium sulfate, pH 9.0. The column was washed with 20 mM bicine, 100 mM sodium chloride, pH 9.0. The protein was eluted with 50 mM HEPES, 100 mM NaCl, pH 6.9 and passed through a sterile filter into a sterile bag. The eluted sample was tested for bioburden, protein concentration and enzyme activity.

The hydroxyapatite (HAP) column (Biorad) was prepared. The wash was collected and tested for pH, conductivity and endotoxin (LAL assay). The column was equilibrated with 5 mM potassium phosphate, 100 mM NaCl, 0.1 mM CaCl₂, pH 7.0. The aminophenyl boronate purified protein was supplemented to final concentrations of 5 mM potassium phosphate and 0.1 mM CaCl₂ and loaded onto the HAP column at a flow rate of 100 cm/hr. The column was washed with 5 mM potassium phosphate, pH 7, 100 mM NaCl, 0.1 mM CaCl₂. The column was next washed with 10 mM potassium phosphate, pH 7, 100 mM NaCl, 0.1 mM CaCl₂. The protein was eluted with 70 mM potassium phosphate, pH 7.0 and passed through a 0.22 μm sterile filter into a sterile bag. The eluted sample was tested for bioburden, protein concentration and enzyme activity.

The HAP purified protein was then passed through a virus removal filter. The sterilized Viosart filter (Sartorius) was first prepared by washing with 2 L of 70 mM potassium phosphate, pH 7.0. Before use, the filtered buffer was sampled for pH and conductivity. The HAP purified protein was pumped via a peristaltic pump through the 20 nM virus removal filter. The filtered protein in 70 mM potassium phosphate, pH 7.0 was passed through a 0.22 μm final filter into a sterile bag. The filtered sample was tested for protein concentration, enzyme activity, oligosaccharide, monosaccharide and sialic acid profiling. The sample also was tested for process related impurities.

The protein in the filtrate was then concentrated to 10 mg/mL using a 10 kDa molecular weight cut off (MWCO) Sartocon Slice tangential flow filtration (TFF) system (Sartorius). The filter was first prepared by washing with 10 mM histidine, 130 mM NaCl, pH 6.0 and the permeate was sampled for pH and conductivity. Following concentration, the concentrated protein was sampled and tested for protein concentration and enzyme activity. A 6× buffer exchange was performed on the concentrated protein into the final buffer: 10 mM histidine, 130 mM NaCl, pH 6.0. Following buffer exchange, the concentrated protein was passed though a 0.22 μm filter into a 20 L sterile storage bag. The protein was sampled and tested for protein concentration, enzyme activity, free sulfydryl groups, oligosaccharide profiling and osmolality. Lot number WRS2 was used as a standard in the assays described below, the results showed that the test description for appearance was clear and colorless; the pH was 7.4; the endotoxin level was <0.01 EU/mL; the osmolality was 308 mOsm/Kg; the density was 1.005 g/mL; the rHuPH20 content was 1.3 ppm; and the hyaluronidase activity was 145 USP U/mL.

The sterile filtered bulk protein was then asceptically dispensed at 20 mL into 30 mL sterile Teflon vials (Nalgene). The vials were then flash frozen and stored at −20±5° C.

### Example 2

### Generation of a PH20 Mutant Library

### A. Cloning and Mutagenesis

In this example, a human hyaluronidase PH20 library was created by cloning DNA encoding human PH20 into a plasmid followed by transfection and protein expression.

The library was created by mutagenesis of a PH20 template that is a codon optimized version of PH20 with an Ig Kappa leader sequence. Specifically, for generating the library of variants, the HZ24-PH20(OHO)-IRES-SEAP expression vector (set forth in SEQ ID NO:4) was used as a template, which contains the sequence of nucleotides encoding PH20 set forth in SEQ ID NO:1, which encodes a PH20 set forth in SEQ ID NO:2 or a mature PH20 set forth in SEQ ID NO:3 lacking residues 1-22 corresponding to the IgK signal sequence. The backbone of the vector was derived from the original HZ24 vector containing the DHFR selection marker (see Example 1 and SEQ ID NO:5) with the addition of an IgK leader sequence and codon optimization. The expression vector also was modified to contain the gene for secreted alkaline phosphatase (SEAP). Hence, in addition to sequence encoding PH20, the HZ24-PH20(OHO)-IRES-SEAP expression vector also contains an internal ribosome entry site (EMCV IRES) that is linked to the coding sequence for the gene for secreted alkaline phosphatase (SEAP), and a single CMV promoter that drives expression of PH20 and SEAP in the construct. It also contains a gene for ampilcillin resistance. With reference to the sequence of nucleotides set forth in SEQ ID NO:4, the sequence of nucleotides encoding PH20 corresponds to nucleotides 1058-2464 (including the IgK leader sequence), the sequence of nucleotides encoding SEAP corresponds to nucleotides 2970-4529, and the ampicillin resistance gene corresponds to nucleotides 5778-6635.

The first library was made to generate encoded variant proteins wherein each of residues 23-469 of SEQ ID NO:2 (corresponding to residues 1-447 of SEQ ID NO:3 or residues 36-482 of SEQ ID NO:6) was changed to one of about 15 amino acid residues, such that each member contained a single amino change. The resulting library contained 6753 variant members, each containing a single amino acid mutation compared to residues 23-469 of SEQ ID NO:2 (corresponding to residues 1-447 of SEQ ID NO:3 or residues 36-482 of SEQ ID NO:6). Glycerol stocks of the resulting library were prepared and stored at −80° C. The amino acid replacements (mut) in each member are listed in Table 6 below, and correspond to amino acid replacements with reference to the sequence of amino acids of PH20 set forth in SEQ ID NO:3 (and SEQ ID NOS: 7 or 32-66, which are the mature sequence of PH20 or other C-terminally truncated fragments thereof). The corresponding mutated codons (cod) of each PH20 variant in the library are also listed in Table 6, and correspond to nucleotide residue changes in the corresponding encoding nucleotide for PH20 set forth as 1058-2464 of SEQ ID NO:4. Each member was expressed and screened for hyaluronidase activity as described below.

TABLE 6
PH20 Variants

| mut | cod | mut | cod | mut | cod | mut | cod | mut | cod | mut | cod |
|---|---|---|---|---|---|---|---|---|---|---|---|
| L001A | GCG | Y066S | AGT | R132N | AAT | G198T | ACT | V265G | GGT | I331K | AAG |
| L001C | TGT | Y066T | ACG | R132P | CCT | G198V | GTT | V265H | CAT | I331L | CTG |
| L001D | GAT | Y066V | GTG | R132Q | CAG | G198W | TGG | V265I | ATT | I331Q | CAG |
| L001E | GAG | I067C | TGT | R132S | AGT | G198Y | TAT | V265K | AAG | I331R | CGT |
| L001F | TTT | I067D | GAT | R132T | ACT | Y199A | GCG | V265L | CTG | I331S | AGT |
| L001G | GGT | I067E | GAG | R132V | GTG | Y199C | TGT | V265M | ATG | I331T | ACT |
| L001H | CAT | I067F | TTT | R132Y | TAT | Y199E | GAG | V265N | AAT | I331W | TGG |
| L001K | AAG | I067G | GGG | S133A | GCT | Y199G | GGG | V265P | CCT | I331Y | TAT |
| L001N | AAT | I067H | CAT | S133D | GAT | Y199H | CAT | V265Q | CAG | I332A | GCT |
| L001P | CCG | I067L | TTG | S133E | GAG | Y199I | ATT | V265R | AGG | I332C | TGT |
| L001Q | CAG | I067N | AAT | S133F | TTT | Y199K | AAG | V265S | TCT | I332D | GAT |
| L001R | CGG | I067P | CCG | S133G | GGG | Y199L | CTT | V265W | TGG | I332E | GAG |
| L001S | TCT | I067Q | CAG | S133H | CAT | Y199N | AAT | V265Y | TAT | I332F | TTT |
| L001T | ACG | I067R | CGG | S133I | ATT | Y199P | CCT | F266A | GCG | I332G | GGT |
| L001V | GTG | I067T | ACG | S133L | CTG | Y199Q | CAG | F266C | TGT | I332H | CAT |
| L001W | TGG | I067V | GTT | S133M | ATG | Y199R | AGG | F266D | GAT | I332K | AAG |
| N002A | GCT | I067W | TGG | S133N | AAT | Y199S | TCG | F266G | GGG | I332L | CTG |
| N002C | TGT | I067Y | TAT | S133P | CCT | Y199T | ACG | F266H | CAT | I332N | AAT |
| N002F | TTT | D068A | GCT | S133R | CGG | Y199W | TGG | F266L | CTT | I332P | CCT |
| N002G | GGG | D068C | TGT | S133T | ACT | N200A | GCT | F266M | CCG | I332R | AGG |
| N002H | CAT | D068E | GAG | S133V | GTT | N200D | GAT | F266P | ATG | I332S | AGT |
| N002I | ATT | D068G | GGG | S133W | TGG | N200F | CAG | F266Q | CAG | I332T | ACT |
| N002K | AAG | D068H | CAC | I134A | GCT | N200G | GGT | F266R | CGG | I332Y | TAT |
| N002L | TTG | D068I | ATT | I134C | TGT | N200H | CAT | F266S | TCG | N333A | GCT |
| N002P | CCG | D068K | AAG | I134D | GAT | N200K | AAG | F266T | ACG | N333E | GAG |
| N002Q | CAG | D068L | TTG | I134F | TTT | N200L | CTG | F266V | GTG | N333G | GGT |
| N002S | AGT | D068P | CCT | I134G | GGG | N200M | ATG | F266W | TGG | N333H | CAT |
| N002T | ACG | D068Q | CAG | I134H | CAT | N200P | CCT | F266Y | TAT | N333I | ATT |
| N002V | GTT | D068R | CGG | I134K | AAG | N200Q | CAG | A267D | GAT | N333K | AAG |
| N002W | TGG | D068S | TCG | I134L | TTG | N200R | AGG | A267E | GAG | N333L | CTG |
| N002Y | TAT | D068T | ACT | I134P | CCT | N200S | TCT | A267G | GGT | N333M | ATG |
| F003A | GCT | D068V | GTG | I134Q | CAG | N200T | ACT | A267H | CAT | N333P | CCT |
| F003E | GAG | D068Y | TAT | I134R | CGT | N200V | GTG | A267I | ATT | N333R | CGG |
| F003G | GGG | S069A | GCT | I134S | TCG | N200W | TGG | A267K | AAG | N333S | AGT |
| F003H | CAT | S069C | TGT | I134T | ACT | N200Y | TAT | A267L | CTT | N333T | ACT |
| F003I | ATT | S069E | GAG | I134V | GTG | G201A | GCG | A267M | ATG | N333V | GTT |
| F003K | AAG | S069F | TTT | I134W | TGG | G201E | GAG | A267N | AAT | N333W | TGG |
| F003L | TTG | S069G | GGG | E135A | GCT | G201F | TTT | A267P | CCG | N333Y | TAT |
| F003M | ATG | S069I | ATT | E135C | TGT | G201H | CAT | A267R | AGG | V334A | GCT |
| F003N | AAT | S069L | CTT | E135D | GAT | G201K | AAG | A267S | TCT | V334C | TGT |
| F003P | CCT | S069M | ATG | E135F | TTT | G201L | CTT | A267T | GTG | V334D | GAT |
| F003R | CGT | S069N | AAT | E135G | GGG | G201M | ATG | A267V | ACT | V334E | GAG |
| F003S | TCG | S069P | CCT | E135H | CAT | G201N | AAT | A267W | TGG | V334G | GGG |
| F003T | ACT | S069R | CGT | E135K | AAG | G201P | CCT | Y268A | GCT | V334H | CAT |
| F003V | GTG | S069T | ACG | E135L | TTG | G201Q | CAG | Y268C | TGT | V334L | TTG |
| F003Y | TAT | S069V | GTT | E135N | AAT | G201R | CGT | Y268F | TTT | V334M | ATG |
| R004A | GCG | S069W | TGG | E135P | CCT | G201S | TCG | Y268G | GGG | V334N | AAT |
| R004D | GAT | S069Y | TAT | E135Q | CAG | G201T | ACG | Y268H | CAT | V334P | CCT |
| R004E | GAG | I070A | GCT | E135R | CGG | G201V | GTG | Y268K | AAG | V334Q | CAG |
| R004F | TTT | I070C | TGT | E135S | TCT | G201W | TGG | Y268L | CTT | V334R | AGG |
| R004G | GGG | I070F | TTT | E135W | TGG | S202A | GCG | Y268N | AAT | V334S | TCT |
| R004I | ATT | I070G | GGG | E135Y | TAT | S202E | GAG | Y268P | CCT | V334T | ACT |
| R004L | TTG | I070H | CAT | L136A | GCT | S202F | TTT | Y268Q | CAG | V334Y | TAT |
| R004M | ATG | I070K | AAG | L136C | TGT | S202G | GGT | Y268R | CGT | T335A | GCT |
| R004N | AAT | I070L | TTG | L136D | GAT | S202H | CAT | Y268S | TCG | T335C | TGT |
| R004P | CCT | I070N | AAT | L136F | TTT | S202K | AAG | Y268T | ACT | T335F | TTT |
| R004S | TCT | I070P | CCG | L136G | GGT | S202M | ATG | Y268V | GTG | T335G | GGT |
| R004T | ACG | I070Q | CAG | L136H | CAT | S202N | AAT | Y268W | TGG | T335H | CAT |
| R004V | GTG | I070R | CGT | L136I | ATT | S202P | CCT | T269A | GCT | T335I | ATT |
| R004W | TGG | I070S | TCT | L136M | ATG | S202Q | CAG | T269C | TGT | T335K | AAG |
| R004Y | TAT | I070T | ACT | L136N | AAT | S202R | CGT | T269D | GAT | T335L | TTG |
| A005D | GAT | I070V | GTT | L136P | CCT | S202T | ACG | T269E | GAG | T335N | AAT |
| A005G | GGG | I070Y | TAT | L136Q | CAG | S202V | GTT | T269G | GGT | T335P | CCT |
| A005H | CAT | T071A | GCT | L136R | CGT | S202W | TGG | T269K | AAG | T335Q | CAG |
| A005I | ATT | T071C | TGT | L136S | TCG | S202Y | TAT | T269L | CTG | T335S | TCT |
| A005L | CTT | T071D | GAT | L136T | ACT | C203A | GCG | T269M | ATG | T335V | GTG |
| A005M | ATG | T071E | GAG | L136W | TGG | C203D | GAT | T269N | AAT | T335W | TGG |
| A005N | AAT | T071G | GGG | V137A | GCT | C203E | GAG | T269P | CCG | T335Y | TAT |
| A005P | CCG | T071H | CAT | V137C | TGT | C203G | GGG | T269Q | CAG | L336A | GCT |
| A005Q | CAG | T071L | TTG | V137E | GAG | C203H | CAT | T269R | AGG | L336E | GAG |
| A005R | AGG | T071M | ATG | V137F | TTT | C203L | CTT | T269S | TCG | L336F | TTT |
| A005S | TCG | T071N | AAT | V137G | GGG | C203M | ATG | T269V | GTG | L336G | GGG |
| A005T | ACG | T071P | CCT | V137H | CAT | C203N | AAT | T269Y | TAT | L336H | CAT |
| A005V | GTG | T071Q | CAG | V137I | ATT | C203P | CCG | R270A | GCT | L336K | AAG |
| A005W | TGG | T071R | CGG | V137L | TTG | C203Q | CAG | R270C | TGT | L336M | ATG |
| A005Y | TAT | T071S | TCG | V137N | AAT | C203R | AGG | R270D | GAT | L336N | AAT |
| P006A | GCG | T071V | GTG | V137P | CCT | C203S | AGT | R270E | GAG | L336P | CCT |
| P006D | GAT | T071Y | TAT | V137Q | CAG | C203T | ACT | R270F | TTT | L336R | AGG |
| P006E | GAG | G072A | GCT | V137R | CGT | C203V | GTG | R270G | GGG | L336S | TCT |
| P006F | TTT | G072C | TGT | V137S | TCT | C203W | TGG | R270H | CAT | L336T | ACT |
| P006G | GGG | G072D | GAT | V137T | ACT | F204A | GCG | R270I | ATT | L336V | GTG |
| P006H | CAT | G072E | GAG | V137W | TGG | F204C | TGT | R270M | ATG | L336W | TGG |
| P006K | AAG | G072F | TTT | V137Y | TAT | F204E | GAG | R270N | AAT | L336Y | TAT |
| P006L | CTT | G072H | CAT | Q138A | GCT | F204G | GGG | R270P | CCT | A337C | TGT |
| P006N | AAT | G072I | ATT | Q138C | TGT | F204H | CAT | R270Q | CAG | A337F | TTT |
| P006Q | CAG | G072K | AAG | Q138E | GAG | F204I | ATT | R270S | TCG | A337G | GGG |
| P006R | AGG | G072L | TTG | Q138F | TTT | F204K | AAG | R270T | ACT | A337H | CAT |
| P006S | AGT | G072M | ATG | Q138G | GGG | F204L | CTT | R270V | GTG | A337I | ATT |
| P006T | ACG | G072P | CCT | Q138H | CAT | F204M | ATG | R270Y | TAT | A337K | AAG |
| P006V | GTG | G072Q | CAG | Q138I | ATT | F204P | CCT | I271A | GCT | A337L | TTG |
| P006W | TGG | G072R | CGG | Q138L | TTG | F204Q | CAG | I271D | GAT | A337M | ATG |
| P006Y | TAT | G072S | TCT | Q138M | ATG | F204R | AGG | I271E | GAG | A337N | AAT |
| P007A | GCT | G072T | ACT | Q138N | AAT | F204S | AGT | I271F | TTT | A337P | CCT |
| P007C | TGT | G072V | GTG | Q138R | CGT | F204T | ACT | I271G | GGG | A337R | CGG |
| P007D | GAT | G072W | TGG | Q138S | AGT | F204V | GTG | I271H | CAT | A337S | TCT |
| P007F | TTT | G072Y | TAT | Q138V | GTT | F204W | TGG | I271K | AAG | A337T | ACT |
| P007G | GGT | V073A | GCG | Q138W | TGG | N205A | GCG | I271L | CTT | A337V | GTT |
| P007H | CAT | V073C | TGT | Q138Y | TAT | N205D | GAT | I271M | ATG | A337W | TGG |
| P007I | ATT | V073D | GAT | Q139A | GCT | N205E | GAG | I271P | CCT | A338C | TGT |
| P007K | AAG | V073F | TTT | Q139C | TGT | N205F | TTT | I271R | AGG | A338D | GAT |
| P007L | TTG | V073G | GGG | Q139D | GAT | N205G | GGG | I271S | AGT | A338E | GAG |
| P007M | ATG | V073H | CAT | Q139E | GAG | N205K | AAG | I271T | ACT | A338F | TTT |
| P007Q | CAG | V073K | AAG | Q139F | TTT | N205L | CTG | I271V | GTT | A338G | GGG |
| P007R | CGG | V073L | CTT | Q139G | GGG | N205M | ATG | I271W | TGG | A338H | CAT |
| P007S | AGT | V073M | ATG | Q139H | CAT | N205P | CCT | V272A | GCT | A338I | ATT |
| P007T | ACT | V073P | CCG | Q139K | AAG | N205R | AGG | V272C | TGT | A338K | AAG |
| P007V | GTG | V073Q | CAG | Q139L | CTG | N205S | TCG | V272D | GAT | A338L | CTT |
| P007W | TGG | V073R | TGG | Q139M | ATG | N205T | ACG | V272E | GAG | A338P | CCT |
| P007Y | TAT | V073S | TCG | Q139P | CCT | N205V | GTG | V272G | GGG | A338Q | CAG |
| V008A | GCT | V073T | ACG | Q139R | CGT | N205W | TGG | V272H | CAT | A338R | CGT |
| V008D | GAT | V073W | CGG | Q139S | TCT | N205Y | TAT | V272K | AAG | A338S | TCG |
| V008E | GAG | T074A | GCT | Q139T | ACT | V206C | TGT | V272L | TTG | A338T | ACT |
| V008G | GGT | T074C | TGT | Q139V | GTG | V206D | GAT | V272M | ATG | A338V | GTG |
| V008H | CAT | T074E | GAG | Q140A | GCT | V206F | TTT | V272N | AAT | K339D | GAT |
| V008I | ATT | T074F | TTT | Q140C | TGT | V206G | GGG | V272P | CCT | K339E | GAG |
| V008L | TTG | T074G | GGT | Q140D | GAT | V206H | CAT | V272R | AGG | K339F | TTT |
| V008M | ATG | T074H | CAT | Q140F | TTT | V206I | ATT | V272S | TCG | K339G | GGG |
| V008N | AAT | T074K | AAG | Q140G | GGG | V206K | AAG | V272T | ACT | K339H | CAT |
| V008P | CCT | T074L | TTG | Q140H | CAT | V206L | CTT | V272W | TGG | K339L | CTG |
| V008Q | CAG | T074M | ATG | Q140I | ATT | V206M | ATG | F273A | GCT | K339M | ATG |
| V008R | CGG | T074N | AAT | Q140K | AAG | V206P | CCG | F273C | TGT | K339N | AAT |
| V008S | TCT | T074P | CCG | Q140L | TTG | V206Q | CAG | F273D | GAT | K339P | CCT |
| V008T | ACT | T074R | CGG | Q140M | ATG | V206R | CGG | F273G | GGG | K339R | CGG |
| V008W | TGG | T074S | TCG | Q140R | CGG | V206S | TCT | F273H | CAT | K339S | AGT |
| I009A | GCT | T074V | GTG | Q140S | AGT | V206T | ACG | F273I | ATT | K339T | ACT |
| I009C | TGT | T074W | TGG | Q140V | GTG | V206Y | TAT | F273L | CTG | K339V | GTT |
| I009D | GAT | V075A | GCG | Q140W | TGG | E207A | GCT | F273P | CCT | K339W | TGG |
| I009E | GAG | V075C | TGT | Q140Y | TAT | E207F | TTT | F273Q | CAG | K339Y | TAT |
| I009G | GGG | V075D | GAT | N141A | GCT | E207G | GGG | F273R | CGG | M340A | GCT |
| I009H | CAT | V075F | TTT | N141D | GAT | E207H | CAT | F273S | TCG | M340C | TGT |
| I009K | AAG | V075G | GGG | N141E | GAG | E207I | ATT | F273T | ACG | M340D | GAT |
| I009L | CTT | V075H | CAT | N141F | TTT | E207K | AAG | F273V | GTT | M340E | GAG |
| I009N | AAT | V075L | CTT | N141G | GGT | E207L | TTG | F273W | TGG | M340F | TTT |
| I009P | CCT | V075M | ATG | N141H | CAT | E207M | ATG | F273Y | TAT | M340G | GGG |
| I009Q | CAG | V075N | AAT | N141L | TTG | E207P | CCG | T274A | GCG | M340H | CAT |
| I009R | CGG | V075P | CCG | N141M | ATG | E207Q | CAG | T274C | TGT | M340K | AAG |
| I009S | AGT | V075Q | CAG | N141P | CCT | E207R | AGG | T274E | GAG | M340L | CTG |
| I009T | ACG | V075R | CGT | N141Q | CAG | E207S | TCT | T274F | ATG | M340P | CCT |
| I009V | GTT | V075S | TCT | N141R | CGT | E207T | ACG | T274G | GGG | M340R | CGG |
| P010D | GAT | V075T | ACT | N141S | TCT | E207V | GTT | T274H | CAT | M340S | TCG |
| P010E | GAG | V075W | TGG | N141T | ACT | E207W | TGG | T274L | CTG | M340T | ACT |
| P010F | TTT | V075Y | TAT | N141V | GTT | I208A | GCT | T274N | AAT | M340V | GTG |
| P010G | GGT | N076A | GCT | N141W | TGG | I208C | TGT | T274P | CCT | M340W | TGG |
| P010H | CAT | N076C | TGT | N141Y | TAT | I208D | GAT | T274Q | CAG | C341A | GCT |
| P010I | ATT | N076D | GAT | V142C | TGT | I208E | GAG | T274R | CGT | C341E | GAG |
| P010L | CTT | N076F | TTT | V142D | GAT | I208G | GGG | T274S | AGT | C341G | GGG |
| P010M | ATG | N076G | GGG | V142E | GAG | I208K | AAG | T274V | GTT | C341H | CAT |
| P010N | AAT | N076I | ATT | V142G | GGG | I208L | TTG | T274W | TGG | C341K | AAG |
| P010Q | CAG | N076K | AAG | V142H | CAT | I208M | ATG | T274Y | TAT | C341L | TTG |
| P010R | CGG | N076L | CTG | V142I | ATT | I208P | CCG | D275A | GCT | C341M | ATG |
| P010S | TCG | N076P | CCT | V142K | AAG | I208Q | CAG | D275C | TGT | C341N | AAT |
| P010T | ACT | N076Q | CAG | V142L | TTG | I208R | CGT | D275E | GAG | C341Q | CAG |
| P010W | TGG | N076R | CGT | V142M | ATG | I208S | AGT | D275F | TTT | C341R | AGG |
| P010Y | TAT | N076S | AGT | V142N | AAT | I208T | ACG | D275G | GGG | C341S | TCT |
| N011A | GCG | N076T | ACT | V142P | CCT | I208V | GTG | D275I | ATT | C341T | ACT |
| N011C | TGT | N076V | GTT | V142Q | CAG | I208W | TGG | D275K | AAG | C341V | GTT |
| N011D | GAT | N076W | TGG | V142R | CGG | K209A | GCG | D275L | CTT | C341W | TGG |
| N011E | GAG | G077D | GAT | V142S | AGT | K209C | TGT | D275M | ATG | C341Y | TAT |
| N011F | TTT | G077E | GAG | V142T | ACT | K209D | GAT | D275Q | CAG | S342A | GCT |
| N011G | GGG | G077F | TTT | Q143C | TGT | K209E | GAG | D275R | CGT | S342D | GAT |
| N011H | CAT | G077H | CAT | Q143E | GAG | K209F | TTT | D275S | TCG | S342E | GAG |
| N011I | ATT | G077K | AAG | Q143F | TTT | K209G | GGT | D275T | ACT | S342F | TTT |
| N011K | AAG | G077L | TTG | Q143G | GGG | K209L | CTG | D275V | GTG | S342G | GGG |
| N011L | CTG | G077M | ATG | Q143H | CAT | K209N | AAT | D275W | TGG | S342H | CAT |
| N011P | CCG | G077N | AAT | Q143I | ATT | K209P | CCG | Q276C | TGT | S342I | ATT |
| N011S | TCG | G077P | CCG | Q143K | AAG | K209R | CGG | Q276D | GAT | S342K | AAG |
| N011T | ACG | G077Q | CAG | Q143L | TTG | K209S | AGT | Q276E | GAG | S342L | TTG |
| N011W | TGG | G077R | CGT | Q143M | ATG | K209T | ACT | Q276F | TTT | S342M | ATG |
| N011Y | TAT | G077S | TCG | Q143N | AAT | K209V | GTT | Q276G | GGG | S342P | CCT |
| V012A | GCT | G077T | ACG | Q143P | CCT | K209W | TGG | Q276H | CAT | S342Q | CAG |
| V012D | GAT | G077V | GTG | Q143R | CGG | K209Y | TAT | Q276I | ATT | S342R | CGG |
| V012E | GAG | G077Y | TAT | Q143S | TCG | R210A | GCG | Q276L | CTT | S342T | ACT |
| V012G | GGG | G078A | GCG | Q143T | ACT | R210C | TGT | Q276M | ATG | S342Y | TAT |
| V012H | CAT | G078C | TGT | Q143V | GTG | R210D | GAT | Q276P | CCT | Q343C | TGT |
| V012I | ATT | G078D | GAT | Q143Y | TAT | R210E | GAG | Q276R | CGT | Q343D | GAT |
| V012K | AAG | G078H | CAT | L144A | GCT | R210G | GGT | Q276S | AGT | Q343E | GAG |
| V012L | CTT | G078I | ATT | L144E | GAG | R210K | AAG | Q276V | GTT | Q343F | TTT |
| V012M | ATG | G078K | AAG | L144F | TTT | R210L | CTG | Q276W | TGG | Q343G | GGG |
| V012N | AAT | G078L | TTG | L144G | GGG | R210M | ATG | Q276Y | TAT | Q343I | ATT |
| V012P | CCG | G078M | ATG | L144I | ATT | R210N | AAT | V277A | GCT | Q343L | CTT |
| V012R | AGG | G078P | CCG | L144K | AAG | R210P | CCT | V277C | TGT | Q343M | ATG |
| V012S | TCG | G078Q | CAG | L144N | AAT | R210S | TCG | V277D | GAT | Q343P | CCT |
| V012T | ACT | G078R | AGG | L144P | CCT | R210T | ACT | V277E | GAG | Q343R | AGG |
| V012W | TGG | G078S | TCG | L144Q | CAG | R210V | GTG | V277G | GGG | Q343S | AGT |
| P013A | GCT | G078T | ACT | L144R | CGT | R210W | TGG | V277H | CAT | Q343T | ACT |
| P013E | GAG | G078V | GTG | L144S | TCT | R210Y | TAT | V277K | AAG | Q343V | GTG |
| P013F | TTT | G078Y | TAT | L144T | ACT | N211A | GCG | V277L | TTG | Q343W | TGG |
| P013G | GGG | I079A | GCT | L144V | GTT | N211C | TGT | V277M | ATG | Q343Y | TAT |
| P013H | CAT | I079D | GAT | L144W | TGG | N211F | TTT | V277N | AAT | V344E | GAG |
| P013I | ATT | I079F | TTT | L144Y | TAT | N211G | GGG | V277Q | CAG | V344F | TTT |
| P013L | CTT | I079G | GGG | S145A | GCT | N211H | CAT | V277R | AGG | V344G | GGG |
| P013M | ATG | I079H | CAT | S145C | TGT | N211I | ATT | V277S | TCT | V344H | CAT |
| P013Q | CAG | I079K | AAG | S145D | GAT | N211K | AAG | V277T | ACT | V344I | ATT |
| P013R | CGT | I079L | TTG | S145E | GAG | N211L | CTG | V277Y | TAT | V344L | CTG |
| P013S | TCG | I079N | AAT | S145F | TTT | N211M | ATG | L278A | GCT | V344M | ATG |
| P013T | ACT | I079P | CCG | S145G | GGG | N211P | CCT | L278E | GAG | V344N | AAT |
| P013V | GTG | I079R | CGT | S145H | CAT | N211R | CGG | L278F | TTT | V344P | CCT |
| P013W | TGG | I079S | AGT | S145L | TTG | N211S | AGT | L278G | GGG | V344Q | CAG |
| P013Y | TAT | I079T | ACT | S145M | ATG | N211T | ACT | L278H | CAT | V344R | CGT |
| F014A | GCG | I079V | GTT | S145N | AAT | N211V | GTT | L278I | ATT | V344S | TCG |
| F014D | GAT | I079W | TGG | S145P | CCT | N211W | TGG | L278K | AAG | V344T | ACT |
| F014E | GAG | I079Y | TAT | S145R | CGT | D212A | GCT | L278M | TTT | V344W | TGG |
| F014G | GGT | P080A | GCG | S145T | ACT | D212E | GAG | L278N | AAT | V344Y | TAT |
| F014H | CAT | P080D | GAT | S145V | GTT | D212G | GGG | L278P | CCG | L345A | GCT |
| F014I | ATT | P080E | GAG | S145W | TGG | D212H | CAT | L278R | CGT | L345C | TGT |
| F014K | AAG | P080F | TTT | L146A | GCT | D212I | ATT | L278S | TCT | L345D | GAT |
| F014M | ATG | P080G | GGG | L146C | TGT | D212K | AAG | L278T | ACT | L345E | GAG |
| F014N | AAT | P080I | ATT | L146E | GAG | D212L | CTG | L278V | GTT | L345G | GGG |
| F014P | CCT | P080K | AAG | L146G | GGG | D212M | ATG | L278Y | TAT | L345H | CAT |
| F014Q | CAG | P080L | CTT | L146H | CAT | D212N | AAT | K279A | GCG | L345K | AAG |
| F014R | CGG | P080M | ATG | L146I | ATT | D212P | CCT | K279C | TGT | L345N | AAT |
| F014T | ACT | P080N | AAT | L146K | AAG | D212Q | CAG | K279D | GAT | L345P | CCT |
| F014V | GTG | P080R | AGG | L146N | AAT | D212S | TCG | K279F | TTT | L345Q | CAG |
| F014W | TGG | P080S | TCT | L146P | CCT | D212T | ACT | K279G | GGG | L345R | CGT |
| L015A | GCG | P080T | ACG | L146Q | CAG | D212V | GTG | K279H | CAT | L345T | ACT |
| L015E | GAG | P080V | GTG | L146R | CGG | D212W | TGG | K279L | CTG | L345V | GTT |
| L015F | TTT | P080Y | TAT | L146S | TCG | D213A | GCT | K279P | CCT | L345W | TGG |
| L015G | GGG | Q081A | GCT | L146T | ACT | D213E | GAG | K279Q | CAG | L345Y | TAT |
| L015K | AAG | Q081C | TGT | L146V | GTT | D213G | GGG | K279R | AGG | C346A | GCT |
| L015M | ATG | Q081E | GAG | L146Y | TAT | D213H | CAT | K279S | TCT | C346D | GAT |
| L015N | AAT | Q081F | TTT | T147A | GCT | D213K | AAG | K279T | ACG | C346F | TTT |
| L015P | CCG | Q081G | GGG | T147C | TGT | D213L | CTG | K279V | GTG | C346G | GGG |
| L015Q | CAG | Q081H | CAT | T147D | GAT | D213M | ATG | K279W | TGG | C346I | ATT |
| L015R | CGG | Q081L | CTG | T147F | TTT | D213N | AAT | K279Y | TAT | C346K | AAG |
| L015S | TCG | Q081M | ATG | T147G | GGT | D213P | CCT | F280D | GAT | C346L | CTT |
| L015T | ACT | Q081N | AAT | T147I | ATT | D213Q | CAG | F280E | GAG | C346M | ATG |
| L015V | GTT | Q081P | CCG | T147L | CTT | D213R | CGT | F280G | GGG | C346P | CCT |
| L015W | TGG | Q081R | AGG | T147M | ATG | D213S | TCG | F280H | CAT | C346Q | CAG |
| L015Y | TAT | Q081S | TCT | T147P | CCT | D213V | GTG | F280I | ATT | C346R | CGG |
| W016A | GCG | Q081V | GTT | T147Q | CAG | D213W | TGG | F280L | TTG | C346S | TCT |
| W016C | TGT | Q081W | TGG | T147R | CGT | D213Y | TAT | F280M | ATG | C346T | ACT |
| W016D | GAT | Q081Y | TAT | T147S | AGT | L214A | GCG | F280N | AAT | C346V | GTG |
| W016E | GAG | K082A | GCT | T147V | GTT | L214C | TGT | F280P | CCT | C346W | TGG |
| W016F | TTT | K082E | GAG | T147W | TGG | L214D | GAT | F280Q | CAG | Q347A | GCT |
| W016G | GGT | K082G | GGT | T147Y | TAT | L214E | GAG | F280R | CGT | Q347C | TGT |
| W016H | CAT | K082H | CAT | E148C | TGT | L214G | GGG | F280S | TCG | Q347E | GAG |
| W016K | AAG | K082I | ATT | E148F | TTT | L214H | CAT | F280T | ACT | Q347F | TTT |
| W016L | CTT | K082L | CTT | E148G | GGG | L214K | AAG | F280V | GTG | Q347G | GGT |
| W016M | ATG | K082M | ATG | E148H | CAT | L214N | AAT | F280W | TGG | Q347I | ATT |
| W016P | CCT | K082N | AAT | E148I | ATT | L214P | CCG | L281A | GCG | Q347L | TTG |
| W016R | CGT | K082P | CCT | E148K | AAG | L214Q | CAG | L281D | GAT | Q347M | ATG |
| W016S | TCG | K082Q | CAG | E148L | CTG | L214R | CGG | L281F | TTT | Q347P | CCT |
| W016T | ACT | K082R | CGT | E148P | CCT | L214S | TCG | L281G | GGT | Q347R | AGG |
| W016Y | TAT | K082S | AGT | E148Q | CAG | L214T | ACG | L281H | CAT | Q347S | TCT |
| A017D | GAT | K082T | ACT | E148R | CGG | L214V | GTG | L281I | ATT | Q347T | ACT |
| A017E | GAG | K082V | GTG | E148S | TCT | L214Y | TAT | L281K | AAG | Q347V | GTG |
| A017G | GGG | K082W | TGG | E148T | ACT | S215A | GCT | L281N | AAT | Q347W | TGG |
| A017H | CAT | K082Y | TAT | E148V | GTG | S215C | TGT | L281P | CCG | Q347Y | TAT |
| A017I | ATT | I083E | GAG | E148W | TGG | S215D | GAT | L281Q | CAG | E348C | TGT |
| A017L | CTT | I083F | TTT | E148Y | TAT | S215E | GAG | L281R | CGG | E348D | GAT |
| A017N | AAT | I083G | GGT | A149C | TGT | S215G | GGG | L281S | AGT | E348G | GGT |
| A017P | CCG | I083H | CAT | A149E | GAG | S215H | CAT | L281V | GTT | E348H | CAT |
| A017Q | CAG | I083K | AAG | A149F | TTT | S215K | AAG | L281W | TGG | E348I | ATT |
| A017R | AGG | I083L | CTG | A149G | GGT | S215L | TTG | L281Y | TAT | E348L | TTG |
| A017S | TCG | I083N | AAT | A149K | AAG | S215M | ATG | S282A | GCG | E348M | ATG |
| A017T | ACG | I083P | CCT | A149L | TTG | S215P | CCG | S282C | TGT | E348P | CCT |
| A017V | GTG | I083Q | CAA | A149M | ATG | S215Q | CAG | S282D | GAT | E348Q | CAG |
| A017W | TGG | I083R | CGT | A149P | CCT | S215R | CGG | S282E | GAG | E348R | CGG |
| A017Y | TAT | I083S | TCG | A149Q | CAG | S215T | ACT | S282F | TTT | E348S | TCT |
| W018C | TGT | I083T | ACT | A149R | CGG | S215V | GTG | S282G | GGT | E348T | ACT |
| W018D | GAT | I083V | GTT | A149S | TCT | S215W | TGG | S282L | CTT | E348V | GTT |
| W018F | TTT | I083Y | TAT | A149T | ACT | W216D | GAT | S282M | ATG | E348W | TGG |
| W018G | GGG | S084D | GAT | A149V | GTT | W216E | GAG | S282P | CCT | E348Y | TAT |
| W018H | CAT | S084E | GAG | A149W | TGG | W216G | GGT | S282Q | CAG | Q349A | GCT |
| W018I | ATT | S084F | TTT | A149Y | TAT | W216H | CAT | S282R | CGT | Q349D | GAT |
| W018L | CTG | S084G | GGT | T150A | GCT | W216I | ATT | S282T | ACT | Q349E | GAG |
| W018M | ATG | S084H | CAT | T150C | TGT | W216K | AAG | S282V | GTT | Q349F | TTT |
| W018P | CCG | S084I | ATT | T150D | GAT | W216L | CTG | S282W | TGG | Q349G | GGT |
| W018Q | CAG | S084L | CTT | T150E | GAG | W216M | ATG | S282Y | TAT | Q349H | CAT |
| W018R | CGG | S084M | ATG | T150F | TTT | W216N | AAT | Q283A | GCG | Q349K | AAG |
| W018S | AGT | S084N | AAT | T150G | GGG | W216P | CCT | Q283C | TGT | Q349L | CTG |
| W018T | ACG | S084P | CCT | T150I | ATT | W216Q | CAG | Q283D | GAT | Q349M | ATG |
| W018V | GTG | S084Q | CAG | T150L | TTG | W216R | CGG | Q283E | GAG | Q349N | AAT |
| W018Y | TAT | S084R | CGG | T150N | AAT | W216T | ACG | Q283F | TTT | Q349P | CCT |
| N019A | GCG | S084T | ACT | T150P | CCT | W216V | GTG | Q283G | GGG | Q349R | CGT |
| N019C | TGT | S084W | TGG | T150R | AGG | W216Y | TAT | Q283H | CAT | Q349S | TCG |
| N019F | TTT | S084Y | TAT | T150S | TCT | L217A | GCG | Q283L | CTT | Q349T | ACT |
| N019G | GGG | L085A | GCT | T150V | GTG | L217C | TGT | Q283N | AAT | Q349V | GTG |
| N019H | CAT | L085C | TGT | T150W | TGG | L217E | GAG | Q283P | CCG | Q349W | TGG |
| N019I | ATT | L085D | GAT | T150Y | TAT | L217G | GGT | Q283R | CGT | Q349Y | TAT |
| N019L | CTG | L085E | GAG | E151A | GCT | L217H | CAT | Q283S | TCT | G350A | GCT |
| N019M | ATG | L085F | TTT | E151C | TGT | L217I | ATT | Q283T | ACT | G350D | GAT |
| N019P | CCG | L085G | GGG | E151G | GGT | L217M | ATG | Q283W | TGG | G350E | GAG |
| N019Q | CAG | L085H | CAT | E151H | CAT | L217P | CCG | Q283Y | TAT | G350F | TTT |
| N019R | CGT | L085K | AAG | E151K | AAG | L217Q | CAG | D284A | GCT | G350H | CAT |
| N019S | TCG | L085N | AAT | E151L | TTG | L217R | AGG | D284C | TGT | G350K | AAG |
| N019V | GTT | L085P | CCT | E151M | ATG | L217S | TCT | D284E | GAG | G350L | CTG |
| N019W | TGG | L085Q | CAG | E151N | AAT | L217T | ACG | D284G | GGT | G350M | ATG |
| N019Y | TAT | L085R | CGT | E151Q | CAG | L217V | GTG | D284H | CAT | G350N | AAT |
| A020D | GAT | L085S | TCG | E151R | AGG | L217W | TGG | D284I | ATT | G350P | CCT |
| A020E | GAG | L085T | ACT | E151S | TCG | L217Y | TAT | D284L | TTG | G350R | CGT |
| A020F | TTT | L085V | GTT | E151T | ACT | W218A | GCT | D284M | ATG | G350S | TCT |
| A020G | GGG | Q086A | GCT | E151V | GTT | W218D | GAT | D284N | AAT | G350T | ACT |
| A020H | CAT | Q086C | TGT | E151W | TGG | W218F | TTT | D284P | CCG | G350V | GTG |
| A020K | AAG | Q086D | GAT | E151Y | TAT | W218G | GGT | D284Q | CAG | G350Y | TAT |
| A020L | CTG | Q086E | GAG | K152A | GCT | W218H | CAT | D284S | TCT | V351A | GCT |
| A020N | AAT | Q086F | TTT | K152C | TGT | W218I | ATT | D284T | ACG | V351C | TGT |
| A020P | CCG | Q086G | GGT | K152F | TTT | W218K | AAG | D284V | GTT | V351D | GAT |
| A020Q | CAG | Q086H | CAT | K152G | GGT | W218L | CTT | D284Y | TAT | V351E | GAG |
| A020R | CGT | Q086I | ATT | K152I | ATT | W218M | ATG | E285A | GCG | V351F | TTT |
| A020S | TCT | Q086K | AAG | K152L | TTG | W218P | CCT | E285F | TTT | V351G | GGT |
| A020T | ACT | Q086L | CTG | K152M | ATG | W218Q | CAG | E285G | GGG | V351H | CAT |
| A020V | GTT | Q086M | ATG | K152N | AAT | W218R | CGG | E285H | CAT | V351I | ATT |
| A020Y | TAT | Q086N | AAT | K152P | CCT | W218S | TCG | E285K | AAG | V351L | TTG |
| P021A | GCG | Q086P | CCT | K152R | AGG | W218T | ACT | E285M | ATG | V351N | AAT |
| P021C | TGT | Q086R | CGG | K152S | TCT | W218V | GTG | E285N | AAT | V351Q | CAG |
| P021D | GAT | Q086S | TCT | K152T | ACT | N219A | GCG | E285P | CCT | V351R | AGG |
| P021E | GAG | Q086T | ACT | K152V | GTG | N219C | TGT | E285Q | CAG | V351S | TCT |
| P021G | GGG | Q086V | GTG | K152W | TGG | N219D | GAT | E285R | CGT | V351W | TGG |
| P021H | CAT | Q086W | TGG | K152Y | TAT | N219E | GAG | E285S | AGT | V351Y | TAT |
| P021I | ATT | D087A | GCT | A153C | TGT | N219G | GGG | E285T | ACG | C352A | GCT |
| P021K | AAG | D087C | TGT | A153E | GAG | N219H | CAT | E285V | GTG | C352D | GAT |
| P021L | CTT | D087E | GAG | A153F | TTT | N219I | ATT | E285W | TGG | C352E | GAG |
| P021M | ATG | D087G | GGG | A153G | GGT | N219K | AAG | E285Y | TAT | C352F | TTT |
| P021R | CGT | D087H | CAT | A153H | CAT | N219L | CTT | L286A | GCG | C352G | GGG |
| P021S | TCT | D087I | ATT | A153I | ATT | N219M | ATG | L286C | TGT | C352K | AAG |
| P021T | ACG | D087L | CTG | A153K | AAG | N219P | CCT | L286D | GAT | C352M | ATG |
| P021V | GTT | D087M | ATG | A153L | CTG | N219R | CGT | L286E | GAG | C352P | CCT |
| P021W | TGG | D087P | CCT | A153M | ATG | N219S | TCG | L286F | TTT | C352Q | CAG |
| S022A | GCT | D087Q | CAG | A153P | CCT | N219T | ACT | L286G | GGT | C352R | CGT |
| S022C | TGT | D087R | AGG | A153Q | CAG | N219W | TGG | L286H | CAT | C352S | AGT |
| S022D | GAT | D087S | TCG | A153R | CGT | E220A | GCG | L286K | AAG | C352T | ACT |
| S022E | GAG | D087T | ACT | A153S | AGT | E220D | GAT | L286M | ATG | C352V | GTG |
| S022G | GGG | D087V | GTT | A153T | ACT | E220G | GGG | L286P | CCT | C352W | TGG |
| S022H | CAT | D087Y | TAT | A153V | GTG | E220H | CAT | L286R | AGG | C352Y | TAT |
| S022K | AAG | H088A | GCT | A153W | TGG | E220I | ATT | L286S | AGT | I353A | GCT |
| S022L | CTG | H088C | TGT | K154A | GCT | E220K | AAG | L286T | ACG | I353C | TGT |
| S022M | ATG | H088E | GAG | K154C | TGT | E220L | TTG | L286W | TGG | I353E | GAG |
| S022N | AAT | H088F | TTT | K154D | GAT | E220M | ATG | L286Y | TAT | I353F | TTT |
| S022P | CCG | H088G | GGG | K154E | GAG | E220N | AAT | V287A | GCT | I353G | GGG |
| S022R | CGG | H088I | ATT | K154G | GGT | E220P | CCG | V287C | TGT | I353H | CAT |
| S022T | ACT | H088K | AAG | K154H | CAT | E220R | CGG | V287D | GAT | I353K | AAG |
| S022V | GTG | H088L | TTG | K154I | ATT | E220S | TCT | V287E | GAG | I353L | CTT |
| S022Y | TAT | H088M | ATG | K154L | CTG | E220T | ACG | V287F | TTT | I353M | ATG |
| E023A | GCT | H088P | CCT | K154P | CCT | E220V | GTG | V287G | GGG | I353Q | CAG |
| E023D | GAT | H088R | CGT | K154R | CGG | E220W | TGG | V287I | ATT | I353R | CGT |
| E023F | TTT | H088S | AGT | K154S | AGT | S221A | GCG | V287K | AAG | I353S | TCG |
| E023G | GGG | H088T | ACT | K154T | ACT | S221C | TGT | V287L | CTT | I353T | ACT |
| E023H | CAT | H088V | GTT | K154V | GTG | S221D | GAT | V287N | AAT | I353V | GTG |
| E023L | CTT | H088Y | TAT | K154W | TGG | S221E | GAG | V287P | CCT | I353W | TGG |
| E023M | ATG | L089A | GCT | K154Y | TAT | S221G | GGG | V287Q | CAG | R354C | TGT |
| E023N | AAT | L089C | TGT | Q155A | GCT | S221H | CAT | V287R | CGG | R354D | GAT |
| E023P | CCT | L089D | GAT | Q155C | TGT | S221I | ATT | V287S | TCT | R354E | GAG |
| E023Q | CAG | L089E | GAG | Q155D | GAT | S221K | AAG | V287T | ACT | R354G | GGT |
| E023R | CGG | L089G | GGG | Q155F | TTT | S221L | TTG | Y288D | GAC | R354H | CAT |
| E023S | TCT | L089K | AAG | Q155G | GGG | S221M | ATG | Y288E | GAG | R354I | ATT |
| E023T | ACG | L089M | ATG | Q155H | CAT | S221P | CCG | Y288F | TTT | R354K | AAG |
| E023V | GTG | L089N | AAT | Q155K | AAG | S221Q | CAG | Y288G | GGG | R354L | CTT |
| E023W | TGG | L089P | CCT | Q155L | CTT | S221R | CGG | Y288H | CAT | R354M | ATG |
| F024A | GCG | L089Q | CAG | Q155M | ATG | S221T | ACT | Y288I | ATT | R354P | CCT |
| F024C | TGT | L089R | AGG | Q155P | CCT | S221V | GTG | Y288K | AAG | R354Q | CAG |
| F024E | GAG | L089S | TCG | Q155R | CGG | T222A | GCG | Y288L | CTG | R354S | TCT |
| F024G | GGG | L089T | ACT | Q155S | AGT | T222D | GAT | Y288P | CCT | R354V | GTG |
| F024H | CAT | L089W | TGG | Q155T | ACT | T222E | GAG | Y288Q | CAG | R354W | TGG |
| F024I | ATT | L089Y | TAT | Q155V | GTT | T222F | TTT | Y288R | CGT | R354Y | TAT |
| F024K | AAG | D090A | GCT | Q155W | TGG | T222G | GGG | Y288S | TCT | K355D | GAT |
| F024L | TTG | D090C | TGT | Q155Y | TAT | T222I | ATT | Y288T | ACT | K355F | TTT |
| F024M | ATG | D090E | GAG | E156A | GCT | T222K | AAA | Y288V | GTG | K355G | GGG |
| F024N | AAT | D090G | GGG | E156C | TGT | T222L | TTG | Y288W | TGG | K355H | CAT |
| F024P | CCT | D090H | CAT | E156D | GAT | T222N | AAT | T289A | GCT | K355L | CTG |
| F024R | CGT | D090I | ATT | E156G | GGT | T222P | CCG | T289C | TGT | K355M | ATG |
| F024T | ACG | D090K | AAG | E156I | ATT | T222R | CGG | T289E | GAG | K355N | AAT |
| F024V | GTT | D090L | CTT | E156K | AAG | T222S | AGT | T289G | GGT | K355P | CCT |
| F024Y | TAT | D090N | AAT | E156L | CTG | T222V | GTT | T289H | CAT | K355Q | CAG |
| C025D | GAT | D090P | CCT | E156M | ATG | T222W | TGG | T289K | AAG | K355R | CGT |
| C025E | GAG | D090Q | CAG | E156P | CCT | T222Y | TAT | T289L | CTT | K355S | TCT |
| C025F | TTT | D090R | AGG | E156Q | CAG | A223C | TGT | T289M | ATG | K355T | ACT |
| C025G | GGG | D090S | AGT | E156R | CGG | A223D | GAT | T289N | AAT | K355V | GTG |
| C025H | CAT | D090T | ACT | E156S | TCT | A223E | GAG | T289P | CCT | K355W | TGG |
| C025I | ATT | D090W | TGG | E156T | ACT | A223G | GGG | T289Q | CAG | K355Y | TAT |
| C025K | AAG | K091A | GCT | E156V | GTT | A223H | CAT | T289R | AGG | N356A | GCT |
| C025L | TTG | K091D | GAT | E156W | TGG | A223K | AAG | T289S | TCG | N356C | TGT |
| C025N | AAT | K091E | GAG | F157A | GCT | A223L | CTG | T289V | GTG | N356D | GAT |
| C025P | CCT | K091F | TTT | F157C | TGT | A223P | CCT | T289Y | TAT | N356F | TTT |
| C025R | CGT | K091G | GGG | F157D | GAT | A223Q | CAG | F290A | GCT | N356G | GGG |
| C025S | TCT | K091H | CAT | F157E | GAG | A223R | AGG | F290C | TGT | N356H | CAT |
| C025T | ACT | K091I | ATT | F157G | GGT | A223S | TCT | F290D | GAT | N356K | AAG |
| C025V | GTG | K091L | TTG | F157H | CAT | A223T | ACG | F290G | GGG | N356L | CTG |
| C025Y | TAT | K091N | AAT | F157I | ATT | A223V | GTG | F290H | CAT | N356P | CCT |
| L026A | GCT | K091Q | CAG | F157K | AAG | A223W | TGG | F290I | ATT | N356Q | CAG |
| L026E | GAG | K091R | CGT | F157L | TTG | A223Y | TAT | F290K | AAG | N356R | CGG |
| L026G | GGT | K091S | TCT | F157M | ATG | L224A | GCT | F290L | TTG | N356S | AGT |
| L026H | CAT | K091T | ACT | F157P | CCT | L224D | GAT | F290M | ATG | N356T | ACT |
| L026I | ATT | K091Y | TAT | F157Q | CAG | L224E | GAG | F290Q | CAG | N356V | GTG |
| L026K | AAG | A092C | TGT | F157R | CGG | L224F | TTT | F290R | AGG | N356W | TGG |
| L026M | ATG | A092E | GAG | F157S | TCG | L224G | GGG | F290S | TCG | W357A | GCT |
| L026P | CCG | A092F | TTT | F157T | ACT | L224I | ATT | F290T | ACT | W357C | TGT |
| L026Q | CAG | A092G | GGT | F157V | GTG | L224M | ATG | F290V | GTT | W357D | GAT |
| L026R | CGG | A092H | CAT | F157W | TGG | L224P | CCG | F290Y | TAT | W357E | GAG |
| L026S | TCT | A092K | AAG | E158A | GCT | L224Q | CAG | G291A | GCT | W357F | TTT |
| L026T | ACT | A092L | CTG | E158C | TGT | L224R | AGG | G291C | TGT | W357G | GGG |
| L026V | GTT | A092M | ATG | E158D | GAT | L224S | AGT | G291D | GAT | W357K | AAG |
| L026W | TGG | A092P | CCT | E158F | TTT | L224T | ACT | G291E | GAG | W357L | TTG |
| L026Y | TAT | A092Q | CAG | E158G | GGG | L224V | GTT | G291F | TTT | W357M | ATG |
| G027A | GCT | A092R | CGT | E158H | CAT | L224W | TGG | G291H | CAT | W357P | CCT |
| G027C | TGT | A092T | ACT | E158K | AAG | L224Y | TAT | G291L | CTG | W357Q | CAG |
| G027D | GAT | A092V | GTT | E158L | CTG | Y225A | GCG | G291M | ATG | W357R | CGT |
| G027E | GAG | A092W | TGG | E158N | AAT | Y225D | GAT | G291N | AAT | W357S | AGT |
| G027F | TTT | A092Y | TAT | E158P | CCT | Y225E | GAG | G291P | CCT | W357T | ACT |
| G027H | CAT | K093D | GAT | E158Q | CAG | Y225G | GGT | G291Q | CAG | W357V | GTG |
| G027I | ATT | K093E | GAG | E158R | CGG | Y225H | CAT | G291R | CGG | N358C | TGT |
| G027K | AAG | K093F | TTT | E158S | TCG | Y225K | AAG | G291S | TCT | N358D | GAT |
| G027L | CTG | K093G | GGT | E158V | GTG | Y225L | CTG | G291T | ACT | N358E | GAG |
| G027P | CCT | K093H | CAT | E158Y | TAT | Y225P | CCG | G291V | GTG | N358G | GGG |
| G027Q | CAG | K093I | ATT | K159A | GCT | Y225Q | CAG | G291W | TGG | N358H | CAT |
| G027R | CGG | K093L | CTG | K159D | GAT | Y225R | AGG | G291Y | TAT | N358I | ATT |
| G027S | TCG | K093M | ATG | K159E | GAG | Y225S | TCT | E292A | GCT | N358K | AAG |
| G027T | ACT | K093N | AAT | K159F | TTT | Y225T | ACG | E292C | TGT | N358L | CTG |
| G027W | TGG | K093P | CCT | K159G | GGT | Y225V | GTG | E292F | TTT | N358P | CCT |
| K028A | GCG | K093Q | CAG | K159H | CAT | Y225W | TGG | E292G | GGT | N358Q | CAG |
| K028D | GAT | K093R | CGG | K159L | CTT | P226A | GCG | E292H | CAT | N358R | CGT |
| K028E | GAG | K093S | AGT | K159M | ATG | P226C | TGT | E292I | ATT | N358S | TCT |
| K028F | TTT | K093T | ACT | K159N | AAT | P226D | GAT | E292K | AAG | N358T | ACT |
| K028G | GGG | K093V | GTT | K159Q | CAG | P226E | GAG | E292L | TTG | N358V | GTG |
| K028I | ATT | K094A | GCT | K159R | CGG | P226F | TTT | E292N | AAT | N358W | TGG |
| K028L | TTG | K094C | TGT | K159S | TCT | P226G | GGT | E292P | CCT | S359A | GCT |
| K028M | ATG | K094D | GAT | K159V | GTG | P226L | CTT | E292Q | CAG | S359C | TGT |
| K028N | AAT | K094E | GAG | K159W | TGG | P226N | AAT | E292R | CGG | S359D | GAT |
| K028P | CCT | K094F | TTT | K159Y | TAT | P226Q | CAG | E292T | ACT | S359E | GAG |
| K028R | CGG | K094G | GGG | A160C | TGT | P226R | AGG | E292V | GTT | S359F | TTT |
| K028S | AGT | K094H | CAT | A160F | TTT | P226S | TCT | E292W | TGG | S359G | GGG |
| K028T | ACT | K094L | TTG | A160G | GGG | P226T | ACG | T293A | GCT | S359H | CAT |
| K028V | GTT | K094M | ATG | A160H | CAT | P226V | GTT | T293C | TGT | S359K | AAG |
| K028W | TGG | K094N | AAT | A160I | ATT | P226W | TGG | T293D | GAT | S359L | TTG |
| F029A | GCT | K094P | CCT | A160K | AAG | P226Y | TAT | T293E | GAG | S359M | ATG |
| F029C | TGT | K094Q | CAG | A160L | CTG | S227A | GCT | T293F | TTT | S359P | CCT |
| F029E | GAG | K094R | AGG | A160M | ATG | S227F | TTT | T293G | GGT | S359R | CGG |
| F029G | GGG | K094S | TCT | A160N | AAT | S227G | GGG | T293K | AAG | S359T | ACT |
| F029H | CAT | K094T | ACT | A160Q | CAG | S227H | CAT | T293L | CTT | S359V | GTT |
| F029I | ATT | D095A | GCT | A160R | AGG | S227I | ATT | T293M | ATG | S359W | TGG |
| F029K | AAG | D095C | TGT | A160S | AGT | S227K | AAG | T293N | AAT | S360A | GCT |
| F029L | CTT | D095E | GAG | A160V | GTG | S227L | TTG | T293P | CCT | S360C | TGT |
| F029M | ATG | D095F | TTT | A160W | TGG | S227M | ATG | T293Q | CAG | S360E | GAG |
| F029P | CCG | D095G | GGG | A160Y | TAT | S227P | CCT | T293S | TCT | S360F | TTT |
| F029R | CGG | D095H | CAT | G161A | GCT | S227Q | CAG | T293V | GTG | S360G | GGG |
| F029S | TCG | D095K | AAG | G161C | TGT | S227R | CGG | T293Y | TAT | S360I | ATT |
| F029T | ACG | D095L | TTG | G161D | GAT | S227T | ACG | V294A | GCT | S360K | AAG |
| F029V | GTG | D095M | ATG | G161E | GAG | S227V | GTG | V294C | TGT | S360L | CTG |
| F029W | TGG | D095P | CCT | G161H | CAT | S227W | TGG | V294E | GAG | S360M | ATG |
| D030A | GCG | D095Q | CAG | G161I | ATT | S227Y | TAT | V294G | GGG | S360N | AAT |
| D030E | GAG | D095S | TCT | G161K | AAG | I228A | GCG | V294H | CAT | S360P | CCT |
| D030F | TTT | D095V | GTG | G161L | CTT | I228E | GAG | V294K | AAG | S360Q | CAG |
| D030G | GGG | D095W | TGG | G161M | ATG | I228F | TTT | V294L | TTG | S360R | AGG |
| D030H | CAT | D095Y | TAT | G161Q | CAG | I228G | GGG | V294M | ATG | S360T | ACT |
| D030K | AAG | I096A | GCT | G161R | CGT | I228H | CAT | V294N | AAT | S360V | GTT |
| D030L | TTG | I096C | TGT | G161S | AGT | I228K | AAG | V294P | CCT | D361A | GCT |
| D030M | ATG | I096D | GAT | G161T | ACT | I228L | TTG | V294Q | CAG | D361C | TGT |
| D030P | CCT | I096E | GAG | G161V | GTG | I228M | ATG | V294R | AGG | D361E | GAG |
| D030Q | CAG | I096F | TTT | G161W | TGG | I228N | AAT | V294S | AGT | D361G | GGG |
| D030R | CGG | I096G | GGG | K162A | GCT | I228P | CCG | V294T | ACT | D361H | CAT |
| D030S | TCG | I096H | CAT | K162D | GAT | I228Q | CAG | V294W | TGG | D361L | TTG |
| D030T | ACT | I096L | TTG | K162E | GAG | I228R | CGT | A295C | TGT | D361M | ATG |
| D030V | GTT | I096N | AAT | K162F | TTT | I228S | TCT | A295D | GAT | D361N | AAT |
| D030W | TGG | I096P | CCT | K162G | GGG | I228T | ACT | A295E | GAG | D361P | CCT |
| E031A | GCG | I096R | CGT | K162H | CAT | I228W | TGG | A295F | TTT | D361Q | CAG |
| E031C | TGT | I096S | AGT | K162L | TTG | Y229E | GAG | A295G | GGG | D361R | AGG |
| E031G | GGG | I096T | ACT | K162M | ATG | Y229F | TTT | A295H | CAT | D361S | TCG |
| E031H | CAT | I096V | GTG | K162P | CCT | Y229G | GGT | A295I | ATT | D361V | GTT |
| E031I | ATT | I096W | TGG | K162Q | CAG | Y229H | CAT | A295L | CTG | D361W | TGG |
| E031K | AAG | T097A | GCT | K162R | CGG | Y229I | ATT | A295N | AAT | D361Y | TAT |
| E031L | CTG | T097C | TGT | K162S | TCG | Y229K | AAG | A295P | CCT | Y362A | GCT |
| E031N | AAC | T097D | GAT | K162V | GTG | Y229L | TTG | A295Q | CAG | Y362C | TGT |
| E031P | CCG | T097E | GAG | K162W | TGG | Y229N | AAT | A295S | AGT | Y362E | GAG |
| E031R | CGG | T097F | TTT | K162Y | TAT | Y229P | CCT | A295T | ACT | Y362G | GGG |
| E031S | TCT | T097G | GGG | D163A | GCT | Y229Q | CAG | A295V | GTT | Y362H | CAT |
| E031T | ACG | T097I | ATT | D163C | TGT | Y229R | CGT | A295Y | TAT | Y362K | AAG |
| E031V | GTG | T097L | CTT | D163E | GAG | Y229S | TCG | L296A | GCT | Y362L | CTT |
| E031W | TGG | T097N | AAT | D163F | TTT | Y229T | ACT | L296C | TGT | Y362M | ATG |
| E031Y | TAT | T097P | CCT | D163G | GGG | Y229V | GTG | L296F | TTT | Y362N | AAT |
| P032A | GCG | T097Q | CAG | D163H | CAC | Y229W | TGG | L296G | GGT | Y362P | CCT |
| P032C | TGT | T097R | CGG | D163K | AAG | L230A | GCG | L296I | ATT | Y362R | CGG |
| P032F | TTT | T097S | TCG | D163L | CTT | L230E | GAG | L296K | AAG | Y362S | AGT |
| P032G | GGG | T097W | TGG | D163P | CCT | L230G | GGG | L296M | ATG | Y362T | ACT |
| P032H | CAT | T097Y | TAT | D163Q | CAG | L230H | CAT | L296P | CCT | Y362V | GTG |
| P032K | AAG | F098A | GCT | D163R | AGG | L230I | ATT | L296Q | CAG | Y362W | TGG |
| P032L | CTG | F098C | TGT | D163S | TCG | L230K | AAG | L296R | CGT | L363A | GCT |
| P032M | ATG | F098D | GAT | D163T | ACT | L230M | ATG | L296S | TCG | L363C | TGT |
| P032N | AAT | F098E | GAG | D163V | GTG | L230N | AAT | L296T | ACT | L363D | GAT |
| P032Q | CAG | F098G | GGG | D163W | TGG | L230P | CCT | L296V | GTT | L363E | GAG |
| P032R | CGG | F098H | CAT | F164A | GCT | L230R | CGT | L296W | TGG | L363F | TTT |
| P032S | TCG | F098I | ATT | F164C | TGT | L230S | AGT | L296Y | TAT | L363G | GGG |
| P032T | ACT | F098L | TTG | F164D | GAT | L230T | ACT | G297A | GCT | L363H | CAT |
| P032V | GTG | F098M | ATG | F164E | GAG | L230V | GTT | G297C | TGT | L363I | ATT |
| P032W | TGG | F098P | CCT | F164G | GGG | L230W | TGG | G297E | GAG | L363P | CCT |
| P032Y | TAT | F098Q | CAG | F164H | CAT | L230Y | TAT | G297H | CAT | L363Q | CAG |
| L033C | TGT | F098R | CGT | F164L | TTG | N231A | GCT | G297I | ATT | L363R | CGG |
| L033D | GAT | F098S | TCG | F164M | ATG | N231C | TGT | G297L | CTT | L363S | TCG |
| L033G | GGG | F098V | GTT | F164N | AAT | N231D | GAT | G297N | AAT | L363T | ACT |
| L033H | CAT | F098W | TGG | F164P | CCT | N231F | TTT | G297P | CCT | L363V | GTG |
| L033I | ATT | Y099A | GCT | F164Q | CAG | N231G | GGG | G297Q | CAG | L363W | TGG |
| L033M | ATG | Y099C | TGT | F164R | CGG | N231H | CAT | G297R | CGG | H364A | GCT |
| L033N | AAT | Y099E | GAG | F164S | AGT | N231I | ATT | G297S | AGT | H364C | TGT |
| L033P | CCG | Y099F | TTT | F164V | GTT | N231K | AAG | G297T | ACT | H364D | GAT |
| L033Q | CAG | Y099G | GGT | F164W | TGG | N231L | CTT | G297V | GTG | H364E | GAG |
| L033R | AGG | Y099I | ATT | L165A | GCT | N231P | CCT | G297W | TGG | H364F | TTT |
| L033S | TCG | Y099L | TTG | L165C | TGT | N231Q | CAG | G297Y | TAT | H364G | GGG |
| L033T | ACT | Y099N | AAT | L165D | GAT | N231R | CGT | A298C | TGT | H364K | AAG |
| L033V | GTT | Y099P | CCT | L165F | TTT | N231S | TCT | A298E | GAG | H364L | CTG |
| L033W | TGG | Y099Q | CAG | L165G | GGG | N231T | ACG | A298G | GGG | H364M | ATG |
| L033Y | TAT | Y099R | AGG | L165H | CAT | N231V | GTG | A298I | ATT | H364P | CCT |
| D034A | GCT | Y099S | TCG | L165N | AAT | T232A | GCG | A298L | TTG | H364R | CGG |
| D034E | GAG | Y099T | ACT | L165P | CCT | T232C | TGT | A298M | ATG | H364S | TCT |
| D034G | GGT | Y099V | GTT | L165Q | CAG | T232F | TTT | A298N | AAT | H364T | ACT |
| D034H | CAT | Y099W | TGG | L165R | CGG | T232G | GGG | A298P | CCT | H364V | GTG |
| D034I | ATT | M100C | TGT | L165S | TCG | T232H | CAT | A298Q | CAG | H364Y | TAT |
| D034K | AAG | M100E | GAG | L165T | ACT | T232K | AAG | A298R | CGT | L365A | GCT |
| D034L | CTT | M100F | TTT | L165V | GTG | T232L | CTT | A298S | TCG | L365C | TGT |
| D034N | AAT | M100G | GGT | L165W | TGG | T232M | ATG | A298T | ACT | L365D | GAT |
| D034P | CCT | M100K | AAG | L165Y | TAT | T232N | AAT | A298V | GTG | L365E | GAG |
| D034Q | CAG | M100L | CTG | V166A | GCT | T232P | CCG | A298W | TGG | L365G | GGG |
| D034R | CGT | M100N | AAT | V166C | TGT | T232Q | CAG | A298Y | TAT | L365I | ATT |
| D034S | AGT | M100P | CCT | V166D | GAT | T232R | AGG | S299A | GCT | L365M | ATG |
| D034T | ACG | M100Q | CAG | V166E | GAG | T232S | AGT | S299C | TGT | L365N | AAT |
| D034V | GTT | M100R | CGG | V166F | TTT | T232V | GTG | S299D | GAT | L365P | CCT |
| D034W | TGG | M100S | TCT | V166G | GGT | T232Y | TAT | S299E | GAG | L365Q | CAG |
| M035A | GCG | M100T | ACT | V166H | CAT | Q233A | GCG | S299F | TTT | L365R | CGG |
| M035D | GAT | M100V | GTT | V166L | CTT | Q233C | TGT | S299G | GGG | L365S | AGT |
| M035F | TTT | M100W | TGG | V166N | AAT | Q233D | GAT | S299H | CAT | L365T | ACT |
| M035G | GGG | M100Y | TAT | V166P | CCT | Q233F | TTT | S299I | ATT | L365V | GTG |
| M035H | CAT | P101A | GCT | V166Q | CAG | Q233G | GGG | S299L | CTT | L365W | TGG |
| M035I | ATT | P101C | TGT | V166R | CGG | Q233I | ATT | S299M | ATG | L365Y | TAT |
| M035L | TTG | P101F | TTT | V166T | ACT | Q233K | AAG | S299P | CCT | N366A | GCT |
| M035N | AAT | P101G | GGG | V166W | TGG | Q233L | CTG | S299Q | CAG | N366C | TGT |
| M035P | CCG | P101H | CAT | V166Y | TAT | Q233P | CCG | S299R | AGG | N366E | GAG |
| M035Q | CAG | P101I | ATT | E167A | GCT | Q233R | AGG | S299T | ACT | N366F | TTT |
| M035R | CGT | P101K | AAG | E167D | GAT | Q233S | TCG | S299Y | TAT | N366G | GGG |
| M035S | TCT | P101L | CTT | E167F | TTT | Q233T | ACG | G300A | GCT | N366K | AAG |
| M035T | ACT | P101M | ATG | E167G | GGT | Q233V | GTG | G300C | TGT | N366L | TTG |
| M035V | GTT | P101N | AAT | E167H | CAT | Q233W | TGG | G300D | GAT | N366M | ATG |
| M035Y | TAT | P101Q | CAG | E167K | AAG | Q233Y | TAT | G300E | GAG | N366P | CCT |
| S036A | GCG | P101R | AGG | E167L | TTG | Q234A | GCT | G300F | TTT | N366Q | CAG |
| S036C | TGT | P101S | TCT | E167M | ATG | Q234C | TGT | G300L | CTT | N366R | AGG |
| S036D | GAT | P101T | ACT | E167N | AAT | Q234D | GAT | G300M | ATG | N366S | TCT |
| S036F | TTT | P101Y | TAT | E167P | CCT | Q234E | GAG | G300N | AAT | N366T | ACT |
| S036G | GGT | V102A | GCT | E167R | AGG | Q234G | GGT | G300P | CCT | N366V | GTT |
| S036H | CAT | V102C | TGT | E167S | TCG | Q234H | CAT | G300Q | CAG | N366W | TGG |
| S036K | AAG | V102E | GAG | E167T | ACT | Q234L | CTT | G300R | AGG | P367A | GCT |
| S036L | TTG | V102G | GGT | E167V | GTT | Q234M | ATG | G300S | TCG | P367C | TGT |
| S036N | AAT | V102H | CAT | E167Y | TAT | Q234N | AAT | G300T | ACT | P367E | GAG |
| S036P | CCG | V102K | AAG | T168A | GCT | Q234P | CCG | G300V | GTT | P367F | TTT |
| S036R | CGG | V102L | TTG | T168C | TGT | Q234R | CGG | G300W | TGG | P367G | GGT |
| S036T | ACG | V102M | ATG | T168D | GAT | Q234S | AGT | I301A | GCT | P367H | CAT |
| S036V | GTT | V102N | AAT | T168E | GAG | Q234T | ACT | I301E | GAG | P367I | ATT |
| S036W | TGG | V102P | CCT | T168F | TTT | Q234V | GTG | I301G | GGG | P367K | AAG |
| S036Y | TAT | V102Q | CAG | T168G | GGG | Q234W | TGG | I301H | CAT | P367L | CTG |
| L037A | GCG | V102R | AGG | T168H | CAT | S235A | GCG | I301K | AAG | P367M | ATG |
| L037C | TGT | V102S | TCT | T168K | AAG | S235E | GAG | I301L | CTG | P367Q | CAG |
| L037E | GAG | V102T | ACT | T168L | CTG | S235F | TTT | I301M | ATG | P367R | CGT |
| L037F | TTT | V102W | TGG | T168P | CCT | S235G | GGG | I301N | AAT | P367S | TCG |
| L037G | GGG | D103A | GCT | T168R | CGG | S235H | CAT | I301P | CCT | P367V | GTT |
| L037I | ATT | D103E | GAG | T168S | TCT | S235K | AAG | I301Q | CAG | P367W | TGG |
| L037K | AAG | D103F | TTT | T168V | GTG | S235L | CTT | I301R | CGG | D368A | GCT |
| L037M | ATG | D103G | GGG | T168W | TGG | S235M | ATG | I301S | AGT | D368C | TGT |
| L037N | AAT | D103H | CAT | T168Y | TAT | S235P | CCT | I301V | GTT | D368E | GAG |
| L037P | CCT | D103I | ATT | I169A | GCT | S235Q | CAG | I301W | TGG | D368G | GGT |
| L037R | AGG | D103L | CTT | I169D | GAT | S235R | CGG | I301Y | TAT | D368H | CAT |
| L037S | TCT | D103N | AAT | I169F | TTT | S235T | ACG | V302C | TGT | D368K | AAG |
| L037T | ACG | D103Q | CAG | I169G | GGG | S235V | GTG | V302D | GAT | D368L | CTT |
| L037V | GTG | D103R | AGG | I169H | CAT | S235W | TGG | V302E | GAG | D368M | ATG |
| L037W | TGG | D103S | TCG | I169K | AAG | S235Y | TAT | V302F | TTT | D368P | CCT |
| F038A | GCG | D103T | ACT | I169L | TTG | P236A | GCT | V302G | GGT | D368R | CGT |
| F038C | TGT | D103V | GTT | I169N | AAT | P236C | TGT | V302H | CAT | D368S | AGT |
| F038E | GAG | D103W | TGG | I169P | CCT | P236E | GAG | V302I | ATT | D368T | ACT |
| F038G | GGG | D103Y | TAT | I169Q | CAG | P236G | GGG | V302L | TTG | D368V | GTT |
| F038K | AAG | N104A | GCT | I169R | CGG | P236H | CAT | V302M | ATG | D368W | TGG |
| F038L | CTT | N104C | TGT | I169S | TCG | P236I | ATT | V302P | CCT | D368Y | TAT |
| F038M | ATG | N104F | TTT | I169T | ACT | P236K | AAG | V302R | AGG | N369A | GCT |
| F038N | AAT | N104G | GGG | I169V | GTT | P236L | CTG | V302S | TCG | N369C | TGT |
| F038P | CCT | N104H | CAT | I169Y | TAT | P236N | AAT | V302T | ACT | N369E | GAG |
| F038Q | CAG | N104I | ATT | K170A | GCT | P236Q | CAG | V302W | TGG | N369F | TTT |
| F038R | AGG | N104K | AAG | K170C | TGT | P236R | CGT | V302Y | TAT | N369H | CAT |
| F038S | TCT | N104L | CTG | K170D | GAT | P236S | AGT | I303A | GCT | N369I | ATT |
| F038T | ACT | N104M | ATG | K170E | GAG | P236T | ACT | I303C | TGT | N369K | AAG |
| F038W | TGG | N104P | CCT | K170G | GGG | P236W | TGG | I303D | GAT | N369L | CTT |
| F038Y | TAT | N104R | AGG | K170I | ATT | P236Y | TAT | I303E | GAG | N369P | CCT |
| S039A | GCG | N104S | TCT | K170L | TTG | V237A | GCG | I303F | TTT | N369Q | CAG |
| S039C | TGT | N104T | ACT | K170M | ATG | V237C | TGT | I303G | GGT | N369R | CGG |
| S039D | GAT | N104V | GTT | K170N | AAT | V237E | GAG | I303K | AAG | N369S | TCG |
| S039F | TTT | N104W | TGG | K170P | CCT | V237F | TTT | I303L | TTG | N369T | ACT |
| S039G | GGT | L105A | GCT | K170Q | CAG | V237G | GGT | I303M | ATG | N369V | GTG |
| S039L | TTG | L105C | TGT | K170R | CGT | V237H | CAT | I303P | CCT | N369W | TGG |
| S039M | ATG | L105D | GAT | K170V | GTT | V237L | TTG | I303R | CGT | F370A | GCT |
| S039N | AAT | L105E | GAG | K170W | TGG | V237N | AAT | I303S | AGT | F370D | GAT |
| S039P | CCG | L105G | GGT | K170Y | TAT | V237P | CCT | I303V | GTG | F370E | GAG |
| S039Q | CAG | L105H | CAT | L171A | GCT | V237Q | CAG | I303W | TGG | F370G | GGG |
| S039R | CGT | L105I | ATT | L171C | TGT | V237R | CGG | I303Y | TAT | F370H | CAT |
| S039T | ACT | L105M | ATG | L171D | GAT | V237S | TCG | W304A | GCT | F370I | ATT |
| S039V | GTT | L105N | AAT | L171G | GGG | V237T | ACG | W304C | TGT | F370K | AAG |
| S039W | TGG | L105P | CCT | L171H | CAT | V237W | TGG | W304D | GAT | F370L | CTG |
| S039Y | TAT | L105Q | CAG | L171I | ATT | V237Y | TAT | W304G | GGT | F370N | AAT |
| F040A | GCG | L105R | CGG | L171M | ATG | A238D | GAT | W304I | ATT | F370P | CCT |
| F040D | GAT | L105S | TCT | L171N | AAT | A238E | GAG | W304L | CTG | F370Q | CAG |
| F040E | GAG | L105T | ACT | L171P | CCT | A238F | TTT | W304M | ATG | F370R | AGG |
| F040G | GGT | L105V | GTT | L171Q | CAG | A238G | GGT | W304N | AAT | F370S | TCT |
| F040I | ATT | L105W | TGG | L171R | CGT | A238H | CAT | W304P | CCT | F370V | GTG |
| F040K | AAG | G106A | GCT | L171S | AGT | A238K | AAG | W304Q | CAG | F370Y | TAT |
| F040L | CTG | G106C | TGT | L171V | GTG | A238L | CTT | W304R | CGG | A371C | TGT |
| F040N | AAT | G106D | GAT | L171W | TGG | A238P | CCG | W304S | AGT | A371E | GAG |
| F040Q | CAG | G106E | GAG | L171Y | TAT | A238Q | CAG | W304T | ACT | A371F | TTT |
| F040R | CGG | G106F | TTT | G172A | GCT | A238R | AGG | W304V | GTG | A371G | GGG |
| F040S | TCT | G106H | CAT | G172C | TGT | A238S | AGT | W304Y | TAT | A371H | CAT |
| F040T | ACT | G106I | ATT | G172D | GAT | A238T | ACG | G305C | TGT | A371I | ATT |
| F040V | GTT | G106L | CTG | G172E | GAG | A238V | GTG | G305D | GAT | A371K | AAG |
| F040W | TGG | G106M | ATG | G172I | ATT | A238W | TGG | G305E | GAG | A371L | CTT |
| F040Y | TAT | G106N | AAT | G172L | CTT | A238Y | TAT | G305F | TTT | A371M | ATG |
| I041A | GCG | G106P | CCT | G172M | ATG | A239C | TGT | G305H | CAT | A371P | CCT |
| I041C | TGT | G106S | AGT | G172P | CCT | A239F | TTT | G305K | AAG | A371R | CGT |
| I041D | GAT | G106V | GTG | G172Q | CAG | A239G | GGT | G305L | CTT | A371S | TCG |
| I041E | GAG | G106W | TGG | G172R | CGT | A239H | CAT | G305N | AAT | A371T | ACT |
| I041F | TTT | G106Y | TAT | G172S | TCT | A239I | ATT | G305P | CCT | A371V | GTG |
| I041G | GGG | M107A | GCT | G172T | ACT | A239K | AAG | G305Q | CAG | A371W | TGG |
| I041H | CAT | M107C | TGT | G172V | GTT | T240K | AAG | G305R | CGT | I372A | GCT |
| I041N | AAT | M107D | GAT | G172W | TGG | A239L | TTG | G305S | TCG | I372D | GAT |
| I041P | CCG | M107F | TTT | G172Y | TAT | A239N | AAT | G305T | ACT | I372E | GAG |
| I041Q | CAG | M107G | GGG | K173D | GAT | A239P | CCT | G305V | GTG | I372F | TTT |
| I041R | AGG | M107H | CAT | K173E | GAG | A239R | AGG | G305Y | TAT | I372G | GGT |
| I041S | TCT | M107I | ATT | K173G | GGG | A239S | TCT | T306A | GCT | I372H | CAT |
| I041T | ACG | M107K | AAG | K173H | CAT | A239T | ACT | T306C | TGT | I372K | AAG |
| I041V | GTT | M107L | CTT | K173I | ATT | A239V | GTT | T306D | GAT | I372L | CTG |
| I041W | TGG | M107P | CCT | K173L | CTT | A239W | TGG | T306E | GAG | I372N | AAT |
| G042A | GCT | M107Q | CAG | K173M | ATG | A239Y | TAT | T306F | TTT | I372P | CCT |
| G042C | TGT | M107R | CGT | K173N | AAT | T240A | GCG | T306G | GGT | I372R | CGG |
| G042D | GAT | M107S | TCT | K173P | CCT | T240E | GAG | T306H | CAT | I372S | TCT |
| G042E | GAG | M107V | GTT | K173Q | CAG | T240F | TTT | T306I | ATT | I372T | ACT |
| G042H | CAT | M107W | TGG | K173R | CGG | T240G | GGG | T306L | CTG | I372V | GTG |
| G042I | ATT | A108D | GAT | K173S | TCG | T240L | CTT | T306P | CCT | I372W | TGG |
| G042K | AAG | A108E | GAG | K173V | GTG | T240M | ATG | T306R | AGG | Q373A | GCT |
| G042L | CTG | A108F | TTT | K173W | TGG | T240N | AAT | T306S | AGT | Q373C | TGT |
| G042M | ATG | A108G | GGT | K173Y | TAT | T240P | CCT | T306V | GTG | Q373E | GAG |
| G042P | CCT | A108H | CAT | L174A | GCT | T240Q | CAG | T306W | TGG | Q373F | TTT |
| G042Q | CAG | A108K | AAG | L174C | TGT | T240R | CGT | T306Y | TAT | Q373G | GGT |
| G042R | CGG | A108L | TTG | L174G | GGG | T240S | AGT | L307C | TGT | Q373H | CAT |
| G042S | TCT | A108M | ATG | L174H | CAT | T240V | GTG | L307E | GAG | Q373K | AAG |
| G042T | ACT | A108N | AAT | L174K | AAG | T240W | TGG | L307F | TTT | Q373L | CTG |
| G042V | GTT | A108P | CCT | L174M | ATG | T240Y | TAT | L307G | GGG | Q373M | ATG |
| S043A | GCG | A108Q | CAG | L174N | AAT | L241A | GCG | L307I | ATT | Q373N | AAT |
| S043D | GAT | A108R | CGG | L174P | CCT | L241C | TGT | L307K | AAG | Q373P | CCT |
| S043E | GAG | A108S | TCT | L174Q | CAG | L241D | GAT | L307N | AAT | Q373R | CGT |
| S043F | TTT | A108T | ACT | L174R | CGT | L241E | GAG | L307P | CCT | Q373S | TCT |
| S043G | GGT | A108V | GTG | L174S | TCG | L241F | TTT | L307Q | CAG | Q373T | ACT |
| S043H | CAT | A108Y | TAT | L174T | ACT | L241G | GGG | L307R | AGG | Q373V | GTT |
| S043I | ATT | V109A | GCT | L174V | GTT | L241I | ATT | L307S | AGT | Q373W | TGG |
| S043K | AAG | V109C | TGT | L174W | TGG | L241K | AAG | L307T | ACT | L374A | GCT |
| S043L | CTT | V109D | GAT | L174Y | TAT | L241P | CCT | L307V | GTG | L374D | GAT |
| S043N | AAT | V109E | GAG | L175C | TGT | L241Q | CAG | L307W | TGG | L374E | GAG |
| S043P | CCT | V109F | TTT | L175D | GAT | L241R | CGG | L307Y | TAT | L374G | GGT |
| S043Q | CAG | V109G | GGG | L175E | GAG | L241S | TCT | S308C | TGT | L374H | CAT |
| S043R | CGG | V109H | CAT | L175F | TTT | L241T | ACG | S308D | GAT | L374I | ATT |
| S043T | ACT | V109L | TTG | L175G | GGG | L241V | GTT | S308F | TTT | L374M | ATG |
| S043V | GTG | V109M | ATG | L175H | CAT | L241W | TGG | S308G | GGT | L374N | AAT |
| P044A | GCT | V109P | CCT | L175K | AAG | Y242A | GCG | S308H | CAT | L374P | CCT |
| P044C | TGT | V109Q | CAG | L175N | AAT | Y242C | TGT | S308K | AAG | L374R | AGG |
| P044E | GAG | V109R | AGG | L175P | CCT | Y242D | GAT | S308L | CTG | L374S | AGT |
| P044F | TTT | V109T | ACT | L175R | CGT | Y242F | TTT | S308M | ATG | L374T | ACT |
| P044G | GGG | V109W | TGG | L175S | TCT | Y242G | GGT | S308N | AAT | L374V | GTG |
| P044H | CAT | V109Y | TAT | L175T | ACT | Y242I | ATT | S308P | CCT | L374W | TGG |
| P044I | ATT | I110A | GCT | L175V | GTG | Y242K | AAG | S308R | CGG | L374Y | TAT |
| P044L | CTT | I110C | TGT | L175W | TGG | Y242L | CTT | S308T | ACT | E375A | GCT |
| P044N | AAT | I110D | GAT | L175Y | TAT | Y242M | ATG | S308V | GTT | E375C | TGT |
| P044Q | CAG | I110F | TTT | R176A | GCT | Y242P | CCG | S308W | TGG | E375F | TTT |
| P044R | CGT | I110G | GGG | R176C | TGT | Y242R | CGG | S308Y | TAT | E375G | GGT |
| P044S | TCT | I110H | CAT | R176E | GAG | Y242S | TCT | I309D | GAT | E375I | ATT |
| P044T | ACT | I110K | AAG | R176F | TTT | Y242T | ACG | I309E | GAG | E375K | AAG |
| P044W | TGG | I110L | CTG | R176G | GGG | Y242V | GTT | I309G | GGT | E375L | CTT |
| P044Y | ACG | I110M | ATG | R176H | CAT | Y242W | TGG | I309H | CAT | E375M | ATG |
| R045A | GCG | I110N | AAT | R176I | ATT | V243A | GCG | I309K | AAG | E375N | AAT |
| R045D | GAT | I110P | CCT | R176K | AAG | V243C | TGT | I309L | CTG | E375P | CCT |
| R045F | TTT | I110R | CGT | R176L | CTT | V243D | GAT | I309M | ATG | E375R | CGT |
| R045G | GGG | I110S | AGT | R176P | CCT | V243F | TTT | I309N | AAT | E375S | TCT |
| R045H | CAT | I110V | GTT | R176Q | CAG | V243G | GGG | I309Q | CAG | E375T | ACT |
| R045I | ATT | I110W | TGG | R176S | AGT | V243H | CAT | I309R | CGT | E375V | GTT |
| R045K | AAG | D111C | TGT | R176T | ACT | V243L | CTT | I309S | AGT | E375Y | TAT |
| R045M | ATG | D111E | GAG | R176V | GTG | V243M | ATG | I309T | ACT | K376A | GCT |
| R045P | CCT | D111G | GGT | R176W | TGG | V243P | CCT | I309V | GTG | K376D | GAT |
| R045Q | CAG | D111H | CAT | P177A | GCT | V243Q | CAG | I309W | TGG | K376E | GAG |
| R045S | TCG | D111I | ATT | P177C | TGT | V243R | AGG | I309Y | TAT | K376G | GGG |
| R045T | ACG | D111K | AAG | P177D | GAT | V243S | AGT | M310A | GCT | K376I | ATT |
| R045V | GTG | D111L | TTG | P177F | TTT | V243T | ACG | M310C | TGT | K376L | TTG |
| R045W | TGG | D111M | ATG | P177G | GGG | V243W | TGG | M310E | GAG | K376M | ATG |
| R045Y | TAT | D111P | ACT | P177H | CAT | V243Y | TAT | M310F | TTT | K376P | CCT |
| I046A | GCG | D111Q | CAG | P177L | CTT | R244A | GCG | M310G | GGG | K376Q | CAG |
| I046C | TGT | D111R | CGG | P177M | ATG | R244D | GAT | M310K | AAG | K376R | CGT |
| I046E | GAG | D111S | AGT | P177Q | CAG | R244G | GGG | M310L | CTG | K376S | AGT |
| I046F | TTT | D111T | ACT | P177R | CGG | R244H | CAT | M310N | AAT | K376T | ACT |
| I046H | CAT | D111V | GTT | P177S | TCT | R244I | ATT | M310P | CCT | K376V | GTG |
| I046L | CTT | D111W | TGG | P177T | ACT | R244K | AAG | M310Q | CAG | K376W | TGG |
| I046M | ATG | D111Y | TAT | P177V | GTT | R244M | ATG | M310R | CGG | K376Y | TAT |
| I046N | AAT | W112C | TGT | P177W | TGG | R244N | AAT | M310S | AGT | G377C | TGT |
| I046P | CCT | W112D | GAT | P177Y | TAT | R244P | CCT | M310V | GTG | G377D | GAT |
| I046R | CGT | W112E | GAG | N178A | GCT | R244Q | CAG | M310W | TGG | G377E | GAG |
| I046S | TCT | W112F | TTT | N178D | GAT | R244S | TCT | M310Y | TAT | G377F | TTT |
| I046T | ACT | W112G | GGG | N178E | GAG | R244T | ACG | R311A | GCT | G377H | CAT |
| I046V | GTT | W112H | CAT | N178G | GGG | R244V | GTG | R311C | TGT | G377I | ATT |
| I046W | TGG | W112I | ATT | N178I | ATT | R244W | TGG | R311E | GAG | G377K | AAG |
| I046Y | TAT | W112L | CTT | N178K | AAG | R244Y | TAT | R311F | TTT | G377L | CTT |
| N047A | GCT | W112N | AAT | N178L | TTG | N245A | GCG | R311G | GGT | G377M | ATG |
| N047D | GAT | W112P | CCT | N178M | ATG | N245C | TGT | R311H | CAT | G377P | CCT |
| N047F | TTT | W112Q | CAG | N178P | CCT | N245F | TTT | R311I | ATT | G377R | AGG |
| N047G | GGG | W112R | CGT | N178R | CGG | N245G | GGG | R311K | AAG | G377S | TCG |
| N047H | CAT | W112S | TCT | N178S | AGT | N245H | CAT | R311L | TTG | G377T | ACT |
| N047I | ATT | W112V | GTT | N178T | ACT | N245I | ATT | R311P | CCT | G377V | GTG |
| N047K | AAG | W112Y | TAT | N178V | GTG | N245K | AAG | R311Q | CAG | G377Y | TAT |
| N047L | CTT | E113A | GCT | N178W | TGG | N245L | CTG | R311S | TCT | G378D | GAT |
| N047M | ATG | E113C | TGT | N178Y | TAT | N245P | CCG | R311T | ACT | G378E | GAG |
| N047P | CCT | E113D | GAT | H179A | GCT | N245Q | CAG | R311V | GTG | G378F | TTT |
| N047Q | CAG | E113F | TTT | H179C | TGT | N245R | CGG | R311W | TGG | G378I | ATT |
| N047R | CGG | E113G | GGG | H179E | GAG | N245S | TCG | S312A | GCT | G378K | AAG |
| N047S | TCT | E113H | CAT | H179G | GGG | N245T | ACG | S312C | TGT | G378L | CTG |
| N047T | ACG | E113L | CTT | H179I | ATT | N245V | GTG | S312E | GAG | G378M | ATG |
| N047V | GTG | E113P | CCT | H179K | AAG | N245W | TGG | S312F | TTT | G378N | AAT |
| N047W | TGG | E113Q | CAG | H179L | CTG | R246A | GCG | S312G | GGG | G378Q | CAG |
| N047Y | TAT | E113R | CGT | H179M | ATG | R246C | TGT | S312H | CAT | G378R | AGG |
| A048C | TGT | E113S | TCT | H179N | AAT | R246D | GAT | S312K | AAG | G378S | TCT |
| A048E | GAG | E113T | ACT | H179P | CCT | R246E | GAG | S312L | CTG | G378T | ACT |
| A048F | TTT | E113V | GTT | H179R | AGG | R246G | GGG | S312M | ATG | G378V | GTG |
| A048G | GGT | E113W | TGG | H179S | AGT | R246H | CAT | S312N | AAT | G378W | TGG |
| A048H | CAT | E113Y | CAT | H179T | ACT | R246I | ATT | S312P | CCT | G378Y | TAT |
| A048I | ATT | E114A | GCT | H179V | GTG | R246K | AAG | S312Q | CAG | K379A | GCT |
| A048K | AAG | E114C | TGT | H179W | TGG | R246L | TTG | S312R | CGG | K379C | TGT |
| A048L | CTG | E114D | GAT | L180A | GCT | R246M | ATG | S312T | ACT | K379E | GAG |
| A048M | ATG | E114G | GGG | L180C | TGT | R246P | CCT | S312V | GTT | K379F | TTT |
| A048N | AAT | E114H | CAT | L180E | GAG | R246S | AGT | S312W | TGG | K379G | GGG |
| A048P | CCT | E114I | ATT | L180F | TTT | R246T | ACG | M313A | GCT | K379H | CAT |
| A048Q | CAG | E114L | CTG | L180G | GGT | R246V | GTT | M313C | TGT | K379I | ATT |
| A048R | CGG | E114M | ATG | L180H | CAT | R246W | TGG | M313D | GAT | K379L | CTT |
| A048S | TCT | E114P | CCT | L180I | ATT | V247A | GCG | M313E | GAG | K379M | ATG |
| A048V | GTT | E114R | CGG | L180K | AAG | V247C | TGT | M313F | TTT | K379N | AAT |
| A048W | TGG | E114S | TCT | L180M | ATG | V247F | TTT | M313G | GGG | K379R | CGT |
| A048Y | TAT | E114T | ACT | L180N | AAT | V247H | CAT | M313H | CAT | K379S | TCT |
| T049A | GCG | E114V | GTG | L180P | CCT | V247I | ATT | M313K | AAG | K379T | ACT |
| T049C | TGT | E114W | TGG | L180R | AGG | V247L | CTG | M313L | CTT | K379V | GTT |
| T049D | GAT | E114Y | TAT | L180S | TCG | V247M | ATG | M313P | CCT | K379W | TGG |
| T049F | TTT | W115A | GCT | L180T | ACT | V247N | AAT | M313R | CGT | F380A | GCT |
| T049G | GGG | W115C | TGT | L180W | TGG | V247P | CCT | M313S | TCG | F380C | TGT |
| T049H | CAT | W115D | GAT | W181A | GCT | V247Q | CAG | M313T | ACT | F380D | GAT |
| T049I | ATT | W115F | TTT | W181C | TGT | V247R | CGT | M313V | GTT | F380E | GAG |
| T049K | AAG | W115G | GGT | W181D | GAT | V247S | TCT | M313Y | TAT | F380G | GGG |
| T049L | TTG | W115H | CAT | W181E | GAG | V247T | ACT | K314A | GCT | F380I | ATT |
| T049N | AAT | W115I | ATT | W181F | TTT | V247W | TGG | K314C | TGT | F380L | CTT |
| T049P | CCG | W115K | AAG | W181H | CAT | V247Y | TAT | K314D | GAT | F380P | CCT |
| T049R | AGG | W115L | CTT | W181I | ATT | R248A | GCT | K314H | CAT | F380Q | CAG |
| T049S | TCG | W115M | ATG | W181K | AAG | R248C | TGT | K314I | ATT | F380R | CGG |
| T049V | GTT | W115P | CCT | W181L | CTG | R248D | GAT | K314L | TTG | F380S | AGT |
| T049W | TGG | W115R | CGG | W181M | ATG | R248E | GAG | K314N | AAT | F380T | ACT |
| G050A | GCG | W115S | AGT | W181N | AAT | R248G | GGG | K314P | CCT | F380V | GTG |
| G050C | TGT | W115V | GTG | W181Q | CAG | R248H | CAT | K314Q | CAG | F380W | TGG |
| G050D | GAT | W115Y | TAT | W181R | CGT | R248I | ATT | K314R | CGG | F380Y | TAT |
| G050E | GAG | R116A | GCT | W181S | TCT | R248L | CTT | K314S | TCG | T381A | AGC |
| G050F | TTT | R116C | TGT | W181V | GTG | R248M | ATG | K314T | ACT | T381E | GAG |
| G050H | CAT | R116D | GAT | G182A | GCT | R248P | CCG | K314V | GTT | T381F | TTT |
| G050L | CTT | R116E | GAG | G182C | TGT | R248S | TCG | K314W | TGG | T381G | GGT |
| G050M | ATG | R116G | GGG | G182D | GAT | R248T | ACG | K314Y | TAT | T381H | CAT |
| G050P | CCT | R116H | CAT | G182E | GAG | R248V | GTG | S315A | GCT | T381K | AAG |
| G050Q | CAG | R116I | ATT | G182H | CAT | R248W | TGG | S315C | TGT | T381L | TTG |
| G050R | CGG | R116L | CTG | G182L | CTT | R248Y | TAT | S315E | GAG | T381N | AAT |
| G050S | AGT | R116N | AAT | G182M | ATG | E249A | GCT | S315G | GGT | T381P | CCT |
| G050V | GTT | R116P | CCT | G182N | AAT | E249G | GGG | S315H | CAT | T381Q | CAG |
| G050W | TGG | R116Q | CAG | G182P | CCT | E249H | CAT | S315I | ATT | T381R | CGT |
| G050Y | TAT | R116S | TCT | G182Q | CAG | E249I | ATT | S315K | AAG | T381S | AGT |
| Q051A | GCG | R116T | ACT | G182R | CGT | E249K | AAG | S315L | CTG | T381V | GTG |
| Q051C | TGT | R116V | GTG | G182S | AGT | E249L | CTG | S315M | ATG | T381W | TGG |
| Q051D | GAT | R116W | TGG | G182T | ACT | E249M | ATG | S315P | CCT | T381Y | TAT |
| Q051F | TTT | P117D | GAT | G182V | GTT | E249P | CCT | S315R | CGG | V382E | GAG |
| Q051H | CAT | P117E | GAG | G182Y | TAT | E249Q | CAG | S315T | ACT | V382G | GGG |
| Q051I | ATT | P117F | TTT | Y183A | GCT | E249R | CGG | S315V | GTT | V382H | CAT |
| Q051K | AAG | P117G | GGT | Y183C | TGT | E249S | TCT | S315W | TGG | V382I | ATT |
| Q051M | ATG | P117H | CAT | Y183D | GAT | E249T | ACT | S315Y | TAT | V382K | AAG |
| Q051N | AAT | P117I | ATT | Y183E | GAG | E249V | GTG | C316A | GCT | V382L | TTG |
| Q051P | CCT | P117K | AAG | Y183G | GGG | E249W | TGG | C316D | GAT | V382M | ATG |
| Q051R | CGG | P117N | AAT | Y183I | ATT | E249Y | TAT | C316E | GAG | V382N | AAT |
| Q051S | TCT | P117Q | CAG | Y183K | AAG | A250C | TGT | C316G | GGG | V382P | CCT |
| Q051T | ACG | P117R | AGG | Y183L | TTG | A250F | TTT | C316I | ATT | V382Q | CAG |
| Q051W | TGG | P117S | TCG | Y183N | AAT | A250G | GGT | C316K | AAG | V382R | CGG |
| Q051Y | TAT | P117T | ACT | Y183P | CCT | A250H | CAT | C316L | CTG | V382S | TCG |
| G052A | GCT | P117V | GTT | Y183Q | CAG | A250K | AAG | C316M | ATG | V382T | ACT |
| G052C | TGT | P117W | TGG | Y183R | CGT | A250L | CTG | C316P | CCT | V382W | TGG |
| G052E | GAG | P117Y | TAT | Y183S | TCT | A250M | ATG | C316R | AGG | V382Y | TAT |
| G052F | TTT | T118C | TGT | Y183V | GTT | A250N | AAT | C316S | TCT | R383A | GCT |
| G052H | CAT | T118D | GAT | Y183W | TGG | A250P | CCT | C316T | ACT | R383E | GAG |
| G052K | AAG | T118E | GAG | Y184A | GCT | A250Q | CAG | C316V | GTT | R383F | TTT |
| G052L | CTT | T118G | GGG | Y184C | TGT | A250R | AGG | C316W | TGG | R383G | GGG |
| G052N | AAT | T118H | CAT | Y184D | GAT | A250S | TCT | C316Y | TAT | R383H | CAT |
| G052P | CCT | T118K | AAG | Y184E | GAG | A250T | ACG | L317A | GCT | R383I | ATT |
| G052Q | CAG | T118L | CTG | Y184F | TTT | A250V | GTG | L317C | TGT | R383K | AAG |
| G052R | CGG | T118M | ATG | Y184G | GGT | A250W | TGG | L317D | GAT | R383L | CTG |
| G052S | AGT | T118N | AAT | Y184H | CAT | I251C | TGT | L317G | GGG | R383M | ATG |
| G052T | ACT | T118P | CCT | Y184K | AAG | I251D | GAT | L317H | CAT | R383N | AAT |
| G052W | TGG | T118Q | CAG | Y184L | CTT | I251F | TTT | L317I | ATT | R383P | CCT |
| G052Y | TAT | T118R | CGT | Y184M | ATG | I251G | GGG | L317K | AAG | R383S | TCG |
| V053A | GCG | T118V | GTT | Y184P | CCT | I251H | CAT | L317M | ATG | R383T | ACT |
| V053C | TGT | T118W | TGG | Y184R | AGG | I251K | AAG | L317N | AAT | R383V | GTG |
| V053D | GAT | T118Y | TAT | Y184S | TCG | I251L | CTT | L317P | CCT | R383W | TGG |
| V053E | GAG | W119A | GCT | Y184V | GTG | I251M | ATG | L317Q | CAG | G384A | GCT |
| V053G | GGG | W119D | GAT | Y184W | TGG | I251P | CCG | L317R | AGG | G384C | TGT |
| V053H | CAT | W119E | GAG | L185A | GCT | I251Q | CAG | L317S | TCG | G384D | GAT |
| V053L | CTG | W119F | TTT | L185D | GAT | I251S | AGT | L317T | ACT | G384E | GAG |
| V053N | AAT | W119G | GGT | L185E | GAG | I251T | ACT | L317W | TGG | G384F | TTT |
| V053P | CCG | W119I | ATT | L185F | TTT | I251V | GTG | L318C | TGT | G384H | CAT |
| V053Q | CAG | W119K | AAG | L185G | GGG | I251W | TGG | L318D | GAT | G384I | ATT |
| V053R | CGG | W119L | CTG | L185I | ATT | I251Y | TAT | L318F | TTT | G384K | AAG |
| V053S | AGT | W119N | AAT | L185K | AAG | R252A | GCT | L318G | GGG | G384L | CTT |
| V053T | ACT | W119P | CCT | L185N | AAT | R252D | GAT | L318H | CAT | G384M | ATG |
| V053W | TGG | W119Q | CAG | L185P | CCT | R252E | GAG | L318I | ATT | G384P | CCT |
| V053Y | TAT | W119R | CGG | L185R | CGG | R252F | TTT | L318K | AAG | G384Q | CAG |
| T054A | GCG | W119S | TCT | L185S | TCG | R252G | GGT | L318M | ATG | G384R | AGG |
| T054D | GAT | W119V | GTT | L185T | ACT | R252H | CAT | L318N | AAT | G384S | TCG |
| T054E | GAG | W119Y | TAT | L185V | GTG | R252I | ATT | L318P | CCT | G384T | ACT |
| T054F | TTT | A120C | TGT | L185W | TGG | R252K | AAG | L318Q | CAG | K385A | GCT |
| T054G | GGG | A120D | GAT | L185Y | TAT | R252L | CTG | L318R | CGG | K385C | TGT |
| T054H | CAT | A120F | TTT | F186A | GCT | R252N | AAT | L318S | AGT | K385G | GGG |
| T054I | ATT | A120G | GGG | F186D | GAT | R252P | CCT | L318T | ACT | K385H | CAT |
| T054M | ATG | A120H | CAT | F186G | GGT | R252S | TCG | L318W | TGG | K385L | CTT |
| T054N | AAT | A120I | ATT | F186H | CAT | R252T | ACT | L319C | TGT | K385M | ATG |
| T054P | CCG | A120L | CTT | F186I | ATT | R252V | GTG | L319E | GAG | K385N | AAT |
| T054Q | CAG | A120N | AAT | F186K | AAG | R252Y | TAT | L319F | TTT | K385P | CCG |
| T054R | CGT | A120P | CCT | F186L | CTT | V253A | GCG | L319G | GGG | K385Q | CAG |
| T054S | AGT | A120R | CGT | F186N | AAT | V253D | GAT | L319H | CAT | K385R | CGT |
| T054V | GTT | A120S | TCT | F186P | CCT | V253E | GAG | L319I | ATT | K385S | TCT |
| T054Y | TAT | A120T | ACT | F186Q | CAG | V253G | GGG | L319K | AAG | K385T | ACG |
| I055A | GCT | A120V | GTG | F186R | AGG | V253H | CAT | L319M | ATG | K385V | GTT |
| I055C | TGT | A120W | TGG | F186S | TCT | V253I | ATT | L319P | CCT | K385W | TGG |
| I055D | GAT | A120Y | TAT | F186V | GTT | V253L | CTG | L319Q | CAG | K385Y | TAT |
| I055F | TTT | R121A | GCT | F186W | TGG | V253M | ATG | L319R | AGG | P386A | GCG |
| I055G | GGG | R121C | TGT | F186Y | TAT | V253N | AAT | L319S | TCG | P386C | TGT |
| I055H | CAT | R121D | GAT | P187A | GCT | V253P | CCT | L319V | GTT | P386F | TTT |
| I055L | CTG | R121E | GAG | P187F | TTT | V253Q | CAG | L319W | TGG | P386G | GGG |
| I055N | AAT | R121F | TTT | P187G | GGG | V253R | CGG | L319Y | TAT | P386H | CAT |
| I055P | CCT | R121G | GGT | P187H | CAT | V253S | TCG | D320C | TGT | P386I | ATT |
| I055Q | CAG | R121H | CAT | P187I | ATT | V253T | ACG | D320E | GAG | P386L | CTT |
| I055R | CGT | R121K | AAG | P187L | CTT | V253W | TGG | D320F | TTT | P386M | ATG |
| I055S | TCG | R121L | CTG | P187M | ATG | S254C | TGT | D320G | GGG | P386N | AAT |
| I055T | ACT | R121M | ATG | P187N | AAT | S254D | GAT | D320H | CAT | P386Q | CAG |
| I055V | GTT | R121P | CCT | P187Q | CAG | S254E | GAG | D320I | ATT | P386R | CGT |
| I055Y | TAT | R121S | TCG | P187R | AGG | S254G | GGG | D320K | AAG | P386S | AGT |
| F056A | GCG | R121T | ACT | P187S | TCG | S254I | ATT | D320L | TTG | P386T | ACG |
| F056C | TGT | R121V | GTT | P187T | ACT | S254K | AAG | D320M | ATG | P386V | GTT |
| F056E | GAG | R121W | TGG | P187V | GTT | S254L | TTG | D320N | AAT | P386Y | TAT |
| F056G | GGG | R121Y | TAT | P187W | TGG | S254N | AAT | D320P | CCT | T387C | TGT |
| F056H | CAT | N122A | GCT | P187Y | TAT | S254P | CCT | D320R | AGG | T387E | GAG |
| F056I | ATT | N122C | TGT | D188A | GCT | S254Q | CAG | D320S | AGT | T387F | TTT |
| F056K | AAG | N122E | GAG | D188C | TGT | S254R | CGG | D320V | GTG | T387G | GGG |
| F056L | TTG | N122F | TTT | D188F | TTT | S254T | ACT | D320W | TGG | T387H | CAT |
| F056N | AAT | N122I | ATT | D188G | GGG | S254V | GTG | D320Y | TAT | T387I | ATT |
| F056P | CCG | N122K | AAG | D188H | CAT | S254W | TGG | N321A | GCT | T387K | AAG |
| F056R | CGT | N122L | CTG | D188L | CTT | S254Y | TAT | N321D | GAT | T387L | CTG |
| F056S | TCT | N122M | ATG | D188M | ATG | K255A | GCG | N321E | GAG | T387M | ATG |
| F056T | ACT | N122P | CCT | D188N | AAT | K255C | TGT | N321G | GGT | T387N | AAT |
| F056V | GTT | N122Q | CAG | D188P | CCT | K255D | GAT | N321H | CAT | T387Q | CAG |
| F056W | TGG | N122R | CGG | D188Q | CAG | K255G | GGT | N321I | ATT | T387S | TCG |
| Y057A | GCT | N122S | TCT | D188R | AGG | K255H | CAT | N321K | AAG | T387V | GTT |
| Y057D | GAT | N122T | ACT | D188S | AGT | K255L | TTG | N321L | CTG | T387W | TGG |
| Y057E | GAG | N122V | GTT | D188T | ACT | K255N | AAT | N321M | ATG | T387Y | TAT |
| Y057F | TTT | N122W | TGG | D188V | GTG | K255P | CCG | N321P | CCT | L388A | GCG |
| Y057G | GGG | W123A | GCT | D188W | TGG | K255Q | CAG | N321R | CGG | L388C | TGT |
| Y057I | ATT | W123C | TGT | C189A | GCT | K255R | CGG | N321S | TCT | L388F | TTT |
| Y057L | TTG | W123D | GAT | C189E | GAG | K255S | TCG | N321T | ACT | L388G | GGG |
| Y057M | ATG | W123E | GAG | C189G | GGT | K255T | ACT | N321V | GTG | L388H | CAT |
| Y057P | CCG | W123G | GGG | C189H | CAT | K255V | GTT | N321Y | TAT | L388I | ATT |
| Y057Q | CAG | W123H | CAT | C189K | AAG | K255W | TGG | Y322C | TGT | L388M | ATG |
| Y057R | CGG | W123L | CTT | C189L | TTG | K255Y | TAT | Y322D | GAT | L388P | CCT |
| Y057S | AGT | W123M | ATG | C189M | ATG | I256A | GCT | Y322E | GAG | L388Q | CAG |
| Y057T | ACG | W123P | CCT | C189N | ACT | I256C | TGT | Y322F | TTT | L388R | CGT |
| Y057V | GTG | W123Q | CAG | C189P | CCT | I256D | GAT | Y322G | GGT | L388S | TCG |
| Y057W | TGG | W123R | AGG | C189R | AGG | I256E | GAG | Y322H | CAT | L388T | ACG |
| V058A | GCT | W123S | AGT | C189S | TCG | I256G | GGG | Y322I | ATT | L388V | GTT |
| V058C | TGT | W123T | ACT | C189T | ACT | I256H | CAT | Y322L | CTG | L388W | TGG |
| V058D | GAT | W123V | GTT | C189V | GTG | I256L | CTT | Y322N | AAT | L388Y | TAT |
| V058G | GGT | W123Y | TAT | C189W | TGG | I256M | ATG | Y322P | CCT | E389A | GCT |
| V058H | CAT | K124A | GCT | C189Y | TAT | I256N | AAT | Y322R | CGT | E389F | TTT |
| V058I | ATT | K124C | TGT | Y190C | TGT | I256P | CCG | Y322S | TCT | E389G | GGT |
| V058K | AAG | K124D | GAT | Y190E | GAG | I256Q | CAG | Y322T | ACT | E389H | CAT |
| V058L | CTT | K124E | GAG | Y190F | TTT | I256R | AGG | Y322V | GTG | E389I | ATT |
| V058N | AAT | K124F | TTT | Y190G | GGG | I256T | ACG | Y322W | TGG | E389K | AAG |
| V058P | CCT | K124G | GGG | Y190H | CAT | I256V | GTT | M323A | GCT | E389L | CTG |
| V058Q | CAG | K124H | CAT | Y190K | AAG | I256W | TGG | M323C | TGT | E389M | ATG |
| V058R | CGG | K124I | ATT | Y190L | CTT | P257A | GCG | M323E | GAG | E389P | CCT |
| V058S | TCG | K124L | CTT | Y190N | AAT | P257C | TGT | M323F | TTT | E389Q | CAG |
| V058W | TGG | K124N | AAT | Y190P | CCT | P257D | GAT | M323G | GGG | E389R | CGG |
| V058Y | TAT | K124P | CCT | Y190Q | CAG | P257G | GGG | M323H | CAT | E389S | TCG |
| D059A | GCT | K124R | CGG | Y190R | CGT | P257I | ATT | M323I | ATT | E389T | ACT |
| D059E | GAG | K124S | TCT | Y190S | TCT | P257K | AAG | M323K | AAG | E389V | GTT |
| D059G | GGG | K124T | ACT | Y190T | ACT | P257L | CTT | M323L | TTG | E389Y | TAT |
| D059H | CAT | K124V | GTG | Y190V | GTG | P257M | ATG | M323N | AAT | D390A | GCG |
| D059I | ATT | K124W | TGG | Y190W | TGG | P257N | AAT | M323P | CCT | D390C | TGT |
| D059L | CTT | P125A | GCT | N191A | GCT | P257Q | CAG | M323R | CGG | D390E | GAG |
| D059M | ATG | P125C | TGT | N191E | GAG | P257R | CGT | M323S | AGT | D390F | TTT |
| D059N | AAT | P125D | GAT | N191F | TTT | P257S | TCG | M323T | ACT | D390G | GGG |
| D059P | CCT | P125G | GGG | N191G | GGG | P257T | ACG | M323V | GTT | D390H | CAT |
| D059Q | CAG | P125H | CAT | N191K | AAG | P257V | GTG | E324A | GCT | D390L | CTT |
| D059R | CGT | P125I | ATT | N191L | TTG | P257W | TGG | E324C | TGT | D390N | AAT |
| D059T | ACG | P125L | CTT | N191M | ATG | D258A | GCG | E324D | GAT | D390P | CCG |
| D059V | GTG | P125N | AAT | N191P | CCT | D258E | GAG | E324F | TTT | D390R | CGG |
| D059W | TGG | P125Q | CAG | N191Q | CAG | D258G | GGG | E324G | GGG | D390S | AGT |
| D059Y | TAT | P125R | CGT | N191R | CGG | D258H | CAT | E324H | CAT | D390T | ACT |
| R060A | GCG | P125S | TCG | N191S | TCG | D258I | ATT | E324L | TTG | D390V | GTG |
| R060D | GAT | P125T | ACT | N191T | ACT | D258L | CTT | E324M | ATG | D390W | TGG |
| R060F | TTT | P125V | GTG | N191V | GTT | D258N | AAT | E324N | AAT | D390Y | TAT |
| R060G | GGT | P125W | TGG | N191W | TGG | D258P | CCG | E324P | CCT | L391A | GCT |
| R060H | CAT | P125Y | TAT | N191Y | TAT | D258Q | CAG | E324R | CGG | L391C | TGT |
| R060I | ATT | K126A | GCT | H192C | TGT | D258R | CGT | E324S | AGT | L391D | GAT |
| R060K | AAG | K126D | GAT | H192F | TTT | D258S | AGT | E324V | GTG | L391G | GGG |
| R060L | CTT | K126E | GAG | H192G | GGT | D258T | ACG | E324W | TGG | L391H | CAT |
| R060N | AAT | K126F | TTT | H192K | AAG | D258V | GTG | E324Y | TAT | L391K | AAG |
| R060P | CCG | K126G | GGT | H192L | CTT | D258W | TGG | T325A | GCT | L391N | AAT |
| R060Q | CAG | K126H | CAT | H192M | ATG | D258Y | TAT | T325C | TGT | L391P | CCT |
| R060S | TCG | K126I | ATT | H192N | AAT | A259E | GAG | T325D | GAT | L391Q | CAG |
| R060T | ACG | K126L | CTG | H192P | CCT | A259G | GGG | T325E | GAG | L391R | CGG |
| R060V | GTT | K126M | ATG | H192Q | CAG | A259I | ATT | T325G | GGT | L391S | TCT |
| R060Y | TAT | K126N | AAT | H192R | CGT | A259K | AAG | T325H | CAT | L391T | ACT |
| L061A | GCT | K126P | CCT | H192S | TCG | A259L | TTG | T325I | ATT | L391V | GTG |
| L061E | GAG | K126Q | CAG | H192T | ACT | A259M | ATG | T325K | AAG | L391W | TGG |
| L061F | TTT | K126R | AGG | H192V | GTT | A259N | AAT | T325M | ATG | L391Y | TAT |
| L061G | GGG | K126S | TCT | H192W | TGG | A259P | CCT | T325N | AAT | E392A | GCT |
| L061H | CAT | K126T | ACT | H192Y | TAT | A259Q | CAG | T325Q | CAG | E392C | TGT |
| L061I | ATT | K126V | GTG | H193A | GCT | A259R | CGT | T325R | CGG | E392F | TTT |
| L061M | ATG | K126W | TGG | H193C | TGT | A259S | AGT | T325S | TCG | E392G | GGG |
| L061N | AAT | K126Y | TAT | H193D | GAT | A259T | ACT | T325V | GTG | E392K | AAG |
| L061P | CCT | D127A | GCT | H193F | TTT | A259V | GTG | T325W | TGG | E392L | CTG |
| L061Q | CAG | D127E | GAG | H193G | GGG | A259W | TGG | I326A | GCT | E392M | ATG |
| L061R | AGG | D127F | TTT | H193K | AAG | A259Y | TAT | I326C | TGT | E392P | CCT |
| L061T | ACT | D127G | GGT | H193L | TTG | K260A | GCG | I326D | GAT | E392Q | CAG |
| L061V | GTT | D127H | CAT | H193M | ATG | K260C | TGT | I326E | GAG | E392R | AGG |
| L061W | TGG | D127K | AAG | H193P | CCG | K260D | GAT | I326G | GGG | E392S | AGT |
| L061Y | TAT | D127L | CTG | H193Q | CAG | K260E | GAG | I326H | CAT | E392T | ACT |
| G062A | GCG | D127M | ATG | H193R | AGG | K260G | GGG | I326K | AAG | E392V | GTT |
| G062C | TGT | D127N | AAT | H193S | TCT | K260H | CAT | I326L | CTT | E392W | TGG |
| G062D | GAT | D127Q | CAG | H193T | ACG | K260L | TTG | I326N | AAT | E392Y | TAT |
| G062F | TTT | D127R | CGT | H193V | GTG | K260M | ATG | I326P | CCT | Q393A | GCG |
| G062I | ATT | D127S | AGT | H193Y | TAT | K260P | CCG | I326R | CGG | Q393C | TGT |
| G062K | AAG | D127T | ACT | Y194A | GCT | K260Q | CAG | I326S | TCT | Q393D | GAT |
| G062L | CTT | D127V | GTT | Y194C | TGT | K260R | CGG | I326V | GTG | Q393F | TTT |
| G062M | ATG | D127W | TGG | Y194E | GAG | K260S | TCT | I326W | TGG | Q393G | GGT |
| G062P | CCT | V128A | GCT | Y194F | TTT | K260V | GTT | I326Y | TAT | Q393H | CAT |
| G062Q | CAG | V128C | TGT | Y194G | GGG | K260W | TGG | L327A | GCT | Q393I | ATT |
| G062R | CGT | V128E | GAG | Y194I | ATT | K260Y | TAT | L327D | GAT | Q393K | AAG |
| G062S | AGT | V128F | TTT | Y194L | TTG | S261A | GCG | L327E | GAG | Q393L | TTG |
| G062T | ACT | V128G | GGG | Y194N | AAT | S261E | GAG | L327F | TTT | Q393M | ATG |
| G062V | GTG | V128H | CAT | Y194P | CCT | S261F | TTT | L327G | GGG | Q393N | AAT |
| G062Y | TAT | V128I | ATT | Y194Q | CAG | S261G | GGG | L327H | CAT | Q393P | CCG |
| Y063A | GCG | V128K | AAG | Y194R | AGG | S261I | ATT | L327M | ATG | Q393R | CGT |
| Y063C | TGT | V128L | CTG | Y194S | TCG | S261K | AAG | L327N | AAT | Q393S | TCG |
| Y063G | GGT | V128P | CCT | Y194T | ACG | S261L | CTT | L327Q | CAG | Q393T | ACG |
| Y063H | CAT | V128Q | CAG | Y194V | GTG | S261M | ATG | L327R | CGG | F394A | GCG |
| Y063I | ATT | V128R | AGG | Y194W | TGG | S261N | AAT | L327S | AGT | F394D | GAT |
| Y063K | AAG | V128S | TCG | K195A | GCG | S261P | CCT | L327T | ACT | F394E | GAG |
| Y063L | CTG | V128W | TGG | K195E | GAG | S261Q | CAG | L327V | GTG | F394G | GGG |
| Y063M | ATG | V128Y | TAT | K195F | TTT | S261R | CGT | L327W | TGG | F394I | ATT |
| Y063N | AAT | Y129A | GCT | K195G | GGT | S261T | ACT | L327Y | TAT | F394K | AAG |
| Y063P | CCT | Y129C | TGT | K195H | CAT | S261V | GTT | N328A | GCT | F394L | CTG |
| Y063R | AGG | Y129D | GAT | K195I | ATT | S261W | TGG | N328C | TGT | F394N | AAT |
| Y063S | TCT | Y129E | GAG | K195L | TTG | P262A | GCG | N328D | GAT | F394P | CCG |
| Y063T | ACG | Y129G | GGG | K195N | AAT | P262D | GAT | N328G | GGT | F394Q | CAG |
| Y063V | GTG | Y129H | CAT | K195Q | CAG | P262E | GAG | N328H | CAT | F394R | CGT |
| Y063W | TGG | Y129L | TTG | K195R | CGT | P262F | TTT | N328I | ATT | F394S | TCG |
| Y064A | GCT | Y129M | ATG | K195S | TCT | P262G | GGG | N328K | AAG | F394T | ACT |
| Y064C | TGT | Y129P | CCT | K195T | ACT | P262H | CAT | N328L | CTT | F394V | GTT |
| Y064D | GAT | Y129Q | CAG | K195V | GTG | P262I | ATT | N328Q | CAG | F394W | TGG |
| Y064E | GAG | Y129R | CGG | K195W | TGG | P262K | AAG | N328R | AGG | S395A | GCG |
| Y064F | TTT | Y129S | AGT | K195Y | TAT | P262Q | CAG | N328S | AGT | S395C | TGT |
| Y064G | GGT | Y129T | ACT | K196A | GCT | P262R | CGT | N328T | ACT | S395D | GAT |
| Y064H | CAT | Y129V | GTT | K196C | TGT | P262S | TCT | N328V | GTG | S395E | GAG |
| Y064I | ATT | Y129W | TGG | K196D | GAT | P262T | ACT | N328W | TGG | S395G | GGG |
| Y064K | AAG | K130C | TGT | K196E | GAG | P262V | GTG | N328Y | TAT | S395H | CAT |
| Y064L | CTT | K130D | GAT | K196G | GGG | P262W | TGG | P329C | TGT | S395K | AAG |
| Y064P | CCT | K130E | GAG | K196I | ATT | P262Y | TAT | P329F | TTT | S395L | CTT |
| Y064Q | CAG | K130G | GGG | K196L | TTG | L263A | GCT | P329G | GGT | S395M | ATG |
| Y064R | CGG | K130H | CAT | K196N | AAT | L263E | GAG | P329H | CAT | S395P | CCT |
| Y064S | AGT | K130I | ATT | K196P | CCG | L263F | TTT | P329I | ATT | S395R | CGG |
| Y064T | ACT | K130L | TTG | K196R | CGT | L263G | GGG | P329K | AAG | S395T | ACG |
| Y064V | GTT | K130N | AAT | K196S | TCG | L263H | CAT | P329L | CTG | S395V | GTT |
| Y064W | TGG | K130Q | CAG | K196T | ACT | L263K | AAG | P329N | AAT | S395W | TGG |
| P065A | GCT | K130R | AGG | K196V | GTG | L263M | ATG | P329Q | CAG | S395Y | TAT |
| P065C | TGT | K130S | TCT | K196W | TGG | L263N | AAT | P329R | CGT | E396A | GCG |
| P065D | GAT | K130T | ACT | K196Y | TAT | L263P | CCG | P329S | AGT | E396C | TGT |
| P065F | TTT | K130V | GTG | P197A | GCT | L263Q | CAG | P329T | ACT | E396D | GAT |
| P065G | GGG | K130W | TGG | P197C | TGT | L263R | CGG | P329V | GTT | E396F | TTT |
| P065H | CAT | K130Y | TAT | P197D | GAT | L263S | AGT | P329W | TGG | E396G | GGG |
| P065I | ATT | N131C | TGT | P197E | GAG | L263T | ACT | P329Y | TAT | E396H | CAT |
| P065K | AAG | N131E | GAG | P197F | TTT | L263V | GTT | Y330A | GCT | E396I | ATT |
| P065N | AAT | N131F | TTT | P197G | GGT | L263W | TGG | Y330C | TGT | E396L | CTT |
| P065R | CGG | N131G | GGG | P197H | CAT | P264A | GCG | Y330D | GAT | E396P | CCG |
| P065S | TCG | N131H | CAT | P197K | AAG | P264D | GAT | Y330E | GAG | E396Q | CAG |
| P065T | ACG | N131I | ATT | P197L | TTG | P264E | GAG | Y330F | TTT | E396R | AGG |
| P065V | GTT | N131L | CTT | P197M | ATG | P264F | TTT | Y330G | GGT | E396S | TCT |
| P065W | TGG | N131M | ATG | P197Q | CAG | P264G | GGT | Y330I | ATT | E396T | ACT |
| P065Y | TAT | N131P | CCT | P197R | CGT | P264H | CAT | Y330L | CTG | E396V | GTG |
| Y066A | GCG | N131Q | CAG | P197S | AGT | P264L | CTT | Y330M | ATG | E396Y | TAT |
| Y066C | TGT | N131R | CGG | P197T | ACT | P264M | ATG | Y330N | AAT | K397A | GCT |
| Y066D | GAT | N131S | AGT | P197W | TGG | P264N | AAT | Y330P | CCT | K397C | TGT |
| Y066E | GAG | N131T | ACT | G198A | GCT | P264R | CGG | Y330R | AGG | K397E | GAG |
| Y066G | GGT | N131V | GTG | G198C | TGT | P264S | AGT | Y330S | AGT | K397F | TTT |
| Y066H | CAT | N131Y | TAT | G198D | GAT | P264T | ACT | Y330V | GTT | K397G | GGT |
| Y066I | ATT | R132A | GCT | G198E | GAG | P264V | GTT | I331V | GTG | K397I | ATT |
| Y066K | AAG | R132C | TGT | G198H | CAT | P264W | TGG | Y330W | TGG | K397L | TTG |
| Y066L | CTG | R132E | GAG | G198L | CTG | P264Y | TAT | I331A | GCT | K397M | ATG |
| Y066N | AAT | R132F | TTT | G198N | AAT | V265A | GCG | I331C | TGT | K397N | AAT |
| Y066P | CCT | R132H | CAT | G198P | CCG | V265C | TGT | I331D | GAT | K397P | CCG |
| Y066R | CGG | R132I | ATT | G198Q | CAG | V265D | GAT | I331E | GAG | K397Q | CAG |
| K397T | ACT | R132K | AAG | G198R | AGG | V265E | GAG | I331F | TTT | K397R | AGG |
| K397V | GTT | R132L | TTG | G198S | TCT | V265F | TTT | I331H | CAT | K397S | TCG |
| F398A | GCT | L406P | CCT | K415G | GGT | C423T | ACT | A432L | TTG | E441D | GAT |
| F398C | TGT | L406Q | CAG | K415L | CTG | C423V | GTG | A432M | ATG | E441F | TTT |
| F398E | GAG | L406R | CGG | K415M | ATG | C423W | TGG | A432N | AAT | E441G | GGG |
| F398G | GGT | L406S | AGT | K415P | CCG | I424A | GCT | A432P | CCT | E441H | CAT |
| F398H | CAT | L406T | ACG | K415Q | CAG | I424C | TGT | A432R | AGG | E441K | AAG |
| F398I | ATT | L406V | GTT | K415R | CGG | I424E | GAG | A432S | TCT | E441L | CTT |
| F398L | CTT | L406Y | TAT | K415S | TCT | I424G | GGG | A432V | GTG | E441N | AAT |
| F398N | AAT | S407A | GCG | K415T | ACT | I424H | CAT | A432Y | TAT | E441Q | CAG |
| F398P | CCT | S407D | GAT | K415V | GTG | I424K | AAG | F433A | GCT | E441R | CGG |
| F398R | AGG | S407E | GAG | K415W | TGG | I424L | CTT | F433C | TGT | E441S | AGT |
| F398S | TCT | S407F | TTT | K415Y | TAT | I424N | AAT | F433D | GAT | E441T | ACT |
| F398T | ACT | S407G | GGT | D416C | TGT | I424Q | CAG | F433E | GAG | E441V | GTG |
| F398V | GTT | S407H | CAT | D416F | TTT | I424R | CGG | F433G | GGG | E441Y | TAT |
| F398W | TGG | S407L | CTG | D416G | GGT | I424S | TCG | F433H | CAT | E442C | TGT |
| F398Y | TAT | S407M | ATG | D416H | CAT | I424T | ACT | F433I | ATT | E442G | GGG |
| Y399A | GCG | S407N | AAT | D416I | ATT | I424V | GTT | F433K | AAG | E442H | CAT |
| Y399C | TGT | S407P | CCT | D416K | AAG | I424W | TGG | F433L | TTG | E442K | AAG |
| Y399D | GAT | S407Q | CAG | D416L | CTT | I424Y | TAT | F433P | CCT | E442L | CTT |
| Y399E | GAG | S407R | CGG | D416N | AAT | A425C | TGT | F433R | CGG | E442M | ATG |
| Y399G | GGG | S407T | ACG | D416Q | CAG | A425D | GAT | F433S | AGT | E442N | AAT |
| Y399K | AAG | S407V | GTG | D416R | CGG | A425E | GAG | F433T | ACT | E442P | CCT |
| Y399M | ATG | S407W | TGG | D416S | TCT | A425G | GGT | F433V | GTG | E442Q | CAG |
| Y399N | AAT | C408A | GCG | D416T | ACG | A425I | ATT | F433W | TGG | E442R | CGG |
| Y399P | CCT | C408E | GAG | D416V | GTG | A425K | AAG | L434F | TTT | E442S | AGT |
| Y399Q | CAG | C408F | TTT | D416W | TGG | A425L | TTG | L434G | GGT | E442T | ACT |
| Y399R | CGG | C408G | GGG | D416Y | TAT | A425M | ATG | L434H | CAT | E442V | GTG |
| Y399S | TCG | C408I | ATT | T417A | GCT | A425N | AAT | L434I | ATT | E442W | TGG |
| Y399T | ACG | C408K | AAG | T417D | GAT | A425P | CCT | L434K | AAG | E442Y | TAT |
| Y399V | GTT | C408L | CTT | T417E | GAG | A425R | AGG | L434M | ATG | P443A | GCT |
| Y399W | TGG | C408N | AAT | T417F | TTT | A425S | AGT | L434N | AAT | P443D | GAT |
| C400A | GCG | C408P | CCT | T417G | GGG | A425V | GTG | L434P | CCT | P443E | GAG |
| C400D | GAT | C408R | CGT | T417H | CAT | A425W | TGG | L434Q | CAG | P443F | TTT |
| C400E | GAG | C408S | TCG | T417I | ATT | A425Y | TAT | L434R | CGG | P443G | GGG |
| C400F | TTT | C408T | ACT | T417K | AAG | D426A | GCT | L434S | AGT | P443H | CAT |
| C400G | GGG | C408V | GTT | T417L | TTG | D426C | TGT | L434T | ACT | P443I | ATT |
| C400I | ATT | C408W | TGG | T417M | ATG | D426E | GAG | L434V | GTT | P443L | CTT |
| C400L | CTG | C408Y | TAT | T417P | CCT | D426F | TTT | L434W | TGG | P443M | ATG |
| C400M | ATG | K409A | GCG | T417Q | CAG | D426G | GGG | L434Y | TAT | P443N | AAT |
| C400P | CCG | K409C | TGT | T417R | CGT | D426I | ATT | K435A | GCT | P443Q | CAG |
| C400Q | CAG | K409D | GAT | T417S | TCG | D426K | AAG | K435C | TGT | P443R | AGG |
| C400R | CGG | K409E | GAG | T417W | TGG | D426L | CTG | K435E | GAG | P443S | TCT |
| C400S | AGT | K409G | GGT | D418A | GCT | D426M | ATG | K435F | TTT | P443T | ACT |
| C400T | ACG | K409H | CAT | D418C | TGT | D426N | AAT | K435G | GGT | P443W | TGG |
| C400V | GTG | K409I | ATT | D418E | GAG | D426P | CCT | K435H | CAT | Q444C | TGT |
| C400Y | TAT | K409L | CTG | D418F | TTT | D426Q | CAG | K435I | ATT | Q444D | GAT |
| S401A | GCT | K409P | CCG | D418G | GGT | D426R | CGT | K435L | CTG | Q444E | GAG |
| S401C | TGT | K409Q | CAG | D418I | ATT | D426S | TCG | K435P | CCT | Q444F | TTT |
| S401D | GAT | K409R | AGG | D418L | TTG | D426Y | TAT | K435R | AGG | Q444G | GGG |
| S401E | GAG | K409S | TCG | D418M | ATG | G427A | GCT | K435S | TCT | Q444H | CAT |
| S401F | TTT | K409T | ACG | D418N | AAT | G427C | TGT | K435T | ACT | Q444I | ATT |
| S401G | GGG | K409V | GTG | D418P | CCT | G427F | TTT | K435V | GTT | Q444K | AAG |
| S401H | CAT | K409W | TGG | D418Q | CAG | G427H | CAT | K435W | TGG | Q444L | CTG |
| S401K | AAG | A412Y | TAT | D418R | CGG | G427I | ATT | K435Y | TAT | Q444M | ATG |
| S401L | CTT | E410D | GAT | D418S | TCG | G427K | AAG | P436C | TGT | Q444N | AAT |
| S401N | AAT | E410G | GGG | D418V | GTG | G427L | CTG | P436D | GAT | Q444R | CGG |
| S401Q | CAG | E410I | ATT | D418Y | TAT | G427P | CCT | P436E | GAG | Q444V | GTT |
| S401R | CGT | E410K | AAG | A419D | GAT | G427Q | CAG | P436G | GGG | Q444W | TGG |
| S401T | ACT | E410L | CTT | A419E | GAG | G427R | CGT | P436H | CAT | Q444Y | TAT |
| S401W | TGG | E410M | ATG | A419F | TTT | G427S | AGT | P436I | ATT | I445A | GCT |
| S401Y | TAT | E410N | AAT | A419G | GGG | G427T | ACT | P436K | AAG | I445C | TGT |
| C402A | GCT | E410P | CCG | A419H | CAT | G427V | GTG | P436L | CTG | I445D | GAT |
| C402D | GAT | E410Q | CAG | A419I | ATT | G427W | TGG | P436M | ATG | I445G | GGG |
| C402E | GAG | E410R | CGT | A419K | AAG | G427Y | TAT | P436Q | CAG | I445H | CAT |
| C402F | TTT | E410S | TCG | A419L | CTT | V428A | GCT | P436R | CGG | I445K | AAG |
| C402G | GGG | E410T | ACG | A419N | AAT | V428C | TGT | P436S | TCT | I445L | CTT |
| C402L | TTG | E410V | GTG | A419P | CCT | V428D | GAT | P436T | ACT | I445M | ATG |
| C402M | ATG | E410W | TGG | A419R | CGG | V428E | GAG | P436W | TGG | I445N | AAT |
| C402P | CCT | E410Y | TAT | A419S | TCT | V428F | TTT | P436Y | TAT | I445P | CCT |
| C402Q | CAG | K411A | GCT | A419T | ACT | V428G | GGT | P437A | GCT | I445Q | CAG |
| C402R | CGG | K411D | GAT | A419W | TGG | V428H | CAT | P437D | GAT | I445R | AGG |
| C402S | TCT | K411E | GAG | A419Y | TAT | V428L | CTT | P437F | TTT | I445S | AGT |
| C402T | ACG | K411F | TTT | V420A | GCT | V428M | ATG | P437G | GGT | I445T | ACT |
| C402V | GTT | K411G | GGG | V420D | GAT | V428N | AAT | P437H | CAT | I445V | GTG |
| C402W | TGG | K411H | CAT | V420F | TTT | V428P | CCT | P437I | ATT | I445W | TGG |
| C402Y | TAT | K411I | ATT | V420G | GGT | V428R | CGG | P437K | AAG | I445Y | TAT |
| Y403A | GCT | K411L | CTG | V420H | CAT | V428S | TCG | P437L | CTG | F446A | GCT |
| Y403C | TGT | K411N | AAT | V420I | ATT | V428T | ACT | P437M | ATG | F446C | TGT |
| Y403E | GAG | K411P | CCT | V420K | AAG | V428Y | TAT | P437Q | CAG | F446D | GAT |
| Y403F | TTT | K411R | AGG | V420L | CTT | C429A | GCT | P437R | CGT | F446E | GAG |
| Y403G | GGT | K411S | TCG | V420N | AAT | C429D | GAT | P437S | TCT | F446G | GGG |
| Y403H | CAT | K411T | ACT | V420P | CCT | C429G | GGT | P437T | ACT | F446H | CAT |
| Y403K | AAG | K411V | GTT | V420R | AGG | C429I | ATT | P437W | TGG | F446I | ATT |
| Y403L | TTG | K411W | TGG | V420S | TCT | C429K | AAG | P437Y | TAT | F446K | AAG |
| Y403M | ATG | A412D | GAT | V420T | ACT | C429L | TTG | M438A | GCT | F446L | TTG |
| Y403N | AAT | A412E | GAG | V420W | TGG | C429M | ATG | M438C | TGT | F446M | ATG |
| Y403P | CCG | A412G | GGG | V420Y | TAT | C429N | AAT | M438D | GAT | F446Q | CAG |
| Y403Q | CAG | A412H | CAT | D421A | GCT | C429P | CCT | M438E | GAG | F446R | CGG |
| Y403R | CGG | A412I | ATT | D421E | GAG | C429R | CGG | M438G | GGG | F446T | ACT |
| Y403S | TCT | A412L | CTG | D421G | GGT | C429S | TCG | M438L | TTG | F446V | GTT |
| Y403T | ACG | A412N | AAT | D421H | CAT | C429T | ACT | M438N | AAT | F446W | TGG |
| S404A | GCT | A412P | CCT | D421I | ATT | C429V | GTT | M438P | CCT | Y447D | GAT |
| S404C | TGT | A412Q | CAG | D421K | AAG | C429W | TGG | M438Q | CAG | Y447E | GAG |
| S404D | GAT | A412R | CGG | D421L | TTG | C429Y | TAT | M438R | AGG | Y447F | TTT |
| S404F | TTT | A412S | AGT | D421M | ATG | I430A | GCT | M438S | TCG | Y447G | GGT |
| S404G | GGT | A412V | GTT | D421N | AAT | I430D | GAT | M438T | ACT | Y447I | ATT |
| S404H | CAT | A412W | TGG | D421Q | CAG | I430E | GAG | M438V | GTG | Y447K | AAG |
| S404L | CTT | D413A | GCG | D421R | CGG | I430G | GGG | M438W | TGG | Y447L | CTT |
| S404M | ATG | D413E | GAG | D421S | TCG | I430H | CAT | M438Y | TAT | Y447M | ATG |
| S404N | AAT | D413F | TTT | D421T | ACT | I430K | AAG | E439A | GCT | Y447N | AAT |
| S404P | CCT | D413G | GGT | D421W | TGG | I430L | TTG | E439C | TGT | Y447P | CCT |
| S404R | AGG | D413H | CAT | D421Y | TAT | I430M | ATG | E439F | TTT | Y447Q | CAG |
| S404T | ACG | D413I | ATT | V422A | GCT | I430N | AAT | E439G | GGG | Y447R | AGG |
| S404V | GTG | D413K | AAG | V422C | TGT | I430P | CCT | E439H | CAT | Y447T | ACT |
| S404W | TGG | D413L | CTG | V422D | GAT | I430R | AGG | E439K | AAG | Y447V | GTT |
| S404Y | TAT | D413N | AAT | V422E | GAG | I430S | TCT | E439L | CTT | Y447W | TGG |
| T405A | GCG | D413P | CCG | V422G | GGG | I430T | ACT | E439N | AAT | | |
| T405C | TGT | D413Q | CAG | V422H | CAT | I430V | GTT | E439P | CCT | | |
| T405F | TTT | D413R | CGT | V422I | ATT | I430W | TGG | E439Q | CAG | | |
| T405G | GGG | D413S | TCG | V422L | CTG | D431A | GCT | E439R | CGG | | |
| T405I | ATT | D413T | ACT | V422M | ATG | D431E | GAG | E439S | TCG | | |
| T405K | AAG | D413W | TGG | V422N | AAT | D431G | GGT | E439T | ACT | | |
| T405L | TTG | V414A | GCG | V422P | CCT | D431H | CAT | E439V | GTT | | |
| T405M | ATG | V414D | GAT | V422Q | CAG | D431I | ATT | E439W | TGG | | |
| T405P | CCG | V414E | GAG | V422R | CGT | D431K | AAG | T440A | GCT | | |
| T405Q | CAG | V414F | TTT | V422S | TCG | D431L | CTT | T440D | GAT | | |
| T405R | CGT | V414G | GGT | V422T | ACT | D431N | AAT | T440E | GAG | | |
| T405S | TCT | V414H | CAT | V422W | TGG | D431P | CCT | T440F | TTT | | |
| T405V | GTG | V414I | ATT | V422Y | TAT | D431Q | CAG | T440G | GGG | | |
| T405W | TGG | V414K | AAG | C423A | GCT | D431R | CGT | T440H | CAT | | |
| T405Y | TAT | V414L | TTG | C423D | GAT | D431S | TCT | T440I | ATT | | |
| L406A | GCT | V414M | ATG | C423E | GAG | D431V | GTT | T440L | CTT | | |
| L406C | TGT | V414Q | CAG | C423F | TTT | D431W | TGG | T440M | ATG | | |
| L406D | GAT | V414R | AGG | C423G | GGG | D431Y | TAT | T440P | CCT | | |
| L406E | GAG | V414S | TCG | C423H | CAT | A432C | TGT | T440Q | CAG | | |
| L406F | TTT | V414T | ACT | C423L | CTG | A432E | GAG | T440R | AGG | | |
| L406G | GGT | V414Y | TAT | C423M | ATG | A432F | TTT | T440S | AGT | | |
| L406I | ATT | K415A | GCG | C423P | CCT | A432G | GGG | T440V | GTG | | |
| L406N | AAT | K415C | TGT | C423Q | CAG | A432H | CAT | T440Y | TAT | | |
| | | K415D | GAT | C423R | AGG | A432I | ATT | E441A | GCT | | |
| | | K415E | GAG | C423S | TCG | A432K | AAG | E441C | TGT | | |

2. Expression

For expression of each mutant, HZ24-PH20-IRES-SEAP plasmid DNA containing cDNA encoding one of the variant PH20 or encoding wildtype PH20 was transfected into monolayer CHO-S cells (Invitrogen, Cat. No. 11619-012) using Lipofectamine 2000 (Invitrogen, Cat. No. 11668-027) according to the protocol suggested by the manufacturer. CHO-S cells were seeded the night before transfection and grown in DMEM with 10% FBS to be 80% confluent the next day. Then, the medium of the CHO-S cells was replaced with Opti-MEM. A mixture of plasmid DNA and lipofectamine was made (0.2 μg DNA and 0.5 μL Lipofetamine). The Lipofectamine/DNA mixture was added to CHO-S cells and incubated overnight. The next day, the cells were supplemented with CD-CHO serum free medium (Invitrogen, Cat. No. 10743-029). Supernatant from transfected cells was collected at various time points after transfection, and generally 96 hours after transfection. The supernatant, containing the variant PH20 protein or wildtype PH20 having a sequence of amino acids set forth in SEQ ID NO:3, was stored at −20° C. Activities of the supernatants were screened as described in the following examples.

### Example 3

### Screening Assay to Assess Hyaluronidase Activity of PH20 Variants

1. Generation of Biotinylated HA (bHA) Substrate

A 1.2-MDa HA (Lifecore) was biotinylated for use as a substrate in the hyaluronidase activity assay. First, 1.2 grams (g) of 1.2 MDa HA was dissolved at 4° C. in 600 mL ddH₂0 for a week at a concentration of 2 mg/mL with stirring. Next, 645.71 mg Biotin Hydrazide was dissolved in 100 mL DMSO to a concentration of 25 mM (6.458 mg/mL, 247.8 mg in 38.37 mL DMSO). The biotin solution was warmed briefly at 37° C. until the solution was clear. Also, 368.61 mg Sulfo-NHS in 20 mL ddH₂O was dissolved to make a 100× solution (18.4 mg/mL Sulfo-NHS). A 30 mM (1000×) water-soluble carbodiimide EDC solution was made by dissolving 17.63 mg EDC in 3 mL ddH20 at a concentration of 5.7513 mg/mL right before the reaction was started.

To four (4) 1000-mL sterile capped bottles, the following components were added at room temperature (RT) and in the following order with stirring: 1) 200 mL of 2 mg/mL HA solution; 2) 80 mL of 0.5M MES, pH 5.0 with gentle mixing; and 3) 91.6 mL of ddH₂O with gentle mixing. Next, 24 mL of 25 mM Biotin-Hydrazide and 4 mL of 100× Sulfo-NHS solution were added sequentially, immediately followed by the addition of 500 μL EDC. After the addition of each component, the solution was mixed by inverting three times and stirring. After the addition of the last component, the solution was mixed by stirring overnight at 4° C. Then, Guanidine hydrochloride was added to a final concentration of 4 M by adding 38.2 g per 100 mL and was allowed to dissolve completely before adjusting the solution volume to 600 mL with ddH₂O.

For dialysis, 200 mL from each batch of the conjugated HA guanidine hydrochloride solution was transferred into dialysis membranes. Over the course of three days, the solution was dialyzed against ddH₂O with a change in ddH₂O at least six times. The resulting volume of about 840 mL was adjusted to a final volume of 1000 mL with ddH₂O. The final concentration of the biotinylated hyaluronan (bHA) was 0.4 mg/mL.

2. Hyaluronidase Activity Assay

The enzyme assay was a modification of the method described by Frost et al. (1997) (A Microtiter-Based Assay for Hyaluronidase Activity Not Requiring Specialized Reagents. Analytical Biochemistry (1997) 251:263-269) that provides a measure of PH20 hyaluronidase activity.

First, biotinylated HA (bHA) substrate was bound to plastic microtiter plates to generate assay plates. Briefly, 100 μl of b-HA at 1 mg/mL in 0.5 M carbonate buffer (pH 9.6) was dispensed into each well of a high bind microplate (Immunolon 4 HBX extra high binding; Thermo Scientific). The plate was covered with a plate sealer and stored between 2-8° C. for 24-48 hours.

Then, the assay plate was washed with 1× phosphate buffered saline (PBS) wash buffer containing 0.05% (v/v) Tween 20 (PBST). PBST was generated from 1×PBS (generated from Catalog No. P5368, Sigma (10 mM Phosphate Buffer, 2.7 mM Potassium Chloride, 137 mM Sodium Chloride, pH 7.4) by placing the contents of one packet of PBS into a 1-L graduated cylinder with 800 mL deionized water, dissolved by stirring or shaking and adding sufficient quantity of water to 1 L) by adding 500 μl Tween 20 (Catalog No. 6505; EMD Bioscience) to 900 mL of 1×PBS and adding sufficient quantity of water to 1 L. Washing was done using the BioTek ELx405 Select CW plate washer (BioTek) by washing five (5) times with 300 μl PBST wash buffer per well for each wash. At the end of each wash, the plate was tapped on a paper towel to remove excess liquid from each well. Prior to incubation with samples, 200 μl Blocking Buffer (1.0% w/v Bovine Serum Albumin (BSA) in PBS) was added to each well and the assay plate was incubated at 37° C. for approximately 1 hour prior. The Blocking buffer was generated by adding 2.5 g of BSA (Catalog No. 001-000-162; Jackson Immuno Research) to 200 mL 1×PBS, stirring, adding a sufficient quantity of 1×PBS to 250 mL and filtering through an 0.2 μM PES filter unit.

Transfected variant or wildtype PH20 supernatants generated as described in Example 1 were diluted in duplicate 1:25 in assay diluent buffer (pH 7.4 HEPES buffer; 10 mM HEPES, 50 mM NaCl, 1 mM CaCl₂, 1 mg/mL BSA, pH 7.4, 0.05% Tween-20) in uncoated 4XHB high bound microplates. For the standard curve, 1:3 serial dilutions of rHuPH20 (generated as described in Example 1 with a specific activity of 145 U/mL) were made in assay diluent buffer in duplicate starting from 3 U/mL for standards as follows: 3 U/mL, 1 U/mL, ⅓ U/mL, 1/9 U/mL, 1/27 U/mL, 1/81 U/mL, and 1/243 U/mL. One hundred microliters (100 μl) of each standard and sample were transferred to the assay plates and incubated for approximately 1.5 hours at 37° C.

After the incubation, the plate was washed with PBST using the BioTek ELx405 Select CW plate washer by washing five (5) times with 300 μl PBST wash buffer per well for each wash. At the end of each wash, the plate was tapped on a paper towel to remove excess liquid from each well. Then, 100 μl of 1:5000 diluted Streptavidin-HRP (SA-HRP) was added to each well of the plate and incubated at ambient temperature for approximately 1 hour. For the dilution, a 1 mg/mL stock of Streptavidin-HRP conjugate (Catalog No. 21126; Thermo Scientific) was diluted 1:5000 into dilution buffer (1 mg/mL BSA, 0.025% Tween20, 137 mM NaCl, 20 mM Tris pH 7.5). After the incubation, the plate was washed with PBST using the BioTek ELx405 Select CW plate washer by washing five (5) times with 300 μl PBST wash buffer per well for each wash. At the end of each wash, the plate was tapped on a paper towel to remove excess liquid from each well. Then, 100 μl of TMB solution (Catalog No. 52-00-03, KPL; ambient temperature and protected from light) was added to each well for approximately five (5) minutes at room temperature or until an optimal color development was yielded. To stop the reaction, 100 μl 1.0 N Sulfuric Acid or TMB Stop solution (Catalog No. 50-85-06) were added to each well and the plates tapped to mix. Optical density was measured at 450 nm within 30 minutes of adding the stop solution. Since more PH20 in a standard or sample would lead to less bHA available to bind SA-HRP, the optical density (450 nm) value was inversely proportional to the concentration of hyaluronidase activity in each specimen.

3. SEAP Activity

Activity of secreted alkaline phosphatase (SEAP) in the cell culture supernatant also was measured using a colorimetric assay of placental alkaline phosphatase using pNPP as a phosphatase substrate (Anaspec SensoLyte pNPP SEAP kit; Catalog No. 72144, Anaspec) according to the manufacturer's instructions. The absorbance signal was measured at optical density (OD) of 405 nm

The criteria for the high throughput (HTP) screening were that the transfected supernatant resulted in a SEAP signal of ≧0.1 and the signal for the rHuPH20 wildtype control produced a signal of ≧1 U/mL. Also, the criteria for each screen were that the standard curves had a signal to noise ratio (S/N) for the 0 U/mL standard versus the 3 u/mL standard at OD₄₀₅ of ≧5, had less than three (3) standards with a coefficient of variation (CV) ≧10%, and at least four (4) of the standards were in the linear range.

### Example 4

### PH20 Variants with Altered Hyaluronidase Activity

Each generated variant was screened for hyaluonidase activity as described in Example 3. The secreted alkaline phosphatase (SEAP) expression was also measured and used to normalize PH20 activity of each variant to the PH20 wildtype. Mutants that exhibited altered hyaluronidase activity compared to wildtype were identified.

1. Active Mutants

Active mutants in which at least one duplicate sample exhibited greater than 40% of wildtype activity when normalized to SEAP activity were selected. The identified active mutants are set forth in Table 7. In the Table, the amino acid replacement compared to the sequence of amino acids of PH20 set forth in SEQ ID NO:3 is indictated. The Table sets forth the average hyaluronidase activity of tested duplicates normalized by SEAP values compared to average of wildtype PH20 activities in each plate, which also were normalized by their own SEAP values. For example, a value of 0.40 indicates that the variant exhibits 40% of the hyaluronidase activity of wildtype PH20, a value of 1 indicates that the variant exhibits a similar hyaluronidase activity of wildtype and a value of 3.00 indicates that the variant exhibits 300% of the hyaluronidase activity of wildtype PH20 or 3-fold increased activity compared to wildtype.

TABLE 7
ACTIVE MUTANTS

| | AvgNorm |
|---|---|
| mutant | Act. |
| L001A | 0.95 |
| L001C | 0.89 |
| L001E | 0.55 |
| L001F | 0.41 |
| L001G | 0.62 |
| L001H | 1.90 |
| L001K | 1.39 |
| L001N | 0.87 |
| L001P | 0.92 |
| L001Q | 3.27 |
| L001R | 0.72 |
| L001S | 0.74 |
| L001T | 0.99 |
| L001V | 1.00 |
| L001W | 0.88 |
| N002A | 0.61 |
| N002C | 0.4 |
| G291C | 0.27 |
| N002G | 0.44 |
| N002L | 0.46 |
| N002P | 0.54 |
| N002Q | 0.84 |
| N002S | 0.78 |
| N002T | 1.05 |
| N002V | 0.65 |
| F003E | 0.42 |
| F003H | 0.68 |
| F003L | 0.59 |
| F003Y | 0.50 |
| R004A | 0.73 |
| R004I | 0.54 |
| R004S | 0.60 |
| R004T | 0.66 |
| R004V | 1.09 |
| A005H | 0.44 |
| P006A | 0.78 |
| P006H | 0.58 |
| P006K | 0.80 |
| P006L | 0.76 |
| P006N | 0.40 |
| P006Q | 0.89 |
| P006R | 0.56 |
| P007M | 0.57 |
| V008I | 1.17 |
| V008L | 0.53 |
| V008M | 0.47 |
| V008P | 0.33 |
| I009K | 0.69 |
| I009L | 1.08 |
| I009R | 0.53 |
| I009S | 0.98 |
| I009V | 0.84 |
| P010D | 0.62 |
| P010E | 0.66 |
| P010G | 0.55 |
| P010H | 0.43 |
| P010N | 0.55 |
| P010Q | 0.89 |
| P010R | 0.73 |
| P010S | 0.55 |
| P010W | 0.59 |
| N011D | 0.54 |
| N011G | 0.45 |
| N011H | 0.69 |
| N011K | 0.58 |
| N011S | 0.39 |
| M310F | 0.30 |
| V012A | 0.56 |
| V012E | 1.86 |
| V012I | 0.68 |
| V012K | 0.65 |
| V012L | 0.44 |
| V012N | 0.46 |
| V012R | 0.50 |
| V012S | 0.75 |
| V012T | 1.50 |
| P013H | 0.46 |
| P013S | 0.68 |
| P013T | 0.90 |
| P013Y | 0.51 |
| F014D | 0.64 |
| F014I | 0.42 |
| F014M | 0.47 |
| F014V | 0.46 |
| L015A | 0.65 |
| L015M | 0.45 |
| L015V | 2.20 |
| A020S | 0.50 |
| S022H | 0.57 |
| S022M | 0.49 |
| S022T | 0.48 |
| S022Y | 0.45 |
| E023D | 0.97 |
| F024A | 0.69 |
| F024E | 3.99 |
| F024G | 0.75 |
| F024H | 2.07 |
| F024I | 0.70 |
| F024K | 0.96 |
| F024L | 0.62 |
| F024M | 0.85 |
| F024N | 0.60 |
| F024R | 1.22 |
| F024T | 1.18 |
| F024V | 1.15 |
| F024Y | 0.90 |
| L026A | 1.30 |
| L026E | 3.22 |
| L026G | 0.81 |
| L026H | 0.97 |
| L026I | 0.51 |
| L026K | 1.88 |
| L026M | 1.43 |
| L026P | 0.55 |
| L026Q | 1.44 |
| L026R | 1.43 |
| L026S | 0.78 |
| L026T | 0.87 |
| L026V | 0.52 |
| L026W | 0.53 |
| L026Y | 0.52 |
| G027A | 0.79 |
| G027D | 1.22 |
| G027E | 1.18 |
| G027F | 0.61 |
| G027H | 1.11 |
| G027I | 0.41 |
| G027K | 2.71 |
| G027L | 0.76 |
| G027P | 0.46 |
| G027Q | 1.12 |
| G027R | 1.88 |
| G027S | 0.94 |
| G027T | 0.61 |
| G027W | 0.76 |
| K028A | 0.78 |
| K028D | 0.62 |
| K028E | 0.54 |
| K028F | 0.75 |
| K028I | 0.55 |
| K028L | 0.51 |
| K028M | 0.67 |
| K028N | 0.58 |
| K028P | 0.40 |
| K028R | 0.71 |
| K028S | 0.46 |
| K028T | 0.68 |
| K028V | 0.76 |
| K028W | 0.51 |
| F029A | 0.90 |
| F029E | 4.03 |
| F029G | 1.05 |
| F029H | 0.82 |
| F029I | 1.53 |
| F029K | 1.34 |
| F029L | 2.36 |
| F029M | 2.08 |
| F029P | 3.79 |
| F029R | 1.24 |
| F029S | 2.21 |
| F029T | 0.85 |
| F029V | 1.65 |
| F029W | 0.48 |
| D030A | 1.12 |
| D030F | 0.84 |
| D030G | 2.02 |
| D030H | 1.69 |
| D030K | 2.63 |
| D030L | 1.32 |
| D030M | 1.85 |
| D030P | 1.19 |
| D030Q | 0.84 |
| D030R | 1.82 |
| D030S | 1.62 |
| D030T | 0.57 |
| D030V | 0.46 |
| D030W | 0.62 |
| E031A | 2.05 |
| E031C | 2.95 |
| E031G | 1.27 |
| E031H | 2.74 |
| E031I | 3.89 |
| E031K | 3.13 |
| E031L | 2.62 |
| E031P | 1.51 |
| E031R | 2.27 |
| E031S | 1.70 |
| E031T | 3.96 |
| E031V | 4.57 |
| E031W | 1.26 |
| E031Y | 1.13 |
| P032A | 0.92 |
| P032C | 0.40 |
| P032F | 2.71 |
| I326C | 0.39 |
| I331C | 0.27 |
| P032G | 1.60 |
| P032H | 2.08 |
| P032K | 1.04 |
| P032L | 0.82 |
| P032M | 0.67 |
| P032N | 0.70 |
| P032Q | 1.11 |
| P032R | 1.17 |
| P032S | 1.01 |
| P032T | 0.77 |
| P032V | 0.81 |
| P032W | 0.54 |
| P032Y | 1.01 |
| L033G | 0.57 |
| L033M | 0.69 |
| L033P | 0.87 |
| L033Q | 0.45 |
| L033R | 0.61 |
| L033S | 0.48 |
| L033T | 0.45 |
| L033W | 1.58 |
| D034A | 0.38 |
| D034E | 0.58 |
| D034H | 0.41 |
| D034K | 0.54 |
| D034Q | 0.59 |
| D034R | 1.17 |
| D034W | 0.46 |
| M035F | 0.87 |
| M035H | 0.60 |
| M035L | 0.52 |
| M035T | 0.83 |
| M035Y | 0.78 |
| S036A | 0.45 |
| S036D | 0.32 |
| S036G | 0.64 |
| S036H | 0.54 |
| S036K | 0.83 |
| S036L | 0.71 |
| S036R | 1.09 |
| Q347L | 0.39 |
| V351Q | 0.34 |
| S036T | 0.51 |
| L037F | 3.33 |
| L037I | 0.62 |
| L037K | 0.43 |
| L037M | 1.46 |
| L037P | 0.63 |
| L037R | 0.51 |
| L037V | 0.57 |
| F038Y | 1.29 |
| S039A | 1.06 |
| S039L | 0.80 |
| S039N | 2.32 |
| S039Q | 1.10 |
| S039R | 0.56 |
| S039T | 1.57 |
| S039Y | 0.56 |
| F040L | 0.92 |
| F040W | 1.11 |
| I041A | 0.67 |
| I041C | 0.53 |
| I041D | 0.78 |
| I041E | 0.51 |
| I041G | 0.76 |
| I041H | 0.77 |
| I041N | 0.40 |
| I041T | 1.47 |
| I041V | 0.73 |
| I041W | 0.66 |
| G042A | 0.64 |
| S043T | 0.43 |
| P044E | 0.59 |
| R045I | 0.45 |
| R045K | 0.53 |
| I046A | 1.04 |
| I046C | 0.37 |
| I046E | 0.43 |
| I046F | 0.73 |
| I046H | 0.82 |
| I046L | 1.08 |
| I046M | 1.00 |
| I046N | 0.66 |
| I046R | 2.29 |
| I046S | 0.64 |
| I046T | 0.55 |
| I046V | 1.01 |
| I046Y | 0.76 |
| N047A | 0.48 |
| N047D | 0.82 |
| N047F | 1.32 |
| N047G | 0.82 |
| N047H | 1.16 |
| N047K | 0.67 |
| N047M | 0.77 |
| N047Q | 0.69 |
| N047R | 0.84 |
| N047S | 0.85 |
| N047T | 1.49 |
| N047W | 0.63 |
| N047Y | 0.45 |
| A048F | 2.51 |
| A048G | 0.83 |
| A048H | 1.99 |
| A048I | 0.64 |
| A048K | 1.28 |
| A048M | 0.76 |
| A048N | 4.25 |
| A048Q | 1.05 |
| A048R | 0.66 |
| A048S | 1.06 |
| A048V | 0.60 |
| A048Y | 0.81 |
| T049I | 0.42 |
| T049K | 0.85 |
| T049R | 1.41 |
| T049S | 0.92 |
| T049V | 0.45 |
| G050A | 0.93 |
| G050C | 0.41 |
| G050D | 1.37 |
| G050E | 0.78 |
| G050H | 0.74 |
| G050L | 0.43 |
| G050M | 0.47 |
| G050Q | 0.86 |
| G050R | 0.86 |
| G050S | 1.24 |
| G050V | 0.3 |
| G050Y | 0.58 |
| Q051N | 0.60 |
| Q051S | 0.46 |
| G052N | 0.89 |
| G052P | 0.43 |
| G052Q | 3.71 |
| G052R | 0.53 |
| G052S | 1.32 |
| E375I | 0.36 |
| F380V | 0.39 |
| G052T | 0.49 |
| T054A | 0.43 |
| T054F | 0.56 |
| T054N | 0.48 |
| T054Q | 0.91 |
| T054S | 0.70 |
| T054V | 0.66 |
| V058C | 0.55 |
| V058G | 0.54 |
| V058H | 1.09 |
| V058I | 0.57 |
| V058K | 4.08 |
| V058L | 1.54 |
| V058N | 0.49 |
| V058P | 0.90 |
| V058Q | 4.54 |
| V058R | 1.92 |
| V058S | 0.83 |
| V058W | 0.65 |
| V058Y | 1.07 |
| D059Q | 0.40 |
| D059N | 1.27 |
| R060K | 0.69 |
| L061I | 0.42 |
| L061M | 0.73 |
| L061V | 0.59 |
| Y063A | 0.63 |
| Y063H | 1.07 |
| Y063I | 1.03 |
| Y063K | 1.36 |
| Y063L | 1.33 |
| Y063M | 1.32 |
| Y063N | 0.96 |
| Y063R | 1.40 |
| Y063S | 1.00 |
| Y063T | 1.07 |
| Y063V | 0.43 |
| Y063W | 1.53 |
| P065R | 0.57 |
| Y066H | 0.47 |
| Y066R | 0.51 |
| I067F | 1.00 |
| I067L | 0.45 |
| I067R | 0.24 |
| I067V | 1.80 |
| I067Y | 0.55 |
| D068E | 0.72 |
| D068H | 2.06 |
| D068K | 1.08 |
| D068L | 0.43 |
| D068P | 0.50 |
| D068Q | 1.67 |
| D068R | 0.70 |
| D068S | 0.81 |
| D068T | 0.75 |
| S069A | 22.06 |
| S069C | 1.97 |
| S069E | 1.48 |
| S069F | 8.75 |
| S069G | 6.06 |
| S069I | 3.12 |
| S069L | 3.44 |
| S069M | 2.67 |
| S069P | 8.14 |
| S069R | 14.06 |
| S069T | 0.58 |
| S069W | 2.18 |
| S069Y | 2.71 |
| I070A | 27.00 |
| I070C | 2.57 |
| I070F | 5.69 |
| I070G | 6.22 |
| I070H | 9.09 |
| I070K | 14.64 |
| I070L | 3.05 |
| I070N | 6.19 |
| I070P | 3.03 |
| I070R | 13.95 |
| I070S | 3.63 |
| I070T | 5.43 |
| I070V | 6.34 |
| I070Y | 1.26 |
| T071A | 0.86 |
| T071D | 0.50 |
| T071G | 1.41 |
| T071H | 0.93 |
| T071L | 1.09 |
| T071M | 0.89 |
| T071N | 1.21 |
| T071Q | 0.68 |
| T071R | 2.17 |
| T071S | 1.54 |
| G072A | 0.45 |
| G072D | 0.60 |
| S395W | 0.4 |
| G072E | 0.69 |
| G072H | 0.46 |
| G072K | 1.39 |
| G072L | 0.43 |
| G072M | 3.11 |
| G072Q | 2.33 |
| G072R | 0.65 |
| G072S | 0.51 |
| V073A | 1.38 |
| V073C | 0.84 |
| V073D | 0.94 |
| V073G | 1.17 |
| V073H | 1.54 |
| V073K | 1.42 |
| V073L | 1.59 |
| V073M | 0.68 |
| V073Q | 0.96 |
| V073R | 0.72 |
| V073S | 0.86 |
| K297R | 0.34 |
| S401Q | 0.39 |
| V073T | 1.34 |
| V073W | 1.91 |
| T074A | 2.28 |
| T074C | 2.18 |
| T074E | 1.38 |
| T074F | 1.43 |
| T074G | 2.75 |
| T074H | 1.40 |
| T074K | 1.29 |
| T074L | 1.43 |
| T074M | 0.52 |
| T074N | 2.12 |
| T074P | 2.45 |
| T074R | 2.22 |
| T074S | 1.80 |
| T074V | 2.27 |
| T074W | 2.13 |
| V075A | 0.71 |
| V075C | 0.46 |
| V075F | 2.00 |
| V075H | 0.62 |
| V075L | 5.22 |
| V075M | 1.16 |
| V075N | 0.81 |
| V075Q | 1.51 |
| V075R | 3.02 |
| V075S | 0.76 |
| V075T | 4.34 |
| V075Y | 0.63 |
| G077H | 0.32 |
| I079L | 1.44 |
| I079T | 0.79 |
| I079V | 1.01 |
| Q081P | 0.60 |
| K082A | 0.94 |
| K082E | 0.50 |
| K082G | 0.64 |
| K082H | 0.44 |
| K082I | 1.01 |
| K082L | 0.87 |
| K082M | 0.58 |
| K082N | 0.96 |
| K082Q | 0.76 |
| K082R | 0.85 |
| K082S | 0.62 |
| K082T | 0.56 |
| K082Y | 0.32 |
| K082V | 0.57 |
| I083F | 0.57 |
| I083G | 1.05 |
| I083L | 0.93 |
| I083N | 0.82 |
| I083Q | 1.07 |
| I083R | 0.45 |
| I083S | 0.79 |
| I083T | 0.95 |
| I083V | 0.99 |
| S084D | 0.98 |
| S084E | 0.52 |
| S084F | 0.72 |
| S084G | 8.68 |
| S084H | 0.96 |
| S084I | 0.90 |
| S084L | 0.92 |
| S084M | 0.77 |
| S084N | 0.89 |
| S084P | 0.57 |
| S084Q | 0.86 |
| S084R | 1.89 |
| S084T | 0.82 |
| S084W | 0.86 |
| S084Y | 0.30 |
| S221I | 0.35 |
| L085V | 0.42 |
| Q086A | 2.70 |
| Q086D | 0.88 |
| Q086E | 1.18 |
| Q086F | 0.54 |
| Q086G | 1.02 |
| Q086H | 1.70 |
| Q086I | 0.65 |
| Q086K | 0.97 |
| Q086L | 0.92 |
| Q086M | 1.06 |
| Q086N | 1.28 |
| Q086P | 0.42 |
| Q086R | 0.93 |
| Q086S | 0.85 |
| Q086T | 0.58 |
| Q086V | 0.97 |
| Q086W | 1.21 |
| D087A | 1.00 |
| D087C | 1.77 |
| D087E | 0.86 |
| D087G | 1.00 |
| D087H | 0.72 |
| D087I | 0.53 |
| D087L | 0.55 |
| D087M | 0.58 |
| D087P | 0.31 |
| D087Q | 1.05 |
| D087R | 1.28 |
| D087S | 0.99 |
| D087T | 1.70 |
| A412H | 0.39 |
| D413H | 0.31 |
| V414K | 0.3 |
| K415V | 0.39 |
| D087V | 0.66 |
| D087Y | 2.72 |
| L089C | 1.46 |
| L089R | 0.34 |
| L089K | 0.45 |
| L089M | 0.63 |
| D090A | 1.48 |
| D090E | 1.15 |
| D090G | 0.41 |
| D090H | 1.24 |
| D090I | 1.10 |
| D090K | 1.36 |
| D090L | 1.15 |
| D090N | 1.18 |
| D090Q | 1.11 |
| D090R | 1.49 |
| D090S | 1.15 |
| D090T | 1.02 |
| D090W | 0.81 |
| K091A | 0.89 |
| K091Q | 0.43 |
| K091R | 0.67 |
| A092C | 1.97 |
| A092H | 0.22 |
| A092L | 1.29 |
| A092M | 0.86 |
| A092T | 0.70 |
| A092V | 1.09 |
| K093D | 0.71 |
| K093E | 0.83 |
| K093F | 0.50 |
| K093G | 0.97 |
| K093H | 0.61 |
| K093I | 3.25 |
| R248H | 0.4 |
| K093L | 1.53 |
| K093M | 0.70 |
| K093N | 0.71 |
| K093Q | 0.84 |
| K093R | 1.52 |
| K093S | 1.25 |
| K093T | 3.93 |
| K093V | 0.24 |
| K094A | 0.64 |
| K094D | 0.93 |
| K094E | 0.79 |
| K094F | 0.59 |
| K094H | 0.72 |
| K094L | 0.52 |
| K094M | 0.66 |
| K094N | 0.99 |
| K094Q | 1.22 |
| K094R | 3.94 |
| K094S | 0.94 |
| K094T | 1.14 |
| I096D | 0.69 |
| V102K | 1.03 |
| V102L | 0.71 |
| V102M | 0.77 |
| V102N | 1.02 |
| V102Q | 1.03 |
| V102R | 0.94 |
| V102S | 1.41 |
| V102T | 1.26 |
| V102W | 0.76 |
| D103N | 0.39 |
| N104A | 0.69 |
| N104C | 0.41 |
| N104G | 0.48 |
| N104K | 0.88 |
| N104M | 0.61 |
| N104R | 1.25 |
| N104S | 1.03 |
| N104T | 0.71 |
| L105A | 0.54 |
| L105G | 0.51 |
| L105I | 0.94 |
| L105P | 0.84 |
| L105Q | 0.90 |
| L105R | 0.65 |
| L105S | 0.61 |
| L105T | 0.51 |
| L105W | 0.34 |
| L105V | 0.99 |
| G106V | 0.43 |
| M107F | 0.91 |
| M107I | 0.67 |
| M107L | 1.32 |
| A108G | 0.47 |
| I110V | 0.51 |
| E114A | 1.44 |
| E114G | 0.73 |
| E114H | 0.75 |
| E114M | 0.44 |
| E114S | 0.69 |
| P117D | 0.56 |
| T118H | 0.47 |
| T118K | 0.53 |
| T118L | 1.09 |
| T118M | 0.53 |
| T118N | 0.67 |
| T118Q | 3.37 |
| T118V | 0.79 |
| W119F | 0.53 |
| W119P | 0.36 |
| W119Y | 1.08 |
| A120D | 0.76 |
| A120F | 2.62 |
| A120G | 1.03 |
| A120H | 1.11 |
| A120I | 1.33 |
| A120L | 1.25 |
| A120N | 0.81 |
| A120P | 0.42 |
| A120R | 0.82 |
| A120S | 1.21 |
| A120T | 0.62 |
| A120V | 1.53 |
| A120W | 0.59 |
| A120Y | 1.95 |
| N122M | 0.56 |
| K124L | 0.34 |
| K124R | 0.62 |
| P125H | 0.43 |
| P125R | 0.63 |
| P125S | 0.54 |
| D127A | 0.89 |
| D127E | 1.31 |
| D127G | 0.97 |
| D127H | 2.33 |
| D127L | 0.84 |
| D127M | 0.4 |
| D127N | 1.69 |
| D127Q | 1.21 |
| D127R | 0.51 |
| D127S | 0.77 |
| D127T | 1.11 |
| D127V | 0.56 |
| D127W | 0.44 |
| V128A | 0.53 |
| V128C | 0.68 |
| V128G | 0.49 |
| V128I | 1.25 |
| V128K | 1.16 |
| V128L | 0.95 |
| V128Q | 0.55 |
| V128R | 0.74 |
| V128S | 0.53 |
| V128W | 0.50 |
| K130I | 0.50 |
| K130R | 1.42 |
| N131C | 0.60 |
| N131E | 0.44 |
| N131F | 0.63 |
| N131G | 2.47 |
| N131H | 0.80 |
| N131I | 1.40 |
| N131L | 0.82 |
| N131M | 0.99 |
| N131Q | 1.24 |
| N131R | 2.81 |
| N131S | 0.76 |
| N131T | 1.02 |
| N131V | 2.08 |
| N131Y | 0.85 |
| R132A | 0.68 |
| R132C | 0.58 |
| R132E | 0.70 |
| R132F | 0.60 |
| R132H | 0.66 |
| K279A | 0.27 |
| E285A | 0.34 |
| R132I | 0.56 |
| R132K | 1.05 |
| R132L | 0.76 |
| R132N | 1.28 |
| R132Q | 0.69 |
| R132S | 0.79 |
| R132T | 0.61 |
| R132V | 0.73 |
| R132Y | 0.78 |
| S133I | 0.54 |
| I134L | 1.04 |
| I134T | 0.60 |
| I134V | 1.08 |
| E135A | 0.99 |
| E135C | 0.77 |
| E135D | 2.68 |
| E135F | 0.73 |
| E442L | 0.4 |
| E135G | 2.79 |
| E135H | 0.79 |
| E135K | 1.15 |
| E135L | 0.82 |
| E135N | 0.56 |
| E135Q | 1.59 |
| E135R | 2.08 |
| E135S | 1.13 |
| E135W | 0.63 |
| E135Y | 0.50 |
| L136A | 0.73 |
| L136C | 0.56 |
| L136D | 0.47 |
| L136F | 0.96 |
| L136H | 1.00 |
| L136I | 0.65 |
| L136M | 1.05 |
| L136N | 0.48 |
| L136Q | 0.61 |
| L136R | 0.74 |
| L136S | 0.80 |
| L136T | 0.72 |
| L136W | 1.11 |
| V137A | 0.48 |
| V137I | 1.01 |
| V137T | 0.51 |
| Q138A | 0.69 |
| Q138C | 0.65 |
| Q138H | 0.71 |
| Q138I | 0.54 |
| Q138L | 0.59 |
| Q138M | 0.68 |
| Q138N | 0.61 |
| Q138R | 0.53 |
| Q138S | 0.48 |
| Q138W | 0.41 |
| Q138Y | 0.60 |
| Q139A | 0.92 |
| Q139C | 0.44 |
| Q139D | 0.48 |
| Q139E | 0.94 |
| Q139F | 0.53 |
| Q139G | 0.65 |
| Q139H | 0.56 |
| Q139K | 0.73 |
| Q139L | 0.70 |
| Q139M | 0.95 |
| Q139R | 0.79 |
| Q139S | 0.81 |
| Q139T | 1.31 |
| Q139V | 0.77 |
| Q140A | 0.96 |
| Q140C | 0.50 |
| Q140D | 0.59 |
| Q140F | 0.66 |
| Q140G | 0.73 |
| Q140H | 0.84 |
| Q140I | 0.75 |
| Q140K | 0.93 |
| Q140L | 0.51 |
| Q140M | 0.80 |
| Q140R | 0.85 |
| Q140V | 0.61 |
| Q140W | 0.59 |
| Q140Y | 0.41 |
| N141A | 1.12 |
| N141D | 1.09 |
| N141E | 0.67 |
| N141F | 0.81 |
| N141G | 1.15 |
| N141H | 2.03 |
| N002I | 0.37 |
| G297A | 0.57 |
| N141L | 0.61 |
| N141M | 0.48 |
| N141Q | 1.16 |
| N141R | 1.40 |
| N141S | 0.72 |
| N141T | 0.45 |
| N141V | 0.50 |
| N141W | 0.83 |
| N141Y | 1.55 |
| V142C | 0.61 |
| V142D | 0.71 |
| V142E | 0.87 |
| V142G | 0.98 |
| V142H | 1.11 |
| V142I | 0.81 |
| V142K | 1.40 |
| V142L | 0.75 |
| V142M | 0.76 |
| V142N | 0.98 |
| V142P | 0.88 |
| V142Q | 1.04 |
| V142R | 1.53 |
| V142S | 0.93 |
| V142T | 1.19 |
| Q143E | 0.77 |
| Q143G | 0.62 |
| Q143I | 0.44 |
| Q143K | 1.30 |
| I009Q | 0.4 |
| Q143L | 0.56 |
| Q143N | 0.73 |
| Q143V | 0.57 |
| L144T | 1.02 |
| L144W | 0.79 |
| S145A | 0.58 |
| S145C | 0.44 |
| S145D | 0.48 |
| S145E | 0.56 |
| S145G | 0.94 |
| S145H | 0.56 |
| S145L | 0.44 |
| S145M | 0.56 |
| S145N | 0.58 |
| S145P | 1.04 |
| S145R | 0.97 |
| L146A | 0.52 |
| L146C | 0.42 |
| G305N | 0.36 |
| M310Y | 0.38 |
| L146E | 0.50 |
| L146G | 0.62 |
| L146H | 0.78 |
| L146I | 0.82 |
| L146K | 0.84 |
| L146N | 0.57 |
| L146P | 0.93 |
| L146Q | 0.84 |
| L146R | 1.47 |
| L146S | 0.71 |
| L146T | 0.74 |
| L146V | 0.84 |
| L146Y | 0.80 |
| S312K | 0.38 |
| T147A | 1.20 |
| T147C | 0.47 |
| T147D | 0.71 |
| T147F | 1.24 |
| T147G | 1.05 |
| T147I | 0.85 |
| T147L | 1.30 |
| T147M | 0.79 |
| T147P | 1.09 |
| T147Q | 1.29 |
| T147R | 2.11 |
| T147S | 1.27 |
| T147V | 2.04 |
| T147W | 0.97 |
| T147Y | 1.04 |
| E148C | 0.66 |
| E148F | 0.42 |
| E148G | 1.05 |
| E148H | 1.24 |
| E148I | 0.73 |
| E148K | 1.63 |
| E148L | 0.85 |
| E148Q | 1.44 |
| E148R | 0.97 |
| E148S | 1.15 |
| E148T | 0.82 |
| E148V | 0.99 |
| E148W | 0.43 |
| E148Y | 0.95 |
| A149C | 1.15 |
| A149G | 0.52 |
| A149K | 0.51 |
| A149L | 0.88 |
| A149M | 0.88 |
| A149Q | 1.15 |
| A149R | 1.02 |
| A149S | 1.08 |
| A149T | 1.24 |
| A149V | 1.34 |
| T150A | 1.21 |
| T150C | 0.70 |
| T150D | 1.24 |
| T150E | 1.05 |
| T150F | 0.71 |
| T150G | 2.19 |
| T150I | 0.52 |
| T150L | 0.70 |
| T150N | 0.91 |
| T150P | 0.88 |
| T150R | 0.90 |
| T150S | 0.92 |
| T150W | 1.25 |
| T150Y | 1.36 |
| E151A | 1.27 |
| E151C | 1.00 |
| E151G | 1.06 |
| E151H | 1.34 |
| E151K | 2.05 |
| E151L | 1.03 |
| E151M | 1.26 |
| E151N | 0.95 |
| E151Q | 2.01 |
| D320L | 0.37 |
| E151R | 1.61 |
| E151S | 1.28 |
| E151T | 1.21 |
| E151V | 1.38 |
| E151W | 1.31 |
| E151Y | 1.31 |
| K152A | 0.51 |
| K152C | 0.52 |
| K152F | 0.61 |
| K152I | 0.65 |
| K152M | 0.75 |
| K152R | 1.85 |
| K152T | 1.20 |
| K152V | 0.82 |
| K152Y | 0.67 |
| A153I | 0.93 |
| A153L | 0.51 |
| K154R | 0.86 |
| K154T | 0.83 |
| K154V | 0.46 |
| Q155A | 0.91 |
| Q155C | 0.60 |
| Q155D | 1.49 |
| Q155F | 0.70 |
| Q155G | 1.61 |
| Q155H | 1.03 |
| Q155K | 1.57 |
| Q155L | 0.86 |
| Q155M | 0.97 |
| Q155R | 1.27 |
| Q155S | 0.77 |
| Q155T | 0.76 |
| Q155V | 0.73 |
| Q155W | 0.91 |
| E156A | 0.79 |
| E156D | 1.95 |
| E156G | 0.49 |
| E156I | 0.51 |
| E156L | 0.43 |
| E156M | 0.87 |
| E156Q | 0.84 |
| E156R | 0.43 |
| E156S | 0.62 |
| E156T | 0.69 |
| E156V | 0.45 |
| E156W | 0.49 |
| F157W | 0.61 |
| E158A | 0.56 |
| E158F | 0.51 |
| E158H | 0.54 |
| I326S | 0.95 |
| I331E | 0.34 |
| E158L | 0.44 |
| E158Q | 1.25 |
| E158S | 0.95 |
| K159A | 0.64 |
| K159D | 0.52 |
| K159E | 0.49 |
| K159H | 0.74 |
| K159L | 0.62 |
| K159M | 0.66 |
| K159N | 0.73 |
| K159Q | 0.92 |
| K159R | 0.88 |
| K159S | 0.67 |
| K159V | 0.41 |
| A160C | 0.61 |
| A160F | 0.79 |
| A160G | 0.75 |
| A160H | 0.47 |
| A160I | 0.43 |
| A160K | 0.91 |
| A160L | 0.67 |
| M035Q | 0.37 |
| A160M | 0.77 |
| A160N | 0.56 |
| A160Q | 0.65 |
| A160R | 0.89 |
| A160S | 1.35 |
| A160V | 0.73 |
| A160Y | 1.07 |
| G161A | 0.99 |
| G161C | 0.44 |
| G161D | 0.86 |
| G161E | 0.49 |
| G161R | 0.48 |
| S036N | 0.38 |
| G161S | 0.77 |
| G161V | 0.42 |
| K162A | 0.50 |
| K162D | 0.77 |
| K162E | 0.51 |
| V351C | 0.35 |
| W357K | 0.36 |
| K162G | 0.56 |
| K162H | 0.62 |
| K162L | 0.54 |
| K162M | 1.04 |
| K162P | 0.64 |
| K162Q | 0.58 |
| K162R | 0.52 |
| K162S | 0.47 |
| K162V | 0.52 |
| K162W | 1.01 |
| K162Y | 0.72 |
| D163A | 1.52 |
| D163E | 1.63 |
| D163G | 1.15 |
| D163K | 1.90 |
| D163L | 1.18 |
| D163Q | 1.40 |
| D163R | 1.80 |
| D163S | 1.34 |
| D163T | 1.13 |
| D163V | 0.76 |
| F164L | 1.13 |
| F164M | 1.66 |
| F164V | 1.23 |
| S043N | 0.34 |
| F164W | 0.72 |
| L165A | 0.48 |
| L165D | 5.79 |
| L165F | 1.23 |
| L165N | 2.19 |
| L165R | 0.59 |
| L165S | 1.31 |
| L165V | 1.22 |
| L165W | 1.14 |
| A371G | 0.38 |
| L165Y | 0.66 |
| V166A | 2.85 |
| V166C | 1.16 |
| V166E | 1.28 |
| V166F | 1.67 |
| V166G | 1.11 |
| V166H | 1.74 |
| V166L | 4.38 |
| V166Q | 3.61 |
| V166R | 5.56 |
| V166T | 4.26 |
| V166W | 1.26 |
| V166Y | 2.08 |
| E167A | 0.84 |
| E167D | 0.69 |
| E167G | 0.60 |
| E167H | 0.89 |
| E167K | 0.91 |
| E167M | 0.87 |
| E167N | 0.83 |
| E167P | 0.58 |
| E167R | 1.02 |
| E167S | 1.17 |
| E167T | 0.59 |
| E167Y | 0.55 |
| T168H | 0.46 |
| I169L | 2.08 |
| I169R | 0.54 |
| I169V | 0.74 |
| K170N | 0.72 |
| K170R | 2.58 |
| K170V | 0.58 |
| L171I | 0.73 |
| L171V | 0.64 |
| G172A | 1.20 |
| G172C | 1.03 |
| K173N | 0.44 |
| K173R | 0.82 |
| L174A | 1.20 |
| L174G | 0.40 |
| L174K | 2.39 |
| L174M | 0.79 |
| L174N | 1.36 |
| L174Q | 0.99 |
| L174R | 1.50 |
| L174S | 0.85 |
| L174T | 1.12 |
| L174V | 0.62 |
| L174W | 0.78 |
| L174Y | 1.06 |
| L175E | 0.43 |
| Q051A | 0.34 |
| L175H | 0.57 |
| L175T | 1.43 |
| L175V | 0.94 |
| L175Y | 0.66 |
| R176K | 0.67 |
| N178G | 0.85 |
| N178K | 0.85 |
| N178M | 0.88 |
| K376L | 0.37 |
| F380T | 0.39 |
| N178R | 1.10 |
| H179A | 1.06 |
| H179C | 0.94 |
| H179E | 0.62 |
| H179G | 0.86 |
| H179I | 0.90 |
| H179K | 1.39 |
| H179L | 0.73 |
| H179M | 0.63 |
| H179N | 0.96 |
| H179P | 0.44 |
| H179R | 0.96 |
| H179S | 0.51 |
| H179T | 0.43 |
| H179V | 0.42 |
| L180F | 0.59 |
| L180G | 0.62 |
| L180K | 0.44 |
| L180M | 0.64 |
| W181M | 0.88 |
| L061F | 0.3 |
| W181Q | 0.88 |
| G182L | 0.90 |
| Y183L | 0.70 |
| F186Y | 0.59 |
| H192S | 0.49 |
| H192T | 0.50 |
| H193G | 0.68 |
| H193Q | 0.82 |
| H193S | 0.42 |
| H193Y | 0.58 |
| K195A | 0.51 |
| K195G | 0.45 |
| K195H | 0.45 |
| K195I | 0.50 |
| K195L | 0.45 |
| K195N | 0.74 |
| K195Q | 0.71 |
| K195R | 0.85 |
| K195S | 0.42 |
| K195T | 0.58 |
| K195W | 0.49 |
| K196E | 0.43 |
| D068G | 0.37 |
| K196G | 0.41 |
| K196L | 0.65 |
| K196R | 0.58 |
| K196S | 0.68 |
| K196T | 1.18 |
| K196W | 0.55 |
| P197A | 0.81 |
| P197D | 0.58 |
| P197E | 0.52 |
| P197F | 0.48 |
| P197G | 0.75 |
| P197H | 0.62 |
| P197K | 0.99 |
| P197L | 0.56 |
| P197M | 1.03 |
| P197Q | 0.69 |
| P197R | 0.58 |
| P197S | 0.70 |
| P197T | 0.41 |
| G198A | 0.80 |
| G198D | 1.99 |
| G198E | 0.49 |
| G198H | 0.84 |
| G198L | 0.48 |
| G198N | 0.80 |
| G198Q | 0.55 |
| G198R | 0.58 |
| G198S | 0.76 |
| G198T | 0.41 |
| G198Y | 0.81 |
| N200D | 0.46 |
| S202M | 0.40 |
| F204P | 0.63 |
| N205A | 1.30 |
| N205D | 0.85 |
| N205E | 1.94 |
| N205F | 0.52 |
| N205G | 0.79 |
| N205K | 0.76 |
| N205M | 0.58 |
| N205P | 0.75 |
| N205R | 0.54 |
| N205S | 0.80 |
| N205T | 0.85 |
| N205V | 0.49 |
| N205W | 0.41 |
| V206H | 0.50 |
| V206I | 0.94 |
| V206K | 1.75 |
| V206L | 1.57 |
| S395T | 0.39 |
| V206M | 0.43 |
| V206R | 1.30 |
| V206S | 0.72 |
| G072Y | 0.35 |
| V206T | 0.59 |
| I208A | 0.62 |
| I208C | 0.48 |
| I208K | 0.91 |
| I208L | 0.84 |
| I208M | 0.88 |
| I208Q | 0.77 |
| I208R | 1.14 |
| I208S | 0.62 |
| I208T | 1.01 |
| I208V | 1.07 |
| K209A | 0.53 |
| K209E | 0.46 |
| K209G | 0.44 |
| K209N | 0.50 |
| F398L | 0.35 |
| S404T | 0.37 |
| K209R | 0.68 |
| K209S | 0.50 |
| K209T | 0.50 |
| D212N | 1.52 |
| D212S | 0.93 |
| D212T | 0.76 |
| D213A | 0.85 |
| D213E | 0.79 |
| D213G | 0.81 |
| D213H | 0.75 |
| D213K | 0.82 |
| D213L | 0.56 |
| D213M | 1.56 |
| D213N | 1.53 |
| D213Q | 1.04 |
| D213R | 0.92 |
| D213V | 0.47 |
| D213W | 0.49 |
| D213Y | 0.49 |
| L214Q | 0.57 |
| S215A | 0.74 |
| S215D | 0.62 |
| S215E | 0.74 |
| S215G | 0.88 |
| S215H | 0.91 |
| S215K | 0.99 |
| S215L | 0.60 |
| S215M | 1.77 |
| S215Q | 0.79 |
| G077K | 0.32 |
| S215R | 0.71 |
| S215T | 0.80 |
| S215V | 0.69 |
| S215W | 0.52 |
| W216Y | 0.48 |
| L217M | 0.51 |
| W218F | 0.57 |
| N219A | 1.29 |
| N219C | 0.43 |
| N219D | 0.75 |
| N219E | 0.95 |
| N219H | 0.97 |
| N219I | 0.60 |
| N219K | 1.45 |
| N219L | 0.72 |
| N219M | 1.02 |
| I083H | 0.4 |
| N219R | 1.10 |
| N219S | 2.48 |
| N219T | 0.82 |
| N219W | 0.48 |
| E220A | 0.75 |
| E220H | 1.40 |
| E220I | 1.34 |
| E220L | 1.45 |
| E220S | 0.62 |
| E220T | 0.91 |
| E220V | 1.35 |
| S221A | 0.72 |
| S221C | 0.59 |
| S221M | 0.46 |
| S221Q | 1.37 |
| S221T | 0.94 |
| S221V | 1.04 |
| T222D | 0.43 |
| T222F | 0.43 |
| T222G | 0.49 |
| T222K | 0.75 |
| T222L | 0.64 |
| T222N | 0.80 |
| T222R | 0.75 |
| E220D | 0.39 |
| T222I | 0.4 |
| T222S | 0.63 |
| T222V | 0.79 |
| L224I | 0.61 |
| L230I | 0.87 |
| N231T | 1.10 |
| T232F | 0.73 |
| T232S | 0.76 |
| Q233A | 0.71 |
| Q233F | 0.53 |
| Q233G | 0.46 |
| Q233K | 1.69 |
| Q233L | 0.69 |
| Q233R | 1.50 |
| Q233Y | 0.50 |
| Q234M | 1.65 |
| S235A | 0.47 |
| S235E | 1.00 |
| S235G | 0.95 |
| S235H | 0.44 |
| S235K | 0.53 |
| S235T | 0.66 |
| P236A | 1.07 |
| P236G | 1.09 |
| P236H | 0.46 |
| P236K | 0.71 |
| P236R | 3.09 |
| Q234L | 0.40 |
| P236S | 0.91 |
| V237A | 0.90 |
| V237E | 1.93 |
| V237F | 0.41 |
| A412Q | 0.35 |
| A413Q | 0.38 |
| V414L | 0.36 |
| D418G | 0.45 |
| V237H | 0.75 |
| V237L | 1.12 |
| V237N | 0.67 |
| L089W | 0.26 |
| V237Q | 1.46 |
| V237R | 0.71 |
| V237S | 1.03 |
| V237T | 1.01 |
| V237W | 0.52 |
| A238D | 0.75 |
| A238E | 0.59 |
| A238H | 0.60 |
| A238K | 0.60 |
| A238Q | 1.02 |
| A238R | 0.49 |
| A238S | 2.62 |
| A238T | 0.44 |
| T240K | 1.13 |
| T240A | 0.48 |
| T240M | 0.48 |
| T240P | 0.56 |
| T240Q | 0.75 |
| T240R | 0.91 |
| A239N | 0.32 |
| T240S | 0.74 |
| T240V | 0.77 |
| Y242F | 1.08 |
| N245H | 0.50 |
| V247I | 2.01 |
| V247L | 0.83 |
| V247M | 0.52 |
| R248A | 0.43 |
| R248W | 0.52 |
| R248Y | 0.67 |
| I251Y | 0.37 |
| I251L | 0.58 |
| I251M | 0.43 |
| V253I | 0.76 |
| K255A | 0.40 |
| K255N | 0.52 |
| K255Q | 0.91 |
| K255R | 0.71 |
| K093P | 0.38 |
| K255S | 0.43 |
| I256A | 0.42 |
| I256H | 0.51 |
| I256L | 0.64 |
| I256V | 0.51 |
| P257A | 0.82 |
| P257G | 0.51 |
| P257I | 1.07 |
| P257K | 0.92 |
| P257L | 0.69 |
| P257M | 0.90 |
| P257N | 0.69 |
| P257Q | 0.61 |
| S261R | 1.19 |
| S261T | 0.66 |
| S261V | 0.48 |
| S261W | 0.44 |
| L263A | 0.76 |
| L263K | 2.73 |
| L263M | 0.89 |
| L263R | 1.63 |
| L263T | 0.49 |
| N104I | 0.35 |
| L263V | 0.75 |
| P264A | 0.43 |
| P264H | 0.60 |
| V265I | 0.58 |
| F266Y | 0.58 |
| A267M | 0.45 |
| A267T | 1.34 |
| T269A | 1.63 |
| T269C | 0.75 |
| T269D | 0.76 |
| T269S | 1.01 |
| R270M | 0.46 |
| R270N | 0.52 |
| R270S | 0.69 |
| I271F | 0.72 |
| I271G | 1.29 |
| L105C | 0.33 |
| I271L | 10.62 |
| V272E | 0.39 |
| I271M | 3.24 |
| I271S | 0.42 |
| I271V | 1.05 |
| V272D | 1.36 |
| V272R | 0.74 |
| V272S | 0.96 |
| V272T | 1.61 |
| F273H | 1.41 |
| F273T | 0.48 |
| F273Y | 0.90 |
| T274A | 0.51 |
| T274F | 1.28 |
| T274S | 0.62 |
| Q276C | 0.88 |
| Q276D | 1.69 |
| Q276E | 1.05 |
| Q276H | 1.20 |
| Q276I | 0.51 |
| Q276L | 0.48 |
| W119Q | 0.72 |
| Q276M | 1.14 |
| Q276R | 1.30 |
| Q276S | 1.63 |
| Q276Y | 1.94 |
| V277A | 0.65 |
| V277C | 0.41 |
| V277D | 0.79 |
| V277E | 1.02 |
| V277G | 1.18 |
| V277H | 1.09 |
| V277K | 1.51 |
| V277M | 0.94 |
| V277N | 1.15 |
| V277Q | 0.82 |
| V277R | 1.63 |
| V277S | 0.83 |
| K124H | 0.35 |
| V277T | 1.94 |
| V277Y | 0.66 |
| L278A | 1.13 |
| L278E | 1.03 |
| L278F | 1.26 |
| L278G | 1.33 |
| L278H | 4.50 |
| L278I | 0.93 |
| L278K | 1.75 |
| D275V | 0.4 |
| L278N | 1.74 |
| L278R | 5.87 |
| L278S | 1.67 |
| L278T | 1.66 |
| L278V | 0.44 |
| L278Y | 1.51 |
| K279H | 0.44 |
| K279Q | 0.84 |
| K279R | 1.10 |
| K279T | 0.86 |
| F280G | 0.47 |
| F280Q | 0.43 |
| S282D | 0.41 |
| S282G | 0.54 |
| S282M | 2.64 |
| S282Q | 0.41 |
| Q283E | 0.63 |
| Q283P | 1.18 |
| Q283R | 0.59 |
| Q283S | 1.73 |
| Q283T | 0.65 |
| D284A | 0.58 |
| D284E | 1.21 |
| D284G | 0.60 |
| D284H | 0.51 |
| D284L | 0.50 |
| D284M | 0.56 |
| D284N | 0.40 |
| D284Q | 0.95 |
| D284S | 0.99 |
| E285F | 0.47 |
| E285G | 0.52 |
| E285H | 1.30 |
| E285M | 0.43 |
| E285N | 0.40 |
| E285Q | 0.59 |
| E285Y | 0.99 |
| L286S | 0.46 |
| D284T | 0.39 |
| L286R | 0.53 |
| V287I | 0.51 |
| V287T | 0.50 |
| Y288L | 0.79 |
| Y288W | 0.49 |
| T289K | 0.75 |
| T289S | 0.48 |
| F290I | 0.41 |
| F290M | 1.03 |
| G291Q | 0.80 |
| G291R | 0.45 |
| G291S | 0.41 |
| G291V | 1.63 |
| E292A | 0.66 |
| E292C | 0.71 |
| E292F | 0.90 |
| E292G | 0.41 |
| E292H | 1.26 |
| E442W | 0.38 |
| E292K | 1.27 |
| E292N | 0.99 |
| E292P | 1.05 |
| E292R | 0.42 |
| E292V | 1.28 |
| E292W | 0.83 |
| T293A | 1.90 |
| T293C | 1.67 |
| T293D | 1.46 |
| V137C | 0.37 |
| V137S | 0.36 |
| V137L | 0.21 |
| Q143C | 0.28 |
| L144R | 0.26 |
| K152W | 0.37 |
| A153S | 0.34 |
| K154I | 0.38 |
| E156C | 0.35 |
| E158G | 0.37 |
| K159G | 0.38 |
| A160W | 0.39 |
| G161V | 0.42 |
| D163W | 0.38 |
| D163F | 0.39 |
| L165C | 0.27 |
| V166N | 0.47 |
| E167F | 0.31 |
| K170A | 0.40 |
| K170Q | 0.40 |
| K173Q | 0.32 |
| L174H | 0.38 |
| R176L | 0.40 |
| P177V | 0.36 |
| L180I | 0.38 |
| W181K | 0.29 |
| Y183E | 0.32 |
| Y184W | 0.39 |
| H193R | 0.33 |
| H193F | 0.38 |
| K195V | 0.36 |
| K196N | 0.39 |
| K196Y | 0.39 |
| P197W | 0.39 |
| G198W | 0.29 |
| N200T | 0.37 |
| F204W | 0.39 |
| N205L | 0.39 |
| N205Y | 0.4 |
| V206Q | 0.33 |
| K209F | 0.4 |
| K209L | 0.38 |
| N211L | 0.41 |
| N211W | 0.51 |
| W218M | 0.38 |
| W218V | 0.28 |
| T293F | 1.94 |
| T293G | 1.00 |
| T293K | 1.35 |
| T293L | 1.00 |
| T293M | 2.29 |
| T293P | 1.64 |
| T293Q | 1.83 |
| T293S | 0.89 |
| T293V | 2.15 |
| T293Y | 1.49 |
| V294M | 0.41 |
| A298G | 0.43 |
| A298I | 0.41 |
| G300R | 0.42 |
| I301A | 0.88 |
| I301V | 0.88 |
| V287N | 0.35 |
| V302W | 0.46 |
| V302I | 0.45 |
| I303V | 0.47 |
| W304G | 1.13 |
| W304I | 1.17 |
| G305D | 1.00 |
| G305E | 1.62 |
| T306D | 0.76 |
| T306E | 0.52 |
| T306S | 1.02 |
| L307K | 0.43 |
| L307N | 0.76 |
| L307Q | 0.61 |
| L307S | 0.86 |
| L307T | 1.08 |
| L307V | 0.48 |
| L307W | 0.64 |
| L307Y | 0.60 |
| S308D | 0.92 |
| S308G | 1.73 |
| S308H | 1.15 |
| S308K | 1.33 |
| S308N | 2.33 |
| S308P | 0.65 |
| S308R | 1.34 |
| S308T | 0.72 |
| I309D | 0.72 |
| I309E | 1.99 |
| I309G | 1.44 |
| I303D | 0.34 |
| I309H | 1.30 |
| I309K | 0.98 |
| I309L | 1.72 |
| I309M | 1.47 |
| I309N | 3.11 |
| I309Q | 1.64 |
| I309R | 2.27 |
| I309S | 1.16 |
| I309T | 2.09 |
| I309V | 0.60 |
| I309W | 0.88 |
| M310A | 1.50 |
| M310G | 2.73 |
| M310Q | 0.59 |
| M310R | 0.50 |
| M310S | 1.61 |
| M310V | 0.70 |
| R311G | 0.53 |
| L307G | 0.32 |
| R311G | 0.54 |
| R311H | 0.48 |
| R311K | 0.72 |
| R311Q | 0.43 |
| R311S | 0.84 |
| R311T | 0.52 |
| S312G | 0.49 |
| S312N | 1.26 |
| S312T | 0.75 |
| M313A | 1.34 |
| M313E | 0.63 |
| M313G | 0.56 |
| M313H | 1.23 |
| M313K | 2.85 |
| S312L | 0.38 |
| M313L | 1.05 |
| M313P | 1.11 |
| M313R | 2.30 |
| M313S | 0.88 |
| M313T | 0.67 |
| M313V | 0.99 |
| M313Y | 1.12 |
| K314A | 0.82 |
| K314D | 0.53 |
| K314H | 1.10 |
| K314I | 0.54 |
| K314N | 0.57 |
| K314Q | 0.62 |
| K314R | 0.95 |
| K314S | 0.61 |
| K314T | 0.61 |
| K314Y | 0.45 |
| S315A | 0.85 |
| S315E | 0.41 |
| S315G | 0.72 |
| S315H | 2.04 |
| S315K | 0.62 |
| S315L | 0.42 |
| S315M | 0.63 |
| S315R | 1.04 |
| S315T | 0.97 |
| S315Y | 0.50 |
| C316D | 0.41 |
| L317A | 1.27 |
| L317D | 0.61 |
| L317H | 1.05 |
| L317I | 1.76 |
| L317K | 5.11 |
| L317M | 1.20 |
| L317N | 0.73 |
| L317Q | 1.67 |
| L317R | 2.41 |
| L317S | 1.03 |
| L317T | 0.93 |
| L317W | 0.84 |
| L318D | 0.46 |
| L318F | 0.51 |
| L318G | 0.49 |
| L318H | 0.45 |
| L318I | 0.70 |
| L318K | 1.36 |
| L318M | 1.68 |
| L318N | 0.52 |
| L318Q | 0.71 |
| L318R | 1.34 |
| L318S | 0.71 |
| L318T | 0.63 |
| D320E | 0.78 |
| D320G | 0.83 |
| D320H | 1.75 |
| D320I | 1.00 |
| D320K | 6.42 |
| D320M | 0.79 |
| D320N | 0.52 |
| D320R | 3.19 |
| D320S | 1.19 |
| D320W | 0.40 |
| D320V | 0.35 |
| D320Y | 0.86 |
| N321A | 1.01 |
| N321D | 1.25 |
| N321H | 0.92 |
| N321K | 1.29 |
| N321R | 1.23 |
| N321S | 1.26 |
| N321T | 0.64 |
| N321Y | 0.40 |
| M323F | 0.64 |
| M323I | 0.55 |
| M323L | 0.55 |
| E324A | 0.59 |
| E324D | 1.15 |
| E324H | 0.79 |
| E324M | 0.50 |
| E324N | 1.01 |
| E324R | 2.28 |
| E324S | 0.62 |
| T325A | 1.87 |
| T325D | 1.78 |
| T325E | 4.03 |
| T325G | 4.21 |
| T325H | 3.45 |
| T325K | 4.37 |
| T325M | 2.11 |
| T325N | 4.64 |
| T325Q | 5.08 |
| T325S | 3.19 |
| T325V | 1.24 |
| T325W | 0.62 |
| I326K | 0.95 |
| I326L | 1.50 |
| I326V | 6.29 |
| I326Y | 0.77 |
| L327M | 0.52 |
| N328A | 0.67 |
| N328C | 1.25 |
| N328G | 0.56 |
| N328H | 0.88 |
| N328I | 1.85 |
| N328K | 2.12 |
| N328L | 2.01 |
| N328Q | 1.13 |
| N328R | 0.68 |
| N328S | 2.22 |
| N328T | 0.59 |
| N328V | 1.16 |
| N328Y | 1.66 |
| I331V | 0.94 |
| N328W | 0.33 |
| V334T | 0.39 |
| V334P | 0.46 |
| T335S | 0.47 |
| A338Q | 0.63 |
| K339M | 0.61 |
| S342A | 0.68 |
| Q343T | 0.49 |
| Q343V | 0.51 |
| Q347A | 0.78 |
| Q347E | 0.78 |
| Q347G | 2.68 |
| Q347M | 0.61 |
| Q347R | 0.55 |
| Q347S | 2.38 |
| E348D | 0.67 |
| E348G | 0.55 |
| E348S | 0.44 |
| Q349A | 0.47 |
| Q349E | 0.83 |
| Q349K | 0.93 |
| Q349M | 0.70 |
| Q349N | 0.44 |
| M035V | 0.37 |
| Q349R | 0.73 |
| Q349T | 0.49 |
| V351A | 1.14 |
| V351S | 0.92 |
| I353T | 0.42 |
| I353V | 1.61 |
| N356A | 0.41 |
| N356D | 0.79 |
| N356H | 0.82 |
| N356S | 0.46 |
| W357A | 0.80 |
| W357C | 0.67 |
| L037W | 0.36 |
| W357S | 0.41 |
| W357T | 0.62 |
| N358C | 0.66 |
| N358G | 0.41 |
| N358T | 0.58 |
| V351I | 0.36 |
| N358L | 0.38 |
| S359D | 0.45 |
| S359E | 1.05 |
| S359H | 0.44 |
| S359K | 0.66 |
| S359M | 0.63 |
| S359T | 2.11 |
| S359V | 0.65 |
| S360T | 0.50 |
| P367A | 0.55 |
| P367C | 0.83 |
| P367G | 0.47 |
| P367K | 0.57 |
| P367R | 0.46 |
| P367S | 0.52 |
| D368A | 1.34 |
| D368E | 1.28 |
| D368G | 0.49 |
| D368H | 0.96 |
| D368K | 1.31 |
| D368L | 0.64 |
| D368M | 0.78 |
| D368R | 1.31 |
| D368S | 0.93 |
| D368T | 0.80 |
| D361H | 0.37 |
| D368V | 0.41 |
| N369H | 1.33 |
| N369R | 0.55 |
| N369S | 0.54 |
| A371E | 1.05 |
| A371F | 0.52 |
| A371H | 1.20 |
| A371I | 0.50 |
| A371K | 1.76 |
| L374W | 0.34 |
| A371L | 0.57 |
| A371M | 0.57 |
| A371R | 1.51 |
| A371S | 1.45 |
| A371V | 0.94 |
| Q373A | 0.65 |
| Q373E | 0.81 |
| Q373F | 0.62 |
| Q373K | 0.73 |
| Q373L | 0.84 |
| Q373M | 1.43 |
| Q373R | 0.68 |
| Q373S | 0.87 |
| Q373V | 1.05 |
| L374A | 0.60 |
| L374H | 1.42 |
| L374I | 0.80 |
| L374M | 1.11 |
| L374N | 0.43 |
| L374P | 0.43 |
| L374R | 0.83 |
| L374S | 0.58 |
| L374T | 0.47 |
| L374V | 0.56 |
| L374Y | 0.66 |
| E375A | 0.42 |
| E375G | 0.90 |
| E375K | 1.49 |
| E375L | 0.46 |
| E375M | 0.54 |
| E375N | 0.81 |
| E375R | 0.43 |
| E375S | 0.77 |
| E375T | 1.17 |
| K376A | 0.95 |
| K376D | 0.78 |
| K376E | 0.88 |
| K376M | 0.46 |
| K376Q | 0.69 |
| K376R | 0.67 |
| K376S | 0.80 |
| K376T | 0.53 |
| K376V | 0.58 |
| K376Y | 0.42 |
| G377D | 1.35 |
| G377E | 0.59 |
| G377H | 1.49 |
| G377K | 1.50 |
| G377P | 2.30 |
| G377R | 1.28 |
| G377S | 1.80 |
| Q051R | 0.36 |
| G377T | 3.83 |
| G378K | 1.22 |
| G378N | 0.64 |
| G378R | 1.03 |
| K379G | 0.52 |
| K379H | 0.57 |
| K379R | 0.74 |
| K379S | 0.46 |
| K379T | 0.4 |
| M035Q | 0.37 |
| F380I | 0.56 |
| F380L | 0.67 |
| F380P | 0.47 |
| F380W | 2.15 |
| F380Y | 1.50 |
| T381H | 0.48 |
| T381K | 1.06 |
| T381N | 0.51 |
| T381Q | 0.84 |
| T381R | 0.87 |
| T381S | 0.87 |
| T381V | 0.89 |
| R383A | 0.51 |
| R383E | 0.51 |
| R383H | 0.71 |
| R383I | 0.71 |
| R383K | 1.30 |
| R383L | 1.31 |
| R383M | 0.61 |
| R383N | 0.77 |
| T381E | 0.35 |
| R383S | 0.87 |
| R383T | 0.98 |
| R383V | 1.05 |
| K385A | 1.12 |
| K385G | 0.62 |
| K385H | 0.50 |
| K385N | 0.41 |
| K385Q | 0.73 |
| K385R | 0.94 |
| K385S | 1.05 |
| K385T | 0.46 |
| K385V | 0.43 |
| T387S | 0.93 |
| L388F | 0.92 |
| L388H | 0.47 |
| L388I | 0.98 |
| L388M | 0.79 |
| L388R | 0.60 |
| L388T | 0.51 |
| L388V | 0.78 |
| L388W | 0.77 |
| L388Y | 1.18 |
| E392W | 0.31 |
| E389A | 1.14 |
| E389G | 0.91 |
| E389H | 1.17 |
| E389K | 1.91 |
| E389L | 0.65 |
| E389M | 0.60 |
| E389P | 0.75 |
| E389Q | 0.69 |
| E389R | 0.94 |
| E389S | 1.08 |
| E389T | 0.70 |
| E389Y | 0.77 |
| L391C | 0.90 |
| E392A | 0.58 |
| E392F | 0.54 |
| E392G | 1.00 |
| E392K | 0.66 |
| E392L | 0.80 |
| E392M | 1.54 |
| E392Q | 1.01 |
| E392R | 0.66 |
| E392S | 0.52 |
| E392T | 0.72 |
| E392V | 1.27 |
| E392Y | 0.92 |
| Q393A | 1.26 |
| Q393D | 0.45 |
| Q393F | 1.23 |
| Q393H | 1.05 |
| Q393K | 0.80 |
| Q393L | 0.91 |
| Q393M | 0.80 |
| Q393N | 0.72 |
| Q393R | 0.74 |
| Q393S | 1.15 |
| Q393T | 0.41 |
| F394L | 0.56 |
| F394W | 0.41 |
| S395A | 1.10 |
| S395G | 0.77 |
| S395H | 0.56 |
| S395K | 0.96 |
| S395R | 1.98 |
| E396A | 0.52 |
| E396D | 0.64 |
| E396H | 0.47 |
| E396Q | 0.73 |
| E396R | 0.61 |
| E396S | 0.61 |
| E396T | 0.89 |
| E396L | 0.39 |
| Y399A | 1.01 |
| Y399C | 0.46 |
| Y399E | 1.49 |
| S407L | 0.4 |
| Y399K | 1.94 |
| Y399M | 2.70 |
| Y399N | 0.52 |
| Y399Q | 1.18 |
| Y399R | 1.20 |
| Y399S | 1.01 |
| Y399T | 2.40 |
| Y399V | 1.44 |
| Y399W | 1.92 |
| S401A | 0.82 |
| S401E | 0.46 |
| S401N | 0.42 |
| Y403F | 0.62 |
| S404A | 0.63 |
| S404P | 0.64 |
| S401G | 0.38 |
| T405F | 0.36 |
| T405A | 0.56 |
| T405G | 2.32 |
| T405K | 0.74 |
| T405M | 0.48 |
| T405P | 0.64 |
| T405Q | 0.75 |
| T405R | 0.60 |
| T405S | 0.94 |
| T405W | 0.73 |
| T405Y | 0.44 |
| L406A | 0.70 |
| L406C | 0.98 |
| L406E | 0.73 |
| L406F | 1.42 |
| L406G | 1.00 |
| L406I | 0.61 |
| L406N | 0.76 |
| L406Q | 0.93 |
| L406S | 0.47 |
| L406T | 0.83 |
| L406V | 0.87 |
| L406Y | 0.74 |
| S407A | 1.16 |
| S407D | 1.52 |
| S407E | 1.38 |
| S407F | 1.42 |
| S407G | 0.75 |
| S407H | 1.34 |
| S407M | 0.74 |
| K411H | 0.33 |
| S407N | 0.72 |
| S407P | 0.94 |
| S407Q | 1.71 |
| S407R | 1.04 |
| S407V | 0.56 |
| S407W | 0.41 |
| K409A | 2.18 |
| K409D | 0.65 |
| K409E | 0.62 |
| K409G | 0.50 |
| K409H | 0.64 |
| K409I | 0.51 |
| K409P | 0.48 |
| K409Q | 3.33 |
| K409R | 0.84 |
| K409S | 0.72 |
| I083K | 0.30 |
| K409T | 0.63 |
| K409V | 0.48 |
| A412Y | 0.66 |
| E410D | 0.47 |
| E410K | 0.70 |
| E410M | 0.42 |
| E410N | 0.67 |
| E410P | 0.73 |
| E410Q | 0.85 |
| E410R | 0.61 |
| E410S | 0.81 |
| E410T | 1.54 |
| E410V | 0.65 |
| E410Y | 0.62 |
| K411A | 0.48 |
| K411N | 1.02 |
| K411P | 0.42 |
| K411R | 0.97 |
| K411S | 1.21 |
| K411T | 0.63 |
| K411V | 0.99 |
| A412D | 0.74 |
| A412G | 0.80 |
| A412I | 0.81 |
| E220M | 0.36 |
| P226W | 0.51 |
| A412L | 0.65 |
| A412N | 0.86 |
| A412P | 0.77 |
| A412R | 0.66 |
| A412S | 0.86 |
| A412V | 0.53 |
| A412W | 0.54 |
| D413E | 0.52 |
| D413K | 0.42 |
| D413N | 0.94 |
| D413R | 0.50 |
| D413T | 0.41 |
| V414I | 1.12 |
| V414M | 0.53 |
| K415G | 0.40 |
| K415S | 0.42 |
| K415W | 0.42 |
| D416F | 0.41 |
| D416G | 0.67 |
| D416H | 0.57 |
| D416I | 0.63 |
| D416K | 0.76 |
| D416L | 0.75 |
| D416N | 0.73 |
| D416Q | 0.83 |
| D416R | 0.46 |
| V237C | 0.35 |
| D416T | 0.85 |
| D416V | 0.59 |
| D416Y | 0.40 |
| T417I | 1.22 |
| D413A | 0.38 |
| D413S | 0.39 |
| K415Y | 0.39 |
| D418A | 0.92 |
| D418E | 1.31 |
| D418F | 0.81 |
| L089P | 0.38 |
| D418G | 0.45 |
| D418I | 0.99 |
| D418L | 1.28 |
| D418M | 1.09 |
| D418N | 0.91 |
| D418P | 2.11 |
| D418Q | 1.05 |
| D418R | 1.18 |
| D418S | 0.78 |
| D418V | 1.43 |
| D418Y | 0.97 |
| A419E | 0.45 |
| A419F | 2.17 |
| A419G | 0.42 |
| A419H | 1.21 |
| A419I | 1.64 |
| A419K | 1.88 |
| A419L | 0.56 |
| A419N | 0.53 |
| V421I | 0.39 |
| A419R | 1.81 |
| A419S | 2.65 |
| A419W | 0.69 |
| A419Y | 1.44 |
| V420I | 1.04 |
| V420P | 0.48 |
| D421A | 1.28 |
| D421E | 0.81 |
| D421G | 0.62 |
| D421H | 1.98 |
| K255G | 0.39 |
| D421K | 2.42 |
| D421L | 0.73 |
| D421M | 0.94 |
| D421N | 1.89 |
| D421Q | 1.54 |
| D421R | 2.21 |
| D421S | 2.12 |
| K094C | 0.33 |
| D421T | 0.80 |
| D421Y | 0.66 |
| V422I | 0.42 |
| V422T | 0.49 |
| A425G | 1.20 |
| A425I | 0.44 |
| A425K | 1.75 |
| A425M | 0.70 |
| A425N | 0.46 |
| A425R | 0.49 |
| A425S | 0.47 |
| D426E | 0.62 |
| D426G | 0.85 |
| F433L | 1.87 |
| F433P | 0.95 |
| F433R | 1.63 |
| F433S | 0.86 |
| F433T | 1.86 |
| F433V | 1.63 |
| F433W | 1.28 |
| L434F | 0.41 |
| L434G | 0.47 |
| L263H | 0.36 |
| L434I | 0.89 |
| L434M | 0.60 |
| L434V | 0.46 |
| K435A | 1.08 |
| K435C | 0.53 |
| K435E | 0.78 |
| K435G | 0.64 |
| K435H | 1.05 |
| K435R | 1.01 |
| K435S | 1.03 |
| K435T | 0.73 |
| K435V | 0.44 |
| K435Y | 0.50 |
| P436D | 1.19 |
| P436E | 0.74 |
| P436G | 1.19 |
| L105H | 0.36 |
| P436H | 0.72 |
| V272M | 0.31 |
| P436I | 0.84 |
| P436K | 2.05 |
| P436L | 0.63 |
| P436M | 0.61 |
| P436Q | 0.86 |
| P436R | 1.00 |
| P436S | 0.92 |
| P436T | 0.59 |
| P436W | 0.43 |
| P436Y | 0.49 |
| P437A | 0.56 |
| P437D | 0.62 |
| P437G | 0.50 |
| P437H | 1.11 |
| P437I | 2.46 |
| P437K | 0.83 |
| P437L | 0.51 |
| P437M | 2.55 |
| P437Q | 0.96 |
| D275L | 0.24 |
| P437R | 0.85 |
| P437S | 0.57 |
| P437Y | 0.42 |
| M438A | 0.75 |
| M438C | 0.63 |
| M438D | 0.87 |
| M438E | 0.72 |
| M438G | 0.83 |
| M438L | 0.86 |
| M438N | 1.08 |
| M438P | 0.81 |
| M438Q | 0.85 |
| M438R | 0.99 |
| M438S | 0.83 |
| M438T | 3.99 |
| M438V | 0.85 |
| P125A | 0.36 |
| M438W | 0.57 |
| E439A | 1.20 |
| E439C | 0.58 |
| E439F | 1.00 |
| E439G | 1.22 |
| E439H | 0.74 |
| E439K | 1.20 |
| E439L | 0.88 |
| E439P | 1.16 |
| Q276G | 0.36 |
| E439Q | 1.32 |
| E439S | 1.02 |
| E439T | 1.15 |
| E439V | 1.57 |
| E439W | 0.62 |
| T440A | 1.22 |
| T440D | 1.03 |
| T440E | 1.00 |
| T440F | 0.85 |
| T440G | 0.86 |
| T440H | 3.00 |
| T440I | 1.04 |
| T440L | 0.97 |
| T440M | 1.08 |
| T440P | 0.88 |
| T440R | 1.77 |
| T440S | 1.17 |
| T440V | 1.02 |
| T440Y | 1.11 |
| E441A | 1.47 |
| E441D | 0.67 |
| E441F | 3.91 |
| E441G | 0.87 |
| E441H | 0.65 |
| E441K | 0.80 |
| E441L | 0.82 |
| E441N | 0.82 |
| E441Q | 0.81 |
| E441S | 0.79 |
| E441T | 0.66 |
| E441V | 0.54 |
| E441Y | 0.51 |
| E442C | 1.38 |
| E442G | 0.51 |
| E442H | 0.76 |
| E442K | 0.73 |
| E442P | 0.91 |
| E442Q | 0.74 |
| D284Y | 0.37 |
| L286W | 0.38 |
| E442R | 3.94 |
| E442T | 0.61 |
| E442V | 0.65 |
| E442Y | 0.60 |
| P443A | 1.63 |
| P443E | 1.07 |
| P443F | 0.70 |
| P443G | 1.12 |
| P443H | 1.08 |
| P443L | 1.19 |
| P443M | 1.99 |
| P443N | 1.25 |
| P443Q | 0.96 |
| P443R | 1.04 |
| P443S | 0.99 |
| P443T | 0.87 |
| P443W | 0.64 |
| Q444M | 0.37 |
| Q444D | 0.97 |
| Q444E | 1.19 |
| Q444F | 0.66 |
| Q444G | 0.93 |
| Q444H | 0.97 |
| Q444I | 0.58 |
| Q444K | 1.03 |
| Q444N | 1.01 |
| Q444R | 0.85 |
| Q444V | 1.12 |
| Q444W | 0.64 |
| Q444Y | 0.67 |
| I445A | 0.97 |
| I445G | 0.98 |
| I445H | 1.35 |
| I445L | 1.06 |
| I445M | 1.57 |
| I445N | 1.24 |
| I445P | 1.67 |
| I445Q | 1.26 |
| I445R | 1.08 |
| I445S | 1.21 |
| I445T | 1.38 |
| I445V | 1.25 |
| I445W | 0.69 |
| I445Y | 0.53 |
| F446A | 1.58 |
| F446C | 0.75 |
| F446D | 1.18 |
| F446E | 1.10 |
| F446G | 1.12 |
| F446H | 1.28 |
| F446I | 1.06 |
| F446K | 0.94 |
| F446L | 0.93 |
| F446M | 1.31 |
| F446Q | 0.72 |
| F446R | 0.89 |
| F446T | 0.89 |
| F446V | 0.91 |
| F446W | 1.40 |
| Y447D | 3.25 |
| Y447E | 1.36 |
| Y447F | 1.41 |
| Y447G | 0.92 |
| Y447I | 1.36 |
| Y447L | 1.09 |
| Y447M | 0.90 |
| Y447N | 1.58 |
| Y447P | 1.46 |
| Y447Q | 2.37 |
| Y447R | 1.12 |
| Y447T | 1.90 |
| Y447V | 1.38 |
| Y447W | 1.07 |

2. Inactive Mutants

The other mutants that exhibited less than 20% hyaluronidase activity of wildtype PH20, in at least one of the duplicates, were rescreened to confirm that the dead mutants were inactive. To confirm the inactive mutants, the hyaluronidase activity assay described in Example 3 was modified to incorporate an overnight 37° C. substrate-sample incubation step prior to measurement of enzymatic activity. The modified assay was intended to detect PH20 activities below 0.2 U/mL.

The preparation of the bHA coated plates and blocking of the plates prior to addition of the transfected variant supernatants or wildtype PH20 was the same as described in Example 3. The assay was modified as follows. First, transfected variant supernatants or wildypte PH20 not containing a mutation generated as described in Example 2 were diluted in duplicate 1:25 in assay diluent. For the standard curve, 1:3 serial dilutions of rHuPH20 (generated as described in Example 1) were made in assay diluent in duplicate starting from 0.1 U/mL down to 0.00014 U/mL. A blank well also was included. Then, 100 μl of the diluted samples or standard were added to pre-designated wells of the bHA-coated and blocked plate and allowed to incubate at 37° C. overnight. After the incubation, the plates were washed and binding to bHA detected as described above in Example 3. Optical density was measured at 450 nm within 30 minutes of adding the stop solution.

The identified reconfirmed inactive mutants are set forth in Table 8. The Table sets forth the amino acid replacement compared to the sequence of amino acids of PH20 set forth in SEQ ID NO:3.

TABLE 8
Inactive Mutants

| | | | | | | | |
|---|---|---|---|---|---|---|---|
| N002H | R060V | R121W | C189P | P236I | V287N | L336W | G377V |
| N002K | R060Y | R121Y | C189R | P236L | V287P | L336Y | G378D |
| N002W | L061A | N122A | C189S | P236N | V287Q | A337C | G378E |
| N002Y | L061E | N122C | C189T | P236Q | V287R | A337F | G378F |
| F003A | L061F | N122E | C189V | P236T | V287S | A337G | G378I |
| F003G | L061G | N122F | C189W | P236Y | Y288D | A337I | G378L |
| F003K | L061H | N122I | C189Y | A238F | Y288E | A337K | G378M |
| F003P | L061N | N122K | Y190C | A238G | Y288F | A337L | G378Q |
| F003T | L061P | N122Q | Y190E | A238L | Y288G | A337M | G378T |
| F003V | L061Q | N122R | Y190F | A238P | Y288H | A337R | G378W |
| R004D | L061R | N122S | Y190G | A238V | Y288I | A337T | G378Y |
| R004E | L061T | N122T | Y190H | A238W | Y288K | A337W | K379A |
| R004F | L061W | N122V | Y190K | A238Y | Y288P | A338C | K379C |
| R004G | L061Y | W123A | Y190L | A239C | Y288R | A338D | K379E |
| R004L | G062A | W123C | Y190N | A239F | Y288T | A338E | K379F |
| R004P | G062C | W123D | Y190Q | A239G | T289A | A338F | K379I |
| R004W | G062D | W123E | Y190R | A239H | T289C | A338G | K379L |
| R004Y | G062F | W123H | Y190S | A239I | T289E | A338H | K379M |
| A005D | G062I | W123L | Y190T | A239L | T289G | A338I | K379W |
| A005G | G062K | W123M | Y190V | A239P | T289H | A338K | F380C |
| A005I | G062L | W123P | Y190W | A239R | T289L | A338L | F380D |
| A005L | G062M | W123Q | N191A | A239S | T289P | A338P | F380E |
| A005M | G062P | W123R | N191E | A239T | T289Q | A338R | F380G |
| A005N | G062Q | W123S | N191F | A239V | T289R | A338T | F380Q |
| A005P | G062R | W123T | N191G | A239W | T289S | A338V | F380R |
| A005Q | G062S | W123V | N191K | A239Y | T289Y | K339D | F380S |
| A005R | G062T | W123Y | N191L | T240E | F290D | K339E | T381G |
| A005T | G062V | K124C | N191M | T240F | F290Q | K339F | T381L |
| A005V | G062Y | K124D | N191P | T240G | F290Y | K339G | T381P |
| A005W | Y063C | K124E | N191Q | T240N | G291A | K339H | T381W |
| A005Y | Y063G | K124F | N191R | T240W | G291C | K339L | T381Y |
| P006E | Y063P | K124N | N191S | T240Y | G291D | K339N | V382E |
| P006F | Y064A | P125C | N191T | L241A | G291E | K339P | V382G |
| P006T | Y064C | P125D | N191V | L241C | G291F | K339S | V382H |
| P006V | Y064D | P125G | N191W | L241D | G291M | K339T | V382K |
| P006Y | Y064E | P125L | N191Y | L241E | G291N | K339V | V382L |
| P007C | Y064F | P125N | H192C | L241G | G291T | K339W | V382M |
| P007D | Y064G | P125W | H192F | L241I | G291W | K339Y | V382N |
| P007F | Y064H | K126F | H192G | L241P | G291Y | M340A | V382P |
| P007G | Y064I | K126H | H192K | L241R | E292I | M340C | V382Q |
| P007H | Y064K | K126I | H192L | L241S | E292L | M340D | V382R |
| P007I | Y064L | K126L | H192M | L241T | E292T | M340E | V382S |
| P007K | Y064P | K126N | H192N | L241V | T293E | M340F | V382T |
| P007L | Y064Q | K126P | H192P | L241W | T293N | M340G | V382W |
| P007Q | Y064R | K126Y | H192Q | Y242A | V294A | M340H | V382Y |
| P007R | Y064S | D127K | H192R | Y242C | V294E | M340K | R383G |
| P007S | Y064T | V128E | H192V | Y242D | V294G | M340P | R383P |
| P007T | Y064V | V128P | H192W | Y242G | V294H | M340R | G384C |
| P007W | Y064W | Y129A | H192Y | Y242I | V294K | M340S | G384F |
| P007Y | P065A | Y129C | H193A | Y242L | V294L | M340T | G384M |
| V008D | P065C | Y129D | H193D | Y242M | V294N | M340V | G384Q |
| V008E | P065D | Y129E | H193K | Y242P | V294P | M340W | G384S |
| V008G | P065G | Y129G | H193L | Y242R | V294Q | C341A | G384T |
| V008H | P065H | Y129H | H193M | Y242S | V294R | C341E | K385C |
| V008N | P065I | Y129L | H193P | Y242T | V294S | C341G | K385L |
| V008R | P065K | Y129P | H193V | Y242V | V294T | C341H | K385M |
| V008S | P065N | Y129Q | Y194A | Y242W | V294W | C341K | K385P |
| V008W | P065R | Y129S | Y194C | V243C | A295C | C341L | K385W |
| I009C | P065S | Y129T | Y194I | V243D | A295G | C341M | K385Y |
| I009D | P065T | Y129V | Y194L | V243F | A295H | C341N | P386A |
| I009E | P065V | Y129W | Y194P | V243G | A295I | C341Q | P386C |
| I009G | P065W | K130C | Y194S | V243H | A295L | C341R | P386F |
| I009N | P065Y | K130D | Y194T | V243L | A295N | C341S | P386G |
| I009P | Y066A | K130G | Y194V | V243M | A295P | C341T | P386H |
| P010F | Y066C | K130H | K195S | V243P | A295T | C341V | P386I |
| P010I | Y066D | K130L | P197C | V243Q | A295V | C341Y | P386L |
| P010L | Y066E | K130N | G198V | V243R | A295Y | S342D | P386M |
| P010M | Y066G | K130S | G198W | V243S | L296C | S342E | P386N |
| P010Y | Y066I | K130T | Y199E | V243W | L296F | S342F | P386Q |
| N011A | Y066K | K130W | Y199G | V243Y | L296G | S342H | P386R |
| N011C | Y066L | K130Y | Y199H | R244A | L296I | S342K | P386S |
| N011F | Y066N | N131P | Y199I | R244D | L296K | S342L | P386T |
| N011I | Y066P | R132P | Y199K | R244G | L296M | S342M | P386V |
| N011L | Y066S | S133D | Y199L | R244I | L296Q | S342P | P386Y |
| N011P | Y066T | S133E | Y199P | R244V | L296R | S342Q | T387C |
| N011T | Y066V | S133F | Y199R | R244Y | L296S | S342R | T387E |
| N011W | I067D | S133G | Y199S | N245A | L296T | S342T | T387F |
| N011Y | I067E | S133H | Y199W | N245C | L296V | S342Y | T387G |
| V012G | I067G | S133L | N200A | N245F | L296W | Q343C | T387H |
| V012H | I067P | S133M | N200F | N245L | L296Y | Q343D | T387I |
| V012W | I067R | S133N | N200G | N245P | G297C | Q343F | T387L |
| P013E | I067T | S133P | N200H | N245Q | G297E | Q343I | T387M |
| P013G | I067W | S133R | N200K | N245R | G297H | Q343P | T387N |
| P013I | D068A | S133T | N200L | N245S | G297L | Q343W | T387V |
| P013L | D068C | S133V | N200M | N245T | G297N | V344F | T387W |
| P013M | D068G | S133W | N200P | N245V | G297P | V344G | T387Y |
| P013V | D068I | I134A | N200Q | R246A | G297Q | V344H | L388C |
| F014A | D068L | I134C | N200R | R246C | G297R | V344L | L388G |
| F014E | D068P | I134D | N200S | R246D | G297S | V344M | L388P |
| F014G | D068V | I134F | N200W | R246E | G297T | V344N | L388Q |
| F014H | D068Y | I134G | N200Y | R246G | G297Y | V344P | L388S |
| F014K | S069N | I134H | G201A | R246H | A298C | V344Q | E389F |
| F014N | S069T | I134K | G201F | R246I | A298E | V344R | E389V |
| F014P | I070Q | I134P | G201L | R246K | A298L | V344S | D390A |
| F014Q | T071P | I134Q | G201M | R246L | A298M | V344T | D390C |
| F014W | G072C | I134R | G201N | R246M | A298N | V344W | D390E |
| L015E | G072F | I134S | G201P | R246P | A298P | V344Y | D390F |
| L015F | G072H | I134W | G201R | R246S | A298Q | L345A | D390G |
| L015G | G072I | E135P | G201S | R246T | A298S | L345C | D390H |
| L015K | G072P | L136P | G201T | R246V | A298T | L345E | D390L |
| L015N | G072V | V137F | G201V | R246W | A298W | L345H | D390N |
| L015P | G072W | V137G | G201W | V247A | A298Y | L345K | D390P |
| L015Q | V073P | V137H | S202A | V247C | S299A | L345N | D390R |
| L015R | V075D | V137N | S202E | V247F | S299C | L345Q | D390S |
| L015S | V075G | V137P | S202F | V247H | S299D | L345R | D390T |
| L015Y | V075P | V137R | S202G | V247N | S299F | L345T | D390V |
| W016A | N076A | V137W | S202H | V247P | S299G | L345V | D390W |
| W016C | N076C | V137Y | S202K | V247Q | S299H | L345Y | D390Y |
| W016D | N076F | Q138V | S202N | V247R | S299L | C346A | L391A |
| W016E | N076G | Q139P | S202P | V247S | S299M | C346D | L391D |
| W016F | N076I | Q143C | S202Q | V247T | S299P | C346F | L391G |
| W016G | N076K | Q143H | S202R | V247W | S299Q | C346G | L391H |
| W016H | N076L | Q143P | S202V | V247Y | S299T | C346I | L391K |
| W016K | N076P | Q143R | S202W | R248C | G300A | C346K | L391N |
| W016M | N076Q | Q143S | S202Y | R248D | G300C | C346L | L391P |
| W016P | N076R | Q143T | C203A | R248E | G300D | C346M | L391Q |
| W016R | N076S | L144A | C203D | R248G | G300E | C346P | L391R |
| W016S | N076T | L144E | C203E | R248I | G300F | C346R | L391S |
| W016T | N076V | L144F | C203G | R248M | G300L | C346S | L391T |
| W016Y | N076W | L144I | C203H | R248P | G300M | C346T | L391V |
| A017D | G077D | L144K | C203L | R248T | G300N | C346V | L391W |
| A017E | G077E | L144P | C203M | E249A | G300P | C346W | L391Y |
| A017G | G077L | L144Q | C203N | E249G | G300Q | Q347C | E392C |
| A017H | G077P | L144S | C203Q | E249H | G300S | Q347F | E392P |
| A017I | G077Q | L144V | C203R | E249I | G300T | Q347I | Q393C |
| A017L | G077R | L144Y | C203S | E249K | G300V | Q347P | Q393P |
| A017N | G077T | S145T | C203T | E249M | G300W | Q347T | F394A |
| A017P | G077V | S145W | C203V | E249Q | I301E | Q347V | F394D |
| A017Q | G078A | A149E | F204A | E249S | I301G | Q347W | F394E |
| A017R | G078D | A149P | F204C | E249Y | I301H | E348C | F394G |
| A017S | G078I | T150V | F204E | A250C | I301K | E348H | F394I |
| A017T | G078M | K152L | F204G | A250F | I301M | E348I | F394K |
| A017V | G078P | A153E | F204H | A250G | I301N | E348L | F394N |
| A017W | G078T | A153F | F204I | A250H | I301P | E348P | F394P |
| A017Y | G078Y | A153M | F204K | A250K | I301Q | E348Q | F394Q |
| W018C | I079A | A153P | F204Q | A250L | I301R | E348R | F394R |
| W018D | I079D | A153R | F204R | A250M | I301S | E348T | F394S |
| W018F | I079F | A153T | F204S | A250N | I301W | E348V | F394T |
| W018G | I079G | A153V | F204T | A250P | I301Y | E348W | F394V |
| W018H | I079H | K154D | V206C | A250Q | V302C | E348Y | S395C |
| W018I | I079K | K154E | V206D | A250R | V302D | Q349D | S395L |
| W018L | I079N | K154G | V206F | A250S | V302E | Q349F | S395M |
| W018M | I079P | K154P | V206G | A250T | V302F | Q349G | S395P |
| W018P | I079S | K154S | V206P | A250V | V302G | Q349P | E396C |
| W018Q | I079W | K154W | V206Y | A250W | V302H | Q349V | E396F |
| W018S | I079Y | K154Y | E207A | I251D | V302L | Q349W | E396G |
| W018T | P080A | Q155P | E207F | I251F | V302M | Q349Y | E396I |
| W018V | P080D | Q155Y | E207G | I251G | V302P | G350A | E396P |
| W018Y | P080E | E156P | E207M | I251H | V302R | G350D | E396Y |
| N019A | P080F | F157A | E207P | I251K | V302S | G350E | K397A |
| N019C | P080G | F157C | E207Q | I251P | V302T | G350F | K397C |
| N019F | P080I | F157D | E207R | I251S | V302Y | G350H | K397E |
| N019G | P080K | F157E | E207S | I251T | I303A | G350K | K397F |
| N019H | P080L | F157G | E207T | I251W | I303C | G350L | K397G |
| N019I | P080M | F157H | E207V | R252A | I303D | G350M | K397I |
| N019L | P080N | F157I | E207W | R252D | I303E | G350N | K397L |
| N019M | P080R | F157K | I208D | R252E | I303F | G350P | K397M |
| N019P | P080S | F157L | I208G | R252F | I303G | G350R | K397P |
| N019Q | P080T | F157M | I208P | R252G | I303K | G350S | K397Q |
| N019R | P080V | F157P | I208W | R252H | I303L | G350T | K397T |
| N019S | P080Y | F157Q | K209C | R252I | I303M | G350V | K397V |
| N019V | Q081A | F157R | K209P | R252K | I303R | G350Y | F398A |
| N019W | Q081C | F157S | R210A | R252L | I303W | V351C | F398C |
| N019Y | Q081E | F157T | R210C | R252N | I303Y | V351D | F398E |
| A020D | Q081G | F157V | R210D | R252P | W304A | V351E | F398G |
| A020E | Q081H | E158D | R210E | R252S | W304C | V351F | F398H |
| A020F | Q081L | E158K | R210G | R252T | W304D | V351H | F398I |
| A020H | Q081N | E158P | R210K | R252Y | W304G | V351N | F398L |
| A020K | Q081P | E158R | R210M | V253A | W304I | V351R | F398N |
| A020L | Q081S | E158Y | R210N | V253D | W304M | V351W | F398P |
| A020N | Q081V | K159W | R210P | V253E | W304N | V351Y | F398R |
| A020P | Q081W | K159Y | R210S | V253G | W304P | C352A | F398S |
| A020R | Q081Y | G161W | R210T | V253H | W304Q | C352D | F398T |
| A020T | K082W | D163C | R210V | V253L | W304S | C352E | F398V |
| A020V | K082Y | D163P | R210W | V253M | W304T | C352F | F398W |
| A020Y | I083E | F164A | R210Y | V253N | W304V | C352G | F398Y |
| P021A | I083K | F164C | N211C | V253Q | W304Y | C352K | Y399D |
| P021C | S084Y | F164D | N211F | V253R | G305L | C352M | Y399P |
| P021D | L085A | F164E | N211G | V253S | G305P | C352P | C400A |
| P021E | L085C | F164G | N211H | V253W | G305Q | C352Q | C400D |
| P021G | L085D | F164H | N211I | S254C | G305R | C352R | C400E |
| P021H | L085E | F164N | N211K | S254D | G305S | C352S | C400F |
| P021I | L085F | F164P | N211M | S254E | G305T | C352T | C400G |
| P021L | L085G | F164Q | N211P | S254G | G305V | C352V | C400I |
| P021M | L085H | F164R | N211R | S254I | G305Y | C352W | C400L |
| P021R | L085N | L165C | N211S | S254K | T306A | C352Y | C400M |
| P021S | L085Q | L165H | N211T | S254L | T306C | I353C | C400P |
| P021T | L085S | L165P | N211V | S254P | T306H | I353F | C400Q |
| P021V | L085T | L165T | N211W | S254Q | T306I | I353G | C400R |
| P021W | Q086C | V166D | D212A | S254R | T306L | I353H | C400S |
| S022C | Q086P | E167V | D212G | S254T | T306V | I353K | C400T |
| S022E | D087P | T168A | D212H | S254V | T306W | I353L | C400V |
| S022G | H088A | T168C | D212I | S254W | T306Y | I353M | C400Y |
| S022K | H088C | T168D | D212K | S254Y | L307C | I353Q | S401C |
| S022P | H088E | T168E | D212L | K255C | | I353R | S401F |
| E023A | H088F | T168F | D212M | K255D | L307I | I353S | S401H |
| E023F | H088G | T168G | D212P | K255L | L307P | I353W | S401K |
| E023L | H088I | T168K | D212V | K255P | S308C | R354C | S401R |
| E023M | H088K | T168L | D212W | K255V | S308F | R354D | S401W |
| E023N | H088L | T168P | D213P | K255W | S308L | R354E | S401Y |
| E023P | H088M | T168R | D213S | I256C | S308M | R354G | C402A |
| E023R | H088P | T168S | L214A | I256D | S308V | R354H | C402D |
| E023S | H088R | T168V | L214C | I256E | S308W | R354I | C402E |
| E023T | H088S | T168W | L214D | I256G | S308Y | R354K | C402F |
| E023V | H088T | T168Y | L214E | I256P | M310C | R354L | C402L |
| C025D | H088V | I169A | L214G | P257D | M310E | R354M | C402M |
| C025E | H088Y | I169D | L214H | D258L | M310F | R354P | C402P |
| C025F | L089A | I169F | L214K | D258P | M310K | R354Q | C402Q |
| C025G | L089D | I169G | L214N | D258V | M310L | R354S | C402R |
| C025H | L089E | I169H | L214P | D258W | R311C | R354V | C402S |
| C025I | L089G | I169K | L214R | K260C | R311E | R354W | C402T |
| C025K | L089Q | I169N | L214S | K260P | R311F | R354Y | C402V |
| C025L | L089S | I169P | L214T | S261P | R311I | K355D | C402W |
| C025N | L089T | I169Q | L214Y | P262A | R311L | K355F | C402Y |
| C025P | L089W | I169S | S215C | P262D | R311P | K355G | Y403A |
| C025R | L089Y | I169T | S215P | P262E | R311V | K355H | Y403C |
| C025S | D090C | I169Y | W216D | P262F | R311W | K355L | Y403E |
| C025T | D090G | K170C | W216E | P262G | S312C | K355M | Y403G |
| C025V | K091D | K170D | W216G | P262H | S312E | K355N | Y403H |
| C025Y | K091E | K170E | W216H | P262I | S312M | K355P | Y403K |
| G027C | K091F | K170G | W216I | P262K | S312V | K355Q | Y403L |
| L033C | K091G | K170M | W216K | P262Q | S312W | K355R | Y403M |
| L033D | K091H | K170P | W216L | P262R | M313C | K355S | Y403N |
| L033H | K091I | K170W | W216M | P262S | K314C | K355T | Y403P |
| L033N | K091L | K170Y | W216N | P262T | K314L | K355V | Y403Q |
| L033V | K091N | L171C | W216P | P262V | K314W | K355W | Y403R |
| L033Y | K091T | L171D | W216Q | P262W | S315C | K355Y | Y403T |
| D034I | A092E | L171H | W216R | P262Y | S315I | N356C | S404C |
| D034L | A092F | L171M | W216T | L263E | S315V | N356G | S404D |
| D034N | A092H | L171N | W216V | L263F | C316E | N356K | S404F |
| D034S | A092K | L171R | L217A | L263P | C316G | N356L | S404G |
| D034T | A092P | L171S | L217C | L263Q | C316I | N356P | S404H |
| D034V | A092Q | L171W | L217G | L263W | C316K | N356R | S404L |
| M035A | A092R | L171Y | L217H | P264D | C316L | N356T | S404M |
| M035D | A092W | G172D | L217P | P264E | C316M | N356V | S404N |
| M035G | A092Y | G172E | L217Q | P264F | C316P | N356W | S404R |
| M035P | K094G | G172I | L217S | P264G | C316R | W357D | S404V |
| M035R | K094P | G172L | L217T | P264L | C316S | W357E | S404W |
| M035S | D095A | G172P | L217V | P264M | C316T | W357F | S404Y |
| S036C | D095C | G172Q | L217W | P264R | C316V | W357G | T405C |
| S036F | D095E | G172T | W218A | P264T | C316W | W357L | T405I |
| S036V | D095F | G172V | W218I | P264V | C316Y | W357M | T405V |
| S036W | D095G | G172W | W218K | P264W | L317G | W357Q | L406P |
| S036Y | D095H | G172Y | W218L | P264Y | L317P | W357R | L406R |
| L037C | D095K | K173D | W218P | V265A | L318C | N358E | C408A |
| L037E | D095L | K173E | W218S | V265D | L318P | N358H | C408E |
| L037G | D095M | K173G | W218V | V265F | L318W | N358I | C408F |
| L037N | D095P | K173H | N219P | V265G | L319C | N358K | C408G |
| L037S | D095Q | K173I | E220G | V265H | L319E | N358P | C408I |
| F038E | D095S | K173L | E220K | V265K | L319F | N358Q | C408K |
| F038G | D095V | K173M | E220N | V265L | L319G | N358R | C408L |
| F038K | D095W | K173P | E220P | V265M | L319H | N358W | C408P |
| F038L | D095Y | K173S | E220R | V265N | L319I | S359A | C408R |
| F038N | I096A | K173V | E220W | V265Q | L319K | S359F | C408S |
| F038Q | I096C | K173W | S221D | V265R | L319M | S359G | C408T |
| F038R | I096G | K173Y | S221E | V265S | L319P | S359L | C408V |
| F038T | I096H | L174P | S221H | F266A | L319Q | S359P | C408W |
| F038W | I096P | L175C | S221K | F266C | L319R | S359W | C408Y |
| S039C | I096R | L175D | S221P | F266G | L319S | S360A | E410W |
| S039D | I096S | L175G | S221R | F266H | L319V | S360C | K411D |
| S039F | I096T | L175K | T222P | F266M | L319W | S360E | K411E |
| S039W | I096W | L175P | T222Y | F266P | L319Y | S360F | K411F |
| F040A | F098P | L175R | A223C | F266Q | D320C | S360G | K411G |
| F040D | Y099C | L175S | A223D | F266R | D320P | S360I | A412E |
| F040E | Y099E | R176A | A223E | F266S | D320V | S360K | A412H |
| F040G | Y099G | R176C | A223G | F266T | N321E | S360L | D413H |
| F040K | Y099I | R176E | A223H | F266V | N321M | S360M | D413I |
| F040N | Y099N | R176F | A223K | F266W | N321P | S360P | D413K |
| F040R | Y099P | R176G | A223L | A267D | Y322C | S360Q | D413L |
| F040S | Y099V | R176H | A223P | A267G | Y322D | S360R | D413P |
| F040T | Y099W | R176I | A223Q | A267H | Y322E | S360V | V414A |
| F040V | M100C | R176P | A223R | A267I | Y322G | D361A | V414D |
| I041Q | M100E | R176Q | A223S | A267K | Y322I | D361C | V414E |
| G042D | M100F | R176S | A223T | A267N | Y322L | D361E | V414G |
| G042E | M100G | R176T | A223V | A267R | Y322N | D361G | V414H |
| G042H | M100N | R176V | A223W | A267S | Y322P | D361M | V414K |
| G042I | M100P | R176W | A223Y | A267W | Y322R | D361N | V414R |
| G042K | M100R | P177A | L224A | Y268A | Y322S | D361P | V414S |
| G042L | M100S | P177C | L224D | Y268C | Y322T | D361Q | V414T |
| G042M | M100T | P177D | L224E | Y268F | Y322V | D361R | K415C |
| G042P | M100W | P177F | L224F | Y268G | Y322W | D361S | K415D |
| G042Q | M100Y | P177G | L224G | Y268H | M323A | D361V | K415E |
| G042R | P101A | P177H | L224M | Y268K | M323C | D361W | K415P |
| G042S | P101C | P177L | L224P | Y268L | M323E | Y362A | D416C |
| G042T | P101F | P177M | L224Q | Y268N | M323G | Y362C | D416S |
| G042V | P101H | P177Q | L224R | Y268P | M323H | Y362E | T417A |
| S043A | P101I | P177R | L224S | Y268Q | M323K | Y362G | T417D |
| S043E | P101K | P177S | L224T | Y268S | M323N | Y362H | T417E |
| S043F | P101L | P177T | L224W | Y268T | M323R | Y362K | T417F |
| S043G | P101M | P177V | L224Y | Y268V | M323S | Y362L | T417G |
| S043I | P101N | P177W | Y225A | Y268W | M323T | Y362M | T417H |
| S043K | P101Q | N178E | Y225D | T269E | M323V | Y362N | T417K |
| S043L | P101R | N178I | Y225E | T269K | E324C | Y362P | T417M |
| S043Q | P101S | N178L | Y225G | T269L | E324F | Y362R | T417P |
| S043R | P101T | N178V | Y225H | T269M | E324P | Y362S | T417Q |
| S043V | V102P | N178W | Y225K | T269N | E324V | Y362T | T417R |
| P044A | D103A | N178Y | Y225P | T269P | E324W | Y362V | A419D |
| P044C | D103E | H179W | Y225Q | T269Q | E324Y | Y362W | A419P |
| P044F | D103F | L180A | Y225R | T269R | T325C | L363A | V420A |
| P044G | D103G | L180C | Y225T | R270A | T325R | L363C | V420D |
| P044H | D103H | L180E | Y225V | R270C | I326E | L363D | V420F |
| P044I | D103I | L180P | Y225W | R270E | I326G | L363E | V420G |
| P044L | D103L | L180R | P226A | R270F | I326H | L363F | V420H |
| P044N | D103Q | L180S | P226C | R270G | I326N | L363G | V420K |
| P044Q | D103R | W181A | P226D | R270H | I326W | L363H | V420L |
| P044R | D103T | W181C | P226E | R270I | L327A | L363I | V420N |
| P044S | D103V | W181D | P226F | R270P | L327E | L363P | V420R |
| P044T | D103W | W181E | P226G | R270Y | L327F | L363Q | V420S |
| P044W | D103Y | W181F | P226L | I271A | L327G | L363R | V420T |
| P044Y | N104F | W181H | P226N | I271D | L327H | L363S | V420W |
| R045A | N104P | W181I | P226Q | I271E | L327N | L363T | V420Y |
| R045D | N104W | W181K | P226R | I271H | L327Q | L363V | V422C |
| R045F | L105C | W181L | P226S | I271K | L327R | L363W | V422D |
| R045G | L105M | W181R | P226T | I271T | L327S | H364A | V422G |
| R045P | L105N | W181S | P226V | I271W | L327T | H364C | V422H |
| R045W | G106A | W181V | P226W | V272A | L327V | H364D | V422L |
| I046P | G106C | G182A | P226Y | V272H | L327W | H364E | V422M |
| I046W | G106D | G182C | S227A | V272L | L327Y | H364F | V422N |
| N047V | G106F | G182D | S227F | V272N | P329C | H364G | V422Q |
| A048P | G106H | G182E | S227G | V272P | P329F | H364K | V422R |
| T049C | G106L | G182H | S227H | V272W | P329G | H364L | V422S |
| T049D | G106M | G182N | S227I | F273A | P329H | H364M | V422Y |
| T049G | G106N | G182P | S227K | F273C | P329I | H364P | C423A |
| T049H | G106P | G182Q | S227L | F273D | P329K | H364R | C423D |
| T049P | G106S | G182R | S227M | F273G | P329L | H364S | C423E |
| | G106W | G182S | S227P | F273I | P329N | H364T | C423F |
| Q051C | G106Y | G182T | S227Q | F273L | P329Q | H364V | C423G |
| Q051F | M107A | G182V | S227R | F273P | P329R | H364Y | C423H |
| Q051I | M107C | G182Y | S227T | F273Q | P329S | L365A | C423L |
| Q051M | M107H | Y183C | S227V | F273S | P329T | L365C | C423M |
| Q051P | M107K | Y183D | S227W | F273V | P329V | L365D | C423P |
| Q051T | M107P | Y183E | S227Y | F273W | P329W | L365E | C423Q |
| Q051W | M107Q | Y183G | I228A | T274C | P329Y | L365G | C423R |
| Q051Y | M107S | Y183I | I228E | T274E | Y330A | L365M | C423S |
| G052C | M107V | Y183K | I228F | T274G | Y330C | L365N | C423T |
| G052E | M107W | Y183N | I228G | T274H | Y330D | L365P | C423V |
| G052F | A108D | Y183P | I228H | T274N | Y330E | L365Q | C423W |
| G052W | A108E | Y183Q | I228L | T274Q | Y330G | L365R | I424A |
| G052Y | A108F | Y183R | I228M | T274W | Y330I | L365S | I424C |
| V053A | A108K | Y183S | I228N | T274Y | Y330L | L365T | I424E |
| V053C | A108L | Y183V | I228P | D275A | Y330M | L365W | I424G |
| V053D | A108M | Y184A | I228R | D275F | Y330N | L365Y | I424H |
| V053E | A108P | Y184C | I228S | D275G | Y330P | N366A | I424N |
| V053G | A108Q | Y184D | I228T | D275I | Y330R | N366C | I424Q |
| V053H | A108T | Y184E | I228W | D275K | Y330S | N366E | I424R |
| V053L | A108V | Y184F | Y229E | D275L | Y330V | N366F | I424S |
| V053N | A108Y | Y184G | Y229F | D275M | Y330W | N366G | I424W |
| V053P | V109C | Y184H | Y229G | D275Q | I331A | N366K | I424Y |
| V053Q | V109D | Y184K | Y229K | D275T | I331C | N366M | A425E |
| V053R | V109E | Y184L | Y229L | D275V | I331D | N366P | A425L |
| V053S | V109L | Y184M | Y229P | D275W | I331E | N366Q | A425P |
| V053T | V109M | Y184P | Y229Q | Q276F | I331F | N366R | A425W |
| V053W | V109R | Y184R | Y229T | Q276P | I331H | N366T | A425Y |
| V053Y | V109T | Y184S | Y229V | Q276W | I331K | N366W | D426C |
| T054D | V109W | Y184V | Y229W | L278M | I331Q | P367E | D426F |
| T054E | I110F | L185A | L230A | L278P | I331R | P367F | D426M |
| T054G | I110K | L185D | L230E | K279A | I331S | P367I | D426R |
| T054P | I110L | L185E | L230G | K279C | I331T | P367L | G427A |
| T054R | I110M | L185F | L230H | K279F | I331W | P367M | G427C |
| T054Y | I110P | L185G | L230K | K279G | I331Y | P367Q | G427F |
| I055A | I110W | L185I | L230M | K279L | I332A | P367V | G427L |
| I055D | D111H | L185K | L230N | K279W | I332C | D368C | G427P |
| I055G | D111I | L185P | L230P | K279Y | I332D | D368P | |
| I055H | D111Q | L185R | L230R | F280D | I332E | D368W | G427V |
| I055N | W112C | L185S | L230S | F280I | I332F | N369C | G427W |
| I055P | W112E | L185T | L230T | F280L | I332G | N369E | G427Y |
| I055Q | W112G | L185V | L230V | F280M | I332H | N369F | V428A |
| I055R | W112H | L185W | L230W | F280N | I332K | N369I | V428C |
| I055T | W112L | L185Y | L230Y | F280R | I332L | N369K | V428D |
| I055V | W112N | F186A | N231A | F280S | I332N | N369L | V428E |
| I055Y | W112P | F186D | N231C | F280T | I332P | N369P | V428G |
| F056A | W112S | F186G | N231D | F280V | I332R | N369Q | V428H |
| F056C | E113R | F186H | N231F | F280W | I332S | N369V | V428N |
| F056E | E113V | F186I | N231G | L281A | I332T | N369W | V428R |
| F056G | E114I | F186K | N231H | L281D | I332Y | F370A | V428S |
| F056H | E114L | F186L | N231I | L281G | N333G | F370D | V428Y |
| F056I | E114P | F186N | N231K | L281H | N333H | F370E | C429A |
| F056K | E114T | F186P | N231L | L281I | N333I | F370G | C429D |
| F056L | E114V | F186Q | N231P | L281K | N333K | F370H | C429K |
| F056P | W115A | F186R | N231Q | L281N | N333P | F370K | C429L |
| F056R | W115C | F186S | N231R | L281P | N333R | F370L | C429N |
| F056S | W115D | F186V | N231S | L281Q | N333S | F370N | C429P |
| F056T | W115F | F186W | N231V | L281R | N333T | F370P | C429S |
| F056V | W115G | P187A | T232C | L281S | N333W | F370Q | C429T |
| F056W | W115H | P187F | T232G | L281V | N333Y | F370R | C429V |
| Y057A | W115I | P187G | T232H | L281W | V334A | F370S | C429W |
| Y057D | W115K | P187H | T232K | S282F | V334C | F370V | C429Y |
| Y057F | W115L | P187I | T232L | S282L | V334D | F370Y | I430A |
| Y057G | W115M | P187L | T232N | S282V | V334E | A371P | I430D |
| Y057I | W115R | P187M | T232P | S282W | V334G | A371W | I430E |
| Y057L | W115S | P187N | T232Q | S282Y | V334M | I372A | I430L |
| Y057M | W115V | P187Q | T232V | Q283A | V334N | I372D | I430M |
| Y057P | W115Y | P187R | T232Y | Q283C | V334R | I372E | I430N |
| Y057Q | R116A | P187S | Q233D | Q283D | V334S | I372F | I430S |
| Y057R | R116C | P187T | Q233I | Q283F | T335F | I372G | I430T |
| Y057V | R116D | P187V | Q233P | Q283W | T335G | I372H | I430V |
| Y057W | R116E | P187W | Q233S | D284C | T335H | I372K | D431P |
| V058A | R116G | P187Y | Q233T | D284I | T335I | I372L | A432C |
| D059A | R116H | D188A | Q234A | D284P | T335K | I372N | A432F |
| D059E | R116I | D188C | Q234D | E285K | T335L | I372P | A432I |
| D059I | R116L | D188F | Q234E | E285P | T335P | I372R | A432K |
| D059L | R116N | D188G | Q234G | E285R | T335V | I372S | A432L |
| D059M | R116P | D188H | Q234H | E285T | T335W | I372T | A432M |
| D059P | R116Q | D188L | Q234N | E285V | T335Y | I372V | A432P |
| D059R | R116S | D188M | Q234P | L286A | L336A | I372W | A432Y |
| D059T | R116V | D188N | Q234S | L286C | L336E | Q373C | L434H |
| D059V | R116W | D188P | Q234T | L286D | L336F | Q373P | L434K |
| D059W | P117D | D188Q | Q234V | L286F | L336G | Q373W | L434P |
| D059Y | P117G | D188R | Q234W | L286H | L336K | L374D | L434Q |
| R060A | P117I | D188S | S235F | L286K | L336N | L374E | L434R |
| R060D | P117K | D188T | S235L | L286M | L336P | E375C | L434W |
| R060F | P117N | D188V | S235M | L286P | L336R | E375F | P437T |
| R060G | P117Q | D188W | S235R | L286T | L336S | E375P | M438Y |
| R060H | P117R | C189A | S235W | L286Y | L336T | E375V | E439N |
| R060I | P117S | C189E | S235Y | V287A | L336V | E375Y | E439R |
| R060L | P117V | C189G | P236C | V287C | R121G | K376I | T440Q |
| R060N | P117W | C189H | W119L | V287D | R121H | K376P | E441R |
| R060P | T118C | C189K | W119N | V287E | R121K | K376W | E442M |
| R060Q | T118D | C189L | W119P | V287G | R121L | G377C | E442N |
| R060S | T118E | C189M | W119R | V287K | R121M | G377I | E442S |
| R060T | T118G | C189N | R121A | V287L | R121P | G377L | P443D |
| T118R | T118P | T118W | R121C | R121F | G378D | G377V | G378E |
| T118Y | W119I | W119A | W119K | R121E | G378F | G378I | |

### Example 5

### Apparent Melting Temperature (Tm) of rHuPH20

In this example, the melting temperature (Tm) of rHuPH20 was determined by measuring the hydrodynamic radius of particles using dynamic light scattering. Particle size increase is presumably due to denaturation and subsequent aggregation of rHuPH20. As temperature increases, proteins will unfold with will lead to aggregate formation.

In brief, rHuPH20 (Lot HuB, 10 mg/mL stock) was diluted to 1 mg/mL in 25 mM Tris-HCl, pH 7.5. Z-average particle size was measured by dynamic light scattering using a Malvern Zeta sizer Nano-ZS as a function of increasing temperature. A total of 3 measurements were made at each temperature in a low volume quartz cuvette (Helma, 3.00 mm) The temperature started at 20° C., with a ramp of 2° C., to a final temperature of 66° C., with a 5 minute equilibration period at each temperature. Light scattering intensity was measured with a 173° backscatter detector equipped with the instrument and the cumulative Z-Average particle data were calculated with the DTS (dispersion technology software) software using a refractive index of 1.45 for the protein samples, and using a refractive index of 1.33 for water as dispersant. The inflection point on the temperature axis at which there is a significant increase in the particle size is considered to be the apparent Tm (melting temperature) where the protein is denatured and begins to aggregate.

The results are shown in Table 9 below, which sets forth the average particle size at various temperatures for rHuPH20. The data in Table 9 are an average of 3 measurements per point, at 2° C. temperature increments, with a 5 minute equilibration point. The results show that the Tm of rHuPH20 is about 44° C.

TABLE 9
Average particle size of rHuPH20

| Temp. (° C.) | rHuPH20 |
|---|---|
| 20 | 9.48 |
| 22 | 9.19 |
| 24 | 9.89 |
| 26 | 9.96 |
| 28 | 9.27 |
| 30 | 8.94 |
| 32 | 8.96 |
| 34 | 9.42 |
| 36 | 9.28 |
| 38 | 8.91 |
| 40 | 9.39 |
| 42 | 9.72 |
| 44 | 11.36 |
| 46 | 13.08 |
| 48 | 21.35 |
| 50 | 22.74 |
| 52 | 28.50 |
| 54 | 32.95 |
| 56 | 37.01 |
| Estimated Tm | 44° C. |

### Example 6

### Temperature Stability Profile of Wildtype PH20 and Variant F204P

1. Wildtype Compared to the F204P-PH20 Variant

Supernatant of expressed wildtype PH20 and PH20 variant F204P generated in Example 2 were collected at 96 hours post-transfection and screened for hyaluronidase activity assay after incubation at various temperatures. Each collected supernatant was incubated for 10 minutes at 4° C., 45° C., 47° C., 49° C., 51° C., 53° C., 55° C. or 57° C., and then cooled on ice. After the incubation, each supernatant was serially diluted (9-fold, 27-fold, 81-fold and 243-fold) and hyaluronidase activity was assessed by the hyaluronidase activity assay as described in Example 3. Duplicate reactions were run for each sample. The reactions were stopped at 5 minutes after addition of TMB, and read immediately using the Molecular Device SPECTRAmax plus at the wavelength of OD 450 nm A standard curve was calculated by using a 4-parameter logistic curve to fit the OD 450 nm data and the estimated activity of the samples was interpolated from the standard curve and dilution factors. The activity of wildtype PH20 or F204P-PH20 at the different temperatures was represented as the percentage of the activity of the particular PH20 in the supernatant incubated at 4° C., which was set at 100%.

The results show that for wildtype PH20 and F204P, incubation at 45° C. and 47° C. resulted in a slight decrease in hyaluronidase activity with about 80% activity remaining after the 10 minute incubation. In contrast, the stability of wildtype PH20 decreased substantially when the supernatant was incubated at temperatures greater than 47° C., whereas the F204P-PH20 variant exhibited greater stability at higher temperatures. For example, wildtype PH20 exhibited about 55%-60% of the hyaluronidase activity after preincubation at 49° C. or 51° C., about 40% of the hyaluronidase activity after preincubation at 53° C., about 20%-25% of the hyaluronidase activity after preincubation at 55° C. and less than 20% of the hyaluronidase activity after preincubation at 57° C. The F204P-PH20 variant in supernatant incubated at 49° C., 51° C., 53° C. or 55° C. exhibited similar activity that was about 60% to 80% of the hyaluronidase activity of the PH20 in the supernatant incubated at 4° C. The hyaluronidase activity of the F204P-PH20 variant in supernatant incubated at 57° C. was about 50% of the hyaluronidase activity of the PH20 in the supernatant incubated at 4° C.

2. F204P Temperature Profile at Higher Temperatures

Since the F204P-PH20 variant exhibited 50% of its hyaluronidase activity at the highest temperature tested of 55° C., the assay was further performed with the F204P-PH20 variant to assess its temperature profile at higher temperatures. The assay as described in part 1 was performed, except that supernatant of expressed F204P-PH20 variant was incubated at 4° C., 55° C., 57° C., 59° C., 61° C., 63° C., 65° C. or 70° C. Similar to the experiment described above, the hyaluronidase activity of the F204P-PH20 variant was about 80% after incubation at 55° C., and was about 60% after incubation at 57° C. The activity of the F204P-PH20 variant in supernatant incubated at temperatures above 57° C. steadily decreased. The hyaluronidase activity of the F204P-PH20 variant in supernatant incubated at 59° C. or 61° C. was about 40%, and the hyaluronidase activity of the F204P-PH20 variant in supernatant incubated at 63° C., 65° C. and 70° C. was about or less than 20%.

### Example 7

### Assay for and Identification of PH20 Uber-Thermophile Variants

Selected PH20 variants from Table 7 that exhibited an activity of 0.4 U/mL or higher were assayed for hyaluronidase activity at 52° C. Specifically, 1,708 different variants expressed in supernatant as described in Example 2 were screened for hyaluronidase activity using the assay described in Example 4 after preincubation of the supernatant at 4° C. or at 52° C. for 10 minutes. PH20 variants that exhibited greater activity after incubation at 52° C. compared to at 4° C. were selected.

1. Primary Screen

Prior to incubating samples with bHA, supernatant containing the tested variant PH20 sample was diluted 1:25 in HEPES assay buffer/transfected supernatant into designated wells of an uncoated 4XHB plate. Two different transfected samples for each variant were used for incubation at each temperature (designated transfection I and transfection II). Thus, each variant was tested in duplicate at both 4° C. and 52° C. The samples were then preincubated at either 4° C. or 52° C. for 10 minutes, and then were cooled before assessing hyaluronidase activity. As a control, wildtype (unmodified) PH20 was used as a control for comparison of activity at each temperature.

The preparation of the bHA coated plates and blocking of the plates prior to addition of the transfected variant supernatants or wildtype PH20 was the same as described in Example 3. A standard curve using rHuPH20 was made as described in Example 3. One hundred microliters (100 μl) of each standard and sample were transferred to pre-designated wells of the bHA-coated and blocked plate and incubated for approximately 1.5 hours at 37° C. Thus, each variant was tested in quadruplicate due to the initial preincubation of two transfected samples expressing the variant (transfection I and transfection II), and then the further use of each sample in duplicate in the bHA assay. After the incubation, the plates were washed and binding to bHA was detected as described above in Example 3. Optical density (OD) was measured at 450 nm within 30 minutes of adding the stop solution.

Table 10 below sets forth the average OD of the duplicate samples for each tested transfected sample at 4° C. and 52° C., and the percent activity remaining at 52° C. compared to 4° C. (% Act. 52° C. vs. 4° C.) for each transfection. The average percent remaining activity of the variants from both transfections also is set forth. For comparison, the Table also depicts the percent activity of wildtype (unmodified) PH20 control at 52° C. compared to 4° C. as tested in the same assay plate. SEQ ID NOS with references to sequences in the Sequence Listing are provided for exemplary variants.

TABLE 10
Hyaluronidase Activity After Incubation at 4° C. and 52° C.

| | Transfection I | | | Transfection II | | | Average | | |
|---|---|---|---|---|---|---|---|---|---|
| | | | % Act. | | | % Act. | % Act. | % Act. | SEQ |
| | | | 52° C. | | | 52° C. | 52° C. | wt 52° C. | ID |
| Variant | 4° C. | 52° C. | vs. 4° C. | 4° C. | 52° C. | vs. 4° C. | vs. 4° C. | vs. 4° C. | NO |
| L001A | 1.33 | 0.47 | 35.60 | 1.22 | 0.34 | 27.88 | 31.74 | 19.42 | |
| L001E | 1.08 | 0.46 | 43.08 | 1.26 | 0.40 | 31.96 | 37.52 | 19.42 | |
| L001F | 1.38 | 0.32 | 23.07 | 1.17 | 0.31 | 26.68 | 24.87 | 17.29 | |
| L001G | 0.17 | 0.05 | 31.29 | 0.10 | 0.06 | 54.90 | 43.09 | 17.29 | |
| L001K | 1.09 | 0.23 | 21.10 | 1.04 | 0.18 | 17.59 | 19.35 | 17.29 | |
| L001N | 0.88 | 0.02 | 1.87 | 0.84 | 0.01 | 1.66 | 1.77 | 17.29 | |
| L001P | 0.35 | 0.12 | 34.80 | 0.36 | 0.11 | 31.34 | 33.07 | 17.29 | |
| L001Q | 1.71 | 0.55 | 32.39 | 2.22 | 0.78 | 35.33 | 33.86 | 30.28 | |
| L001R | 1.79 | 0.63 | 35.22 | 1.63 | 0.42 | 25.72 | 30.47 | 17.29 | |
| L001S | 1.22 | 0.56 | 45.69 | 1.68 | 0.32 | 19.23 | 32.46 | 17.29 | |
| L001T | 2.01 | 0.69 | 34.30 | 2.15 | 0.73 | 33.96 | 34.13 | 11.57 | |
| L001V | 1.51 | 0.47 | 31.39 | 1.22 | 0.43 | 35.59 | 33.49 | 28.17 | |
| L001W | 0.66 | 0.06 | 9.19 | 0.71 | 0.15 | 20.66 | 14.93 | 11.57 | |
| N002Q | 0.91 | 0.23 | 24.75 | 1.02 | 0.26 | 25.36 | 25.05 | 32.12 | |
| N002S | 0.99 | 0.28 | 28.26 | 1.13 | 0.23 | 20.42 | 24.34 | 32.12 | |
| N002V | 0.34 | 0.14 | 41.30 | 0.50 | 0.13 | 26.58 | 33.94 | 32.12 | |
| F003Y | 1.15 | 0.35 | 30.23 | 0.97 | 0.22 | 22.91 | 26.57 | 11.57 | |
| R004T | 0.52 | 0.17 | 31.67 | 0.49 | 0.14 | 28.40 | 30.03 | 32.12 | |
| R004V | 0.35 | 0.18 | 50.57 | 0.38 | 0.14 | 35.89 | 43.23 | 32.12 | |
| A005H | 0.61 | 0.00 | 0.00 | 0.59 | 0.00 | 0.00 | 0.00 | 11.57 | |
| P006A | 1.03 | 0.36 | 35.27 | 0.81 | 0.23 | 28.06 | 31.66 | 11.57 | |
| P006K | 1.44 | 0.50 | 34.59 | 1.32 | 0.40 | 30.38 | 32.48 | 11.57 | |
| P006L | 0.39 | 0.11 | 28.97 | 0.34 | 0.11 | 32.50 | 30.73 | 37.72 | |
| P007M | 0.21 | 0.10 | 46.00 | 0.15 | 0.05 | 31.02 | 38.51 | 31.91 | |
| V008I | 0.75 | 0.28 | 37.87 | 0.78 | 0.28 | 36.22 | 37.05 | 36.38 | |
| V008L | 1.35 | 0.41 | 30.32 | 1.22 | 0.26 | 21.69 | 26.00 | 11.57 | |
| I009Q | 0.37 | 0.14 | 37.35 | 0.22 | 0.11 | 46.99 | 42.17 | 44.87 | |
| I009V | 0.60 | 0.20 | 33.75 | 0.60 | 0.21 | 34.47 | 34.11 | 44.87 | |
| P010D | 0.50 | 0.18 | 35.76 | 0.48 | 0.18 | 36.49 | 36.13 | 44.87 | |
| P010E | 0.65 | 0.23 | 35.45 | 0.67 | 0.12 | 18.58 | 27.01 | 44.87 | |
| P010G | 0.17 | 0.10 | 61.45 | 0.17 | 0.09 | 56.63 | 59.04 | 44.87 | 73 |
| P010N | 0.16 | 0.06 | 38.36 | 0.27 | 0.21 | 76.67 | 57.52 | 32.47 | 74 |
| P010R | 0.28 | 0.08 | 29.84 | 0.21 | 0.09 | 41.69 | 35.76 | 37.72 | |
| N011G | 5.55 | 3.53 | 63.61 | 3.66 | 2.75 | 75.15 | 69.38 | 11.57 | 229 |
| N011K | 0.63 | 0.23 | 36.21 | 1.11 | 0.49 | 43.85 | 40.03 | 2.40 | |
| N011S | 0.61 | 0.07 | 12.23 | 0.87 | 0.33 | 37.93 | 25.08 | 2.40 | |
| V012A | 0.88 | 0.31 | 34.92 | 1.62 | 0.59 | 36.24 | 35.58 | 2.40 | |
| V012E | 0.57 | 0.26 | 44.54 | 0.62 | 0.22 | 35.90 | 40.22 | 36.38 | |
| V012R | 0.57 | 0.00 | 0.00 | 1.36 | 0.00 | 0.00 | 0.00 | 2.40 | |
| V012S | 0.78 | 0.28 | 36.28 | 2.10 | 0.72 | 34.29 | 35.28 | 2.40 | |
| P013H | 0.28 | 0.12 | 42.12 | 0.34 | 0.21 | 61.07 | 51.60 | 27.54 | 76 |
| P013S | 0.48 | 0.20 | 42.44 | 0.46 | 0.16 | 35.71 | 39.08 | 37.91 | |
| P013T | 1.20 | 0.56 | 46.74 | 2.11 | 0.92 | 43.72 | 45.23 | 2.40 | |
| L015A | 0.69 | 0.57 | 82.43 | 0.95 | 0.98 | 103.11 | 92.77 | 30.98 | 132 |
| L015V | 2.42 | 2.50 | 103.33 | 1.98 | 2.13 | 107.86 | 105.60 | 44.87 | 105 |
| A020S | 1.77 | 0.92 | 52.08 | 1.15 | 0.48 | 41.68 | 46.88 | 33.75 | |
| S022H | 0.84 | 0.10 | 11.63 | 0.70 | 0.10 | 13.62 | 12.63 | 14.02 | |
| S022M | 0.28 | 0.10 | 35.33 | 0.29 | 0.10 | 33.85 | 34.59 | 37.72 | |
| S022T | 0.57 | 0.10 | 16.71 | 0.35 | 0.03 | 7.57 | 12.14 | 14.02 | |
| E023D | 0.89 | 0.12 | 13.68 | 0.79 | 0.14 | 17.52 | 15.60 | 14.02 | |
| F024I | 0.53 | 0.07 | 12.71 | 0.47 | 0.08 | 16.81 | 14.76 | 14.02 | |
| F024L | 1.62 | 0.60 | 36.86 | 1.71 | 0.65 | 37.74 | 37.30 | 28.26 | |
| F024M | 0.42 | 0.05 | 12.90 | 0.48 | 0.08 | 15.90 | 14.40 | 14.02 | |
| F024N | 1.08 | 0.25 | 23.34 | 1.00 | 0.26 | 26.43 | 24.89 | 14.02 | |
| F024T | 0.55 | 0.14 | 25.16 | 0.54 | 0.19 | 34.07 | 29.61 | 38.58 | |
| F024V | 0.32 | 0.04 | 12.40 | 0.32 | 0.07 | 22.47 | 17.44 | 17.34 | |
| F024Y | 0.65 | 0.14 | 22.05 | 0.94 | 0.18 | 19.48 | 20.77 | 17.34 | |
| L026A | 1.34 | 0.28 | 20.68 | 1.78 | 0.20 | 11.42 | 16.05 | 17.34 | |
| L026E | 1.21 | 0.20 | 16.08 | 1.27 | 0.14 | 10.85 | 13.47 | 17.34 | |
| L026G | 0.24 | 0.05 | 22.69 | 0.38 | 0.05 | 12.80 | 17.74 | 17.34 | |
| L026H | 0.12 | 0.02 | 12.70 | 0.26 | 0.01 | 5.63 | 9.17 | 17.34 | |
| L026I | 0.10 | 0.03 | 28.95 | 0.20 | 0.06 | 31.06 | 30.00 | 17.34 | |
| L026K | 1.36 | 0.49 | 36.18 | 1.32 | 0.37 | 28.37 | 32.27 | 38.58 | |
| L026M | 1.43 | 0.33 | 23.01 | 1.58 | 0.28 | 17.48 | 20.25 | 38.58 | |
| L026P | 0.89 | 0.52 | 58.94 | 1.00 | 0.59 | 59.03 | 58.98 | 30.98 | 79 |
| L026Q | 3.57 | 1.08 | 30.31 | 3.57 | 1.08 | 30.31 | 30.31 | 32.74 | |
| L026R | 0.64 | 0.59 | 92.63 | 0.74 | 0.66 | 89.23 | 90.93 | 30.98 | 142 |
| L026S | 1.13 | 0.69 | 61.17 | 1.52 | 0.96 | 63.38 | 62.27 | 30.98 | 334 |
| L026T | 0.98 | 0.31 | 31.26 | 1.71 | 0.79 | 45.95 | 38.61 | 32.74 | |
| L026V | 0.35 | 0.15 | 42.45 | 0.55 | 0.35 | 63.16 | 52.80 | 32.74 | 82 |
| L026W | 0.29 | 0.12 | 39.69 | 0.75 | 0.52 | 68.35 | 54.02 | 32.74 | 83 |
| L026Y | 0.82 | 0.27 | 32.66 | 1.39 | 1.06 | 76.14 | 54.40 | 32.74 | 84 |
| G027A | 3.19 | 1.43 | 44.85 | 2.35 | 1.00 | 42.33 | 43.59 | 34.04 | |
| G027E | 0.15 | 0.27 | 173.14 | 0.24 | 0.32 | 133.75 | 153.44 | 30.98 | 290 |
| G027F | 0.67 | 0.27 | 40.07 | 0.75 | 0.26 | 35.14 | 37.61 | 36.38 | |
| G027F | 0.53 | 0.12 | 22.34 | 0.53 | 0.08 | 14.59 | 18.47 | 38.58 | |
| V206I | 0.19 | 0.12 | 62.63 | 0.17 | 0.10 | 58.86 | 60.74 | 30.73 | 269 |
| G027I | 0.95 | 0.37 | 38.73 | 0.70 | 0.21 | 29.74 | 34.24 | 21.53 | |
| G027K | 0.33 | 0.14 | 42.12 | 0.26 | 0.12 | 45.49 | 43.81 | 32.94 | |
| G027L | 0.33 | 0.11 | 32.07 | 0.24 | 0.07 | 30.25 | 31.16 | 34.04 | |
| G027Q | 2.01 | 0.58 | 28.83 | 1.67 | 0.36 | 21.45 | 25.14 | 34.04 | |
| G027R | 2.26 | 0.63 | 27.69 | 3.95 | 1.70 | 42.97 | 35.33 | 32.94 | |
| G027S | 0.65 | 0.27 | 42.07 | 0.51 | 0.22 | 43.24 | 42.65 | 34.04 | |
| G027T | 0.85 | 0.40 | 46.61 | 0.67 | 0.23 | 34.08 | 40.34 | 21.53 | |
| G027W | 0.91 | 0.29 | 31.38 | 0.93 | 0.37 | 39.78 | 35.58 | 21.54 | |
| K028A | 0.80 | 0.44 | 55.10 | 0.67 | 0.29 | 43.24 | 49.17 | 21.53 | |
| K028D | 0.82 | 0.33 | 41.04 | 0.81 | 0.29 | 35.87 | 38.46 | 36.38 | |
| K028E | 0.38 | 0.16 | 42.67 | 0.32 | 0.13 | 41.45 | 42.06 | 32.94 | |
| K028F | 0.46 | 0.09 | 20.37 | 0.41 | 0.11 | 27.11 | 23.74 | 21.54 | |
| K028I | 0.18 | 0.06 | 32.58 | 0.14 | 0.08 | 57.65 | 45.12 | 21.54 | |
| K028L | 0.51 | 0.36 | 71.32 | 0.44 | 0.15 | 32.85 | 52.08 | 21.53 | 87 |
| K028M | 0.47 | 0.23 | 48.87 | 0.47 | 0.14 | 29.74 | 39.31 | 21.53 | |
| K028N | 0.50 | 0.15 | 29.26 | 0.37 | 0.11 | 30.81 | 30.03 | 21.54 | |
| K028R | 0.63 | 0.21 | 32.70 | 0.56 | 0.14 | 25.60 | 29.15 | 32.94 | |
| K028S | 0.90 | 0.30 | 33.54 | 0.99 | 0.36 | 35.85 | 34.69 | 28.26 | |
| K028T | 1.14 | 0.26 | 22.59 | 1.07 | 0.22 | 20.48 | 21.53 | 33.16 | |
| K028V | 0.11 | 0.03 | 31.43 | 0.09 | 0.05 | 52.60 | 42.01 | 33.16 | |
| K028W | 0.43 | 0.19 | 44.01 | 0.45 | 0.12 | 25.83 | 34.92 | 21.53 | |
| F029A | 0.95 | 0.29 | 30.63 | 0.81 | 0.25 | 30.24 | 30.43 | 33.16 | |
| F029E | 1.54 | 0.89 | 57.70 | 1.44 | 0.62 | 42.72 | 50.21 | 21.53 | 88 |
| S261A | 0.62 | 0.67 | 107.59 | 0.98 | 0.43 | 43.95 | 75.77 | 26.81 | 196 |
| F029I | 4.84 | 1.67 | 34.54 | 5.64 | 1.66 | 29.40 | 31.97 | 33.16 | |
| F029K | 0.95 | 0.40 | 41.87 | 2.24 | 0.71 | 31.50 | 36.69 | 36.21 | |
| F029L | 0.43 | 0.24 | 55.85 | 0.42 | 0.23 | 55.11 | 55.48 | 36.21 | 90 |
| F029M | 0.92 | 0.35 | 38.44 | 1.16 | 0.47 | 40.77 | 39.60 | 36.21 | |
| F029P | 0.64 | 0.28 | 43.75 | 0.74 | 0.35 | 47.53 | 45.64 | 36.21 | |
| F029R | 0.76 | 0.23 | 30.21 | 0.71 | 0.21 | 29.46 | 29.83 | 32.94 | |
| F029S | 0.28 | 0.20 | 73.33 | 0.26 | 0.24 | 91.60 | 82.47 | 36.21 | 217 |
| F029T | 0.67 | 0.22 | 32.19 | 0.63 | 0.19 | 29.31 | 30.75 | 32.94 | |
| F029V | 1.99 | 0.54 | 27.09 | 2.17 | 0.63 | 29.01 | 28.05 | 36.21 | |
| F029W | 0.28 | 0.15 | 52.38 | 0.42 | 0.21 | 51.02 | 51.70 | 36.21 | 92 |
| D030A | 0.34 | 0.23 | 65.74 | 0.30 | 0.20 | 66.50 | 66.12 | 36.75 | 329 |
| D030F | 1.94 | 0.57 | 29.25 | 2.04 | 0.57 | 28.14 | 28.70 | 36.75 | |
| D030G | 3.30 | 1.15 | 34.99 | 3.94 | 1.41 | 35.73 | 35.36 | 36.75 | |
| D030H | 3.04 | 0.94 | 30.85 | 3.47 | 0.97 | 27.95 | 29.40 | 36.75 | |
| D030K | 0.61 | 0.17 | 27.66 | 0.56 | 0.13 | 22.31 | 24.99 | 32.94 | |
| D030L | 1.93 | 0.47 | 24.59 | 2.06 | 0.49 | 23.57 | 24.08 | 36.75 | |
| D030M | 2.55 | 0.78 | 30.40 | 3.19 | 0.79 | 24.69 | 27.54 | 36.75 | |
| D284A | 0.18 | 0.35 | 191.48 | 0.19 | 0.32 | 170.35 | 180.92 | 37.53 | 131 |
| D030Q | 1.74 | 0.36 | 20.78 | 1.48 | 0.32 | 21.53 | 21.15 | 38.58 | |
| D030R | 7.13 | 3.09 | 43.40 | 8.55 | 6.47 | 75.72 | 59.56 | 45.75 | 94 |
| D030S | 4.61 | 1.41 | 30.64 | 7.97 | 3.90 | 48.92 | 39.78 | 45.75 | |
| D030T | 3.23 | 1.04 | 32.29 | 5.37 | 2.89 | 53.90 | 43.09 | 45.75 | |
| D030V | 0.31 | 0.14 | 46.03 | 0.28 | 0.14 | 50.62 | 48.33 | 37.91 | |
| D030W | 1.04 | 0.40 | 38.54 | 1.68 | 0.89 | 52.72 | 45.63 | 45.75 | |
| E031A | 0.22 | 0.07 | 33.56 | 0.51 | 0.32 | 62.35 | 47.96 | 45.75 | |
| E031C | 4.81 | 1.83 | 38.00 | 9.55 | 6.68 | 69.94 | 53.97 | 45.75 | 95 |
| E031G | 3.24 | 1.00 | 30.78 | 4.44 | 8.22 | 185.23 | 108.00 | 45.75 | 103 |
| E031H | 0.30 | 0.00 | 0.00 | 0.27 | 0.13 | 47.67 | 23.84 | 30.28 | |
| E031I | 1.08 | 0.45 | 41.96 | 1.25 | 0.48 | 38.15 | 40.06 | 30.73 | |
| E031K | 3.39 | 1.93 | 56.82 | 1.91 | 0.70 | 36.70 | 46.76 | 41.87 | |
| E031L | 0.22 | 0.20 | 89.75 | 0.21 | 0.12 | 55.13 | 72.44 | 41.87 | 380 |
| E031P | 1.04 | 0.53 | 51.08 | 1.10 | 0.30 | 27.17 | 39.12 | 41.87 | |
| E031R | 4.09 | 1.59 | 39.01 | 3.94 | 1.18 | 29.98 | 34.50 | 41.87 | |
| E031S | 1.67 | 0.30 | 17.72 | 1.62 | 0.25 | 15.61 | 16.66 | 38.58 | |
| E031T | 3.66 | 0.76 | 20.85 | 2.19 | 0.54 | 24.76 | 22.81 | 40.82 | |
| E031V | 8.50 | 1.09 | 12.82 | 6.60 | 0.89 | 13.49 | 13.16 | 38.58 | |
| E031W | 1.36 | 0.63 | 46.13 | 1.33 | 0.29 | 21.50 | 33.82 | 41.87 | |
| E031Y | 1.45 | 0.58 | 40.15 | 1.12 | 0.32 | 28.37 | 34.26 | 41.87 | |
| P032A | 6.34 | 2.32 | 36.55 | 6.03 | 1.01 | 16.71 | 26.63 | 41.87 | |
| P032G | 1.95 | 0.90 | 46.27 | 1.85 | 0.76 | 40.91 | 43.59 | 35.82 | |
| P032K | 4.19 | 1.50 | 35.68 | 2.95 | 1.07 | 36.15 | 35.92 | 28.17 | |
| P032L | 0.83 | 0.26 | 31.55 | 0.84 | 0.25 | 29.50 | 30.52 | 28.15 | |
| P032M | 1.40 | 0.49 | 35.22 | 1.46 | 0.43 | 29.68 | 32.45 | 35.82 | |
| P032N | 0.99 | 0.36 | 36.12 | 1.20 | 0.35 | 29.21 | 32.67 | 28.15 | |
| P032Q | 0.65 | 0.45 | 68.76 | 0.64 | 0.50 | 78.03 | 73.40 | 35.82 | 200 |
| P032R | 1.28 | 0.54 | 41.78 | 1.07 | 0.46 | 43.00 | 42.39 | 35.82 | |
| P032S | 1.03 | 0.50 | 48.11 | 0.88 | 0.48 | 55.12 | 51.61 | 35.82 | 99 |
| P032T | 2.01 | 0.73 | 36.52 | 1.90 | 0.63 | 33.17 | 34.84 | 35.82 | |
| P032V | 0.23 | 0.11 | 46.10 | 0.15 | 0.08 | 55.78 | 50.94 | 34.09 | 100 |
| P032W | 0.55 | 0.35 | 63.21 | 0.54 | 0.33 | 61.07 | 62.14 | 29.51 | 337 |
| P032Y | 2.25 | 0.66 | 29.35 | 1.52 | 0.31 | 20.64 | 24.99 | 32.61 | |
| L033G | 0.21 | 0.27 | 126.89 | 0.20 | 0.29 | 144.75 | 135.82 | 29.51 | 149 |
| L033M | 0.22 | 0.35 | 164.50 | 0.25 | 0.31 | 121.74 | 143.12 | 29.51 | 150 |
| L033Q | 1.04 | 0.35 | 33.49 | 1.09 | 0.30 | 27.69 | 30.59 | 28.15 | |
| L033R | 0.24 | 0.33 | 139.71 | 0.18 | 0.63 | 342.47 | 241.09 | 29.51 | 151 |
| L033W | 0.58 | 0.39 | 67.85 | 0.47 | 0.51 | 108.62 | 88.23 | 29.51 | 154 |
| D034E | 0.44 | 0.43 | 99.31 | 0.49 | 0.34 | 69.12 | 84.22 | 29.51 | 182 |
| D034H | 0.19 | 0.40 | 205.94 | 0.31 | 0.57 | 183.44 | 194.69 | 29.51 | 155 |
| D034Q | 0.31 | 0.16 | 52.44 | 0.33 | 0.14 | 43.55 | 47.99 | 30.73 | |
| Q143K | 0.59 | 0.43 | 73.53 | 0.62 | 0.42 | 66.69 | 70.11 | 36.07 | 221 |
| M035F | 1.81 | 0.40 | 22.18 | 1.70 | 0.38 | 22.47 | 22.32 | 23.34 | |
| M035L | 0.70 | 0.28 | 39.58 | 0.83 | 0.26 | 31.93 | 35.75 | 23.34 | |
| M035Q | 1.22 | 0.30 | 24.26 | 1.03 | 0.26 | 24.94 | 24.60 | 23.34 | |
| M035T | 1.34 | 0.31 | 22.96 | 1.23 | 0.28 | 23.10 | 23.03 | 23.34 | |
| M035V | 0.78 | 0.29 | 36.72 | 0.81 | 0.26 | 31.87 | 34.30 | 23.34 | |
| M035Y | 1.75 | 0.46 | 26.18 | 2.13 | 0.49 | 23.10 | 24.64 | 23.34 | |
| S036G | 0.66 | 0.31 | 46.78 | 0.63 | 0.36 | 57.09 | 51.93 | 36.50 | 109 |
| S036H | 0.53 | 0.24 | 45.20 | 0.65 | 0.29 | 45.08 | 45.14 | 36.50 | |
| S036K | 0.51 | 0.26 | 51.66 | 0.80 | 0.32 | 40.02 | 45.84 | 36.50 | |
| S036R | 0.84 | 0.29 | 34.09 | 1.14 | 0.34 | 29.55 | 31.82 | 36.50 | |
| S036T | 0.76 | 0.32 | 42.31 | 0.97 | 0.35 | 36.51 | 39.41 | 36.50 | |
| L037F | 0.74 | 0.39 | 51.88 | 0.97 | 0.39 | 40.50 | 46.19 | 36.50 | |
| L037I | 0.26 | 0.13 | 51.54 | 0.23 | 0.13 | 56.26 | 53.90 | 30.73 | 110 |
| S315T | 1.63 | 2.36 | 144.57 | 1.95 | 1.13 | 57.57 | 101.07 | 21.73 | 115 |
| L037M | 0.42 | 0.19 | 46.48 | 0.38 | 0.15 | 40.58 | 43.53 | 30.73 | |
| F038Y | 0.63 | 0.29 | 46.49 | 0.55 | 0.64 | 117.23 | 81.86 | 26.61 | 188 |
| S039A | 1.40 | 0.66 | 46.78 | 3.16 | 1.27 | 40.32 | 43.55 | 26.61 | |
| S039N | 2.66 | 0.97 | 36.24 | 1.98 | 1.01 | 50.86 | 43.55 | 26.61 | |
| S039Q | 1.82 | 0.74 | 40.51 | 0.82 | 0.68 | 82.47 | 61.49 | 26.61 | 368 |
| S039R | 0.82 | 0.54 | 65.81 | 0.46 | 0.56 | 122.67 | 94.24 | 26.61 | 128 |
| S039T | 0.78 | 0.56 | 72.32 | 1.70 | 0.91 | 53.51 | 62.91 | 26.61 | 300 |
| F040L | 3.93 | 0.70 | 17.86 | 3.22 | 0.80 | 24.75 | 21.31 | 31.60 | |
| F040W | 2.17 | 1.11 | 51.21 | 1.34 | 0.36 | 26.79 | 39.00 | 27.87 | |
| I041A | 0.92 | 0.25 | 27.11 | 0.88 | 0.22 | 24.53 | 25.82 | 24.78 | |
| I041D | 0.14 | 0.10 | 72.43 | 0.18 | 0.10 | 53.30 | 62.86 | 24.78 | 166 |
| I041D | 0.18 | 0.13 | 72.85 | 0.17 | 0.11 | 61.38 | 67.12 | 28.63 | 166 |
| I041G | 0.16 | 0.00 | 0.00 | 0.23 | 0.00 | 0.00 | 0.00 | 20.11 | |
| I041N | 0.60 | 0.22 | 37.23 | 0.82 | 0.26 | 31.15 | 34.19 | 24.78 | |
| I041T | 0.28 | 0.21 | 76.31 | 0.51 | 0.24 | 47.52 | 61.92 | 24.78 | 167 |
| I041V | 3.30 | 1.11 | 33.66 | 3.60 | 1.15 | 31.99 | 32.83 | 24.78 | |
| I041W | 0.34 | 0.20 | 57.10 | 0.83 | 0.78 | 94.22 | 75.66 | 16.82 | 126 |
| S043N | 0.28 | 0.17 | 60.92 | 0.38 | 0.13 | 32.68 | 46.80 | 28.63 | |
| P044E | 0.27 | 0.00 | 0.00 | 0.28 | 0.00 | 0.00 | 0.00 | 20.11 | |
| R045K | 1.02 | 0.43 | 41.74 | 2.95 | 1.32 | 44.67 | 43.20 | 16.82 | |
| I046A | 1.53 | 0.60 | 39.52 | 3.77 | 1.56 | 41.52 | 40.52 | 16.82 | |
| I046C | 0.42 | 0.18 | 43.10 | 0.81 | 0.28 | 34.99 | 39.04 | 27.59 | |
| I046F | 0.44 | 0.22 | 49.89 | 1.08 | 0.33 | 30.35 | 40.12 | 27.59 | |
| I046H | 0.31 | 0.17 | 54.57 | 0.38 | 0.18 | 47.42 | 51.00 | 27.59 | 120 |
| I046L | 0.49 | 0.23 | 45.55 | 0.90 | 0.35 | 39.25 | 42.40 | 27.59 | |
| I046M | 0.51 | 0.23 | 46.29 | 0.38 | 0.16 | 43.06 | 44.68 | 27.59 | |
| I046N | 0.43 | 0.15 | 34.45 | 0.44 | 0.17 | 39.26 | 36.85 | 35.61 | |
| I046R | 2.80 | 0.72 | 25.78 | 2.78 | 0.62 | 22.36 | 24.07 | 35.61 | |
| I046S | 0.20 | 0.09 | 46.06 | 0.22 | 0.08 | 34.88 | 40.47 | 35.61 | |
| I046T | 0.63 | 0.23 | 35.89 | 0.77 | 0.23 | 29.50 | 32.70 | 35.61 | |
| I046V | 0.84 | 0.04 | 4.23 | 0.95 | 0.03 | 3.49 | 3.86 | 20.11 | |
| I046Y | 1.10 | 0.31 | 28.47 | 1.00 | 0.27 | 26.53 | 27.50 | 35.61 | |
| N047A | 0.40 | 0.16 | 40.05 | 0.41 | 0.12 | 28.33 | 34.19 | 35.61 | |
| N047G | 0.28 | 0.12 | 40.99 | 0.27 | 0.16 | 59.29 | 50.14 | 35.61 | 121 |
| N047H | 0.93 | 0.25 | 27.29 | 0.78 | 0.14 | 17.45 | 22.37 | 16.92 | |
| N047M | 0.77 | 0.24 | 31.37 | 0.62 | 0.16 | 24.98 | 28.18 | 32.61 | |
| N047Q | 0.32 | 0.00 | 0.00 | 0.41 | 0.03 | 7.49 | 3.75 | 16.92 | |
| N047R | 0.40 | 0.27 | 66.54 | 0.52 | 0.23 | 43.94 | 55.24 | 28.04 | 122 |
| N047S | 0.39 | 0.06 | 14.32 | 0.43 | 0.00 | 0.00 | 7.16 | 16.92 | |
| N047T | 0.41 | 0.06 | 15.63 | 0.50 | 0.00 | 0.70 | 8.16 | 16.92 | |
| N047W | 0.58 | 0.17 | 28.93 | 0.46 | 0.12 | 26.89 | 27.91 | 32.61 | |
| N047Y | 0.36 | 0.02 | 5.33 | 0.44 | 0.00 | 0.79 | 3.06 | 16.92 | |
| S215T | 0.76 | 0.49 | 64.96 | 1.08 | 0.48 | 44.12 | 54.54 | 19.93 | 246 |
| A048H | 3.68 | 0.98 | 26.71 | 2.87 | 0.62 | 21.73 | 24.22 | 32.61 | |
| A048I | 2.57 | 0.26 | 9.91 | 2.09 | 0.43 | 20.81 | 15.36 | 16.92 | |
| A048K | 2.02 | 1.00 | 49.21 | 2.32 | 0.87 | 37.69 | 43.45 | 28.04 | |
| A048M | 2.49 | 0.76 | 30.61 | 2.33 | 0.66 | 28.23 | 29.42 | 22.27 | |
| A048N | 9.51 | 2.68 | 28.16 | 6.95 | 1.48 | 21.29 | 24.73 | 32.61 | |
| A048Q | 1.79 | 0.80 | 44.80 | 2.02 | 0.74 | 36.67 | 40.73 | 28.04 | |
| A048R | 3.54 | 1.06 | 29.85 | 3.42 | 0.88 | 25.78 | 27.82 | 22.27 | |
| A048S | 0.95 | 0.36 | 38.28 | 0.86 | 0.30 | 35.49 | 36.89 | 22.27 | |
| A048V | 1.11 | 0.48 | 43.53 | 1.25 | 0.50 | 39.80 | 41.67 | 28.04 | |
| A048Y | 0.56 | 0.34 | 60.30 | 0.77 | 0.31 | 40.39 | 50.35 | 28.04 | 124 |
| T049I | 0.17 | 0.09 | 51.62 | 0.16 | 0.09 | 57.45 | 54.54 | 29.04 | 125 |
| T049K | 1.18 | 0.31 | 26.23 | 1.28 | 0.31 | 24.63 | 25.43 | 29.04 | |
| T049R | 1.84 | 0.45 | 24.61 | 1.80 | 0.40 | 22.21 | 23.41 | 29.04 | |
| T049S | 1.56 | 0.67 | 42.94 | 1.18 | 0.55 | 46.39 | 44.67 | 29.23 | |
| T049V | 0.23 | 0.00 | 0.00 | 0.32 | 0.00 | 0.00 | 0.00 | 20.11 | |
| G050A | 0.52 | 0.19 | 37.20 | 0.50 | 0.20 | 40.40 | 38.80 | 29.04 | |
| G050C | 0.13 | 0.09 | 70.12 | 0.12 | 0.10 | 89.57 | 79.84 | 27.69 | 195 |
| G050D | 0.50 | 0.31 | 61.80 | 0.25 | 0.15 | 61.54 | 61.67 | 27.69 | 364 |
| G050E | 0.79 | 0.38 | 47.38 | 0.58 | 0.25 | 42.65 | 45.01 | 29.04 | |
| G050H | 1.38 | 0.58 | 41.71 | 4.49 | 0.58 | 12.85 | 27.28 | 25.07 | |
| G050L | 0.36 | 0.19 | 53.28 | 0.50 | 0.23 | 45.73 | 49.50 | 27.69 | |
| G050Q | 0.69 | 0.20 | 28.93 | 1.82 | 0.67 | 36.94 | 32.94 | 25.07 | |
| G050R | 0.91 | 0.25 | 27.26 | 2.07 | 0.25 | 11.98 | 19.62 | 25.07 | |
| G050S | 0.15 | 0.01 | 3.41 | 0.22 | 0.04 | 16.67 | 10.04 | 25.07 | |
| G050Y | 0.25 | 0.07 | 30.14 | 0.77 | 0.20 | 25.70 | 27.92 | 25.07 | |
| Q051A | 0.20 | 0.01 | 6.47 | 0.19 | 0.00 | 0.00 | 3.23 | 25.07 | |
| Q051N | 1.09 | 0.06 | 5.51 | 1.02 | 0.00 | 0.00 | 2.76 | 27.17 | |
| G052N | 1.91 | 1.02 | 53.50 | 2.05 | 0.82 | 39.74 | 46.62 | 27.17 | |
| G052Q | 0.83 | 0.33 | 39.65 | 0.64 | 0.20 | 30.33 | 34.99 | 32.61 | |
| G052S | 0.81 | 0.30 | 36.62 | 1.18 | 0.38 | 32.30 | 34.46 | 27.17 | |
| G052T | 0.85 | 0.43 | 50.09 | 1.01 | 0.34 | 33.98 | 42.03 | 27.17 | |
| T054A | 0.50 | 0.06 | 11.64 | 0.61 | 0.02 | 3.96 | 7.80 | 27.17 | |
| T054N | 0.80 | 0.42 | 52.90 | 0.99 | 0.39 | 39.05 | 45.98 | 27.17 | |
| T054Q | 3.82 | 1.60 | 42.01 | 4.61 | 1.50 | 32.54 | 37.27 | 27.17 | |
| T054S | 3.41 | 1.38 | 40.34 | 2.43 | 0.43 | 17.75 | 29.04 | 27.17 | |
| T054V | 1.00 | 0.00 | 0.00 | 0.43 | 0.00 | 0.00 | 0.00 | 11.45 | |
| V058C | 1.17 | 0.12 | 10.58 | 0.43 | 0.00 | 0.00 | 5.29 | 11.45 | |
| V058G | 0.99 | 0.01 | 1.41 | 0.44 | 0.00 | 0.00 | 0.70 | 11.45 | |
| V058H | 2.25 | 0.54 | 24.13 | 1.10 | 0.24 | 21.91 | 23.02 | 11.45 | |
| V058I | 2.45 | 0.29 | 11.65 | 1.68 | 0.20 | 11.75 | 11.70 | 11.45 | |
| S235T | 1.32 | 0.86 | 65.16 | 1.87 | 1.41 | 75.41 | 70.28 | 31.65 | 218 |
| V058L | 2.39 | 0.81 | 33.74 | 1.41 | 0.44 | 31.51 | 32.63 | 11.45 | |
| V058N | 1.28 | 0.46 | 35.75 | 1.23 | 0.40 | 32.51 | 34.13 | 36.38 | |
| V058P | 1.33 | 0.64 | 47.73 | 1.16 | 0.41 | 35.75 | 41.74 | 26.81 | |
| V058Q | 4.89 | 1.88 | 38.39 | 3.99 | 1.41 | 35.39 | 36.89 | 26.81 | |
| F204P | 2.72 | 4.19 | 154.20 | 1.84 | 1.13 | 61.20 | 107.70 | 31.65 | 104 |
| V058S | 1.77 | 0.87 | 49.01 | 1.56 | 0.52 | 33.11 | 41.06 | 26.81 | |
| V058Y | 1.03 | 0.40 | 39.16 | 1.09 | 0.50 | 45.81 | 42.49 | 26.81 | |
| D059N | 2.29 | 0.58 | 25.47 | 2.33 | 0.43 | 18.57 | 22.02 | 26.81 | |
| T147I | 0.63 | 0.52 | 82.89 | 0.56 | 0.48 | 85.17 | 84.03 | 34.46 | 183 |
| Y063H | 1.32 | 0.33 | 25.21 | 1.55 | 0.16 | 10.28 | 17.74 | 0.00 | |
| Y063I | 1.49 | 0.00 | 0.00 | 1.75 | 0.03 | 1.94 | 0.97 | 0.00 | |
| Y063K | 5.75 | 0.23 | 3.95 | 5.31 | 0.01 | 0.14 | 2.04 | 0.00 | |
| Y063L | 1.61 | 0.00 | 0.00 | 2.27 | 0.00 | 0.00 | 0.00 | 17.25 | |
| Y063M | 0.80 | 0.00 | 0.00 | 0.01 | 0.00 | 0.00 | 0.00 | 17.25 | |
| Y063N | 0.57 | 0.18 | 31.27 | 0.72 | 0.21 | 29.59 | 30.43 | 36.38 | |
| Y063R | 2.64 | 0.34 | 12.97 | 3.51 | 0.12 | 3.36 | 8.17 | 17.25 | |
| Y063S | 0.37 | 0.00 | 0.00 | 0.48 | 0.00 | 0.00 | 0.00 | 17.25 | |
| Y063T | 0.64 | 0.00 | 0.00 | 1.17 | 0.00 | 0.00 | 0.00 | 17.25 | |
| Y063W | 1.54 | 0.00 | 0.00 | 1.60 | 0.21 | 12.82 | 6.41 | 17.25 | |
| Y066H | 1.00 | 0.18 | 17.97 | 1.41 | 0.03 | 2.06 | 10.02 | 17.25 | |
| Y066R | 1.72 | 0.19 | 10.98 | 1.24 | 0.03 | 2.21 | 6.59 | 12.63 | |
| I067F | 0.35 | 0.23 | 67.63 | 0.34 | 0.19 | 56.79 | 62.21 | 32.47 | 359 |
| I067V | 2.56 | 0.81 | 31.62 | 2.22 | 0.42 | 19.03 | 25.33 | 12.63 | |
| I067Y | 1.72 | 0.45 | 26.07 | 0.84 | 0.18 | 21.86 | 23.97 | 35.76 | |
| D068E | 1.31 | 0.35 | 26.68 | 1.18 | 0.16 | 13.87 | 20.27 | 12.63 | |
| D068H | 5.10 | 1.31 | 25.69 | 4.62 | 0.89 | 19.34 | 22.52 | 12.63 | |
| D068K | 3.45 | 0.61 | 17.61 | 3.05 | 0.33 | 10.85 | 14.23 | 12.63 | |
| D068Q | 3.87 | 0.51 | 13.20 | 3.52 | 0.57 | 16.24 | 14.72 | 12.63 | |
| D068R | 4.42 | 0.36 | 8.12 | 3.44 | 0.36 | 10.57 | 9.34 | 12.63 | |
| D068S | 0.91 | 0.02 | 2.09 | 0.96 | 0.03 | 3.38 | 2.73 | 22.90 | |
| S069A | 0.34 | 0.25 | 72.79 | 0.31 | 0.27 | 87.16 | 79.98 | 32.47 | 360 |
| S069E | 0.25 | 0.13 | 53.36 | 0.29 | 0.04 | 13.49 | 33.42 | 22.90 | |
| S069G | 0.31 | 0.16 | 49.92 | 0.44 | 0.14 | 31.69 | 40.80 | 22.90 | |
| S069M | 0.78 | 0.43 | 54.28 | 1.01 | 0.36 | 35.79 | 45.03 | 22.90 | |
| S069P | 1.70 | 0.86 | 50.58 | 2.00 | 0.92 | 46.29 | 48.43 | 22.90 | |
| S069R | 3.01 | 1.08 | 35.84 | 3.78 | 0.89 | 23.52 | 29.68 | 22.90 | |
| S069T | 1.34 | 0.47 | 34.85 | 1.28 | 0.52 | 40.14 | 37.50 | 29.23 | |
| S069W | 0.72 | 0.19 | 26.16 | 0.55 | 0.15 | 27.61 | 26.89 | 28.17 | |
| S069Y | 0.42 | 0.30 | 70.83 | 0.46 | 0.27 | 58.14 | 64.49 | 30.17 | 278 |
| I070A | 0.56 | 0.30 | 53.30 | 0.54 | 0.23 | 42.25 | 47.78 | 30.17 | |
| I070C | 0.96 | 0.50 | 52.58 | 1.12 | 0.30 | 27.03 | 39.81 | 30.17 | |
| I070F | 1.78 | 0.66 | 36.90 | 1.43 | 0.59 | 41.42 | 39.16 | 30.17 | |
| I070H | 2.66 | 0.91 | 33.99 | 1.81 | 0.56 | 30.70 | 32.35 | 30.17 | |
| I070K | 0.66 | 0.23 | 34.62 | 0.42 | 0.26 | 61.16 | 47.89 | 30.17 | |
| I070L | 0.81 | 0.13 | 16.03 | 0.72 | 0.16 | 21.77 | 18.90 | 30.17 | |
| I070N | 0.88 | 0.28 | 32.02 | 0.97 | 0.39 | 39.63 | 35.83 | 30.17 | |
| I070P | 0.29 | 0.00 | 0.00 | 0.21 | 0.00 | 0.00 | 0.00 | 5.42 | |
| I070R | 2.38 | 0.52 | 21.99 | 2.18 | 0.49 | 22.44 | 22.22 | 35.76 | |
| I070S | 1.54 | 0.34 | 22.27 | 1.58 | 0.30 | 19.23 | 20.75 | 35.76 | |
| I070T | 0.28 | 0.00 | 0.00 | 0.27 | 0.00 | 0.00 | 0.00 | 5.42 | |
| I070V | 1.21 | 0.29 | 23.82 | 1.07 | 0.30 | 27.65 | 25.73 | 35.76 | |
| T071A | 0.24 | 0.00 | 0.00 | 0.46 | 0.00 | 0.00 | 0.00 | 5.42 | |
| T071G | 0.50 | 0.00 | 0.00 | 0.67 | 0.00 | 0.00 | 0.00 | 20.11 | |
| T071N | 1.03 | 0.22 | 21.55 | 0.90 | 0.18 | 19.92 | 20.74 | 35.76 | |
| T071R | 1.29 | 0.18 | 14.10 | 1.25 | 0.01 | 0.68 | 7.39 | 5.42 | |
| T071S | 1.19 | 0.35 | 29.52 | 1.05 | 0.20 | 19.52 | 24.52 | 5.42 | |
| G072A | 0.89 | 0.17 | 19.10 | 0.87 | 0.15 | 17.17 | 18.13 | 28.16 | |
| G072D | 3.07 | 2.61 | 84.94 | 2.54 | 0.74 | 29.27 | 57.11 | 32.61 | 133 |
| G072H | 0.75 | 0.21 | 27.64 | 0.72 | 0.20 | 28.37 | 28.01 | 29.23 | |
| G072K | 0.62 | 0.13 | 20.99 | 0.64 | 0.13 | 19.94 | 20.46 | 28.16 | |
| G072Q | 0.59 | 0.00 | 0.00 | 0.84 | 0.00 | 0.00 | 0.00 | 20.11 | |
| G072R | 1.26 | 0.22 | 17.67 | 1.71 | 0.24 | 14.17 | 15.92 | 28.16 | |
| G072S | 0.67 | 0.16 | 24.02 | 0.98 | 0.18 | 18.83 | 21.43 | 28.16 | |
| V073A | 0.75 | 0.26 | 35.00 | 0.63 | 0.24 | 38.50 | 36.75 | 5.42 | |
| V073D | 1.74 | 0.81 | 46.42 | 1.92 | 0.65 | 33.59 | 40.01 | 5.42 | |
| V073G | 0.49 | 0.13 | 26.62 | 0.61 | 0.05 | 8.72 | 17.67 | 24.32 | |
| V073H | 0.99 | 0.27 | 27.22 | 0.62 | 0.34 | 53.90 | 40.56 | 24.32 | |
| V073M | 1.69 | 0.59 | 35.03 | 1.87 | 0.54 | 28.82 | 31.92 | 24.32 | |
| V073Q | 0.86 | 0.44 | 51.57 | 1.01 | 0.40 | 39.11 | 45.34 | 24.32 | |
| V073S | 1.85 | 0.75 | 40.53 | 1.84 | 0.52 | 28.05 | 34.29 | 24.32 | |
| V073T | 0.44 | 0.26 | 58.68 | 0.55 | 0.26 | 47.04 | 52.86 | 24.32 | 134 |
| V073W | 1.55 | 0.69 | 44.69 | 1.92 | 0.75 | 38.98 | 41.84 | 24.32 | |
| T074C | 1.42 | 0.78 | 55.12 | 1.84 | 0.69 | 37.65 | 46.39 | 24.32 | |
| T074F | 1.23 | 0.38 | 31.06 | 1.55 | 0.65 | 41.58 | 36.32 | 28.96 | |
| T074H | 0.94 | 0.11 | 12.21 | 0.87 | 0.40 | 45.79 | 29.00 | 28.96 | |
| T074K | 1.54 | 0.21 | 13.77 | 1.94 | 0.30 | 15.53 | 14.65 | 28.96 | |
| T074M | 1.98 | 0.49 | 24.84 | 2.07 | 0.50 | 24.01 | 24.43 | 29.23 | |
| T074N | 1.78 | 0.51 | 28.40 | 2.06 | 0.64 | 30.85 | 29.62 | 28.96 | |
| T074P | 2.66 | 0.90 | 33.89 | 2.69 | 1.07 | 39.62 | 36.76 | 28.96 | |
| T074R | 0.54 | 0.05 | 9.92 | 0.56 | 0.03 | 5.99 | 7.96 | 28.96 | |
| T074V | 2.02 | 0.73 | 36.30 | 2.42 | 0.88 | 36.34 | 36.32 | 28.96 | |
| T074W | 0.23 | 0.10 | 42.24 | 0.22 | 0.10 | 43.71 | 42.97 | 35.91 | |
| V075A | 0.41 | 0.16 | 37.74 | 0.52 | 0.20 | 38.34 | 38.04 | 27.69 | |
| V075F | 0.84 | 0.34 | 40.34 | 1.02 | 0.39 | 38.28 | 39.31 | 27.69 | |
| V075N | 1.69 | 0.52 | 30.58 | 1.19 | 0.28 | 23.43 | 27.01 | 35.91 | |
| V075Q | 2.73 | 0.76 | 27.68 | 2.60 | 0.87 | 33.44 | 30.56 | 31.00 | |
| V075R | 1.12 | 0.39 | 34.77 | 1.31 | 0.45 | 34.11 | 34.44 | 27.69 | |
| V075S | 0.30 | 0.03 | 9.26 | 0.28 | 0.01 | 5.02 | 7.14 | 34.21 | |
| V075T | 0.52 | 0.15 | 29.29 | 0.83 | 0.25 | 30.35 | 29.82 | 27.69 | |
| V075Y | 2.11 | 0.63 | 29.72 | 1.87 | 0.55 | 29.61 | 29.67 | 29.23 | |
| I079L | 0.54 | 0.17 | 30.63 | 0.57 | 0.20 | 34.74 | 32.68 | 34.21 | |
| I079V | 0.32 | 0.09 | 28.71 | 0.28 | 0.08 | 29.36 | 29.03 | 34.21 | |
| K082A | 1.17 | 0.49 | 41.98 | 1.05 | 0.32 | 30.67 | 36.33 | 25.91 | |
| K082E | 0.37 | 0.08 | 21.82 | 0.34 | 0.05 | 14.14 | 17.98 | 34.21 | |
| K082G | 0.19 | 0.06 | 31.82 | 0.20 | 0.04 | 19.18 | 25.50 | 33.36 | |
| K082H | 0.66 | 0.21 | 31.11 | 0.60 | 0.15 | 24.27 | 27.69 | 25.91 | |
| K082L | 0.61 | 0.14 | 23.14 | 0.62 | 0.13 | 21.39 | 22.26 | 33.36 | |
| K082M | 1.12 | 0.34 | 30.45 | 1.17 | 0.33 | 28.30 | 29.37 | 31.71 | |
| K082N | 0.23 | 0.07 | 32.75 | 0.17 | 0.06 | 35.21 | 33.98 | 33.36 | |
| K082Q | 0.74 | 0.19 | 25.44 | 0.68 | 0.17 | 25.28 | 25.36 | 33.36 | |
| K082R | 2.33 | 0.57 | 24.49 | 2.26 | 0.57 | 25.08 | 24.78 | 29.23 | |
| K082S | 0.82 | 0.32 | 38.34 | 0.73 | 0.24 | 33.38 | 35.86 | 25.91 | |
| K082T | 1.17 | 0.22 | 19.13 | 1.04 | 0.17 | 16.31 | 17.72 | 31.71 | |
| K082V | 0.64 | 0.23 | 35.30 | 0.48 | 0.20 | 41.59 | 38.44 | 25.91 | |
| K082Y | 0.75 | 0.19 | 25.27 | 0.86 | 0.20 | 23.17 | 24.22 | 31.00 | |
| I083F | 0.81 | 0.08 | 9.99 | 0.98 | 0.03 | 2.91 | 6.45 | 31.71 | |
| I083G | 1.27 | 0.66 | 51.49 | 1.09 | 0.54 | 49.77 | 50.63 | 25.91 | 135 |
| I083H | 0.42 | 0.18 | 43.61 | 0.35 | 0.13 | 36.05 | 39.83 | 25.91 | |
| I083L | 0.80 | 0.11 | 13.88 | 0.88 | 0.10 | 10.83 | 12.35 | 31.71 | |
| I083N | 0.98 | 0.25 | 25.88 | 0.84 | 0.24 | 27.89 | 26.89 | 25.91 | |
| I083Q | 0.29 | 0.22 | 76.55 | 0.32 | 0.19 | 58.80 | 67.67 | 37.07 | 258 |
| I083S | 1.01 | 0.29 | 28.51 | 1.09 | 0.24 | 21.88 | 25.19 | 37.33 | |
| I083T | 1.23 | 0.45 | 36.37 | 0.91 | 0.35 | 38.49 | 37.43 | 25.91 | |
| I083V | 1.65 | 0.87 | 52.90 | 1.63 | 0.78 | 47.98 | 50.44 | 33.02 | 137 |
| S084D | 0.41 | 0.29 | 70.81 | 0.39 | 0.24 | 60.81 | 65.81 | 37.07 | 257 |
| S084E | 0.51 | 0.20 | 38.19 | 0.52 | 0.21 | 40.82 | 39.51 | 33.02 | |
| S084F | 0.43 | 0.18 | 41.95 | 0.44 | 0.16 | 35.63 | 38.79 | 33.02 | |
| S084G | 0.22 | 0.06 | 28.77 | 0.23 | 0.03 | 13.92 | 21.34 | 31.63 | |
| S084H | 0.76 | 0.25 | 32.87 | 0.81 | 0.26 | 31.49 | 32.18 | 33.02 | |
| S084I | 0.75 | 0.26 | 34.67 | 0.79 | 0.21 | 26.07 | 30.37 | 33.02 | |
| S084L | 0.47 | 0.10 | 22.02 | 0.55 | 0.09 | 17.23 | 19.63 | 37.33 | |
| S084N | 1.46 | 0.48 | 32.65 | 1.60 | 0.52 | 32.37 | 32.51 | 31.63 | |
| S084Q | 0.88 | 0.27 | 31.09 | 0.80 | 0.28 | 34.69 | 32.89 | 33.02 | |
| S084R | 0.81 | 0.31 | 37.68 | 0.67 | 0.23 | 34.05 | 35.87 | 31.63 | |
| S084T | 0.65 | 0.16 | 24.16 | 0.78 | 0.12 | 15.64 | 19.90 | 37.33 | |
| S084W | 0.53 | 0.15 | 28.80 | 0.50 | 0.15 | 29.45 | 29.12 | 28.17 | |
| Q086A | 2.54 | 0.90 | 35.51 | 2.04 | 0.63 | 30.64 | 33.08 | 31.00 | |
| Q086D | 2.46 | 2.22 | 90.11 | 1.46 | 0.77 | 52.87 | 71.49 | 32.02 | 206 |
| Q086E | 0.21 | 0.21 | 99.52 | 0.55 | 0.29 | 53.09 | 76.30 | 26.52 | 345 |
| Q086F | 1.86 | 0.49 | 26.14 | 1.60 | 0.43 | 26.84 | 26.49 | 31.00 | |
| Q086G | 0.19 | 0.08 | 38.92 | 0.29 | 0.11 | 40.18 | 39.55 | 22.03 | |
| Q086H | 0.85 | 0.26 | 30.12 | 1.18 | 0.35 | 29.84 | 29.98 | 22.03 | |
| Q086I | 1.99 | 0.59 | 29.41 | 2.60 | 0.58 | 22.36 | 25.89 | 31.00 | |
| Q086K | 0.31 | 0.10 | 30.98 | 0.43 | 0.18 | 41.76 | 36.37 | 32.02 | |
| Q086L | 1.03 | 0.28 | 27.16 | 1.27 | 0.35 | 27.31 | 27.23 | 22.03 | |
| Q086M | 0.60 | 0.20 | 33.78 | 0.48 | 0.10 | 20.21 | 26.99 | 32.02 | |
| Q086N | 1.13 | 0.40 | 35.11 | 0.50 | 0.39 | 76.92 | 56.01 | 32.02 | 141 |
| Q086P | 2.18 | 0.59 | 26.89 | 2.01 | 0.57 | 28.19 | 27.54 | 29.23 | |
| Q086R | 0.66 | 0.32 | 47.84 | 0.66 | 1.03 | 155.95 | 101.90 | 12.70 | 114 |
| Q086S | 1.84 | 0.61 | 33.00 | 3.21 | 1.65 | 51.32 | 42.16 | 12.70 | |
| Q086T | 2.91 | 0.78 | 26.76 | 2.12 | 0.64 | 30.26 | 28.51 | 31.00 | |
| Q086V | 0.53 | 0.09 | 16.65 | 0.55 | 0.34 | 62.16 | 39.40 | 12.70 | |
| Q086W | 0.68 | 0.20 | 29.26 | 0.97 | 0.23 | 23.63 | 26.44 | 22.03 | |
| D087A | 0.18 | 0.01 | 5.72 | 0.46 | 0.14 | 31.27 | 18.50 | 23.18 | |
| D087E | 1.18 | 0.39 | 32.57 | 1.24 | 0.35 | 28.50 | 30.54 | 33.02 | |
| D087G | 1.17 | 0.46 | 39.13 | 1.23 | 0.44 | 36.09 | 37.61 | 36.72 | |
| D087H | 0.32 | 0.08 | 24.81 | 0.82 | 0.34 | 41.94 | 33.37 | 38.36 | |
| D087I | 0.40 | 0.14 | 34.43 | 0.41 | 0.13 | 30.42 | 32.43 | 37.91 | |
| D087M | 0.76 | 0.49 | 64.46 | 0.91 | 0.37 | 41.09 | 52.77 | 36.72 | 143 |
| D087P | 1.10 | 0.36 | 32.74 | 1.15 | 1.22 | 106.23 | 69.49 | 37.00 | 224 |
| D087Q | 1.02 | 0.54 | 53.07 | 1.17 | 0.48 | 40.72 | 46.90 | 36.72 | |
| D087R | 1.12 | 0.57 | 50.74 | 1.05 | 0.48 | 46.21 | 48.47 | 36.72 | |
| D087S | 1.61 | 0.79 | 49.22 | 1.09 | 0.44 | 40.69 | 44.96 | 39.71 | |
| D087T | 0.47 | 0.13 | 27.83 | 0.88 | 0.18 | 20.27 | 24.05 | 37.33 | |
| D087V | 0.46 | 0.26 | 56.64 | 0.52 | 0.24 | 45.87 | 51.26 | 36.72 | 145 |
| D090A | 1.20 | 0.54 | 45.08 | 0.91 | 0.41 | 45.39 | 45.23 | 36.72 | |
| D090E | 0.35 | 0.22 | 64.31 | 0.29 | 0.13 | 44.82 | 54.56 | 39.71 | 146 |
| D090H | 0.91 | 0.47 | 51.23 | 0.85 | 0.37 | 43.58 | 47.41 | 36.72 | |
| D090I | 1.31 | 0.63 | 47.94 | 1.22 | 0.57 | 46.58 | 47.26 | 36.72 | |
| D090K | 1.63 | 0.56 | 34.08 | 1.85 | 0.55 | 29.70 | 31.89 | 26.56 | |
| D090L | 0.53 | 0.21 | 40.40 | 0.51 | 0.19 | 37.50 | 38.95 | 37.65 | |
| D090N | 1.42 | 0.47 | 32.77 | 1.87 | 0.51 | 27.17 | 29.97 | 41.91 | |
| D090Q | 0.74 | 0.28 | 37.40 | 0.91 | 0.26 | 28.54 | 32.97 | 41.91 | |
| D090R | 0.26 | 0.14 | 52.51 | 0.29 | 0.11 | 38.32 | 45.41 | 41.91 | |
| D090S | 0.80 | 0.28 | 34.82 | 0.76 | 0.27 | 36.02 | 35.42 | 26.56 | |
| D090T | 0.11 | 0.08 | 69.74 | 0.18 | 0.09 | 50.14 | 59.94 | 41.91 | 147 |
| D090W | 2.20 | 2.61 | 118.77 | 1.84 | 3.28 | 178.03 | 148.40 | 33.22 | 89 |
| K091A | 0.57 | 0.19 | 33.33 | 0.53 | 0.20 | 37.43 | 35.38 | 33.22 | |
| K091Q | 0.53 | 0.18 | 34.05 | 0.52 | 0.19 | 35.73 | 34.89 | 33.22 | |
| K091R | 0.88 | 0.22 | 25.40 | 0.85 | 0.21 | 24.44 | 24.92 | 33.22 | |
| A092C | 0.78 | 0.16 | 20.98 | 0.67 | 0.16 | 23.24 | 22.11 | 33.22 | |
| A092M | 0.49 | 0.14 | 27.64 | 0.53 | 0.12 | 22.88 | 25.26 | 33.22 | |
| A092T | 0.83 | 0.25 | 29.52 | 0.90 | 0.19 | 20.61 | 25.07 | 33.22 | |
| A092V | 0.42 | 0.37 | 89.68 | 1.01 | 0.33 | 32.69 | 61.18 | 33.22 | 376 |
| K093E | 1.39 | 0.53 | 37.74 | 1.58 | 2.86 | 180.18 | 108.96 | 35.80 | 102 |
| K093F | 0.91 | 0.16 | 17.65 | 0.94 | 0.17 | 17.84 | 17.75 | 31.71 | |
| K093G | 0.81 | 0.13 | 16.31 | 1.18 | 0.61 | 52.02 | 34.16 | 35.80 | |
| K093I | 0.78 | 0.22 | 27.84 | 1.76 | 0.65 | 37.11 | 32.48 | 35.80 | |
| K093M | 1.17 | 0.32 | 27.57 | 3.10 | 1.07 | 34.38 | 30.97 | 35.80 | |
| K093N | 0.97 | 0.27 | 27.31 | 2.35 | 0.76 | 32.43 | 29.87 | 35.80 | |
| K093Q | 1.05 | 0.31 | 29.24 | 2.98 | 0.99 | 33.07 | 31.15 | 35.80 | |
| K093R | 0.36 | 0.15 | 41.96 | 0.94 | 0.41 | 43.07 | 42.51 | 35.80 | |
| K093S | 1.84 | 3.73 | 202.14 | 1.14 | 1.91 | 167.19 | 184.67 | 8.44 | 78 |
| K093T | 2.37 | 0.83 | 34.77 | 1.86 | 0.47 | 25.14 | 29.95 | 8.44 | |
| K093V | 1.01 | 0.36 | 35.54 | 1.42 | 0.56 | 39.08 | 37.31 | 37.00 | |
| K094A | 2.24 | 0.65 | 29.07 | 1.37 | 0.36 | 26.11 | 27.59 | 8.44 | |
| K094D | 1.84 | 0.58 | 31.44 | 1.40 | 0.41 | 29.37 | 30.41 | 8.44 | |
| K094E | 1.89 | 0.61 | 32.00 | 1.11 | 0.34 | 30.07 | 31.04 | 8.44 | |
| K094F | 0.66 | 0.19 | 29.38 | 0.36 | 0.11 | 29.99 | 29.68 | 8.44 | |
| K094H | 0.38 | 0.16 | 42.50 | 0.22 | 0.09 | 38.43 | 40.46 | 8.44 | |
| K094M | 1.53 | 0.59 | 38.79 | 0.89 | 0.26 | 28.94 | 33.86 | 8.44 | |
| K094N | 0.24 | 0.09 | 36.57 | 0.22 | 0.20 | 91.38 | 63.98 | 11.35 | 335 |
| K094Q | 0.82 | 0.23 | 28.18 | 0.67 | 0.20 | 29.10 | 28.64 | 11.35 | |
| K094R | 0.56 | 0.16 | 28.39 | 0.52 | 0.16 | 29.94 | 29.17 | 11.35 | |
| K094S | 0.94 | 0.25 | 26.05 | 0.80 | 0.18 | 22.89 | 24.47 | 11.35 | |
| K094T | 0.20 | 0.06 | 31.84 | 0.15 | 0.04 | 27.91 | 29.87 | 11.35 | |
| I096L | 0.26 | 0.06 | 24.39 | 0.23 | 0.04 | 19.47 | 21.93 | 11.35 | |
| T097C | 0.34 | 0.15 | 44.08 | 0.47 | 0.18 | 37.99 | 41.04 | 28.16 | |
| T097D | 1.26 | 0.39 | 31.06 | 1.57 | 0.45 | 28.39 | 29.73 | 28.16 | |
| T097E | 0.86 | 0.40 | 46.23 | 0.54 | 0.35 | 65.18 | 55.70 | 33.76 | 153 |
| T097F | 1.15 | 1.49 | 129.56 | 2.98 | 1.32 | 44.38 | 86.97 | 28.16 | 165 |
| T097G | 2.03 | 0.68 | 33.52 | 4.72 | 2.75 | 58.21 | 45.87 | 15.00 | |
| T097I | 1.12 | 0.41 | 36.27 | 1.86 | 0.92 | 49.57 | 42.92 | 15.00 | |
| T097L | 2.18 | 0.81 | 37.02 | 3.14 | 1.43 | 45.73 | 41.37 | 15.00 | |
| T097N | 3.09 | 0.89 | 28.89 | 4.81 | 1.68 | 34.96 | 31.93 | 15.00 | |
| T097Q | 3.01 | 0.82 | 27.16 | 4.61 | 1.78 | 38.68 | 32.92 | 15.00 | |
| T097S | 2.12 | 0.60 | 28.12 | 3.06 | 0.95 | 31.11 | 29.61 | 15.00 | |
| T097Y | 1.33 | 0.42 | 31.56 | 2.16 | 0.85 | 39.08 | 35.32 | 15.00 | |
| F098H | 4.75 | 2.10 | 44.17 | 2.63 | 0.80 | 30.38 | 37.27 | 29.98 | |
| F098I | 0.89 | 0.28 | 31.48 | 0.70 | 0.14 | 19.60 | 25.54 | 29.98 | |
| F098L | 1.17 | 0.41 | 34.80 | 0.87 | 0.18 | 20.35 | 27.58 | 29.98 | |
| F098M | 5.47 | 3.34 | 61.03 | 3.84 | 2.53 | 65.85 | 63.44 | 29.23 | 295 |
| F098R | 2.09 | 0.55 | 26.07 | 2.64 | 0.82 | 30.89 | 28.48 | 37.00 | |
| F098S | 0.37 | 0.22 | 58.22 | 0.33 | 0.09 | 27.60 | 42.91 | 29.98 | |
| F098W | 1.01 | 0.16 | 16.09 | 0.89 | 0.17 | 19.17 | 17.63 | 4.17 | |
| Y099R | 0.85 | 0.22 | 25.25 | 0.85 | 0.58 | 68.37 | 46.81 | 24.78 | |
| Y099S | 0.23 | 0.18 | 79.18 | 0.23 | 0.16 | 69.98 | 74.58 | 37.23 | 225 |
| V102E | 2.73 | 0.94 | 34.44 | 2.15 | 0.58 | 26.77 | 30.60 | 29.98 | |
| T306D | 0.24 | 0.11 | 45.96 | 0.30 | 0.30 | 102.19 | 74.08 | 32.76 | 256 |
| V102K | 4.11 | 1.32 | 32.00 | 2.56 | 0.86 | 33.63 | 32.82 | 29.98 | |
| V102N | 7.15 | 4.61 | 64.52 | 5.59 | 2.67 | 47.82 | 56.17 | 29.98 | 158 |
| V102Q | 2.33 | 0.85 | 36.62 | 2.58 | 0.76 | 29.41 | 33.02 | 37.65 | |
| V102R | 2.30 | 0.61 | 26.68 | 2.17 | 0.47 | 21.74 | 24.21 | 37.65 | |
| V102S | 1.33 | 0.43 | 32.13 | 1.28 | 0.30 | 23.46 | 27.80 | 37.65 | |
| V102W | 1.32 | 0.44 | 33.02 | 1.62 | 0.57 | 35.17 | 34.09 | 37.00 | |
| N104A | 1.22 | 0.51 | 41.59 | 1.31 | 0.54 | 41.62 | 41.60 | 21.42 | |
| N104G | 0.34 | 0.07 | 20.79 | 1.05 | 0.42 | 39.64 | 30.21 | 37.00 | |
| N104K | 1.22 | 0.41 | 33.51 | 1.27 | 0.34 | 27.15 | 30.33 | 37.65 | |
| N104R | 1.09 | 0.38 | 34.68 | 1.02 | 0.25 | 24.02 | 29.35 | 37.65 | |
| N104T | 0.73 | 0.19 | 26.45 | 2.24 | 0.94 | 41.97 | 34.21 | 37.00 | |
| L105G | 0.46 | 0.18 | 39.04 | 0.56 | 0.21 | 37.79 | 38.41 | 37.23 | |
| L105I | 0.32 | 0.16 | 50.47 | 0.48 | 0.31 | 65.06 | 57.77 | 24.78 | 159 |
| L105Q | 0.17 | 0.09 | 55.29 | 0.21 | 0.06 | 28.19 | 41.74 | 37.65 | |
| L105R | 2.36 | 0.69 | 29.28 | 3.76 | 3.17 | 84.29 | 56.78 | 24.78 | 160 |
| L105W | 0.20 | 0.07 | 33.82 | 0.19 | 0.13 | 67.99 | 50.91 | 24.78 | 161 |
| M107F | 0.91 | 0.36 | 39.84 | 0.99 | 0.40 | 40.67 | 40.25 | 21.42 | |
| I110V | 0.72 | 0.25 | 34.15 | 0.73 | 0.25 | 33.38 | 33.76 | 31.87 | |
| E114G | 0.17 | 0.10 | 60.84 | 0.12 | 0.09 | 73.82 | 67.33 | 37.02 | 280 |
| T118L | 1.08 | 0.28 | 26.30 | 1.04 | 0.24 | 23.24 | 24.77 | 21.42 | |
| T118M | 0.17 | 0.09 | 54.76 | 0.11 | 0.09 | 77.78 | 66.27 | 37.02 | 281 |
| A120H | 1.41 | 0.38 | 26.68 | 1.10 | 0.30 | 27.67 | 27.17 | 31.87 | |
| A120I | 1.77 | 0.34 | 19.51 | 1.58 | 0.31 | 19.32 | 19.42 | 31.87 | |
| A120L | 0.35 | 0.08 | 23.82 | 0.86 | 0.11 | 13.32 | 18.57 | 37.33 | |
| A120R | 1.05 | 0.16 | 15.31 | 1.16 | 0.17 | 14.87 | 15.09 | 31.87 | |
| A120S | 0.16 | 0.17 | 108.54 | 0.23 | 0.15 | 65.86 | 87.20 | 21.42 | 297 |
| A120W | 0.90 | 0.17 | 18.32 | 0.96 | 0.14 | 14.54 | 16.43 | 27.61 | |
| A120Y | 0.24 | 0.16 | 66.25 | 0.33 | 0.10 | 29.88 | 48.06 | 27.61 | |
| K124R | 1.15 | 0.36 | 31.70 | 1.07 | 0.27 | 25.59 | 28.64 | 27.61 | |
| D127A | 0.75 | 0.11 | 14.13 | 0.70 | 0.07 | 10.20 | 12.17 | 39.93 | |
| D127E | 1.55 | 0.28 | 18.04 | 1.43 | 0.25 | 17.50 | 17.77 | 27.61 | |
| D127G | 0.91 | 0.15 | 16.53 | 1.31 | 0.19 | 14.25 | 15.39 | 27.61 | |
| D127H | 1.95 | 0.28 | 14.54 | 1.74 | 0.24 | 13.63 | 14.08 | 27.61 | |
| D127L | 0.83 | 0.11 | 13.12 | 1.12 | 0.15 | 13.20 | 13.16 | 37.02 | |
| D127M | 0.74 | 0.11 | 14.92 | 1.04 | 0.13 | 12.74 | 13.83 | 37.02 | |
| D127N | 2.89 | 0.29 | 10.04 | 2.47 | 0.23 | 9.12 | 9.58 | 27.61 | |
| D127Q | 1.91 | 0.12 | 6.19 | 2.18 | 0.15 | 6.98 | 6.59 | 30.27 | |
| D127S | 1.65 | 0.18 | 11.11 | 1.27 | 0.12 | 9.77 | 10.44 | 39.93 | |
| D127T | 3.14 | 0.19 | 5.95 | 3.52 | 0.20 | 5.82 | 5.88 | 30.27 | |
| D127W | 0.73 | 0.06 | 8.01 | 0.32 | 0.04 | 11.36 | 9.69 | 39.93 | |
| V128G | 1.01 | 0.10 | 9.39 | 1.04 | 0.09 | 8.59 | 8.99 | 30.27 | |
| V128I | 1.60 | 0.42 | 26.53 | 1.72 | 0.45 | 26.16 | 26.34 | 30.27 | |
| V128K | 1.87 | 0.15 | 7.85 | 1.91 | 0.14 | 7.53 | 7.69 | 30.27 | |
| V128Q | 0.90 | 0.09 | 10.40 | 0.92 | 0.06 | 6.64 | 8.52 | 30.27 | |
| V128S | 1.08 | 0.10 | 9.62 | 1.14 | 0.10 | 8.89 | 9.25 | 30.27 | |
| V128W | 1.34 | 0.15 | 11.23 | 2.19 | 0.36 | 16.54 | 13.89 | 30.27 | |
| N131C | 0.80 | 0.35 | 43.44 | 2.04 | 1.64 | 80.51 | 61.98 | 31.39 | 352 |
| N131G | 0.97 | 0.16 | 16.32 | 1.12 | 0.17 | 15.04 | 15.68 | 37.02 | |
| N131H | 1.95 | 0.65 | 33.37 | 3.04 | 1.36 | 44.90 | 39.13 | 31.39 | |
| N131I | 0.12 | 0.08 | 65.69 | 0.21 | 0.06 | 27.47 | 46.58 | 37.33 | |
| N131L | 0.23 | 0.19 | 82.09 | 0.25 | 0.22 | 87.82 | 84.95 | 31.39 | 207 |
| N131M | 0.96 | 0.35 | 36.43 | 1.68 | 0.65 | 38.56 | 37.50 | 31.39 | |
| N131Q | 3.58 | 1.34 | 37.49 | 3.84 | 0.86 | 22.47 | 29.98 | 37.02 | |
| N131R | 1.82 | 0.66 | 36.38 | 2.48 | 1.23 | 49.78 | 43.08 | 31.39 | |
| N131S | 0.61 | 0.25 | 41.42 | 0.74 | 0.30 | 39.84 | 40.63 | 31.39 | |
| N131T | 1.39 | 0.57 | 41.47 | 2.31 | 0.99 | 42.78 | 42.12 | 31.39 | |
| N131V | 0.93 | 0.30 | 31.57 | 1.26 | 0.29 | 22.68 | 27.13 | 37.02 | |
| N131Y | 3.65 | 0.91 | 24.85 | 2.40 | 0.34 | 14.34 | 19.59 | 34.80 | |
| R132A | 0.32 | 0.32 | 100.00 | 0.36 | 0.36 | 100.00 | 100.00 | 21.42 | 299 |
| R132C | 0.24 | 0.12 | 50.10 | 0.34 | 0.17 | 51.48 | 50.79 | 21.42 | 168 |
| R132F | 0.66 | 0.17 | 25.77 | 0.46 | 0.10 | 20.54 | 23.16 | 34.80 | |
| R132K | 2.16 | 0.77 | 35.84 | 1.23 | 0.27 | 22.04 | 28.94 | 34.80 | |
| R132Q | 1.19 | 0.29 | 24.45 | 1.14 | 0.27 | 23.30 | 23.88 | 21.42 | |
| R132V | 1.69 | 0.44 | 26.09 | 0.92 | 0.17 | 18.12 | 22.11 | 34.80 | |
| I134L | 0.71 | 0.22 | 31.43 | 0.65 | 0.15 | 22.48 | 26.96 | 39.93 | |
| I134T | 2.01 | 0.26 | 12.93 | 1.16 | 0.06 | 4.89 | 8.91 | 34.80 | |
| E135A | 4.14 | 1.11 | 26.81 | 4.34 | 1.16 | 26.78 | 26.79 | 30.48 | |
| E135C | 0.24 | 0.08 | 35.02 | 0.61 | 0.11 | 17.26 | 26.14 | 37.33 | |
| E135K | 0.22 | 0.08 | 33.41 | 0.18 | 0.06 | 33.52 | 33.46 | 30.48 | |
| E135L | 0.71 | 0.14 | 20.10 | 0.64 | 0.15 | 24.06 | 22.08 | 30.48 | |
| E135N | 0.46 | 0.12 | 25.27 | 0.46 | 0.09 | 19.09 | 22.18 | 30.48 | |
| E135Q | 0.27 | 0.22 | 83.93 | 0.98 | 0.28 | 29.12 | 56.52 | 26.52 | 169 |
| E135R | 1.18 | 0.12 | 10.05 | 1.19 | 0.10 | 8.00 | 9.02 | 30.48 | |
| E135S | 0.23 | 0.07 | 32.53 | 0.20 | 0.06 | 31.25 | 31.89 | 30.48 | |
| L136A | 3.46 | 1.33 | 38.57 | 1.84 | 0.42 | 22.51 | 30.54 | 31.71 | |
| L136C | 1.68 | 0.37 | 21.91 | 1.81 | 0.37 | 20.40 | 21.15 | 25.22 | |
| L136H | 2.32 | 0.73 | 31.30 | 2.61 | 0.79 | 30.22 | 30.76 | 21.42 | |
| L136M | 1.33 | 0.40 | 30.04 | 1.23 | 0.33 | 26.97 | 28.50 | 25.22 | |
| L136Q | 0.38 | 0.13 | 33.38 | 0.24 | 0.05 | 20.08 | 26.73 | 39.93 | |
| L136T | 0.46 | 0.18 | 38.97 | 0.38 | 0.14 | 37.35 | 38.16 | 25.22 | |
| V137A | 0.92 | 0.23 | 24.58 | 0.75 | 0.22 | 28.74 | 26.66 | 25.22 | |
| V137C | 2.32 | 0.44 | 19.16 | 1.81 | 0.32 | 17.62 | 18.39 | 31.00 | |
| V137L | 0.70 | 0.18 | 25.16 | 0.90 | 0.24 | 26.11 | 25.63 | 24.78 | |
| V137S | 0.23 | 0.08 | 34.06 | 0.24 | 0.08 | 31.08 | 32.57 | 25.22 | |
| V137T | 2.36 | 0.76 | 32.29 | 1.83 | 0.35 | 18.93 | 25.61 | 25.22 | |
| Q138A | 0.25 | 0.04 | 16.57 | 0.30 | 0.04 | 14.36 | 15.46 | 27.67 | |
| Q138C | 0.25 | 0.12 | 50.00 | 0.24 | 0.10 | 41.53 | 45.76 | 30.53 | |
| Q138I | 0.82 | 0.22 | 26.78 | 0.81 | 0.16 | 20.14 | 23.46 | 27.67 | |
| Q138L | 0.27 | 0.11 | 41.48 | 0.24 | 0.07 | 30.11 | 35.79 | 27.67 | |
| Q138M | 0.47 | 0.16 | 34.89 | 0.59 | 0.25 | 43.06 | 38.98 | 24.78 | |
| Q138R | 0.60 | 0.20 | 34.11 | 0.54 | 0.11 | 20.17 | 27.14 | 27.67 | |
| Q138S | 0.16 | 0.05 | 30.22 | 0.19 | 0.00 | 0.00 | 15.11 | 27.67 | |
| Q138W | 0.20 | 0.13 | 64.74 | 0.25 | 0.19 | 78.57 | 71.65 | 29.57 | 333 |
| Q138Y | 0.61 | 0.11 | 17.86 | 0.83 | 0.53 | 63.72 | 40.79 | 32.16 | |
| Q139A | 0.82 | 0.09 | 10.87 | 0.82 | 0.11 | 13.31 | 12.09 | 31.59 | |
| Q139C | 0.96 | 0.11 | 11.56 | 1.04 | 0.31 | 30.25 | 20.90 | 29.59 | |
| Q139D | 0.92 | 0.00 | 0.00 | 1.05 | 0.03 | 2.44 | 1.22 | 29.59 | |
| Q139E | 0.72 | 0.11 | 15.21 | 0.79 | 0.00 | 0.00 | 7.61 | 29.59 | |
| Q139F | 0.56 | 0.10 | 18.27 | 0.60 | 0.07 | 11.59 | 14.93 | 29.59 | |
| Q139G | 0.25 | 0.04 | 17.07 | 0.27 | 0.06 | 22.22 | 19.65 | 29.59 | |
| Q139H | 1.05 | 0.35 | 33.78 | 1.37 | 0.22 | 16.39 | 25.08 | 24.78 | |
| Q139K | 1.65 | 0.43 | 26.37 | 2.15 | 0.48 | 22.23 | 24.30 | 29.59 | |
| Q139M | 0.14 | 0.02 | 10.95 | 0.22 | 0.00 | 0.00 | 5.47 | 29.59 | |
| Q139R | 1.02 | 1.55 | 152.26 | 1.39 | 0.40 | 28.81 | 90.54 | 24.78 | 144 |
| Q139S | 0.15 | 0.01 | 7.72 | 0.15 | 0.01 | 9.09 | 8.40 | 34.14 | |
| Q139T | 1.27 | 0.28 | 21.89 | 1.59 | 0.37 | 23.35 | 22.62 | 34.14 | |
| Q139V | 0.30 | 0.31 | 100.99 | 0.55 | 0.30 | 55.31 | 78.15 | 38.05 | 108 |
| Q140A | 1.66 | 0.40 | 23.75 | 1.71 | 0.49 | 28.33 | 26.04 | 34.14 | |
| Q140D | 1.15 | 0.23 | 19.84 | 1.74 | 0.39 | 22.19 | 21.01 | 34.14 | |
| Q140F | 1.21 | 0.24 | 19.82 | 1.64 | 0.35 | 21.18 | 20.50 | 34.14 | |
| Q140G | 1.77 | 0.46 | 26.17 | 2.26 | 0.50 | 21.90 | 24.03 | 34.14 | |
| Q140H | 4.85 | 2.41 | 49.73 | 2.61 | 0.98 | 37.45 | 43.59 | 28.36 | |
| Q140I | 2.10 | 0.54 | 25.65 | 1.41 | 0.22 | 15.65 | 20.65 | 28.36 | |
| Q140K | 1.65 | 0.63 | 38.37 | 1.73 | 0.58 | 33.65 | 36.01 | 20.67 | |
| Q140M | 0.81 | 0.29 | 35.50 | 0.95 | 0.32 | 34.05 | 34.78 | 20.67 | |
| Q140R | 2.51 | 1.08 | 43.00 | 1.98 | 0.65 | 32.67 | 37.83 | 28.36 | |
| Q140V | 2.50 | 0.59 | 23.72 | 1.65 | 0.34 | 20.70 | 22.21 | 28.36 | |
| Q140W | 1.92 | 0.40 | 20.94 | 1.11 | 0.15 | 13.67 | 17.30 | 28.36 | |
| Q140Y | 2.79 | 1.75 | 62.72 | 1.03 | 0.28 | 27.47 | 45.09 | 28.36 | |
| N141A | 0.34 | 0.15 | 43.86 | 0.40 | 0.21 | 52.74 | 48.30 | 30.39 | |
| N141D | 0.34 | 0.13 | 37.83 | 0.46 | 0.10 | 21.98 | 29.91 | 40.82 | |
| N141E | 0.74 | 0.27 | 36.59 | 0.69 | 0.35 | 51.04 | 43.81 | 38.36 | |
| N141F | 0.96 | 0.36 | 37.32 | 0.90 | 0.34 | 38.26 | 37.79 | 30.38 | |
| N141G | 0.56 | 0.21 | 37.21 | 0.54 | 0.19 | 35.39 | 36.30 | 30.38 | |
| N141H | 1.16 | 0.44 | 38.35 | 1.17 | 0.51 | 43.88 | 41.12 | 30.38 | |
| N141M | 0.17 | 0.20 | 118.21 | 0.20 | 0.22 | 110.95 | 114.58 | 36.07 | 163 |
| N141Q | 0.27 | 0.14 | 53.77 | 0.28 | 0.13 | 46.57 | 50.17 | 30.38 | 174 |
| N141R | 0.35 | 0.11 | 30.13 | 0.29 | 0.09 | 29.84 | 29.99 | 30.38 | |
| N141S | 2.01 | 0.65 | 32.33 | 1.91 | 0.60 | 31.45 | 31.89 | 30.38 | |
| N141T | 0.56 | 0.29 | 51.25 | 0.56 | 0.25 | 44.40 | 47.83 | 36.07 | |
| N141V | 0.89 | 0.12 | 14.00 | 0.91 | 0.09 | 9.48 | 11.74 | 40.82 | |
| N141W | 0.72 | 0.41 | 57.40 | 0.69 | 0.41 | 58.82 | 58.11 | 36.07 | 175 |
| N369H | 2.70 | 4.20 | 155.73 | 3.74 | 1.63 | 43.69 | 99.71 | 39.44 | 116 |
| V142C | 3.33 | 1.34 | 40.31 | 3.00 | 1.11 | 36.91 | 38.61 | 39.15 | |
| V142D | 0.73 | 0.28 | 38.28 | 0.81 | 0.27 | 33.11 | 35.69 | 39.15 | |
| V142E | 0.21 | 0.11 | 50.24 | 0.19 | 0.08 | 43.38 | 46.81 | 39.15 | |
| V142G | 1.53 | 0.57 | 37.45 | 1.23 | 0.40 | 32.81 | 35.13 | 39.15 | |
| V142H | 3.28 | 0.98 | 29.89 | 2.50 | 0.72 | 28.88 | 29.39 | 39.15 | |
| V142I | 2.16 | 0.65 | 30.22 | 1.82 | 0.49 | 26.67 | 28.45 | 39.15 | |
| V142K | 3.24 | 1.21 | 37.46 | 2.63 | 0.81 | 30.82 | 34.14 | 39.15 | |
| V142L | 1.77 | 0.50 | 27.95 | 1.40 | 0.41 | 29.02 | 28.49 | 32.77 | |
| V142M | 1.35 | 0.48 | 35.75 | 1.53 | 0.52 | 34.03 | 34.89 | 24.78 | |
| V142N | 3.17 | 0.80 | 25.30 | 2.45 | 0.54 | 22.15 | 23.72 | 32.77 | |
| V142Q | 0.21 | 0.12 | 56.12 | 0.17 | 0.10 | 59.36 | 57.74 | 32.77 | 177 |
| V142R | 3.44 | 1.02 | 29.62 | 3.22 | 0.87 | 26.96 | 28.29 | 32.77 | |
| V142S | 2.22 | 0.89 | 40.13 | 3.03 | 0.86 | 28.40 | 34.27 | 32.77 | |
| V142T | 3.63 | 1.78 | 49.15 | 3.61 | 1.16 | 32.24 | 40.70 | 32.77 | |
| Q143C | 4.23 | 1.84 | 43.50 | 4.14 | 1.70 | 41.15 | 42.32 | 38.82 | |
| Q143E | 1.99 | 0.62 | 31.37 | 1.57 | 0.47 | 29.90 | 30.63 | 38.82 | |
| Q143F | 0.33 | 0.13 | 40.92 | 0.33 | 0.13 | 40.92 | 40.92 | 39.93 | |
| Q143G | 0.40 | 0.20 | 49.56 | 0.46 | 0.22 | 47.49 | 48.53 | 37.23 | |
| Q143I | 1.05 | 0.52 | 49.47 | 1.19 | 0.51 | 42.71 | 46.09 | 36.07 | |
| F029H | 0.19 | 0.12 | 63.52 | 0.17 | 0.12 | 68.19 | 65.86 | 30.73 | 267 |
| Q143L | 3.60 | 1.12 | 31.16 | 3.06 | 0.95 | 30.88 | 31.02 | 38.82 | |
| Q143N | 1.28 | 0.49 | 38.50 | 1.12 | 0.40 | 35.87 | 37.19 | 38.82 | |
| Q143V | 2.67 | 0.83 | 31.01 | 2.08 | 0.66 | 31.55 | 31.28 | 38.82 | |
| R311G | 0.46 | 0.29 | 62.43 | 0.54 | 0.32 | 58.39 | 60.41 | 24.30 | 378 |
| L144R | 3.14 | 0.95 | 30.33 | 2.68 | 0.84 | 31.36 | 30.85 | 38.82 | |
| L144T | 2.93 | 1.07 | 36.56 | 2.94 | 0.81 | 27.69 | 32.12 | 38.82 | |
| L144W | 1.24 | 0.32 | 25.82 | 1.23 | 0.29 | 23.81 | 24.82 | 38.36 | |
| S145C | 0.44 | 0.13 | 28.86 | 0.38 | 0.11 | 29.56 | 29.21 | 31.06 | |
| S145D | 0.34 | 0.15 | 44.85 | 0.27 | 0.12 | 44.63 | 44.74 | 31.06 | |
| S145E | 0.83 | 0.23 | 28.25 | 0.79 | 0.18 | 22.21 | 25.23 | 31.06 | |
| S145P | 0.41 | 0.18 | 44.61 | 0.40 | 0.15 | 37.39 | 41.00 | 31.06 | |
| S145R | 3.81 | 0.70 | 18.37 | 3.69 | 0.68 | 18.37 | 18.37 | 24.42 | |
| L146E | 1.05 | 0.24 | 22.45 | 0.94 | 0.19 | 20.06 | 21.26 | 24.42 | |
| L146G | 1.51 | 0.22 | 14.58 | 1.35 | 0.14 | 10.30 | 12.44 | 24.42 | |
| L146V | 0.18 | 0.15 | 82.07 | 0.17 | 0.13 | 75.29 | 78.68 | 22.16 | 306 |
| L146Y | 0.69 | 0.30 | 42.70 | 0.76 | 0.33 | 43.15 | 42.93 | 28.04 | |
| T147G | 0.58 | 0.34 | 58.71 | 0.58 | 0.26 | 44.29 | 51.50 | 24.42 | 181 |
| A048G | 0.27 | 0.26 | 97.55 | 0.33 | 0.22 | 65.36 | 81.46 | 28.04 | 241 |
| T147M | 0.16 | 0.15 | 93.08 | 0.17 | 0.16 | 97.91 | 95.50 | 22.16 | 308 |
| T147Q | 2.26 | 0.72 | 31.73 | 2.48 | 0.67 | 27.13 | 29.43 | 24.42 | |
| T147R | 5.79 | 2.37 | 41.00 | 4.17 | 1.23 | 29.41 | 35.20 | 24.42 | |
| E148C | 0.17 | 0.17 | 103.59 | 0.18 | 0.16 | 90.91 | 97.25 | 22.16 | 309 |
| E148H | 0.17 | 0.13 | 76.33 | 0.13 | 0.15 | 108.21 | 92.27 | 22.16 | 310 |
| E148K | 0.14 | 0.11 | 80.37 | 0.14 | 0.11 | 80.87 | 80.62 | 22.16 | 311 |
| E148L | 0.36 | 0.17 | 46.78 | 0.40 | 0.17 | 41.71 | 44.24 | 38.36 | |
| E148Q | 1.16 | 0.36 | 30.83 | 1.37 | 0.29 | 21.24 | 26.03 | 25.15 | |
| E148R | 2.62 | 0.51 | 19.60 | 2.79 | 0.50 | 17.83 | 18.71 | 28.04 | |
| E148S | 0.63 | 0.23 | 35.91 | 0.75 | 0.19 | 25.76 | 30.84 | 38.36 | |
| E148Y | 0.67 | 0.11 | 16.13 | 0.62 | 0.12 | 18.67 | 17.40 | 25.15 | |
| A149C | 0.93 | 0.19 | 20.19 | 0.99 | 0.15 | 15.32 | 17.76 | 25.15 | |
| A149M | 1.18 | 0.18 | 15.43 | 1.29 | 0.19 | 14.70 | 15.07 | 25.15 | |
| A149Q | 2.66 | 0.57 | 21.60 | 2.27 | 0.51 | 22.41 | 22.00 | 30.69 | |
| A149R | 1.77 | 0.21 | 11.60 | 1.55 | 0.22 | 14.47 | 13.04 | 30.69 | |
| A149T | 0.82 | 0.19 | 23.51 | 0.86 | 0.17 | 19.97 | 21.74 | 30.69 | |
| A149V | 1.64 | 0.31 | 19.03 | 1.46 | 0.32 | 22.26 | 20.65 | 30.69 | |
| T150A | 0.78 | 0.06 | 7.35 | 0.69 | 0.06 | 8.62 | 7.98 | 31.59 | |
| T150C | 0.77 | 0.07 | 8.97 | 0.71 | 0.06 | 8.76 | 8.87 | 31.59 | |
| T150D | 1.15 | 0.13 | 11.48 | 1.09 | 0.12 | 10.65 | 11.06 | 31.59 | |
| T150E | 0.41 | 0.05 | 10.94 | 0.38 | 0.03 | 8.52 | 9.73 | 31.59 | |
| T150F | 0.39 | 0.03 | 8.63 | 0.42 | 0.02 | 5.71 | 7.17 | 31.59 | |
| T150G | 1.17 | 0.29 | 24.61 | 1.45 | 0.33 | 22.60 | 23.61 | 22.03 | |
| T150L | 0.20 | 0.13 | 63.84 | 0.17 | 0.17 | 95.93 | 79.89 | 27.54 | 372 |
| T150N | 0.51 | 0.04 | 6.94 | 0.49 | 0.01 | 2.46 | 4.70 | 31.59 | |
| T150R | 0.90 | 0.07 | 7.29 | 0.99 | 0.10 | 9.60 | 8.44 | 31.59 | |
| T150S | 0.86 | 0.28 | 32.96 | 0.99 | 0.34 | 33.91 | 33.44 | 36.75 | |
| T150W | 0.14 | 0.03 | 22.70 | 0.21 | 0.10 | 47.49 | 35.09 | 36.75 | |
| T150Y | 0.48 | 0.21 | 44.20 | 1.38 | 0.49 | 35.08 | 39.64 | 36.75 | |
| E151A | 1.25 | 0.44 | 34.82 | 1.30 | 0.29 | 22.32 | 28.57 | 14.96 | |
| E151C | 0.34 | 0.22 | 64.70 | 0.47 | 0.09 | 19.98 | 42.34 | 14.96 | |
| E151G | 0.39 | 0.12 | 29.99 | 0.38 | 0.09 | 22.66 | 26.32 | 14.96 | |
| E151H | 1.97 | 0.52 | 26.32 | 2.04 | 0.42 | 20.84 | 23.58 | 14.96 | |
| E151K | 0.22 | 0.01 | 6.05 | 0.24 | 0.03 | 14.14 | 10.09 | 14.96 | |
| E151M | 0.33 | 0.13 | 37.54 | 0.25 | 0.01 | 2.37 | 19.95 | 14.96 | |
| E151N | 1.07 | 0.16 | 15.15 | 1.02 | 0.18 | 17.14 | 16.14 | 3.38 | |
| E151Q | 0.10 | 0.12 | 116.75 | 0.34 | 0.14 | 39.82 | 78.28 | 38.36 | 138 |
| E151R | 0.26 | 0.00 | 0.00 | 0.19 | 0.00 | 0.00 | 0.00 | 3.38 | |
| E151S | 1.23 | 0.18 | 14.78 | 1.13 | 0.17 | 14.89 | 14.83 | 3.38 | |
| E151T | 0.67 | 0.09 | 13.80 | 0.74 | 0.05 | 6.12 | 9.96 | 3.38 | |
| E151V | 0.52 | 0.06 | 11.37 | 0.51 | 0.01 | 2.74 | 7.05 | 3.38 | |
| E151W | 1.46 | 0.23 | 15.69 | 1.65 | 0.15 | 8.93 | 12.31 | 3.38 | |
| E151Y | 1.43 | 0.23 | 16.14 | 1.73 | 0.18 | 10.52 | 13.33 | 3.38 | |
| K152A | 0.66 | 0.44 | 67.48 | 1.16 | 0.51 | 44.36 | 55.92 | 54.87 | 189 |
| K152C | 0.82 | 0.28 | 34.27 | 0.64 | 0.21 | 33.52 | 33.89 | 28.17 | |
| K152F | 0.83 | 0.20 | 23.51 | 0.80 | 0.20 | 24.39 | 23.95 | 16.94 | |
| K152I | 0.75 | 0.61 | 81.59 | 0.89 | 0.59 | 66.95 | 74.27 | 54.87 | 199 |
| K152M | 0.34 | 0.45 | 131.63 | 0.45 | 0.40 | 88.41 | 110.02 | 54.87 | 106 |
| K152R | 0.27 | 0.14 | 51.58 | 0.37 | 0.11 | 28.65 | 40.12 | 24.88 | |
| K152T | 0.12 | 0.31 | 269.40 | 0.14 | 0.23 | 160.70 | 215.05 | 54.87 | 107 |
| K152V | 0.98 | 0.37 | 37.21 | 1.11 | 0.33 | 30.20 | 33.71 | 16.94 | |
| K154R | 1.06 | 1.02 | 96.03 | 1.23 | 1.01 | 82.19 | 89.11 | 66.14 | 152 |
| Q155A | 0.87 | 0.99 | 113.56 | 0.97 | 0.95 | 97.48 | 105.52 | 66.14 | 112 |
| R060K | 1.04 | 0.59 | 56.16 | 1.27 | 0.46 | 36.35 | 46.26 | 26.81 | 385 |
| Q155F | 0.63 | 0.80 | 127.25 | 0.60 | 0.77 | 129.22 | 128.24 | 66.14 | 93 |
| Q155H | 3.15 | 1.86 | 59.05 | 6.16 | 4.65 | 75.49 | 67.27 | 32.29 | 236 |
| Q155K | 3.89 | 1.30 | 33.33 | 4.20 | 1.44 | 34.19 | 33.76 | 32.29 | |
| Q155L | 0.51 | 0.71 | 138.94 | 0.60 | 0.71 | 119.04 | 128.99 | 66.14 | 91 |
| Q155M | 2.23 | 0.78 | 34.89 | 2.14 | 0.66 | 30.76 | 32.83 | 32.29 | |
| Q155R | 1.72 | 1.30 | 75.66 | 1.79 | 1.26 | 70.39 | 73.02 | 66.14 | 205 |
| Q155S | 1.57 | 0.88 | 55.98 | 0.23 | 0.17 | 74.46 | 65.22 | 29.93 | 264 |
| Q155T | 0.62 | 0.25 | 40.55 | 1.12 | 0.52 | 46.72 | 43.64 | 29.93 | |
| Q155V | 1.40 | 0.74 | 53.05 | 1.19 | 0.59 | 49.41 | 51.23 | 29.93 | 201 |
| Q155W | 1.47 | 0.72 | 48.81 | 1.58 | 0.68 | 42.73 | 45.77 | 34.46 | |
| E156A | 1.04 | 0.53 | 51.30 | 0.32 | 0.21 | 64.41 | 57.86 | 29.93 | 202 |
| E156C | 0.63 | 0.36 | 57.78 | 0.43 | 0.23 | 53.52 | 55.65 | 29.93 | 203 |
| E156M | 0.67 | 0.29 | 43.47 | 0.67 | 0.29 | 43.22 | 43.34 | 29.93 | |
| E156Q | 1.63 | 0.91 | 55.52 | 1.39 | 0.86 | 61.75 | 58.64 | 29.93 | 204 |
| E156S | 0.00 | 1.28 | #DIV/0! | 0.81 | 2.75 | 340.88 | #DIV/0! | 29.93 | |
| E158A | 0.36 | 0.09 | 24.83 | 0.44 | 0.07 | 16.13 | 20.48 | 20.63 | |
| E158F | 0.92 | 0.19 | 21.21 | 0.91 | 0.24 | 26.23 | 23.72 | 24.02 | |
| E158H | 0.18 | 0.10 | 54.78 | 0.19 | 0.20 | 101.55 | 78.16 | 40.82 | 75 |
| E158Q | 1.33 | 0.27 | 20.20 | 1.44 | 0.80 | 55.37 | 37.78 | 40.82 | |
| E158S | 2.08 | 0.41 | 19.83 | 2.14 | 0.49 | 23.14 | 21.48 | 24.02 | |
| K159A | 0.70 | 0.41 | 58.70 | 0.79 | 0.60 | 76.82 | 67.76 | 40.82 | 231 |
| K159H | 0.48 | 0.36 | 75.44 | 0.67 | 0.45 | 66.99 | 71.22 | 40.82 | 208 |
| S261F | 0.23 | 0.22 | 96.52 | 0.22 | 0.23 | 106.85 | 101.69 | 35.71 | 179 |
| K159Q | 0.56 | 0.47 | 83.64 | 0.68 | 0.50 | 73.34 | 78.49 | 40.82 | 194 |
| K159S | 0.12 | 0.22 | 174.09 | 0.12 | 0.25 | 211.72 | 192.90 | 40.82 | 85 |
| A160R | 0.80 | 0.23 | 28.81 | 0.79 | 0.22 | 27.66 | 28.24 | 12.37 | |
| A160S | 0.96 | 0.35 | 36.05 | 0.92 | 0.35 | 38.60 | 37.32 | 12.37 | |
| A160V | 0.60 | 0.12 | 19.37 | 0.57 | 0.14 | 24.50 | 21.93 | 16.94 | |
| A160W | 0.54 | 0.16 | 29.20 | 0.46 | 0.18 | 38.26 | 33.73 | 24.02 | |
| A160Y | 0.14 | 0.13 | 93.33 | 0.15 | 0.14 | 90.82 | 92.08 | 12.37 | 377 |
| G161A | 0.70 | 0.45 | 63.69 | 0.65 | 0.40 | 61.94 | 62.82 | 12.37 | 323 |
| G161D | 0.60 | 0.35 | 58.33 | 0.62 | 0.36 | 57.97 | 58.15 | 16.94 | 213 |
| G161S | 1.15 | 0.52 | 45.65 | 1.20 | 0.53 | 44.31 | 44.98 | 28.73 | |
| K162L | 0.26 | 0.16 | 61.43 | 0.28 | 0.17 | 60.39 | 60.91 | 24.02 | 101 |
| K162M | 0.79 | 0.30 | 37.52 | 0.79 | 0.27 | 33.59 | 35.55 | 24.02 | |
| K162Y | 0.17 | 0.06 | 35.03 | 0.20 | 0.02 | 9.34 | 22.19 | 28.73 | |
| D163A | 0.69 | 0.29 | 42.22 | 1.01 | 0.35 | 34.97 | 38.59 | 36.75 | |
| D163E | 0.86 | 0.30 | 34.89 | 0.93 | 0.37 | 39.83 | 37.36 | 30.69 | |
| D163F | 0.40 | 0.17 | 43.14 | 0.44 | 0.16 | 36.15 | 39.64 | 29.57 | |
| D163K | 0.24 | 0.21 | 89.35 | 0.83 | 0.32 | 38.64 | 64.00 | 26.52 | 349 |
| D163L | 0.45 | 0.16 | 34.44 | 0.56 | 0.18 | 31.23 | 32.83 | 36.75 | |
| D163Q | 1.99 | 0.55 | 27.72 | 2.14 | 0.66 | 30.56 | 29.14 | 30.69 | |
| D163R | 0.30 | 0.32 | 107.31 | 0.25 | 0.14 | 56.94 | 82.13 | 30.69 | 180 |
| D163S | 3.27 | 2.06 | 63.08 | 4.19 | 2.74 | 65.33 | 64.20 | 32.76 | 284 |
| F164L | 0.21 | 0.05 | 23.79 | 0.19 | 0.07 | 35.06 | 29.43 | 4.69 | |
| F164M | 0.72 | 0.23 | 31.60 | 0.81 | 0.25 | 30.51 | 31.06 | 28.73 | |
| F164W | 0.20 | 0.07 | 37.85 | 0.13 | 0.05 | 40.54 | 39.20 | 28.73 | |
| V058R | 2.39 | 1.76 | 73.42 | 2.46 | 1.65 | 67.00 | 70.21 | 26.81 | 220 |
| V166A | 0.58 | 0.19 | 32.53 | 0.55 | 0.16 | 28.51 | 30.52 | 17.50 | |
| V166C | 0.28 | 0.04 | 14.44 | 0.24 | 0.04 | 16.05 | 15.24 | 28.73 | |
| V166E | 0.26 | 0.02 | 8.93 | 0.24 | 0.04 | 15.05 | 11.99 | 12.92 | |
| V166F | 0.38 | 0.16 | 40.83 | 0.53 | 0.20 | 37.61 | 39.22 | 28.50 | |
| V166G | 0.26 | 0.02 | 8.93 | 0.29 | 0.05 | 15.48 | 12.20 | 12.92 | |
| V166H | 0.16 | 0.03 | 19.75 | 0.18 | 0.04 | 23.22 | 21.48 | 12.92 | |
| V166L | 0.33 | 0.15 | 45.04 | 0.42 | 0.20 | 46.65 | 45.84 | 36.75 | |
| V166L | 1.25 | 0.29 | 23.37 | 1.33 | 0.47 | 35.72 | 29.55 | 28.50 | |
| V166Q | 0.80 | 0.30 | 38.05 | 0.92 | 0.26 | 28.05 | 33.05 | 28.50 | |
| V166R | 1.55 | 0.54 | 34.92 | 1.23 | 0.47 | 38.67 | 36.80 | 28.17 | |
| V166T | 0.39 | 0.14 | 36.19 | 0.36 | 0.15 | 41.71 | 38.95 | 14.03 | |
| V166W | 0.29 | 0.08 | 26.99 | 0.26 | 0.10 | 39.77 | 33.38 | 14.03 | |
| V166Y | 0.56 | 0.14 | 25.07 | 0.63 | 0.16 | 24.80 | 24.93 | 28.50 | |
| E167A | 0.32 | 0.11 | 32.97 | 0.27 | 0.11 | 41.88 | 37.43 | 14.03 | |
| E167D | 0.59 | 0.22 | 36.16 | 0.75 | 0.17 | 22.51 | 29.34 | 20.63 | |
| E167G | 0.39 | 0.14 | 37.00 | 0.46 | 0.17 | 36.78 | 36.89 | 28.50 | |
| E167K | 0.71 | 0.15 | 20.82 | 0.25 | 0.09 | 36.60 | 28.71 | 14.03 | |
| E167R | 0.58 | 0.14 | 24.76 | 0.71 | 0.18 | 25.28 | 25.02 | 28.50 | |
| E167S | 0.30 | 0.05 | 14.93 | 0.23 | 0.10 | 42.86 | 28.89 | 19.52 | |
| E167T | 0.55 | 0.16 | 30.16 | 0.58 | 0.16 | 27.45 | 28.80 | 20.67 | |
| E167Y | 0.33 | 0.09 | 25.68 | 0.35 | 0.07 | 20.54 | 23.11 | 16.69 | |
| I169L | 2.97 | 0.91 | 30.74 | 2.74 | 0.56 | 20.40 | 25.57 | 16.69 | |
| I169V | 0.33 | 0.09 | 26.63 | 0.33 | 0.14 | 40.66 | 33.64 | 19.52 | |
| K170Q | 0.62 | 0.19 | 30.75 | 0.59 | 0.16 | 26.71 | 28.73 | 20.67 | |
| K170R | 0.52 | 0.28 | 53.86 | 0.46 | 0.23 | 50.16 | 52.01 | 3.94 | 219 |
| L171V | 1.87 | 0.42 | 22.28 | 1.18 | 0.32 | 27.20 | 24.74 | 3.94 | |
| G172A | 2.97 | 0.78 | 26.37 | 2.61 | 0.46 | 17.81 | 22.09 | 3.94 | |
| G172C | 1.05 | 0.27 | 25.94 | 0.80 | 0.25 | 30.99 | 28.46 | 3.94 | |
| K173R | 1.86 | 0.62 | 33.14 | 1.59 | 0.44 | 27.44 | 30.29 | 3.94 | |
| L174A | 4.18 | 1.64 | 39.29 | 3.25 | 0.96 | 29.61 | 34.45 | 3.94 | |
| L174G | 3.79 | 1.29 | 34.07 | 2.32 | 0.74 | 31.82 | 32.95 | 3.94 | |
| L174H | 1.10 | 0.47 | 42.81 | 1.04 | 0.38 | 36.95 | 39.88 | 20.63 | |
| L174K | 10.56 | 5.96 | 56.41 | 6.89 | 2.82 | 40.94 | 48.68 | 3.94 | |
| L174M | 2.96 | 1.06 | 35.67 | 4.41 | 1.74 | 39.48 | 37.57 | 5.72 | |
| L174N | 1.95 | 0.65 | 33.28 | 1.71 | 0.45 | 26.08 | 29.68 | 5.72 | |
| L174Q | 2.66 | 0.85 | 32.03 | 2.72 | 0.61 | 22.47 | 27.25 | 5.72 | |
| L174R | 0.78 | 0.32 | 40.88 | 0.72 | 0.27 | 37.08 | 38.98 | 5.72 | |
| L174S | 0.82 | 0.29 | 35.79 | 0.58 | 0.21 | 36.16 | 35.97 | 5.72 | |
| L174T | 2.31 | 0.73 | 31.42 | 2.09 | 0.52 | 24.87 | 28.15 | 5.72 | |
| L174V | 1.67 | 0.45 | 26.80 | 1.60 | 0.40 | 25.16 | 25.98 | 5.72 | |
| K159N | 0.24 | 0.30 | 123.57 | 0.28 | 0.30 | 105.45 | 114.51 | 40.82 | 80 |
| L175T | 1.01 | 0.19 | 18.62 | 0.77 | 0.13 | 17.54 | 18.08 | 1.38 | |
| L175V | 0.80 | 0.11 | 14.33 | 0.87 | 0.11 | 12.67 | 13.50 | 1.38 | |
| L175Y | 0.61 | 0.11 | 17.92 | 0.62 | 0.06 | 9.25 | 13.59 | 1.38 | |
| R176L | 0.58 | 0.12 | 20.07 | 0.65 | 0.12 | 18.64 | 19.36 | 1.38 | |
| N178K | 0.19 | 0.05 | 28.35 | 0.15 | 0.07 | 48.82 | 38.58 | 1.38 | |
| N178M | 0.30 | 0.09 | 29.49 | 0.26 | 0.05 | 18.71 | 24.10 | 1.38 | |
| H179K | 2.92 | 0.96 | 32.68 | 3.49 | 1.02 | 29.13 | 30.90 | 16.98 | |
| H179M | 1.06 | 0.22 | 21.09 | 1.07 | 0.20 | 18.22 | 19.66 | 16.98 | |
| H179R | 1.44 | 0.52 | 36.11 | 1.59 | 0.48 | 30.36 | 33.23 | 16.98 | |
| H179S | 0.49 | 0.20 | 40.47 | 0.45 | 0.15 | 32.88 | 36.68 | 16.98 | |
| L180F | 1.04 | 0.27 | 25.41 | 1.11 | 0.24 | 22.04 | 23.72 | 16.98 | |
| L180I | 0.71 | 0.17 | 23.80 | 0.69 | 0.18 | 25.56 | 24.68 | 16.98 | |
| L180K | 0.39 | 0.14 | 35.29 | 0.57 | 0.00 | 0.00 | 17.65 | 36.75 | |
| Y183L | 0.61 | 0.44 | 71.77 | 1.29 | 0.36 | 28.09 | 49.93 | 1.44 | |
| F186Y | 1.02 | 0.27 | 26.07 | 1.12 | 0.21 | 18.55 | 22.31 | 1.44 | |
| H192S | 0.31 | 0.06 | 19.33 | 0.27 | 0.07 | 26.47 | 22.90 | 35.00 | |
| H193F | 0.30 | 0.13 | 42.24 | 0.33 | 0.14 | 41.41 | 41.83 | 27.54 | |
| H193Q | 0.20 | 0.09 | 46.67 | 0.26 | 0.00 | 0.00 | 23.33 | 0.00 | |
| H193R | 0.11 | 0.00 | 0.00 | 0.19 | 0.00 | 0.00 | 0.00 | 0.00 | |
| K195A | 0.26 | 0.04 | 16.70 | 0.37 | 0.00 | 0.00 | 8.35 | 0.00 | |
| K195H | 0.91 | 0.56 | 61.51 | 0.72 | 0.45 | 62.10 | 61.81 | 34.96 | 353 |
| K195L | 0.53 | 0.42 | 80.78 | 0.45 | 0.24 | 53.98 | 67.38 | 34.96 | 232 |
| K195N | 0.68 | 0.36 | 53.69 | 0.50 | 0.30 | 59.82 | 56.75 | 34.96 | 223 |
| K195R | 1.05 | 0.42 | 40.04 | 1.50 | 0.29 | 18.96 | 29.50 | 34.96 | |
| K195S | 0.22 | 0.00 | 0.00 | 0.24 | 0.00 | 0.00 | 0.00 | 0.00 | |
| K195V | 0.27 | 0.00 | 0.00 | 0.18 | 0.00 | 0.00 | 0.00 | 0.00 | |
| K195W | 0.22 | 0.08 | 36.04 | 0.21 | 0.06 | 30.33 | 33.18 | 35.00 | |
| K196L | 0.70 | 0.28 | 39.70 | 0.66 | 0.20 | 30.19 | 34.95 | 34.96 | |
| K196N | 0.24 | 0.00 | 0.00 | 0.23 | 0.00 | 0.00 | 0.00 | 0.00 | |
| K196R | 0.60 | 0.04 | 5.83 | 0.62 | 0.02 | 2.66 | 4.24 | 0.00 | |
| K196S | 0.37 | 0.04 | 10.56 | 0.36 | 0.08 | 23.18 | 16.87 | 0.00 | |
| K196T | 0.29 | 0.16 | 56.32 | 0.30 | 0.22 | 71.19 | 63.75 | 27.54 | 375 |
| K196W | 0.56 | 0.14 | 25.70 | 0.29 | 0.11 | 37.98 | 31.84 | 34.96 | |
| K196Y | 0.40 | 0.16 | 39.90 | 0.29 | 0.11 | 38.41 | 39.15 | 34.96 | |
| P197A | 0.48 | 0.00 | 0.00 | 0.44 | 0.00 | 0.00 | 0.00 | 0.00 | |
| P197D | 0.16 | 0.00 | 0.00 | 0.16 | 0.00 | 0.00 | 0.00 | 0.00 | |
| P197E | 0.28 | 0.00 | 0.00 | 0.23 | 0.00 | 0.00 | 0.00 | 0.00 | |
| P197F | 0.52 | 0.36 | 67.97 | 0.59 | 0.42 | 72.00 | 69.99 | 35.71 | 222 |
| P197G | 0.36 | 0.00 | 0.00 | 0.31 | 0.00 | 0.00 | 0.00 | 0.00 | |
| P197H | 0.69 | 0.00 | 0.00 | 0.73 | 0.00 | 0.00 | 0.00 | 0.00 | |
| P197K | 0.83 | 0.31 | 37.05 | 0.81 | 0.31 | 38.30 | 37.67 | 24.02 | |
| P197L | 0.93 | 0.00 | 0.00 | 0.62 | 0.00 | 0.00 | 0.00 | 0.00 | |
| P197R | 0.72 | 0.27 | 37.34 | 0.93 | 0.32 | 34.40 | 35.87 | 24.02 | |
| P197S | 0.62 | 0.00 | 0.00 | 0.92 | 0.00 | 0.00 | 0.00 | 0.00 | |
| G198A | 0.61 | 0.00 | 0.00 | 0.44 | 0.00 | 0.00 | 0.00 | 0.00 | |
| G198D | 0.59 | 0.00 | 0.00 | 0.56 | 0.00 | 0.00 | 0.00 | 0.00 | |
| G198H | 1.18 | 0.00 | 0.00 | 1.14 | 0.00 | 0.00 | 0.00 | 0.00 | |
| G198L | 0.49 | 0.40 | 81.66 | 0.46 | 0.43 | 93.58 | 87.62 | 35.71 | 162 |
| G198N | 1.55 | 0.21 | 13.26 | 1.36 | 0.17 | 12.79 | 13.02 | 0.00 | |
| G198Q | 0.98 | 0.00 | 0.00 | 1.05 | 0.08 | 7.18 | 3.59 | 0.00 | |
| G198R | 1.19 | 0.00 | 0.00 | 1.36 | 0.07 | 5.15 | 2.58 | 0.00 | |
| G198S | 0.41 | 0.07 | 17.94 | 0.52 | 0.07 | 14.23 | 16.08 | 0.00 | |
| G198W | 0.27 | 0.07 | 26.09 | 0.29 | 0.07 | 22.35 | 24.22 | 35.00 | |
| G198Y | 0.06 | 0.00 | 0.00 | 0.13 | 0.00 | 0.00 | 0.00 | 0.00 | |
| S202M | 0.37 | 0.21 | 55.17 | 0.30 | 0.14 | 46.37 | 50.77 | 24.67 | 227 |
| A412S | 0.20 | 0.20 | 97.26 | 0.23 | 0.15 | 66.96 | 82.11 | 28.04 | 245 |
| D030P | 0.41 | 0.18 | 43.15 | 0.38 | 0.17 | 45.28 | 44.21 | 30.73 | 386 |
| N205D | 0.85 | 0.18 | 21.22 | 1.27 | 0.49 | 38.68 | 29.95 | 0.00 | |
| N205E | 1.94 | 1.26 | 64.97 | 1.09 | 0.66 | 60.29 | 62.63 | 27.08 | 330 |
| N205G | 1.46 | 0.65 | 44.60 | 0.97 | 0.39 | 39.78 | 42.19 | 27.08 | |
| N205K | 0.73 | 0.45 | 61.35 | 0.77 | 0.45 | 58.87 | 60.11 | 35.71 | 379 |
| N205L | 0.98 | 0.74 | 75.71 | 1.83 | 2.65 | 144.54 | 110.13 | 31.65 | 98 |
| N205P | 1.37 | 0.91 | 66.52 | 1.28 | 0.67 | 52.43 | 59.48 | 27.08 | 233 |
| N205S | 1.45 | 0.80 | 55.05 | 0.96 | 0.46 | 48.12 | 51.58 | 27.08 | 234 |
| N205T | 0.29 | 0.28 | 95.18 | 0.32 | 0.26 | 79.32 | 87.25 | 27.08 | 209 |
| L037K | 0.35 | 0.27 | 78.61 | 0.53 | 0.29 | 53.62 | 66.12 | 36.50 | 320 |
| V206S | 0.45 | 0.10 | 22.04 | 0.45 | 0.12 | 26.36 | 24.20 | 35.00 | |
| I208K | 0.71 | 0.13 | 18.14 | 0.75 | 0.16 | 21.49 | 19.81 | 35.00 | |
| I208L | 0.91 | 0.53 | 58.27 | 0.77 | 0.46 | 60.57 | 59.42 | 27.08 | 237 |
| I208Q | 0.37 | 0.35 | 94.65 | 0.63 | 0.31 | 49.76 | 72.20 | 27.08 | 210 |
| I208R | 1.81 | 0.72 | 40.10 | 3.00 | 8.44 | 281.43 | 160.77 | 31.65 | 81 |
| I208S | 1.33 | 0.04 | 2.67 | 1.33 | 0.00 | 0.00 | 1.34 | 0.00 | |
| I208T | 1.46 | 0.12 | 8.11 | 1.51 | 0.01 | 0.50 | 4.30 | 0.00 | |
| I208V | 1.15 | 0.20 | 17.37 | 1.16 | 0.02 | 1.93 | 9.65 | 0.00 | |
| I208M | 0.43 | 0.21 | 48.65 | 0.29 | 0.14 | 49.23 | 48.94 | 28.17 | |
| K209L | 0.66 | 0.09 | 13.19 | 0.66 | 0.00 | 0.00 | 6.60 | 0.00 | |
| K209N | 0.90 | 0.09 | 9.59 | 0.87 | 0.00 | 0.00 | 4.79 | 0.00 | |
| K209R | 1.07 | 0.15 | 13.63 | 0.92 | 0.02 | 2.22 | 7.93 | 0.00 | |
| D212N | 1.85 | 0.35 | 18.79 | 1.80 | 0.34 | 19.07 | 18.93 | 1.44 | |
| D212S | 1.30 | 0.15 | 11.72 | 1.50 | 0.16 | 10.93 | 11.32 | 1.44 | |
| D212T | 1.45 | 0.18 | 12.36 | 1.25 | 0.15 | 11.84 | 12.10 | 1.44 | |
| D213A | 1.00 | 0.29 | 29.52 | 1.09 | 0.29 | 26.24 | 27.88 | 27.78 | |
| D213E | 0.73 | 0.46 | 63.17 | 0.73 | 0.47 | 64.69 | 63.93 | 35.71 | 287 |
| D213G | 1.66 | 0.33 | 19.69 | 1.71 | 0.18 | 10.65 | 15.17 | 14.25 | |
| D213H | 1.75 | 0.27 | 15.70 | 1.54 | 0.15 | 9.92 | 12.81 | 14.25 | |
| D213K | 1.50 | 0.40 | 26.72 | 1.21 | 0.29 | 23.95 | 25.33 | 14.25 | |
| D213L | 0.54 | 0.18 | 33.18 | 0.55 | 0.16 | 29.55 | 31.37 | 14.25 | |
| D213M | 0.83 | 0.26 | 30.94 | 0.82 | 0.20 | 24.50 | 27.72 | 14.25 | |
| D213N | 1.73 | 1.35 | 78.37 | 2.68 | 2.33 | 86.98 | 82.68 | 31.65 | 186 |
| D213Q | 1.40 | 0.49 | 35.17 | 1.37 | 0.34 | 25.12 | 30.14 | 14.25 | |
| D213R | 0.70 | 0.35 | 49.36 | 0.60 | 0.26 | 43.50 | 46.43 | 14.25 | |
| S215A | 2.07 | 1.04 | 50.36 | 2.17 | 1.29 | 59.27 | 54.82 | 36.49 | 242 |
| S215D | 1.12 | 0.49 | 43.59 | 1.06 | 0.62 | 58.22 | 50.90 | 36.49 | 243 |
| S215E | 0.48 | 0.49 | 101.77 | 0.63 | 0.49 | 78.14 | 89.96 | 36.49 | 148 |
| S215G | 1.90 | 0.80 | 41.92 | 1.86 | 0.82 | 43.75 | 42.83 | 36.49 | |
| S215H | 0.89 | 0.48 | 53.84 | 0.94 | 0.64 | 68.70 | 61.27 | 36.49 | 369 |
| S215K | 1.43 | 0.66 | 46.48 | 1.72 | 0.82 | 47.79 | 47.14 | 36.49 | |
| S215L | 1.08 | 0.48 | 44.92 | 1.05 | 0.57 | 54.41 | 49.67 | 36.49 | |
| S215Q | 1.30 | 0.69 | 53.17 | 1.05 | 0.33 | 30.91 | 42.04 | 19.93 | |
| S215R | 0.69 | 0.29 | 42.85 | 0.97 | 0.26 | 26.97 | 34.91 | 19.93 | |
| V277A | 0.24 | 0.42 | 171.66 | 0.26 | 0.49 | 189.90 | 180.78 | 37.53 | 129 |
| S215V | 1.17 | 0.57 | 48.67 | 1.27 | 0.66 | 51.78 | 50.22 | 19.93 | 247 |
| S215W | 0.64 | 0.40 | 62.97 | 0.82 | 0.44 | 53.38 | 58.17 | 19.93 | 248 |
| N219A | 0.54 | 0.38 | 69.25 | 0.84 | 0.35 | 41.54 | 55.40 | 19.93 | 249 |
| N219C | 0.18 | 0.08 | 41.69 | 0.15 | 0.06 | 39.93 | 40.81 | 27.78 | |
| N219E | 0.86 | 0.36 | 41.47 | 1.10 | 0.36 | 33.23 | 37.35 | 19.93 | |
| N219H | 0.63 | 0.16 | 25.66 | 0.54 | 0.04 | 7.84 | 16.75 | 19.93 | |
| N219I | 0.58 | 0.18 | 30.54 | 1.61 | 0.95 | 58.81 | 44.68 | 8.29 | |
| N219K | 1.05 | 0.51 | 48.85 | 2.10 | 1.06 | 50.35 | 49.60 | 8.29 | |
| N219L | 1.03 | 0.28 | 26.78 | 1.41 | 0.43 | 30.25 | 28.51 | 8.29 | |
| N219M | 0.73 | 0.16 | 22.13 | 1.12 | 0.25 | 22.02 | 22.08 | 8.29 | |
| N219R | 0.83 | 0.37 | 44.51 | 1.50 | 0.83 | 55.63 | 50.07 | 8.29 | 250 |
| N219S | 0.56 | 0.31 | 54.85 | 1.30 | 0.64 | 49.33 | 52.09 | 8.29 | 251 |
| N219T | 0.75 | 0.26 | 35.37 | 1.96 | 1.48 | 75.29 | 55.33 | 8.29 | 252 |
| N219W | 0.93 | 0.00 | 0.00 | 0.69 | 0.00 | 0.00 | 0.00 | 6.84 | |
| E220A | 0.30 | 0.06 | 20.27 | 0.20 | 0.00 | 0.00 | 10.13 | 6.84 | |
| E220H | 3.03 | 0.82 | 27.16 | 2.37 | 0.42 | 17.61 | 22.39 | 6.84 | |
| E220I | 1.70 | 0.42 | 24.73 | 1.30 | 0.16 | 12.40 | 18.56 | 6.84 | |
| E220M | 1.25 | 0.37 | 29.58 | 1.15 | 0.20 | 17.19 | 23.39 | 6.84 | |
| E220S | 2.30 | 0.69 | 29.85 | 1.59 | 0.31 | 19.20 | 24.53 | 6.84 | |
| E220T | 3.37 | 0.87 | 25.87 | 2.36 | 0.42 | 17.85 | 21.86 | 6.84 | |
| E220V | 4.24 | 2.32 | 54.75 | 2.95 | 1.09 | 36.86 | 45.80 | 6.84 | 384 |
| S221A | 0.45 | 0.10 | 21.61 | 0.49 | 0.11 | 21.56 | 21.59 | 27.78 | |
| S221C | 0.57 | 0.06 | 10.71 | 0.65 | 0.08 | 12.94 | 11.82 | 27.78 | |
| S221M | 0.31 | 0.04 | 14.17 | 0.28 | 0.05 | 16.43 | 15.30 | 27.78 | |
| S221T | 0.81 | 0.17 | 21.29 | 0.97 | 0.21 | 21.44 | 21.36 | 27.78 | |
| S221V | 0.19 | 0.10 | 51.32 | 0.14 | 0.06 | 41.96 | 46.64 | 27.78 | |
| T222K | 0.90 | 0.28 | 30.87 | 1.48 | 0.66 | 44.68 | 37.77 | 25.54 | |
| T222N | 0.24 | 0.20 | 82.55 | 0.43 | 0.18 | 40.77 | 61.66 | 32.47 | 361 |
| L230I | 0.71 | 0.37 | 52.91 | 0.68 | 0.29 | 42.46 | 47.68 | 33.75 | |
| T232F | 0.32 | 0.14 | 44.22 | 0.41 | 0.18 | 42.60 | 43.41 | 25.54 | |
| T232S | 1.43 | 0.46 | 32.20 | 1.11 | 0.41 | 36.90 | 34.55 | 7.79 | |
| Q233K | 1.38 | 0.34 | 24.64 | 1.45 | 0.33 | 23.05 | 23.84 | 7.79 | |
| Q233L | 1.10 | 0.22 | 19.94 | 1.03 | 0.18 | 17.28 | 18.61 | 7.79 | |
| Q233R | 0.76 | 0.33 | 43.34 | 0.75 | 0.26 | 34.98 | 39.16 | 32.47 | |
| Q234M | 1.08 | 0.65 | 59.85 | 1.18 | 0.58 | 49.11 | 54.48 | 7.79 | 254 |
| S235A | 0.25 | 0.10 | 38.98 | 0.28 | 0.10 | 37.45 | 38.22 | 25.54 | |
| S235G | 0.82 | 0.21 | 25.52 | 0.81 | 0.17 | 21.14 | 23.33 | 7.79 | |
| S235K | 0.49 | 0.10 | 19.53 | 0.40 | 0.02 | 5.99 | 12.76 | 7.79 | |
| N205A | 0.39 | 0.40 | 103.37 | 0.37 | 0.40 | 110.82 | 107.10 | 35.71 | 173 |
| P236A | 1.65 | 0.09 | 5.60 | 1.51 | 0.05 | 3.59 | 4.59 | 0.00 | |
| P236G | 1.29 | 0.04 | 2.75 | 1.20 | 0.00 | 0.00 | 1.37 | 0.00 | |
| P236H | 0.57 | 0.00 | 0.00 | 0.47 | 0.00 | 0.00 | 0.00 | 0.00 | |
| P236K | 0.49 | 0.00 | 0.00 | 0.44 | 0.00 | 0.00 | 0.00 | 0.00 | |
| P236R | 0.56 | 0.00 | 0.00 | 0.47 | 0.00 | 0.00 | 0.00 | 0.00 | |
| P236S | 0.34 | 0.14 | 41.41 | 0.55 | 0.17 | 31.25 | 36.33 | 25.54 | |
| V237A | 1.28 | 0.43 | 33.36 | 1.30 | 0.73 | 56.01 | 44.68 | 24.67 | |
| V237E | 0.47 | 0.00 | 0.00 | 0.45 | 0.00 | 0.00 | 0.00 | 0.00 | |
| V237H | 0.31 | 0.13 | 42.20 | 0.44 | 0.16 | 37.57 | 39.88 | 25.54 | |
| V237L | 1.55 | 0.07 | 4.71 | 1.66 | 0.02 | 1.17 | 2.94 | 0.00 | |
| V237N | 1.37 | 0.00 | 0.33 | 1.24 | 0.00 | 0.00 | 0.16 | 0.00 | |
| V237Q | 2.10 | 1.66 | 79.06 | 3.44 | 2.48 | 72.08 | 75.57 | 31.65 | 197 |
| V237R | 0.72 | 0.00 | 0.00 | 0.78 | 0.00 | 0.00 | 0.00 | 0.00 | |
| V237S | 0.67 | 0.00 | 0.00 | 0.70 | 0.00 | 0.00 | 0.00 | 0.00 | |
| V237T | 0.25 | 0.13 | 51.49 | 0.37 | 0.15 | 40.70 | 46.10 | 25.54 | |
| V237W | 0.49 | 0.00 | 0.00 | 0.53 | 0.00 | 0.00 | 0.00 | 0.00 | |
| A238D | 1.65 | 0.02 | 1.21 | 1.31 | 0.00 | 0.00 | 0.61 | 0.00 | |
| A238E | 0.81 | 0.00 | 0.00 | 0.74 | 0.00 | 0.00 | 0.00 | 0.00 | |
| A238H | 1.07 | 0.00 | 0.00 | 0.99 | 0.00 | 0.00 | 0.00 | 0.00 | |
| A238R | 0.35 | 0.17 | 47.94 | 0.34 | 0.15 | 45.07 | 46.50 | 24.67 | |
| A238S | 0.77 | 0.00 | 0.00 | 0.90 | 0.00 | 0.00 | 0.00 | 0.00 | |
| A238T | 0.68 | 0.00 | 0.00 | 0.82 | 0.00 | 0.00 | 0.00 | 0.00 | |
| A239K | 0.29 | 0.10 | 33.85 | 0.35 | 0.15 | 41.13 | 37.49 | 25.54 | |
| T240A | 0.53 | 0.00 | 0.00 | 0.90 | 0.00 | 0.00 | 0.00 | 0.00 | |
| T240K | 2.13 | 0.73 | 34.34 | 1.39 | 0.53 | 37.95 | 36.15 | 24.67 | |
| T240M | 0.84 | 0.00 | 0.00 | 0.92 | 0.00 | 0.00 | 0.00 | 0.00 | |
| T240Q | 1.21 | 1.38 | 114.10 | 1.21 | 0.57 | 46.78 | 80.44 | 27.49 | 190 |
| T240R | 1.33 | 0.04 | 3.31 | 1.73 | 0.04 | 2.22 | 2.76 | 0.00 | |
| T240S | 0.39 | 0.00 | 0.00 | 0.44 | 0.00 | 0.00 | 0.00 | 0.00 | |
| T240V | 2.25 | 0.22 | 9.76 | 2.18 | 0.16 | 7.54 | 8.65 | 0.00 | |
| Y242F | 0.52 | 0.09 | 17.08 | 0.37 | 0.11 | 30.39 | 23.73 | 27.87 | |
| N245H | 1.04 | 0.30 | 28.83 | 0.75 | 0.17 | 22.00 | 25.42 | 27.87 | |
| V247I | 0.18 | 0.12 | 63.29 | 0.14 | 0.09 | 59.58 | 61.43 | 27.87 | 265 |
| V247L | 1.89 | 0.48 | 25.41 | 1.52 | 0.35 | 23.06 | 24.24 | 27.87 | |
| I251L | 0.59 | 0.48 | 81.48 | 0.89 | 0.79 | 89.42 | 85.45 | 31.65 | 172 |
| I251M | 0.44 | 0.35 | 78.23 | 0.27 | 0.14 | 53.02 | 65.63 | 27.49 | 262 |
| V253I | 0.88 | 0.13 | 14.74 | 0.88 | 0.15 | 17.11 | 15.92 | 37.91 | |
| K255Q | 2.57 | 0.88 | 34.03 | 1.83 | 0.56 | 30.70 | 32.36 | 27.87 | |
| K255R | 0.18 | 0.13 | 72.47 | 0.15 | 0.07 | 47.51 | 59.99 | 27.87 | 261 |
| P257K | 0.36 | 0.03 | 7.84 | 0.26 | 0.06 | 21.69 | 14.76 | 39.27 | |
| P257L | 0.50 | 0.09 | 18.95 | 0.38 | 0.09 | 24.57 | 21.76 | 39.27 | |
| P257N | 0.51 | 0.14 | 27.23 | 0.46 | 0.12 | 25.54 | 26.38 | 39.27 | |
| P257R | 1.29 | 0.34 | 26.64 | 0.97 | 0.25 | 26.30 | 26.47 | 39.27 | |
| P257V | 0.63 | 0.25 | 39.24 | 0.46 | 0.20 | 43.44 | 41.34 | 39.27 | |
| D258N | 0.88 | 0.46 | 52.76 | 0.95 | 0.38 | 39.96 | 46.36 | 39.27 | |
| A259E | 0.57 | 0.28 | 48.25 | 0.55 | 0.23 | 42.21 | 45.23 | 39.27 | |
| A259G | 0.69 | 0.28 | 40.49 | 1.65 | 0.73 | 44.17 | 42.33 | 16.82 | |
| A259K | 0.12 | 0.16 | 126.02 | 0.31 | 0.23 | 73.89 | 99.95 | 16.82 | 127 |
| A259P | 2.24 | 0.99 | 44.47 | 4.05 | 3.23 | 79.65 | 62.06 | 16.82 | 344 |
| A259Q | 0.83 | 0.39 | 47.26 | 0.46 | 0.18 | 39.34 | 43.30 | 35.77 | |
| A259S | 1.89 | 0.93 | 49.21 | 1.64 | 0.69 | 42.35 | 45.78 | 40.52 | |
| A259T | 0.15 | 0.07 | 46.23 | 0.35 | 0.08 | 24.14 | 35.18 | 40.52 | |
| A259V | 0.24 | 0.11 | 47.58 | 0.46 | 0.14 | 29.72 | 38.65 | 40.52 | |
| A259W | 0.45 | 0.21 | 47.39 | 0.95 | 0.25 | 26.17 | 36.78 | 40.52 | |
| A259Y | 1.00 | 0.34 | 33.40 | 0.96 | 0.04 | 3.86 | 18.63 | 40.52 | |
| K260A | 0.86 | 0.35 | 41.05 | 0.27 | 0.09 | 31.31 | 36.18 | 40.52 | |
| K260G | 0.14 | 0.10 | 69.60 | 0.21 | 0.07 | 33.41 | 51.50 | 40.52 | 263 |
| K260H | 1.55 | 0.37 | 23.60 | 1.69 | 0.81 | 48.10 | 35.85 | 32.16 | |
| K260L | 0.56 | 0.34 | 61.43 | 2.27 | 0.71 | 31.13 | 46.28 | 40.52 | |
| K260M | 0.15 | 0.20 | 140.69 | 0.46 | 0.30 | 65.57 | 103.13 | 26.52 | 350 |
| K260Q | 0.65 | 0.35 | 53.52 | 0.64 | 0.23 | 36.63 | 45.08 | 37.83 | |
| K260R | 0.96 | 0.50 | 51.53 | 0.99 | 0.44 | 44.35 | 47.94 | 37.83 | |
| K260S | 1.43 | 0.48 | 33.22 | 1.53 | 0.61 | 40.14 | 36.68 | 37.83 | |
| L174W | 1.82 | 2.44 | 134.26 | 1.82 | 1.09 | 60.18 | 97.22 | 20.63 | 117 |
| G027H | 0.44 | 0.27 | 61.92 | 0.56 | 0.39 | 69.63 | 65.78 | 30.98 | 259 |
| S261K | 3.59 | 0.97 | 27.13 | 4.01 | 2.04 | 50.92 | 39.03 | 32.16 | |
| S261M | 1.27 | 0.00 | 0.00 | 0.84 | 0.00 | 0.00 | 0.00 | 27.90 | |
| S261N | 3.89 | 0.00 | 0.00 | 4.47 | 0.73 | 16.33 | 8.16 | 27.90 | |
| S261Q | 2.40 | 0.00 | 0.00 | 1.11 | 0.00 | 0.00 | 0.00 | 27.90 | |
| S261R | 3.86 | 0.00 | 0.00 | 3.28 | 0.00 | 0.00 | 0.00 | 27.90 | |
| S261T | 2.14 | 0.05 | 2.46 | 2.68 | 0.00 | 0.00 | 1.23 | 27.90 | |
| S261V | 2.53 | 0.40 | 15.85 | 1.62 | 0.08 | 4.86 | 10.35 | 27.90 | |
| S261W | 0.26 | 0.00 | 0.00 | 0.40 | 0.00 | 0.00 | 0.00 | 0.00 | |
| L263K | 3.32 | 0.00 | 0.00 | 3.36 | 0.00 | 0.00 | 0.00 | 0.00 | |
| L263M | 0.54 | 0.00 | 0.00 | 0.50 | 0.00 | 0.00 | 0.00 | 0.00 | |
| L263R | 1.65 | 0.00 | 0.00 | 1.98 | 0.00 | 0.00 | 0.00 | 0.00 | |
| L263T | 0.30 | 0.24 | 81.59 | 0.26 | 0.28 | 108.35 | 94.97 | 32.47 | 363 |
| L263V | 0.48 | 0.21 | 43.47 | 0.38 | 0.15 | 40.48 | 41.97 | 35.91 | |
| V265I | 0.22 | 0.14 | 63.70 | 0.25 | 0.13 | 52.11 | 57.90 | 32.29 | 268 |
| T269A | 0.21 | 0.09 | 42.51 | 0.19 | 0.07 | 38.30 | 40.40 | 35.91 | |
| T269D | 0.55 | 0.23 | 40.79 | 0.44 | 0.20 | 46.08 | 43.43 | 27.87 | |
| T269S | 1.40 | 0.59 | 41.79 | 1.03 | 0.33 | 32.14 | 36.96 | 35.91 | |
| I271L | 1.26 | 0.37 | 29.47 | 0.99 | 0.26 | 25.94 | 27.70 | 11.35 | |
| I271V | 0.62 | 0.47 | 77.15 | 0.54 | 1.65 | 308.12 | 192.64 | 32.26 | 77 |
| V272T | 0.60 | 0.25 | 42.01 | 0.60 | 0.26 | 42.77 | 42.39 | 32.26 | |
| F273H | 0.34 | 0.07 | 19.29 | 0.25 | 0.01 | 5.23 | 12.26 | 27.49 | |
| F273Y | 0.23 | 0.06 | 27.03 | 0.18 | 0.05 | 25.20 | 26.12 | 27.49 | |
| T274A | 0.51 | 0.18 | 34.78 | 0.51 | 0.19 | 37.08 | 35.93 | 37.65 | |
| Q276D | 0.58 | 0.30 | 51.03 | 0.55 | 0.27 | 48.05 | 49.54 | 28.26 | |
| Q276E | 0.22 | 0.16 | 69.20 | 0.25 | 0.19 | 75.40 | 72.30 | 37.23 | 226 |
| Q276H | 0.50 | 0.17 | 33.87 | 0.37 | 0.12 | 32.97 | 33.42 | 32.26 | |
| Q276R | 3.03 | 0.94 | 31.02 | 2.20 | 0.62 | 28.07 | 29.54 | 35.77 | |
| Q276S | 0.47 | 0.21 | 44.61 | 0.52 | 0.17 | 33.40 | 39.00 | 28.26 | |
| Q155D | 0.87 | 0.63 | 72.62 | 0.94 | 0.53 | 56.55 | 64.59 | 34.46 | 272 |
| L144G | 0.28 | 0.19 | 68.05 | 0.24 | 0.17 | 70.19 | 69.12 | 28.04 | 215 |
| V277E | 0.78 | 0.58 | 73.86 | 0.69 | 0.32 | 45.99 | 59.93 | 32.26 | 273 |
| V277H | 2.85 | 1.20 | 42.00 | 3.17 | 2.11 | 66.44 | 54.22 | 22.57 | 274 |
| V277K | 4.72 | 1.49 | 31.56 | 3.90 | 1.32 | 33.82 | 32.69 | 22.57 | |
| V277M | 0.56 | 0.21 | 36.67 | 0.48 | 0.17 | 34.86 | 35.77 | 22.57 | |
| V277N | 1.44 | 0.53 | 36.37 | 1.50 | 0.52 | 34.40 | 35.38 | 22.57 | |
| V277Q | 1.08 | 0.44 | 40.84 | 1.05 | 0.41 | 39.25 | 40.04 | 22.57 | |
| V277R | 0.20 | 0.09 | 45.91 | 0.18 | 0.08 | 43.36 | 44.63 | 22.57 | |
| V277S | 1.85 | 0.57 | 30.90 | 1.86 | 0.54 | 28.82 | 29.86 | 22.57 | |
| V277Y | 0.85 | 0.29 | 34.22 | 0.78 | 0.25 | 31.77 | 32.99 | 35.48 | |
| L278A | 0.67 | 0.36 | 54.36 | 0.78 | 0.31 | 39.25 | 46.80 | 37.65 | |
| L278E | 0.48 | 0.26 | 53.40 | 0.59 | 0.28 | 46.41 | 49.90 | 26.56 | |
| L278F | 1.01 | 0.41 | 40.07 | 0.98 | 0.36 | 36.35 | 38.21 | 26.56 | |
| L278G | 0.92 | 0.45 | 48.75 | 0.90 | 0.46 | 51.44 | 50.09 | 37.65 | 275 |
| L278H | 0.18 | 0.09 | 48.36 | 0.16 | 0.10 | 59.38 | 53.87 | 40.67 | 276 |
| L278K | 0.44 | 0.27 | 62.36 | 0.58 | 0.27 | 47.14 | 54.75 | 40.67 | 277 |
| L278N | 0.75 | 0.50 | 67.04 | 0.71 | 0.46 | 65.32 | 66.18 | 40.67 | 255 |
| L278S | 1.14 | 0.56 | 49.25 | 1.39 | 0.48 | 34.38 | 41.81 | 26.56 | |
| L278T | 1.70 | 0.67 | 39.30 | 1.62 | 0.59 | 36.51 | 37.91 | 35.48 | |
| L278Y | 0.49 | 0.18 | 36.80 | 0.38 | 0.15 | 40.29 | 38.55 | 40.67 | |
| K279A | 0.23 | 0.09 | 40.52 | 0.23 | 0.07 | 32.32 | 36.42 | 35.48 | |
| K279R | 0.88 | 0.32 | 36.44 | 0.90 | 0.27 | 30.57 | 33.50 | 26.56 | |
| S282D | 0.68 | 0.22 | 32.40 | 0.52 | 0.18 | 35.63 | 34.01 | 35.77 | |
| S282G | 2.84 | 2.20 | 77.52 | 2.30 | 0.83 | 36.14 | 56.83 | 40.67 | 279 |
| S282Q | 0.29 | 0.15 | 51.37 | 0.30 | 0.24 | 81.01 | 66.19 | 41.91 | 228 |
| Q283E | 0.85 | 0.38 | 44.84 | 1.09 | 0.34 | 31.24 | 38.04 | 41.91 | |
| N141Y | 0.21 | 0.25 | 121.79 | 0.22 | 0.22 | 100.00 | 110.90 | 36.07 | 164 |
| D284E | 1.09 | 0.20 | 18.42 | 1.06 | 0.17 | 15.59 | 17.01 | 14.90 | |
| D284Q | 0.41 | 0.57 | 138.22 | 0.46 | 0.49 | 106.00 | 122.11 | 37.53 | 96 |
| V277C | 0.24 | 0.38 | 161.89 | 0.22 | 0.41 | 185.26 | 173.58 | 37.53 | 130 |
| E285F | 0.47 | 0.21 | 44.68 | 0.46 | 0.19 | 40.90 | 42.79 | 16.94 | |
| E285H | 0.47 | 0.10 | 20.89 | 0.14 | 0.07 | 50.36 | 35.62 | 14.90 | |
| E285M | 0.29 | 0.20 | 67.13 | 0.36 | 0.22 | 61.33 | 64.23 | 24.30 | 156 |
| E285Q | 0.27 | 0.14 | 51.48 | 0.31 | 0.15 | 46.57 | 49.02 | 37.88 | |
| E285Y | 0.83 | 0.48 | 57.81 | 1.18 | 0.53 | 44.74 | 51.27 | 24.30 | 285 |
| Y288W | 0.41 | 0.07 | 17.73 | 0.58 | 0.15 | 26.36 | 22.05 | 29.23 | |
| F290I | 0.34 | 0.15 | 45.54 | 0.25 | 0.07 | 28.31 | 36.92 | 18.00 | |
| F290M | 0.41 | 0.21 | 51.41 | 0.37 | 0.19 | 51.44 | 51.42 | 18.12 | 286 |
| G291S | 4.95 | 1.80 | 36.27 | 3.69 | 1.66 | 44.96 | 40.62 | 23.75 | |
| G291V | 2.96 | 0.95 | 32.10 | 2.31 | 0.78 | 33.86 | 32.98 | 23.75 | |
| E292A | 3.34 | 1.44 | 43.12 | 2.97 | 1.34 | 45.12 | 44.12 | 23.75 | |
| E292C | 1.98 | 0.73 | 36.73 | 1.80 | 0.62 | 34.46 | 35.59 | 23.75 | |
| E292F | 1.14 | 0.48 | 41.65 | 1.45 | 0.52 | 36.03 | 38.84 | 23.75 | |
| E292G | 2.53 | 0.58 | 23.00 | 2.16 | 0.75 | 34.79 | 28.89 | 23.75 | |
| E292N | 2.34 | 1.02 | 43.39 | 2.08 | 0.72 | 34.74 | 39.06 | 23.75 | |
| E292P | 3.30 | 1.22 | 36.99 | 5.31 | 3.29 | 61.99 | 49.49 | 23.75 | |
| E292V | 1.53 | 0.98 | 64.31 | 2.05 | 2.11 | 103.15 | 83.73 | 36.18 | 184 |
| T293A | 1.45 | 0.53 | 36.42 | 1.21 | 0.40 | 32.91 | 34.66 | 40.64 | |
| I301A | 1.63 | 0.41 | 24.93 | 1.20 | 0.31 | 25.59 | 25.26 | 31.06 | |
| I301V | 2.14 | 0.67 | 31.52 | 1.92 | 0.48 | 25.07 | 28.29 | 31.06 | |
| G305D | 0.75 | 0.40 | 53.69 | 0.92 | 0.44 | 47.63 | 50.66 | 24.30 | 288 |
| G305E | 1.29 | 0.30 | 23.10 | 1.94 | 0.55 | 28.35 | 25.72 | 29.23 | |
| G305N | 0.23 | 0.20 | 86.27 | 0.31 | 0.18 | 55.89 | 71.08 | 24.30 | 157 |
| V102H | 0.27 | 0.17 | 61.76 | 0.45 | 0.33 | 73.70 | 67.73 | 24.78 | 316 |
| T306E | 0.71 | 0.29 | 41.27 | 0.88 | 0.46 | 51.76 | 46.51 | 32.76 | |
| T306S | 2.25 | 0.78 | 34.72 | 2.50 | 1.19 | 47.66 | 41.19 | 32.76 | |
| L307G | 0.79 | 0.36 | 46.11 | 0.69 | 0.26 | 38.07 | 42.09 | 35.77 | |
| L307N | 3.35 | 1.29 | 38.46 | 2.38 | 0.86 | 36.03 | 37.24 | 17.30 | |
| L307S | 1.48 | 0.57 | 38.21 | 1.37 | 0.50 | 36.28 | 37.25 | 35.77 | |
| L307T | 3.56 | 1.74 | 48.73 | 2.52 | 0.88 | 34.88 | 41.80 | 17.30 | |
| L307W | 1.62 | 0.46 | 28.40 | 0.71 | 0.25 | 34.48 | 31.44 | 17.30 | |
| S308G | 1.52 | 0.41 | 27.07 | 1.20 | 0.23 | 19.46 | 23.26 | 17.30 | |
| S308H | 0.41 | 0.06 | 15.52 | 0.27 | 0.07 | 24.35 | 19.93 | 21.52 | |
| S308K | 1.09 | 0.21 | 19.37 | 1.23 | 0.22 | 18.19 | 18.78 | 21.52 | |
| S308N | 1.49 | 0.38 | 25.68 | 1.66 | 0.41 | 24.57 | 25.13 | 21.52 | |
| S308R | 0.57 | 0.12 | 20.30 | 0.63 | 0.12 | 18.76 | 19.53 | 21.52 | |
| S308T | 1.25 | 0.41 | 32.91 | 1.35 | 0.54 | 39.96 | 36.43 | 32.12 | |
| I309E | 2.92 | 1.66 | 56.92 | 2.82 | 1.43 | 50.69 | 53.81 | 32.12 | 291 |
| I309G | 3.20 | 1.50 | 46.79 | 4.15 | 1.24 | 29.75 | 38.27 | 32.12 | |
| I309H | 0.43 | 0.23 | 53.50 | 0.37 | 0.21 | 56.15 | 54.82 | 32.12 | 292 |
| I309K | 2.23 | 0.50 | 22.50 | 2.06 | 0.45 | 22.04 | 22.27 | 32.12 | |
| I309L | 0.50 | 0.28 | 57.03 | 0.42 | 0.20 | 47.42 | 52.22 | 32.12 | 293 |
| I309M | 2.38 | 0.97 | 40.62 | 2.28 | 0.81 | 35.61 | 38.12 | 32.12 | |
| I309N | 5.77 | 1.54 | 26.60 | 6.49 | 1.75 | 27.00 | 26.80 | 29.86 | |
| I309Q | 1.54 | 0.71 | 46.49 | 1.37 | 0.52 | 38.06 | 42.27 | 35.48 | |
| I309S | 2.69 | 1.14 | 42.51 | 1.93 | 0.72 | 37.08 | 39.80 | 35.77 | |
| I309T | 2.81 | 0.77 | 27.43 | 2.86 | 0.62 | 21.80 | 24.62 | 29.86 | |
| I309V | 0.73 | 0.25 | 34.55 | 0.91 | 0.23 | 25.42 | 29.99 | 29.86 | |
| M310A | 5.23 | 2.88 | 55.00 | 3.97 | 1.52 | 38.24 | 46.62 | 18.00 | |
| M310F | 1.88 | 0.77 | 41.23 | 1.73 | 0.79 | 45.70 | 43.46 | 27.54 | |
| M310G | 0.80 | 0.26 | 32.63 | 0.64 | 0.21 | 32.71 | 32.67 | 18.12 | |
| M310Q | 0.41 | 0.30 | 73.59 | 0.38 | 0.14 | 37.72 | 55.65 | 18.00 | 294 |
| M310R | 0.45 | 0.34 | 74.70 | 0.58 | 0.39 | 66.55 | 70.62 | 27.54 | 216 |
| M310R | 0.33 | 0.24 | 72.45 | 0.32 | 0.23 | 72.44 | 72.45 | 26.52 | 216 |
| M310V | 0.45 | 0.12 | 26.15 | 0.34 | 0.04 | 12.20 | 19.18 | 18.12 | |
| D034W | 0.21 | 0.08 | 38.57 | 0.16 | 0.16 | 95.98 | 67.27 | 23.34 | 382 |
| R311H | 0.49 | 0.23 | 47.42 | 0.56 | 0.26 | 47.03 | 47.23 | 24.30 | |
| R311K | 0.43 | 0.28 | 65.85 | 0.27 | 0.11 | 39.44 | 52.64 | 48.62 | 298 |
| R311Q | 0.81 | 0.43 | 52.28 | 0.52 | 0.20 | 38.67 | 45.47 | 48.62 | |
| R311S | 0.36 | 0.21 | 57.54 | 0.25 | 0.10 | 39.80 | 48.67 | 48.62 | |
| S312K | 0.61 | 0.30 | 49.39 | 0.61 | 0.26 | 43.04 | 46.22 | 32.29 | |
| S312N | 1.80 | 0.76 | 42.25 | 2.62 | 0.80 | 30.63 | 36.44 | 32.29 | |
| S312T | 1.76 | 0.37 | 21.07 | 1.48 | 0.28 | 19.07 | 20.07 | 29.86 | |
| M313A | 0.53 | 0.17 | 32.60 | 0.51 | 0.14 | 27.09 | 29.85 | 29.86 | |
| M313E | 0.67 | 0.19 | 28.68 | 0.84 | 0.20 | 23.66 | 26.17 | 21.52 | |
| M313H | 0.71 | 0.19 | 27.03 | 0.64 | 0.14 | 21.49 | 24.26 | 21.52 | |
| M313K | 7.00 | 3.27 | 46.72 | 5.21 | 1.60 | 30.60 | 38.66 | 29.86 | |
| M313L | 0.98 | 0.26 | 26.98 | 1.34 | 0.60 | 44.47 | 35.72 | 26.64 | |
| M313P | 1.21 | 0.29 | 24.39 | 0.90 | 0.23 | 25.51 | 24.95 | 26.64 | |
| M313R | 0.74 | 0.18 | 24.88 | 0.63 | 0.11 | 17.13 | 21.00 | 26.64 | |
| M313S | 1.23 | 0.30 | 24.22 | 1.26 | 0.29 | 22.95 | 23.59 | 21.52 | |
| M313T | 1.79 | 0.50 | 28.07 | 2.20 | 0.46 | 20.93 | 24.50 | 21.52 | |
| M313V | 0.64 | 0.14 | 21.26 | 0.59 | 0.09 | 15.37 | 18.32 | 26.64 | |
| M313Y | 0.93 | 0.24 | 25.68 | 0.83 | 0.26 | 31.26 | 28.47 | 24.88 | |
| K314A | 0.71 | 0.22 | 30.86 | 0.75 | 0.17 | 22.41 | 26.63 | 24.88 | |
| K314D | 1.22 | 0.26 | 21.33 | 0.66 | 0.27 | 41.21 | 31.27 | 24.53 | |
| K314H | 0.82 | 0.19 | 22.69 | 0.88 | 0.23 | 26.57 | 24.63 | 24.88 | |
| K314Q | 1.62 | 0.33 | 20.41 | 1.42 | 0.63 | 44.38 | 32.40 | 32.16 | |
| K314R | 0.39 | 0.14 | 34.85 | 0.41 | 0.14 | 34.85 | 34.85 | 24.53 | |
| K314S | 1.98 | 0.74 | 37.48 | 1.32 | 0.36 | 27.45 | 32.47 | 35.77 | |
| S315A | 1.01 | 0.19 | 18.60 | 0.88 | 0.19 | 21.88 | 20.24 | 24.53 | |
| S315E | 0.23 | 0.08 | 33.41 | 0.19 | 0.05 | 26.74 | 30.07 | 24.53 | |
| S315H | 1.92 | 0.47 | 24.32 | 1.88 | 0.41 | 21.79 | 23.06 | 24.53 | |
| S315R | 1.29 | 0.39 | 29.93 | 1.03 | 0.36 | 35.28 | 32.60 | 24.53 | |
| F380L | 0.37 | 0.25 | 68.26 | 0.65 | 0.60 | 91.82 | 80.04 | 31.91 | 191 |
| L317A | 4.33 | 1.78 | 41.11 | 5.13 | 2.48 | 48.36 | 44.74 | 21.73 | |
| L317D | 0.20 | 0.10 | 47.06 | 0.44 | 0.15 | 33.71 | 40.39 | 21.73 | |
| L317H | 0.72 | 0.28 | 39.28 | 0.78 | 0.28 | 36.25 | 37.77 | 21.73 | |
| L317K | 2.81 | 1.09 | 38.73 | 3.91 | 1.29 | 32.91 | 35.82 | 21.73 | |
| L317N | 0.63 | 0.50 | 79.05 | 0.43 | 0.22 | 51.35 | 65.20 | 35.77 | 266 |
| L317Q | 4.95 | 1.42 | 28.62 | 4.37 | 1.55 | 35.42 | 32.02 | 35.35 | |
| L317R | 0.23 | 0.08 | 33.76 | 0.25 | 0.08 | 30.30 | 32.03 | 21.73 | |
| L317S | 2.67 | 0.96 | 35.90 | 2.48 | 0.82 | 32.90 | 34.40 | 35.35 | |
| L317T | 0.99 | 0.36 | 36.57 | 1.08 | 0.45 | 41.38 | 38.98 | 21.73 | |
| L318D | 0.76 | 0.29 | 37.62 | 0.68 | 0.18 | 26.05 | 31.83 | 40.64 | |
| L318I | 0.84 | 0.26 | 30.78 | 0.92 | 0.24 | 26.19 | 28.48 | 24.88 | |
| L318K | 0.72 | 0.48 | 66.76 | 0.68 | 0.32 | 46.84 | 56.80 | 35.42 | 301 |
| L318M | 0.37 | 0.23 | 61.58 | 0.36 | 0.20 | 56.16 | 58.87 | 35.42 | 302 |
| L318N | 0.86 | 0.49 | 57.45 | 0.70 | 0.38 | 54.96 | 56.21 | 35.42 | 303 |
| L318Q | 0.61 | 0.32 | 52.57 | 0.51 | 0.30 | 58.75 | 55.66 | 35.42 | 304 |
| L318S | 0.42 | 0.17 | 41.53 | 0.51 | 0.18 | 35.53 | 38.53 | 24.88 | |
| L318T | 0.63 | 0.15 | 23.67 | 0.59 | 0.12 | 19.51 | 21.59 | 40.64 | |
| D320E | 1.83 | 0.98 | 53.87 | 1.40 | 0.61 | 43.36 | 48.61 | 21.17 | |
| D320G | 1.16 | 0.22 | 18.74 | 0.88 | 0.17 | 19.09 | 18.91 | 40.64 | |
| D320H | 3.12 | 1.49 | 47.61 | 1.65 | 0.54 | 32.65 | 40.13 | 48.62 | |
| D320I | 1.28 | 0.39 | 30.80 | 1.24 | 0.37 | 30.02 | 30.41 | 40.64 | |
| D320K | 0.39 | 0.18 | 47.28 | 0.38 | 0.11 | 30.32 | 38.80 | 35.48 | |
| D320L | 0.36 | 0.16 | 44.62 | 0.38 | 0.13 | 34.21 | 39.41 | 21.17 | |
| D320M | 1.37 | 0.51 | 36.78 | 1.67 | 0.42 | 25.37 | 31.08 | 32.29 | |
| D320R | 4.11 | 2.64 | 64.27 | 2.08 | 1.13 | 54.42 | 59.34 | 48.62 | 305 |
| D320S | 1.76 | 0.69 | 39.30 | 1.43 | 0.45 | 31.22 | 35.26 | 21.17 | |
| D320V | 0.75 | 0.20 | 26.91 | 0.60 | 0.17 | 28.25 | 27.58 | 35.35 | |
| D320W | 0.32 | 0.09 | 29.02 | 0.33 | 0.08 | 25.61 | 27.31 | 37.77 | |
| D320Y | 0.61 | 0.23 | 37.61 | 0.56 | 0.12 | 22.21 | 29.91 | 21.17 | |
| N321A | 0.21 | 0.17 | 78.12 | 0.27 | 0.16 | 60.26 | 69.19 | 35.42 | 253 |
| N321H | 0.24 | 0.16 | 65.14 | 0.14 | 0.06 | 42.14 | 53.64 | 35.42 | 307 |
| N321R | 0.28 | 0.22 | 79.89 | 0.29 | 0.15 | 52.94 | 66.42 | 32.56 | 240 |
| N321S | 0.49 | 0.14 | 29.63 | 0.46 | 0.08 | 16.81 | 23.22 | 37.03 | |
| M323I | 0.69 | 0.13 | 19.25 | 0.71 | 0.13 | 18.36 | 18.80 | 37.03 | |
| M323L | 0.75 | 0.26 | 35.39 | 0.85 | 0.25 | 30.05 | 32.72 | 24.88 | |
| E324A | 0.48 | 0.24 | 49.84 | 0.45 | 0.13 | 28.13 | 38.99 | 32.56 | |
| E324D | 2.77 | 0.79 | 28.66 | 2.55 | 0.75 | 29.49 | 29.07 | 35.35 | |
| E324N | 1.57 | 0.69 | 44.27 | 1.54 | 0.59 | 38.35 | 41.31 | 32.56 | |
| E324R | 3.88 | 1.67 | 42.92 | 3.64 | 1.41 | 38.63 | 40.78 | 32.56 | |
| E324S | 1.13 | 0.38 | 33.75 | 1.32 | 0.44 | 32.97 | 33.36 | 24.88 | |
| T325E | 0.89 | 0.26 | 28.68 | 0.80 | 0.18 | 21.88 | 25.28 | 37.03 | |
| T325K | 1.56 | 0.48 | 30.76 | 1.55 | 0.58 | 37.17 | 33.96 | 20.98 | |
| T325N | 0.72 | 0.29 | 40.13 | 0.92 | 0.35 | 37.56 | 38.84 | 32.33 | |
| T325Q | 0.82 | 0.35 | 42.61 | 0.81 | 0.26 | 31.65 | 37.13 | 20.98 | |
| I326V | 1.49 | 0.57 | 38.48 | 2.01 | 0.62 | 31.02 | 34.75 | 32.33 | |
| L327M | 0.51 | 0.14 | 27.25 | 0.55 | 0.16 | 28.73 | 27.99 | 32.33 | |
| N328A | 0.70 | 0.34 | 47.97 | 0.67 | 0.27 | 40.37 | 44.17 | 32.33 | |
| N328C | 0.97 | 0.36 | 37.10 | 0.89 | 0.30 | 33.35 | 35.22 | 32.33 | |
| N328H | 0.61 | 0.20 | 33.20 | 0.72 | 0.29 | 39.54 | 36.37 | 20.98 | |
| N328I | 0.37 | 0.16 | 41.94 | 0.30 | 0.14 | 48.32 | 45.13 | 32.33 | |
| N328K | 1.36 | 0.52 | 38.20 | 1.48 | 0.55 | 37.42 | 37.81 | 20.98 | |
| N328L | 0.19 | 0.14 | 71.98 | 0.21 | 0.11 | 50.97 | 61.47 | 36.41 | 211 |
| N328Q | 0.76 | 0.35 | 46.23 | 0.68 | 0.21 | 30.49 | 38.36 | 35.48 | |
| N328R | 0.13 | 0.13 | 100.00 | 0.17 | 0.10 | 56.40 | 78.20 | 36.41 | 212 |
| N328T | 0.61 | 0.18 | 30.29 | 0.63 | 0.20 | 31.20 | 30.74 | 20.98 | |
| N328V | 0.76 | 0.25 | 33.33 | 0.79 | 0.22 | 27.74 | 30.54 | 20.98 | |
| N328Y | 1.39 | 0.46 | 32.79 | 1.30 | 0.35 | 26.96 | 29.88 | 20.98 | |
| I331V | 0.28 | 0.10 | 34.23 | 0.29 | 0.07 | 23.16 | 28.70 | 35.35 | |
| V334T | 0.43 | 0.10 | 22.83 | 0.36 | 0.06 | 17.79 | 20.31 | 21.17 | |
| K339M | 1.07 | 0.18 | 17.27 | 1.00 | 0.20 | 20.20 | 18.73 | 1.44 | |
| S342A | 1.93 | 1.99 | 103.00 | 1.69 | 2.09 | 124.13 | 113.57 | 1.44 | 97 |
| Q343V | 0.22 | 0.12 | 52.83 | 0.26 | 0.13 | 49.02 | 50.93 | 32.29 | 312 |
| Q347A | 2.18 | 0.92 | 41.96 | 2.60 | 0.72 | 27.87 | 34.91 | 35.35 | |
| Q347E | 6.15 | 0.97 | 15.81 | 5.59 | 2.75 | 49.28 | 32.54 | 7.03 | |
| Q347G | 2.76 | 1.01 | 36.48 | 2.89 | 0.81 | 28.14 | 32.31 | 35.35 | |
| Q347M | 1.86 | 0.35 | 18.91 | 3.77 | 1.15 | 30.39 | 24.65 | 7.03 | |
| Q347R | 1.76 | 0.40 | 22.66 | 2.56 | 0.45 | 17.40 | 20.03 | 7.03 | |
| Q347S | 2.62 | 0.34 | 13.01 | 3.51 | 0.55 | 15.77 | 14.39 | 7.03 | |
| E348D | 0.39 | 0.20 | 52.33 | 0.48 | 0.17 | 35.83 | 44.08 | 37.91 | |
| E348G | 1.65 | 0.33 | 20.00 | 1.69 | 0.34 | 20.32 | 20.16 | 7.03 | |
| E348S | 2.06 | 0.30 | 14.42 | 2.92 | 0.39 | 13.17 | 13.80 | 7.03 | |
| Q349A | 0.30 | 0.15 | 51.09 | 0.27 | 0.14 | 52.13 | 51.61 | 29.57 | 313 |
| Q349E | 0.27 | 0.16 | 58.75 | 0.33 | 0.14 | 41.85 | 50.30 | 29.57 | 314 |
| Q349K | 6.42 | 0.76 | 11.77 | 8.49 | 1.75 | 20.58 | 16.18 | 7.03 | |
| Q349M | 0.58 | 0.23 | 39.10 | 0.62 | 0.20 | 32.69 | 35.89 | 29.57 | |
| Q349R | 4.63 | 2.34 | 50.44 | 3.45 | 1.45 | 42.07 | 46.25 | 28.68 | |
| Q349T | 0.37 | 0.16 | 43.57 | 0.40 | 0.15 | 36.86 | 40.21 | 29.57 | |
| V351A | 3.22 | 1.43 | 44.33 | 2.63 | 0.77 | 29.43 | 36.88 | 28.68 | |
| V351C | 0.22 | 0.11 | 52.28 | 0.23 | 0.11 | 47.11 | 49.70 | 29.57 | |
| V351S | 1.17 | 0.59 | 50.19 | 1.02 | 0.32 | 31.76 | 40.98 | 28.68 | |
| I353T | 0.69 | 0.36 | 51.44 | 0.71 | 0.23 | 32.91 | 42.18 | 28.68 | |
| I353V | 4.90 | 2.29 | 46.67 | 2.94 | 0.99 | 33.61 | 40.14 | 28.68 | |
| N356D | 1.98 | 0.84 | 42.23 | 1.42 | 0.42 | 29.96 | 36.09 | 28.68 | |
| N356H | 2.24 | 0.73 | 32.60 | 2.09 | 0.69 | 33.15 | 32.88 | 28.68 | |
| S359D | 0.33 | 0.11 | 33.59 | 0.38 | 0.09 | 23.67 | 28.63 | 37.03 | |
| S359E | 3.20 | 2.11 | 65.89 | 2.23 | 0.92 | 41.15 | 53.52 | 28.68 | 315 |
| S359T | 0.84 | 0.41 | 48.57 | 0.83 | 0.30 | 35.75 | 42.16 | 37.18 | |
| S360T | 0.89 | 0.27 | 30.81 | 0.83 | 0.24 | 29.20 | 30.00 | 35.71 | |
| P367A | 0.54 | 0.13 | 24.21 | 0.46 | 0.10 | 21.82 | 23.02 | 37.03 | |
| P367G | 0.88 | 0.28 | 31.84 | 0.88 | 0.27 | 30.46 | 31.15 | 37.18 | |
| P367S | 1.66 | 0.48 | 29.07 | 1.61 | 0.42 | 26.02 | 27.55 | 35.71 | |
| D368A | 1.78 | 0.80 | 44.95 | 1.85 | 0.56 | 30.47 | 37.71 | 37.18 | |
| D368E | 2.15 | 0.84 | 38.96 | 1.86 | 0.64 | 34.51 | 36.73 | 37.18 | |
| D368H | 0.24 | 0.15 | 61.55 | 0.21 | 0.13 | 59.29 | 60.42 | 37.18 | 282 |
| D368K | 0.16 | 0.17 | 108.75 | 0.17 | 0.08 | 45.13 | 76.94 | 37.18 | 283 |
| D368L | 0.90 | 0.31 | 34.93 | 0.87 | 0.27 | 31.36 | 33.15 | 37.18 | |
| D368R | 1.74 | 0.59 | 33.76 | 2.38 | 1.08 | 45.43 | 39.59 | 30.11 | |
| D368T | 1.03 | 0.37 | 35.56 | 1.24 | 0.33 | 26.24 | 30.90 | 30.11 | |
| V058K | 14.14 | 8.25 | 58.34 | 6.88 | 4.25 | 61.77 | 60.06 | 11.45 | 383 |
| N369R | 2.93 | 0.86 | 29.31 | 2.62 | 0.75 | 28.66 | 28.98 | 35.71 | |
| N369S | 2.05 | 0.48 | 23.29 | 3.02 | 1.51 | 49.95 | 36.62 | 32.16 | |
| A371C | 0.38 | 0.12 | 31.79 | 0.33 | 0.10 | 30.94 | 31.36 | 19.08 | |
| A371E | 1.41 | 1.00 | 70.86 | 1.11 | 0.48 | 43.11 | 56.99 | 39.44 | 319 |
| A371F | 0.15 | 0.09 | 62.29 | 0.21 | 0.15 | 67.60 | 64.94 | 38.36 | 139 |
| A371G | 0.49 | 0.13 | 25.77 | 0.56 | 0.20 | 36.12 | 30.94 | 27.64 | |
| A371H | 0.64 | 0.25 | 38.25 | 0.77 | 0.24 | 31.66 | 34.95 | 27.64 | |
| A371I | 0.96 | 0.35 | 36.21 | 0.93 | 0.35 | 37.44 | 36.82 | 39.44 | |
| A371K | 0.83 | 0.32 | 37.79 | 0.78 | 0.26 | 33.42 | 35.61 | 27.64 | |
| A371L | 0.43 | 0.07 | 15.54 | 0.50 | 0.07 | 14.86 | 15.20 | 35.71 | |
| A371L | 0.48 | 0.16 | 32.95 | 0.53 | 0.16 | 30.92 | 31.94 | 27.64 | |
| A371M | 0.79 | 0.47 | 59.82 | 0.71 | 0.35 | 49.93 | 54.88 | 35.71 | 321 |
| A371R | 0.86 | 0.42 | 48.51 | 0.92 | 0.40 | 43.35 | 45.93 | 39.44 | |
| A371S | 0.66 | 0.25 | 38.44 | 0.72 | 0.30 | 41.65 | 40.04 | 39.44 | |
| A371T | 0.34 | 0.18 | 53.34 | 0.20 | 0.12 | 59.46 | 56.40 | 37.23 | 322 |
| A371V | 0.83 | 0.26 | 30.97 | 1.00 | 0.26 | 25.74 | 28.35 | 39.44 | |
| Q373A | 0.72 | 0.16 | 22.25 | 0.66 | 0.18 | 27.32 | 24.79 | 19.08 | |
| Q373E | 2.39 | 0.28 | 11.49 | 3.70 | 1.34 | 36.26 | 23.88 | 39.44 | |
| Q373F | 0.54 | 0.20 | 37.44 | 0.57 | 0.19 | 34.04 | 35.74 | 27.64 | |
| Q373K | 0.98 | 0.37 | 37.77 | 0.95 | 0.31 | 32.37 | 35.07 | 27.64 | |
| Q373R | 0.51 | 0.16 | 30.78 | 0.44 | 0.24 | 55.25 | 43.01 | 28.92 | |
| Q373S | 0.23 | 0.12 | 52.88 | 0.23 | 0.22 | 96.90 | 74.89 | 28.92 | 214 |
| Q373V | 1.55 | 0.55 | 35.47 | 1.55 | 0.59 | 38.08 | 36.77 | 28.92 | |
| L374A | 0.39 | 0.28 | 70.56 | 0.79 | 0.30 | 37.37 | 53.96 | 28.92 | 324 |
| L374H | 1.17 | 0.41 | 35.21 | 1.27 | 0.41 | 32.06 | 33.63 | 27.64 | |
| L374I | 1.01 | 0.29 | 28.98 | 1.08 | 0.30 | 28.16 | 28.57 | 37.41 | |
| L374M | 1.22 | 0.52 | 42.66 | 1.21 | 0.46 | 37.95 | 40.31 | 27.64 | |
| L374N | 0.65 | 0.20 | 31.16 | 0.74 | 0.20 | 27.38 | 29.27 | 37.41 | |
| L374P | 0.56 | 0.15 | 27.74 | 0.61 | 0.17 | 27.30 | 27.52 | 37.41 | |
| L374R | 0.30 | 0.15 | 48.59 | 0.30 | 0.12 | 39.83 | 44.21 | 37.41 | |
| L374S | 0.56 | 0.24 | 41.87 | 0.63 | 0.19 | 30.18 | 36.02 | 37.41 | |
| L374T | 0.36 | 0.11 | 29.89 | 0.37 | 0.10 | 27.41 | 28.65 | 37.41 | |
| L374V | 0.19 | 0.13 | 65.71 | 0.23 | 0.10 | 42.42 | 54.07 | 37.41 | 325 |
| L374W | 0.63 | 0.20 | 31.65 | 0.74 | 0.18 | 24.73 | 28.19 | 27.20 | |
| L374Y | 1.66 | 0.46 | 27.62 | 1.92 | 0.56 | 29.12 | 28.37 | 27.20 | |
| E375G | 1.85 | 0.61 | 32.88 | 2.04 | 0.53 | 25.80 | 29.34 | 30.11 | |
| E375I | 0.29 | 0.12 | 41.98 | 0.28 | 0.09 | 32.01 | 36.99 | 19.08 | |
| E375L | 0.87 | 0.31 | 34.88 | 0.91 | 0.24 | 26.70 | 30.79 | 30.11 | |
| E375M | 0.87 | 0.30 | 34.97 | 0.84 | 0.23 | 27.56 | 31.27 | 30.11 | |
| E375N | 1.46 | 0.49 | 33.28 | 1.49 | 0.43 | 28.95 | 31.11 | 30.11 | |
| E375R | 1.56 | 0.39 | 25.28 | 1.65 | 0.33 | 19.83 | 22.56 | 35.71 | |
| E375S | 0.66 | 0.25 | 37.57 | 0.82 | 0.13 | 16.21 | 26.89 | 30.11 | |
| E375T | 3.73 | 1.99 | 53.47 | 3.41 | 2.09 | 61.19 | 57.33 | 30.11 | 326 |
| K376A | 1.15 | 0.30 | 26.41 | 1.77 | 0.57 | 31.89 | 29.15 | 29.20 | |
| K376D | 1.71 | 0.45 | 26.05 | 2.01 | 0.44 | 22.03 | 24.04 | 29.20 | |
| K376E | 2.17 | 0.61 | 28.10 | 2.56 | 0.52 | 20.09 | 24.09 | 29.20 | |
| K376Q | 1.18 | 0.26 | 22.10 | 1.33 | 0.30 | 22.34 | 22.22 | 29.20 | |
| K376R | 0.93 | 0.20 | 21.30 | 0.94 | 0.15 | 16.27 | 18.78 | 29.20 | |
| K376S | 1.11 | 0.24 | 21.48 | 0.96 | 0.16 | 16.89 | 19.19 | 29.20 | |
| K376T | 1.40 | 0.25 | 17.58 | 1.59 | 0.29 | 18.38 | 17.98 | 29.20 | |
| K376Y | 1.55 | 0.81 | 51.93 | 1.56 | 0.88 | 56.60 | 54.27 | 27.93 | 327 |
| G377D | 3.19 | 1.26 | 39.34 | 3.90 | 1.40 | 35.87 | 37.60 | 27.93 | |
| G377E | 1.24 | 0.43 | 35.03 | 1.53 | 0.40 | 26.02 | 30.53 | 27.93 | |
| G377H | 1.52 | 0.49 | 32.45 | 1.55 | 0.51 | 33.05 | 32.75 | 27.93 | |
| G377K | 1.95 | 0.63 | 32.52 | 1.80 | 0.59 | 32.87 | 32.70 | 27.93 | |
| G377P | 0.81 | 0.21 | 26.05 | 0.82 | 0.28 | 33.96 | 30.01 | 27.93 | |
| G377R | 2.83 | 0.99 | 35.07 | 3.02 | 0.91 | 30.00 | 32.53 | 27.93 | |
| G377S | 6.11 | 2.61 | 42.74 | 6.73 | 2.55 | 37.91 | 40.32 | 27.93 | |
| G377T | 6.31 | 5.13 | 81.24 | 4.15 | 2.02 | 48.79 | 65.01 | 30.28 | 270 |
| G378K | 1.04 | 0.34 | 33.16 | 1.34 | 0.75 | 56.00 | 44.58 | 30.28 | |
| G378R | 2.10 | 0.62 | 29.46 | 2.65 | 1.02 | 38.40 | 33.93 | 30.28 | |
| K379H | 1.49 | 1.54 | 103.18 | 4.72 | 4.14 | 87.65 | 95.42 | 31.91 | 118 |
| K379R | 0.13 | 0.06 | 44.19 | 0.15 | 0.01 | 8.50 | 26.35 | 35.71 | |
| K379S | 0.35 | 0.26 | 73.32 | 0.73 | 1.13 | 155.75 | 114.54 | 31.91 | 187 |
| K379T | 0.57 | 0.35 | 61.16 | 1.18 | 1.27 | 107.72 | 84.44 | 31.91 | 178 |
| F380I | 0.41 | 0.27 | 66.18 | 0.98 | 0.91 | 93.03 | 79.60 | 31.91 | 193 |
| L165F | 1.40 | 1.23 | 88.06 | 1.29 | 1.09 | 84.44 | 86.25 | 24.02 | 171 |
| F380P | 0.39 | 0.30 | 75.10 | 0.97 | 0.95 | 98.50 | 86.80 | 31.91 | 192 |
| F380T | 0.59 | 0.40 | 67.20 | 1.14 | 1.62 | 142.48 | 104.84 | 31.91 | 113 |
| F380V | 1.52 | 0.97 | 64.19 | 0.90 | 0.38 | 42.73 | 53.46 | 24.26 | 336 |
| F380W | 14.68 | 3.34 | 22.78 | 12.12 | 2.54 | 20.91 | 21.85 | 24.26 | |
| F380Y | 3.42 | 1.26 | 36.74 | 3.11 | 0.87 | 27.96 | 32.35 | 24.26 | |
| T381E | 0.92 | 0.32 | 34.75 | 0.90 | 0.22 | 24.35 | 29.55 | 24.26 | |
| T381H | 1.34 | 0.49 | 36.24 | 1.17 | 0.29 | 24.39 | 30.31 | 24.26 | |
| T381K | 0.21 | 0.10 | 44.96 | 0.23 | 0.10 | 41.04 | 43.00 | 27.20 | |
| T381N | 1.42 | 0.48 | 33.74 | 1.11 | 0.29 | 26.20 | 29.97 | 24.26 | |
| T381Q | 1.90 | 0.70 | 36.68 | 1.65 | 0.45 | 27.19 | 31.94 | 24.26 | |
| T381R | 3.51 | 1.54 | 43.88 | 2.23 | 0.98 | 43.89 | 43.88 | 24.26 | |
| T381S | 2.56 | 1.12 | 43.76 | 2.51 | 1.02 | 40.50 | 42.13 | 26.26 | |
| T381V | 1.78 | 0.60 | 33.71 | 1.57 | 0.48 | 30.78 | 32.24 | 26.26 | |
| R383A | 0.79 | 0.21 | 27.00 | 0.79 | 0.18 | 22.95 | 24.97 | 26.26 | |
| R383E | 0.76 | 0.20 | 25.92 | 0.74 | 0.13 | 18.05 | 21.98 | 26.26 | |
| R383H | 1.01 | 0.33 | 33.00 | 1.13 | 0.30 | 26.06 | 29.53 | 27.20 | |
| R383I | 0.81 | 0.24 | 29.16 | 0.96 | 0.27 | 28.41 | 28.79 | 36.38 | |
| R383K | 0.65 | 0.22 | 33.49 | 0.64 | 0.15 | 23.26 | 28.37 | 26.26 | |
| R383M | 0.79 | 0.19 | 24.21 | 0.67 | 0.17 | 25.56 | 24.88 | 26.26 | |
| R383N | 1.05 | 0.30 | 28.31 | 0.96 | 0.28 | 29.30 | 28.80 | 26.26 | |
| R383S | 3.80 | 1.89 | 49.91 | 2.49 | 0.72 | 28.89 | 39.40 | 27.07 | |
| R383T | 2.55 | 0.49 | 19.02 | 2.40 | 0.57 | 23.71 | 21.37 | 27.07 | |
| R383V | 1.89 | 0.48 | 25.43 | 2.09 | 0.41 | 19.57 | 22.50 | 27.07 | |
| K385A | 0.50 | 0.00 | 0.00 | 0.64 | 0.00 | 0.00 | 0.00 | 0.00 | |
| K385Q | 1.26 | 0.00 | 0.00 | 1.50 | 0.03 | 1.73 | 0.86 | 0.00 | |
| K385R | 1.22 | 0.00 | 0.00 | 1.36 | 0.01 | 0.44 | 0.22 | 0.00 | |
| K385S | 0.78 | 0.00 | 0.00 | 0.80 | 0.00 | 0.00 | 0.00 | 0.00 | |
| T387S | 0.78 | 0.24 | 31.06 | 0.55 | 0.20 | 35.41 | 33.23 | 18.12 | |
| L388F | 0.48 | 0.23 | 48.90 | 0.69 | 0.28 | 39.73 | 44.31 | 37.83 | |
| L388H | 0.14 | 0.15 | 104.56 | 0.33 | 0.23 | 68.33 | 86.44 | 26.52 | 351 |
| L388I | 2.15 | 0.64 | 29.79 | 1.95 | 0.75 | 38.62 | 34.20 | 26.25 | |
| L388M | 0.48 | 0.19 | 39.25 | 0.28 | 0.14 | 50.44 | 44.85 | 39.97 | |
| L388R | 0.63 | 0.18 | 28.22 | 0.69 | 0.19 | 27.12 | 27.67 | 27.20 | |
| L388T | 0.38 | 0.18 | 46.48 | 0.44 | 0.18 | 40.07 | 43.28 | 28.63 | |
| L388V | 1.07 | 0.35 | 32.57 | 0.82 | 0.28 | 34.27 | 33.42 | 39.97 | |
| L388W | 0.47 | 0.19 | 39.35 | 0.46 | 0.18 | 37.86 | 38.61 | 37.53 | |
| L388Y | 1.60 | 0.20 | 12.55 | 1.61 | 0.36 | 22.59 | 17.57 | 39.97 | |
| E389A | 0.94 | 0.42 | 44.66 | 1.36 | 0.49 | 36.16 | 40.41 | 28.26 | |
| E389G | 0.35 | 0.17 | 47.41 | 0.38 | 0.18 | 47.75 | 47.58 | 34.42 | |
| E389H | 1.29 | 0.35 | 27.05 | 1.06 | 0.27 | 25.25 | 26.15 | 17.14 | |
| E389K | 0.25 | 0.14 | 58.15 | 0.22 | 0.13 | 61.61 | 59.88 | 17.14 | 338 |
| E389L | 0.61 | 0.20 | 32.76 | 0.57 | 0.20 | 35.11 | 33.93 | 34.42 | |
| E389M | 0.41 | 0.12 | 29.39 | 0.41 | 0.14 | 34.65 | 32.02 | 19.08 | |
| E389Q | 0.81 | 0.25 | 31.08 | 0.92 | 0.20 | 22.23 | 26.66 | 19.08 | |
| E389R | 0.87 | 0.35 | 39.86 | 0.81 | 0.29 | 35.82 | 37.84 | 28.26 | |
| E392A | 0.59 | 0.00 | 0.00 | 0.68 | 0.00 | 0.00 | 0.00 | 0.00 | |
| E392F | 0.27 | 0.00 | 0.00 | 0.37 | 0.00 | 0.00 | 0.00 | 0.00 | |
| E392K | 0.46 | 0.00 | 0.00 | 0.67 | 0.00 | 0.00 | 0.00 | 0.00 | |
| E392L | 3.36 | 1.48 | 43.94 | 1.84 | 0.72 | 38.89 | 41.42 | 9.66 | |
| E392M | 2.31 | 0.89 | 38.47 | 1.74 | 0.49 | 28.10 | 33.29 | 9.66 | |
| E392Q | 2.64 | 1.14 | 43.29 | 1.59 | 0.50 | 31.31 | 37.30 | 9.66 | |
| E392R | 1.62 | 0.56 | 34.26 | 1.05 | 0.31 | 29.77 | 32.01 | 9.66 | |
| E392S | 0.82 | 0.32 | 38.96 | 0.63 | 0.18 | 28.53 | 33.74 | 9.66 | |
| E392T | 1.28 | 0.49 | 38.26 | 0.91 | 0.26 | 28.51 | 33.38 | 9.66 | |
| E392V | 2.09 | 0.65 | 31.09 | 1.15 | 0.30 | 26.35 | 28.72 | 9.66 | |
| E392Y | 2.98 | 1.14 | 38.12 | 1.54 | 0.48 | 30.93 | 34.52 | 9.66 | |
| Q393F | 0.33 | 0.12 | 36.46 | 0.28 | 0.10 | 34.51 | 35.49 | 27.93 | |
| Q393H | 1.70 | 0.58 | 34.23 | 1.62 | 0.07 | 4.27 | 19.25 | 17.14 | |
| Q393L | 0.42 | 0.26 | 61.83 | 0.48 | 0.19 | 40.06 | 50.95 | 17.14 | 339 |
| Q393N | 1.19 | 0.37 | 31.42 | 1.61 | 0.31 | 19.23 | 25.32 | 27.20 | |
| Q393S | 0.41 | 0.11 | 26.92 | 0.12 | 0.00 | 2.59 | 14.75 | 26.25 | |
| Q393T | 0.19 | 0.08 | 41.93 | 0.21 | 0.06 | 30.12 | 36.02 | 27.93 | |
| S395A | 0.98 | 0.24 | 24.27 | 0.73 | 0.14 | 19.14 | 21.71 | 4.00 | |
| S395H | 0.77 | 0.20 | 26.31 | 0.79 | 0.25 | 31.78 | 29.05 | 27.93 | |
| S395K | 2.67 | 0.77 | 29.01 | 1.85 | 0.52 | 28.15 | 28.58 | 4.00 | |
| S395R | 1.25 | 0.36 | 28.94 | 1.04 | 0.25 | 24.31 | 26.63 | 4.00 | |
| S395T | 0.27 | 0.10 | 35.35 | 0.29 | 0.10 | 35.75 | 35.55 | 27.93 | |
| E396A | 0.27 | 0.10 | 36.60 | 0.22 | 0.02 | 9.55 | 23.07 | 4.00 | |
| E396D | 0.89 | 0.26 | 29.76 | 0.84 | 0.14 | 16.85 | 23.30 | 4.00 | |
| E396H | 0.52 | 0.11 | 20.73 | 0.45 | 0.03 | 7.24 | 13.98 | 4.00 | |
| E396L | 0.81 | 0.19 | 23.66 | 0.67 | 0.13 | 19.69 | 21.67 | 4.00 | |
| E396Q | 0.90 | 0.25 | 27.13 | 0.96 | 0.22 | 23.19 | 25.16 | 27.93 | |
| E396R | 1.15 | 0.33 | 29.01 | 0.80 | 0.26 | 31.88 | 30.44 | 19.42 | |
| E396S | 1.03 | 0.33 | 32.21 | 0.99 | 0.28 | 28.48 | 30.35 | 19.42 | |
| E396T | 0.76 | 0.23 | 30.01 | 0.63 | 0.20 | 32.22 | 31.12 | 19.42 | |
| Y399E | 3.58 | 1.18 | 32.85 | 3.46 | 1.08 | 31.26 | 32.06 | 17.14 | |
| Y399K | 5.34 | 1.86 | 34.76 | 4.78 | 1.63 | 34.18 | 34.47 | 17.14 | |
| Y399M | 1.60 | 0.82 | 51.25 | 1.73 | 0.82 | 47.37 | 49.31 | 34.83 | |
| Y399Q | 1.09 | 0.46 | 42.63 | 1.26 | 0.64 | 51.03 | 46.83 | 31.17 | |
| Y399R | 0.65 | 0.33 | 50.81 | 0.60 | 0.29 | 49.24 | 50.03 | 34.83 | 340 |
| Y399S | 0.98 | 0.39 | 40.11 | 0.84 | 0.34 | 40.24 | 40.18 | 34.83 | |
| Y399T | 2.80 | 1.00 | 35.61 | 2.42 | 1.01 | 41.52 | 38.56 | 34.83 | |
| Y399V | 2.51 | 0.69 | 27.54 | 2.65 | 0.73 | 27.39 | 27.47 | 22.91 | |
| Y399W | 0.32 | 0.20 | 62.24 | 0.46 | 0.21 | 45.03 | 53.64 | 31.17 | 341 |
| S401A | 1.55 | 0.06 | 3.56 | 1.55 | 0.03 | 1.96 | 2.76 | 19.42 | |
| S401E | 1.00 | 0.33 | 32.52 | 0.88 | 0.26 | 30.02 | 31.27 | 19.42 | |
| S401G | 0.17 | 0.11 | 62.94 | 0.18 | 0.09 | 48.60 | 55.77 | 19.42 | 342 |
| S401N | 1.97 | 0.30 | 15.46 | 1.14 | 0.31 | 26.91 | 21.18 | 22.91 | |
| Y403F | 0.50 | 0.30 | 59.08 | 0.58 | 0.26 | 44.25 | 51.67 | 34.83 | 343 |
| T405K | 0.66 | 0.30 | 44.65 | 0.51 | 0.21 | 41.63 | 43.14 | 35.45 | |
| T405M | 1.19 | 0.48 | 40.05 | 1.48 | 0.60 | 40.56 | 40.31 | 24.78 | |
| T405R | 0.66 | 0.24 | 35.95 | 0.70 | 0.28 | 40.50 | 38.22 | 24.78 | |
| T405S | 1.10 | 0.33 | 29.58 | 1.66 | 0.53 | 32.12 | 30.85 | 22.91 | |
| L406A | 0.73 | 0.27 | 37.06 | 0.62 | 0.23 | 36.93 | 37.00 | 28.63 | |
| L406E | 1.28 | 0.37 | 28.99 | 1.30 | 0.38 | 29.35 | 29.17 | 39.97 | |
| L406F | 0.34 | 0.14 | 42.39 | 0.24 | 0.11 | 45.17 | 43.78 | 39.97 | |
| L406G | 2.34 | 0.87 | 37.12 | 1.38 | 0.02 | 1.52 | 19.32 | 39.97 | |
| L406I | 0.94 | 0.35 | 36.78 | 0.65 | 0.32 | 48.66 | 42.72 | 28.63 | |
| L406N | 0.26 | 0.20 | 74.38 | 0.32 | 0.20 | 63.48 | 68.93 | 23.96 | 317 |
| L406Q | 0.68 | 0.19 | 27.68 | 1.50 | 0.38 | 25.13 | 26.40 | 22.91 | |
| L406S | 0.17 | 0.09 | 48.85 | 0.17 | 0.08 | 50.30 | 49.57 | 30.48 | |
| L406T | 0.84 | 0.23 | 27.11 | 0.77 | 0.27 | 34.79 | 30.95 | 30.48 | |
| L406V | 0.65 | 0.33 | 50.23 | 0.70 | 0.30 | 43.29 | 46.76 | 23.96 | |
| S407A | 0.19 | 0.04 | 18.62 | 0.17 | 0.01 | 5.56 | 12.09 | 35.45 | |
| S407D | 0.83 | 0.42 | 51.18 | 0.86 | 0.38 | 44.54 | 47.86 | 37.88 | |
| S407E | 0.43 | 0.17 | 38.75 | 0.49 | 0.17 | 35.56 | 37.15 | 31.75 | |
| S407F | 0.27 | 0.18 | 68.03 | 0.25 | 0.15 | 58.65 | 63.34 | 23.96 | 318 |
| S407H | 0.31 | 0.18 | 59.55 | 0.29 | 0.14 | 46.52 | 53.03 | 23.96 | 346 |
| S407L | 0.58 | 0.25 | 42.45 | 0.41 | 0.19 | 44.87 | 43.66 | 30.53 | |
| S407M | 0.36 | 0.24 | 66.57 | 0.51 | 0.20 | 38.54 | 52.56 | 31.75 | 347 |
| S407N | 0.78 | 0.20 | 25.66 | 1.27 | 0.34 | 27.14 | 26.40 | 22.91 | |
| S407P | 0.23 | 0.15 | 64.97 | 0.27 | 0.09 | 35.14 | 50.05 | 31.75 | 348 |
| S407Q | 0.20 | 0.16 | 77.17 | 0.28 | 0.12 | 43.68 | 60.43 | 31.75 | 198 |
| S407R | 0.53 | 0.26 | 48.77 | 0.48 | 0.20 | 41.05 | 44.91 | 23.96 | |
| K409E | 0.94 | 0.42 | 44.76 | 1.00 | 0.45 | 44.79 | 44.78 | 27.54 | |
| K409H | 0.49 | 0.11 | 23.26 | 0.85 | 0.23 | 27.04 | 25.15 | 22.91 | |
| K409Q | 1.10 | 0.46 | 41.72 | 0.95 | 0.32 | 33.65 | 37.68 | 33.75 | |
| K409S | 1.66 | 0.60 | 36.18 | 2.11 | 0.96 | 45.48 | 40.83 | 27.93 | |
| K409T | 1.43 | 0.57 | 39.90 | 0.96 | 0.36 | 38.00 | 38.95 | 33.75 | |
| E410D | 0.27 | 0.03 | 12.10 | 0.67 | 0.09 | 13.88 | 12.99 | 22.91 | |
| E410M | 0.46 | 0.23 | 51.20 | 0.45 | 0.19 | 43.37 | 47.29 | 31.17 | |
| E410P | 0.91 | 0.41 | 44.74 | 0.59 | 0.20 | 34.01 | 39.38 | 33.75 | |
| E410S | 2.11 | 1.60 | 76.06 | 1.79 | 1.09 | 60.69 | 68.37 | 33.75 | 230 |
| E410T | 2.89 | 1.20 | 41.61 | 3.32 | 1.26 | 37.81 | 39.71 | 32.12 | |
| K411A | 0.18 | 0.08 | 43.34 | 0.18 | 0.06 | 32.05 | 37.70 | 37.77 | |
| K411H | 0.23 | 0.08 | 34.21 | 0.23 | 0.05 | 21.51 | 27.86 | 37.77 | |
| K411N | 0.58 | 0.22 | 37.93 | 0.61 | 0.20 | 32.13 | 35.03 | 37.77 | |
| K411P | 0.51 | 0.13 | 25.83 | 0.43 | 0.12 | 27.58 | 26.71 | 51.63 | |
| K411R | 0.45 | 0.13 | 28.82 | 0.39 | 0.12 | 29.63 | 29.23 | 51.63 | |
| K411S | 0.69 | 0.21 | 29.80 | 0.54 | 0.16 | 30.26 | 30.03 | 51.63 | |
| K411T | 0.87 | 0.34 | 39.20 | 0.77 | 0.22 | 28.48 | 33.84 | 51.63 | |
| K411V | 0.69 | 0.29 | 41.46 | 0.64 | 0.22 | 34.61 | 38.03 | 51.63 | |
| A412D | 1.24 | 0.54 | 43.52 | 1.54 | 0.53 | 34.13 | 38.83 | 31.75 | |
| A412G | 0.15 | 0.09 | 61.17 | 0.24 | 0.28 | 113.76 | 87.46 | 29.74 | 296 |
| A412H | 0.70 | 0.14 | 19.89 | 0.87 | 0.13 | 14.67 | 17.28 | 31.98 | |
| A412I | 0.17 | 0.04 | 24.93 | 0.51 | 0.27 | 53.75 | 39.34 | 29.74 | |
| A412L | 0.57 | 0.27 | 47.33 | 1.34 | 0.62 | 46.19 | 46.76 | 29.74 | |
| A412N | 0.45 | 0.20 | 45.58 | 0.49 | 0.19 | 39.84 | 42.71 | 31.17 | |
| A412P | 0.16 | 0.14 | 86.25 | 0.19 | 0.14 | 77.07 | 81.66 | 28.04 | 244 |
| A412Q | 0.71 | 0.23 | 31.77 | 1.74 | 0.82 | 47.41 | 39.59 | 29.74 | |
| A412R | 0.95 | 0.27 | 28.98 | 0.97 | 0.27 | 28.15 | 28.57 | 34.42 | |
| D284S | 0.31 | 0.37 | 119.17 | 0.35 | 0.41 | 116.31 | 117.74 | 37.53 | 136 |
| A412V | 0.49 | 0.23 | 46.42 | 0.32 | 0.15 | 45.99 | 46.20 | 30.53 | |
| A412W | 0.44 | 0.18 | 41.42 | 0.51 | 0.20 | 38.89 | 40.15 | 34.42 | |
| A412Y | 0.51 | 0.21 | 41.01 | 0.55 | 0.20 | 36.80 | 38.91 | 34.42 | |
| D413K | 0.45 | 0.19 | 40.93 | 0.54 | 0.15 | 26.98 | 33.95 | 34.42 | |
| D413N | 0.73 | 0.19 | 25.54 | 1.18 | 0.28 | 23.95 | 24.75 | 22.91 | |
| D413Q | 0.15 | 0.11 | 76.71 | 0.23 | 0.15 | 64.67 | 70.69 | 27.59 | 260 |
| D413R | 0.46 | 0.15 | 33.41 | 0.43 | 0.14 | 32.29 | 32.85 | 34.42 | |
| D413S | 0.90 | 0.36 | 39.84 | 0.90 | 0.36 | 40.51 | 40.18 | 27.87 | |
| D413T | 0.44 | 0.19 | 43.23 | 0.25 | 0.17 | 70.16 | 56.69 | 27.87 | 354 |
| V414I | 0.53 | 0.20 | 36.52 | 0.57 | 0.20 | 34.65 | 35.58 | 32.12 | |
| K415G | 0.39 | 0.23 | 58.83 | 0.67 | 0.21 | 31.73 | 45.28 | 27.59 | |
| K415W | 0.33 | 0.20 | 61.03 | 0.50 | 0.20 | 40.42 | 50.72 | 27.59 | 355 |
| D416F | 0.39 | 0.18 | 46.73 | 0.29 | 0.06 | 22.63 | 34.68 | 21.17 | |
| D416G | 0.38 | 0.11 | 28.25 | 0.31 | 0.08 | 26.31 | 27.28 | 21.17 | |
| D416H | 0.19 | 0.08 | 39.63 | 0.22 | 0.12 | 55.02 | 47.33 | 24.69 | |
| D416I | 0.54 | 0.13 | 23.86 | 0.64 | 0.20 | 31.62 | 27.74 | 31.57 | |
| D416K | 1.11 | 0.45 | 40.39 | 0.89 | 0.27 | 30.26 | 35.33 | 21.17 | |
| D416L | 1.03 | 0.46 | 44.80 | 1.06 | 0.42 | 39.24 | 42.02 | 29.17 | |
| D416N | 0.88 | 0.29 | 32.97 | 0.76 | 0.20 | 25.94 | 29.46 | 31.91 | |
| D416Q | 0.30 | 0.08 | 26.07 | 0.25 | 0.06 | 23.27 | 24.67 | 31.77 | |
| D416R | 0.17 | 0.10 | 56.18 | 0.20 | 0.09 | 43.51 | 49.84 | 29.17 | |
| T417I | 0.81 | 0.32 | 39.06 | 0.77 | 0.25 | 33.01 | 36.04 | 31.77 | |
| T417L | 0.28 | 0.16 | 56.63 | 0.28 | 0.13 | 45.71 | 51.17 | 29.17 | 356 |
| D418A | 1.14 | 0.30 | 26.74 | 0.90 | 0.34 | 37.60 | 32.17 | 29.17 | |
| D418E | 0.48 | 0.17 | 35.19 | 0.42 | 0.15 | 35.06 | 35.12 | 31.77 | |
| D418F | 0.64 | 0.22 | 35.04 | 0.57 | 0.18 | 30.95 | 33.00 | 31.91 | |
| D418G | 0.44 | 0.14 | 32.80 | 0.46 | 0.18 | 38.41 | 35.61 | 29.17 | |
| D418I | 1.84 | 0.48 | 26.06 | 2.38 | 0.77 | 32.14 | 29.10 | 31.57 | |
| D418L | 0.67 | 0.24 | 35.78 | 0.72 | 0.25 | 35.00 | 35.39 | 29.17 | |
| D418M | 1.08 | 0.38 | 35.22 | 0.95 | 0.32 | 33.23 | 34.22 | 29.17 | |
| D418N | 1.26 | 0.42 | 33.25 | 1.08 | 0.38 | 34.75 | 34.00 | 26.51 | |
| D418P | 0.65 | 0.22 | 34.31 | 0.72 | 0.25 | 35.12 | 34.71 | 26.51 | |
| D418Q | 1.11 | 0.37 | 33.36 | 1.28 | 0.37 | 29.11 | 31.24 | 26.51 | |
| D418R | 1.13 | 0.35 | 30.93 | 1.45 | 0.35 | 24.38 | 27.66 | 26.51 | |
| D418S | 0.76 | 0.25 | 32.37 | 0.78 | 0.20 | 26.13 | 29.25 | 26.51 | |
| D418V | 1.26 | 0.41 | 32.74 | 1.53 | 0.37 | 24.37 | 28.56 | 26.51 | |
| D418Y | 0.82 | 0.35 | 43.19 | 0.87 | 0.26 | 30.11 | 36.65 | 26.51 | |
| A419E | 0.23 | 0.04 | 17.14 | 0.30 | 0.05 | 16.06 | 16.60 | 31.57 | |
| A419F | 0.58 | 0.23 | 40.03 | 0.72 | 0.38 | 52.39 | 46.21 | 30.10 | |
| A419G | 0.56 | 0.24 | 42.55 | 0.58 | 0.21 | 36.96 | 39.75 | 27.87 | |
| A419H | 4.24 | 1.00 | 23.60 | 3.96 | 0.88 | 22.11 | 22.85 | 31.91 | |
| A419I | 0.99 | 0.39 | 39.55 | 1.36 | 0.53 | 39.37 | 39.46 | 30.10 | |
| A419K | 6.23 | 1.90 | 30.53 | 11.48 | 3.06 | 26.69 | 28.61 | 30.10 | |
| A419L | 0.49 | 0.19 | 37.91 | 0.38 | 0.26 | 68.32 | 53.12 | 26.51 | 357 |
| A419R | 2.80 | 1.09 | 38.94 | 3.35 | 1.18 | 35.14 | 37.04 | 24.88 | |
| A419S | 4.61 | 2.24 | 48.60 | 2.24 | 0.82 | 36.51 | 42.55 | 34.49 | |
| A419W | 1.65 | 0.66 | 40.25 | 0.89 | 0.29 | 32.90 | 36.58 | 34.49 | |
| A419Y | 4.84 | 1.76 | 36.32 | 3.39 | 1.00 | 29.54 | 32.93 | 34.49 | |
| V420I | 0.19 | 0.04 | 19.85 | 0.15 | 0.03 | 18.18 | 19.01 | 34.49 | |
| D421A | 2.47 | 0.91 | 36.74 | 1.82 | 0.57 | 31.22 | 33.98 | 34.49 | |
| D421E | 1.68 | 0.61 | 36.08 | 1.03 | 0.30 | 29.40 | 32.74 | 34.49 | |
| D421G | 0.73 | 0.34 | 45.87 | 0.54 | 0.16 | 29.45 | 37.66 | 34.49 | |
| D421H | 6.72 | 1.61 | 23.99 | 9.33 | 2.50 | 26.76 | 25.37 | 31.57 | |
| D421I | 0.65 | 0.44 | 66.54 | 0.32 | 0.15 | 45.75 | 56.14 | 34.49 | 358 |
| D421K | 0.24 | 0.08 | 32.36 | 0.20 | 0.09 | 46.63 | 39.50 | 31.00 | |
| D421L | 1.48 | 0.45 | 30.17 | 1.67 | 0.75 | 45.16 | 37.66 | 31.57 | |
| D421M | 0.33 | 0.07 | 22.09 | 0.11 | 0.11 | 100.44 | 61.26 | 31.57 | 271 |
| D421N | 0.99 | 0.43 | 43.86 | 0.67 | 0.28 | 41.82 | 42.84 | 32.10 | |
| D421Q | 2.08 | 0.65 | 31.30 | 1.66 | 0.57 | 34.36 | 32.83 | 31.00 | |
| D421R | 0.11 | 0.06 | 56.76 | 0.11 | 0.03 | 30.66 | 43.71 | 31.00 | |
| D421T | 1.88 | 0.75 | 39.64 | 1.84 | 0.49 | 26.55 | 33.09 | 16.69 | |
| D421Y | 0.87 | 0.19 | 21.53 | 0.86 | 0.07 | 8.28 | 14.90 | 16.69 | |
| V422I | 0.19 | 0.04 | 20.64 | 0.19 | 0.04 | 18.35 | 19.49 | 31.00 | |
| A425G | 3.16 | 0.72 | 22.83 | 3.83 | 1.11 | 29.00 | 25.91 | 14.50 | |
| A425K | 0.18 | 0.06 | 33.97 | 0.18 | 0.05 | 28.46 | 31.21 | 31.91 | |
| A425M | 0.94 | 0.42 | 44.06 | 0.80 | 0.27 | 34.02 | 39.04 | 16.69 | |
| A425N | 0.15 | 0.00 | 0.00 | 0.14 | 0.00 | 0.00 | 0.00 | 14.50 | |
| A425S | 0.30 | 0.01 | 1.83 | 0.38 | 0.00 | 0.00 | 0.91 | 14.50 | |
| D426E | 1.36 | 0.51 | 37.70 | 0.95 | 0.29 | 30.07 | 33.89 | 32.10 | |
| D426G | 0.60 | 0.01 | 1.50 | 0.69 | 0.05 | 6.89 | 4.20 | 14.50 | |
| D426K | 0.51 | 0.04 | 6.85 | 0.51 | 0.00 | 0.00 | 3.42 | 14.50 | |
| D426N | 0.88 | 0.13 | 14.69 | 1.01 | 0.07 | 6.48 | 10.58 | 14.50 | |
| D426P | 0.19 | 0.01 | 6.91 | 0.14 | 0.00 | 0.00 | 3.46 | 14.50 | |
| D426Q | 0.27 | 0.03 | 9.43 | 0.34 | 0.04 | 11.44 | 10.44 | 25.43 | |
| D426S | 0.30 | 0.07 | 22.35 | 0.30 | 0.00 | 0.00 | 11.18 | 25.43 | |
| G427H | 0.48 | 0.18 | 38.15 | 0.40 | 0.13 | 33.63 | 35.89 | 32.10 | |
| V428L | 2.65 | 0.38 | 14.21 | 2.66 | 0.28 | 10.34 | 12.27 | 25.43 | |
| V428P | 0.31 | 0.18 | 59.13 | 0.23 | 0.18 | 78.54 | 68.83 | 21.41 | 119 |
| D431A | 0.40 | 0.22 | 55.21 | 0.24 | 0.20 | 84.18 | 69.69 | 21.41 | 123 |
| D431E | 1.68 | 0.53 | 31.66 | 1.68 | 0.36 | 21.30 | 26.48 | 25.43 | |
| D431G | 0.52 | 0.27 | 51.72 | 0.51 | 0.25 | 49.95 | 50.84 | 21.41 | 362 |
| D431H | 3.69 | 1.27 | 34.37 | 3.12 | 0.89 | 28.43 | 31.40 | 32.10 | |
| D431I | 3.76 | 0.72 | 19.10 | 3.58 | 0.68 | 18.93 | 19.01 | 8.62 | |
| D431K | 6.04 | 1.01 | 16.75 | 5.02 | 0.93 | 18.55 | 17.65 | 8.62 | |
| D431N | 1.76 | 0.21 | 11.85 | 2.85 | 0.33 | 11.63 | 11.74 | 8.62 | |
| D431Q | 2.00 | 0.36 | 17.87 | 2.56 | 0.29 | 11.34 | 14.61 | 8.62 | |
| D431R | 2.80 | 0.98 | 35.12 | 2.65 | 0.87 | 33.04 | 34.08 | 31.17 | |
| D431S | 2.24 | 0.28 | 12.68 | 2.29 | 0.39 | 17.08 | 14.88 | 8.62 | |
| D431V | 1.41 | 0.54 | 38.22 | 1.37 | 0.49 | 36.01 | 37.12 | 31.17 | |
| D431Y | 0.99 | 0.39 | 39.36 | 0.97 | 0.29 | 29.69 | 34.53 | 32.10 | |
| A432E | 0.59 | 0.00 | 0.00 | 0.65 | 0.02 | 2.47 | 1.23 | 8.62 | |
| A432G | 0.50 | 0.01 | 1.01 | 0.49 | 0.00 | 0.00 | 0.50 | 8.62 | |
| A432V | 0.73 | 0.00 | 0.27 | 0.73 | 0.00 | 0.48 | 0.38 | 8.62 | |
| F433A | 0.78 | 0.25 | 32.22 | 0.72 | 0.26 | 36.15 | 34.18 | 13.62 | |
| F433C | 0.94 | 0.29 | 30.75 | 0.67 | 0.26 | 38.58 | 34.66 | 32.10 | |
| F433D | 0.29 | 0.00 | 1.22 | 0.36 | 0.08 | 23.57 | 12.40 | 13.62 | |
| F433H | 0.18 | 0.00 | 0.00 | 0.18 | 0.00 | 0.00 | 0.00 | 13.62 | |
| F433I | 0.16 | 0.00 | 0.00 | 0.21 | 0.00 | 0.00 | 0.00 | 13.62 | |
| F433K | 0.85 | 0.16 | 19.22 | 1.09 | 0.47 | 42.94 | 31.08 | 13.62 | |
| F433L | 0.20 | 0.13 | 65.22 | 0.17 | 0.11 | 62.07 | 63.64 | 35.08 | 331 |
| F433P | 0.41 | 0.01 | 3.33 | 0.58 | 0.17 | 29.80 | 16.57 | 13.62 | |
| F433R | 4.18 | 1.24 | 29.68 | 4.91 | 1.11 | 22.65 | 26.17 | 25.43 | |
| F433S | 2.92 | 0.91 | 31.20 | 3.10 | 0.74 | 23.88 | 27.54 | 25.43 | |
| F433T | 0.24 | 0.17 | 71.70 | 0.15 | 0.14 | 89.18 | 80.44 | 35.08 | 332 |
| F433V | 2.22 | 1.29 | 57.81 | 2.15 | 0.88 | 41.01 | 49.41 | 35.08 | |
| L434F | 1.02 | 0.61 | 60.05 | 0.73 | 0.26 | 36.09 | 48.07 | 35.08 | |
| L434F | 0.95 | 0.33 | 34.32 | 0.88 | 0.30 | 33.43 | 33.87 | 31.17 | |
| L434I | 1.03 | 0.62 | 60.25 | 0.60 | 0.25 | 42.42 | 51.33 | 35.08 | 365 |
| L434M | 2.93 | 2.10 | 71.68 | 1.99 | 0.85 | 42.66 | 57.17 | 35.08 | 366 |
| L434V | 0.75 | 0.28 | 37.15 | 0.83 | 0.35 | 42.04 | 39.59 | 27.64 | |
| K435E | 0.57 | 0.22 | 37.98 | 0.63 | 0.18 | 28.56 | 33.27 | 27.64 | |
| K435G | 0.56 | 0.21 | 36.90 | 0.64 | 0.25 | 38.85 | 37.87 | 27.64 | |
| K435H | 0.13 | 0.06 | 51.60 | 0.15 | 0.09 | 57.48 | 54.54 | 27.64 | 367 |
| K435R | 1.02 | 0.32 | 30.92 | 1.09 | 0.31 | 28.34 | 29.63 | 6.03 | |
| K435S | 0.31 | 0.15 | 48.09 | 0.35 | 0.11 | 31.02 | 39.55 | 27.64 | |
| K435T | 0.51 | 0.15 | 30.53 | 0.51 | 0.13 | 25.12 | 27.83 | 6.03 | |
| K435Y | 0.48 | 0.23 | 47.38 | 0.45 | 0.23 | 51.83 | 49.61 | 37.32 | |
| P436D | 2.09 | 0.57 | 27.09 | 1.64 | 0.39 | 23.78 | 25.43 | 32.10 | |
| P436E | 2.12 | 0.77 | 36.33 | 2.37 | 0.73 | 30.90 | 33.61 | 37.32 | |
| P436G | 0.43 | 0.21 | 48.83 | 0.49 | 0.18 | 37.70 | 43.27 | 37.32 | |
| P436H | 0.60 | 0.25 | 42.24 | 0.64 | 0.24 | 36.75 | 39.49 | 37.32 | |
| P436I | 0.50 | 0.19 | 38.46 | 0.53 | 0.17 | 31.16 | 34.81 | 23.27 | |
| P436K | 0.85 | 0.24 | 27.95 | 0.96 | 0.32 | 33.77 | 30.86 | 37.32 | |
| P436L | 0.17 | 0.08 | 44.31 | 0.22 | 0.07 | 32.87 | 38.59 | 37.32 | |
| P436M | 1.21 | 0.18 | 15.21 | 1.09 | 0.12 | 10.63 | 12.92 | 6.03 | |
| P436Q | 1.98 | 0.63 | 31.86 | 1.66 | 0.55 | 33.37 | 32.62 | 29.87 | |
| P436R | 2.25 | 0.61 | 27.32 | 2.29 | 0.73 | 31.94 | 29.63 | 32.10 | |
| P436S | 0.85 | 0.22 | 26.18 | 0.91 | 0.19 | 21.37 | 23.78 | 6.03 | |
| P436T | 0.50 | 0.11 | 22.27 | 0.52 | 0.15 | 29.90 | 26.08 | 29.87 | |
| P436W | 0.24 | 0.00 | 0.00 | 0.28 | 0.01 | 2.15 | 1.08 | 33.76 | |
| P436Y | 0.58 | 0.17 | 29.77 | 0.55 | 0.10 | 18.96 | 24.36 | 6.03 | |
| P437A | 0.19 | 0.09 | 49.33 | 0.23 | 0.05 | 22.81 | 36.07 | 29.87 | |
| P437D | 1.11 | 0.36 | 32.85 | 1.14 | 0.29 | 25.14 | 29.00 | 29.87 | |
| P437H | 1.04 | 0.30 | 28.70 | 0.68 | 0.21 | 31.11 | 29.91 | 31.22 | |
| P437I | 0.54 | 0.16 | 28.86 | 0.54 | 0.10 | 17.85 | 23.36 | 6.03 | |
| P437K | 1.81 | 0.30 | 16.64 | 1.35 | 0.34 | 25.30 | 20.97 | 31.22 | |
| P437L | 0.80 | 0.16 | 19.43 | 0.71 | 0.15 | 21.14 | 20.28 | 31.22 | |
| P437M | 0.32 | 0.06 | 19.50 | 0.34 | 0.08 | 22.44 | 20.97 | 31.22 | |
| P437Q | 1.59 | 0.39 | 24.21 | 1.33 | 0.36 | 26.73 | 25.47 | 31.22 | |
| P437R | 0.74 | 0.25 | 33.95 | 0.90 | 0.28 | 31.21 | 32.58 | 23.27 | |
| P437S | 1.08 | 0.32 | 29.55 | 1.26 | 0.29 | 22.88 | 26.22 | 6.03 | |
| M438A | 0.32 | 0.16 | 50.54 | 0.35 | 0.58 | 165.29 | 107.91 | 30.85 | 170 |
| M438C | 0.99 | 0.35 | 35.67 | 1.82 | 1.07 | 58.77 | 47.22 | 30.85 | |
| M438D | 1.44 | 0.45 | 31.02 | 1.14 | 0.35 | 30.89 | 30.96 | 4.17 | |
| M438E | 2.06 | 0.54 | 26.22 | 3.89 | 1.61 | 41.43 | 33.83 | 30.85 | |
| M438L | 2.08 | 0.76 | 36.66 | 3.24 | 1.26 | 39.00 | 37.83 | 30.85 | |
| M438N | 1.83 | 0.55 | 30.11 | 4.21 | 1.88 | 44.78 | 37.45 | 30.85 | |
| M438P | 4.80 | 1.88 | 39.25 | 3.20 | 0.91 | 28.34 | 33.80 | 28.95 | |
| M438Q | 2.60 | 0.85 | 32.63 | 2.43 | 0.74 | 30.32 | 31.47 | 28.95 | |
| M438R | 2.84 | 1.00 | 35.07 | 2.85 | 0.76 | 26.55 | 30.81 | 28.95 | |
| M438S | 2.73 | 0.87 | 32.07 | 2.59 | 0.92 | 35.43 | 33.75 | 28.95 | |
| M438T | 1.78 | 0.90 | 50.32 | 1.56 | 0.54 | 34.45 | 42.39 | 28.95 | |
| M438V | 0.35 | 0.18 | 49.86 | 0.41 | 0.17 | 41.28 | 45.57 | 24.69 | |
| M438W | 1.05 | 0.38 | 36.45 | 0.73 | 0.26 | 35.79 | 36.12 | 28.95 | |
| E439A | 1.89 | 0.64 | 33.70 | 1.44 | 0.48 | 33.06 | 33.38 | 28.95 | |
| E439C | 0.13 | 0.10 | 79.61 | 0.13 | 0.08 | 58.17 | 68.89 | 28.95 | 328 |
| E439F | 1.60 | 0.32 | 20.09 | 1.28 | 0.45 | 35.34 | 27.72 | 28.51 | |
| E439G | 1.35 | 0.44 | 32.26 | 1.31 | 0.34 | 25.57 | 28.92 | 28.51 | |
| E439H | 2.20 | 0.73 | 32.98 | 1.72 | 0.53 | 30.87 | 31.92 | 28.51 | |
| E439K | 0.48 | 0.22 | 46.48 | 0.50 | 0.26 | 52.55 | 49.51 | 28.51 | |
| E439L | 0.41 | 0.11 | 27.28 | 0.41 | 0.11 | 26.50 | 26.89 | 37.72 | |
| E439P | 1.48 | 0.46 | 31.31 | 1.16 | 0.26 | 22.27 | 26.79 | 28.51 | |
| E439Q | 2.34 | 0.45 | 19.31 | 1.77 | 0.51 | 29.13 | 24.22 | 28.51 | |
| E439S | 0.76 | 0.35 | 46.83 | 0.60 | 0.14 | 23.64 | 35.24 | 28.51 | |
| E439T | 1.23 | 0.58 | 46.88 | 1.16 | 0.77 | 66.25 | 56.57 | 35.74 | 370 |
| E439V | 1.30 | 0.61 | 47.15 | 1.19 | 0.44 | 37.06 | 42.11 | 35.74 | |
| E439W | 0.88 | 0.39 | 44.17 | 0.62 | 0.28 | 45.79 | 44.98 | 35.74 | |
| T440A | 1.59 | 0.61 | 38.22 | 0.97 | 0.44 | 45.19 | 41.71 | 35.74 | |
| T440D | 0.69 | 0.35 | 50.43 | 0.80 | 0.36 | 45.63 | 48.03 | 35.74 | |
| T440E | 0.68 | 0.21 | 30.75 | 0.75 | 0.20 | 27.15 | 28.95 | 23.27 | |
| T440F | 1.33 | 0.54 | 40.81 | 1.00 | 0.38 | 38.37 | 39.59 | 35.74 | |
| T440H | 0.83 | 0.26 | 31.52 | 0.97 | 0.26 | 27.24 | 29.38 | 23.27 | |
| T440I | 0.58 | 0.10 | 16.57 | 0.63 | 0.09 | 14.91 | 15.74 | 4.17 | |
| T440L | 1.35 | 0.28 | 20.86 | 1.18 | 0.41 | 34.56 | 27.71 | 35.74 | |
| T440M | 2.26 | 1.44 | 63.52 | 1.79 | 0.84 | 46.66 | 55.09 | 24.48 | 371 |
| T440P | 1.55 | 0.55 | 35.32 | 1.39 | 0.51 | 37.11 | 36.22 | 24.48 | |
| T440R | 1.11 | 0.38 | 33.69 | 1.15 | 0.40 | 34.72 | 34.21 | 24.48 | |
| T440S | 0.96 | 0.30 | 31.17 | 0.78 | 0.20 | 25.54 | 28.36 | 24.48 | |
| T440V | 0.63 | 0.19 | 30.02 | 0.84 | 0.23 | 27.83 | 28.92 | 23.27 | |
| T440Y | 0.98 | 0.08 | 7.63 | 0.92 | 0.29 | 30.95 | 19.29 | 24.48 | |
| E441A | 0.55 | 0.21 | 37.74 | 0.46 | 0.21 | 44.13 | 40.94 | 24.48 | |
| E441D | 1.17 | 0.35 | 29.59 | 0.92 | 0.29 | 31.40 | 30.50 | 24.48 | |
| E441F | 1.29 | 0.41 | 31.63 | 0.90 | 0.31 | 33.87 | 32.75 | 30.59 | |
| E441G | 0.47 | 0.02 | 4.06 | 0.48 | 0.09 | 17.92 | 10.99 | 33.76 | |
| E441H | 1.35 | 0.34 | 24.82 | 1.16 | 0.34 | 29.10 | 26.96 | 30.59 | |
| E441K | 0.98 | 0.26 | 26.77 | 0.76 | 0.26 | 34.56 | 30.66 | 30.59 | |
| E441L | 1.44 | 0.40 | 28.04 | 1.19 | 0.28 | 23.60 | 25.82 | 30.59 | |
| E441N | 2.35 | 0.53 | 22.61 | 1.64 | 0.64 | 39.21 | 30.91 | 30.59 | |
| E441S | 2.59 | 0.57 | 21.91 | 2.04 | 0.55 | 26.95 | 24.43 | 30.59 | |
| E441T | 2.33 | 0.70 | 30.18 | 2.26 | 3.17 | 140.00 | 85.09 | 18.62 | 176 |
| E441V | 0.33 | 0.21 | 61.73 | 0.54 | 0.29 | 52.58 | 57.16 | 18.62 | 373 |
| E441Y | 1.34 | 0.53 | 39.80 | 1.87 | 0.78 | 41.60 | 40.70 | 18.62 | |
| E442G | 0.91 | 0.36 | 39.55 | 1.16 | 0.56 | 47.72 | 43.64 | 18.62 | |
| E442K | 1.69 | 0.58 | 34.23 | 2.77 | 1.02 | 36.80 | 35.52 | 18.62 | |
| E442L | 1.20 | 0.40 | 33.36 | 1.47 | 0.11 | 7.12 | 20.24 | 18.62 | |
| E442P | 1.26 | 0.75 | 59.06 | 1.29 | 0.61 | 46.85 | 52.95 | 29.77 | 374 |
| E442Q | 1.40 | 0.68 | 48.43 | 1.34 | 0.46 | 34.16 | 41.29 | 29.77 | |
| E442R | 1.09 | 0.47 | 42.76 | 1.07 | 0.40 | 37.23 | 39.99 | 29.77 | |
| E442T | 0.54 | 0.25 | 46.31 | 0.46 | 0.20 | 43.37 | 44.84 | 29.77 | |
| E442V | 0.85 | 0.38 | 44.44 | 0.68 | 0.28 | 41.38 | 42.91 | 29.77 | |
| E442W | 0.34 | 0.12 | 36.30 | 0.40 | 0.13 | 32.21 | 34.25 | 23.27 | |
| E442Y | 0.50 | 0.24 | 48.14 | 0.38 | 0.17 | 45.38 | 46.76 | 29.77 | |
| P443A | 1.59 | 0.68 | 42.99 | 1.16 | 0.41 | 34.94 | 38.97 | 29.77 | |
| P443E | 1.73 | 0.84 | 48.34 | 1.10 | 0.43 | 38.74 | 43.54 | 29.77 | |
| P443F | 1.01 | 0.42 | 41.32 | 1.04 | 0.33 | 32.08 | 36.70 | 25.76 | |
| P443G | 1.45 | 0.50 | 34.29 | 1.39 | 0.43 | 30.80 | 32.54 | 25.76 | |
| P443H | 1.57 | 0.51 | 32.49 | 1.54 | 0.43 | 28.18 | 30.34 | 25.76 | |
| P443L | 0.45 | 0.03 | 7.51 | 0.47 | 0.04 | 8.14 | 7.83 | 33.76 | |
| P443M | 0.15 | 0.13 | 87.07 | 0.13 | 0.10 | 80.48 | 83.78 | 38.36 | 140 |
| P443N | 0.74 | 0.28 | 37.29 | 1.56 | 0.25 | 16.27 | 26.78 | 25.76 | |
| P443Q | 1.42 | 0.46 | 32.19 | 1.48 | 0.43 | 29.05 | 30.62 | 25.76 | |
| P443R | 1.06 | 0.35 | 33.11 | 1.13 | 0.32 | 28.39 | 30.75 | 25.76 | |
| P443S | 1.89 | 0.59 | 31.25 | 1.69 | 0.42 | 25.16 | 28.20 | 25.79 | |
| P443T | 1.46 | 0.42 | 28.50 | 1.51 | 0.34 | 22.77 | 25.63 | 25.79 | |
| P443W | 0.26 | 0.11 | 41.09 | 0.26 | 0.08 | 31.74 | 36.41 | 25.79 | |
| Q444E | 2.98 | 0.59 | 19.93 | 3.41 | 0.62 | 18.16 | 19.04 | 27.07 | |
| Q444F | 0.82 | 0.17 | 20.69 | 0.66 | 0.13 | 20.27 | 20.48 | 27.07 | |
| Q444G | 1.49 | 0.31 | 20.92 | 1.34 | 0.27 | 20.49 | 20.71 | 27.07 | |
| Q444H | 3.65 | 0.51 | 14.06 | 2.56 | 0.55 | 21.33 | 17.69 | 27.07 | |
| Q444I | 3.19 | 0.87 | 27.21 | 4.04 | 1.07 | 26.47 | 26.84 | 27.07 | |
| Q444K | 3.58 | 1.64 | 45.79 | 6.70 | 3.09 | 46.07 | 45.93 | 31.60 | |
| Q444N | 2.39 | 0.59 | 24.67 | 2.35 | 0.57 | 24.48 | 24.58 | 31.60 | |
| Q444R | 1.86 | 0.55 | 29.40 | 1.96 | 0.42 | 21.30 | 25.35 | 31.60 | |
| Q444W | 1.17 | 0.32 | 27.09 | 1.10 | 0.28 | 25.85 | 26.47 | 31.60 | |
| Q444Y | 0.87 | 0.22 | 24.73 | 0.71 | 0.20 | 28.24 | 26.48 | 31.60 | |
| I445A | 0.77 | 0.22 | 29.00 | 0.65 | 0.15 | 23.14 | 26.07 | 25.79 | |
| I445G | 2.53 | 0.58 | 22.83 | 2.24 | 0.45 | 20.05 | 21.44 | 25.79 | |
| I445L | 1.36 | 0.35 | 25.64 | 0.99 | 0.23 | 23.30 | 24.47 | 25.79 | |
| I445M | 2.18 | 0.52 | 23.98 | 2.12 | 0.37 | 17.47 | 20.72 | 25.79 | |
| I445N | 3.78 | 1.18 | 31.31 | 3.52 | 0.70 | 20.00 | 25.65 | 25.79 | |
| I445P | 2.64 | 0.64 | 24.15 | 2.30 | 0.68 | 29.69 | 26.92 | 29.86 | |
| I445Q | 2.24 | 0.58 | 25.77 | 2.08 | 0.49 | 23.38 | 24.57 | 29.86 | |
| I445R | 2.33 | 0.54 | 23.00 | 2.05 | 0.47 | 22.98 | 22.99 | 29.86 | |
| I445S | 2.79 | 0.65 | 23.47 | 2.17 | 0.48 | 22.04 | 22.76 | 29.86 | |
| I445T | 1.99 | 0.46 | 22.84 | 1.98 | 0.49 | 25.02 | 23.93 | 29.86 | |
| I445V | 1.92 | 0.43 | 22.22 | 2.03 | 0.43 | 21.00 | 21.61 | 29.86 | |
| I445W | 1.25 | 0.30 | 24.05 | 1.36 | 0.30 | 21.98 | 23.02 | 29.86 | |
| I445Y | 0.22 | 0.40 | 181.42 | 0.30 | 0.38 | 126.09 | 153.75 | 38.05 | 111 |
| F446A | 1.08 | 0.36 | 33.26 | 1.25 | 0.40 | 31.64 | 32.45 | 23.27 | |
| F446C | 0.17 | 0.16 | 92.20 | 0.19 | 0.16 | 85.60 | 88.90 | 36.18 | 235 |
| F446D | 1.58 | 0.93 | 58.67 | 1.56 | 1.07 | 68.78 | 63.72 | 36.18 | 289 |
| F446E | 0.39 | 0.22 | 56.56 | 0.36 | 0.26 | 70.47 | 63.51 | 36.18 | 238 |
| F446G | 0.22 | 0.13 | 61.47 | 0.25 | 0.16 | 62.30 | 61.88 | 36.18 | 239 |
| F446H | 2.29 | 0.79 | 34.59 | 1.80 | 0.61 | 34.00 | 34.29 | 26.04 | |
| F446I | 1.81 | 0.62 | 34.38 | 1.38 | 0.37 | 26.76 | 30.57 | 26.04 | |
| F446K | 1.80 | 0.62 | 34.27 | 1.78 | 0.49 | 27.59 | 30.93 | 26.04 | |
| F446L | 1.37 | 0.52 | 37.96 | 1.50 | 0.41 | 27.30 | 32.63 | 26.04 | |
| F446M | 1.76 | 0.88 | 50.16 | 1.49 | 0.57 | 38.51 | 44.33 | 26.04 | |
| F446Q | 1.66 | 0.60 | 36.36 | 1.69 | 0.50 | 29.79 | 33.07 | 26.04 | |
| F446R | 1.20 | 0.44 | 36.77 | 1.33 | 0.42 | 31.46 | 34.12 | 26.04 | |
| F446T | 2.32 | 0.94 | 40.32 | 2.55 | 0.62 | 24.14 | 32.23 | 26.04 | |
| F446V | 2.36 | 0.77 | 32.46 | 2.08 | 0.83 | 40.12 | 36.29 | 22.16 | |
| F446W | 1.31 | 0.51 | 38.84 | 1.43 | 0.72 | 50.09 | 44.46 | 23.27 | |
| Y447D | 0.55 | 0.30 | 54.28 | 0.51 | 0.29 | 56.95 | 55.61 | 21.73 | 381 |
| Y447E | 1.72 | 1.41 | 82.08 | 1.67 | 1.43 | 85.36 | 83.72 | 66.14 | 185 |
| Y447F | 1.20 | 0.60 | 50.00 | 1.38 | 0.68 | 49.20 | 49.60 | 25.82 | |
| Y447G | 0.46 | 0.79 | 172.56 | 0.50 | 0.74 | 147.11 | 159.83 | 66.14 | 86 |
| Y447I | 1.51 | 0.38 | 25.26 | 1.48 | 0.45 | 30.35 | 27.80 | 16.94 | |
| Y447L | 0.94 | 0.26 | 27.83 | 0.79 | 0.31 | 39.04 | 33.44 | 4.69 | |
| Y447M | 0.86 | 0.24 | 27.21 | 0.77 | 0.18 | 24.09 | 25.65 | 4.69 | |
| Y447N | 1.68 | 0.50 | 29.90 | 1.59 | 0.50 | 31.20 | 30.55 | 17.50 | |
| Y447P | 0.38 | 0.13 | 35.77 | 0.35 | 0.11 | 31.32 | 33.55 | 17.50 | |
| Y447Q | 1.20 | 0.37 | 31.16 | 1.29 | 0.36 | 28.02 | 29.59 | 17.50 | |
| Y447R | 0.93 | 0.30 | 32.47 | 1.04 | 0.27 | 25.74 | 29.11 | 17.50 | |
| Y447T | 0.95 | 0.34 | 35.93 | 0.93 | 0.33 | 35.63 | 35.78 | 17.50 | |
| Y447V | 0.90 | 0.29 | 31.76 | 0.89 | 0.31 | 35.00 | 33.38 | 17.50 | |

2. Confirmation Screening

A confirmation screening was completed for 176 selected variants. The confirmation screen was performed as described above. The results are set forth in Table 11. The Table below sets forth the average OD for all tested transfection and duplicates of each sample at 4° C. and 52° C., and the average percent activity remaining at 52° C. compared to 4° C. (% Act. 52° C. vs. 4° C.). For comparison, the Table also sets forth the percent activity of wildtype (unmodified) PH20 control at 52° C. compared to 4° C. as tested in the same assay plate.

In the confirmation screening assay, the average PH20 activity of the tested variants at 4° C. was substantially lower for some of the tested variants than it was in the primary screening assay. This difference likely reflects lower expression of the transfected variants in the supernatants tested during the confirmation screening. For example, the variant N369H had an average activity normalized to SEAP expression of about 3.50 U/mL in the primary screen, but negligible to no activity in the confirmation screen. Other variants appeared to have expressed normally as evidenced by similar activities at 4° C. in the primary screen and confirmation screen.

TABLE 11
Confirmation Screen

| | | | Average | |
|---|---|---|---|---|
| | | | % Act. | % Act. wt |
| | Avg. Act. | Avg. Act. | 52° C. vs. | 52° C. vs. |
| Variant | 4° C. | 52° C. | 4° C. | 4° C. |
| N011G | 7.28 | 2.04 | 28.00 | 35.75 |
| L015V | 1.05 | 0.35 | 33.52 | 10.50 |
| L026S | 0.39 | 0.12 | 31.75 | 29.20 |
| G027E | 1.05 | 0.34 | 32.09 | 23.68 |
| G027H | 0.28 | 0.16 | 56.24 | 10.50 |
| F029E | 5.30 | 0.87 | 16.50 | 39.22 |
| V206I | 0.12 | 0.18 | 148.79 | 37.44 |
| F029S | 1.19 | 0.37 | 31.34 | 23.68 |
| D030A | 1.68 | 0.38 | 22.82 | 34.04 |
| S261A | 1.40 | 1.82 | 130.17 | 25.82 |
| E031G | 0.82 | 0.23 | 27.66 | 23.68 |
| E031L | 2.68 | 0.55 | 20.48 | 26.20 |
| P032Q | 0.74 | 0.21 | 27.63 | 10.50 |
| P032W | 0.24 | 0.12 | 49.90 | 17.47 |
| L033G | 0.36 | 0.05 | 14.89 | 31.45 |
| L033M | 0.31 | 0.14 | 46.35 | 17.47 |
| L033R | 0.76 | 0.23 | 30.43 | 34.04 |
| L033W | 0.69 | 0.17 | 24.96 | 31.45 |
| D034H | 0.46 | 0.10 | 21.79 | 31.45 |
| D034W | 0.19 | 0.10 | 52.51 | 39.22 |
| D284A | 0.13 | 0.15 | 116.73 | 37.44 |
| F038Y | 0.66 | 0.16 | 24.28 | 23.68 |
| S039R | 0.29 | 0.09 | 32.19 | 39.22 |
| S039T | 0.95 | 0.30 | 32.22 | 23.68 |
| I041T | 1.39 | 0.42 | 30.16 | 34.04 |
| I041W | 1.20 | 0.29 | 24.20 | 23.68 |
| F204P | 0.82 | 0.89 | 107.90 | 29.80 |
| G050D | 0.57 | 0.19 | 32.72 | 23.68 |
| G052N | 4.25 | 1.51 | 35.60 | 35.75 |
| G052T | 1.14 | 0.29 | 25.77 | 26.34 |
| T054N | 0.48 | 0.12 | 25.08 | 37.68 |
| V058K | 14.55 | 7.37 | 50.68 | 35.75 |
| Q143K | 0.22 | 0.22 | 100.23 | 37.44 |
| S315T | 0.21 | 0.20 | 98.79 | 26.20 |
| I067F | 0.61 | 0.28 | 45.25 | 37.44 |
| S069A | 1.10 | 0.32 | 29.20 | 17.47 |
| S069E | 0.71 | 0.22 | 30.67 | 37.68 |
| S069M | 1.47 | 0.58 | 39.78 | 35.75 |
| S069P | 1.00 | 0.39 | 38.96 | 37.68 |
| S069T | 0.41 | 0.18 | 44.17 | 23.68 |
| S069Y | 0.97 | 0.18 | 18.80 | 26.34 |
| I070A | 2.75 | 0.90 | 32.89 | 35.75 |
| I070C | 0.49 | 0.17 | 34.66 | 37.68 |
| V073H | 3.72 | 1.18 | 31.86 | 35.75 |
| V073Q | 2.03 | 0.82 | 40.49 | 37.68 |
| V073T | 0.80 | 0.27 | 33.27 | 26.34 |
| T074C | 0.45 | 0.16 | 35.29 | 37.68 |
| I083Q | 1.08 | 0.31 | 29.08 | 25.38 |
| S084D | 0.45 | 0.21 | 46.54 | 17.47 |
| Q086D | 0.72 | 0.32 | 44.39 | 29.80 |
| Q086R | 0.34 | 0.12 | 36.27 | 31.45 |
| D087P | 0.82 | 0.21 | 25.53 | 37.44 |
| D090W | 1.12 | 0.21 | 18.73 | 31.45 |
| A092V | 0.45 | 0.14 | 31.35 | 10.50 |
| K093E | 0.80 | 0.29 | 36.90 | 26.20 |
| K093S | 0.30 | 0.11 | 38.64 | 17.47 |
| T097F | 1.29 | 0.30 | 23.35 | 34.04 |
| F098M | 0.59 | 0.28 | 47.80 | 29.80 |
| V102H | 1.18 | 0.62 | 52.72 | 10.50 |
| N131C | 0.29 | 0.09 | 31.70 | 26.20 |
| N131L | 1.18 | 0.27 | 23.29 | 34.04 |
| Q138W | 0.33 | 0.09 | 26.77 | 25.38 |
| Q139V | 0.41 | 0.11 | 27.68 | 39.22 |
| N141M | 0.79 | 0.29 | 36.08 | 10.50 |
| N141Y | 0.33 | 0.18 | 53.86 | 26.20 |
| S215T | 0.26 | 0.25 | 96.58 | 35.75 |
| L144G | 0.17 | 0.09 | 53.87 | 39.22 |
| S235T | 0.21 | 0.17 | 79.02 | 26.20 |
| K152I | 2.40 | 0.55 | 22.79 | 39.22 |
| K152M | 0.52 | 0.19 | 35.93 | 23.68 |
| K154R | 2.36 | 0.57 | 24.16 | 22.21 |
| Q155A | 1.26 | 0.36 | 28.75 | 30.11 |
| Q155D | 0.32 | 0.17 | 53.99 | 17.47 |
| Q155F | 3.13 | 0.71 | 22.62 | 22.21 |
| Q155H | 0.56 | 0.19 | 33.63 | 10.50 |
| Q155L | 1.33 | 0.37 | 27.84 | 30.11 |
| Q155R | 2.13 | 0.49 | 22.83 | 30.11 |
| Q155S | 0.71 | 0.19 | 26.83 | 30.11 |
| K159A | 0.79 | 0.16 | 19.87 | 30.11 |
| K159H | 1.11 | 0.28 | 25.06 | 30.11 |
| F204P | 2.25 | 1.73 | 76.77 | 26.34 |
| K159Q | 0.45 | 0.16 | 34.68 | 29.80 |
| D163R | 0.39 | 0.19 | 49.03 | 22.21 |
| D163S | 1.18 | 0.30 | 25.15 | 23.68 |
| L165F | 1.16 | 0.59 | 50.62 | 29.80 |
| L174W | 0.21 | 0.12 | 56.63 | 39.22 |
| K195H | 0.92 | 0.39 | 41.93 | 29.20 |
| K195L | 0.68 | 0.27 | 39.79 | 29.20 |
| P197F | 0.41 | 0.10 | 24.97 | 29.20 |
| G198L | 0.31 | 0.15 | 48.80 | 10.50 |
| T147I | 0.28 | 0.20 | 73.68 | 29.80 |
| T306D | 0.32 | 0.24 | 73.42 | 29.80 |
| N205A | 0.26 | 0.15 | 57.01 | 17.47 |
| N205E | 2.55 | 1.07 | 42.03 | 35.75 |
| N205K | 0.82 | 0.20 | 25.00 | 17.47 |
| N205L | 0.58 | 0.27 | 47.23 | 17.47 |
| N205P | 2.35 | 1.05 | 44.82 | 35.75 |
| N205S | 1.57 | 0.62 | 39.63 | 37.68 |
| N205T | 1.05 | 0.38 | 36.30 | 37.68 |
| N369H | 0.26 | 0.19 | 72.15 | 29.80 |
| I208L | 1.29 | 0.33 | 25.72 | 37.68 |
| I208Q | 3.36 | 0.83 | 24.80 | 35.75 |
| I208R | 1.03 | 0.25 | 24.29 | 17.47 |
| D213E | 0.40 | 0.24 | 58.61 | 37.44 |
| D213N | 1.63 | 0.63 | 38.67 | 17.47 |
| S215A | 2.88 | 0.75 | 25.88 | 26.34 |
| S215D | 2.87 | 0.62 | 21.52 | 35.75 |
| S215E | 1.92 | 0.47 | 24.50 | 26.34 |
| S215H | 1.42 | 0.31 | 21.46 | 26.34 |
| S215L | 2.06 | 0.32 | 15.46 | 26.34 |
| S215Q | 1.16 | 0.23 | 19.96 | 26.34 |
| F029H | 0.31 | 0.21 | 69.98 | 26.20 |
| S215V | 4.77 | 0.98 | 20.56 | 35.75 |
| S215W | 1.80 | 0.35 | 19.39 | 26.34 |
| N219A | 2.33 | 0.37 | 15.87 | 26.34 |
| N219I | 1.47 | 0.34 | 22.99 | 35.75 |
| N219K | 5.75 | 1.64 | 28.52 | 35.75 |
| N219R | 1.49 | 0.36 | 24.54 | 37.68 |
| N219S | 2.38 | 0.51 | 21.53 | 37.68 |
| N219T | 1.45 | 0.26 | 17.65 | 37.68 |
| E220V | 6.88 | 3.92 | 57.06 | 35.75 |
| T222N | 0.21 | 0.08 | 37.71 | 29.20 |
| Q234M | 0.55 | 0.16 | 29.85 | 37.68 |
| R311G | 0.30 | 0.21 | 69.87 | 37.44 |
| V237Q | 3.00 | 0.78 | 26.12 | 22.21 |
| T240Q | 0.70 | 0.27 | 39.11 | 29.20 |
| I251L | 1.15 | 0.42 | 36.34 | 34.04 |
| I251M | 0.47 | 0.18 | 37.24 | 34.04 |
| A259K | 0.97 | 0.22 | 23.07 | 34.04 |
| A259P | 1.69 | 0.56 | 33.24 | 23.68 |
| A048G | 0.12 | 0.08 | 69.14 | 39.22 |
| R060K | 0.88 | 0.58 | 65.78 | 37.68 |
| L263T | 0.44 | 0.05 | 11.71 | 31.45 |
| V277A | 0.30 | 0.17 | 57.53 | 10.50 |
| V277C | 0.23 | 0.12 | 53.20 | 29.20 |
| L278N | 3.68 | 1.12 | 30.50 | 10.50 |
| S261F | 0.27 | 0.17 | 63.79 | 37.44 |
| D284Q | 1.25 | 0.38 | 30.24 | 34.04 |
| D284S | 0.32 | 0.16 | 50.31 | 22.21 |
| E285M | 0.38 | 0.05 | 12.27 | 29.20 |
| E292V | 1.10 | 0.31 | 27.77 | 31.45 |
| G305N | 0.45 | 0.16 | 35.35 | 29.20 |
| V058R | 3.03 | 1.91 | 63.06 | 35.75 |
| M310A | 5.89 | 1.49 | 25.34 | 26.34 |
| M310Q | 3.09 | 1.46 | 47.42 | 37.68 |
| K159N | 0.45 | 0.28 | 62.67 | 29.80 |
| A412S | 0.17 | 0.11 | 62.36 | 10.50 |
| L317N | 1.99 | 0.57 | 28.49 | 22.21 |
| S342A | 1.67 | 0.37 | 22.12 | 39.22 |
| D368H | 0.30 | 0.02 | 5.19 | 30.11 |
| D030P | 0.31 | 0.19 | 61.80 | 23.68 |
| G377T | 2.66 | 0.65 | 24.43 | 37.44 |
| K379H | 0.50 | 0.22 | 44.48 | 29.80 |
| K379S | 0.22 | 0.00 | 0.00 | 39.22 |
| K379T | 0.36 | 0.13 | 35.05 | 29.80 |
| F380I | 0.64 | 0.17 | 26.31 | 39.22 |
| F380L | 0.49 | 0.25 | 51.08 | 37.44 |
| F380P | 0.42 | 0.20 | 47.78 | 29.80 |
| S401G | 1.67 | 0.58 | 34.50 | 37.68 |
| L406N | 0.54 | 0.26 | 48.24 | 37.44 |
| S407F | 0.23 | 0.04 | 18.12 | 25.38 |
| E410S | 1.08 | 0.33 | 30.44 | 34.04 |
| A412P | 1.40 | 0.34 | 24.04 | 22.21 |
| L037K | 0.22 | 0.13 | 60.05 | 34.04 |
| V428P | 0.58 | 0.19 | 32.09 | 17.47 |
| D431A | 0.91 | 0.31 | 34.50 | 10.50 |
| F433T | 2.56 | 0.68 | 26.73 | 25.38 |
| M438A | 0.59 | 0.24 | 40.75 | 10.50 |
| E441T | 1.23 | 0.32 | 26.10 | 34.04 |
| I445Y | 0.85 | 0.31 | 37.03 | 31.45 |
| F446C | 0.95 | 0.30 | 31.78 | 26.20 |
| F446D | 2.68 | 0.83 | 31.08 | 34.04 |
| F446E | 1.08 | 0.22 | 20.33 | 39.22 |
| F446G | 0.76 | 0.16 | 20.41 | 23.68 |
| Y447E | 0.94 | 0.44 | 46.49 | 29.80 |
| Y447G | 0.62 | 0.08 | 12.29 | 30.11 |

Since modifications will be apparent to those of skill in the art, it is intended that this invention be limited only by the scope of the appended claims.

1. A modified PH20 polypeptide that is an uber-thermophile exhibiting thermal stability, wherein:
the modified PH20 polypeptide comprises an amino acid replacement in an unmodified PH20 polypeptide, whereby the polypeptide retains at least 50% of its hyaluronidase activity at neutral pH after incubation at 52° C. for 10 minutes compared to its hyaluronidase activity after incubation at 4° C. for 10 minutes; and
the unmodified PH20 polypeptide consists of the sequence of amino acids set forth in SEQ ID NO: 7 or is a C-terminal truncated fragment thereof that is a soluble PH20 polypeptide or a sequence of amino acids that has at least 85% sequence identity to SEQ ID NO:7 or a C-terminal truncated fragment thereof that is soluble.

2. The modified PH20 polypeptide of claim 1, comprising at least one amino acid replacement at an amino acid position corresponding to a position selected from among 10, 11, 13, 15, 26, 27, 28, 29, 30, 31, 32, 33, 34, 36, 37, 38, 39, 41, 46, 47, 48, 49, 50, 58, 60, 67, 69, 72, 73, 83, 84, 86, 87, 90, 92, 93, 94, 97, 98, 99, 102, 105, 114, 118, 120, 131, 132, 135, 138, 139, 141, 142, 143, 144, 146, 147, 148, 150, 151, 152, 154, 155, 156, 158, 159, 160, 161, 162, 163, 165, 170, 174, 195, 196, 197, 198, 202, 204, 205, 206, 208, 213, 215, 219, 220, 222, 234, 235, 237, 240, 247, 251, 255, 259, 260, 261, 263, 265, 271, 276, 277, 278, 282, 284, 285, 290, 292, 305, 306, 309, 310, 311, 315, 317, 318, 320, 321, 328, 342, 343, 349, 359, 368, 369, 371, 373, 374, 375, 376, 377, 379, 380, 388, 389, 393, 399, 401, 403, 406, 407, 410, 412, 413, 415, 417, 419, 421, 428, 431, 433, 434, 435, 438, 439, 440, 441, 442, 443, 445, 446 and 447, with reference to amino acid positions of the sequence set forth in SEQ ID NO:3, wherein corresponding amino acid positions are identified by alignment of the PH20 polypeptide with the polypeptide set forth in SEQ ID NO:3.

3. The modified PH20 polypeptide of claim 2, wherein the replacement is an amino acid replacement selected from among:
at a position corresponding to position 10, replacement with G or N;
at a position corresponding to position 11, replacement with G;
at a position corresponding to position 13, replacement with H;
at a position corresponding to position 15, replacement with A or V;
at a position corresponding to position 26, replacement with P, R, S, V, W or Y;
at a position corresponding to position 27, replacement with E or H;
at a position corresponding to position 28, replacement with L;
at a position corresponding to position 29, replacement with E, H, L, S or W;
at a position corresponding to position 30, replacement with A, P or R;
at a position corresponding to position 31, replacement with C, G or L;
at a position corresponding to position 32, replacement with Q, S, V or W;
at a position corresponding to position 33, replacement with G, M, R or W;
at a position corresponding to position 34, replacement with E, H or W;
at a position corresponding to position 36, replacement with G;
at a position corresponding to position 37, replacement with I or K;
at a position corresponding to position 38, replacement with Y;
at a position corresponding to position 39, replacement with Q, R or T;
at a position corresponding to position 41, replacement with D, T or W;
at a position corresponding to position 46, replacement with H;
at a position corresponding to position 47, replacement with G or R;
at a position corresponding to position 48, replacement with G or Y;
at a position corresponding to position 49, replacement with I;
at a position corresponding to position 50, replacement with C or D;
at a position corresponding to position 58, replacement with K or R;
at a position corresponding to position 60, replacement with K;
at a position corresponding to position 67, replacement with F;
at a position corresponding to position 69, replacement with A or Y;
at a position corresponding to position 72, replacement with D;
at a position corresponding to position 73, replacement with T;
at a position corresponding to position 83, replacement with G, Q or V;
at a position corresponding to position 84, replacement with D;
at a position corresponding to position 86, replacement with D, E, N or R;
at a position corresponding to position 87, replacement with M, P or V;
at a position corresponding to position 90, replacement with E, T or W;
at a position corresponding to position 92, replacement with V;
at a position corresponding to position 93, replacement with E or S;
at a position corresponding to position 94, replacement with N;
at a position corresponding to position 97, replacement with E or F;
at a position corresponding to position 98, replacement with M;
at a position corresponding to position 99, replacement with S;
at a position corresponding to position 102, replacement with H or N;
at a position corresponding to position 105, replacement with I, R or W;
at a position corresponding to position 114, replacement with G;
at a position corresponding to position 118, replacement with M;
at a position corresponding to position 120, replacement with S;
at a position corresponding to position 131, replacement with C or L;
at a position corresponding to position 132, replacement with A or C;
at a position corresponding to position 135, replacement with Q;
at a position corresponding to position 138, replacement with W;
at a position corresponding to position 139, replacement with R or V;
at a position corresponding to position 141, replacement with M, Q, W or Y;
at a position corresponding to position 142, replacement with Q;
at a position corresponding to position 143, replacement with K;
at a position corresponding to position 144, replacement with G;
at a position corresponding to position 146, replacement with V;
at a position corresponding to position 147, replacement with G, I or M;
at a position corresponding to position 148, replacement with C, H or K;
at a position corresponding to position 150, replacement with L;
at a position corresponding to position 151, replacement with Q;
at a position corresponding to position 152, replacement with A, I, M or T;
at a position corresponding to position 154, replacement with R;
at a position corresponding to position 155, replacement with A, D, F, H, L, R, S or V;
at a position corresponding to position 156, replacement with A, C or Q;
at a position corresponding to position 158, replacement with H;
at a position corresponding to position 159, replacement with A, H, N, Q or S;
at a position corresponding to position 160, replacement with Y;
at a position corresponding to position 161, replacement with A or D;
at a position corresponding to position 162, replacement with L;
at a position corresponding to position 163, replacement with K, R or S;
at a position corresponding to position 165, replacement with F;
at a position corresponding to position 170, replacement with R;
at a position corresponding to position 174, replacement with W;
at a position corresponding to position 195, replacement with H, L or N;
at a position corresponding to position 196, replacement with T;
at a position corresponding to position 197, replacement with F;
at a position corresponding to position 198, replacement with L;
at a position corresponding to position 202, replacement with M;
at a position corresponding to position 204, replacement with P;
at a position corresponding to position 205, replacement with A, E, K, L, P, S or T;
at a position corresponding to position 206, replacement with I;
at a position corresponding to position 208, replacement with L, Q or R;
at a position corresponding to position 213, replacement with E or N;
at a position corresponding to position 215, replacement with A, D, E, H, T, V or W;
at a position corresponding to position 219, replacement with A, R, S or T;
at a position corresponding to position 220, replacement with V;
at a position corresponding to position 222, replacement with N;
at a position corresponding to position 234, replacement with M;
at a position corresponding to position 235, replacement with T;
at a position corresponding to position 237, replacement with Q;
at a position corresponding to position 240, replacement with Q;
at a position corresponding to position 247, replacement with I;
at a position corresponding to position 251, replacement with L or M;
at a position corresponding to position 255, replacement with R;
at a position corresponding to position 259, replacement with K or P;
at a position corresponding to position 260, replacement with G or M;
at a position corresponding to position 261, replacement with A or F;
at a position corresponding to position 263, replacement with T;
at a position corresponding to position 265, replacement with I;
at a position corresponding to position 271, replacement with V;
at a position corresponding to position 276, replacement with E;
at a position corresponding to position 277, replacement with A, C, E or H;
at a position corresponding to position 278, replacement with G, H, K or N;
at a position corresponding to position 282, replacement with G or Q;
at a position corresponding to position 284, replacement with A, Q or S;
at a position corresponding to position 285, replacement with M or Y;
at a position corresponding to position 290, replacement with M;
at a position corresponding to position 292, replacement with V;
at a position corresponding to position 305, replacement with D or N;
at a position corresponding to position 306, replacement with D;
at a position corresponding to position 309, replacement with E, H or L;
at a position corresponding to position 310, replacement with Q or R;
at a position corresponding to position 311, replacement with G or K;
at a position corresponding to position 315, replacement with T;
at a position corresponding to position 317, replacement with N;
at a position corresponding to position 318, replacement with K, M, N or Q;
at a position corresponding to position 320, replacement with R;
at a position corresponding to position 321, replacement with A, H or R;
at a position corresponding to position 328, replacement with L or R;
at a position corresponding to position 342, replacement with A;
at a position corresponding to position 343, replacement with V;
at a position corresponding to position 349, replacement with A or E;
at a position corresponding to position 359, replacement with E;
at a position corresponding to position 368, replacement with H or K;
at a position corresponding to position 369, replacement with H;
at a position corresponding to position 371, replacement with E, F, M or T;
at a position corresponding to position 373, replacement with S;
at a position corresponding to position 374, replacement with A or V;
at a position corresponding to position 375, replacement with T;
at a position corresponding to position 376, replacement with Y;
at a position corresponding to position 377, replacement with T;
at a position corresponding to position 379, replacement with H, S or T;
at a position corresponding to position 380, replacement with I, L, P, T or V;
at a position corresponding to position 388, replacement with H;
at a position corresponding to position 389, replacement with K;
at a position corresponding to position 393, replacement with L;
at a position corresponding to position 399, replacement with R or W;
at a position corresponding to position 401, replacement with G;
at a position corresponding to position 403, replacement with F;
at a position corresponding to position 406, replacement with N;
at a position corresponding to position 407, replacement with F, H, M, P or Q;
at a position corresponding to position 410, replacement with S;
at a position corresponding to position 412, replacement with G, P or S;
at a position corresponding to position 413, replacement with Q or T;
at a position corresponding to position 415, replacement with W;
at a position corresponding to position 417, replacement with L;
at a position corresponding to position 419, replacement with L;
at a position corresponding to position 421, replacement with I or M;
at a position corresponding to position 428, replacement with P;
at a position corresponding to position 431, replacement with A or G;
at a position corresponding to position 433, replacement with L or T;
at a position corresponding to position 434, replacement with I or M;
at a position corresponding to position 435, replacement with H;
at a position corresponding to position 438, replacement with A;
at a position corresponding to position 439, replacement with C or T;
at a position corresponding to position 440, replacement with M;
at a position corresponding to position 441, replacement with T or V;
at a position corresponding to position 442, replacement with P;
at a position corresponding to position 443, replacement with M;
at a position corresponding to position 445, replacement with Y;
at a position corresponding to position 446, replacement with C, D, E or G; and
at a position corresponding to position 447, replacement with D, E or G, each with reference to amino acid positions of the sequence set forth in SEQ ID NO:3.

4. The modified PH20 polypeptide of claim 1, wherein the modified PH20 polypeptide retains at least 60%, 65%, 70%, 75%, 80%, 85%, 90%, 95% or more of its hyaluronidase activity after incubation at 52° C. for 10 minutes compared to its hyaluronidase activity after incubation at 4° C. for 10 minutes.

5. The modified PH20 polypeptide of claim 1, comprising at least one amino acid replacement selected from among replacement with: glycine (G) at a position corresponding to position 11; A at a position corresponding to position 15; V at a position corresponding to position 15; R at a position corresponding to position 26; S at a position corresponding to position 26; E at a position corresponding to position 27; H at a position corresponding to position 27; H at a position corresponding to position 29; S at a position corresponding to position 29; A at a position corresponding to position 30; P at a position corresponding to position 30; G at a position corresponding to position 31; L at a position corresponding to position 31; Q at a position corresponding to position 32; W at a position corresponding to position 32; G at a position corresponding to position 33; M at a position corresponding to position 33; R at a position corresponding to position 33; W at a position corresponding to position 33; E at a position corresponding to position 34; H at a position corresponding to position 34; W at a position corresponding to position 34; K at a position corresponding to position 37; Y at a position corresponding to position 38; Q at a position corresponding to position 39; R at a position corresponding to position 39; T at a position corresponding to position 39; D at a position corresponding to position 41; T at a position corresponding to position 41; W at a position corresponding to position 41; G at a position corresponding to position 48; C at a position corresponding to position 50; D at a position corresponding to position 50; K at a position corresponding to position 58; R at a position corresponding to position 58; K at a position corresponding to position 60; F at a position corresponding to position 67; A at a position corresponding to position 69; Y at a position corresponding to position 69; Q at a position corresponding to position 83; D at a position corresponding to position 84; D at a position corresponding to position 86; E at a position corresponding to position 86; R at a position corresponding to position 86; P at a position corresponding to position 87; W at a position corresponding to position 90; V at a position corresponding to position 92; E at a position corresponding to position 93; S at a position corresponding to position 93; N at a position corresponding to position 94; F at a position corresponding to position 97; M at a position corresponding to position 98; S at a position corresponding to position 99; H at a position corresponding to position 102; G at a position corresponding to position 114; M at a position corresponding to position 118; S at a position corresponding to position 120; C at a position corresponding to position 131; L at a position corresponding to position 131; A at a position corresponding to position 132; W at a position corresponding to position 138; R at a position corresponding to position 139; V at a position corresponding to position 139; M at a position corresponding to position 141; Y at a position corresponding to position 141; K at a position corresponding to position 143; G at a position corresponding to position 144; V at a position corresponding to position 146; I at a position corresponding to position 147; M at a position corresponding to position 147; C at a position corresponding to position 148; H at a position corresponding to position 148; K at a position corresponding to position 148; L at a position corresponding to position 150; Q at a position corresponding to position 151; I at a position corresponding to position 152; M at a position corresponding to position 152; T at a position corresponding to position 152; R at a position corresponding to position 154; A at a position corresponding to position 155; D at a position corresponding to position 155; F at a position corresponding to position 155; H at a position corresponding to position 155; L at a position corresponding to position 155; R at a position corresponding to position 155; S at a position corresponding to position 155; H at a position corresponding to position 158; A at a position corresponding to position 159; H at a position corresponding to position 159; N at a position corresponding to position 159; Q at a position corresponding to position 159; S at a position corresponding to position 159; Y at a position corresponding to position 160; A at a position corresponding to position 161; L at a position corresponding to position 162; K at a position corresponding to position 163; R at a position corresponding to position 163; S at a position corresponding to position 163; F at a position corresponding to position 165; W at a position corresponding to position 174; H at a position corresponding to position 195; L at a position corresponding to position 195; T at a position corresponding to position 196; F at a position corresponding to position 197; L at a position corresponding to position 198; P at a position corresponding to position 204; A at a position corresponding to position 205; E at a position corresponding to position 205; K at a position corresponding to position 205; L at a position corresponding to position 205; T at a position corresponding to position 205; I at a position corresponding to position 206; Q at a position corresponding to position 208; R at a position corresponding to position 208; E at a position corresponding to position 213; N at a position corresponding to position 213; E at a position corresponding to position 215; H at a position corresponding to position 215; T at a position corresponding to position 215; N at a position corresponding to position 222; T at a position corresponding to position 235; Q at a position corresponding to position 237; Q at a position corresponding to position 240; I at a position corresponding to position 247; L at a position corresponding to position 251; Mat a position corresponding to position 251; K at a position corresponding to position 259; P at a position corresponding to position 259; M at a position corresponding to position 260; A at a position corresponding to position 261; F at a position corresponding to position 261; T at a position corresponding to position 263; V at a position corresponding to position 271; Eat a position corresponding to position 276; A at a position corresponding to position 277; C at a position corresponding to position 277; N at a position corresponding to position 278; Q at a position corresponding to position 282; A at a position corresponding to position 284; Q at a position corresponding to position 284; S at a position corresponding to position 284; M at a position corresponding to position 285; V at a position corresponding to position 292; N at a position corresponding to position 305; D at a position corresponding to position 306; R at a position corresponding to position 310; G at a position corresponding to position 311; T at a position corresponding to position 315; N at a position corresponding to position 317; A at a position corresponding to position 321; R at a position corresponding to position 321; L at a position corresponding to position 328; R at a position corresponding to position 328; A at a position corresponding to position 342; H at a position corresponding to position 368; K at a position corresponding to position 368; H at a position corresponding to position 369; F at a position corresponding to position 371; S at a position corresponding to position 373; T at a position corresponding to position 377; H at a position corresponding to position 379; S at a position corresponding to position 379; T at a position corresponding to position 379; I at a position corresponding to position 380; L at a position corresponding to position 380; P at a position corresponding to position 380; T at a position corresponding to position 380; H at a position corresponding to position 388; N at a position corresponding to position 406; F at a position corresponding to position 407; Q at a position corresponding to position 407; S at a position corresponding to position 410; G at a position corresponding to position 412; P at a position corresponding to position 412; S at a position corresponding to position 412; Q at a position corresponding to position 413; M at a position corresponding to position 421; P at a position corresponding to position 428; A at a position corresponding to position 431; L at a position corresponding to position 433; T at a position corresponding to position 433; A at a position corresponding to position 438; C at a position corresponding to position 439; T at a position corresponding to position 441; M at a position corresponding to position 443; Y at a position corresponding to position 445; C at a position corresponding to position 446; D at a position corresponding to position 446; E at a position corresponding to position 446; G at a position corresponding to position 446; E at a position corresponding to position 447; and G at a position corresponding to position 447, with reference to amino acid residue positions of the sequence set forth in SEQ ID NO:3.

6. The modified PH20 polypeptide of claim 1 that contains 1, 2, 3, 4, 5, 6, 7, 8, 9, 10, 11, 12, 13, 14, 15, 16, 17, 18, 19 or 20 amino acid replacements compared to the unmodified PH20 polypeptide.

7. The modified PH20 polypeptide of claim 6, comprising at least 2 amino acid replacements selected from among replacement with: glycine (G) at a position corresponding to position 11; A at a position corresponding to position 15; V at a position corresponding to position 15; R at a position corresponding to position 26; S at a position corresponding to position 26; E at a position corresponding to position 27; H at a position corresponding to position 27; H at a position corresponding to position 29; S at a position corresponding to position 29; A at a position corresponding to position 30; P at a position corresponding to position 30; G at a position corresponding to position 31; L at a position corresponding to position 31; Q at a position corresponding to position 32; W at a position corresponding to position 32; G at a position corresponding to position 33; M at a position corresponding to position 33; R at a position corresponding to position 33; W at a position corresponding to position 33; E at a position corresponding to position 34; H at a position corresponding to position 34; W at a position corresponding to position 34; K at a position corresponding to position 37; Y at a position corresponding to position 38; Q at a position corresponding to position 39; R at a position corresponding to position 39; T at a position corresponding to position 39; D at a position corresponding to position 41; T at a position corresponding to position 41; W at a position corresponding to position 41; G at a position corresponding to position 48; C at a position corresponding to position 50; D at a position corresponding to position 50; K at a position corresponding to position 58; R at a position corresponding to position 58; K at a position corresponding to position 60; F at a position corresponding to position 67; A at a position corresponding to position 69; Y at a position corresponding to position 69; Q at a position corresponding to position 83; D at a position corresponding to position 84; D at a position corresponding to position 86; E at a position corresponding to position 86; R at a position corresponding to position 86; P at a position corresponding to position 87; W at a position corresponding to position 90; V at a position corresponding to position 92; E at a position corresponding to position 93; S at a position corresponding to position 93; N at a position corresponding to position 94; F at a position corresponding to position 97; M at a position corresponding to position 98; S at a position corresponding to position 99; H at a position corresponding to position 102; G at a position corresponding to position 114; M at a position corresponding to position 118; S at a position corresponding to position 120; C at a position corresponding to position 131; L at a position corresponding to position 131; A at a position corresponding to position 132; W at a position corresponding to position 138; R at a position corresponding to position 139; V at a position corresponding to position 139; M at a position corresponding to position 141; Y at a position corresponding to position 141; K at a position corresponding to position 143; G at a position corresponding to position 144; V at a position corresponding to position 146; I at a position corresponding to position 147; M at a position corresponding to position 147; C at a position corresponding to position 148; H at a position corresponding to position 148; K at a position corresponding to position 148; L at a position corresponding to position 150; Q at a position corresponding to position 151; I at a position corresponding to position 152; M at a position corresponding to position 152; T at a position corresponding to position 152; R at a position corresponding to position 154; A at a position corresponding to position 155; D at a position corresponding to position 155; F at a position corresponding to position 155; H at a position corresponding to position 155; L at a position corresponding to position 155; R at a position corresponding to position 155; S at a position corresponding to position 155; H at a position corresponding to position 158; A at a position corresponding to position 159; H at a position corresponding to position 159; N at a position corresponding to position 159; Q at a position corresponding to position 159; S at a position corresponding to position 159; Y at a position corresponding to position 160; A at a position corresponding to position 161; L at a position corresponding to position 162; K at a position corresponding to position 163; R at a position corresponding to position 163; S at a position corresponding to position 163; F at a position corresponding to position 165; W at a position corresponding to position 174; H at a position corresponding to position 195; L at a position corresponding to position 195; T at a position corresponding to position 196; F at a position corresponding to position 197; L at a position corresponding to position 198; P at a position corresponding to position 204; A at a position corresponding to position 205; E at a position corresponding to position 205; K at a position corresponding to position 205; L at a position corresponding to position 205; T at a position corresponding to position 205; I at a position corresponding to position 206; Q at a position corresponding to position 208; R at a position corresponding to position 208; E at a position corresponding to position 213; N at a position corresponding to position 213; E at a position corresponding to position 215; H at a position corresponding to position 215; T at a position corresponding to position 215; N at a position corresponding to position 222; T at a position corresponding to position 235; Q at a position corresponding to position 237; Q at a position corresponding to position 240; I at a position corresponding to position 247; L at a position corresponding to position 251; M at a position corresponding to position 251; K at a position corresponding to position 259; P at a position corresponding to position 259; M at a position corresponding to position 260; A at a position corresponding to position 261; F at a position corresponding to position 261; T at a position corresponding to position 263; V at a position corresponding to position 271; E at a position corresponding to position 276; A at a position corresponding to position 277; C at a position corresponding to position 277; N at a position corresponding to position 278; Q at a position corresponding to position 282; A at a position corresponding to position 284; Q at a position corresponding to position 284; S at a position corresponding to position 284; M at a position corresponding to position 285; V at a position corresponding to position 292; N at a position corresponding to position 305; D at a position corresponding to position 306; R at a position corresponding to position 310; G at a position corresponding to position 311; Tat a position corresponding to position 315; N at a position corresponding to position 317; A at a position corresponding to position 321; R at a position corresponding to position 321; L at a position corresponding to position 328; R at a position corresponding to position 328; A at a position corresponding to position 342; H at a position corresponding to position 368; K at a position corresponding to position 368; H at a position corresponding to position 369; F at a position corresponding to position 371; S at a position corresponding to position 373; T at a position corresponding to position 377; H at a position corresponding to position 379; S at a position corresponding to position 379; T at a position corresponding to position 379; I at a position corresponding to position 380; L at a position corresponding to position 380; P at a position corresponding to position 380; T at a position corresponding to position 380; H at a position corresponding to position 388; N at a position corresponding to position 406; F at a position corresponding to position 407; Q at a position corresponding to position 407; S at a position corresponding to position 410; G at a position corresponding to position 412; P at a position corresponding to position 412; S at a position corresponding to position 412; Q at a position corresponding to position 413; M at a position corresponding to position 421; P at a position corresponding to position 428; A at a position corresponding to position 431; L at a position corresponding to position 433; T at a position corresponding to position 433; A at a position corresponding to position 438; C at a position corresponding to position 439; T at a position corresponding to position 441; M at a position corresponding to position 443; Y at a position corresponding to position 445; C at a position corresponding to position 446; D at a position corresponding to position 446; E at a position corresponding to position 446; G at a position corresponding to position 446; E at a position corresponding to position 447; and G at a position corresponding to position 447, with reference to amino acid residue positions of the sequence set forth in SEQ ID NO:3.

8. The modified PH20 polypeptide of claim 1, wherein the amino acid replacement(s) is/are selected from among replacement with: alanine (A) at a position corresponding to position 15; V at a position corresponding to position 15; R at a position corresponding to position 26; E at a position corresponding to position 27; S at a position corresponding to position 29; G at a position corresponding to position 31; L at a position corresponding to position 31; Q at a position corresponding to position 32; G at a position corresponding to position 33; M at a position corresponding to position 33; R at a position corresponding to position 33; W at a position corresponding to position 33; E at a position corresponding to position 34; H at a position corresponding to position 34; Y at a position corresponding to position 38; R at a position corresponding to position 39; W at a position corresponding to position 41; G at a position corresponding to position 48; C at a position corresponding to position 50; R at a position corresponding to position 58; A at a position corresponding to position 69; D at a position corresponding to position 86; E at a position corresponding to position 86; R at a position corresponding to position 86; W at a position corresponding to position 90; E at a position corresponding to position 93; S at a position corresponding to position 93; F at a position corresponding to position 97; S at a position corresponding to position 99; S at a position corresponding to position 120; L at a position corresponding to position 131; A at a position corresponding to position 132; W at a position corresponding to position 138; R at a position corresponding to position 139; V at a position corresponding to position 139; M at a position corresponding to position 141; Y at a position corresponding to position 141; K at a position corresponding to position 143; V at a position corresponding to position 146; I at a position corresponding to position 147; M at a position corresponding to position 147; C at a position corresponding to position 148; H at a position corresponding to position 148; K at a position corresponding to position 148; L at a position corresponding to position 150; Q at a position corresponding to position 151; I at a position corresponding to position 152; M at a position corresponding to position 152; T at a position corresponding to position 152; R at a position corresponding to position 154; A at a position corresponding to position 155; F at a position corresponding to position 155; L at a position corresponding to position 155; R at a position corresponding to position 155; H at a position corresponding to position 158; H at a position corresponding to position 159; N at a position corresponding to position 159; Q at a position corresponding to position 159; S at a position corresponding to position 159; Y at a position corresponding to position 160; R at a position corresponding to position 163; F at a position corresponding to position 165; W at a position corresponding to position 174; L at a position corresponding to position 198; P at a position corresponding to position 204; A at a position corresponding to position 205; L at a position corresponding to position 205; T at a position corresponding to position 205; I at a position corresponding to position 206; Q at a position corresponding to position 208; R at a position corresponding to position 208; N at a position corresponding to position 213; E at a position corresponding to position 215; T at a position corresponding to position 215; T at a position corresponding to position 235; Q at a position corresponding to position 237; Q at a position corresponding to position 240; L at a position corresponding to position 251; K at a position corresponding to position 259; M at a position corresponding to position 260; A at a position corresponding to position 261; F at a position corresponding to position 261; T at a position corresponding to position 263; V at a position corresponding to position 271; E at a position corresponding to position 276; A at a position corresponding to position 277; C at a position corresponding to position 277; A at a position corresponding to position 284; Q at a position corresponding to position 284; S at a position corresponding to position 284; V at a position corresponding to position 292; N at a position corresponding to position 305; D at a position corresponding to position 306; R at a position corresponding to position 310; T at a position corresponding to position 315; R at a position corresponding to position 328; A at a position corresponding to position 342; K at a position corresponding to position 368; H at a position corresponding to position 369; S at a position corresponding to position 373; H at a position corresponding to position 379; S at a position corresponding to position 379; T at a position corresponding to position 379; I at a position corresponding to position 380; L at a position corresponding to position 380; P at a position corresponding to position 380; T at a position corresponding to position 380; H at a position corresponding to position 388; G at a position corresponding to position 412; P at a position corresponding to position 412; S at a position corresponding to position 412; Q at a position corresponding to position 413; T at a position corresponding to position 433; A at a position corresponding to position 438; T at a position corresponding to position 441; M at a position corresponding to position 443; Y at a position corresponding to position 445; C at a position corresponding to position 446; E at a position corresponding to position 447; and G at a position corresponding to position 447, with reference to amino acid residue positions of the sequence set forth in SEQ ID NO: 3.

9. The modified PH20 polypeptide of claim 1, wherein the amino acid replacement(s) is/are selected from among replacement with: alanine (A) at a position corresponding to position 15; V at a position corresponding to position 15; R at a position corresponding to position 26; E at a position corresponding to position 27; S at a position corresponding to position 29; G at a position corresponding to position 31; G at a position corresponding to position 33; M at a position corresponding to position 33; R at a position corresponding to position 33; W at a position corresponding to position 33; E at a position corresponding to position 34; H at a position corresponding to position 34; Y at a position corresponding to position 38; R at a position corresponding to position 39; G at a position corresponding to position 48; R at a position corresponding to position 86; W at a position corresponding to position 90; E at a position corresponding to position 93; S at a position corresponding to position 93; F at a position corresponding to position 97; S at a position corresponding to position 120; L at a position corresponding to position 131; A at a position corresponding to position 132; R at a position corresponding to position 139; M at a position corresponding to position 141; Y at a position corresponding to position 141; K at a position corresponding to position 143; I at a position corresponding to position 147; M at a position corresponding to position 147; C at a position corresponding to position 148; H at a position corresponding to position 148; K at a position corresponding to position 148; M at a position corresponding to position 152; T at a position corresponding to position 152; R at a position corresponding to position 154; A at a position corresponding to position 155; F at a position corresponding to position 155; L at a position corresponding to position 155; N at a position corresponding to position 159; S at a position corresponding to position 159; Y at a position corresponding to position 160; R at a position corresponding to position 163; F at a position corresponding to position 165; W at a position corresponding to position 174; L at a position corresponding to position 198; P at a position corresponding to position 204; A at a position corresponding to position 205; L at a position corresponding to position 205; T at a position corresponding to position 205; I at a position corresponding to position 206; R at a position corresponding to position 208; N at a position corresponding to position 213; E at a position corresponding to position 215; T at a position corresponding to position 215; Q at a position corresponding to position 240; L at a position corresponding to position 251; K at a position corresponding to position 259; M at a position corresponding to position 260; A at a position corresponding to position 261; F at a position corresponding to position 261; T at a position corresponding to position 263; V at a position corresponding to position 271; A at a position corresponding to position 277; C at a position corresponding to position 277; A at a position corresponding to position 284; Q at a position corresponding to position 284; S at a position corresponding to position 284; V at a position corresponding to position 292; T at a position corresponding to position 315; A at a position corresponding to position 342; H at a position corresponding to position 369; H at a position corresponding to position 379; S at a position corresponding to position 379; T at a position corresponding to position 379; L at a position corresponding to position 380; P at a position corresponding to position 380; T at a position corresponding to position 380; H at a position corresponding to position 388; G at a position corresponding to position 412; P at a position corresponding to position 412; S at a position corresponding to position 412; T at a position corresponding to position 433; A at a position corresponding to position 438; T at a position corresponding to position 441; M at a position corresponding to position 443; Y at a position corresponding to position 445; C at a position corresponding to position 446; E at a position corresponding to position 447; and G at a position corresponding to position 447, with reference to amino acid residue positions of the sequence set forth in SEQ ID NO: 3.

10. The modified PH20 polypeptide of claim 1, wherein the amino acid replacement(s) is/are selected from among replacement with: glutamic acid (E) at a position corresponding to position 27; A at a position corresponding to position 132; K at a position corresponding to position 143; M at a position corresponding to position 147; C at a position corresponding to position 148; H at a position corresponding to position 148; Y at a position corresponding to position 160; P at a position corresponding to position 204; A at a position corresponding to position 205; I at a position corresponding to position 206; T at a position corresponding to position 215; M at a position corresponding to position 260; A at a position corresponding to position 261; F at a position corresponding to position 261; T at a position corresponding to position 263; A at a position corresponding to position 284; T at a position corresponding to position 315; and S at a position corresponding to position 379, with reference to amino acid residue positions of the sequence set forth in SEQ ID NO: 3.

11. The modified PH20 polypeptide of claim 1, wherein the amino acid replacement(s) is/are selected from among replacement with: P at a position corresponding to position 30; R at a position corresponding to position 58; K at a position corresponding to position 60; K at a position corresponding to position 143; I at a position corresponding to position 147; P at a position corresponding to position 204; T at a position corresponding to position 215; T at a position corresponding to position 235; A at a position corresponding to position 261; G at a position corresponding to position 311; T at a position corresponding to position 315; and H at a position corresponding to position 369, with reference to amino acid residue positions of the sequence set forth in SEQ ID NO: 3.

12. The modified PH20 polypeptide of claim 11, wherein the amino acid replacement(s) is/are selected from among replacement with: P at a position corresponding to position 30; K at a position corresponding to position 60; I at a position corresponding to position 147; T at a position corresponding to position 215; T at a position corresponding to position 235; G at a position corresponding to position 311; T at a position corresponding to position 315; and H at a position corresponding to position 369, with reference to amino acid residue positions of the sequence set forth in SEQ ID NO: 3.

13. The modified PH20 polypeptide of claim 1, wherein the amino acid replacement(s) is/are in an unmodified PH20 polypeptide that has the sequence of amino acids set forth in any of SEQ ID NOS: 3, 7, 10, 12, 14, 32-66, 69, 72, 388, 390, 392, or 400.

14. The modified PH20 polypeptide of claim 1, wherein the amino acid replacement(s) is/are in an unmodified PH20 polypeptide that has the sequence of amino acids set forth in SEQ ID NOS: 3, 7, 32-66, 69 or 72, or a sequence of amino acids that exhibits at least 91% sequence identity to any of SEQ ID NOS: 3, 7, 32-66, 69 or 72.

15. The modified PH20 polypeptide of claim 1, wherein the modified PH20 polypeptide exhibits at least 95%-amino acid sequence identity to the sequence of amino acids set forth in SEQ ID NO:3.

16. The modified PH20 polypeptide of claim 1 that is a mature PH20 polypeptide lacking the signal sequence.

17. The modified PH20 polypeptide of claim 1, consisting of the sequence of amino acids set forth in any of SEQ ID NOS: 73-386 or a sequence of amino acids that exhibits at least 95% or more sequence identity to a sequence of amino acids set forth in any of SEQ ID NOS: 73-386 and containing the amino acid replacement(s).

18. The modified PH20 polypeptide of claim 1 that is substantially purified or isolated.

19. The modified PH20 polypeptide of claim 1 that comprises one or more of glycosylation, sialation, albumination, farnysylation, carboxylation, hydroxylation and/or phosphorylation.

20. The modified PH20 polypeptide of claim 19, wherein the modified PH20 polypeptide is glycosylated.

21. The modified PH20 polypeptide of claim 1 that is conjugated to a polymer.

22. The modified PH20 polypeptide of claim 21, wherein the polymer is dextran or PEG.

23. The modified PH20 polypeptide of claim 1, wherein the modified PH20 polypeptide is conjugated to a moiety selected from among a multimerization domain, a toxin, a detectable label and a drug.

24. The modified PH20 polypeptide of claim 23, wherein the modified PH20 polypeptide is conjugated to an Fc domain.

25. A conjugate comprising the modified PH20 polypeptide of claim 1 linked directly or indirectly via a linker to a targeting agent.

26. A nucleic acid molecule, encoding a modified PH20 polypeptide of claim 1.

27. A vector, comprising the nucleic acid molecule of claim 26.

28. A cell, comprising the vector of claim 27.

29. A method of producing a modified PH20 polypeptide, comprising:
introducing the nucleic acid of claim 26 into a cell capable of incorporating N-linked sugar moieties into the polypeptide; and
culturing the cell under conditions whereby an encoded modified PH20 polypeptide is produced and secreted by the cell; and
optionally recovering the expressed PH20 polypeptide.

30. A pharmaceutical composition, comprising a modified PH20 polypeptide of claim 1 in a pharmaceutically acceptable excipient.

31. The pharmaceutical composition of claim 30, comprising a further therapeutically active agent.

32. The pharmaceutical composition of claim 31, wherein the therapeutically active agent is selected from among a protein, a nucleic acid, a drug, a small molecule or an organic molecule.

33. A combination comprising:
a first composition comprising a pharmaceutical composition of claim 30; and
a second composition, comprising a therapeutically active agent.

34. The combination of claim 33, wherein the therapeutic agent is a protein, a nucleic acid, a drug, a small molecule or an organic molecule.

35. A system for the non-refrigerated storage of a stable PH20 hyaluronidase formulation, comprising:
a) the modified PH20 polypeptide of claim 1; and
b) a container suitable for storage without refrigeration.

36. The system of claim 35, wherein the container is selected from among a vial, syringe, tube or bag.

37. A method of preparing a pharmaceutical composition comprising a PH20 hyaluronidase that can be stored for direct administration without refrigeration, comprising
a) providing a PH20 polypeptide of claim 1; and
b) formulating the polypeptide as a liquid with a pharmaceutically acceptable buffering agent for parenteral administration.

38. The method of claim 37, wherein parenteral administration is intravenous or subcutaneous administration.

39. A method for treating a hyaluronan-associated disease or condition or for increasing delivery of a therapeutic agent to a subject, comprising administering to a subject a pharmaceutical composition of claim 30.

40. The method of claim 39, wherein the method is for treating a hyaluronan-associated disease or condition and the hyaluronan-associated disease or condition is an inflammatory disease or a tumor or cancer.

41. The method of claim 40, wherein the tumor is a solid tumor.

42. The method of claim 39, wherein the administration is subcutaneous.

43. The method of claim 39, wherein the administration is intravenous.

44. A method for identifying or selecting a modified hyaluronan-degrading enzyme that exhibits thermal stability, comprising:
a) testing the activity of a modified hyaluronan-degrading enzyme or a member of a collection of modified hyaluronan-degrading enzymes after incubation at a temperature for a predetermined time that provides a thermal stress condition to the unmodified hyaluronan-degrading enzyme not containing a modification;
b) testing the activity of the modified hyaluronan-degrading enzyme or a member of a collection of modified hyaluronan-degrading enzymes after incubation at 2° C. to 8° C., wherein in the activity is tested under the same conditions as a) except for the difference in temperature; and
c) selecting or identifying a modified hyaluronan-degrading enzyme that exhibits activity in a) that is at least 50% of the activity in b).

45. The method of claim 44, further comprising:
d) comparing the activity in b) of the selected or identified modified hyaluronan-degrading enzyme to the activity of the unmodified hyaluronan-degrading enzyme tested under the same conditions; and
e) identifying or selecting a modified hyaluronan-degrading enzyme that exhibits at least 40% of the activity compared to the unmodified hyaluronan-degrading enzyme.

46. The method of claim 44, wherein the activity is hyaluronidase activity.

47. The method of claim 44, wherein:
the thermal stress condition is a temperature that is or is greater than the T₅₀ of the unmodified hyaluronan-degrading enzyme not containing a modification as determined in a thermal challenge assay for the predetermined time; or
the thermal stress condition is a temperature that is or is greater than the melting temperature (Tm) of the unmodified hyaluronan-degrading enzyme not containing a modification or modifications.

48. The method of claim 44, wherein activity in a) is tested at a temperature that is greater than 44° C.

49. The method of claim 44, wherein the unmodified hyaluronan degrading enzyme is a hyaluronidase and the hyaluronidase is a PH20 hyaluronidase or truncated form thereof lacking a C-terminal glycosylphosphatidylinositol (GPI) anchor attachment site or a portion of the GPI anchor attachment site, whereby the truncated form exhibits hyaluronidase activity.

50. The method of claim 49, wherein the unmodified hyaluronidase is a PH20 selected from a human, monkey, bovine, ovine, rat, fox, mouse or guinea pig PH20.

51. The method of claim 49, wherein the unmodified hyaluronidase is a human PH20 or a C-terminal truncated form thereof.
